SEQUENCE LISTING (1316 sequences)

SEQ ID NO: 1 - DNA, 700 bp, Homo sapiens
  source 1..700: mol_type = other DNA
ctacatggacgtctggggcaaagggaccacggtcaccgtctcctcaggtaagaatggccactctagggcctttgttttctgctactgcctgtggggtttcctgagcattgcaggttggtcctcggggcatgttccgaggggacctgggcggactggccaggaggggatgggcactggggtgccttgaggatctgggagcctctgtggattttccgatgcctttggaaaatgggactcaggttgggtgcgtctgatggagtaactgagcctgggggcttggggagccacatttggacgagatgcctgaacaaaccaggggtcttagtgatggctgaggaatgtgtctcaggagcggtgtctgtaggactgcaagatcgctgcacagcagcgaatcgtgaaatattttctttagaattatgaggtgcgctgtgtgtcaacctgcatcttaaattctttattggctggaaagagaactgtcggagtgggtgaatccagccaggagggacgcgtagccccggtcttgatgagagcagggttgggggcaggggtagcccagaaacggtggctgccgtcctgacaggggcttagggaggctccaggacctcagtgccttgaagctggtttccatgagaaaaggattgtttatcttaggaggcatgcttactgttaaaagacaggatatgtttgaagtggcttctga

SEQ ID NO: 2 - DNA, 1308 bp, Homo sapiens
  source 1..1308: mol_type = other DNA
ctcactttaggataagttttaggtaaaatgtgcatcattatcctgaattatttcagttaagcatgttagttggtggcataagagaaaactcaatcagatagtgctgaagacaggactgtggagacaccttagaaggacagattctgttccgaatcaccgatgcggcgtcagcaggactggcctagcggaggctctgggagggtggctgccaggcccggcctgggctttgggtctccccggactacccagagctgggatgcgtggcttctgctgccgggccgactggctgctcaggccccagcccttgttaatggacttggaggaatgattccatgccaaagctttgcaaggctcgcagtgaccaggcgcccgacatggtaagagacaggcagccgccgctgctgcatttgcttctcttaaaactttgtatttgacgtcttatttccactagaaggggaactggtcttaattgcttgatgaagagcaggagactcatttatgtgagtcttttgagtgaccattgtctgggtcactcccatttaactttccctaaagcccatttgaaggagaggtcgcacgagctgctccacaacctctgaatggggatggcatgggtaatgatgcttgagaacataccaagccccactggcatcgcccttgtctaagtcattgactgtaggtcatcatcgcacccttgaaagtagcccatgccttccaaagcgatttatggtaaatggcagaattttaagtggcaaattcagataaaatgcatttcttggttgtttccaatgatgactgttatctagagggaatttaaaggcaggggtttactgcagactcagaagggaggggatgctccgggaaggtggaggctctgagcatctcaataccctcctcttggtgcagaagatatgctgccacttctagagcaaggggacctgctcatttttatcacagcacaggctcctaaattcttggtctcattctcaagatgttttaatgactttaaagcagcaaagaaatattccacccaggtagtggagggtggtaatgattggtaatgctttggaaccaaaacccaggtggcgctggggcaggactgcagggaactggggtatcaagtagagggagacaaaagatggaagccagcctggctgtgcaggaacccggcaatgagatggctttagctgagacaagcaggtctggtgggctgaccatttctggccatgacaactccatccagctttcagaaatggactcagatgggcaaaactgacctaagctgacctagactaaacaaggctgaac

SEQ ID NO: 3 - protein, 115 aa, Homo sapiens
GGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 114 aa, Homo sapiens
ELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - DNA, 345 bp, Homo sapiens
  source 1..345: mol_type = other DNA
ggcggagggcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 7 - DNA, 336 bp, Homo sapiens
  source 1..336: mol_type = other DNA
cgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 8 - protein, 42 aa, Homo sapiens
KRGRKKLLYIFKQPFMRPVQTTQEEDGCSCRFPEEEEGGCEL

SEQ ID NO: 9 - protein, 43 aa, Homo sapiens
VKRGRKKLLYIFKQPFMRPVQTTQEEDGCSCRFPEEEEGGCEL

SEQ ID NO: 10 - protein, 41 aa, Homo sapiens
VKRGRKKLLYIFKQPFMRPVQTTQEEDGCSCRFPEEEEGGC

SEQ ID NO: 11 - DNA, 126 bp, Homo sapiens
  source 1..126: mol_type = other DNA
aagagaggacggaagaagctgctgtatatctttaagcagccttttatgagacctgtccaaacaacccaagaggaggacgggtgttcctgtcgcttcccagaggaggaggaaggagggtgcgagctg

SEQ ID NO: 12 - DNA, 126 bp, Homo sapiens
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 13 - DNA, 129 bp, Homo sapiens
  source 1..129: mol_type = other DNA
gtgaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 14 - protein, 28 aa, Homo sapiens
MFWVLVVVGGVLACYSLLVTVAFIIFWV

SEQ ID NO: 15 - protein, 27 aa, Homo sapiens
MFWVLVVVGGVLACYSLLVTVAFIIFW

SEQ ID NO: 16 - protein, 26 aa, Homo sapiens
FWVLVVVGGVLACYSLLVTVAFIIFW

SEQ ID NO: 17 - DNA, 84 bp, Homo sapiens
  source 1..84: mol_type = other DNA
atgttctgggtgctggtggtggtgggcggggtgctggcctgctacagcctgctggtgacagtggccttcatcatcttttgggta

SEQ ID NO: 18 - DNA, 84 bp, Homo sapiens
  source 1..84: mol_type = other DNA
atgttctgggtgctggtggtggtcggaggcgtgctggcctgctacagcctgctggtcaccgtggccttcatcatcttttgggtg

SEQ ID NO: 19 - DNA, 81 bp, Homo sapiens
  source 1..81: mol_type = other DNA
atgttctgggtgctggtggtggtcggaggcgtgctggcctgctacagcctgctggtcaccgtggccttcatcatcttttgg

SEQ ID NO: 20 - DNA, 81 bp, Homo sapiens
  source 1..81: mol_type = other DNA
atgttctgggtgctggtggtggtgggcggggtgctggcctgctacagcctgctggtgacagtggccttcatcatcttttgg

SEQ ID NO: 21 - protein, 12 aa, Homo sapiens
ESKYGPPCPPCP

SEQ ID NO: 22 - DNA, 36 bp, Homo sapiens
  source 1..36: mol_type = other DNA
gaatctaagtacggaccgccctgccccccttgccct

SEQ ID NO: 23 - DNA, 36 bp, Homo sapiens
  source 1..36: mol_type = other DNA
gagtctaagtacggaccgccttgcccaccgtgccca

SEQ ID NO: 24 - DNA, 36 bp, Homo sapiens
  source 1..36: mol_type = other DNA
gagtctaagtacggaccgccctgccccccttgccct

SEQ ID NO: 25 - protein, 103 aa, Homo sapiens
GPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKA

SEQ ID NO: 26 - protein, 108 aa, Homo sapiens
APPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKA

SEQ ID NO: 27 - DNA, 311 bp, Homo sapiens
  source 1..311: mol_type = other DNA
ggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttccagagcacctaccgggtggtgtccgtgctgaccgtgctgcaccaggactggctgaacggcaaggaatacaagtgtaaggtgtccaacaagggcctgcccagcagcatcgagaaaaccatcagcaaggccaa

SEQ ID NO: 28 - DNA, 326 bp, Homo sapiens
  source 1..326: mol_type = other DNA
gccccccccgtggccggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttccagagcacctaccgggtggtgtccgtgctgaccgtgctgcaccaggactggctgaacggcaaggaatacaagtgtaaggtgtccaacaagggcctgcccagcagcatcgagaaaaccatcagcaaggccaa

SEQ ID NO: 29 - protein, 104 aa, Homo sapiens
KGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSL

SEQ ID NO: 30 - protein, 108 aa, Homo sapiens
KGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - DNA, 311 bp, Homo sapiens
  source 1..311: mol_type = other DNA
agggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctg

SEQ ID NO: 32 - DNA, 323 bp, Homo sapiens
  source 1..323: mol_type = other DNA
agggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctgagcctgggcaag

SEQ ID NO: 33 - protein, 119 aa, Homo sapiens
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - DNA, 357 bp, Homo sapiens
  source 1..357: mol_type = other DNA
gaatctaagtacggaccgccctgccccccttgccctggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctgagcctgggcaag

SEQ ID NO: 35 - protein, 224 aa, Homo sapiens
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSL

SEQ ID NO: 36 - DNA, 672 bp, Homo sapiens
  source 1..672: mol_type = other DNA
gaatctaagtacggaccgccctgccccccttgccctgccccccccgtggccggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttccagagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaatcaggtgtccctgacctgcctggtgaagggcttctaccccagcgacatcgccgtggagtgggagagcaacggccagcccgagaacaactacaagaccaccccccctgtgctggacagcgacggcagcttcttcctgtacagcaggctgaccgtggacaagagccggtggcaggagggcaacgtctttagctgctccgtgatgcacgaggccctgcacaaccactacacccagaagagcctgtccctg

SEQ ID NO: 37 - protein, 24 aa, Thosea asigna virus
LEGGGEGRGSLLTCGDVEENPGPR

SEQ ID NO: 38 - protein, 21 aa, Thosea asigna virus
GSGEGRGSLLTCGDVEENPGP

SEQ ID NO: 39 - protein, 22 aa, Porcine Teschovirus-1
GSGATNFSLLKQAGDVEENPGP

SEQ ID NO: 40 - protein, 23 aa, Equine rhinitis A virus
GSGQCTNYALLKLAGDVESNPGP

SEQ ID NO: 41 - protein, 25 aa, Foot-and-mouth disease virus
GSGVKQTLNFDLLKLAGDVESNPGP

SEQ ID NO: 42 - DNA, 72 bp, Homo sapiens
  source 1..72: mol_type = other DNA
ctcgagggcggcggagagggcagaggaagtcttctaacatgcggtgacgtggaggagaatcccggccctagg

SEQ ID NO: 43 - DNA, 72 bp, Homo sapiens
  source 1..72: mol_type = other DNA
ctcgagggcggcggagagggcagaggaagtcttctaacatgcggtgacgtggaggagaatccaggccctagg

SEQ ID NO: 44 - protein, 186 aa, Merkel cell polyomavirus
MDLVLNRKEREALCKLLEIAPNCYGNIPLMKAAFKRSCLKHHPDKGGNPVIMMELNTLWSKFQQNIHKLRSDFSMFDEVSTKFPWEEYGTLKDYMQSGYNARFCRGPGCMLKQLRDSKCACISCKLSRQHCSLKTLKQKNCLTWGECFCYQCFILWFGFPPTWESFDWWQKTLEETDYCLLHLHLF

SEQ ID NO: 45 - protein, 817 aa, Merkel cell polyomavirus
MDLVLNRKEREALCKLLEIAPNCYGNIPLMKAAFKRSCLKHHPDKGGNPVIMMELNTLWSKFQQNIHKLRSDFSMFDEVDEAPIYGTTKFKEWWRSGGFSFGKAYEYGPNPHGTNSRSRKPSSNASRGAPSGSSPPHSQSSSSGYGSFSASQASDSQSRGPDIPPEHHEEPTSSSGSSSREETTNSGRESSTPNGTSVPRNSSRTDGTWEDLFCDESLSSPEPPSSSEEPEEPPSSRSSPRQPPSSSAEEASSSQFTDEEYRSSSFTTPKTPPPFSRKRKFGGSRSSASSASSASFTSTPPKPKKNRETPVPTDFPIDLSDYLSHAVYSNKTVSCFAIYTTSDKAIELYDKIEKFKVDFKSRHACELGCILLFITLSKHRVSAIKNFCSTFCTISFLICKGVNKMPEMYNNLCKPPYKLLQENKPLLNYEFQEKEKEASCNWNLVAEFACEYELDDHFIILAHYLDFAKPFPCQKCENRSRLKPHKAHEAHHSNAKLFYESKSQKTICQQAADTVLAKRRLEMLEMTRTEMLCKKFKKHLERLRDLDTIDLLYYMGGVAWYCCLFEEFEKKLQKIIQLLTENIPKYRNIWFKGPINSGKTSFAAALIDLLEGKALNINCPSDKLPFELGCALDKFMVVFEDVKGQNSLNKDLQPGQGINNLDNLRDHLDGAVAVSLEKKHVNKKHQIFPPCIVTANDYFIPKTLIARFSYTLHFSPKANLRDSLDQNMEIRKRRILQSGTTLLLCLIWCLPDTTFKPCLQEEIKNWKQILQSEISYGKFCQMIENVEAGQDPLLNILIEEEGPEETEETQDSGTFSQ

SEQ ID NO: 46 - protein, 78 aa, Merkel cell polyomavirus
MDLVLNRKEREALCKLLEIAPNCYGNIPLMKAAFKRSCLKHHPDKGGNPVIMMELNTLWSKFQQNIHKLRSDFSMFDE

SEQ ID NO: 47 - protein, 131 aa, synthetic construct
EVQLLESGAEVKKPGASVRLSCKASGYTFTIYYMHWVRQAPGHGLEWMGIINPSGGRATYAEKLQGRLTMTRDTSTNTVYLELSSLRSEDTAVYYCARDRGGQNCTATRCPHYFFSGMDVWGQGTTVTVSS

SEQ ID NO: 48 - protein, 105 aa, synthetic construct
QYVLTQPPSVSVSPGQTARITCSGDELSKQYAYWYQQKPGQAPVLMIYKDTERPSGIPERFSGSSSGTTVTLSIIGVQAEDEADYYCQSADTTVFFGGGTRLTVL

SEQ ID NO: 49 - protein, 130 aa, synthetic construct
QLQLQESGPGLVKPSETLSLTCAVSVGSISSSSYYWGWIRQPPGKGLEWIGSISHSGNTYYNPSLKSRLTISVDTSKNQISLKVTSVTAADTAVYYCARQPQYFYDSSGHSLAYYGLDVWGRGTTVTVSS

SEQ ID NO: 50 - protein, 110 aa, synthetic construct
QFVLTQPASVSGSPGQSITISCTGASSDFGGYNSVSWYQQHSGKAPKLMIYDVHNRPSGVSNRFSGSKSGNTASLTISGLQADDEADYYCSSYTTSTFWVFGGGTKLTVL

SEQ ID NO: 51 - protein, 123 aa, synthetic construct
EVQLVESGGGVVQPGGSLRLSCAASGFNFSNYAMHWVRQAPGMGLEWVAVLSYDGSNEYYAGSVRGRFTISRDNSKNTLYLQIHSLRVEDTAVFYCARPRGSARRRFFYFDHWGQGALVTVSS

SEQ ID NO: 52 - protein, 112 aa, synthetic construct
QSVLTQPASVSGSPGQSITISCTGTINDVGGFDLVSWYQQLPGKAPKVLIYDVSDRPSGVSSRFSGSKSGNTASLTISGLQAEDEADYYCSSYTTSTSLLYVFGTGTKVTVL

SEQ ID NO: 53 - protein, 119 aa, synthetic construct
QVQLQESGAGLVRPSQTLSLTCAVSGDSISGGVYSWSWIRQTPEKGLEWIGYIDQSGSTYYSPSLKSRVTISVDTSENQFSLKLRSVTAADTAVYFCARASPMMVVLNWGQGTRVTVSS

SEQ ID NO: 54 - protein, 107 aa, synthetic construct
DIQMTQSPSTLSASVGDRVTITCRASQSVDTWLAWYQQKPGKAPKLLIHLASNLDSGVPTRFSGSGSGTEFTLTISSLQPDDFATYYCQQYHTHSGTFGQGTKVEIK

SEQ ID NO: 55 - protein, 122 aa, synthetic construct
EVQVVESGGGLVKPGGSLRLSCAGTGFTFNVYAMNWVRQAPGKGLEWVSSISPGGTSTYYADSVKGRFTISRDDAKNSLDLQMNSLRVEDTAIYYCARDSIFGVIMGGLDPWGQGTLVTVSS

SEQ ID NO: 56 - protein, 108 aa, synthetic construct
SYVLTQPPSVSVAPGQTASFTCGVRNHITDGVHWYLQKPGQAPVLVVYDDRDRPSGIPERFSGSRSRNTATLTISRVEAGDEADYYCQVWDRNRDQVIFGGGTKLTVL

SEQ ID NO: 57 - protein, 13 aa, synthetic construct
KASGYTFTIYYMH

SEQ ID NO: 58 - protein, 10 aa, synthetic construct
IINPSGGRAT

SEQ ID NO: 59 - protein, 24 aa, synthetic construct
ARDRGGQNCTATRCPHYFFSGMDV

SEQ ID NO: 60 - protein, 11 aa, synthetic construct
SGDELSKQYAY

SEQ ID NO: 61 - protein, 8 aa, synthetic construct
YKDTERPS

SEQ ID NO: 62 - protein, 8 aa, synthetic construct
QSADTTVF

SEQ ID NO: 63 - protein, 5 aa, synthetic construct
IYYMH

SEQ ID NO: 64 - protein, 17 aa, synthetic construct
IINPSGGRATYAEKLQG

SEQ ID NO: 65 - protein, 22 aa, synthetic construct
DRGGQNCTATRCPHYFFSGMDV

SEQ ID NO: 66 - protein, 7 aa, synthetic construct
KDTERPS

SEQ ID NO: 67 - protein, 8 aa, synthetic construct
GYTFTIYY

SEQ ID NO: 68 - protein, 8 aa, synthetic construct
INPSGGRA

SEQ ID NO: 69 - protein, 6 aa, synthetic construct
ELSKQY

SEQ ID NO: 70 - protein, 7 aa, synthetic construct
GYTFTIY

SEQ ID NO: 71 - protein, 13 aa, synthetic construct
WMGIINPSGGRAT

SEQ ID NO: 72 - protein, 6 aa, synthetic construct
TIYYMH

SEQ ID NO: 73 - protein, 23 aa, synthetic construct
ARDRGGQNCTATRCPHYFFSGMD

SEQ ID NO: 74 - protein, 7 aa, synthetic construct
KQYAYWY

SEQ ID NO: 75 - protein, 10 aa, synthetic construct
LMIYKDTERP

SEQ ID NO: 76 - protein, 7 aa, synthetic construct
QSADTTV

SEQ ID NO: 77-78 - skipped (2 entries)

SEQ ID NO: 79 - protein, 15 aa, synthetic construct
AVSVGSISSSSYYWG

SEQ ID NO: 80 - protein, 9 aa, synthetic construct
SISHSGNTY

SEQ ID NO: 81 - protein, 22 aa, synthetic construct
ARQPQYFYDSSGHSLAYYGLDV

SEQ ID NO: 82 - protein, 14 aa, synthetic construct
TGASSDFGGYNSVS

SEQ ID NO: 83 - protein, 8 aa, synthetic construct
YDVHNRPS

SEQ ID NO: 84 - protein, 10 aa, synthetic construct
SSYTTSTFWV

SEQ ID NO: 85 - protein, 7 aa, synthetic construct
SSSYYWG

SEQ ID NO: 86 - protein, 16 aa, synthetic construct
SISHSGNTYYNPSLKS

SEQ ID NO: 87 - protein, 20 aa, synthetic construct
QPQYFYDSSGHSLAYYGLDV

SEQ ID NO: 88 - protein, 7 aa, synthetic construct
DVHNRPS

SEQ ID NO: 89 - protein, 10 aa, synthetic construct
VGSISSSSYY

SEQ ID NO: 90 - protein, 7 aa, synthetic construct
ISHSGNT

SEQ ID NO: 91 - protein, 9 aa, synthetic construct
SSDFGGYNS

SEQ ID NO: 92 - protein, 9 aa, synthetic construct
VGSISSSSY

SEQ ID NO: 93 - protein, 5 aa, synthetic construct
SHSGN

SEQ ID NO: 94 - protein, 8 aa, synthetic construct
SSSSYYWG

SEQ ID NO: 95 - protein, 12 aa, synthetic construct
WIGSISHSGNTY

SEQ ID NO: 96 - protein, 21 aa, synthetic construct
ARQPQYFYDSSGHSLAYYGLD

SEQ ID NO: 97 - protein, 7 aa, synthetic construct
YNSVSWY

SEQ ID NO: 98 - protein, 10 aa, synthetic construct
LMIYDVHNRP

SEQ ID NO: 99 - protein, 9 aa, synthetic construct
SSYTTSTFW

SEQ ID NO: 100-101 - skipped (2 entries)

SEQ ID NO: 102 - protein, 13 aa, synthetic construct
AASGFNFSNYAMH

SEQ ID NO: 103 - protein, 10 aa, synthetic construct
VLSYDGSNEY

SEQ ID NO: 104 - protein, 16 aa, synthetic construct
ARPRGSARRRFFYFDH

SEQ ID NO: 105 - protein, 14 aa, synthetic construct
TGTINDVGGFDLVS

SEQ ID NO: 106 - protein, 8 aa, synthetic construct
YDVSDRPS

SEQ ID NO: 107 - protein, 12 aa, synthetic construct
SSYTTSTSLLYV

SEQ ID NO: 108 - protein, 5 aa, synthetic construct
NYAMH

SEQ ID NO: 109 - protein, 17 aa, synthetic construct
VLSYDGSNEYYAGSVRG

SEQ ID NO: 110 - protein, 14 aa, synthetic construct
PRGSARRRFFYFDH

SEQ ID NO: 111 - protein, 7 aa, synthetic construct
DVSDRPS

SEQ ID NO: 112 - protein, 8 aa, synthetic construct
GFNFSNYA

SEQ ID NO: 113 - protein, 8 aa, synthetic construct
LSYDGSNE

SEQ ID NO: 114 - protein, 9 aa, synthetic construct
INDVGGFDL

SEQ ID NO: 115 - protein, 7 aa, synthetic construct
GFNFSNY

SEQ ID NO: 116 - protein, 6 aa, synthetic construct
SYDGSN

SEQ ID NO: 117 - protein, 6 aa, synthetic construct
SNYAMH

SEQ ID NO: 118 - protein, 13 aa, synthetic construct
WVAVLSYDGSNEY

SEQ ID NO: 119 - protein, 15 aa, synthetic construct
ARPRGSARRRFFYFD

SEQ ID NO: 120 - protein, 10 aa, synthetic construct
VGGFDLVSWY

SEQ ID NO: 121 - protein, 10 aa, synthetic construct
VLIYDVSDRP

SEQ ID NO: 122 - protein, 11 aa, synthetic construct
SSYTTSTSLLY

SEQ ID NO: 123-124 - skipped (2 entries)

SEQ ID NO: 125 - protein, 15 aa, synthetic construct
AVSGDSISGGVYSWS

SEQ ID NO: 126 - protein, 9 aa, synthetic construct
YIDQSGSTY

SEQ ID NO: 127 - protein, 11 aa, synthetic construct
ARASPMMVVLN

SEQ ID NO: 128 - protein, 11 aa, synthetic construct
RASQSVDTWLA

SEQ ID NO: 129 - protein, 8 aa, synthetic construct
HLASNLDS

SEQ ID NO: 130 - protein, 9 aa, synthetic construct
QQYHTHSGT

SEQ ID NO: 131 - protein, 7 aa, synthetic construct
GGVYSWS

SEQ ID NO: 132 - protein, 16 aa, synthetic construct
YIDQSGSTYYSPSLKS

SEQ ID NO: 133 - protein, 9 aa, synthetic construct
ASPMMVVLN

SEQ ID NO: 134 - protein, 7 aa, synthetic construct
LASNLDS

SEQ ID NO: 135 - protein, 10 aa, synthetic construct
GDSISGGVYS

SEQ ID NO: 136 - protein, 7 aa, synthetic construct
IDQSGST

SEQ ID NO: 137 - protein, 6 aa, synthetic construct
QSVDTW

SEQ ID NO: 138 - protein, 9 aa, synthetic construct
GDSISGGVY

SEQ ID NO: 139 - protein, 5 aa, synthetic construct
DQSGS

SEQ ID NO: 140 - protein, 8 aa, synthetic construct
SGGVYSWS

SEQ ID NO: 141 - protein, 12 aa, synthetic construct
WIGYIDQSGSTY

SEQ ID NO: 142 - protein, 10 aa, synthetic construct
ARASPMMVVL

SEQ ID NO: 143 - protein, 7 aa, synthetic construct
DTWLAWY

SEQ ID NO: 144 - protein, 10 aa, synthetic construct
LLIHLASNLD

SEQ ID NO: 145 - protein, 8 aa, synthetic construct
QQYHTHSG

SEQ ID NO: 146-147 - skipped (2 entries)

SEQ ID NO: 148 - protein, 13 aa, synthetic construct
AGTGFTFNVYAMN

SEQ ID NO: 149 - protein, 10 aa, synthetic construct
SISPGGTSTY

SEQ ID NO: 150 - protein, 15 aa, synthetic construct
ARDSIFGVIMGGLDP

SEQ ID NO: 151 - protein, 11 aa, synthetic construct
GVRNHITDGVH

SEQ ID NO: 152 - protein, 8 aa, synthetic construct
YDDRDRPS

SEQ ID NO: 153 - protein, 11 aa, synthetic construct
QVWDRNRDQVI

SEQ ID NO: 154 - protein, 5 aa, synthetic construct
VYAMN

SEQ ID NO: 155 - protein, 17 aa, synthetic construct
SISPGGTSTYYADSVKG

SEQ ID NO: 156 - protein, 13 aa, synthetic construct
DSIFGVIMGGLDP

SEQ ID NO: 157 - protein, 7 aa, synthetic construct
DDRDRPS

SEQ ID NO: 158 - protein, 8 aa, synthetic construct
GFTFNVYA

SEQ ID NO: 159 - protein, 8 aa, synthetic construct
ISPGGTST

SEQ ID NO: 160 - protein, 6 aa, synthetic construct
NHITDG

SEQ ID NO: 161 - protein, 7 aa, synthetic construct
GFTFNVY

SEQ ID NO: 162 - protein, 6 aa, synthetic construct
SPGGTS

SEQ ID NO: 163 - protein, 6 aa, synthetic construct
NVYAMN

SEQ ID NO: 164 - protein, 13 aa, synthetic construct
WVSSISPGGTSTY

SEQ ID NO: 165 - protein, 14 aa, synthetic construct
ARDSIFGVIMGGLD

SEQ ID NO: 166 - protein, 7 aa, synthetic construct
TDGVHWY

SEQ ID NO: 167 - protein, 10 aa, synthetic construct
LVVYDDRDRP

SEQ ID NO: 168 - protein, 10 aa, synthetic construct
QVWDRNRDQV

SEQ ID NO: 169 - protein, 120 aa, synthetic construct
GVQLVESGGGFVQPGGSLRLSCAASGVTFSRFDMNWVRQAPGKGLEWVSYISSSGTTIYYADSVKGRFTISRDNAKNSLYLRLNSLRADDTAVYYCARDDYTGWYYFDSWGQGTLVTVSS

SEQ ID NO: 170 - protein, 109 aa, synthetic construct
QSVLTQPPSVSGAPGQRVTISCTGSSSNIGAGFDVHWYQQFPGTAPKLLIYGNSNRPSGVPDRFSGSKSGTSASLAITGLQAEDEADYYCQSYDSSLYVFGTGTKVTVL

SEQ ID NO: 171 - protein, 120 aa, synthetic construct
QVTLRESGPALVKPTQTLTLTCTFSGFSLSTSGMCVSWIRQPPGKALEWLARIDWDDDKYYNTSLKTRLTISKVTSKNRVVLTMTNMDPVDTATYYCARQELVGAWFDPWGQGTLVTVSS

SEQ ID NO: 172 - protein, 111 aa, synthetic construct
QSALTQPASVSGSPGQSITVSCTGTSGDVGGYNYVSWYQQHPGKAPKLIIYEVSNRPSGVSNRFSGSKSGNTASLTISGLQAEDEAEYYCISYTSSSISCVFGTGTKVTVL

SEQ ID NO: 173 - protein, 121 aa, synthetic construct
QVQLVESGGGVVQPGRSLRLSCAASGFTFSSYGMHWVRQAPGKGLEWVAVISYDGSTKYYADSVKGRFTISRDNSKNTLYLQMNSLRAADTAVYYCAKDGTAVLVDRLDYWGQGTLVTVSS

SEQ ID NO: 174 - protein, 107 aa, synthetic construct
AIQLTQSPSSLSASVGDRVTITCRASQGISSYLAWYQQNPGKAPKLLIYAASTLQSGVPSRFSGSGSGTDFTLTISSLQPEDFATYYCQQLNSYPLTFGGGTKVEIK

SEQ ID NO: 175 - protein, 114 aa, synthetic construct
EVQLVQSGAEMKKPGESLRISCAASGYDFDTYWIAWVRQMPGKGLDWMGVIYPDDSDTKYSPSFQGQVTISVDKSINTAYLQWTSLKASDTAIYYCARATGDYWGQGTLVTVSS

SEQ ID NO: 176 - protein, 107 aa, synthetic construct
DIQMTQSPSALSASVGDRVTISCRASQNVSTWLTWYQQKPGAAPKRLIYEASTLQSGVPSRFSGRGSGTEFFLTISSLQPDDFATYFCQQYRSYSRTFGQGTKVEIR

SEQ ID NO: 177 - protein, 120 aa, synthetic construct
QVTLRESGPALVKPTQTLTLTCTFSGFSLSTSGMCVSWIRQPPGKALEWLARIDWDDDKYYNTSLKTRLTISKDTSKNRVVLTMTNMDPVDTATYYCARQELVGAWFDPWGQGTLVTVSS

SEQ ID NO: 178 - protein, 111 aa, synthetic construct; identical to SEQ ID NO: 172

SEQ ID NO: 179 - protein, 114 aa, synthetic construct
EVQLVQSGPEAKKPGESLRISCAGSGYDFNTYWIAWVRQMPGKGLDWMGVIYPDDSDTKYSPSFQGQVTISVDKSITTAYLQWSSLKASDTAMYYCARATGDYWGQGTLVTVSS

SEQ ID NO: 180 - protein, 107 aa, synthetic construct
DIQMTQSPSALSASVGDRVTISCRASQNVSRWLTWYQQKPGAAPKRLIYEASTLQSGVPSRFSGRGSGTEFFLTISSLQPDDFATYFCQQYRSYSRTFGQGTKVEIR

SEQ ID NO: 181 - protein, 122 aa, synthetic construct
QVQLVQSGAEVKRPGSSVKVSCKASGATFSSYAISWVRQAPGQGLEWLGRVTPIIDTTNYAQKFQGRVTITADESTSTAYMELSGLRSEDTAIYYCATRGTYYDFWPSMDVWGQGTTVTVSS

SEQ ID NO: 182 - protein, 107 aa, synthetic construct
DIQMTQSPSSLSASVGDRVTITCRASQDIRNYLAWFQQKPGKAPKSLIYAASSLHSGVPSKFSGSGSGTDFTLTISGLQPEDFATYYCQQYNSYPLTFGGGTRVEIK

SEQ ID NO: 183 - protein, 119 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCASMSGSYYGAFDYWGQGTLVTVSS

SEQ ID NO: 184 - protein, 108 aa, synthetic construct
SYELTQPPSVSVSPGQTARITCSGDALPKQYAYWYQQKPGQAPVLVIYKDSERPSGIPERFSGSSSGTTVTLTISGVQAEDEADYYCQSADSSGTYYVFGTGTKVTVL

SEQ ID NO: 185 - protein, 118 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCASIIAALFDYWGQGTLVTVSS

SEQ ID NO: 186 - protein, 105 aa, synthetic construct
YELTQPSSVSVSPGQTARITCSGDVLAKKYARWFQQKPGQAPVLVIYKDSERPSGIPERFSGSSSGTTVTLTISGAQVEDEADYYCYSAADNNWVFGGGTKLTVL

SEQ ID NO: 187 - protein, 121 aa, synthetic construct
QVQLVKSGGGVVQPERSLRLSCAASGFTFKHYSMHWVRQAPGKGLEWVAHISYDGSKTNYADSVKGRFSISRDNSENTLYLQMNSLKTDDTALYYCVRDRTTGHDFGFDYWGPGTQVIVSS

SEQ ID NO: 188 - protein, 107 aa, synthetic construct
HELTQPPSVSVSPGLTARITCSGDALSKKYAYWYQQKSGQAPVLVMFKDNERPAGVPERFSGSSSGTTVTLTISGVQAEDEADYYCQSTDSDTSYVIFGGGTKLTVL

SEQ ID NO: 189 - protein, 120 aa, synthetic construct
QVQLVQSGAEVKKPGSSLKVSCKTSGGFFSSYRINWVRQAPGQGLEWMGGLVPIFETAKYAQKFQGRVSITADESTSTVYMEVTSLTSDDTAVYYCARGSGSGLTYFQTWGQGSLVTVSS

SEQ ID NO: 190 - protein, 107 aa, synthetic construct
EIVLTQSPGTLSLSPGERATLSCRASQSITTYYLAWYQQKPGQAPRLLIYGASSRATGIPDRFSGSGSGTDFTLTISRLDPEDFAVYYCQQYDSSPTFGQGTKVEVK

SEQ ID NO: 191 - protein, 118 aa, synthetic construct
QLQLQESGPGLVKPSETLPLSCTVSGGSITSISHYWGWIRQPPGKGLEWIGSMYYSGTTYYNPSLKSRVTMSVDTSKNQFSLKLSSVTAADTAVYYCARHDVSRFDPWGQGTLVTVSS

SEQ ID NO: 192 - protein, 109 aa, synthetic construct
QSALTQPASVSGSPGQSITISCTGTSSDVGGYNYVSWYQHHPGKAPKLMIYDVTNRPSGVSNRFSGSKSGNTASLTISGLQAEDEADYYCSSYTTSTWVFGGGTKLTVL

SEQ ID NO: 193 - protein, 120 aa, synthetic construct
RGQLVESGGGVVQPGRALRLSCVASGFTFSNFDMHWIRQAPGKGLEWVAVISYDESKRQHADSVKGRFTISRDKSKNTVYLQMNSLRAEDTAVYYCATESTQYFGALDTWGQGTLVTVSS

SEQ ID NO: 194 - protein, 107 aa, synthetic construct
SYELTQPPSVSVSPGQTARITCSGDGLADQYVYWYRQRPGQAPALVMYKDSERPSGIPERFSGASSGTQVTLTISGVQAEDEAEYFCQSTDRSGSYLFGGGTTLTVL

SEQ ID NO: 195 - protein, 121 aa, synthetic construct
QVQLAESGGGVVQPGRSLRLSCAASGFNFNFYTMHWVRQAPGKGLEWVALVFYDGSKTYYADSVKGRSTISRDNSRRTLFLEINALRPEDTAMYYCVRDRTTGHDYGMDVWGQGTTVIVSS

SEQ ID NO: 196 - protein, 108 aa, synthetic construct
SYELTQPPSVSVSPGQTATIACSGAGLANLYTYWYQQKPGQAPILLIYKDTERPSGIPERFSGSNSGTTVTLTISGVQAGDEADYYCQSADSSRSSVIFGGGTKLTVL

SEQ ID NO: 197 - protein, 120 aa, synthetic construct; identical to SEQ ID NO: 189

SEQ ID NO: 198 - protein, 107 aa, synthetic construct; identical to SEQ ID NO: 190

SEQ ID NO: 199 - protein, 124 aa, synthetic construct
QVTLRQSGPALVKSTQTLTLTCTFSGFSLSTGGVCINWIRQPPGKALEWLARIDWDNDKYYRPSLKTRLTISKDTSKNQVVLTMTNMDPVDTATYYCARMTGLYSDNWKGFDIWGQGTMVTVSS

SEQ ID NO: 200 - protein, 109 aa, synthetic construct
EIVLTQSPGTLSLSPGERATLSCRASQSVDSTYLAWYQQKPGQAPRLLIYSASRRATGIPDRFSGSGSGTDFTLTISRLEPEDFAFYYCQQYGSAPGYTFGQGTKLEIK

SEQ ID NO: 201 - protein, 124 aa, synthetic construct
QVTLRQSGPALVKPTQTLTLTCTVSGFSLNTAEVCWNWIRQPPGKALEWLARIDWDDDKYIRPSLKTRLTISKDTSKNQVVLTMTNMDPVDTATYYCARMTGLYTDNWKGFDMWGQGTMVTVSS

SEQ ID NO: 202 - protein, 109 aa, synthetic construct
EIVLTQSPDTLSLSPGERATLSCRASQTIDSTYFAWYQQKPGQAPRLLIYSASRRATGVPDRFSGSASGTDFTLTISRLEPEDFAFYYCQQYGHAPGYTFGQGTKLDIK

SEQ ID NO: 203 - protein, 126 aa, synthetic construct
QVQLVQSGAEVKKPGSSVKVSCKAPGGTFSRYAISWVRQAPGQGLEWMGGIIPMFDITNYAQKLQDRIRITADESTSTAYMELSSLRSEDTALYYCATESCSGGNCYWDHDAFDIWGQGTMVTVSS

SEQ ID NO: 204 - protein, 109 aa, synthetic construct
EIVLTQSPGTLSLSPGERATLSCRASQSLSSSYLAWYQQKAGQAPRLLIYGASTRATGIPDRFSGSGSGTDFTLTISRLEPEDFAVYYCQQYGRSLPRTFGQGTKVEIK

SEQ ID NO: 205 - protein, 126 aa, synthetic construct
QVQLVQSGAEVKKPGSSVKVSCKASGGTFSSYAISWVRQAPGQGLEWMGGIIPIFDIANYAQKFQGRVTITADESTSTAFMELSSLRSEDTAVYYCASENCGGGNCYWEHDAFDIWGQGTRVTVSS

SEQ ID NO: 206 - protein, 109 aa, synthetic construct
EIVLTQSPDTLSLSPGERATLSCRASQSVSGGDLAWYQQKPGQAPRLLMYGASIRATGIPDRFSGSGSGTDFTLTISRLEPEDFVVYYCQHYGNSPPRTFGQGTKVEIK

SEQ ID NO: 207 - protein, 118 aa, synthetic construct
EVQLVQSAAEVKKPGESLKISCQTSGYSFASFWIGWVRQMPGKGLEWMGIIYPSDSDTTYSPTFQGLVTISVDKSSRTAYLQWSSLKASDTAMYYCAKHGVGAAIDHWGQGTLVTVSS

SEQ ID NO: 208 - protein, 107 aa, synthetic construct
DIQMTQSPSSLSASVGDRVTITCRTSQVISGYLNWYQQRPGKVPKLLIYTTSTLQPGVPSRFSGSGSGTDFTLSISNLQPEDFATYYCQQSYSSPRTFGQGTKLEIK

SEQ ID NO: 209 - protein, 119 aa, synthetic construct
QVQLQESGPGLVKPSQTLSLTCTVSGGSIGGGPYHWSWIRQPAGKGLEWIGHVETSGDTKTNPSFRSRVTTSLDTATNQLSLKVHSVTAADTAVYYCARGTAMDAFDIWGQGTVVIVSS

SEQ ID NO: 210 - protein, 107 aa, synthetic construct
DIQMTQSPSSVSASVGDRVTITCRASQDISNWLAWYQQRPGKAPNLLIYVASGLQSGVPSRFSGSGSGTEFTLTISTLQPEDSATYYCQQGNSFPLTFGGGTRVEIN

SEQ ID NO: 211 - protein, 117 aa, synthetic construct
QVQLVESGGGVVQPGRSLRLSCAASGFSFSGYGMHWVRQAPGKGLEWVAFIWYDGSSKKYGDSVKGRFIISRDNSKNTVYLQMNSLRADDTAVYYCARDVLGELPDWGQGTLVTVSS

SEQ ID NO: 212 - protein, 107 aa, synthetic construct
DIQMTQSPSSLSASVGDRVTITCRASQGISNSLGWFQQKPGKAPKSLIYAASSLQSGVPSKFSGSGSGTNFSLTISSLQPEDFATYYCQQYNTYPLTFGGGTKVEIK

SEQ ID NO: 213 - protein, 120 aa, synthetic construct
RVQLVESGGGVVQPGRSLRLSCATSGFTFSNYGLHWVRQAPGKGLEWLAAIWFDGRTFYADSVKDRFSISRDNSKNTVYLQINSLRVEDTAVYYCARETWSYAGGVFDYWGQGTLVIVSS

SEQ ID NO: 214 - protein, 111 aa, synthetic construct
QPVLTQSPSASAPLGASVKLVCTLSSGHSGHAIAWHQQQAEKGPRFLMKVHSDGSHNKGDGIPDRFSGSVSGAERYLSISSLQSEDEADYYCQTWDTGLWVFGGGTKLTVL

SEQ ID NO: 215 - protein, 122 aa, synthetic construct
QVQLVQSGAEVKKPGSSVKVSCKTSGGTFSSATLSWVRQAPGQGLEWMGGITPMFTTANYAQKFQGRVTISADESTNTAYMKLSSLRSEDTAVYYCARQFGHESRDSYYLYWGQGTLVTVSS

SEQ ID NO: 216 - protein, 107 aa, synthetic construct
EIVLTQSPGTLSLSPGERATLSCRASQTLSSSYLAWYQQKPGQAPRLLIYGASSRVAGVPVRFRGSGSGTDFTLTISRLEPEDFAVYYCQQYGSSRTFGQGTKVEIK

SEQ ID NO: 217 - protein, 121 aa, synthetic construct
QVQLQESGPGLVKPSETLALTCTVSGYSISSGYYWGWIRQPPGKGLEWIGSIYHSGSTYYNPSLNSRVTISVDTSKNQFSLRLSSVTAADTAVYYCARSARAVVMDAFHIWSQGTVVTVSS

SEQ ID NO: 218 - protein, 111 aa, synthetic construct
NFMLTQPHSVSESPGKTVTISCTGSSGSIASNYVQWYQQRPGSAPTTVIYEDNQRPSGVPDRFSGSIDSSSNSASLTISGLKTEDEADYYCQSYDNIHPWVFGGGTKVTVL

SEQ ID NO: 219 - protein, 121 aa, synthetic construct; identical to SEQ ID NO: 217

SEQ ID NO: 220 - protein, 109 aa, synthetic construct
SYVLTQPPSVSVAPGKTARITCGGNNIGSKSVHWYQQKPGQAPVLVIYYDSDRPSGIPERFSGSSSGNTATLTISRVEAGDEADYYCQVWDGSTDHFYVFGTGTKVTVL

SEQ ID NO: 221 - protein, 125 aa, synthetic construct
QVQLQESGPGLVKPSQTLSLSCTVSGGSISSGEYYWSWIRQPPGKGLEWIGYISYAGNTYYNPSLKSRLNISLDKSKNQFSLKLTSVTAADTAIYYCARDFVGTNWYDRYAFHIWGQGTMVTVSS

SEQ ID NO: 222 - protein, 123 aa, synthetic construct
LSLLTQGTGSWAQSALTQPRSVSGSPGHSVTISCTGTSSDVGGYHYVSWYQQLPGKAPKLILYDVTKRPSGVPDRFSGSKSGNTASLTISGLQAEDEADYFCCSYAGSYAFWVFGGGTKLTVL

SEQ ID NO: 223 - protein, 123 aa, synthetic construct
CEVQLVESGGGLVRPGGSLRLSCGASGFNFIDYAMYWVRQAPGKALEYVSSITNDAKSTFYGNSVKGRFTISRDNSKNTVFLQMGSLRVEDMAVYYCARGRSHYYDSSGFDYWGQGALVTVSP

SEQ ID NO: 224 - protein, 107 aa, synthetic construct
SYVLTQPPSVSVAPGQTARITCGGDSLGSKSVHWYQQKPGQAPVLVVFDDSDRPSGIPERFSGSNSGNRGTLTISRVEAGDEAVYYCQVWDAGALWVFGGGTELTVL

SEQ ID NO: 225 - protein, 124 aa, synthetic construct
QLQLQESGPGLVKPSETLSLTCSVSGDSISTTSYYWDWFRQPPGKGLEWIGSINYSGNTYYSPLLKSRVTIASDTSKNQFFLRLTSVTAADTAVYYCARRGVPAGMVPSWIDPWGQGTLVAVSS

SEQ ID NO: 226 - protein, 107 aa, synthetic construct
DIQMTQSPSSLSASVGDRVTITCQASEDIRNYLNWYQQKPGKAPKVLIYDASNLKTGVPSRFSGSGSRTHFTFTISSLQPEDIATYYCQQYDTVPLTFGQGTKLEIK

SEQ ID NO: 227 - protein, 114 aa, synthetic construct
EVQLVQSGAEVKKPGESLKISCKGSGYHFPTYWIAWVRQMPGKGLEWMGVIYPRDSDTKYSPSFQGQVTISVDKSITTAYLQWNSLKASDTAMYYCARATGDYWGQGTLVTVSS

SEQ ID NO: 228 - protein, 106 aa, synthetic construct
DIQMTQSPSTLSASVGDRVTITCRAGENVSTWLTWYQQKPGRAPERLMYKASTLETGVPSRFSGRGSGTEFILTISSLQPDDFATYYCQQYKSYSRTFGQGTKVEI

SEQ ID NO: 229 - protein, 114 aa, synthetic construct
EVQLVQSGAEVKKPGESLKISCQGFGYSFPTYWIAWVRQMPGKGLEWMGIIYPDDSDTTYSPSFQGQVTFSADKSTNTAYLQWDSLKVSDSALYYCARGMGDHWGQGTLITVSS

SEQ ID NO: 230 - protein, 112 aa, synthetic construct
DVVLTQTPLSLSVTPGQPASISCKSSQSLLFGDGATYLYWFLQRPGQSPHLLISKVSSRFSGVPDRFNGSGSGTDFTLKISRVEAEDFGVYYCMQALHMPRTFGQGTKVEIK

SEQ ID NO: 231 - protein, 121 aa, synthetic construct
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCARDRKIVVRRGGLDIWGQGTMVTVSS

SEQ ID NO: 232 - protein, 107 aa, synthetic construct
AIRMTQSPSSFSASTGDRVTITCRASQGISSYLAWYQQKPGKAPKLLIYAASTLQSGVPSRFSGSGSGTDFTLTISCLQSEDFATYYCQQYYSYPHTFGQGTRLEIK

SEQ ID NO: 233 - protein, 118 aa, synthetic construct
EVQLVESGGGLVQPGGSLRLSCAASGFTFSSYSMNWVRQAPGKGLEWVSYISSSSSTIYYADSVKGRFTISRDNAKNSLYLQMNSLRDEDTAVYYCARDGAAAGVNPWGQGTLVTVSS

SEQ ID NO: 234 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSFPWTFGQGTKVEIK

SEQ ID NO: 235 - protein, 128 aa, synthetic construct
EVQLVESGGGLVQPGGSLKLSCAASGFTFSGSAMHWVRQASGKGLEWVGRIRSKANSYATAYAASVKGRFTISRDDSKNTAYLQMNSLKTEDTAVYYCTRLTDIFRLLGSNFYGMDVWGQGTTVTVSS

SEQ ID NO: 236 - protein, 108 aa, synthetic construct
SYELTQPPSVSVSPGQTARITCSGDALPKQYAYWYQQKPGQAPVLVIYKDSERPSGIPERFSGSSSGTTVTLTISGVQAEDEADYYCQSADSSGTYVVFGGGTKLTVL

SEQ ID NO: 237 - protein, 125 aa, synthetic construct
EVQLLESGGGLVQPGGSLRLSCAASRFTFSRYAMTWVRQAPGKGLEWVSAISSTGDNPYYADSVKGRFTISRDNSKNILYLQMNSLRAEDTAVYYCAKDQGGADNNGYYYNFDYWGQGTLVTVSS

SEQ ID NO: 238 - protein, 108 aa, synthetic construct
SYVLTQPSSVSVAPGKTARITCGGNDIGSKSVHWYQQKPGQAPVQVIYYDSDRPSGIPERFSGSNSGNTATLTISRVEAGDEADYYCQVWDSSSDHVVFGGGTKLTVL

SEQ ID NO: 239 - protein, 121 aa, synthetic construct
QVQLLESGGGLVQPGGSLRLSCAASGFSFSTYAMNWVRQAPGKGLEWVSLFSGSGGDTYYADSVKGRFTISRDNSKNTLYLQMKNLRVEDTAIYYCAKDLGVMFFGAFDIWGQGTMVTVSS

SEQ ID NO: 240 - protein, 107 aa, synthetic construct
DIQMTQSPSILSASVGDRVTITCRASQSISSWLAWYQQKPGKAPKLLIYKASSLQSGVPSRFSGSGSGTEFTLTISSLQPDDFATYYCQQYNDYSVTFGQGTKLEIK

SEQ ID NO: 241 - protein, 123 aa, synthetic construct
EVQLVESGGGLVQPGGSLRLSCAASGFTFTNYWMHWVRQAPGKGLVWVSRLNSDGSITGYADSVKGRFTISRDNPKNTLYLQMNSLRAEDTAIYYCARVGLRTLPAIENFDYWGQGTLVTVSS

SEQ ID NO: 242 - protein, 107 aa, synthetic construct
DIQMTQSPSSLSASVGDRVTITCRASQSISIYLNWYQQKPGKPPKLLIYAASSLQSGVPSRFSGSGSGTDFTLTISSLHPEDFATYYCQQSYRAVLTFGGGTKVEIK

SEQ ID NO: 243 - protein, 121 aa, synthetic construct
EVQLVESGGGLVQPGGSRRLSCAASGFIFSYYWMSWVRQAPGKGLEWVANIKEDGSEKYYVDSVKGRFTISRDNAKTSLFLQMNTLRAEDTAVYYCARAPLRNFDWPLDVWGQGALVTVSS

SEQ ID NO: 244 - protein, 108 aa, synthetic construct
QSALTQPRSVSGSPGQSLSISCTGTSSDVGANDYVSWYQQHPGRAPKLIIYDVSKRTSGVPHRFSGSKSGNTASLTISGLQAEDEADYYCCSYGGRYVFGTGTEVTVL

SEQ ID NO: 245 - protein, 115 aa, synthetic construct
EVQLVQSGAEVKKPGESLKISCKGSGYKFSSYWIAWVCQMPGKGLEWMGIIYPDDSDTTYSPSFQGQVTISADKSINTAYLQWNSLKASDTAMYYCVRGGSAPHWGQGTLVTVSS

SEQ ID NO: 246 - protein, 113 aa, synthetic construct
DIVMTQSPDSLAVSLGERATINCKSSQSVLYSSNNKNYLTWYQQKPGQPPKLLIYWASTRESGVPDRFSGSGSGTDFTLTINSLQAEDVAVYYCQQHYTTPWTFGQGTKVEIQ

SEQ ID NO: 247 - protein, 122 aa, synthetic construct
QVQLVQSGAEVKKPGASVKVSCKASGYTFNTFYMHWVRQAPGQGLEWVGVINPSRGRTIYAQKFQGRVTMTRDTSTSTVYMELSSLRSEDTAVYYCARDAGLGPYYYYMDVWGKGTTVTVSS

SEQ ID NO: 248 - protein, 108 aa, synthetic construct
SYELTQPPSVSVSPGQTARITCSGDGLTKQYAFWYQQKPGQAPVLVIYKDSERPSGIPERFSGSSSGTTVTLTISGVQAEDEADYYCQSADRSDIYVVFGGGTKLTVL

SEQ ID NO: 249 - skipped

SEQ ID NO: 250 - protein, 13 aa, synthetic construct
AASGVTFSRFDMN

SEQ ID NO: 251 - protein, 10 aa, synthetic construct
YISSSGTTIY

SEQ ID NO: 252 - protein, 13 aa, synthetic construct
ARDDYTGWYYFDS

SEQ ID NO: 253 - protein, 14 aa, synthetic construct
TGSSSNIGAGFDVH

SEQ ID NO: 254 - protein, 8 aa, synthetic construct
YGNSNRPS

SEQ ID NO: 255 - protein, 9 aa, synthetic construct
QSYDSSLYV

SEQ ID NO: 256 - protein, 5 aa, synthetic construct
RFDMN

SEQ ID NO: 257 - protein, 17 aa, synthetic construct
YISSSGTTIYYADSVKG

SEQ ID NO: 258 - protein, 11 aa, synthetic construct
DDYTGWYYFDS

SEQ ID NO: 259 - protein, 7 aa, synthetic construct
GNSNRPS

SEQ ID NO: 260 - protein, 8 aa, synthetic construct
GVTFSRFD

SEQ ID NO: 261 - protein, 8 aa, synthetic construct
ISSSGTTI

SEQ ID NO: 262 - protein, 9 aa, synthetic construct
SSNIGAGFD

SEQ ID NO: 263 - protein, 7 aa, synthetic construct
GVTFSRF

SEQ ID NO: 264 - protein, 6 aa, synthetic construct
SSSGTT

SEQ ID NO: 265 - protein, 6 aa, synthetic construct
SRFDMN

SEQ ID NO: 266 - protein, 13 aa, synthetic construct
WVSYISSSGTTIY

SEQ ID NO: 267 - protein, 12 aa, synthetic construct
ARDDYTGWYYFD

SEQ ID NO: 268 - protein, 10 aa, synthetic construct
IGAGFDVHWY

SEQ ID NO: 269 - protein, 10 aa, synthetic construct
LLIYGNSNRP

SEQ ID NO: 270 - protein, 8 aa, synthetic construct
QSYDSSLY

SEQ ID NO: 271 - protein, 15 aa, synthetic construct
TFSGFSLSTSGMCVS

SEQ ID NO: 272 - protein, 9 aa, synthetic construct
RIDWDDDKY

SEQ ID NO: 273 - protein, 12 aa, synthetic construct
ARQELVGAWFDP

SEQ ID NO: 274 - protein, 14 aa, synthetic construct
TGTSGDVGGYNYVS

SEQ ID NO: 275 - protein, 8 aa, synthetic construct
YEVSNRPS

SEQ ID NO: 276 - protein, 11 aa, synthetic construct
ISYTSSSISCV

SEQ ID NO: 277 - protein, 7 aa, synthetic construct
TSGMCVS

SEQ ID NO: 278 - protein, 16 aa, synthetic construct
RIDWDDDKYYNTSLKT

SEQ ID NO: 279 - protein, 10 aa, synthetic construct
QELVGAWFDP

SEQ ID NO: 280 - protein, 7 aa, synthetic construct
EVSNRPS

SEQ ID NO: 281 - protein, 10 aa, synthetic construct
GFSLSTSGMC

SEQ ID NO: 282 - protein, 7 aa, synthetic construct
IDWDDDK

SEQ ID NO: 283 - protein, 9 aa, synthetic construct
SGDVGGYNY

SEQ ID NO: 284 - protein, 9 aa, synthetic construct
GFSLSTSGM

SEQ ID NO: 285 - protein, 9 aa, synthetic construct
GFSLSTSGM

SEQ ID NO: 286 - protein, 8 aa, synthetic construct
STSGMCVS

SEQ ID NO: 287 - protein, 12 aa, synthetic construct
WLARIDWDDDKY

SEQ ID NO: 288 - protein, 11 aa, synthetic construct
ARQELVGAWFD

SEQ ID NO: 289 - protein, 10 aa, synthetic construct
VGGYNYVSWY

SEQ ID NO: 290 - protein, 10 aa, synthetic construct
LIIYEVSNRP

SEQ ID NO: 291 - protein, 10 aa, synthetic construct
ISYTSSSISC

SEQ ID NO: 292 - protein, 13 aa, synthetic construct
AASGFTFSSYGMH

SEQ ID NO: 293 - protein, 10 aa, synthetic construct
VISYDGSTKY

SEQ ID NO: 294 - protein, 14 aa, synthetic construct
AKDGTAVLVDRLDY

SEQ ID NO: 295 - protein, 11 aa, synthetic construct
RASQGISSYLA

SEQ ID NO: 296 - protein, 8 aa, synthetic construct
YAASTLQS

SEQ ID NO: 297 - protein, 9 aa, synthetic construct
QQLNSYPLT

SEQ ID NO: 298 - protein, 5 aa, synthetic construct
SYGMH

SEQ ID NO: 299 - protein, 17 aa, synthetic construct
VISYDGSTKYYADSVKG

SEQ ID NO: 300 - protein, 12 aa, synthetic construct
DGTAVLVDRLDY

SEQ ID NO: 301 - protein, 7 aa, synthetic construct
AASTLQS

SEQ ID NO: 302 - protein, 8 aa, synthetic construct
GFTFSSYG

SEQ ID NO: 303 - protein, 8 aa, synthetic construct
ISYDGSTK

SEQ ID NO: 304 - protein, 6 aa, synthetic construct
QGISSY

SEQ ID NO: 305 - protein, 7 aa, synthetic construct
GFTFSSY

SEQ ID NO: 306 - protein, 6 aa, synthetic construct
SYDGST

SEQ ID NO: 307 - protein, 6 aa, synthetic construct
SSYGMH

SEQ ID NO: 308 - protein, 13 aa, synthetic construct
WVAVISYDGSTKY

SEQ ID NO: 309 - protein, 13 aa, synthetic construct
AKDGTAVLVDRLD

SEQ ID NO: 310 - protein, 7 aa, synthetic construct
SSYLAWY

SEQ ID NO: 311 - protein, 10 aa, synthetic construct
LLIYAASTLQ

SEQ ID NO: 312 - protein, 8 aa, synthetic construct
QQLNSYPL

SEQ ID NO: 313 - protein, 13 aa, synthetic construct
AASGYDFDTYWIA

SEQ ID NO: 314 - protein, 10 aa, synthetic construct
VIYPDDSDTK

SEQ ID NO: 315 - protein, 7 aa, synthetic construct
ARATGDY

SEQ ID NO: 316 - protein, 11 aa, synthetic construct
RASQNVSTWLT

SEQ ID NO: 317 - protein, 8 aa, synthetic construct
YEASTLQS

SEQ ID NO: 318 - protein, 9 aa, synthetic construct
QQYRSYSRT

SEQ ID NO: 319 - protein, 5 aa, synthetic construct
TYWIA

SEQ ID NO: 320 - protein, 17 aa, synthetic construct
VIYPDDSDTKYSPSFQG

SEQ ID NO: 321 - protein, 5 aa, synthetic construct
ATGDY

SEQ ID NO: 322 - protein, 7 aa, synthetic construct
EASTLQS

SEQ ID NO: 323 - protein, 8 aa, synthetic construct
GYDFDTYW

SEQ ID NO: 324 - protein, 8 aa, synthetic construct
IYPDDSDT

SEQ ID NO: 325 - protein, 6 aa, synthetic construct
QNVSTW

SEQ ID NO: 326 - protein, 7 aa, synthetic construct
GYDFDTY

SEQ ID NO: 327 - protein, 6 aa, synthetic construct
YPDDSD

SEQ ID NO: 328 - protein, 13 aa, synthetic construct
AGSGYDFNTYWIA

SEQ ID NO: 329 - protein, 11 aa, synthetic construct
RASQNVSRWLT

SEQ ID NO: 330 - protein, 8 aa, synthetic construct
GYDFNTYW

SEQ ID NO: 331 - protein, 6 aa, synthetic construct
QNVSRW

SEQ ID NO: 332 - protein, 7 aa, synthetic construct
GYDFNTY

SEQ ID NO: 333 - protein, 6 aa, synthetic construct
NTYWIA

SEQ ID NO: 334 - protein, 7 aa, synthetic construct
SRWLTWY

SEQ ID NO: 335 - protein, 8 aa, synthetic construct
QQYRSYSR

SEQ ID NO: 336 - protein, 13 aa, synthetic construct
KASGATFSSYAIS

SEQ ID NO: 337 - protein, 10 aa, synthetic construct
RVTPIIDTTN

SEQ ID NO: 338 - protein, 15 aa, synthetic construct
ATRGTYYDFWPSMDV

SEQ ID NO: 339 - protein, 11 aa, synthetic construct
RASQDIRNYLA

SEQ ID NO: 340 - protein, 8 aa, synthetic construct
YAASSLHS

SEQ ID NO: 341 - protein, 9 aa, synthetic construct
QQYNSYPLT

SEQ ID NO: 342 - protein, 5 aa, synthetic construct
SYAIS

SEQ ID NO: 343 - protein, 17 aa, synthetic construct
RVTPIIDTTNYAQKFQG

SEQ ID NO: 344 - protein, 13 aa, synthetic construct
RGTYYDFWPSMDV

SEQ ID NO: 345 - protein, 7 aa, synthetic construct
AASSLHS

SEQ ID NO: 346 - protein, 8 aa, synthetic construct
GATFSSYA

SEQ ID NO: 347 - protein, 8 aa, synthetic construct
VTPIIDTT

SEQ ID NO: 348 - protein, 6 aa, synthetic construct
QDIRNY

SEQ ID NO: 349 - protein, 7 aa, synthetic construct
GATFSSY

SEQ ID NO: 350 - protein, 6 aa, synthetic construct
TPIIDT

SEQ ID NO: 351 - protein, 6 aa, synthetic construct
SSYAIS

SEQ ID NO: 352 - protein, 13 aa, synthetic construct
WLGRVTPIIDTTN

SEQ ID NO: 353 - protein, 14 aa, synthetic construct
ATRGTYYDFWPSMD

SEQ ID NO: 354 - protein, 7 aa, synthetic construct
RNYLAWF

SEQ ID NO: 355 - protein, 10 aa, synthetic construct
SLIYAASSLH

SEQ ID NO: 356 - protein, 8 aa, synthetic construct
QQYNSYPL

SEQ ID NO: 357 - protein, 13 aa, synthetic construct
AVYGGSFSGYYWS

SEQ ID NO: 358 - protein, 9 aa, synthetic construct
EINHSGSTN

SEQ ID NO: 359 - protein, 13 aa, synthetic construct
ASMSGSYYGAFDY

SEQ ID NO: 360 - protein, 11 aa, synthetic construct
SGDALPKQYAY

SEQ ID NO: 361 - protein, 8 aa, synthetic construct
YKDSERPS

SEQ ID NO: 362 - protein, 11 aa, synthetic construct
QSADSSGTYYV

SEQ ID NO: 363 - protein, 5 aa, synthetic construct
GYYWS

SEQ ID NO: 364 - protein, 16 aa, synthetic construct
EINHSGSTNYNPSLKS

SEQ ID NO: 365 - protein, 11 aa, synthetic construct
MSGSYYGAFDY

SEQ ID NO: 366 - protein, 7 aa, synthetic construct
KDSERPS

SEQ ID NO: 367 - protein, 8 aa, synthetic construct
GGSFSGYY

SEQ ID NO: 368 - protein, 7 aa, synthetic construct
INHSGST

SEQ ID NO: 369 - protein, 6 aa, synthetic construct
ALPKQY

SEQ ID NO: 370 - protein, 7 aa, synthetic construct
GGSFSGY

SEQ ID NO: 371 - protein, 5 aa, synthetic construct
NHSGS

SEQ ID NO: 372 - protein, 6 aa, synthetic construct
SGYYWS

SEQ ID NO: 373 - protein, 12 aa, synthetic construct
WIGEINHSGSTN

SEQ ID NO: 374 - protein, 12 aa, synthetic construct
ASMSGSYYGAFD

SEQ ID NO: 375 - protein, 10 aa, synthetic construct
LVIYKDSERP

SEQ ID NO: 376 - protein, 10 aa, synthetic construct
QSADSSGTYY

SEQ ID NO: 377 - protein, 15 aa, synthetic construct
TVSGGSISSSSYYWG

SEQ ID NO: 378 - protein, 9 aa, synthetic construct
SIYYSGSTY

SEQ ID NO: 379 - protein, 10 aa, synthetic construct
ASIIAALFDY

SEQ ID NO: 380 - protein, 11 aa, synthetic construct
SGDVLAKKYAR

SEQ ID NO: 381 - protein, 9 aa, synthetic construct
YSAADNNWV

SEQ ID NO: 382 - protein, 16 aa, synthetic construct
SIYYSGSTYYNPSLKS

SEQ ID NO: 383 - protein, 8 aa, synthetic construct
IIAALFDY

SEQ ID NO: 384 - protein, 10 aa, synthetic construct
GGSISSSSYY

SEQ ID NO: 385 - protein, 7 aa, synthetic construct
IYYSGST

SEQ ID NO: 386 - protein, 6 aa, synthetic construct
VLAKKY

SEQ ID NO: 387 - protein, 9 aa, synthetic construct
GGSISSSSY

SEQ ID NO: 388 - protein, 5 aa, synthetic construct
YYSGS

SEQ ID NO: 389 - protein, 12 aa, synthetic construct
WIGSIYYSGSTY

SEQ ID NO: 390 - protein, 9 aa, synthetic construct
ASIIAALFD

SEQ ID NO: 391 - protein, 7 aa, synthetic construct
KKYARWF

SEQ ID NO: 392 - protein, 8 aa, synthetic construct
YSAADNNW

SEQ ID NO: 393 - protein, 13 aa, synthetic construct
AASGFTFKHYSMH

SEQ ID NO: 394 - protein, 10 aa, synthetic construct
HISYDGSKTN

SEQ ID NO: 395 - protein, 14 aa, synthetic construct
VRDRTTGHDFGFDY

SEQ ID NO: 396 - protein, 11 aa, synthetic construct
SGDALSKKYAY

SEQ ID NO: 397 - protein, 8 aa, synthetic construct
FKDNERPA

SEQ ID NO: 398 - protein, 11 aa, synthetic construct
QSTDSDTSYVI

SEQ ID NO: 399 - protein, 5 aa, synthetic construct
HYSMH

SEQ ID NO: 400 - protein, 17 aa, synthetic construct
HISYDGSKTNYADSVKG

SEQ ID NO: 401 - protein, 12 aa, synthetic construct
DRTTGHDFGFDY

SEQ ID NO: 402 - protein, 7 aa, synthetic construct
KDNERPA

SEQ ID NO: 403 - protein, 8 aa, synthetic construct
GFTFKHYS

SEQ ID NO: 404 - protein, 8 aa, synthetic construct
ISYDGSKT

SEQ ID NO: 405 - protein, 6 aa, synthetic construct
ALSKKY

SEQ ID NO: 406 - protein, 7 aa, synthetic construct
GFTFKHY

SEQ ID NO: 407 - protein, 6 aa, synthetic construct
SYDGSK

SEQ ID NO: 408 - protein, 6 aa, synthetic construct
KHYSMH

SEQ ID NO: 409 - protein, 13 aa, synthetic construct
WVAHISYDGSKTN

SEQ ID NO: 410 - protein, 13 aa, synthetic construct
VRDRTTGHDFGFD

SEQ ID NO: 411 - protein, 7 aa, synthetic construct
KKYAYWY

SEQ ID NO: 412 - protein, 10 aa, synthetic construct
LVMFKDNERP

SEQ ID NO: 413 - protein, 10 aa, synthetic construct
QSTDSDTSYV

SEQ ID NO: 414 - protein, 13 aa, synthetic construct
KTSGGFFSSYRIN

SEQ ID NO: 415 - protein, 10 aa, synthetic construct
GLVPIFETAK

SEQ ID NO: 416 - protein, 13 aa, synthetic construct
ARGSGSGLTYFQT

SEQ ID NO: 417 - protein, 12 aa, synthetic construct
RASQSITTYYLA

SEQ ID NO: 418 - protein, 8 aa, synthetic construct
YGASSRAT

SEQ ID NO: 419 - protein, 8 aa, synthetic construct
QQYDSSPT

SEQ ID NO: 420 - protein, 5 aa, synthetic construct
SYRIN

SEQ ID NO: 421 - protein, 17 aa, synthetic construct
GLVPIFETAKYAQKFQG

SEQ ID NO: 422 - protein, 11 aa, synthetic construct
GSGSGLTYFQT

SEQ ID NO: 423 - protein, 7 aa, synthetic construct
GASSRAT

SEQ ID NO: 424 - protein, 8 aa, synthetic construct
GGFFSSYR

SEQ ID NO: 425 - protein, 8 aa, synthetic construct
LVPIFETA

SEQ ID NO: 426 - protein, 7 aa, synthetic construct
QSITTYY

SEQ ID NO: 427 - protein, 7 aa, synthetic construct
GGFFSSY

SEQ ID NO: 428 - protein, 6 aa, synthetic construct
VPIFET

SEQ ID NO: 429 - protein, 6 aa, synthetic construct
SSYRIN

SEQ ID NO: 430 - protein, 13 aa, synthetic construct
WMGGLVPIFETAK

SEQ ID NO: 431 - protein, 12 aa, synthetic construct
ARGSGSGLTYFQ

SEQ ID NO: 432 - protein, 8 aa, synthetic construct
TTYYLAWY

SEQ ID NO: 433 - protein, 10 aa, synthetic construct
LLIYGASSRA

SEQ ID NO: 434 - protein, 7 aa, synthetic construct
QQYDSSP

SEQ ID NO: 435 - protein, 15 aa, synthetic construct
TVSGGSITSISHYWG

SEQ ID NO: 436 - protein, 9 aa, synthetic construct
SMYYSGTTY

SEQ ID NO: 437 - protein, 10 aa, synthetic construct
ARHDVSRFDP

SEQ ID NO: 438 - protein, 14 aa, synthetic construct
TGTSSDVGGYNYVS

SEQ ID NO: 439 - protein, 8 aa, synthetic construct
YDVTNRPS

SEQ ID NO: 440 - protein, 9 aa, synthetic construct
SSYTTSTWV

SEQ ID NO: 441 - protein, 7 aa, synthetic construct
SISHYWG

SEQ ID NO: 442 - protein, 16 aa, synthetic construct
SMYYSGTTYYNPSLKS

SEQ ID NO: 443 - protein, 8 aa, synthetic construct
HDVSRFDP

SEQ ID NO: 444 - protein, 7 aa, synthetic construct
DVTNRPS

SEQ ID NO: 445 - protein, 10 aa, synthetic construct
GGSITSISHY

SEQ ID NO: 446 - protein, 7 aa, synthetic construct
MYYSGTT

SEQ ID NO: 447 - protein, 9 aa, synthetic construct
SSDVGGYNY

SEQ ID NO: 448 - protein, 9 aa, synthetic construct
GGSITSISH

SEQ ID NO: 449 - protein, 5 aa, synthetic construct
YYSGT

SEQ ID NO: 450 - protein, 8 aa, synthetic construct
TSISHYWG

SEQ ID NO: 451 - protein, 12 aa, synthetic construct
WIGSMYYSGTTY

SEQ ID NO: 452 - protein, 9 aa, synthetic construct
ARHDVSRFD

SEQ ID NO: 453 - protein, 10 aa, synthetic construct
LMIYDVTNRP

SEQ ID NO: 454 - protein, 8 aa, synthetic construct
SSYTTSTW

SEQ ID NO: 455 - protein, 13 aa, synthetic construct
VASGFTFSNFDMH

SEQ ID NO: 456 - protein, 10 aa, synthetic construct
VISYDESKRQ

SEQ ID NO: 457 - protein, 13 aa, synthetic construct
ATESTQYFGALDT

SEQ ID NO: 458 - protein, 11 aa, synthetic construct
SGDGLADQYVY

SEQ ID NO: 459 - protein, 10 aa, synthetic construct
QSTDRSGSYL

SEQ ID NO: 460 - protein, 5 aa, synthetic construct
NFDMH

SEQ ID NO: 461 - protein, 17 aa, synthetic construct
VISYDESKRQHADSVKG

SEQ ID NO: 462 - protein, 11 aa, synthetic construct
ESTQYFGALDT

SEQ ID NO: 463 - protein, 8 aa, synthetic construct
GFTFSNFD

SEQ ID NO: 464 - protein, 8 aa, synthetic construct
ISYDESKR

SEQ ID NO: 465 - protein, 6 aa, synthetic construct
GLADQY

SEQ ID NO: 466 - protein, 7 aa, synthetic construct
GFTFSNF

SEQ ID NO: 467 - protein, 6 aa, synthetic construct
SYDESK

SEQ ID NO: 468 - protein, 6 aa, synthetic construct
SNFDMH

SEQ ID NO: 469 - protein, 13 aa, synthetic construct
WVAVISYDESKRQ

SEQ ID NO: 470 - protein, 12 aa, synthetic construct
ATESTQYFGALD

SEQ ID NO: 471 - protein, 7 aa, synthetic construct
DQYVYWY

SEQ ID NO: 472 - protein, 10 aa, synthetic construct
LVMYKDSERP

SEQ ID NO: 473 - protein, 9 aa, synthetic construct
QSTDRSGSY

SEQ ID NO: 474 - protein, 13 aa, synthetic construct
AASGFNFNFYTMH

SEQ ID NO: 475 - protein, 10 aa, synthetic construct
LVFYDGSKTY

SEQ ID NO: 476 - protein, 14 aa, synthetic construct
VRDRTTGHDYGMDV

SEQ ID NO: 477 - protein, 11 aa, synthetic construct
SGAGLANLYTY

SEQ ID NO: 478 - protein, 11 aa, synthetic construct
QSADSSRSSVI

SEQ ID NO: 479 - protein, 5 aa, synthetic construct
FYTMH

SEQ ID NO: 480 - protein, 17 aa, synthetic construct
LVFYDGSKTYYADSVKG

SEQ ID NO: 481 - protein, 12 aa, synthetic construct
DRTTGHDYGMDV

SEQ ID NO: 482 - protein, 8 aa, synthetic construct
GFNFNFYT

SEQ ID NO: 483 - protein, 8 aa, synthetic construct
VFYDGSKT

SEQ ID NO: 484 - protein, 6 aa, synthetic construct
GLANLY

SEQ ID NO: 485 - protein, 7 aa, synthetic construct
GFNFNFY

SEQ ID NO: 486 - protein, 6 aa, synthetic construct
FYDGSK

SEQ ID NO: 487 - protein, 6 aa, synthetic construct
NFYTMH

SEQ ID NO: 488 - protein, 13 aa, synthetic construct
WVALVFYDGSKTY

SEQ ID NO: 489 - protein, 13 aa, synthetic construct
VRDRTTGHDYGMD

SEQ ID NO: 490 - protein, 7 aa, synthetic construct
NLYTYWY

SEQ ID NO: 491 - protein, 10 aa, synthetic construct
LLIYKDTERP

SEQ ID NO: 492 - protein, 10 aa, synthetic construct
QSADSSRSSV

SEQ ID NO: 493 - protein, 15 aa, synthetic construct
TFSGFSLSTGGVCIN

SEQ ID NO: 494 - protein, 9 aa, synthetic construct
RIDWDNDKY

SEQ ID NO: 495 - protein, 16 aa, synthetic construct
ARMTGLYSDNWKGFDI

SEQ ID NO: 496 - protein, 12 aa, synthetic construct
RASQSVDSTYLA

SEQ ID NO: 497 - protein, 8 aa, synthetic construct
YSASRRAT

SEQ ID NO: 498 - protein, 10 aa, synthetic construct
QQYGSAPGYT

SEQ ID NO: 499 - protein, 7 aa, synthetic construct
TGGVCIN

SEQ ID NO: 500 - protein, 16 aa, synthetic construct
RIDWDNDKYYRPSLKT

SEQ ID NO: 501 - protein, 14 aa, synthetic construct
MTGLYSDNWKGFDI

SEQ ID NO: 502 - protein, 7 aa, synthetic construct
SASRRAT

SEQ ID NO: 503 - protein, 10 aa, synthetic construct
GFSLSTGGVC

SEQ ID NO: 504 - protein, 7 aa, synthetic construct
IDWDNDK

SEQ ID NO: 505 - protein, 7 aa, synthetic construct
QSVDSTY

SEQ ID NO: 506 - protein, 9 aa, synthetic construct
GFSLSTGGV

SEQ ID NO: 507 - protein, 5 aa, synthetic construct
DWDND

SEQ ID NO: 508 - protein, 8 aa, synthetic construct
STGGVCIN

SEQ ID NO: 509 - protein, 12 aa, synthetic construct
WLARIDWDNDKY

SEQ ID NO: 510 - protein, 15 aa, synthetic construct
ARMTGLYSDNWKGFD

SEQ ID NO: 511 - protein, 8 aa, synthetic construct
DSTYLAWY

SEQ ID NO: 512 - protein, 10 aa, synthetic construct
LLIYSASRRA

SEQ ID NO: 513 - protein, 9 aa, synthetic construct
QQYGSAPGY

SEQ ID NO: 514 - protein, 13 aa, synthetic construct
KVSGYRFTSYWIG

SEQ ID NO: 515 - protein, 10 aa, synthetic construct
VIYPPDSDTA

SEQ ID NO: 516 - protein, 9 aa, synthetic construct
ARQGRGFDY

SEQ ID NO: 517 - protein, 11 aa, synthetic construct
RTSQNIYTYLN

SEQ ID NO: 518 - protein, 8 aa, synthetic construct
NTASSLQG

SEQ ID NO: 519 - protein, 9 aa, synthetic construct
QQSYDSPRT

SEQ ID NO: 520 - protein, 5 aa, synthetic construct
SYWIG

SEQ ID NO: 521 - protein, 17 aa, synthetic construct
VIYPPDSDTAYSPSFQG

SEQ ID NO: 522 - protein, 7 aa, synthetic construct
QGRGFDY

SEQ ID NO: 523 - protein, 7 aa, synthetic construct
TASSLQG

SEQ ID NO: 524 - protein, 8 aa, synthetic construct
GYRFTSYW

SEQ ID NO: 525 - protein, 8 aa, synthetic construct
IYPPDSDT

SEQ ID NO: 526 - protein, 6 aa, synthetic construct
QNIYTY

SEQ ID NO: 527 - protein, 7 aa, synthetic construct
GYRFTSY

SEQ ID NO: 528 - protein, 6 aa, synthetic construct
YPPDSD

SEQ ID NO: 529 - protein, 6 aa, synthetic construct
TSYWIG

SEQ ID NO: 530 - protein, 13 aa, synthetic construct
WMGVIYPPDSDTA

SEQ ID NO: 531 - protein, 8 aa, synthetic construct
ARQGRGFD

SEQ ID NO: 532 - protein, 7 aa, synthetic construct
YTYLNWY

SEQ ID NO: 533 - protein, 10 aa, synthetic construct
LLINTASSLQ

SEQ ID NO: 534 - protein, 8 aa, synthetic construct
QQSYDSPR

SEQ ID NO: 535 - protein, 15 aa, synthetic construct
TVSGFSLNTAEVCWN

SEQ ID NO: 536 - protein, 16 aa, synthetic construct
ARMTGLYTDNWKGFDM

SEQ ID NO: 537 - protein, 12 aa, synthetic construct
RASQTIDSTYFA

SEQ ID NO: 538 - protein, 10 aa, synthetic construct
QQYGHAPGYT

SEQ ID NO: 539 - protein, 7 aa, synthetic construct
TAEVCWN

SEQ ID NO: 540 - protein, 16 aa, synthetic construct
RIDWDDDKYIRPSLKT

SEQ ID NO: 541 - protein, 14 aa, synthetic construct
MTGLYTDNWKGFDM

SEQ ID NO: 542 - protein, 10 aa, synthetic construct
GFSLNTAEVC

SEQ ID NO: 543 - protein, 9 aa, synthetic construct
GFSLNTAEV

SEQ ID NO: 544 - protein, 8 aa, synthetic construct
NTAEVCWN

SEQ ID NO: 545 - protein, 15 aa, synthetic construct
ARMTGLYTDNWKGFD

SEQ ID NO: 546 - protein, 8 aa, synthetic construct
DSTYFAWY

SEQ ID NO: 547 - protein, 9 aa, synthetic construct
QQYGHAPGY

SEQ ID NO: 548 - protein, 13 aa, synthetic construct
KAPGGTFSRYAIS

SEQ ID NO: 549 - protein, 10 aa, synthetic construct
GIIPMFDITN

SEQ ID NO: 550 - protein, 19 aa, synthetic construct
ATESCSGGNCYWDHDAFDI

SEQ ID NO: 551 - protein, 12 aa, synthetic construct
RASQSLSSSYLA

SEQ ID NO: 552 - protein, 8 aa, synthetic construct
YGASTRAT

SEQ ID NO: 553 - protein, 5 aa, synthetic construct
RYAIS

SEQ ID NO: 554 - protein, 17 aa, synthetic construct
GIIPMFDITNYAQKLQD

SEQ ID NO: 555 - protein, 17 aa, synthetic construct
ESCSGGNCYWDHDAFDI

SEQ ID NO: 556 - protein, 7 aa, synthetic construct
GASTRAT

SEQ ID NO: 557 - protein, 8 aa, synthetic construct
GGTFSRYA

SEQ ID NO: 558 - protein, 8 aa, synthetic construct
IIPMFDIT

SEQ ID NO: 559 - protein, 7 aa, synthetic construct
QSLSSSY

SEQ ID NO: 560 - protein, 7 aa, synthetic construct
GGTFSRY

SEQ ID NO: 561 - protein, 6 aa, synthetic construct
IPMFDI

SEQ ID NO: 562 - protein, 6 aa, synthetic construct
SRYAIS

SEQ ID NO: 563 - protein, 13 aa, synthetic construct
WMGGIIPMFDITN

SEQ ID NO: 564 - protein, 18 aa, synthetic construct
ATESCSGGNCYWDHDAFD

SEQ ID NO: 565 - protein, 8 aa, synthetic construct
SSSYLAWY

SEQ ID NO: 566 - protein, 10 aa, synthetic construct
LLIYGASTRA

SEQ ID NO: 567 - protein, 9 aa, synthetic construct
QQYGRSLPR

SEQ ID NO: 568 - protein, 13 aa, synthetic construct
KASGGTFSSYAIS

SEQ ID NO: 569 - protein, 10 aa, synthetic construct
GIIPIFDIAN

SEQ ID NO: 570 - protein, 19 aa, synthetic construct
ASENCGGGNCYWEHDAFDI

SEQ ID NO: 571 - protein, 12 aa, synthetic construct
RASQSVSGGDLA

SEQ ID NO: 572 - protein, 8 aa, synthetic construct
YGASIRAT

SEQ ID NO: 573 - protein, 10 aa, synthetic construct
QHYGNSPPRT

SEQ ID NO: 574 - protein, 17 aa, synthetic construct
GIIPIFDIANYAQKFQG

SEQ ID NO: 575 - protein, 7 aa, synthetic construct
GASIRAT

SEQ ID NO: 576 - protein, 8 aa, synthetic construct
GGTFSSYA

SEQ ID NO: 577 - protein, 8 aa, synthetic construct
IIPIFDIA

SEQ ID NO: 578 - protein, 7 aa, synthetic construct
QSVSGGD

SEQ ID NO: 579 - protein, 7 aa, synthetic construct
GGTFSSY

SEQ ID NO: 580 - protein, 6 aa, synthetic construct
IPIFDI

SEQ ID NO: 581 - protein, 13 aa, synthetic construct
WMGGIIPIFDIAN

SEQ ID NO: 582 - protein, 18 aa, synthetic construct
ASENCGGGNCYWEHDAFD

SEQ ID NO: 583 - protein, 8 aa, synthetic construct
SGGDLAWY

SEQ ID NO: 584 - protein, 10 aa, synthetic construct
LLMYGASIRA

SEQ ID NO: 585 - protein, 9 aa, synthetic construct
QHYGNSPPR

SEQ ID NO: 586 - protein, 13 aa, synthetic construct
KAYGGTFSTYAIS

SEQ ID NO: 587 - protein, 19 aa, synthetic construct
ASENCSGGNCYWEHDAFDI

SEQ ID NO: 588 - protein, 10 aa, synthetic construct
QQYGNSPPRT

SEQ ID NO: 589 - protein, 5 aa, synthetic construct
TYAIS

SEQ ID NO: 590 - protein, 17 aa, synthetic construct
ENCSGGNCYWEHDAFDI

SEQ ID NO: 591 - protein, 8 aa, synthetic construct
GGTFSTYA

SEQ ID NO: 592 - protein, 7 aa, synthetic construct
GGTFSTY

SEQ ID NO: 593 - protein, 6 aa, synthetic construct
STYAIS

SEQ ID NO: 594 - protein, 18 aa, synthetic construct
ASENCSGGNCYWEHDAFD

SEQ ID NO: 595 - protein, 9 aa, synthetic construct
QQYGNSPPR

SEQ ID NO: 596 - protein, 13 aa, synthetic construct
QTSGYSFASFWIG

SEQ ID NO: 597 - protein, 10 aa, synthetic construct
IIYPSDSDTT

SEQ ID NO: 598 - protein, 11 aa, synthetic construct
AKHGVGAAIDH

SEQ ID NO: 599 - protein, 11 aa, synthetic construct
RTSQVISGYLN

SEQ ID NO: 600 - protein, 8 aa, synthetic construct
YTTSTLQP

SEQ ID NO: 601 - protein, 9 aa, synthetic construct
QQSYSSPRT

SEQ ID NO: 602 - protein, 5 aa, synthetic construct
SFWIG

SEQ ID NO: 603 - protein, 17 aa, synthetic construct
IIYPSDSDTTYSPTFQG

SEQ ID NO: 604 - protein, 9 aa, synthetic construct
HGVGAAIDH

SEQ ID NO: 605 - protein, 7 aa, synthetic construct
TTSTLQP

SEQ ID NO: 606 - protein, 8 aa, synthetic construct
GYSFASFW

SEQ ID NO: 607 - protein, 8 aa, synthetic construct
IYPSDSDT

SEQ ID NO: 608 - protein, 6 aa, synthetic construct
QVISGY

SEQ ID NO: 609 - protein, 7 aa, synthetic construct
GYSFASF

SEQ ID NO: 610 - protein, 6 aa, synthetic construct
YPSDSD

SEQ ID NO: 611 - protein, 6 aa, synthetic construct
ASFWIG

SEQ ID NO: 612 - protein, 13 aa, synthetic construct
WMGIIYPSDSDTT

SEQ ID NO: 613 - protein, 8 aa, synthetic construct
HGVGAAID

SEQ ID NO: 614 - protein, 7 aa, synthetic construct
SGYLNWY

SEQ ID NO: 615 - protein, 10 aa, synthetic construct
LLIYTTSTLQ

SEQ ID NO: 616 - protein, 8 aa, synthetic construct
QQSYSSPR

SEQ ID NO: 617 - protein, 15 aa, synthetic construct
TVSGGSIGGGPYHWS

SEQ ID NO: 618 - protein, 9 aa, synthetic construct
HVETSGDTK

SEQ ID NO: 619 - protein, 11 aa, synthetic construct
ARGTAMDAFDI

SEQ ID NO: 620 - protein, 11 aa, synthetic construct
RASQDISNWLA

SEQ ID NO: 621 - protein, 8 aa, synthetic construct
YVASGLQS

SEQ ID NO: 622 - protein, 9 aa, synthetic construct
QQGNSFPLT

SEQ ID NO: 623 - protein, 7 aa, synthetic construct
GGPYHWS

SEQ ID NO: 624 - protein, 16 aa, synthetic construct
HVETSGDTKTNPSFRS

SEQ ID NO: 625 - protein, 9 aa, synthetic construct
GTAMDAFDI

SEQ ID NO: 626 - protein, 7 aa, synthetic construct
VASGLQS

SEQ ID NO: 627 - protein, 10 aa, synthetic construct
GGSIGGGPYH

SEQ ID NO: 628 - protein, 7 aa, synthetic construct
VETSGDT

SEQ ID NO: 629 - protein, 6 aa, synthetic construct
QDISNW

SEQ ID NO: 630 - protein, 9 aa, synthetic construct
GGSIGGGPY

SEQ ID NO: 631 - protein, 5 aa, synthetic construct
ETSGD

SEQ ID NO: 632 - protein, 8 aa, synthetic construct
GGGPYHWS

SEQ ID NO: 633 - protein, 12 aa, synthetic construct
WIGHVETSGDTK

SEQ ID NO: 634 - protein, 8 aa, synthetic construct
GTAMDAFD

SEQ ID NO: 635 - protein, 7 aa, synthetic construct
SNWLAWY

SEQ ID NO: 636 - protein, 10 aa, synthetic construct
LLIYVASGLQ

SEQ ID NO: 637 - protein, 8 aa, synthetic construct
QQGNSFPL

SEQ ID NO: 638 - protein, 13 aa, synthetic construct
AASGFSFSGYGMH

SEQ ID NO: 639 - protein, 10 aa, synthetic construct
FIWYDGSSKK

SEQ ID NO: 640 - protein, 10 aa, synthetic construct
ARDVLGELPD

SEQ ID NO: 641 - protein, 11 aa, synthetic construct
RASQGISNSLG

SEQ ID NO: 642 - protein, 8 aa, synthetic construct
YAASSLQS

SEQ ID NO: 643 - protein, 9 aa, synthetic construct
QQYNTYPLT

SEQ ID NO: 644 - protein, 5 aa, synthetic construct
GYGMH

SEQ ID NO: 645 - protein, 17 aa, synthetic construct
FIWYDGSSKKYGDSVKG

SEQ ID NO: 646 - protein, 8 aa, synthetic construct
DVLGELPD

SEQ ID NO: 647 - protein, 7 aa, synthetic construct
AASSLQS

SEQ ID NO: 648 - protein, 8 aa, synthetic construct
GFSFSGYG

SEQ ID NO: 649 - protein, 8 aa, synthetic construct
IWYDGSSK

SEQ ID NO: 650 - protein, 6 aa, synthetic construct
QGISNS

SEQ ID NO: 651 - protein, 7 aa, synthetic construct
GFSFSGY

SEQ ID NO: 652 - protein, 6 aa, synthetic construct
WYDGSS

SEQ ID NO: 653 - protein, 6 aa, synthetic construct
SGYGMH

SEQ ID NO: 654 - protein, 13 aa, synthetic construct
WVAFIWYDGSSKK

SEQ ID NO: 655 - protein, 9 aa, synthetic construct
ARDVLGELP

SEQ ID NO: 656 - protein, 7 aa, synthetic construct
SNSLGWF

SEQ ID NO: 657 - protein, 10 aa, synthetic construct
SLIYAASSLQ

SEQ ID NO: 658 - protein, 8 aa, synthetic construct
QQYNTYPL

SEQ ID NO: 659 - protein, 13 aa, synthetic construct
ATSGFTFSNYGLH

SEQ ID NO: 660 - protein, 9 aa, synthetic construct
AIWFDGRTF

SEQ ID NO: 661 - protein, 14 aa, synthetic construct
ARETWSYAGGVFDY

SEQ ID NO: 662 - protein, 12 aa, synthetic construct
TLSSGHSGHAIA

SEQ ID NO: 663 - protein, 12 aa, synthetic construct
KVHSDGSHNKGD

SEQ ID NO: 664 - protein, 9 aa, synthetic construct
QTWDTGLWV

SEQ ID NO: 665 - protein, 5 aa, synthetic construct
NYGLH

SEQ ID NO: 666 - protein, 16 aa, synthetic construct
AIWFDGRTFYADSVKD

SEQ ID NO: 667 - protein, 12 aa, synthetic construct
ETWSYAGGVFDY

SEQ ID NO: 668 - protein, 11 aa, synthetic construct
VHSDGSHNKGD

SEQ ID NO: 669 - protein, 8 aa, synthetic construct
GFTFSNYG

SEQ ID NO: 670 - protein, 7 aa, synthetic construct
IWFDGRT

SEQ ID NO: 671 - protein, 7 aa, synthetic construct
SGHSGHA

SEQ ID NO: 672 - protein, 6 aa, synthetic construct
VHSDGS

SEQ ID NO: 673 - protein, 7 aa, synthetic construct
GFTFSNY

SEQ ID NO: 674 - protein, 5 aa, synthetic construct
WFDGR

SEQ ID NO: 675 - protein, 6 aa, synthetic construct
SNYGLH

SEQ ID NO: 676 - protein, 12 aa, synthetic construct
WLAAIWFDGRTF

SEQ ID NO: 677 - protein, 13 aa, synthetic construct
ARETWSYAGGVFD

SEQ ID NO: 678 - protein, 8 aa, synthetic construct
SGHAIAWH

SEQ ID NO: 679 - protein, 14 aa, synthetic construct
FLMKVHSDGSHNKG

SEQ ID NO: 680 - protein, 8 aa, synthetic construct
QTWDTGLW

SEQ ID NO: 681 - protein, 13 aa, synthetic construct
KASGYTFTTYYIH

SEQ ID NO: 682 - protein, 10 aa, synthetic construct
VLNPSRNRTI

SEQ ID NO: 683 - protein, 15 aa, synthetic construct
ARDAGLGPYYYYMDV

SEQ ID NO: 684 - protein, 11 aa, synthetic construct
SGDGLTKQYAF

SEQ ID NO: 685 - protein, 11 aa, synthetic construct
QSADRTDIYVV

SEQ ID NO: 686 - protein, 5 aa, synthetic construct
TYYIH

SEQ ID NO: 687 - protein, 17 aa, synthetic construct
VLNPSRNRTIYAQKFQG

SEQ ID NO: 688 - protein, 13 aa, synthetic construct
DAGLGPYYYYMDV

SEQ ID NO: 689 - protein, 8 aa, synthetic construct
GYTFTTYY

SEQ ID NO: 690 - protein, 8 aa, synthetic construct
LNPSRNRT

SEQ ID NO: 691 - protein, 6 aa, synthetic construct
GLTKQY

SEQ ID NO: 692 - protein, 7 aa, synthetic construct
GYTFTTY

SEQ ID NO: 693 - protein, 6 aa, synthetic construct
NPSRNR

SEQ ID NO: 694 - protein, 6 aa, synthetic construct
TTYYIH

SEQ ID NO: 695 - protein, 13 aa, synthetic construct
WVGVLNPSRNRTI

SEQ ID NO: 696 - protein, 14 aa, synthetic construct
ARDAGLGPYYYYMD

SEQ ID NO: 697 - protein, 7 aa, synthetic construct
KQYAFWY

SEQ ID NO: 698 - protein, 10 aa, synthetic construct
QSADRTDIYV

SEQ ID NO: 699 - protein, 13 aa, synthetic construct
KTSGGTFSSATLS

SEQ ID NO: 700 - protein, 10 aa, synthetic construct
GITPMFTTAN

SEQ ID NO: 701 - protein, 15 aa, synthetic construct
ARQFGHESRDSYYLY

SEQ ID NO: 702 - protein, 12 aa, synthetic construct
RASQTLSSSYLA

SEQ ID NO: 703 - protein, 8 aa, synthetic construct
YGASSRVA

SEQ ID NO: 704 - protein, 8 aa, synthetic construct
QQYGSSRT

SEQ ID NO: 705 - protein, 5 aa, synthetic construct
SATLS

SEQ ID NO: 706 - protein, 17 aa, synthetic construct
GITPMFTTANYAQKFQG

SEQ ID NO: 707 - protein, 13 aa, synthetic construct
QFGHESRDSYYLY

SEQ ID NO: 708 - protein, 7 aa, synthetic construct
GASSRVA

SEQ ID NO: 709 - protein, 8 aa, synthetic construct
GGTFSSAT

SEQ ID NO: 710 - protein, 8 aa, synthetic construct
ITPMFTTA

SEQ ID NO: 711 - protein, 7 aa, synthetic construct
QTLSSSY

SEQ ID NO: 712 - protein, 7 aa, synthetic construct
GGTFSSA

SEQ ID NO: 713 - protein, 6 aa, synthetic construct
TPMFTT

SEQ ID NO: 714 - protein, 6 aa, synthetic construct
SSATLS

SEQ ID NO: 715 - protein, 13 aa, synthetic construct
WMGGITPMFTTAN

SEQ ID NO: 716 - protein, 14 aa, synthetic construct
ARQFGHESRDSYYL

SEQ ID NO: 717 - protein, 10 aa, synthetic construct
LLIYGASSRV

SEQ ID NO: 718 - protein, 7 aa, synthetic construct
QQYGSSR

SEQ ID NO: 719 - protein, 14 aa, synthetic construct
TVSGYSISSGYYWG

SEQ ID NO: 720 - protein, 9 aa, synthetic construct
SIYHSGSTY

SEQ ID NO: 721 - protein, 14 aa, synthetic construct
ARSARAVVMDAFHI

SEQ ID NO: 722 - protein, 13 aa, synthetic construct
TGSSGSIASNYVQ

SEQ ID NO: 723 - protein, 8 aa, synthetic construct
YEDNQRPS

SEQ ID NO: 724 - protein, 10 aa, synthetic construct
QSYDNIHPWV

SEQ ID NO: 725 - protein, 6 aa, synthetic construct
SGYYWG

SEQ ID NO: 726 - protein, 16 aa, synthetic construct
SIYHSGSTYYNPSLNS

SEQ ID NO: 727 - protein, 12 aa, synthetic construct
SARAVVMDAFHI

SEQ ID NO: 728 - protein, 7 aa, synthetic construct
EDNQRPS

SEQ ID NO: 729 - protein, 9 aa, synthetic construct
GYSISSGYY

SEQ ID NO: 730 - protein, 7 aa, synthetic construct
IYHSGST

SEQ ID NO: 731 - protein, 8 aa, synthetic construct
SGSIASNY

SEQ ID NO: 732 - protein, 8 aa, synthetic construct
GYSISSGY

SEQ ID NO: 733 - protein, 5 aa, synthetic construct
YHSGS

SEQ ID NO: 734 - protein, 7 aa, synthetic construct
SSGYYWG

SEQ ID NO: 735 - protein, 12 aa, synthetic construct
WIGSIYHSGSTY

SEQ ID NO: 736 - protein, 13 aa, synthetic construct
ARSARAVVMDAFH

SEQ ID NO: 737 - protein, 9 aa, synthetic construct
IASNYVQWY

SEQ ID NO: 738 - protein, 10 aa, synthetic construct
TVIYEDNQRP

SEQ ID NO: 739 - protein, 9 aa, synthetic construct
QSYDNIHPW

SEQ ID NO: 740 - protein, 11 aa, synthetic construct
GGNNIGSKSVH

SEQ ID NO: 741 - protein, 8 aa, synthetic construct
YYDSDRPS

SEQ ID NO: 742 - protein, 12 aa, synthetic construct
QVWDGSTDHFYV

SEQ ID NO: 743 - protein, 7 aa, synthetic construct
YDSDRPS

SEQ ID NO: 744 - protein, 6 aa, synthetic construct
NIGSKS

SEQ ID NO: 745 - protein, 7 aa, synthetic construct
SKSVHWY

SEQ ID NO: 746 - protein, 10 aa, synthetic construct
LVIYYDSDRP

SEQ ID NO: 747 - protein, 11 aa, synthetic construct
QVWDGSTDHFY

SEQ ID NO: 748 - protein, 15 aa, synthetic construct
TVSGGSISSGEYYWS

SEQ ID NO: 749 - protein, 9 aa, synthetic construct
YISYAGNTY

SEQ ID NO: 750 - protein, 17 aa, synthetic construct
ARDFVGTNWYDRYAFHI

SEQ ID NO: 751 - protein, 14 aa, synthetic construct
TGTSSDVGGYHYVS

SEQ ID NO: 752 - protein, 8 aa, synthetic construct
YDVTKRPS

SEQ ID NO: 753 - protein, 11 aa, synthetic construct
CSYAGSYAFWV

SEQ ID NO: 754 - protein, 7 aa, synthetic construct
SGEYYWS

SEQ ID NO: 755 - protein, 16 aa, synthetic construct
YISYAGNTYYNPSLKS

SEQ ID NO: 756 - protein, 15 aa, synthetic construct
DFVGTNWYDRYAFHI

SEQ ID NO: 757 - protein, 7 aa, synthetic construct
DVTKRPS

SEQ ID NO: 758 - protein, 10 aa, synthetic construct
GGSISSGEYY

SEQ ID NO: 759 - protein, 7 aa, synthetic construct
ISYAGNT

SEQ ID NO: 760 - protein, 9 aa, synthetic construct
SSDVGGYHY

SEQ ID NO: 761 - protein, 9 aa, synthetic construct
GGSISSGEY

SEQ ID NO: 762 - protein, 5 aa, synthetic construct
SYAGN

SEQ ID NO: 763 - protein, 8 aa, synthetic construct
SSGEYYWS

SEQ ID NO: 764 - protein, 12 aa, synthetic construct
WIGYISYAGNTY

SEQ ID NO: 765 - protein, 16 aa, synthetic construct
ARDFVGTNWYDRYAFH

SEQ ID NO: 766 - protein, 10 aa, synthetic construct
VGGYHYVSWY

SEQ ID NO: 767 - protein, 10 aa, synthetic construct
LILYDVTKRP

SEQ ID NO: 768 - protein, 10 aa, synthetic construct
CSYAGSYAFW

SEQ ID NO: 769 - protein, 13 aa, synthetic construct
GASGFNFIDYAMY

SEQ ID NO: 770 - protein, 10 aa, synthetic construct
SITNDAKSTF

SEQ ID NO: 771 - protein, 15 aa, synthetic construct
ARGRSHYYDSSGFDY

SEQ ID NO: 772 - protein, 11 aa, synthetic construct
GGDSLGSKSVH

SEQ ID NO: 773 - protein, 8 aa, synthetic construct
FDDSDRPS

SEQ ID NO: 774 - protein, 10 aa, synthetic construct
QVWDAGALWV

SEQ ID NO: 775 - protein, 5 aa, synthetic construct
DYAMY

SEQ ID NO: 776 - protein, 17 aa, synthetic construct
SITNDAKSTFYGNSVKG

SEQ ID NO: 777 - protein, 13 aa, synthetic construct
GRSHYYDSSGFDY

SEQ ID NO: 778 - protein, 7 aa, synthetic construct
DDSDRPS

SEQ ID NO: 779 - protein, 8 aa, synthetic construct
GFNFIDYA

SEQ ID NO: 780 - protein, 8 aa, synthetic construct
ITNDAKST

SEQ ID NO: 781 - protein, 6 aa, synthetic construct
SLGSKS

SEQ ID NO: 782 - protein, 7 aa, synthetic construct
GFNFIDY

SEQ ID NO: 783 - protein, 6 aa, synthetic construct
TNDAKS

SEQ ID NO: 784 - protein, 6 aa, synthetic construct
IDYAMY

SEQ ID NO: 785 - protein, 13 aa, synthetic construct
YVSSITNDAKSTF

SEQ ID NO: 786 - protein, 14 aa, synthetic construct
ARGRSHYYDSSGFD

SEQ ID NO: 787 - protein, 10 aa, synthetic construct
LVVFDDSDRP

SEQ ID NO: 788 - protein, 9 aa, synthetic construct
QVWDAGALW

SEQ ID NO: 789 - protein, 15 aa, synthetic construct
SVSGDSISTTSYYWD

SEQ ID NO: 790 - protein, 9 aa, synthetic construct
SINYSGNTY

SEQ ID NO: 791 - protein, 16 aa, synthetic construct
ARRGVPAGMVPSWIDP

SEQ ID NO: 792 - protein, 11 aa, synthetic construct
QASEDIRNYLN

SEQ ID NO: 793 - protein, 8 aa, synthetic construct
YDASNLKT

SEQ ID NO: 794 - protein, 9 aa, synthetic construct
QQYDTVPLT

SEQ ID NO: 795 - protein, 7 aa, synthetic construct
TTSYYWD

SEQ ID NO: 796 - protein, 16 aa, synthetic construct
SINYSGNTYYSPLLKS

SEQ ID NO: 797 - protein, 14 aa, synthetic construct
RGVPAGMVPSWIDP

SEQ ID NO: 798 - protein, 7 aa, synthetic construct
DASNLKT

SEQ ID NO: 799 - protein, 10 aa, synthetic construct
GDSISTTSYY

SEQ ID NO: 800 - protein, 7 aa, synthetic construct
INYSGNT

SEQ ID NO: 801 - protein, 6 aa, synthetic construct
EDIRNY

SEQ ID NO: 802 - protein, 9 aa, synthetic construct
GDSISTTSY

SEQ ID NO: 803 - protein, 5 aa, synthetic construct
NYSGN

SEQ ID NO: 804 - protein, 8 aa, synthetic construct
STTSYYWD

SEQ ID NO: 805 - protein, 12 aa, synthetic construct
WIGSINYSGNTY

SEQ ID NO: 806 - protein, 15 aa, synthetic construct
ARRGVPAGMVPSWID

SEQ ID NO: 807 - protein, 7 aa, synthetic construct
RNYLNWY

SEQ ID NO: 808 - protein, 10 aa, synthetic construct
VLIYDASNLK

SEQ ID NO: 809 - protein, 8 aa, synthetic construct
QQYDTVPL

SEQ ID NO: 810 - protein, 13 aa, synthetic construct
KGSGYHFPTYWIA

SEQ ID NO: 811 - protein, 10 aa, synthetic construct
VIYPRDSDTK

SEQ ID NO: 812 - protein, 11 aa, synthetic construct
RAGENVSTWLT

SEQ ID NO: 813 - protein, 8 aa, synthetic construct
YKASTLET

SEQ ID NO: 814 - protein, 9 aa, synthetic construct
QQYKSYSRT

SEQ ID NO: 815 - protein, 17 aa, synthetic construct
VIYPRDSDTKYSPSFQG

SEQ ID NO: 816 - protein, 7 aa, synthetic construct
KASTLET

SEQ ID NO: 817 - protein, 8 aa, synthetic construct
GYHFPTYW

SEQ ID NO: 818 - protein, 8 aa, synthetic construct
IYPRDSDT

SEQ ID NO: 819 - protein, 6 aa, synthetic construct
ENVSTW

SEQ ID NO: 820 - protein, 7 aa, synthetic construct
GYHFPTY

SEQ ID NO: 821 - protein, 6 aa, synthetic construct
YPRDSD

SEQ ID NO: 822 - protein, 6 aa, synthetic construct
PTYWIA

SEQ ID NO: 823 - protein, 13 aa, synthetic construct
WMGVIYPRDSDTK

SEQ ID NO: 824 - protein, 10 aa, synthetic construct
RLMYKASTLE

SEQ ID NO: 825 - protein, 8 aa, synthetic construct
QQYKSYSR

SEQ ID NO: 826 - protein, 13 aa, synthetic construct
QGFGYSFPTYWIA

SEQ ID NO: 827 - protein, 10 aa, synthetic construct
IIYPDDSDTT

SEQ ID NO: 828 - protein, 7 aa, synthetic construct
ARGMGDH

SEQ ID NO: 829 - protein, 16 aa, synthetic construct
KSSQSLLFGDGATYLY

SEQ ID NO: 830 - protein, 8 aa, synthetic construct
SKVSSRFS

SEQ ID NO: 831 - protein, 9 aa, synthetic construct
MQALHMPRT

SEQ ID NO: 832 - protein, 17 aa, synthetic construct
IIYPDDSDTTYSPSFQG

SEQ ID NO: 833 - protein, 5 aa, synthetic construct
GMGDH

SEQ ID NO: 834 - protein, 7 aa, synthetic construct
KVSSRFS

SEQ ID NO: 835 - protein, 8 aa, synthetic construct
GYSFPTYW

SEQ ID NO: 836 - protein, 11 aa, synthetic construct
QSLLFGDGATY

SEQ ID NO: 837 - protein, 7 aa, synthetic construct
GYSFPTY

SEQ ID NO: 838 - protein, 13 aa, synthetic construct
WMGIIYPDDSDTT

SEQ ID NO: 839 - protein, 6 aa, synthetic construct
ARGMGD

SEQ ID NO: 840 - protein, 12 aa, synthetic construct
LFGDGATYLYWF

SEQ ID NO: 841 - protein, 10 aa, synthetic construct
LLISKVSSRF

SEQ ID NO: 842 - protein, 8 aa, synthetic construct
MQALHMPR

SEQ ID NO: 843 - protein, 13 aa, synthetic construct
AASGFTVSSNYMS

SEQ ID NO: 844 - protein, 9 aa, synthetic construct
VIYSGGSTY

SEQ ID NO: 845 - protein, 15 aa, synthetic construct
ARDRKIVVRRGGLDI

SEQ ID NO: 846 - protein, 9 aa, synthetic construct
QQYYSYPHT

SEQ ID NO: 847 - protein, 5 aa, synthetic construct
SNYMS

SEQ ID NO: 848 - protein, 16 aa, synthetic construct
VIYSGGSTYYADSVKG

SEQ ID NO: 849 - protein, 13 aa, synthetic construct
DRKIVVRRGGLDI

SEQ ID NO: 850 - protein, 8 aa, synthetic construct
GFTVSSNY

SEQ ID NO: 851 - protein, 7 aa, synthetic construct
IYSGGST

SEQ ID NO: 852 - protein, 7 aa, synthetic construct
GFTVSSN

SEQ ID NO: 853 - protein, 5 aa, synthetic construct
YSGGS

SEQ ID NO: 854 - protein, 6 aa, synthetic construct
SSNYMS

SEQ ID NO: 855 - protein, 12 aa, synthetic construct
WVSVIYSGGSTY

SEQ ID NO: 856 - protein, 14 aa, synthetic construct
ARDRKIVVRRGGLD

SEQ ID NO: 857 - protein, 10 aa, synthetic construct
LLIYAASTLQ

SEQ ID NO: 858 - protein, 13 aa, synthetic construct
AASGFTFSSYSMN

SEQ ID NO: 859 - protein, 10 aa, synthetic construct
YISSSSSTIY

SEQ ID NO: 860 - protein, 11 aa, synthetic construct
ARDGAAAGVNP

SEQ ID NO: 861 - protein, 11 aa, synthetic construct
RASQSISSWLA

SEQ ID NO: 862 - protein, 8 aa, synthetic construct
YKASSLES

SEQ ID NO: 863 - protein, 9 aa, synthetic construct
QQYNSFPWT

SEQ ID NO: 864 - protein, 5 aa, synthetic construct
SYSMN

SEQ ID NO: 865 - protein, 17 aa, synthetic construct
YISSSSSTIYYADSVKG

SEQ ID NO: 866 - protein, 9 aa, synthetic construct
DGAAAGVNP

SEQ ID NO: 867 - protein, 7 aa, synthetic construct
KASSLES

SEQ ID NO: 868 - protein, 6 aa, synthetic construct
SSYSMN

SEQ ID NO: 869 - protein, 13 aa, synthetic construct
WVSYISSSSSTIY

SEQ ID NO: 870 - protein, 10 aa, synthetic construct
ARDGAAAGVN

SEQ ID NO: 871 - protein, 7 aa, synthetic construct
SSWLAWY

SEQ ID NO: 872 - protein, 10 aa, synthetic construct
LLIYKASSLE

SEQ ID NO: 873 - protein, 13 aa, synthetic construct
AASGFTFSGSAMH

SEQ ID NO: 874 - protein, 12 aa, synthetic construct
RIRSKANSYATA

SEQ ID NO: 875 - protein, 19 aa, synthetic construct
TRLTDIFRLLGSNFYGMDV

SEQ ID NO: 876 - protein, 11 aa, synthetic construct
QSADSSGTYVV

SEQ ID NO: 877 - protein, 5 aa, synthetic construct
GSAMH

SEQ ID NO: 878 - protein, 19 aa, synthetic construct
RIRSKANSYATAYAASVKG

SEQ ID NO: 879 - protein, 17 aa, synthetic construct
LTDIFRLLGSNFYGMDV

SEQ ID NO: 880 - protein, 8 aa, synthetic construct
GFTFSGSA

SEQ ID NO: 881 - protein, 10 aa, synthetic construct
IRSKANSYAT

SEQ ID NO: 882 - protein, 7 aa, synthetic construct
GFTFSGS

SEQ ID NO: 883 - protein, 8 aa, synthetic construct
RSKANSYA

SEQ ID NO: 884 - protein, 6 aa, synthetic construct
SGSAMH

SEQ ID NO: 885 - protein, 15 aa, synthetic construct
WVGRIRSKANSYATA

SEQ ID NO: 886 - protein, 18 aa, synthetic construct
TRLTDIFRLLGSNFYGMD

SEQ ID NO: 887 - protein, 10 aa, synthetic construct
QSADSSGTYV

SEQ ID NO: 888 - protein, 13 aa, synthetic construct
AASRFTFSRYAMT

SEQ ID NO: 889 - protein, 10 aa, synthetic construct
AISSTGDNPY

SEQ ID NO: 890 - protein, 18 aa, synthetic construct
AKDQGGADNNGYYYNFDY

SEQ ID NO: 891 - protein, 11 aa, synthetic construct
GGNDIGSKSVH

SEQ ID NO: 892 - protein, 11 aa, synthetic construct
QVWDSSSDHVV

SEQ ID NO: 893 - protein, 5 aa, synthetic construct
RYAMT

SEQ ID NO: 894 - protein, 17 aa, synthetic construct
AISSTGDNPYYADSVKG

SEQ ID NO: 895 - protein, 16 aa, synthetic construct
DQGGADNNGYYYNFDY

SEQ ID NO: 896 - protein, 8 aa, synthetic construct
RFTFSRYA

SEQ ID NO: 897 - protein, 8 aa, synthetic construct
ISSTGDNP

SEQ ID NO: 898 - protein, 6 aa, synthetic construct
DIGSKS

SEQ ID NO: 899 - protein, 7 aa, synthetic construct
RFTFSRY

SEQ ID NO: 900 - protein, 6 aa, synthetic construct
SSTGDN

SEQ ID NO: 901 - protein, 6 aa, synthetic construct
SRYAMT

SEQ ID NO: 902 - protein, 13 aa, synthetic construct
WVSAISSTGDNPY

SEQ ID NO: 903 - protein, 17 aa, synthetic construct
AKDQGGADNNGYYYNFD

SEQ ID NO: 904 - protein, 10 aa, synthetic construct
QVIYYDSDRP

SEQ ID NO: 905 - protein, 10 aa, synthetic construct
QVWDSSSDHV

SEQ ID NO: 906 - protein, 13 aa, synthetic construct
AASGFSFSTYAMN

SEQ ID NO: 907 - protein, 10 aa, synthetic construct
LFSGSGGDTY

SEQ ID NO: 908 - protein, 14 aa, synthetic construct
AKDLGVMFFGAFDI

SEQ ID NO: 909 - protein, 8 aa, synthetic construct
YKASSLQS

SEQ ID NO: 910 - protein, 9 aa, synthetic construct
QQYNDYSVT

SEQ ID NO: 911 - protein, 5 aa, synthetic construct
TYAMN

SEQ ID NO: 912 - protein, 17 aa, synthetic construct
LFSGSGGDTYYADSVKG

SEQ ID NO: 913 - protein, 12 aa, synthetic construct
DLGVMFFGAFDI

SEQ ID NO: 914 - protein, 7 aa, synthetic construct
KASSLQS

SEQ ID NO: 915 - protein, 8 aa, synthetic construct
GFSFSTYA

SEQ ID NO: 916 - protein, 8 aa, synthetic construct
FSGSGGDT

SEQ ID NO: 917 - protein, 7 aa, synthetic construct
GFSFSTY

SEQ ID NO: 918 - protein, 6 aa, synthetic construct
SGSGGD

SEQ ID NO: 919 - protein, 6 aa, synthetic construct
STYAMN

SEQ ID NO: 920 - protein, 13 aa, synthetic construct
WVSLFSGSGGDTY

SEQ ID NO: 921 - protein, 13 aa, synthetic construct
AKDLGVMFFGAFD

SEQ ID NO: 922 - protein, 10 aa, synthetic construct
LLIYKASSLQ

SEQ ID NO: 923 - protein, 8 aa, synthetic construct
QQYNDYSV

SEQ ID NO: 924 - protein, 13 aa, synthetic construct
AASGFTFTNYWMH

SEQ ID NO: 925 - protein, 10 aa, synthetic construct
RLNSDGSITG

SEQ ID NO: 926 - protein, 16 aa, synthetic construct
ARVGLRTLPAIENFDY

SEQ ID NO: 927 - protein, 11 aa, synthetic construct
RASQSISIYLN

SEQ ID NO: 928 - protein, 9 aa, synthetic construct
QQSYRAVLT

SEQ ID NO: 929 - protein, 5 aa, synthetic construct
NYWMH

SEQ ID NO: 930 - protein, 17 aa, synthetic construct
RLNSDGSITGYADSVKG

SEQ ID NO: 931 - protein, 14 aa, synthetic construct
VGLRTLPAIENFDY

SEQ ID NO: 932 - protein, 8 aa, synthetic construct
GFTFTNYW

SEQ ID NO: 933 - protein, 8 aa, synthetic construct
LNSDGSIT

SEQ ID NO: 934 - protein, 16 aa, synthetic construct
ARVGLRTLPAIENFDY

SEQ ID NO: 935 - protein, 6 aa, synthetic construct
QSISIY

SEQ ID NO: 936 - protein, 7 aa, synthetic construct
GFTFTNY

SEQ ID NO: 937 - protein, 6 aa, synthetic construct
NSDGSI

SEQ ID NO: 938 - protein, 6 aa, synthetic construct
TNYWMH

SEQ ID NO: 939 - protein, 13 aa, synthetic construct
WVSRLNSDGSITG

SEQ ID NO: 940 - protein, 15 aa, synthetic construct
ARVGLRTLPAIENFD

SEQ ID NO: 941 - protein, 7 aa, synthetic construct
SIYLNWY

SEQ ID NO: 942 - protein, 10 aa, synthetic construct
LLIYAASSLQ

SEQ ID NO: 943 - protein, 8 aa, synthetic construct
QQSYRAVL

SEQ ID NO: 944 - protein, 13 aa, synthetic construct
AASGFIFSYYWMS

SEQ ID NO: 945 - protein, 10 aa, synthetic construct
NIKEDGSEKY

SEQ ID NO: 946 - protein, 14 aa, synthetic construct
ARAPLRNFDWPLDV

SEQ ID NO: 947 - protein, 14 aa, synthetic construct
TGTSSDVGANDYVS

SEQ ID NO: 948 - protein, 8 aa, synthetic construct
YDVSKRTS

SEQ ID NO: 949 - protein, 8 aa, synthetic construct
CSYGGRYV

SEQ ID NO: 950 - protein, 5 aa, synthetic construct
YYWMS

SEQ ID NO: 951 - protein, 17 aa, synthetic construct
NIKEDGSEKYYVDSVKG

SEQ ID NO: 952 - protein, 12 aa, synthetic construct
APLRNFDWPLDV

SEQ ID NO: 953 - protein, 7 aa, synthetic construct
DVSKRTS

SEQ ID NO: 954 - protein, 8 aa, synthetic construct
GFIFSYYW

SEQ ID NO: 955 - protein, 8 aa, synthetic construct
IKEDGSEK

SEQ ID NO: 956 - protein, 9 aa, synthetic construct
SSDVGANDY

SEQ ID NO: 957 - protein, 7 aa, synthetic construct
GFIFSYY

SEQ ID NO: 958 - protein, 6 aa, synthetic construct
KEDGSE

SEQ ID NO: 959 - protein, 6 aa, synthetic construct
SYYWMS

SEQ ID NO: 960 - protein, 13 aa, synthetic construct
WVANIKEDGSEKY

SEQ ID NO: 961 - protein, 13 aa, synthetic construct
ARAPLRNFDWPLD

SEQ ID NO: 962 - protein, 10 aa, synthetic construct
VGANDYVSWY

SEQ ID NO: 963 - protein, 10 aa, synthetic construct
LIIYDVSKRT

SEQ ID NO: 964 - protein, 13 aa, synthetic construct
KGSGYKFSSYWIA

SEQ ID NO: 965 - protein, 8 aa, synthetic construct
VRGGSAPH

SEQ ID NO: 966 - protein, 17 aa, synthetic construct
KSSQSVLYSSNNKNYLT

SEQ ID NO: 967 - protein, 8 aa, synthetic construct
YWASTRES

SEQ ID NO: 968 - protein, 9 aa, synthetic construct
QQHYTTPWT

SEQ ID NO: 969 - protein, 5 aa, synthetic construct
SYWIA

SEQ ID NO: 970 - protein, 6 aa, synthetic construct
GGSAPH

SEQ ID NO: 971 - protein, 7 aa, synthetic construct
WASTRES

SEQ ID NO: 972 - protein, 8 aa, synthetic construct
GYKFSSYW

SEQ ID NO: 973 - protein, 12 aa, synthetic construct
QSVLYSSNNKNY

SEQ ID NO: 974 - protein, 7 aa, synthetic construct
GYKFSSY

SEQ ID NO: 975 - protein, 6 aa, synthetic construct
SSYWIA

SEQ ID NO: 976 - protein, 7 aa, synthetic construct
VRGGSAP

SEQ ID NO: 977 - protein, 13 aa, synthetic construct
LYSSNNKNYLTWY

SEQ ID NO: 978 - protein, 10 aa, synthetic construct
LLIYWASTRE

SEQ ID NO: 979 - protein, 8 aa, synthetic construct
QQHYTTPW

SEQ ID NO: 980 - protein, 13 aa, synthetic construct
KASGYTFNTFYMH

SEQ ID NO: 981 - protein, 10 aa, synthetic construct
VINPSRGRTI

SEQ ID NO: 982 - protein, 11 aa, synthetic construct
QSADRSDIYVV

SEQ ID NO: 983 - protein, 5 aa, synthetic construct
TFYMH

SEQ ID NO: 984 - protein, 17 aa, synthetic construct
VINPSRGRTIYAQKFQG

SEQ ID NO: 985 - protein, 8 aa, synthetic construct
GYTFNTFY

SEQ ID NO: 986 - protein, 8 aa, synthetic construct
INPSRGRT

SEQ ID NO: 987 - protein, 7 aa, synthetic construct
GYTFNTF

SEQ ID NO: 988 - protein, 6 aa, synthetic construct
NPSRGR

SEQ ID NO: 989 - protein, 6 aa, synthetic construct
NTFYMH

SEQ ID NO: 990 - protein, 13 aa, synthetic construct
WVGVINPSRGRTI

SEQ ID NO: 991 - DNA, 24 bp, synthetic construct
  source 1..24: mol_type = other DNA
ggagtcaccttcagtcgttttgac

SEQ ID NO: 992 - DNA, 24 bp, synthetic construct
  source 1..24: mol_type = other DNA
attagtagtagtggtactaccata

SEQ ID NO: 993 - DNA, 39 bp, synthetic construct
  source 1..39: mol_type = other DNA
gcgagagatgattacactggctggtactactttgactcc

SEQ ID NO: 994 - DNA, 27 bp, synthetic construct
  source 1..27: mol_type = other DNA
agctccaacatcggggcaggttttgac

SEQ ID NO: 995 - skipped

SEQ ID NO: 996 - DNA, 27 bp, synthetic construct
  source 1..27: mol_type = other DNA
cagtcctatgacagcagcctgtatgtc

SEQ ID NO: 997 - DNA, 30 bp, synthetic construct
  source 1..30: mol_type = other DNA
gggttctcactcagcactagtggaatgtgt

SEQ ID NO: 998 - DNA, 21 bp, synthetic construct
  source 1..21: mol_type = other DNA
attgattgggatgatgataaa

SEQ ID NO: 999 - DNA, 36 bp, synthetic construct
  source 1..36: mol_type = other DNA
gcacgccaggaactagtgggagcctggttcgacccc

SEQ ID NO: 1000 - DNA, 27 bp, synthetic construct
  source 1..27: mol_type = other DNA
agcggtgacgttggtggttataattat

SEQ ID NO: 1001 - skipped

SEQ ID NO: 1002 - DNA, 33 bp, synthetic construct
  source 1..33: mol_type = other DNA
atctcatatacaagcagcagcatctcttgtgtc

SEQ ID NO: 1003 - DNA, 24 bp, synthetic construct
  source 1..24: mol_type = other DNA
ggattcaccttcagtagctatggc

SEQ ID NO: 1004 - DNA, 24 bp, synthetic construct
  source 1..24: mol_type = other DNA
atatcatatgatggaagtactaaa

SEQ ID NO: 1005 - DNA, 42 bp, synthetic construct
  source 1..42: mol_type = other DNA
gcgaaagatggcacagcagtgcttgtggaccgtcttgactac

SEQ ID NO: 1006 - DNA, 18 bp, synthetic construct
  source 1..18: mol_type = other DNA
cagggcattagcagttat

SEQ ID NO: 1007 - skipped

SEQ ID NO: 1008 - protein, 27 aa, synthetic construct
CAACAGCTTAATAGTTACCCGCTCACT

SEQ ID NO: 1009 - protein, 24 aa, synthetic construct
GGATACGACTTTGACACGTACTGG

SEQ ID NO: 1010 - protein, 24 aa, synthetic construct
ATTTATCCTGATGACTCTGATACC

SEQ ID NO: 1011-1110 - skipped (100 entries)

SEQ ID NO: 1111 - DNA, 21 bp, synthetic construct
  source 1..21: mol_type = other DNA
gcgagagcaactggggactat

SEQ ID NO: 1112 - DNA, 18 bp, synthetic construct
  source 1..18: mol_type = other DNA
caaaatgttagtacttgg

SEQ ID NO: 1113 - skipped

SEQ ID NO: 1114 - DNA, 27 bp, synthetic construct
  source 1..27: mol_type = other DNA
caacaatatagaagttattctcgaacc

SEQ ID NO: 1115 - DNA, 24 bp, synthetic construct
  source 1..24: mol_type = other DNA
ggatacgactttaacacctactgg

SEQ ID NO: 1116 - DNA, 24 bp, synthetic construct
  source 1..24: mol_type = other DNA
atctaccctgatgactctgatacc

SEQ ID NO: 1117 - DNA, 18 bp, synthetic construct
  source 1..18: mol_type = other DNA
caaaatgttagtagatgg

SEQ ID NO: 1118 - DNA, 27 bp, synthetic construct
  source 1..27: mol_type = other DNA
caacaatataggagttattctcgaact

SEQ ID NO: 1119 - DNA, 24 bp, synthetic construct
  source 1..24: mol_type = other DNA
ggagccaccttcagcagctatgct

SEQ ID NO: 1120 - DNA, 24 bp, synthetic construct
  source 1..24: mol_type = other DNA
gtcacccctattattgatacaaca

SEQ ID NO: 1121 - DNA, 45 bp, synthetic construct
  source 1..45: mol_type = other DNA
gcgacgcgtgggacgtattacgatttttggcccagtatggacgtc

SEQ ID NO: 1122 - DNA, 18 bp, synthetic construct
  source 1..18: mol_type = other DNA
caggacattaggaattat

SEQ ID NO: 1123 - DNA, 27 bp, synthetic construct
  source 1..27: mol_type = other DNA
caacagtataatagttaccctctcact

SEQ ID NO: 1124 - DNA, 24 bp, synthetic construct
  source 1..24: mol_type = other DNA
ggtgggtccttcagtggttactac

SEQ ID NO: 1125 - DNA, 21 bp, synthetic construct
  source 1..21: mol_type = other DNA
atcaatcatagtggaagcacc

SEQ ID NO: 1126 - DNA, 39 bp, synthetic construct
  source 1..39: mol_type = other DNA
gcgagcatgagtgggagctactacggggcctttgactac

SEQ ID NO: 1127 - DNA, 18 bp, synthetic construct
  source 1..18: mol_type = other DNA
gcattgccaaagcaatat

SEQ ID NO: 1128 - skipped

SEQ ID NO: 1129 - DNA, 33 bp, synthetic construct
  source 1..33: mol_type = other DNA
caatcagcagacagcagtggtacttattatgtc

SEQ ID NO: 1130 - DNA, 27 bp, synthetic construct
  source 1..27: mol_type = other DNA
agtggcatcaatgttggtacctacagg

SEQ ID NO: 1131 - DNA, 21 bp, synthetic construct
  source 1..21: mol_type = other DNA
tacaaatcagactcagataag

SEQ ID NO: 1132 - DNA, 27 bp, synthetic construct
  source 1..27: mol_type = other DNA
atgatttggcacagcagcgcttgggtg

SEQ ID NO: 1133 - DNA, 24 bp, synthetic construct
  source 1..24: mol_type = other DNA
ggatttaccttcaagcactattct

SEQ ID NO: 1134 - DNA, 24 bp, synthetic construct
  source 1..24: mol_type = other DNA
atatcttatgatggaagcaagacg

SEQ ID NO: 1135 - DNA, 42 bp, synthetic construct
  source 1..42: mol_type = other DNA
gtgagagatcggacgactggccacgatttcggctttgactac

SEQ ID NO: 1136 - DNA, 18 bp, synthetic construct
  source 1..18: mol_type = other DNA
gcattgtcgaagaaatat

SEQ ID NO: 1137 - skipped

SEQ ID NO: 1138 - DNA, 33 bp, synthetic construct
  source 1..33: mol_type = other DNA
caatcaacagacagcgatacttcttatgtaatt

SEQ ID NO: 1139 - DNA, 24 bp, synthetic construct
  source 1..24: mol_type = other DNA
ggaggcttcttcagcagctatcgt

SEQ ID NO: 1140 - DNA, 24 bp, synthetic construct
  source 1..24: mol_type = other DNA
ctcgtccctattttcgaaacagcc

SEQ ID NO: 1141 - DNA, 39 bp, synthetic construct
  source 1..39: mol_type = other DNA
gcgagagggtcgggcagtgggcttacatacttccaaacc

SEQ ID NO: 1142 - DNA, 21 bp, synthetic construct
  source 1..21: mol_type = other DNA
cagagtattaccacctactac

SEQ ID NO: 1143 - skipped

SEQ ID NO: 1144 - DNA, 24 bp, synthetic construct
  source 1..24: mol_type = other DNA
cagcagtatgatagttcacccacg

SEQ ID NO: 1145 - DNA, 30 bp, synthetic construct
  source 1..30: mol_type = other DNA
ggtggctccatcaccagtattagtcactac

SEQ ID NO: 1146 - DNA, 21 bp, synthetic construct
  source 1..21: mol_type = other DNA
atgtattacagtgggaccacc

SEQ ID NO: 1147 - DNA, 30 bp, synthetic construct
  source 1..30: mol_type = other DNA
gcgagacatgacgttagcaggttcgacccc

SEQ ID NO: 1148 - DNA, 27 bp, synthetic construct
  source 1..27: mol_type = other DNA
agcagtgacgttggtggttataactat

SEQ ID NO: 1149 - skipped

SEQ ID NO: 1150 - DNA, 27 bp, synthetic construct
  source 1..27: mol_type = other DNA
agctcatatacaaccagcacttgggtg

SEQ ID NO: 1151 - DNA, 24 bp, synthetic construct
  source 1..24: mol_type = other DNA
ggattcaccttcagtaattttgat

SEQ ID NO: 1152 - DNA, 24 bp, synthetic construct
  source 1..24: mol_type = other DNA
atttcatatgatgaaagtaaaaga

SEQ ID NO: 1153 - DNA, 39 bp, synthetic construct
  source 1..39: mol_type = other DNA
gcgaccgagtccacgcaatactttggtgctcttgatacc

SEQ ID NO: 1154 - DNA, 18 bp, synthetic construct
  source 1..18: mol_type = other DNA
ggattggcagaccaatat

SEQ ID NO: 1155 - skipped

SEQ ID NO: 1156 - DNA, 30 bp, synthetic construct
  source 1..30: mol_type = other DNA
caatcaacagacagaagtggctcttatcta

SEQ ID NO: 1157 - DNA, 24 bp, synthetic construct
  source 1..24: mol_type = other DNA
ggattcaacttcaatttttacact

SEQ ID NO: 1158 - DNA, 24 bp, synthetic construct
  source 1..24: mol_type = other DNA
gtattttatgatgggagcaaaacc

SEQ ID NO: 1159 - DNA, 42 bp, synthetic construct
  source 1..42: mol_type = other DNA
gtgagagatagaacgacaggacacgattacggcatggacgtc

SEQ ID NO: 1160 - DNA, 18 bp, synthetic construct
  source 1..18: mol_type = other DNA
ggcttggcaaacctatat

SEQ ID NO: 1161 - skipped

SEQ ID NO: 1162 - DNA, 33 bp, synthetic construct
  source 1..33: mol_type = other DNA
caatcagcagacagcagtcgttcttctgtgatc

SEQ ID NO: 1163 - DNA, 30 bp, synthetic construct
  source 1..30: mol_type = other DNA
gggttctcactcagcactggtggagtgtgt

SEQ ID NO: 1164 - DNA, 21 bp, synthetic construct
  source 1..21: mol_type = other DNA
attgattgggataatgataaa

SEQ ID NO: 1165 - DNA, 48 bp, synthetic construct
  source 1..48: mol_type = other DNA
gcacgcatgacgggcctgtatagtgacaactggaagggttttgatatc

SEQ ID NO: 1166 - DNA, 21 bp, synthetic construct
  source 1..21: mol_type = other DNA
cagagtgttgacagcacttac

SEQ ID NO: 1167 - skipped

SEQ ID NO: 1168 - DNA, 30 bp, synthetic construct
  source 1..30: mol_type = other DNA
cagcagtatggtagcgcaccggggtacact

SEQ ID NO: 1169 - DNA, 24 bp, synthetic construct
  source 1..24: mol_type = other DNA
ggatacagatttaccagttactgg

SEQ ID NO: 1170 - DNA, 24 bp, synthetic construct
  source 1..24: mol_type = other DNA
atctatcctcctgactctgatact

SEQ ID NO: 1171 - DNA, 27 bp, synthetic construct
  source 1..27: mol_type = other DNA
gcgagacagggaaggggatttgactac

SEQ ID NO: 1172 - DNA, 18 bp, synthetic construct
  source 1..18: mol_type = other DNA
caaaacatttacacttat

SEQ ID NO: 1173 - skipped

SEQ ID NO: 1174 - DNA, 27 bp, synthetic construct
  source 1..27: mol_type = other DNA
cagcagagttacgattcccctcgaacg

SEQ ID NO: 1175 - DNA, 30 bp, synthetic construct
  source 1..30: mol_type = other DNA
gggttctcactcaacactgctgaagtgtgt

SEQ ID NO: 1176 - DNA, 48 bp, synthetic construct
  source 1..48: mol_type = other DNA
gcacgcatgacgggcctgtatactgacaactggaagggttttgatatg

SEQ ID NO: 1177 - DNA, 21 bp, synthetic construct
  source 1..21: mol_type = other DNA
cagactattgacagtacttac

SEQ ID NO: 1178 - DNA, 30 bp, synthetic construct
  source 1..30: mol_type = other DNA
cagcagtatggtcacgcaccggggtacact

SEQ ID NO: 1179 - DNA, 24 bp, synthetic construct
  source 1..24: mol_type = other DNA
ggaggcaccttcagtagatatgct

SEQ ID NO: 1180 - DNA, 24 bp, synthetic construct
  source 1..24: mol_type = other DNA
atcatccctatgtttgatataaca

SEQ ID NO: 1181 - DNA, 57 bp, synthetic construct
  source 1..57: mol_type = other DNA
gcgactgagagttgtagtggtggtaactgctattgggatcatgatgcttttgatatc

SEQ ID NO: 1182 - DNA, 21 bp, synthetic construct
  source 1..21: mol_type = other DNA
cagagtcttagcagcagctac

SEQ ID NO: 1183 - DNA, 30 bp, synthetic construct
  source 1..30: mol_type = other DNA
cagcagtatggcaggtcacttccgaggacg

SEQ ID NO: 1184 - DNA, 24 bp, synthetic construct
  source 1..24: mol_type = other DNA
ggaggcaccttcagcagctatgct

SEQ ID NO: 1185 - DNA, 24 bp, synthetic construct
  source 1..24: mol_type = other DNA
atcattcctatctttgatatagca

SEQ ID NO: 1186 - DNA, 57 bp, synthetic construct
  source 1..57: mol_type = other DNA
gcgagtgagaattgtggcggtggtaactgttactgggagcatgatgcttttgacatc

SEQ ID NO: 1187 - DNA, 21 bp, synthetic construct
  source 1..21: mol_type = other DNA
cagagtgttagcggcggcgac

SEQ ID NO: 1188 - DNA, 30 bp, synthetic construct
  source 1..30: mol_type = other DNA
cagcactatggtaattcacctccgaggacg

SEQ ID NO: 1189 - DNA, 24 bp, synthetic construct
  source 1..24: mol_type = other DNA
ggaggcaccttcagcacctatgct

SEQ ID NO: 1190 - DNA, 57 bp, synthetic construct
  source 1..57: mol_type = other DNA
gcgagtgagaattgtagcggtggtaactgttactgggaacatgatgcttttgacatc

SEQ ID NO: 1191 - DNA, 30 bp, synthetic construct
  source 1..30: mol_type = other DNA
cagcagtatggtaattcacctccgaggacg

SEQ ID NO: 1192 - DNA, 24 bp, synthetic construct
  source 1..24: mol_type = other DNA
ggatacagctttgccagtttctgg

SEQ ID NO: 1193 - DNA, 24 bp, synthetic construct
  source 1..24: mol_type = other DNA
atatatcctagtgactctgataca

SEQ ID NO: 1194 - DNA, 33 bp, synthetic construct
  source 1..33: mol_type = other DNA
gcgaaacatggagtcggagcagctattgaccac

SEQ ID NO: 1195 - DNA, 18 bp, synthetic construct
  source 1..18: mol_type = other DNA
caggtcattagcggctat

SEQ ID NO: 1196 - skipped

SEQ ID NO: 1197 - DNA, 18 bp, synthetic construct
  source 1..18: mol_type = other DNA
caggtcattagcggctat

SEQ ID NO: 1198 - DNA, 30 bp, synthetic construct
  source 1..30: mol_type = other DNA
ggtggctccattgggggtggtccttaccac

SEQ ID NO: 1199 - DNA, 21 bp, synthetic construct
  source 1..21: mol_type = other DNA
gtggaaaccagtggggacact

SEQ ID NO: 1200 - DNA, 33 bp, synthetic construct
  source 1..33: mol_type = other DNA
gcgagaggaacagctatggatgcttttgatatc

SEQ ID NO: 1201 - DNA, 18 bp, synthetic construct
  source 1..18: mol_type = other DNA
caggatattagtaactgg

SEQ ID NO: 1202 - skipped

SEQ ID NO: 1203 - DNA, 27 bp, synthetic construct
  source 1..27: mol_type = other DNA
caacagggtaacagtttcccgctcact

SEQ ID NO: 1204 - DNA, 24 bp, synthetic construct
  source 1..24: mol_type = other DNA
ggattcagcttcagtggctatggc

SEQ ID NO: 1205 - DNA, 24 bp, synthetic construct
  source 1..24: mol_type = other DNA
atttggtatgatggaagtagtaaa

SEQ ID NO: 1206 - DNA, 30 bp, synthetic construct
  source 1..30: mol_type = other DNA
gcgagagacgtcctcggggagttgcccgat

SEQ ID NO: 1207 - DNA, 18 bp, synthetic construct
  source 1..18: mol_type = other DNA
cagggcattagcaattct

SEQ ID NO: 1208 - skipped

SEQ ID NO: 1209 - DNA, 27 bp, synthetic construct
  source 1..27: mol_type = other DNA
caacagtataatacctaccctctcact

SEQ ID NO: 1210 - DNA, 24 bp, synthetic construct
  source 1..24: mol_type = other DNA
ggctttaccttcagtaattatggc

SEQ ID NO: 1211 - DNA, 21 bp, synthetic construct
  source 1..21: mol_type = other DNA
atatggtttgatggacgaaca

SEQ ID NO: 1212 - DNA, 42 bp, synthetic construct
  source 1..42: mol_type = other DNA
gcgagagaaacatggagttacgccgggggggtctttgactac

SEQ ID NO: 1213 - DNA, 21 bp, synthetic construct
  source 1..21: mol_type = other DNA
agtgggcacagcggtcacgcc

SEQ ID NO: 1214 - DNA, 21 bp, synthetic construct
  source 1..21: mol_type = other DNA
gttcacagtgatggcagccac

SEQ ID NO: 1215 - DNA, 27 bp, synthetic construct
  source 1..27: mol_type = other DNA
cagacctgggacactggcctttgggtg

SEQ ID NO: 1216 - DNA, 24 bp, synthetic construct
  source 1..24: mol_type = other DNA
ggatacaccttcaccacctactat

SEQ ID NO: 1217 - DNA, 24 bp, synthetic construct
  source 1..24: mol_type = other DNA
ctcaaccctagtcgtaataggact

SEQ ID NO: 1218 - DNA, 45 bp, synthetic construct
  source 1..45: mol_type = other DNA
gcgagagatgcgggactgggtccgtactactactacatggacgtc

SEQ ID NO: 1219 - DNA, 18 bp, synthetic construct
  source 1..18: mol_type = other DNA
ggattgacaaagcaatat

SEQ ID NO: 1220 - DNA, 33 bp, synthetic construct
  source 1..33: mol_type = other DNA
caatcagcagacagaactgatatttatgtggtc

SEQ ID NO: 1221 - DNA, 24 bp, synthetic construct
  source 1..24: mol_type = other DNA
ggaggcaccttcagcagcgctact

SEQ ID NO: 1222 - DNA, 24 bp, synthetic construct
  source 1..24: mol_type = other DNA
atcacccctatgtttactacagca

SEQ ID NO: 1223 - DNA, 45 bp, synthetic construct
  source 1..45: mol_type = other DNA
gcgaggcaatttggtcatgaaagtcgtgattcttactacctctac

SEQ ID NO: 1224 - DNA, 21 bp, synthetic construct
  source 1..21: mol_type = other DNA
cagactcttagcagcagctac

SEQ ID NO: 1225 - DNA, 24 bp, synthetic construct
  source 1..24: mol_type = other DNA
cagcaatatggtagttcgcggacg

SEQ ID NO: 1226 - DNA, 27 bp, synthetic construct
  source 1..27: mol_type = other DNA
ggttactccatcagcagtggttactac

SEQ ID NO: 1227 - DNA, 21 bp, synthetic construct
  source 1..21: mol_type = other DNA
atctatcatagtgggagcacc

SEQ ID NO: 1228 - DNA, 42 bp, synthetic construct
  source 1..42: mol_type = other DNA
gcgagatctgcaagagcagtggttatggatgcttttcatatc

SEQ ID NO: 1229 - DNA, 24 bp, synthetic construct
  source 1..24: mol_type = other DNA
agtggcagcattgccagcaactat

SEQ ID NO: 1230 - skipped

SEQ ID NO: 1231 - DNA, 30 bp, synthetic construct
  source 1..30: mol_type = other DNA
cagtcttatgataatatccatccttgggtg

SEQ ID NO: 1232 - DNA, 18 bp, synthetic construct
  source 1..18: mol_type = other DNA
aacattgggagtaaaagt

SEQ ID NO: 1233 - skipped

SEQ ID NO: 1234 - DNA, 36 bp, synthetic construct
  source 1..36: mol_type = other DNA
caggtgtgggatggtagtactgatcatttttatgtc

SEQ ID NO: 1235 - DNA, 30 bp, synthetic construct
  source 1..30: mol_type = other DNA
ggtggctccatcagcagtggtgaatactac

SEQ ID NO: 1236 - DNA, 21 bp, synthetic construct
  source 1..21: mol_type = other DNA
atctcttacgccgggaatacc

SEQ ID NO: 1237 - DNA, 51 bp, synthetic construct
  source 1..51: mol_type = other DNA
gccagagatttcgttggcaccaactggtacgacagatatgcttttcatatc

SEQ ID NO: 1238 - DNA, 27 bp, synthetic construct
  source 1..27: mol_type = other DNA
agcagtgatgttggtggttatcactat

SEQ ID NO: 1239 - DNA, 33 bp, synthetic construct
  source 1..33: mol_type = other DNA
tgctcatatgcaggcagctacgctttttgggtt

SEQ ID NO: 1240 - DNA, 24 bp, synthetic construct
  source 1..24: mol_type = other DNA
ggattcaatttcatcgactacgct

SEQ ID NO: 1241 - DNA, 24 bp, synthetic construct
  source 1..24: mol_type = other DNA
attactaatgatgcgaaaagcaca

SEQ ID NO: 1242 - DNA, 45 bp, synthetic construct
  source 1..45: mol_type = other DNA
gcgagaggccgcagtcattattatgatagtagtggatttgactac

SEQ ID NO: 1243 - DNA, 18 bp, synthetic construct
  source 1..18: mol_type = other DNA
agccttggcagtaaaagt

SEQ ID NO: 1244 - skipped

SEQ ID NO: 1245 - DNA, 30 bp, synthetic construct
  source 1..30: mol_type = other DNA
caggtgtgggatgctggtgccctttgggtg

SEQ ID NO: 1246 - DNA, 30 bp, synthetic construct
  source 1..30: mol_type = other DNA
ggcgactccatcagcactacttcttactac

SEQ ID NO: 1247 - DNA, 21 bp, synthetic construct
  source 1..21: mol_type = other DNA
atcaattatagtggcaacacc

SEQ ID NO: 1248 - DNA, 48 bp, synthetic construct
  source 1..48: mol_type = other DNA
gcgagacggggggtcccagctggtatggtcccgtcctggatcgacccc

SEQ ID NO: 1249 - DNA, 18 bp, synthetic construct
  source 1..18: mol_type = other DNA
gaggacattcgcaactat

SEQ ID NO: 1250 - skipped

SEQ ID NO: 1251 - DNA, 27 bp, synthetic construct
  source 1..27: mol_type = other DNA
caacaatatgatactgtccctctcact

SEQ ID NO: 1252 - DNA, 24 bp, synthetic construct
  source 1..24: mol_type = other DNA
ggataccactttcccacctactgg

SEQ ID NO: 1253 - DNA, 24 bp, synthetic construct
  source 1..24: mol_type = other DNA
atctatcctcgtgactccgatacc

SEQ ID NO: 1254 - DNA, 21 bp, synthetic construct
  source 1..21: mol_type = other DNA
gcgagagcaactggggactac

SEQ ID NO: 1255 - DNA, 18 bp, synthetic construct
  source 1..18: mol_type = other DNA
gagaatgttagtacctgg

SEQ ID NO: 1256 - skipped

SEQ ID NO: 1257 - DNA, 27 bp, synthetic construct
  source 1..27: mol_type = other DNA
caacaatataagagttattctcgaacg

SEQ ID NO: 1258 - DNA, 24 bp, synthetic construct
  source 1..24: mol_type = other DNA
ggatacagtttccccacctactgg

SEQ ID NO: 1259 - DNA, 24 bp, synthetic construct
  source 1..24: mol_type = other DNA
atctatccagatgactccgatacc

SEQ ID NO: 1260 - DNA, 21 bp, synthetic construct
  source 1..21: mol_type = other DNA
gcgcgaggtatgggggatcac

SEQ ID NO: 1261 - DNA, 33 bp, synthetic construct
  source 1..33: mol_type = other DNA
cagagcctcctctttggtgatggagcgacctat

SEQ ID NO: 1262 - skipped

SEQ ID NO: 1263 - DNA, 27 bp, synthetic construct
  source 1..27: mol_type = other DNA
atgcaagctctacatatgcctcggacg

SEQ ID NO: 1264 - DNA, 24 bp, synthetic construct
  source 1..24: mol_type = other DNA
ggattcaccgtcagtagcaactac

SEQ ID NO: 1265 - DNA, 21 bp, synthetic construct
  source 1..21: mol_type = other DNA
atttatagcggtggtagcaca

SEQ ID NO: 1266 - DNA, 45 bp, synthetic construct
  source 1..45: mol_type = other DNA
gcgagagatcggaagatagtagttaggcgcgggggcttagatatc

SEQ ID NO: 1267 - DNA, 18 bp, synthetic construct
  source 1..18: mol_type = other DNA
cagggtattagcagttat

SEQ ID NO: 1268 - DNA, 27 bp, synthetic construct
  source 1..27: mol_type = other DNA
caacagtattatagttaccctcacacc

SEQ ID NO: 1269 - DNA, 24 bp, synthetic construct
  source 1..24: mol_type = other DNA
ggattcaccttcagtagctatagc

SEQ ID NO: 1270 - DNA, 24 bp, synthetic construct
  source 1..24: mol_type = other DNA
attagtagtagtagtagtaccata

SEQ ID NO: 1271 - DNA, 33 bp, synthetic construct
  source 1..33: mol_type = other DNA
gcgagagatggggcagcagctggggtcaacccc

SEQ ID NO: 1272 - DNA, 18 bp, synthetic construct
  source 1..18: mol_type = other DNA
cagagtattagtagctgg

SEQ ID NO: 1273 - DNA, 27 bp, synthetic construct
  source 1..27: mol_type = other DNA
caacagtataatagtttcccgtggacg

SEQ ID NO: 1274 - DNA, 24 bp, synthetic construct
  source 1..24: mol_type = other DNA
gggttcaccttcagtggctctgct

SEQ ID NO: 1275 - DNA, 30 bp, synthetic construct
  source 1..30: mol_type = other DNA
attagaagcaaagctaacagttacgcgaca

SEQ ID NO: 1276 - DNA, 57 bp, synthetic construct
  source 1..57: mol_type = other DNA
actagactaaccgatatttttcgactgctgggcagcaacttttacggtatggacgtc

SEQ ID NO: 1277 - DNA, 33 bp, synthetic construct
  source 1..33: mol_type = other DNA
caatcagcagacagcagtggtacttatgtggta

SEQ ID NO: 1278 - DNA, 24 bp, synthetic construct
  source 1..24: mol_type = other DNA
cgattcacctttagccgctatgcc

SEQ ID NO: 1279 - DNA, 24 bp, synthetic construct
  source 1..24: mol_type = other DNA
attagcagcactggtgacaaccca

SEQ ID NO: 1280 - DNA, 54 bp, synthetic construct
  source 1..54: mol_type = other DNA
gcgaaagaccaggggggggctgataataatggttattactacaactttgactac

SEQ ID NO: 1281 - DNA, 18 bp, synthetic construct
  source 1..18: mol_type = other DNA
gacattggaagtaaaagt

SEQ ID NO: 1282 - DNA, 33 bp, synthetic construct
  source 1..33: mol_type = other DNA
caggtgtgggatagtagtagtgatcatgtggta

SEQ ID NO: 1283 - DNA, 24 bp, synthetic construct
  source 1..24: mol_type = other DNA
ggattcagctttagcacctatgcc

SEQ ID NO: 1284 - DNA, 24 bp, synthetic construct
  source 1..24: mol_type = other DNA
tttagtgggagtggtggtgacaca

SEQ ID NO: 1285 - DNA, 42 bp, synthetic construct
  source 1..42: mol_type = other DNA
gcgaaagatctaggggttatgtttttcggagcttttgatatc

SEQ ID NO: 1286 - DNA, 27 bp, synthetic construct
  source 1..27: mol_type = other DNA
caacagtataatgattattcggtcact

SEQ ID NO: 1287 - DNA, 24 bp, synthetic construct
  source 1..24: mol_type = other DNA
ggattcaccttcactaactactgg

SEQ ID NO: 1288 - DNA, 24 bp, synthetic construct
  source 1..24: mol_type = other DNA
cttaattctgatgggagtatcaca

SEQ ID NO: 1289 - DNA, 48 bp, synthetic construct
  source 1..48: mol_type = other DNA
gcaagagtaggtctacggactttgcccgcaatcgaaaactttgactac

SEQ ID NO: 1290 - DNA, 18 bp, synthetic construct
  source 1..18: mol_type = other DNA
cagagcattagcatctat

SEQ ID NO: 1291 - DNA, 27 bp, synthetic construct
  source 1..27: mol_type = other DNA
caacagagttacagggccgtgctcact

SEQ ID NO: 1292 - DNA, 24 bp, synthetic construct
  source 1..24: mol_type = other DNA
ggattcatctttagttactattgg

SEQ ID NO: 1293 - DNA, 24 bp, synthetic construct
  source 1..24: mol_type = other DNA
ataaaggaagacggaagtgagaag

SEQ ID NO: 1294 - DNA, 42 bp, synthetic construct
  source 1..42: mol_type = other DNA
gcgagggcgcccttacggaattttgactggccactcgacgtc

SEQ ID NO: 1295 - DNA, 27 bp, synthetic construct
  source 1..27: mol_type = other DNA
agcagtgatgttggtgctaatgactat

SEQ ID NO: 1296 - skipped

SEQ ID NO: 1297 - DNA, 24 bp, synthetic construct
  source 1..24: mol_type = other DNA
tgctcgtatgggggcaggtatgtc

SEQ ID NO: 1298 - DNA, 24 bp, synthetic construct
  source 1..24: mol_type = other DNA
ggatacaaatttagcagttattgg

SEQ ID NO: 1299 - DNA, 24 bp, synthetic construct
  source 1..24: mol_type = other DNA
atctatcctgatgactctgatacc

SEQ ID NO: 1300 - DNA, 24 bp, synthetic construct
  source 1..24: mol_type = other DNA
gtgagaggggggagtgccccccat

SEQ ID NO: 1301 - DNA, 36 bp, synthetic construct
  source 1..36: mol_type = other DNA
cagagtgttttatacagctccaacaataagaactac

SEQ ID NO: 1302 - skipped

SEQ ID NO: 1303 - DNA, 27 bp, synthetic construct
  source 1..27: mol_type = other DNA
caacaacattatactactccgtggacg

SEQ ID NO: 1304 - DNA, 24 bp, synthetic construct
  source 1..24: mol_type = other DNA
ggatacaccttcaacaccttttat

SEQ ID NO: 1305 - DNA, 24 bp, synthetic construct
  source 1..24: mol_type = other DNA
atcaaccctagtcgtggtaggaca

SEQ ID NO: 1306 - DNA, 45 bp, synthetic construct
  source 1..45: mol_type = other DNA
gcgagagatgcgggacttggtccgtactactactacatggacgtc

SEQ ID NO: 1307 - DNA, 33 bp, synthetic construct
  source 1..33: mol_type = other DNA
caatcagcagacagaagtgatatttatgtggta

SEQ ID NO: 1308 - protein, 10 aa, synthetic construct
QQYGRSLPRT

SEQ ID NO: 1309 - protein, 116 aa, synthetic construct
EVQLVQSGAEVKKPGESLKISCKVSGYRFTSYWIGWVRQMPGKGLEWMGVIYPPDSDTAYSPSFQGQVTMSVDNSISTAYLQWSNLKASDTAMYYCARQGRGFDYWGQGTLVTVSS

SEQ ID NO: 1310 - protein, 107 aa, synthetic construct
DIQVAQSPSSLSASVGDSVTITCRTSQNIYTYLNWYQQKPGKAPNLLINTASSLQGGVPSRFSGSGSGTDFTLTISSLRPEDFATYYCQQSYDSPRTFGQGTRVESK

SEQ ID NO: 1311 - protein, 126 aa, synthetic construct
QVQLVQSGAEVKKPGSSVKVSCKAYGGTFSTYAISWVRQAPGQGLEWMGGIIPIFDIANYAQKFQGRVTITADESTSTAFMELSSLRSEDTAVYYCASENCSGGNCYWEHDAFDIWGQGTRVTVSS

SEQ ID NO: 1312 - protein, 109 aa, synthetic construct
EIVLTQSPDTLSLSPGERATLSCRASQSVSGGDLAWYQQKPGQAPRLLMYGASIRATGIPDRFSGSGSGTDFTLTISRLEPEDFVVYYCQQYGNSPPRTFGQGTKVEIK

SEQ ID NO: 1313 - protein, 122 aa, synthetic construct
QVQLVHSGAEVKKPGASVKVSCKASGYTFTTYYIHWVRQAPGQGLEWVGVLNPSRNRTIYAQKFQGRVTMTRDTSTSTVYMELSSLRSEDTAVYYCARDAGLGPYYYYMDVWGKGTTVTVSS

SEQ ID NO: 1314 - protein, 108 aa, synthetic construct
SYELTQPPSVSVSPGQTARITCSGDGLTKQYAFWYQQKPGQAPVLVIYKDSERPSGIPERFSGSSSGTTVTLTISGVQAEDEADYYCQSADRTDIYVVFGGGTKLTVL

SEQ ID NO: 1315 - protein, 9 aa, synthetic construct
IAPNCYGNI

SEQ ID NO: 1316 - protein, 15 aa, synthetic construct
SSSGYGSFSASQASD

(19) United States
(12) Patent Application Publication
(10) Pub. No.: US 2024/0131132 A1
(43) Pub. Date: Apr. 25, 2024

(54) ANTIBODIES THAT BIND THE TUMOR (T) ANTIGEN OF MERKEL CELL POLYOMAVIRUS AND RELATED DIAGNOSTICS

(71) Applicants: Fred Hutchinson Cancer Center, Seattle, WA (US); University of Washington, Seattle, WA (US)

(72) Inventors: Justin J. Taylor, Seattle, WA (US); Denise Galloway, Seattle, WA (US); Paul Nghiem, Seattle, WA (US); David Koelle, Seattle, WA (US); Haroldo Rodriguez, Seattle, WA (US); Joseph Carter, Seattle, WA (US)

(73) Assignees: Fred Hutchinson Cancer Center, Seattle, WA (US); University of Washington, Seattle, WA (US)

(21) Appl. No.: 18/492,705

(22) Filed: Oct. 22, 2023

Related U.S. Application Data
(60) Provisional application No. 63/380,549, filed on Oct. 21, 2022.

(51) Int. Cl. A61K 39/00; A61K 45/06; A61P 35/00; C07K 16/28; G01N 33/533; G01N 33/574

(52) U.S. Cl. CPC A61K 39/001111; A61K 45/06; A61P 35/00; C07K 16/2809; G01N 33/533; G01N 33/57492; A61K 2039/53; G01N 2333/7051

(57) ABSTRACT

Antibodies that bind the tumor (T) antigen of the Merkel cell polyomavirus are disclosed. The antibodies can be use used in cell-based immunotherapies, antibody-based therapies, diagnostics, and detection assays, among other uses. Related diagnostics are also described

### CROSS-REFERENCE TO RELATED APPLICATION

This application claims priority to U.S. Provisional Patent Application No. 63/380,549 filed Oct. 21, 2022, which is incorporated herein by reference in its entirety as if fully set forth herein.

### STATEMENT REGARDING FEDERALLY SPONSORED RESEARCH OR DEVELOPMENT

This invention was made with government support under CA225517 awarded by the National Institutes of Health. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is 2ZK8452-ST26.xml. The file is 1,077,020 bytes, was created Oct. 23, 2023, and is being submitted electronically via Patent Center.

### FIELD OF THE DISCLOSURE

The current disclosure provides antibodies that bind the tumor (T) antigen of Merkel cell polyomavirus. The disclosed antibodies can be used for a variety of purposes including in cell-based immunotherapies, antibody-based therapies, and detection assays in the context of infection with Merkel cell polyomavirus and the associated Merkel cell carcinoma. Related diagnostics are also described.

### BACKGROUND OF THE DISCLOSURE

Merkel cell carcinoma (MCC) is a rare, aggressive skin cancer with a reported incidence of 2,000 new cases each year in the United States. The number of people diagnosed with MCC has been rising quickly over the past few decades. An increased risk of MCC has been linked with immunosuppression related to UV radiation, viral infections, organ transplantation, and chronic lymphocytic leukemia (Paulson et al., J. Invest. Dermatol. 729:1547, 2009; Goh et al., Oncotarget 7:3403, 2016; Feng et al., Science 379: 1096, 2008). While MCC is more frequently observed in immunocompromised or elderly populations, more than 90% of patients with MCC do not appear to be observably immune compromised (Heath et al., J. Am. Acad. Dermatol. 58: 375, 2008). Nonetheless, MCC is more lethal than melanoma with a reported 40% mortality rate (Heath et al., 2008). Once metastasized, MCC has an even worse prognosis. Only 18% of patients having stage IV metastatic disease survive for 5 years following diagnosis (Lemos and Nghiem, J Invest Dermatol. 127(9):2100-3, 2007).

Merkel cell polyomavirus (MCPyV) is associated with 80% of MCCs. MCPyV is a naked double-stranded DNA virus belonging to Polyomaviridae family. The early region of its circular genome includes the “Tumor” (T) antigen gene locus which encodes the large T (LT) and small T (sT) antigens. LT and sT are products of alternate reading frames of the LT open reading frame (ALTO) (Pietropaolo et al., Cancers (Basel) 2020, 12(7):1774). LT and sT share 78 amino acids at the amino-terminus (the common T antigen) and their expression appears to be necessary for MCC maintenance (Houben et al., J. Virol. 84: 7064, 2010).

### SUMMARY OF THE DISCLOSURE

The present disclosure describes antibodies that bind the common tumor (T) antigen of Merkel cell polyomavirus. The disclosed antibodies can be used in in cell-based immunotherapies, antibody-based therapies, and detection assays in the context of infection with Merkel cell polyomavirus and the associated Merkel cell carcinoma.

In particular embodiments, cell-based immunotherapies include B cells genetically modified to express an antibody disclosed herein. B cell activity in the tumor microenvironment has been shown to be correlated with positive clinical outcomes in the treatment of cancer. Embodiments disclosed herein also include cell-based therapies including other immune cells genetically modified to express a recombinant receptor, such as a chimeric antigen receptor (CAR) or an engineered T cell receptor (eTCR) having a binding domain of an antibody disclosed herein.

Antibody-based therapies can include, for example, “free” antibodies or antibody conjugates among numerous other therapeutic and detection formats disclosed elsewhere herein. For example, antibody conjugates can include an antibody or binding fragment thereof as a binding domain linked to an immunotoxin, drug, detectable label, and/or radioisotope.

In particular embodiments, diagnostic assays include antibodies or binding fragments thereof used in a platform for detecting the presence of the common T antigen of the Merkel cell polyomavirus in a sample. In particular embodiments, the antibodies or binding fragments thereof are immobilized on a solid support to form, for example, immunobeads, immunoassays, and lateral flow assays. In certain examples, antibodies or binding fragments thereof are used to provide a control level in an assay.

### BRIEF DESCRIPTION OF THE FIGURES

Some of the drawings submitted herewith may be better understood in color. Applicant considers the color versions of the drawings as part of the original submission and reserves the right to present color images of the drawings in later proceedings.

FIG. 1. MCPyV-derived T antigen (T-Ag) drives Virus-Positive (VP)-MCC (FIG. created in BioRender).

FIG. 2. Model of Immune Control of MCC (FIG. created in BioRender).

FIG. 3. Lack of detectable T-Ag-specific serum antibodies near time of VP-MCC diagnosis trends towards worse outcome.

FIGS. 4A-4D. T-Ag tetramers detect murine and human T-Ag-specific B cells. (4A) Analysis of T-Ag tetramer-binding B cells in pooled spleen and lymph node samples from C57BL/6 mice with and without subcutaneous injection of T-Ag in CFA seven days prior. Tetramer binding B cells are enriched prior to analysis using magnetic microbeads specific for PE or APC and samples are labeled with control tetramers to exclude B cells binding streptavidin, fluorochrome or the GST purification tag. (4B) Detection of B220LOW Intracellular IgHIGH antibody secreting cells and B220HIGH Intracellular IgMID GL7+CD38LOW germinal center within the population of T-Ag tetramer+cells. (4C) Analysis of T-Ag tetramer binding B cells from three concatenated PBMC samples from MCC patients. (4D) % of T-Ag tetramer-binding B cells in 4 healthy control and 5 MCC patient PBMC samples at the indicated time before or after tumor removal surgery. Lines connect time points for the same patient.

FIGS. 5A-5D. (5A) Three patients or three control PBMC samples were collected and each sample was marked using anti-CD45 bound to a different fluorochrome. The sample was then pooled and most of it was enriched for cells binding a tetramer containing a segment of the T antigen derived from Merkel cell polyomavirus. (FIG. created in BioRender) (5B) Gating for T antigen-binding B cells in the combined samples and (5C) CD45 gating to de-convolute the three samples in each pool. From this, a few hundred cells were sequenced and paired heavy and light chain sequences were obtained from 90 cells. From this, 5 monoclonal antibodies (mAbs) were initially produced from healthy control PBMCs and 5 mAbs from patient PBMCs. (5D) Binding of those 10 mAbs to the T antigen using Bio-Layer interferometry (BLI). Within these groups, three mAbs (1G04, 2H04 and 1 B09) from patients bound well, with the other two from patients binding less strongly. Three mAbs from control samples bound moderately well, with the other two failing to bind in this assessment.

FIGS. 6A-6E. Analysis of T-Ag-specific B cells using 10× single cell sequencing. (6A) % of CD19⁺ B cells in MCC skin tumors, MCC tumor-infiltrated lymph nodes, and healthy control PBMC. Mann-Whitney test determined p value. (6B) Analysis of B cells binding T-Ag APC and T-Ag PE-Oligo tetramers by flow cytometry. (6C) Analysis of Oligo tetramer reads following single cell sequencing. The gated cells are considered T-Ag-specific in downstream analyses. (6D) % of B cells that were T-Ag-specific in MCC skin tumors, MCC tumor-infiltrated lymph nodes, and healthy control PBMC. (6E) Assessment of T-Ag binding of 8 antibodies from T-Ag Oligo tetramer+B cells gate and 7 antibodies outside of that gate from 6C. The antibodies that bound T-Ag by BLI are indicated by a star and the non-binding antibodies indicated by a triangle and are highlighted in 6D.

FIGS. 7A-7E. The presence of T-Ag specific germinal center B cells predicts positive MCC outcome. (7A) UMAP of B cells from combined skin tumor and tumor-infiltrated lymph node samples. The cells shown in color are T-Ag-specific and the different B cell subtypes displayed in different colors. (7B) Expression of select genes and CD71 protein by the indicated B cell subsets. (7C) Frequency of T-Ag-specific cells of each listed subtype in tumor samples compared to B cells of unknown specificity for each patient. The numbers next to the bars are the number of cells assessed. (7D) Kaplan-Meier curves showing the amount of time that elapsed before MCC progression after tumor analysis stratified by whether germinal center B cells were detected with in the T-Ag-specific (top) or unknown specificity cells (bottom) in tumor-infiltrated lymph node samples from immunocompromised patients. (7E) Details about the cohort assessed. The two samples in 7C and 7E (marked with a star) were obtained from immunocompromised patients and were excluded from analysis in 7D.

FIGS. 8A, 8B. Phenotype of T-Ag-specific B cells in PBMC. (8A) Detection of mutated antibody heavy chain (IgH) nucleotides in unknown specificity and T-Ag-specific B cells from three MCC PBMC samples. (8B) Frequency of unknown specificity and T-Ag-specific B cells of the listed subtype in three MCC PBMC samples.

FIGS. 9A-9C. Detection of T-Ag-specific CD4+ T cells. (9A) Identification of CD154⁺CD69⁺CD137⁺CD4⁺ T cells in MCC patient PBMC following 18 hour incubation with common T-Ag₁₋₁₃ peptide or DMSO. (9B) Lentiviral-mediated expression of a TCR identified in 9A in autologous CD4⁺ T cells detected using an antibody specific for a portion of the murine TCRβ constant region included in the transduced TCR versus fluorescence minus one (FMO) control. (9C) Proliferation in cultures of T-Ag-specific TCR-transduced CD4⁺ T cells mixed with autologous irradiated PBMCs used as antigen-presenting cells incubated with T-Ag₁₋₁₃ peptide, phytohemagglutinin (PHA) positive control or DMSO negative control.

FIG. 10. The presence of T_{FH} cells in MCC tumor samples. Expression of CXCL13 and TCF7 in CD4⁺ T cells from 10 combined MCC tumor samples.

FIG. 11. Examining cell localization in an MCC tumor. Detection of select markers on a 4-micron MCC tumor section using 41-marker multiplexed immunohistochemistry. Not shown AE1/AE3, Cathepsin, CD1c, CD3e, CD11b, CD11c, CD15, CD27, CD34, CD45, CD45RO, CD66b, CD68, CD79b, CD123, CD141, CD163, CLEC9a, CXCR1, CXCR5, E-cadherin, Foxp3, HLA-DR, LAG3, LOX1, HLA class I, PD-1, PD-L1, S100A8, TIGIT, Tim3, TOX, TCF1/7, and VISTA.

FIGS. 12A-12D. The SLAP MCC Model. (12A) Approach to generate SLAP MCC mouse model. (12B) Gross tumors arising in the SLAP mice histologically resemble human MCC tumors and express truncated large T-Ag assessed using immunohistochemistry. (12C) Detection of serum antibodies specific for large and small T-Ags in SLAP mice after tumor detection. Data displayed as the combined area under the curve (AUC) of serum antibody binding the two T-Ags. (12D) Reduction in serum reactivity to large and small T-Ags when samples were pre-incubated with common T-Ag prior to assessment. A common T-Ag-specific monoclonal antibody (mAb) was used to demonstrate maximal blockade.

FIGS. 13A, 13B. Growth of SLAP tumor lines in C57BL/6 recipients. (13A) Mouse MCC SLAP cell lines grow in vitro as suspension clusters and express large T-Ag, ATOH1 and SOX2 by immunoblotting. (13B) Detection of T-Ag, Ki67, ISL1, SOX2, KRT8, and SYN expression by SLAP tumors in C57BL/6 recipients four weeks after subcutaneous injection of 10⁶ SLAP cells using immunohistochemistry or immunofluorescence.

FIGS. 14A-14D. Potent Immune response stimulated by T-Ag vaccination can slow growth of T-Ag-expressing melanoma. (14A) Detection of T-Ag-specific serum antibodies 28 days after subcutaneous injection of 10 μg self-amplifying T-Ag or control mRNA vaccine at day 0 and 14. Data displayed as the combined area under the curve (AUC) of antibody binding to large and small T-Ag. (14B) CD8⁺ T cell responses to H-2k^{b}-binding peptide common T-Ag₁₉₋₂₇ and CD4+ T cell responses to I-A^{b}-binding peptide large T-Ag₁₃₆₀₋₁₆₀ & ₁₄₁₋₁₆₀ measured by IFN{ELISpot 28 days after mRNA vaccination. Displayed as the number of spots per million splenocytes. (14C) Survival of mice following intradermal injection of 1.5×10⁵ B16^{T-Ag} tumor cells in mice previously vaccinated with T-Ag or control mRNA vaccine (n=10). (14D) Survival of mice following subcutaneous injection of 2×10⁵ B16^{T-Ag} tumor cells following four weekly treatments with 40 μg of T-Ag^{S220A} or control DNA vaccine via intradermal injection followed by electroporation (n=10).

FIG. 15. Sequences supporting the disclosure including: Human Intronic Region 1 to Target for Genetic Construct Insertion (SEQ ID NO: 1); Human Intronic Region 2 to Target for Genetic Construct Insertion (SEQ ID NO: 2); CD3ζ signaling domain-A (SEQ ID NO: 3); CD3ζ signaling domain-B (SEQ ID NO: 4); CD3ζ signaling domain-C (SEQ ID NO: 5); CD3zeta signaling domain encoding sequence-A (SEQ ID NO: 6); CD3ζ signaling domain encoding sequence-B (SEQ ID NO: 7); 4-1BB costimulatory domain-A (SEQ ID NO: 8); 4-1BB costimulatory domain-B (SEQ ID NO: 9); 4-1BB costimulatory domain-C(SEQ ID NO: 10); 4-1BB signaling encoding sequence-A (SEQ ID NO: 11); 4-1BB signaling encoding sequence-B (SEQ ID NO: 12); 4-1BB signaling coding sequence-C(SEQ ID NO: 13); CD28 Transmembrane Domain-A (SEQ ID NO: 14); CD28 Transmembrane Domain—B (SEQ ID NO: 15); CD28 Transmembrane Domain—C (SEQ ID NO: 16); CD28TM encoding sequence-A (SEQ ID NO: 17); CD28TM encoding sequence-B (SEQ ID NO: 18); CD28TM encoding sequence-C(SEQ ID NO: 19); CD28TM encoding sequence-D (SEQ ID NO: 20); IgG4 hinge-A (SEQ ID NO: 21); IgG4 hinge coding sequence-A (SEQ ID NO: 22); IgG4 hinge coding sequence-B (SEQ ID NO: 23); IgG4 hinge coding sequence-C(SEQ ID NO: 24); IgG4-CH2 domain-A (SEQ ID NO: 25); IgG4-CH2 domain-B (SEQ ID NO: 26); IgG4-CH2 domain encoding sequence-A (SEQ ID NO: 27); IgG4-CH2 domain encoding sequence-B (SEQ ID NO: 28); IgG4-CH3 domain-A (SEQ ID NO: 29); IgG4 CH3 domain-B (SEQ ID NO: 30); IgG4-CH3 encoding sequence-A (SEQ ID NO: 31); IgG4 CH3 domain coding sequence-B (SEQ ID NO: 32); Intermediate Spacer (IgG4 hinge-CH3 domain) (SEQ ID NO: 33); Intermediate Spacer encoding sequence (IgG4 hinge-CH3 domain) (SEQ ID NO: 34); Long Spacer (IgG4 hinge-CH2 domain-CH3 domain) (SEQ ID NO: 35); Long Spacer encoding sequence (IgG4 hinge-CH2 domain-CH3 domain) (SEQ ID NO: 36); Thoseaasigna Virus 2A (T2A) Peptide-v1 (SEQ ID NO: 37); Thoseaasigna Virus 2A (T2A) Peptide-v2 (SEQ ID NO: 38); Porcine Teschovirus-1 2a (P2A) Peptide (SEQ ID NO: 39); Equine Rhinitis A Virus (ERAV) 2A (E2A) Peptide (SEQ ID NO: 40); Foot-And-Mouth Disease Virus 2A (F2A) Peptide (SEQ ID NO: 41); T2A encoding sequence-v1 (SEQ ID NO: 42); and T2A encoding sequence-v2 (SEQ ID NO: 43).

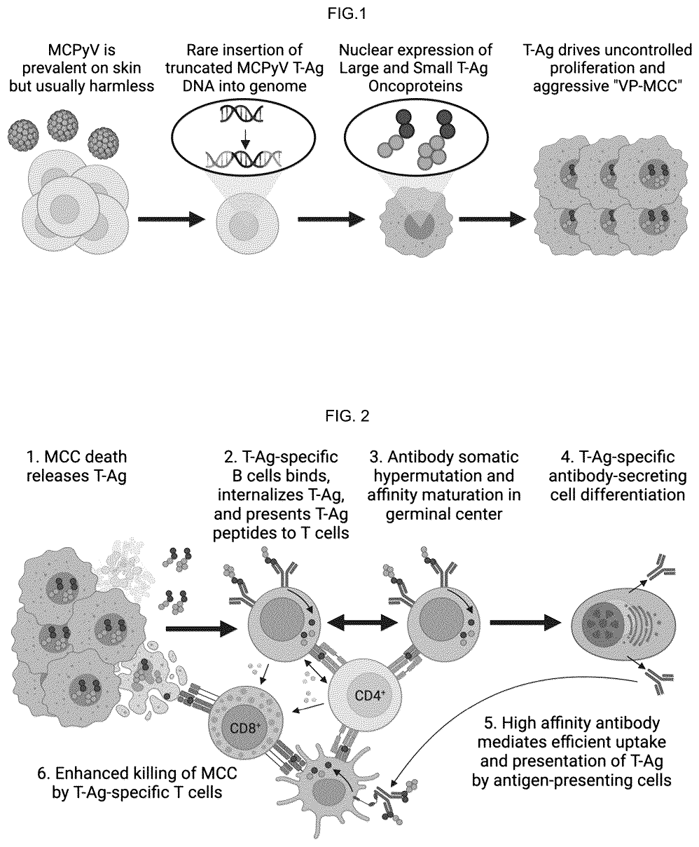

### DETAILED DESCRIPTION

Merkel cell carcinoma (MCC) is a rare, aggressive skin cancer with a reported incidence of 2,000 new cases each year in the United States. MCC refers to hyperproliferative or uncontrolled growth of cells in the skin that share some characteristics with normal Merkel cells of the skin, which may be infected with a Merkel cell polyomavirus (MCPyV) or have a high somatic mutation burden (e.g., due to exposure to UV light) in one or more genes including RB1, TP53, chromatin modification pathway genes (e.g., ASXL1, MLL2, MLL3), INK pathway genes (e.g., MAP3K1, TRAF7), and DNA-damage pathway (e.g., ATM, MSH2, BRCA1). The MCC arising from infection with MCPyV may also be referred to as “MCPyV-positive MCC” and MCC arising from a high somatic mutation burden may also be referred to as “MCPyV-negative MCC.”

The number of people diagnosed with MCC has been rising quickly over the past few decades. An increased risk of MCC has been linked with immunosuppression related to UV radiation, viral infections, organ transplantation, and chronic lymphocytic leukemia (Paulson et al., J. Invest. Dermatol. 729:1547, 2009; Goh et al., Oncotarget 7:3403, 2016; Feng et al., Science 379: 1096, 2008). While MCC is more frequently observed in immunocompromised or elderly populations, more than 90% of patients with MCC do not appear to be observably immune compromised (Heath et al., J. Am. Acad. Dermatol. 58: 375, 2008). Nonetheless, MCC is more lethal than melanoma with a reported 40% mortality rate (Heath et al., 2008). Once metastasized, MCC has an even worse prognosis. Only 18% of patients having stage IV metastatic disease survive for 5 years following diagnosis (Lemos and Nghiem, J Invest Dermatol. 127(9):2100-3, 2007).

Merkel cell polyomavirus (MCPyV) is associated with 80% of MCCs. MCPyV is a naked double-stranded DNA virus belonging to the Polyomaviridae family. The early region of its circular genome includes the “Tumor” (T) antigen gene locus which encodes the large T (LT) and small T (sT) antigens. LT and sT are products of alternate reading frames of the LT open reading frame (ALTO) (Pietropaolo et al., Cancers (Basel) 2020, 12(7):1774) and share 78 amino acids at the amino-terminus (the common T antigen) and their expression appears to be necessary for MCC maintenance (Houben et al., J. Virol. 84: 7064, 2010).

The present disclosure describes antibodies and binding fragments thereof that bind the common tumor (T) antigen of Merkel cell polyomavirus (also referred to herein as MCPyV T antigen, T antigen, or T-Ag).

Principles of antigen processing by antigen presenting cells (APC) (such as dendritic cells, macrophages, lymphocytes or other cell types), and of antigen presentation by APC to T cells, including major histocompatibility complex (MHC)-restricted presentation between immunocompatible (e.g., sharing at least one allelic form of an MHC gene that is relevant for antigen presentation) APC and T cells, are well established (see, e.g., Murphy, Janeway's Immunobiology (8^{th} Ed) 2011 Garland Science, NY; chapters 6, 9 and 16). “Major histocompatibility complex” (MHC) refers to glycoproteins that deliver peptide antigens to a cell surface. MHC class I molecules are heterodimers having a membrane spanning a chain (with three a domains) and a non-covalently associated b2 microglobulin. MHC class II molecules are composed of two transmembrane glycoproteins, a and b, both of which span the membrane. Each chain has two domains. MHC class I molecules deliver peptides originating in the cytosol to the cell surface, where a peptide:MHC complex is recognized by CD8⁺ T cells. MHC class II molecules deliver peptides originating in the vesicular system to the cell surface, where they are recognized by CD4⁺ T cells. Human MHC is referred to as human leukocyte antigen (HLA).

Processed antigen peptides originating in the cytosol (e.g., tumor antigen or intracellular pathogen) are generally from 7 amino acids to 11 amino acids in length and will associate with class I MHC molecules, whereas peptides processed in the vesicular system (e.g., bacterial or viral) will generally vary in length from 10 amino acids to 25 amino acids and associate with class II MHC molecules.

In certain examples, the T antigen of Merkel cell polyomavirus refers to a naturally or synthetically produced portion of a Merkel cell polyomavirus common T antigen ranging in length from 7 amino acids to 15 amino acids, which can form a complex with an MHC (e.g., HLA) molecule.

An antibody or binding fragment thereof within the context of the currently disclosed antibodies refers to a protein or polypeptide that specifically binds to a Merkel cell polyomavirus T antigen epitope. The T antigen epitope can be on a T antigen peptide or fragment thereof. In some embodiments, a protein or polypeptide specifically binds to a molecule including the MCPyV T antigen epitope, such as a MCPyV T antigen peptide complexed with an MHC or an HLA molecule, e.g., on an immune cell surface, with at or at least about an avidity or affinity sufficient to elicit an immune response.

The disclosed antibodies can be used in in cell-based immunotherapies, antibody-based therapies, and detection assays in the context of infection with Merkel cell polyomavirus and the associated Merkel cell carcinoma.

In particular embodiments, cell-based immunotherapies include B cells genetically modified to express an antibody disclosed herein. B cell activity in the tumor microenvironment has been shown to be correlated with positive clinical outcomes in the treatment of cancer.

Embodiments disclosed herein also include cell-based therapies including other immune cells genetically modified to express a recombinant receptor, such as a chimeric antigen receptor (CAR) or an engineered T cell receptor (eTCR) having a binding domain of an antibody disclosed herein.

Antibody-based therapies can include, for example, “free” antibodies or antibody conjugates among numerous other therapeutic and diagnostic formats disclosed elsewhere herein. For example, antibody conjugates can include an antibody or binding fragment thereof as a binding domain linked to an immunotoxin, drug, detectable label, and/or radioisotope.

In particular embodiments, detection assays include antibodies or binding fragments thereof used in a platform for detecting the presence of the T antigen in a sample. In particular embodiments, the antibodies or binding fragments thereof are immobilized on a solid support to form, for example, immunobeads, immunoassays, and lateral flow assays. In certain examples, antibodies or binding fragments thereof are used to provide a control level in an assay.

The current disclosure also describes a link between the phenotype of T-Ag-specific B cells in tumor-infiltrated lymph nodes and disease outcome. When T-Ag-specific germinal center B cells are detected in lymph node samples, the patient usually remains MCC-free for a significant period of time (years). If T-Ag-specific germinal B cells are not detected in lymph node samples, then the patient should be administered an additional therapy for MCC because it is likely to progress.

Aspects of the current disclosure are now described with additional detail and options as follows: (i) Antibodies; (ii) B Cells; (iii) Other Immune Cells; (iv) Recombinant Receptors; (v) Control Features for Recombinantly-Expressed Molecules in Cell-Based Therapies; (vi) Cell Sample Collection and Cell Enrichment; (vii) Genetically-Modifying Cell Populations; (viii) Cell-Based Formulations; (ix) Multi-Specific Binding Domain Molecules; (x) Antibody Conjugates; (xi) Linkers for Recombinantly-Expressed Molecules; (xii) Multimerization Domains for Recombinantly-Expressed Molecules; (xiii) Antibody Compositions for Administration; (xiv) Methods of Use—Treatment; (xv) Methods of Use—In Vivo Detection; (xvi) Ex Vivo Detection Assays and Methods; (xvii) Reference Levels and Experimental Controls; (xviii) Exemplary Embodiments; (xix) Experimental Examples; and (xx) Closing Paragraphs. These headings are provided for organizational purposes only and do not limit the scope or interpretation of the disclosure.

(i) Antibodies. Naturally occurring antibodies are proteins produced by B cells of the immune system that recognize antigens on foreign or diseased objects such as pathogenic bacteria, viruses, fungi, cancer cells, and/or tumors. If an antibody recognizes an antigen expressed by a foreign or diseased object, it can elicit an immune response against the foreign or diseased object.

Upon exposure to an antigen on a foreign or diseased object, B cells can be activated to produce protective antibodies. Antibodies can either be secreted into the blood by the B cells or can be membrane bound (known as B cell receptors or BCR).

As indicted, antibodies bind epitopes on antigens. The term antigen refers to a molecule or a portion of a molecule capable of being bound by an antibody. An epitope is a region of an antigen that is bound by the variable region of an antibody. Epitope determinants can include chemically active surface groupings of molecules such as amino acids, sugar side chains, phosphoryl or sulfonyl groups, and can have specific three-dimensional structural characteristics, and/or specific charge characteristics. When the antigen is a protein or peptide, the epitope includes specific amino acids within that protein or peptide that contact the variable region of an antibody.

The present disclosure provides antibodies that bind the common T antigen of Merkel cell polyomavirus. In particular embodiments, the common T antigen includes the 78 amino acids at the amino-terminus of the large T antigen and small T antigen. In particular embodiments, the small T antigen from the Merkel cell polyomavirus (NCBI Ref: YP_009111422.1) includes the sequence:

(SEQ ID NO: 44)
MDLVLNRKEREALCKLLEIAPNCYGNIPLMKAAFKRSCLK
HHPDKGGNPVIMMELNTLWSKFQQNIHKLRSDFSMFDEVS
TKFPWEEYGTLKDYMQSGYNARFCRGPGCMLKQLRDSKCA
CISCKLSRQHCSLKTLKQKNCLTWGECFCYQCFILWFGFP
PTWESFDWWQKTLEETDYCLLHLHLF.

In particular embodiments, the large T antigen from the Merkel cell polyomavirus (NCBI Ref: YP: 009111421.1) includes the sequence:

(SEQ ID NO: 45)
MDLVLNRKEREALCKLLEIAPNCYGNIPLMKAAFKRSCLK
HHPDKGGNPVIMMELNTLWSKFQQNIHKLRSDFSMFDEVD
EAPIYGTTKFKEWWRSGGFSFGKAYEYGPNPHGTNSRSRK
PSSNASRGAPSGSSPPHSQSSSSGYGSFSASQASDSQSRG
PDIPPEHHEEPTSSSGSSSREETTNSGRESSTPNGTSVPR
NSSRTDGTWEDLFCDESLSSPEPPSSSEEPEEPPSSRSSP
RQPPSSSAEEASSSQFTDEEYRSSSFTTPKTPPPFSRKRK
FGGSRSSASSASSASFTSTPPKPKKNRETPVPTDFPIDLS
DYLSHAVYSNKTVSCFAIYTTSDKAIELYDKIEKFKVDFK
SRHACELGCILLFITLSKHRVSAIKNFCSTFCTISFLICK
GVNKMPEMYNNLCKPPYKLLQENKPLLNYEFQEKEKEASC
NWNLVAEFACEYELDDHFIILAHYLDFAKPFPCQKCENRS
RLKPHKAHEAHHSNAKLFYESKSQKTICQQAADTVLAKRR
LEMLEMTRTEMLCKKFKKHLERLRDLDTIDLLYYMGGVAW
YCCLFEEFEKKLQKIIQLLTENIPKYRNIWFKGPINSGKT
SFAAALIDLLEGKALNINCPSDKLPFELGCALDKFMVVFE
DVKGQNSLNKDLQPGQGINNLDNLRDHLDGAVAVSLEKKH
VNKKHQIFPPCIVTANDYFIPKTLIARFSYTLHFSPKANL
RDSLDQNMEIRKRRILQSGTTLLLCLIWCLPDTTFKPCLQ
EEIKNWKQILQSEISYGKFCQMIENVEAGQDPLLNILIEE
EGPEETEETQDSGTFSQ.

In particular embodiments, the common T antigen of Merkel cell polyomavirus includes the sequence:

(SEQ ID NO: 46)
MDLVLNRKEREALCKLLEIAPNCYGNIPLMKAAFKRSCLK
HHPDKGGNPVIMMELNTLWSKFQQNIHKLRSDFSMFDE.

Naturally occurring/secreted antibody structural units include a tetramer. Each tetramer includes two pairs of polypeptide chains, each pair having one light chain and one heavy chain. The amino-terminal portion of each chain includes a variable region that is responsible for antigen recognition and epitope binding. The variable regions exhibit the same general structure of relatively conserved framework regions (FR) joined by three hyper variable regions, also called complementarity determining regions (CDRs). The CDRs from the two chains of each pair are aligned by the framework regions, which enables binding to a specific epitope. From N-terminal to C-terminal, both light and heavy chain variable regions include the domains FR1, CDR1, FR2, CDR2, FR3, CDR3 and FR4.

The assignment of amino acids to each domain can be in accordance with Kabat numbering (Kabat et al., (1991), “Sequences of Proteins of Immunological Interest,” 5th Ed. Public Health Service, National Institutes of Health, Bethesda, Md. (“Kabat” numbering scheme)); Chothia (Al-Lazikani et al., (1997) JMB 273, 927-948 (“Chothia” numbering scheme)), Martin (Abinandan et al., Mol Immunol. 45:3832-3839 (2008), “Analysis and improvements to Kabat and structurally correct numbering of antibody variable domains”), Gelfand, Contact (MacCallum et al., J. Mol. Biol. 262:732-745 (1996), “Antibody-antigen interactions: Contact analysis and binding site topography,” J. Mol. Biol. 262, 732-745.” (Contact numbering scheme)), IMGT (Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme)), AHo (Honegger A and Plückthun A, “Yet another numbering scheme for immunoglobulin variable domains: an automatic modeling and analysis tool,” J Mol Biol, 2001 Jun. 8; 309(3):657-70, (AHo numbering scheme)), North (North et al., J Mol Biol. 406(2):228-256 (2011), “A new clustering of antibody CDR loop conformations”), or other numbering schemes. Software programs, such as ABodyBuilder can also be used.

In particular embodiments, a binding domain that binds T antigen of the Merkel cell polyomavirus includes the 1C10 antibody. In particular embodiments, the 1C10 antibody includes a variable heavy chain including the sequence:

(SEQ ID NO: 47)
EVQLLESGAEVKKPGASVRLSCKASGYTFTIYYMHWVRQA
PGHGLEWMGIINPSGGRATYAEKLQGRLTMTRDTSTNTVY
LELSSLRSEDTAVYYCARDRGGQNCTATRCPHYFFSGMDV
WGQGTTVTVSS

and a variable light chain sequence including the sequence:

(SEQ ID NO: 48)
QYVLTQPPSVSVSPGQTARITCSGDELSKQYAYWYQQKPGQAPVLMIYKD
TERPSGIPERFSGSSSGTTVTLSIIGVQAEDEADYYCQSADTTVFFGGGT
RLTVL.

In particular embodiments, a binding domain that binds T antigen of the Merkel cell polyomavirus includes the 3B09 antibody. In particular embodiments, the 3B09 antibody includes a variable heavy chain including the sequence:

(SEQ ID NO: 49)
QLQLQESGPGLVKPSETLSLTCAVSVGSISSSSYYWGWIRQPPGKGLEWI
GSISHSGNTYYNPSLKSRLTISVDTSKNQISLKVTSVTAADTAVYYCARQ
PQYFYDSSGHSLAYYGLDVWGRGTTVTVSS

and a variable light chain sequence including the sequence:

(SEQ ID NO: 50)
QFVLTQPASVSGSPGQSITISCTGASSDFGGYNSVSWYQQHSGKAPKLMI
YDVHNRPSGVSNRFSGSKSGNTASLTISGLQADDEADYYCSSYTTSTFWV
FGGGTKLTVL.

In particular embodiments, a binding domain that binds T antigen of the Merkel cell polyomavirus includes the 1 B09 antibody. In particular embodiments, the 1B09 antibody includes a variable heavy chain including the sequence:

(SEQ ID NO: 51)
EVOLVESGGGVVQPGGSLRLSCAASGFNFSNYAMHWVRQAPGMGLEWVAV
LSYDGSNEYYAGSVRGRFTISRDNSKNTLYLQIHSLRVEDTAVFYCARPR
GSARRRFFYFDHWGQGALVTVSS

and a variable light chain sequence including the sequence:

(SEQ ID NO: 52)
QSVLTQPASVSGSPGQSITISCTGTINDVGGFDLVSWYQQLPGKAPKVLI
YDVSDRPSGVSSRFSGSKSGNTASLTISGLQAEDEADYYCSSYTTSTSLL
YVFGTGTKVTVL.

In particular embodiments, a binding domain that binds T antigen of the Merkel cell polyomavirus includes the 1G04 antibody. In particular embodiments, the 1G04 antibody includes a variable heavy chain including the sequence:

(SEQ ID NO: 53)
QVQLQESGAGLVRPSQTLSLTCAVSGDSISGGVYSWSWIRQTPEKGLEWI
GYIDQSGSTYYSPSLKSRVTISVDTSENQFSLKLRSVTAADTAVYFCARA
SPMMVVLNWGQGTRVTVSS

and a variable light chain sequence including the sequence:

(SEQ ID NO: 54)
DIQMTQSPSTLSASVGDRVTITCRASQSVDTWLAWYQQKPGKAPKLLIHL
ASNLDSGVPTRFSGSGSGTEFTLTISSLQPDDFATYYCQQYHTHSGTFGQ
GTKVEIK.

In particular embodiments, a binding domain that binds T antigen of the Merkel cell polyomavirus includes the 2H04 antibody. In particular embodiments, the 2H04 antibody includes a variable heavy chain including the sequence:

(SEQ ID NO: 55)
EVQVVESGGGLVKPGGSLRLSCAGTGFTFNVYAMNWVRQAPGKGLEWVSS
ISPGGTSTYYADSVKGRFTISRDDAKNSLDLQMNSLRVEDTAIYYCARDS
IFGVIMGGLDPWGQGTLVTVSS

and a variable light chain sequence including the sequence:

(SEQ ID NO: 56)
SYVLTQPPSVSVAPGQTASFTCGVRNHITDGVHWYLQKPGQAPVLVVYDD
RDRPSGIPERFSGSRSRNTATLTISRVEAGDEADYYCQVWDRNRDQVIFG
GGTKLTVL.

In particular embodiments, a binding domain that binds T antigen of the Merkel cell polyomavirus includes the W056-03 antibody. In particular embodiments, the W056-03 antibody includes a variable heavy chain including the sequence:

(SEQ ID NO: 169)
GVQLVESGGGFVQPGGSLRLSCAASGVTFSRFDMNWVRQAPGKGLEWVSY
ISSSGTTIYYADSVKGRFTISRDNAKNSLYLRLNSLRADDTAVYYCARDD
YTGWYYFDSWGQGTLVTVSS

and a variable light chain sequence including the sequence:

(SEQ ID NO: 170)
QSVLTQPPSVSGAPGQRVTISCTGSSSNIGAGFDVHWYQQFPGTAPKLLI
YGNSNRPSGVPDRFSGSKSGTSASLAITGLQAEDEADYYCQSYDSSLYVF
GTGTKVTVL.

In particular embodiments, the W056-03 antibody includes a lambda light chain.

In particular embodiments, a binding domain that binds T antigen of the Merkel cell polyomavirus includes the W056-04 antibody. In particular embodiments, the W056-04 antibody includes a variable heavy chain including the sequence:

(SEQ ID NO: 171)
QVTLRESGPALVKPTQTLTLTCTFSGFSLSTSGMCVSWIRQPPGKALEWL
ARIDWDDDKYYNTSLKTRLTISKVTSKNRVVLTMTNMDPVDTATYYCARQ
ELVGAWFDPWGQGTLVTVSS

and a variable light chain sequence including the sequence:

(SEQ ID NO: 172)
RFSGSKSGNTASLTISGLQAEDEAEYYCISYTSSSISCVFGTGTKVTVLQ
SALTQPASVSGSPGQSITVSCTGTSGDVGGYNYVSWYQQHPGKAPKLIIY
EVSNRPSGVSN.

In particular embodiments, the W056-04 antibody includes a lambda light chain.

In particular embodiments, a binding domain that binds T antigen of the Merkel cell polyomavirus includes the W056-05 antibody. In particular embodiments, the W056-05 antibody includes a variable heavy chain including the sequence:

(SEQ ID NO: 173)
QVQLVESGGGVVQPGRSLRLSCAASGFTFSSYGMHWVRQAPGKGLEWVAV
ISYDGSTKYYADSVKGRFTISRDNSKNTLYLQMNSLRAADTAVYYCAKDG
TAVLVDRLDYWGQGTLVTVSS

and a variable light chain sequence including the sequence:

(SEQ ID NO: 174)
AIQLTQSPSSLSASVGDRVTITCRASQGISSYLAWYQQNPGKAPKLLIYA
ASTLQSGVPSRFSGSGSGTDFTLTISSLQPEDFATYYCQQLNSYPLTFGG
GTKVEIK.

In particular embodiments, the W056-05 antibody includes a kappa light chain.

In particular embodiments, a binding domain that binds T antigen of the Merkel cell polyomavirus includes the W056-06 antibody. In particular embodiments, the W056-06 antibody includes a variable heavy chain including the sequence:

(SEQ ID NO: 175)
EVQLVQSGAEMKKPGESLRISCAASGYDFDTYWIAWVRQMPGKGLDWMGV
IYPDDSDTKYSPSFQGQVTISVDKSINTAYLQWTSLKASDTAIYYCARAT
GDYWGQGTLVTVSS

and a variable light chain sequence including the sequence:

(SEQ ID NO: 176)
DIQMTQSPSALSASVGDRVTISCRASQNVSTWLTWYQQKPGAAPKRLIYE
ASTLQSGVPSRFSGRGSGTEFFLTISSLQPDDFATYFCQQYRSYSRTFGQ
GTKVEIR.

In particular embodiments, the W056-06 antibody includes a kappa light chain.

In particular embodiments, a binding domain that binds T antigen of the Merkel cell polyomavirus includes the W056-08 antibody. In particular embodiments, the W056-08 antibody includes a variable heavy chain including the sequence:

(SEQ ID NO: 177)
QVTLRESGPALVKPTQTLTLTCTFSGFSLSTSGMCVSWIRQPPGKALEWL
ARIDWDDDKYYNTSLKTRLTISKDTSKNRVVLTMTNMDPVDTATYYCARQ
ELVGAWFDPWGQGTLVTVSS

and a variable light chain sequence including the sequence:

(SEQ ID NO: 178)
QSALTQPASVSGSPGQSITVSCTGTSGDVGGYNYVSWYQQHPGKAPKLII
YEVSNRPSGVSNRFSGSKSGNTASLTISGLQAEDEAEYYCISYTSSSISC
VFGTGTKVTVL.

In particular embodiments, the W056-08 antibody includes a lambda light chain.

In particular embodiments, a binding domain that binds T antigen of the Merkel cell polyomavirus includes the W056-09 antibody. In particular embodiments, the W056-09 antibody includes a variable heavy chain including the sequence:

(SEQ ID NO: 179)
EVQLVQSGPEAKKPGESLRISCAGSGYDFNTYWIAWVRQMPGKGLDWMGV
IYPDDSDTKYSPSFQGQVTISVDKSITTAYLQWSSLKASDTAMYYCARAT
GDYWGQGTLVTVSS

and a variable light chain sequence including the sequence:

(SEQ ID NO: 180)
DIQMTQSPSALSASVGDRVTISCRASQNVSRWLTWYQQKPGAAPKRLIYE
ASTLQSGVPSRFSGRGSGTEFFLTISSLQPDDFATYFCQQYRSYSRTFGQ
GTKVEIR.

In particular embodiments, the W056-09 antibody includes a kappa light chain.

In particular embodiments, a binding domain that binds T antigen of the Merkel cell polyomavirus includes the W056-10 antibody. In particular embodiments, the W056-10 antibody includes a variable heavy chain including the sequence:

(SEQ ID NO: 181)
QVQLVQSGAEVKRPGSSVKVSCKASGATFSSYAISWVRQAPGQGLEWLGR
VTPIIDTTNYAQKFQGRVTITADESTSTAYMELSGLRSEDTAIYYCATRG
TYYDFWPSMDVWGQGTTVTVSS

and a variable light chain sequence including the sequence:
In particular

(SEQ ID NO: 182)
DIQMTQSPSSLSASVGDRVTITCRASQDIRNYLAWFQQKPGKAPKSLIYA
ASSLHSGVPSKFSGSGSGTDFTLTISGLQPEDFATYYCQQYNSYPLTFGG
GTRVEIK.

embodiments, the W056-10 antibody includes a kappa light chain.

In particular embodiments, a binding domain that binds T antigen of the Merkel cell polyomavirus includes the Z156-02 antibody. In particular embodiments, the Z156-02 antibody includes a variable heavy chain including the sequence:

(SEQ ID NO: 183)
QVQLQQWGAGLLKPSETLSLTCAVYGGSFSGYYWSWIRQPPGKGLEWIGE
INHSGSTNYNPSLKSRVTISVDTSKNQFSLKLSSVTAADTAVYYCASMSG
SYYGAFDYWGQGTLVTVSS

and a variable light chain sequence including the sequence:

(SEQ ID NO: 184)
SYELTQPPSVSVSPGQTARITCSGDALPKQYAYWYQQKPGQAPVLVIYKD
SERPSGIPERFSGSSSGTTVTLTISGVQAEDEADYYCQSADSSGTYYVFG
TGTKVTVL.

In particular embodiments, the Z156-02 antibody includes a lambda light chain.

In particular embodiments, a binding domain that binds T antigen of the Merkel cell polyomavirus includes the Z156-03 antibody. In particular embodiments, the Z156-03 antibody includes a variable heavy chain including the sequence:

(SEQ ID NO: 185)
QLQLQESGPGLVKPSETLSLTCTVSGGSISSSSYYWGWIRQPPGKGLEWI
GSIYYSGSTYYNPSLKSRVTISVDTSKNQFSLKLSSVTAADTAVYYCASI
IAALFDYWGQGTLVTVSS

and a variable light chain sequence including the sequence:

(SEQ ID NO: 186)
YELTQPSSVSVSPGQTARITCSGDVLAKKYARWFQQKPGQAPVLVIYKDS
ERPSGIPERFSGSSSGTTVTLTISGAQVEDEADYYCYSAADNNWVFGGGT
KLTVL.

In particular embodiments, the Z156-03 antibody includes a lambda light chain.

In particular embodiments, a binding domain that binds T antigen of the Merkel cell polyomavirus includes the Z249-01 antibody. In particular embodiments, the Z249-01 antibody includes a variable heavy chain including the sequence:

(SEQ ID NO: 187)
QVQLVKSGGGVVQPERSLRLSCAASGFTFKHYSMHWVRQAPGKGLEWVAH
ISYDGSKTNYADSVKGRFSISRDNSENTLYLQMNSLKTDDTALYYCVRDR
TTGHDFGFDYWGPGTQVIVSS

and a variable light chain sequence including the sequence:

(SEQ ID NO: 188)
HELTQPPSVSVSPGLTARITCSGDALSKKYAYWYQQKSGQAPVLVMFKDN
ERPAGVPERFSGSSSGTTVTLTISGVQAEDEADYYCQSTDSDTSYVIFGG
GTKLTVL.

In particular embodiments, the Z249-01 antibody includes a lambda light chain.

In particular embodiments, a binding domain that binds T antigen of the Merkel cell polyomavirus includes the Z249-02 antibody. In particular embodiments, the Z249-02 antibody includes a variable heavy chain including the sequence:

(SEQ ID NO: 189)
QVQLVQSGAEVKKPGSSLKVSCKTSGGFFSSYRINWVRQAPGQGLEWMGG
LVPIFETAKYAQKFQGRVSITADESTSTVYMEVTSLTSDDTAVYYCARGS
GSGLTYFQTWGQGSLVTVSS

and a variable light chain sequence including the sequence:
In particular embodiments, the Z249-02 antibody includes a kappa light chain.

(SEQ ID NO: 190)
EIVLTQSPGTLSLSPGERATLSCRASQSITTYYLAWYQQKPGQAPRLLIY
GASSRATGIPDRFSGSGSGTDFTLTISRLDPEDFAVYYCQQYDSSPTFGQ
GTKVEVK.

In particular embodiments, a binding domain that binds T antigen of the Merkel cell polyomavirus includes the Z249-03 antibody. In particular embodiments, the Z249-03 antibody includes a variable heavy chain including the sequence:

(SEQ ID NO: 191)
QLQLQESGPGLVKPSETLPLSCTVSGGSITSISHYWGWIRQPPGKGLEWI
GSMYYSGTTYYNPSLKSRVTMSVDTSKNQFSLKLSSVTAADTAVYYCARH
DVSRFDPWGQGTLVTVSS

and a variable light chain sequence including the sequence:

(SEQ ID NO: 192)
QSALTQPASVSGSPGQSITISCTGTSSDVGGYNYVSWYQHHPGKAPKLMI
YDVTNRPSGVSNRFSGSKSGNTASLTISGLQAEDEADYYCSSYTTSTWVF
GGGTKLTVL.

In particular embodiments, the Z249-03 antibody includes a lambda light chain.

In particular embodiments, a binding domain that binds T antigen of the Merkel cell polyomavirus includes the Z249-04 antibody. In particular embodiments, the Z249-04 antibody includes a variable heavy chain including the sequence:

(SEQ ID NO: 193)
RGQLVESGGGVVQPGRALRLSCVASGFTFSNFDMHWIRQAPGKGLEWVAV
ISYDESKRQHADSVKGRFTISRDKSKNTVYLQMNSLRAEDTAVYYCATES
TQYFGALDTWGQGTLVTVSS

and a variable light chain sequence including the sequence:

(SEQ ID NO: 194)
SYELTQPPSVSVSPGQTARITCSGDGLADQYVYWYRQRPGQAPALVMYKD
SERPSGIPERFSGASSGTQVTLTISGVQAEDEAEYFCQSTDRSGSYLFGG
GTTLTVL.

In particular embodiments, the Z249-04 antibody includes a lambda light chain.

In particular embodiments, a binding domain that binds T antigen of the Merkel cell polyomavirus includes the Z249-05 antibody. In particular embodiments, the Z249-05 antibody includes a variable heavy chain including the sequence:

(SEQ ID NO: 195)
QVQLAESGGGVVQPGRSLRLSCAASGFNFNFYTMHWVRQAPGKGLEWVAL
VFYDGSKTYYADSVKGRSTISRDNSRRTLFLEINALRPEDTAMYYCVRDR
TTGHDYGMDVWGQGTTVIVSS

and a variable light chain sequence including the sequence:

(SEQ ID NO: 196)
SYELTQPPSVSVSPGQTATIACSGAGLANLYTYWYQQKPGQAPILLIYKD
TERPSGIPERFSGSNSGTTVTLTISGVQAGDEADYYCQSADSSRSSVIFG
GGTKLTVL.

In particular embodiments, the Z249-05 antibody includes a lambda light chain.

In particular embodiments, a binding domain that binds T antigen of the Merkel cell polyomavirus includes the Z249-06 antibody. In particular embodiments, the Z249-06 antibody includes a variable heavy chain including the sequence:

(SEQ ID NO: 197)
QVQLVQSGAEVKKPGSSLKVSCKTSGGFFSSYRINWVRQAPGQGLEWMGG
LVPIFETAKYAQKFQGRVSITADESTSTVYMEVTSLTSDDTAVYYCARGS
GSGLTYFQTWGQGSLVTVSS

and a variable light chain sequence including the sequence:

(SEQ ID NO: 198)
EIVLTQSPGTLSLSPGERATLSCRASQSITTYYLAWYQQKPGQAPRLLIY
GASSRATGIPDRFSGSGSGTDFTLTISRLDPEDFAVYYCQQYDSSPTFGQ
GTKVEVK.

In particular embodiments, the Z249-06 antibody includes a kappa light chain.

In particular embodiments, a binding domain that binds T antigen of the Merkel cell polyomavirus includes the Z308-03 antibody. In particular embodiments, the Z308-03 antibody includes a variable heavy chain including the sequence:

(SEQ ID NO: 199)
QVTLRQSGPALVKSTQTLTLTCTFSGFSLSTGGVCINWIRQPPGKALEWL
ARIDWDNDKYYRPSLKTRLTISKDTSKNQVVLTMTNMDPVDTATYYCARM
TGLYSDNWKGFDIWGQGTMVTVSS

and a variable light chain sequence including the sequence:

(SEQ ID NO: 200)
EIVLTQSPGTLSLSPGERATLSCRASQSVDSTYLAWYQQKPGQAPRLLIY
SASRRATGIPDRFSGSGSGTDFTLTISRLEPEDFAFYYCQQYGSAPGYTF
GQGTKLEIK.

In particular embodiments, the Z308-03 antibody includes a kappa light chain.

In particular embodiments, a binding domain that binds T antigen of the Merkel cell polyomavirus includes the Z308-04 antibody. In particular embodiments, the Z308-04 antibody includes a variable heavy chain including the sequence:

(SEQ ID NO: 1309)
EVQLVQSGAEVKKPGESLKISCKVSGYRFTSYWIGWVRQMPGKGLEWMGV
IYPPDSDTAYSPSFQGQVTMSVDNSISTAYLQWSNLKASDTAMYYCARQG
RGFDYWGQGTLVTVSS

and a variable light chain sequence including the sequence:

(SEQ ID NO: 1310)
DIQVAQSPSSLSASVGDSVTITCRTSQNIYTYLNWYQQKPGKAPNLLINT
ASSLQGGVPSRFSGSGSGTDFTLTISSLRPEDFATYYCQQSYDSPRTFGQ
GTRVESK.

DIQVAQSPSSLSASVGDSVTITCRTSQNIYTYLNWYQQKPGKAPNLLINTASSLQGGVPSRFSG SGSGTDFTLTISSLRPEDFATYYCQQSYDSPRTFGQGTRVESK (SEQ ID NO: 1310. In particular embodiments, the Z308-04 antibody includes a kappa light chain.

In particular embodiments, a binding domain that binds T antigen of the Merkel cell polyomavirus includes the Z308-05 antibody. In particular embodiments, the Z308-05 antibody includes a variable heavy chain including the sequence:

(SEQ ID NO: 201)
QVTLRQSGPALVKPTQTLTLTCTVSGFSLNTAEVCWNWIRQPPGKALEWL
ARIDWDDDKYIRPSLKTRLTISKDTSKNQVVLTMTNMDPVDTATYYCARM
TGLYTDNWKGFDMWGQGTMVTVSS

and a variable light chain sequence including the sequence:

(SEQ ID NO: 202)
EIVLTQSPDTLSLSPGERATLSCRASQTIDSTYFAWYQQKPGQAPRLLIY
SASRRATGVPDRFSGSASGTDFTLTISRLEPEDFAFYYCQQYGHAPGYTF
GQGTKLDIK.

In particular embodiments, the Z308-05 antibody includes a kappa light chain.

In particular embodiments, a binding domain that binds T antigen of the Merkel cell polyomavirus includes the Z504-03 antibody. In particular embodiments, the Z504-03 antibody includes a variable heavy chain including the sequence:

(SEQ ID NO: 203)
QVQLVQSGAEVKKPGSSVKVSCKAPGGTFSRYAISWVRQAPGQGLEWMGG
IIPMFDITNYAQKLQDRIRITADESTSTAYMELSSLRSEDTALYYCATES
CSGGNCYWDHDAFDIWGQGTMVTVSS

and a variable light chain sequence including the sequence:
In particular embodiments, the Z504-03 antibody includes a kappa light chain.

(SEQ ID NO: 204)
EIVLTQSPGTLSLSPGERATLSCRASQSLSSSYLAWYQQKAGQAPRLLIY
GASTRATGIPDRFSGSGSGTDFTLTISRLEPEDFAVYYCQQYGRSLPRTF
GQGTKVEIK.

In particular embodiments, a binding domain that binds T antigen of the Merkel cell polyomavirus includes the Z504-04 antibody. In particular embodiments, the Z504-04 antibody includes a variable heavy chain including the sequence:

(SEQ ID NO: 205)
QVQLVQSGAEVKKPGSSVKVSCKASGGTFSSYAISWVRQAPGQGLEWMGG
IIPIFDIANYAQKFQGRVTITADESTSTAFMELSSLRSEDTAVYYCASEN
CGGGNCYWEHDAFDIWGQGTRVTVSS

and a variable light chain sequence including the sequence:

(SEQ ID NO: 206)
EIVLTQSPDTLSLSPGERATLSCRASQSVSGGDLAWYQQKPGQAPRLLMY
GASIRATGIPDRFSGSGSGTDFTLTISRLEPEDFVVYYCQHYGNSPPRTF
GQGTKVEIK.

In particular embodiments, the Z504-04 antibody includes a kappa light chain.

In particular embodiments, a binding domain that binds T antigen of the Merkel cell polyomavirus includes the Z504-06 antibody. In particular embodiments, the Z504-06 antibody includes a variable heavy chain including the sequence:

(SEQ ID NO: 1311)
QVQLVQSGAEVKKPGSSVKVSCKAYGGTFSTYAISWVRQAPGQGLEWMGG
IIPIFDIANYAQKFQGRVTITADESTSTAFMELSSLRSEDTAVYYCASEN
CSGGNCYWEHDAFDIWGQGTRVTVSS

and a variable light chain sequence including the sequence:

(SEQ ID NO: 1312)
EIVLTQSPDTLSLSPGERATLSCRASQSVSGGDLAWYQQKPGQAPRLLMY
GASIRATGIPDRFSGSGSGTDFTLTISRLEPEDFVVYYCQQYGNSPPRTF
GQGTKVEIK.

In particular embodiments, the Z504-06 antibody includes a kappa light chain.

In particular embodiments, a binding domain that binds T antigen of the Merkel cell polyomavirus includes the Z504-07 antibody. In particular embodiments, the Z504-07 antibody includes a variable heavy chain including the sequence:

(SEQ ID NO: 207)
EVQLVQSAAEVKKPGESLKISCQTSGYSFASFWIGWVRQM
PGKGLEWMGIIYPSDSDTTYSPTFQGLVTISVDKSSRTAY
LQWSSLKASDTAMYYCAKHGVGAAIDHWGQGTLVTVSS

and a variable light chain sequence including the sequence:

(SEQ ID NO: 208)
DIQMTQSPSSLSASVGDRVTITCRTSQVISGYLNWYQQRP
GKVPKLLIYTTSTLQPGVPSRFSGSGSGTDFTLSISNLQP
EDFATYYCQQSYSSPRTFGQGTKLEIK.

In particular embodiments, the Z504-07 antibody includes a kappa light chain.

In particular embodiments, a binding domain that binds T antigen of the Merkel cell polyomavirus includes the Z513-01 antibody. In particular embodiments, the Z513-01 antibody includes a variable heavy chain including the sequence:

(SEQ ID NO: 209)
QVQLQESGPGLVKPSQTLSLTCTVSGGSIGGGPYHWSWIR
QPAGKGLEWIGHVETSGDTKTNPSFRSRVTTSLDTATNQL
SLKVHSVTAADTAVYYCARGTAMDAFDIWGQGTVVIVSS

and a variable light chain sequence including the sequence:

(SEQ ID NO: 210)
DIQMTQSPSSVSASVGDRVTITCRASQDISNWLAWYQQRP
GKAPNLLIYVASGLQSGVPSRFSGSGSGTEFTLTISTLQP
EDSATYYCQQGNSFPLTFGGGTRVEIN.

In particular embodiments, the Z513-01 antibody includes a kappa light chain.

In particular embodiments, a binding domain that binds T antigen of the Merkel cell polyomavirus includes the Z513-02 antibody. In particular embodiments, the Z513-02 antibody includes a variable heavy chain including the sequence:

(SEQ ID NO: 211)
QVQLVESGGGVVQPGRSLRLSCAASGFSFSGYGMHWVRQA
PGKGLEWVAFIWYDGSSKKYGDSVKGRFIISRDNSKNTVY
LQMNSLRADDTAVYYCARDVLGELPDWGQGTLVTVSS

and a variable light chain sequence including the sequence:

(SEQ ID NO: 212)
DIQMTQSPSSLSASVGDRVTITCRASQGISNSLGWFQQKP
GKAPKSLIYAASSLQSGVPSKFSGSGSGTNFSLTISSLQP
EDFATYYCQQYNTYPLTFGGGTKVEIK.

In particular embodiments, the Z513-02 antibody includes a kappa light chain.

In particular embodiments, a binding domain that binds T antigen of the Merkel cell polyomavirus includes the Z538-03 antibody. In particular embodiments, the Z538-03 antibody includes a variable heavy chain including the sequence:

(SEQ ID NO: 213)
RVQLVESGGGVVQPGRSLRLSCATSGFTFSNYGLHWVRQA
PGKGLEWLAAIWFDGRTFYADSVKDRFSISRDNSKNTVYL
QINSLRVEDTAVYYCARETWSYAGGVFDYWGQGTLVIVSS

and a variable light chain sequence including the sequence:

(SEQ ID NO: 214)
QPVLTQSPSASAPLGASVKLVCTLSSGHSGHAIAWHQQQA
EKGPRFLMKVHSDGSHNKGDGIPDRFSGSVSGAERYLSIS
SLQSEDEADYYCQTWDTGLWVFGGGTKLTVL.

In particular embodiments, the Z538-03 antibody includes a lambda light chain.

In particular embodiments, a binding domain that binds T antigen of the Merkel cell polyomavirus includes the Z573-03 antibody. In particular embodiments, the Z573-03 antibody includes a variable heavy chain including the sequence:

(SEQ ID NO: 1313)
QVQLVHSGAEVKKPGASVKVSCKASGYTFTTYYIHWVRQA
PGQGLEWVGVLNPSRNRTIYAQKFQGRVTMTRDTSTSTVY
MELSSLRSEDTAVYYCARDAGLGPYYYYMDVWGKGTTVTV
SS

and a variable light chain sequence including the sequence:

(SEQ ID NO: 1314)
SYELTQPPSVSVSPGQTARITCSGDGLTKQYAFWYQQKPG
QAPVLVIYKDSERPSGIPERFSGSSSGTTVTLTISGVQAE
DEADYYCQSADRTDIYVVFGGGTKLTVL.

In particular embodiments, the Z573-03 antibody includes a lambda light chain.

In particular embodiments, a binding domain that binds T antigen of the Merkel cell polyomavirus includes the Z582-03 antibody. In particular embodiments, the Z582-03 antibody includes a variable heavy chain including the sequence:

(SEQ ID NO: 215)
QVQLVQSGAEVKKPGSSVKVSCKTSGGTFSSATLSWVRQA
PGQGLEWMGGITPMFTTANYAQKFQGRVTISADESTNTAY
MKLSSLRSEDTAVYYCARQFGHESRDSYYLYWGQGTLVTV
SS

and a variable light chain sequence including the sequence:

(SEQ ID NO: 216)
EIVLTQSPGTLSLSPGERATLSCRASQTLSSSYLAWYQQK
PGQAPRLLIYGASSRVAGVPVRFRGSGSGTDFTLTISRLE
PEDFAVYYCQQYGSSRTFGQGTKVEIK.

In particular embodiments, the Z582-03 antibody includes a kappa light chain.

In particular embodiments, a binding domain that binds T antigen of the Merkel cell polyomavirus includes the Z582-14 antibody. In particular embodiments, the Z582-14 antibody includes a variable heavy chain including the sequence:

(SEQ ID NO: 217)
QVQLQESGPGLVKPSETLALTCTVSGYSISSGYYWGWIRQ
PPGKGLEWIGSIYHSGSTYYNPSLNSRVTISVDTSKNQFS
LRLSSVTAADTAVYYCARSARAVVMDAFHIWSQGTVVTVS
S

and a variable light chain sequence including the sequence:

(SEQ ID NO: 218)
NFMLTQPHSVSESPGKTVTISCTGSSGSIASNYVQWYQQR
PGSAPTTVIYEDNQRPSGVPDRFSGSIDSSSNSASLTISG
LKTEDEADYYCQSYDNIHPWVFGGGTKVTVL

In particular embodiments, the Z582-14 antibody includes a lambda light chain.

In particular embodiments, a binding domain that binds T antigen of the Merkel cell polyomavirus includes the Z582-15 antibody. In particular embodiments, the Z582-15 antibody includes a variable heavy chain including the sequence:

(SEQ ID NO: 219)
QVQLQESGPGLVKPSETLALTCTVSGYSISSGYYWGWIRQ
PPGKGLEWIGSIYHSGSTYYNPSLNSRVTISVDTSKNQFS
LRLSSVTAADTAVYYCARSARAVVMDAFHIWSQGTVVTVS
S

and a variable light chain sequence including the sequence:

(SEQ ID NO: 220)
SYVLTQPPSVSVAPGKTARITCGGNNIGSKSVHWYQQKPG
QAPVLVIYYDSDRPSGIPERFSGSSSGNTATLTISRVEAG
DEADYYCQVWDGSTDHFYVFGTGTKVTVL

In particular embodiments, the Z582-15 antibody includes a lambda light chain.

In particular embodiments, a binding domain that binds T antigen of the Merkel cell polyomavirus includes the Z082-U1 antibody. In particular embodiments, the Z082-U1 antibody includes a variable heavy chain including the sequence:

(SEQ ID NO: 221)
QVQLQESGPGLVKPSQTLSLSCTVSGGSISSGEYYWSWIR
QPPGKGLEWIGYISYAGNTYYNPSLKSRLNISLDKSKNQF
SLKLTSVTAADTAIYYCARDFVGTNWYDRYAFHIWGQGTM
VTVSS

and a variable light chain sequence including the sequence:

(SEQ ID NO: 222)
LSLLTQGTGSWAQSALTQPRSVSGSPGHSVTISCTGTSSD
VGGYHYVSWYQQLPGKAPKLILYDVTKRPSGVPDRFSGSK
SGNTASLTISGLQAEDEADYFCCSYAGSYAFWVFGGGTKL
TVL.

In particular embodiments, the Z082-U1 antibody includes a lambda light chain.

In particular embodiments, a binding domain that binds T antigen of the Merkel cell polyomavirus includes the Z082-U5 antibody. In particular embodiments, the Z082-U5 antibody includes a variable heavy chain including the sequence:

(SEQ ID NO: 223)
CEVQLVESGGGLVRPGGSLRLSCGASGFNFIDYAMYWVRQ
APGKALEYVSSITNDAKSTFYGNSVKGRFTISRDNSKNTV
FLQMGSLRVEDMAVYYCARGRSHYYDSSGFDYWGQGALVT
VSP

and a variable light chain sequence including the sequence:

(SEQ ID NO: 224)
SYVLTQPPSVSVAPGQTARITCGGDSLGSKSVHWYQQKPG
QAPVLVVFDDSDRPSGIPERFSGSNSGNRGTLTISRVEAG
DEAVYYCQVWDAGALWVFGGGTELTVL.

In particular embodiments, the Z082-U5 antibody includes a lambda light chain.

In particular embodiments, a binding domain that binds T antigen of the Merkel cell polyomavirus includes the Z082-U6 antibody. In particular embodiments, the Z082-U6 antibody includes a variable heavy chain including the sequence:

(SEQ ID NO: 225)
QLQLQESGPGLVKPSETLSLTCSVSGDSISTTSYYWDWFR
QPPGKGLEWIGSINYSGNTYYSPLLKSRVTIASDTSKNQF
FLRLTSVTAADTAVYYCARRGVPAGMVPSWIDPWGQGTLV
AVSS

and a variable light chain sequence including the sequence:

(SEQ ID NO: 226)
DIQMTQSPSSLSASVGDRVTITCQASEDIRNYLNWYQQKP
GKAPKVLIYDASNLKTGVPSRFSGSGSRTHFTFTISSLQP
EDIATYYCQQYDTVPLTFGQGTKLEIK.

In particular embodiments, the Z082-U6 antibody includes a kappa light chain.

In particular embodiments, a binding domain that binds T antigen of the Merkel cell polyomavirus includes the W056-01 antibody. In particular embodiments, the W056-01 antibody includes a variable heavy chain including the sequence:

(SEQ ID NO: 227)
EVQLVQSGAEVKKPGESLKISCKGSGYHFPTYWIAWVRQM
PGKGLEWMGVIYPRDSDTKYSPSFQGQVTISVDKSITTAY
LQWNSLKASDTAMYYCARATGDYWGQGTLVTVSS

and a variable light chain sequence including the sequence:

(SEQ ID NO: 228)
DIQMTQSPSTLSASVGDRVTITCRAGENVSTWLTWYQQKP
GRAPERLMYKASTLETGVPSRFSGRGSGTEFILTISSLQP
DDFATYYCQQYKSYSRTFGQGTKVEI.

In particular embodiments, the W056-01 antibody includes a kappa light chain.

In particular embodiments, a binding domain that binds T antigen of the Merkel cell polyomavirus includes the W056-02 antibody. In particular embodiments, the W056-02 antibody includes a variable heavy chain including the sequence:

(SEQ ID NO: 229)
EVQLVQSGAEVKKPGESLKISCQGFGYSFPTYWIAWVRQM
PGKGLEWMGIIYPDDSDTTYSPSFQGQVTFSADKSTNTAY
LQWDSLKVSDSALYYCARGMGDHWGQGTLITVSS

and a variable light chain sequence including the sequence:

(SEQ ID NO: 230)
DVVLTQTPLSLSVTPGQPASISCKSSQSLLFGDGATYLYW
FLQRPGQSPHLLISKVSSRFSGVPDRFNGSGSGTDFTLKI
SRVEAEDFGVYYCMQALHMPRTFGQGTKVEIK.

In particular embodiments, the W056-02 antibody includes a kappa light chain.

In particular embodiments, a binding domain that binds T antigen of the Merkel cell polyomavirus includes the W917-01 antibody. In particular embodiments, the W917-01 antibody includes a variable heavy chain including the sequence:

(SEQ ID NO: 231)
EVOLVESGGGLVQPGGSLRLSCAASGFTVSSNYMSWVRQA
PGKGLEWVSVIYSGGSTYYADSVKGRFTISRDNSKNTLYL
QMNSLRAEDTAVYYCARDRKIVVRRGGLDIWGQGTMVTVS
S

and a variable light chain sequence including the sequence:
In particular embodiments, the W917-01 antibody includes a kappa light chain.

(SEQ ID NO: 232)
AIRMTQSPSSFSASTGDRVTITCRASQGISSYLAWYQQKPGKAPKLLIY
AASTLQSGVPSRFSGSGSGTDFTLTISCLQSEDFATYYCQQYYSYPHTF
GQGTRLEIK.

In particular embodiments, a binding domain that binds T antigen of the Merkel cell polyomavirus includes the W917-02 antibody. In particular embodiments, the W917-02 antibody includes a variable heavy chain including the sequence:

(SEQ ID NO: 233)
EVQLVESGGGLVQPGGSLRLSCAASGFTFSSYSMNWVRQA
PGKGLEWVSYISSSSSTIYYADSVKGRFTISRDNAKNSLY
LQMNSLRDEDTAVYYCARDGAAAGVNPWGQGTLVTVSS

and a variable light chain sequence including the sequence:

(SEQ ID NO: 234)
DIQMTQSPSTLSASVGDRVTITCRASQSISSWLAWYQQKPGKAPKLLIY
KASSLESGVPSRFSGSGSGTEFTLTISSLQPDDFATYYCQQYNSFPWTF
GQGTKVEIK.

In particular embodiments, the W917-02 antibody includes a kappa light chain.

In particular embodiments, a binding domain that binds T antigen of the Merkel cell polyomavirus includes the W917-03 antibody. In particular embodiments, the W917-03 antibody includes a variable heavy chain including the sequence:

(SEQ ID NO: 235)
EVQLVESGGGLVQPGGSLKLSCAASGFTFSGSAMHWVRQA
SGKGLEWVGRIRSKANSYATAYAASVKGRFTISRDDSKNT
AYLQMNSLKTEDTAVYYCTRLTDIFRLLGSNFYGMDVWGQ
GTTVTVSS

and a variable light chain sequence including the sequence:
In particular embodiments, the W917-03 antibody includes a lambda light chain.

(SEQ ID NO: 236)
SYELTQPPSVSVSPGQTARITCSGDALPKQYAYWYQQKPGQAPVLVIYK
DSERPSGIPERFSGSSSGTTVTLTISGVQAEDEADYYCQSADSSGTYVV
FGGGTKLTVL.

In particular embodiments, a binding domain that binds T antigen of the Merkel cell polyomavirus includes the Z504-01 antibody. In particular embodiments, the Z504-01 antibody includes a variable heavy chain including the sequence:

(SEQ ID NO: 237)
EVQLLESGGGLVQPGGSLRLSCAASRFTFSRYAMTWVRQA
PGKGLEWVSAISSTGDNPYYADSVKGRFTISRDNSKNILY
LQMNSLRAEDTAVYYCAKDQGGADNNGYYYNFDYWGQGTL
VTVSS

and a variable light chain sequence including the sequence:

(SEQ ID NO: 238)
SYVLTQPSSVSVAPGKTARITCGGNDIGSKSVHWYQQKPGQAPVQVIYY
DSDRPSGIPERFSGSNSGNTATLTISRVEAGDEADYYCQVWDSSSDHVV
FGGGTKLTVL.

In particular embodiments, the Z504-01 antibody includes a lambda light chain.

In particular embodiments, a binding domain that binds T antigen of the Merkel cell polyomavirus includes the Z504-02 antibody. In particular embodiments, the Z504-02 antibody includes a variable heavy chain including the sequence:

(SEQ ID NO: 239)
QVQLLESGGGLVQPGGSLRLSCAASGFSFSTYAMNWVRQAPGKGLEWVS
LFSGSGGDTYYADSVKGRFTISRDNSKNTLYLQMKNLRVEDTAIYYCAK
DLGVMFFGAFDIWGQGTMVTVSS

and a variable light chain sequence including the sequence:
In particular embodiments, the Z504-02 antibody includes a kappa light chain.

(SEQ ID NO: 240)
DIQMTQSPSILSASVGDRVTITCRASQSISSWLAWYQQKPGKAPKLLIY
KASSLQSGVPSRFSGSGSGTEFTLTISSLQPDDFATYYCQQYNDYSVTF
GQGTKLEIK.

In particular embodiments, a binding domain that binds T antigen of the Merkel cell polyomavirus includes the Z538-01 antibody. In particular embodiments, the Z538-01 antibody includes a variable heavy chain including the sequence:

(SEQ ID NO: 241)
EVQLVESGGGLVQPGGSLRLSCAASGFTFTNYWMHWVRQAPGKGLVWVS
RLNSDGSITGYADSVKGRFTISRDNPKNTLYLQMNSLRAEDTAIYYCAR
VGLRTLPAIENFDYWGQGTLVTVSS

and a variable light chain sequence including the sequence:

(SEQ ID NO: 242)
DIQMTQSPSSLSASVGDRVTITCRASQSISIYLNWYQQKPGKPPKLLIY
AASSLQSGVPSRFSGSGSGTDFTLTISSLHPEDFATYYCQQSYRAVLTF
GGGTKVEIK.

In particular embodiments, the Z538-01 antibody includes a kappa light chain.

In particular embodiments, a binding domain that binds T antigen of the Merkel cell polyomavirus includes the Z538-02 antibody. In particular embodiments, the Z538-02 antibody includes a variable heavy chain including the sequence:

(SEQ ID NO: 243)
EVQLVESGGGLVQPGGSRRLSCAASGFIFSYYWMSWVRQAPGKGLEWVA
NIKEDGSEKYYVDSVKGRFTISRDNAKTSLFLQMNTLRAEDTAVYYCAR
APLRNFDWPLDVWGQGALVTVSS

and a variable light chain sequence including the sequence:

(SEQ ID NO: 244)
QSALTQPRSVSGSPGQSLSISCTGTSSDVGANDYVSWYQQHPGRAPKLI
IYDVSKRTSGVPHRFSGSKSGNTASLTISGLQAEDEADYYCCSYGGRYV
FGTGTEVTVL.

In particular embodiments, the Z538-02 antibody includes a lambda light chain.

In particular embodiments, a binding domain that binds T antigen of the Merkel cell polyomavirus includes the Z573-01 antibody. In particular embodiments, the Z573-01 antibody includes a variable heavy chain including the sequence:

(SEQ ID NO: 245)
EVQLVQSGAEVKKPGESLKISCKGSGYKFSSYWIAWVCQMPGKGLEWMG
IIYPDDSDTTYSPSFQGQVTISADKSINTAYLQWNSLKASDTAMYYCVR
GGSAPHWGQGTLVTVSS

and a variable light chain sequence including the sequence:

(SEQ ID NO: 246)
DIVMTQSPDSLAVSLGERATINCKSSQSVLYSSNNKNYLTWYQQKPGQP
PKLLIYWASTRESGVPDRFSGSGSGTDFTLTINSLQAEDVAVYYCQQHY
TTPWTFGQGTKVEIQ.

In particular embodiments, the Z573-01 antibody includes a kappa light chain.

In particular embodiments, a binding domain that binds T antigen of the Merkel cell polyomavirus includes the Z573-02 antibody. In particular embodiments, the Z573-02 antibody includes a variable heavy chain including the sequence:

(SEQ ID NO: 247)
QVQLVQSGAEVKKPGASVKVSCKASGYTFNTFYMHWVRQAPGQGLEWVG
VINPSRGRTIYAQKFQGRVTMTRDTSTSTVYMELSSLRSEDTAVYYCAR
DAGLGPYYYYMDVWGKGTTVTVSS

and a variable light chain sequence including the sequence:

(SEQ ID NO: 248)
SYELTQPPSVSVSPGQTARITCSGDGLTKQYAFWYQQKPGQAPVLVIYK
DSERPSGIPERFSGSSSGTTVTLTISGVQAEDEADYYCQSADRSDIYVV
FGGGTKLTVL.

In particular embodiments, the Z573-02 antibody includes a lambda light chain.

Referring to the antibodies provided herein, the following CDR sets are provided. A CDR set refers to 3 light chain CDRs and 3 heavy chain CDRs that together result in binding to the T antigen of the Merkel cell polyomavirus.

TABLE 1
Antibody CDR Sequences.

| | CDR | | | SEQ ID |
|---|---|---|---|---|
| Antibody | Definition | CDR | Sequence | NO: |
| 1C10 | North | CDRH1 | KASGYTFTIYYMH | 57 |
| | | CDRH2 | IINPSGGRAT | 58 |
| | | CDRH3 | ARDRGGQNCTATRCPHYFFSGMDV | 59 |
| | | CDRL1 | SGDELSKQYAY | 60 |
| | | CDRL2 | YKDTERPS | 61 |
| | | CDRL3 | QSADTTVF | 62 |
| | Kabat | CDRH1 | IYYMH | 63 |
| | | CDRH2 | IINPSGGRATYAEKLQG | 64 |
| | | CDRH3 | DRGGQNCTATRCPHYFFSGMDV | 65 |
| | | CDRL1 | SGDELSKQYAY | 60 |
| | | CDRL2 | KDTERPS | 66 |
| | | CDRL3 | QSADTTVF | 62 |
| | IMGT | CDRH1 | GYTFTIYY | 67 |
| | | CDRH2 | INPSGGRA | 68 |
| | | CDRH3 | ARDRGGQNCTATRCPHYFFSGMDV | 59 |
| | | CDRL1 | ELSKQY | 69 |
| | | CDRL2 | KDT | N/A |
| | | CDRL3 | QSADTTVF | 62 |
| | Chothia | CDRH1 | GYTFTIY | 70 |
| | | CDRH2 | WMGIINPSGGRAT | 71 |
| | | CDRH3 | DRGGQNCTATRCPHYFFSGMDV | 65 |
| | | CDRL1 | SGDELSKQYAY | 60 |
| | | CDRL2 | KDTERPS | 66 |
| | | CDRL3 | QSADTTVF | 62 |
| | Contact | CDRH1 | TIYYMH | 72 |
| | | CDRH2 | WMGIINPSGGRAT | 71 |
| | | CDRH3 | ARDRGGQNCTATRCPHYFFSGMD | 73 |
| | | CDRL1 | KQYAYWY | 74 |
| | | CDRL2 | LMIYKDTERP | 75 |
| | | CDRL3 | QSADTTV | 76 |
| 3B09 | North | CDRH1 | AVSVGSISSSSYYWG | 79 |
| | | CDRH2 | SISHSGNTY | 80 |
| | | CDRH3 | ARQPQYFYDSSGHSLAYYGLDV | 81 |
| | | CDRL1 | TGASSDFGGYNSVS | 82 |
| | | CDRL2 | YDVHNRPS | 83 |
| | | CDRL3 | SSYTTSTFWV | 84 |
| | Kabat | CDRH1 | SSSYYWG | 85 |
| | | CDRH2 | SISHSGNTYYNPSLKS | 86 |
| | | CDRH3 | QPQYFYDSSGHSLAYYGLDV | 87 |
| | | CDRL1 | TGASSDFGGYNSVS | 82 |
| | | CDRL2 | DVHNRPS | 88 |
| | | CDRL3 | SSYTTSTFWV | 84 |
| | IMGT | CDRH1 | VGSISSSSYY | 89 |
| | | CDRH2 | ISHSGNT | 90 |
| | | CDRH3 | ARQPQYFYDSSGHSLAYYGLDV | 81 |
| | | CDRL1 | SSDFGGYNS | 91 |
| | | CDRL2 | DVH | N/A |
| | | CDRL3 | SSYTTSTFWV | 84 |
| | Chothia | CDRH1 | VGSISSSSY | 92 |
| | | CDRH2 | SHSGN | 93 |
| | | CDRH3 | QPQYFYDSSGHSLAYYGLDV | 87 |
| | | CDRL1 | TGASSDFGGYNSVS | 82 |
| | | CDRL2 | DVHNRPS | 88 |
| | | CDRL3 | SSYTTSTFWV | 84 |
| | Contact | CDRH1 | SSSSYYWG | 94 |
| | | CDRH2 | WIGSISHSGNTY | 95 |
| | | CDRH3 | ARQPQYFYDSSGHSLAYYGLD | 96 |
| | | CDRL1 | YNSVSWY | 97 |
| | | CDRL2 | LMIYDVHNRP | 98 |
| | | CDRL3 | SSYTTSTFW | 99 |
| 1B09 | North | CDRH1 | AASGFNFSNYAMH | 102 |
| | | CDRH2 | VLSYDGSNEY | 103 |
| | | CDRH3 | ARPRGSARRRFFYFDH | 104 |
| | | CDRL1 | TGTINDVGGFDLVS | 105 |
| | | CDRL2 | YDVSDRPS | 106 |
| | | CDRL3 | SSYTTSTSLLYV | 107 |
| | Kabat | CDRH1 | NYAMH | 108 |
| | | CDRH2 | VLSYDGSNEYYAGSVRG | 109 |
| | | CDRH3 | PRGSARRRFFYFDH | 110 |
| | | CDRL1 | TGTINDVGGFDLVS | 105 |
| | | CDRL2 | DVSDRPS | 111 |
| | | CDRL3 | SSYTTSTSLLYV | 107 |
| | IMGT | CDRH1 | GFNFSNYA | 112 |
| | | CDRH2 | LSYDGSNE | 113 |
| | | CDRH3 | ARPRGSARRRFFYFDH | 104 |
| | | CDRL1 | INDVGGFDL | 114 |
| | | CDRL2 | DVS | N/A |
| | | CDRL3 | SSYTTSTSLLYV | 107 |
| | Chothia | CDRH1 | GFNFSNY | 115 |
| | | CDRH2 | SYDGSN | 116 |
| | | CDRH3 | PRGSARRRFFYFDH | 110 |
| | | CDRL1 | TGTINDVGGFDLVS | 105 |
| | | CDRL2 | DVSDRPS | 111 |
| | | CDRL3 | SSYTTSTSLLYV | 107 |
| | Contact | CDRH1 | SNYAMH | 117 |
| | | CDRH2 | WVAVLSYDGSNEY | 118 |
| | | CDRH3 | ARPRGSARRRFFYFD | 119 |
| | | CDRL1 | VGGFDLVSWY | 120 |
| | | CDRL2 | VLIYDVSDRP | 121 |
| | | CDRL3 | SSYTTSTSLLY | 122 |
| 1G04 | North | CDRH1 | AVSGDSISGGVYSWS | 125 |
| | | CDRH2 | YIDQSGSTY | 126 |
| | | CDRH3 | ARASPMMVVLN | 127 |
| | | CDRL1 | RASQSVDTWLA | 128 |
| | | CDRL2 | HLASNLDS | 129 |
| | | CDRL3 | QQYHTHSGT | 130 |
| | Kabat | CDRH1 | GGVYSWS | 131 |
| | | CDRH2 | YIDQSGSTYYSPSLKS | 132 |
| | | CDRH3 | ASPMMVVLN | 133 |
| | | CDRL1 | RASQSVDTWLA | 128 |
| | | CDRL2 | LASNLDS | 134 |
| | | CDRL3 | QQYHTHSGT | 130 |
| | IMGT | CDRH1 | GDSISGGVYS | 135 |
| | | CDRH2 | IDQSGST | 136 |
| | | CDRH3 | ARASPMMVVLN | 127 |
| | | CDRL1 | QSVDTW | 137 |
| | | CDRL2 | LAS | N/A |
| | | CDRL3 | QQYHTHSGT | 130 |
| | Chothia | CDRH1 | GDSISGGVY | 138 |
| | | CDRH2 | DQSGS | 139 |
| | | CDRH3 | ASPMMVVLN | 133 |
| | | CDRL1 | RASQSVDTWLA | 128 |
| | | CDRL2 | LASNLDS | 134 |
| | | CDRL3 | QQYHTHSGT | 130 |
| | Contact | CDRH1 | SGGVYSWS | 140 |
| | | CDRH2 | WIGYIDQSGSTY | 141 |
| | | CDRH3 | ARASPMMVVL | 142 |
| | | CDRL1 | DTWLAWY | 143 |
| | | CDRL2 | LLIHLASNLD | 144 |
| | | CDRL3 | QQYHTHSG | 145 |
| 2H04 | North | CDRH1 | AGTGFTFNVYAMN | 148 |
| | | CDRH2 | SISPGGTSTY | 149 |
| | | CDRH3 | ARDSIFGVIMGGLDP | 150 |
| | | CDRL1 | GVRNHITDGVH | 151 |
| | | CDRL2 | YDDRDRPS | 152 |
| | | CDRL3 | QVWDRNRDQVI | 153 |
| | Kabat | CDRH1 | VYAMN | 154 |
| | | CDRH2 | SISPGGTSTYYADSVKG | 155 |
| | | CDRH3 | DSIFGVIMGGLDP | 156 |
| | | CDRL1 | GVRNHITDGVH | 151 |
| | | CDRL2 | DDRDRPS | 157 |
| | | CDRL3 | QVWDRNRDQVI | 153 |
| | IMGT | CDRH1 | GFTFNVYA | 158 |
| | | CDRH2 | ISPGGTST | 159 |
| | | CDRH3 | ARDSIFGVIMGGLDP | 150 |
| | | CDRL1 | NHITDG | 160 |
| | | CDRL2 | DDR | N/A |
| | | CDRL3 | QVWDRNRDQVI | 153 |
| | Chothia | CDRH1 | GFTFNVY | 161 |
| | | CDRH2 | SPGGTS | 162 |
| | | CDRH3 | DSIFGVIMGGLDP | 156 |
| | | CDRL1 | GVRNHITDGVH | 151 |
| | | CDRL2 | DDRDRPS | 157 |
| | | CDRL3 | QVWDRNRDQVI | 153 |
| | Contact | CDRH1 | NVYAMN | 163 |
| | | CDRH2 | WVSSISPGGTSTY | 164 |
| | | CDRH3 | ARDSIFGVIMGGLD | 165 |
| | | CDRL1 | TDGVHWY | 166 |
| | | CDRL2 | LVVYDDRDRP | 167 |
| | | CDRL3 | QVWDRNRDQV | 168 |
| W056-03 | North | CDRH1 | AASGVTFSRFDMN | 250 |
| | | CDRH2 | YISSSGTTIY | 251 |
| | | CDRH3 | ARDDYTGWYYFDS | 252 |
| | | CDRL1 | TGSSSNIGAGFDVH | 253 |
| | | CDRL2 | YGNSNRPS | 254 |
| | | CDRL3 | QSYDSSLYV | 255 |
| | Kabat | CDRH1 | RFDMN | 256 |
| | | CDRH2 | YISSSGTTIYYADSVKG | 257 |
| | | CDRH3 | DDYTGWYYFDS | 258 |
| | | CDRL1 | TGSSSNIGAGFDVH | 253 |
| | | CDRL2 | GNSNRPS | 259 |
| | | CDRL3 | QSYDSSLYV | 255 |
| | IMGT | CDRH1 | GVTFSRFD | 260 |
| | | CDRH2 | ISSSGTTI | 261 |
| | | CDRH3 | ARDDYTGWYYFDS | 252 |
| | | CDRL1 | SSNIGAGFD | 262 |
| | | CDRL2 | GN | — |
| | | CDRL3 | QSYDSSLYV | 255 |
| | Chothia | CDRH1 | GVTFSRF | 263 |
| | | CDRH2 | SSSGTT | 264 |
| | | CDRH3 | DDYTGWYYFDS | 258 |
| | | CDRL1 | TGSSSNIGAGFDVH | 253 |
| | | CDRL2 | GNSNRPS | 259 |
| | | CDRL3 | QSYDSSLYV | 255 |
| | Contact | CDRH1 | SRFDMN | 265 |
| | | CDRH2 | WVSYISSSGTTIY | 266 |
| | | CDRH3 | ARDDYTGWYYFD | 267 |
| | | CDRL1 | IGAGFDVHWY | 268 |
| | | CDRL2 | LLIYGNSNRP | 269 |
| | | CDRL3 | QSYDSSLY | 270 |
| W056-04 | North | CDRH1 | TFSGFSLSTSGMCVS | 271 |
| | | CDRH2 | RIDWDDDKY | 272 |
| | | CDRH3 | ARQELVGAWFDP | 273 |
| | | CDRL1 | TGTSGDVGGYNYVS | 274 |
| | | CDRL2 | YEVSNRPS | 275 |
| | | CDRL3 | ISYTSSSISCV | 276 |
| | Kabat | CDRH1 | TSGMCVS | 277 |
| | | CDRH2 | RIDWDDDKYYNTSLKT | 278 |
| | | CDRH3 | QELVGAWFDP | 279 |
| | | CDRL1 | TGTSGDVGGYNYVS | 274 |
| | | CDRL2 | EVSNRPS | 280 |
| | | CDRL3 | ISYTSSSISCV | 276 |
| | IMGT | CDRH1 | GFSLSTSGMC | 281 |
| | | CDRH2 | IDWDDDK | 282 |
| | | CDRH3 | ARQELVGAWFDP | 273 |
| | | CDRL1 | SGDVGGYNY | 283 |
| | | CDRL2 | EV | — |
| | | CDRL3 | ISYTSSSISCV | 276 |
| | Chothia | CDRH1 | GFSLSTSGM | 284 |
| | | CDRH2 | DWDDD | 285 |
| | | CDRH3 | QELVGAWFDP | 279 |
| | | CDRL1 | TGTSGDVGGYNYVS | 274 |
| | | CDRL2 | EVSNRPS | 280 |
| | | CDRL3 | ISYTSSSISCV | 276 |
| | Contact | CDRH1 | STSGMCVS | 286 |
| | | CDRH2 | WLARIDWDDDKY | 287 |
| | | CDRH3 | ARQELVGAWFD | 288 |
| | | CDRL1 | VGGYNYVSWY | 289 |
| | | CDRL2 | LIIYEVSNRP | 290 |
| | | CDRL3 | ISYTSSSISC | 291 |
| W056-05 | North | CDRH1 | AASGFTFSSYGMH | 292 |
| | | CDRH2 | VISYDGSTKY | 293 |
| | | CDRH3 | AKDGTAVLVDRLDY | 294 |
| | | CDRL1 | RASQGISSYLA | 295 |
| | | CDRL2 | YAASTLQS | 296 |
| | | CDRL3 | QQLNSYPLT | 297 |
| | Kabat | CDRH1 | SYGMH | 298 |
| | | CDRH2 | VISYDGSTKYYADSVKG | 299 |
| | | CDRH3 | DGTAVLVDRLDY | 300 |
| | | CDRL1 | RASQGISSYLA | 295 |
| | | CDRL2 | AASTLQS | 301 |
| | | CDRL3 | QQLNSYPLT | 297 |
| | IMGT | CDRH1 | GFTFSSYG | 302 |
| | | CDRH2 | ISYDGSTK | 303 |
| | | CDRH3 | AKDGTAVLVDRLDY | 294 |
| | | CDRL1 | QGISSY | 304 |
| | | CDRL2 | AA | — |
| | | CDRL3 | QQLNSYPLT | 297 |
| | Chothia | CDRH1 | GFTFSSY | 305 |
| | | CDRH2 | SYDGST | 306 |
| | | CDRH3 | DGTAVLVDRLDY | 300 |
| | | CDRL1 | RASQGISSYLA | 295 |
| | | CDRL2 | AASTLQS | 301 |
| | | CDRL3 | QQLNSYPLT | 297 |
| | Contact | CDRH1 | SSYGMH | 307 |
| | | CDRH2 | WVAVISYDGSTKY | 308 |
| | | CDRH3 | AKDGTAVLVDRLD | 309 |
| | | CDRL1 | SSYLAWY | 310 |
| | | CDRL2 | LLIYAASTLQ | 311 |
| | | CDRL3 | QQLNSYPL | 312 |
| W056-06 | North | CDRH1 | AASGYDFDTYWIA | 313 |
| | | CDRH2 | VIYPDDSDTK | 314 |
| | | CDRH3 | ARATGDY | 315 |
| | | CDRL1 | RASQNVSTWLT | 316 |
| | | CDRL2 | YEASTLQS | 317 |
| | | CDRL3 | QQYRSYSRT | 318 |
| | Kabat | CDRH1 | TYWIA | 319 |
| | | CDRH2 | VIYPDDSDTKYSPSFQG | 320 |
| | | CDRH3 | ATGDY | 321 |
| | | CDRL1 | RASQNVSTWLT | 316 |
| | | CDRL2 | EASTLQS | 322 |
| | | CDRL3 | QQYRSYSRT | 318 |
| | IMGT | CDRH1 | GYDFDTYW | 323 |
| | | CDRH2 | IYPDDSDT | 324 |
| | | CDRH3 | ARATGDY | 315 |
| | | CDRL1 | QNVSTW | 325 |
| | | CDRL2 | EA | — |
| | | CDRL3 | QQYRSYSRT | 318 |
| | Chothia | CDRH1 | GYDFDTY | 326 |
| | | CDRH2 | YPDDSD | 327 |
| | | CDRH3 | ATGDY | 321 |
| | | CDRL1 | RASQNVSTWLT | 316 |
| | | CDRL2 | EASTLQS | 322 |
| | | CDRL3 | QQYRSYSRT | 318 |
| | Contact | CDRH1 | DTYWIA | 323 |
| | | CDRH2 | WMGVIYPDDSDTK | 324 |
| | | CDRH3 | ARATGD | 325 |
| | | CDRL1 | STWLTWY | 326 |
| | | CDRL2 | RLIYEASTLQ | 327 |
| | | CDRL3 | QQYRSYSR | 335 |
| W056-08 | North | CDRH1 | TFSGFSLSTSGMCVS | 271 |
| | | CDRH2 | RIDWDDDKY | 272 |
| | | CDRH3 | ARQELVGAWFDP | 273 |
| | | CDRL1 | TGTSGDVGGYNYVS | 274 |
| | | CDRL2 | YEVSNRPS | 275 |
| | | CDRL3 | ISYTSSSISCV | 276 |
| | Kabat | CDRH1 | TSGMCVS | 277 |
| | | CDRH2 | RIDWDDDKYYNTSLKT | 278 |
| | | CDRH3 | QELVGAWFDP | 279 |
| | | CDRL1 | TGTSGDVGGYNYVS | 274 |
| | | CDRL2 | EVSNRPS | 280 |
| | | CDRL3 | ISYTSSSISCV | 276 |
| | IMGT | CDRH1 | GFSLSTSGMC | 281 |
| | | CDRH2 | IDWDDDK | 282 |
| | | CDRH3 | ARQELVGAWFDP | 273 |
| | | CDRL1 | SGDVGGYNY | 283 |
| | | CDRL2 | EV | — |
| | | CDRL3 | ISYTSSSISCV | 276 |
| | Chothia | CDRH1 | GFSLSTSGM | 284 |
| | | CDRH2 | DWDDD | 285 |
| | | CDRH3 | QELVGAWFDP | 279 |
| | | CDRL1 | TGTSGDVGGYNYVS | 274 |
| | | CDRL2 | EVSNRPS | 280 |
| | | CDRL3 | ISYTSSSISCV | 276 |
| | Contact | CDRH1 | STSGMCVS | 286 |
| | | CDRH2 | WLARIDWDDDKY | 287 |
| | | CDRH3 | ARQELVGAWFD | 288 |
| | | CDRL1 | VGGYNYVSWY | 289 |
| | | CDRL2 | LIIYEVSNRP | 290 |
| | | CDRL3 | ISYTSSSISC | 291 |
| W056-09 | North | CDRH1 | AGSGYDFNTYWIA | 328 |
| | | CDRH2 | VIYPDDSDTK | 314 |
| | | CDRH3 | ARATGDY | 315 |
| | | CDRL1 | RASQNVSRWLT | 329 |
| | | CDRL2 | YEASTLQS | 317 |
| | | CDRL3 | QQYRSYSRT | 318 |
| | Kabat | CDRH1 | TYWIA | 319 |
| | | CDRH2 | VIYPDDSDTKYSPSFQG | 320 |
| | | CDRH3 | ATGDY | 321 |
| | | CDRL1 | RASQNVSRWLT | 329 |
| | | CDRL2 | EASTLQS | 322 |
| | | CDRL3 | QQYRSYSRT | 318 |
| | IMGT | CDRH1 | GYDFNTYW | 330 |
| | | CDRH2 | IYPDDSDT | 324 |
| | | CDRH3 | ARATGDY | 315 |
| | | CDRL1 | QNVSRW | 331 |
| | | CDRL2 | EA | — |
| | | CDRL3 | QQYRSYSRT | 318 |
| | Chothia | CDRH1 | GYDFNTY | 332 |
| | | CDRH2 | YPDDSD | 327 |
| | | CDRH3 | ATGDY | 321 |
| | | CDRL1 | RASQNVSRWLT | 329 |
| | | CDRL2 | EASTLQS | 322 |
| | | CDRL3 | QQYRSYSRT | 318 |
| | Contact | CDRH1 | NTYWIA | 333 |
| | | CDRH2 | WMGVIYPDDSDTK | 324 |
| | | CDRH3 | ARATGD | 325 |
| | | CDRL1 | SRWLTWY | 334 |
| | | CDRL2 | RLIYEASTLQ | 327 |
| | | CDRL3 | QQYRSYSR | 335 |
| W056-10 | North | CDRH1 | KASGATFSSYAIS | 336 |
| | | CDRH2 | RVTPIIDTTN | 337 |
| | | CDRH3 | ATRGTYYDFWPSMDV | 338 |
| | | CDRL1 | RASQDIRNYLA | 339 |
| | | CDRL2 | YAASSLHS | 340 |
| | | CDRL3 | QQYNSYPLT | 341 |
| | Kabat | CDRH1 | SYAIS | 342 |
| | | CDRH2 | RVTPIIDTTNYAQKFQG | 343 |
| | | CDRH3 | RGTYYDFWPSMDV | 344 |
| | | CDRL1 | RASQDIRNYLA | 339 |
| | | CDRL2 | AASSLHS | 345 |
| | | CDRL3 | QQYNSYPLT | 341 |
| | IMGT | CDRH1 | GATFSSYA | 346 |
| | | CDRH2 | VTPIIDTT | 347 |
| | | CDRH3 | ATRGTYYDFWPSMDV | 338 |
| | | CDRL1 | QDIRNY | 348 |
| | | CDRL2 | AA | — |
| | | CDRL3 | QQYNSYPLT | 341 |
| | Chothia | CDRH1 | GATFSSY | 349 |
| | | CDRH2 | TPIIDT | 350 |
| | | CDRH3 | RGTYYDFWPSMDV | 344 |
| | | CDRL1 | RASQDIRNYLA | 339 |
| | | CDRL2 | AASSLHS | 345 |
| | | CDRL3 | QQYNSYPLT | 341 |
| | Contact | CDRH1 | SSYAIS | 351 |
| | | CDRH2 | WLGRVTPIIDTTN | 352 |
| | | CDRH3 | ATRGTYYDFWPSMD | 353 |
| | | CDRL1 | RNYLAWF | 354 |
| | | CDRL2 | SLIYAASSLH | 355 |
| | | CDRL3 | QQYNSYPL | 356 |
| Z156-02 | North | CDRH1 | AVYGGSFSGYYWS | 357 |
| | | CDRH2 | EINHSGSTN | 358 |
| | | CDRH3 | ASMSGSYYGAFDY | 359 |
| | | CDRL1 | SGDALPKQYAY | 360 |
| | | CDRL2 | YKDSERPS | 361 |
| | | CDRL3 | QSADSSGTYYV | 362 |
| | Kabat | CDRH1 | GYYWS | 363 |
| | | CDRH2 | EINHSGSTNYNPSLKS | 364 |
| | | CDRH3 | MSGSYYGAFDY | 365 |
| | | CDRL1 | SGDALPKQYAY | 360 |
| | | CDRL2 | KDSERPS | 366 |
| | | CDRL3 | QSADSSGTYYV | 362 |
| | IMGT | CDRH1 | GGSFSGYY | 367 |
| | | CDRH2 | INHSGST | 368 |
| | | CDRH3 | ASMSGSYYGAFDY | 359 |
| | | CDRL1 | ALPKQY | 369 |
| | | CDRL2 | KD | — |
| | | CDRL3 | QSADSSGTYYV | 362 |
| | Chothia | CDRH1 | GGSFSGY | 370 |
| | | CDRH2 | NHSGS | 371 |
| | | CDRH3 | MSGSYYGAFDY | 365 |
| | | CDRL1 | SGDALPKQYAY | 360 |
| | | CDRL2 | KDSERPS | 366 |
| | | CDRL3 | QSADSSGTYYV | 362 |
| | Contact | CDRH1 | SGYYWS | 372 |
| | | CDRH2 | WIGEINHSGSTN | 373 |
| | | CDRH3 | ASMSGSYYGAFD | 374 |
| | | CDRL1 | KQYAYWY | 74 |
| | | CDRL2 | LVIYKDSERP | 375 |
| | | CDRL3 | QSADSSGTYY | 376 |
| Z156-03 | North | CDRH1 | TVSGGSISSSSYYWG | 377 |
| | | CDRH2 | SIYYSGSTY | 378 |
| | | CDRH3 | ASIIAALFDY | 379 |
| | | CDRL1 | SGDVLAKKYAR | 380 |
| | | CDRL2 | YKDSERPS | 361 |
| | | CDRL3 | YSAADNNWV | 381 |
| | Kabat | CDRH1 | SSSYYWG | 85 |
| | | CDRH2 | SIYYSGSTYYNPSLKS | 382 |
| | | CDRH3 | IIAALFDY | 383 |
| | | CDRL1 | SGDVLAKKYAR | 380 |
| | | CDRL2 | KDSERPS | 366 |
| | | CDRL3 | YSAADNNWV | 381 |
| | IMGT | CDRH1 | GGSISSSSYY | 384 |
| | | CDRH2 | IYYSGST | 385 |
| | | CDRH3 | ASIIAALFDY | 379 |
| | | CDRL1 | VLAKKY | 386 |
| | | CDRL2 | KD | — |
| | | CDRL3 | YSAADNNWV | 381 |
| | Chothia | CDRH1 | GGSISSSSY | 387 |
| | | CDRH2 | YYSGS | 388 |
| | | CDRH3 | IIAALFDY | 383 |
| | | CDRL1 | SGDVLAKKYAR | 380 |
| | | CDRL2 | KDSERPS | 366 |
| | | CDRL3 | YSAADNNWV | 381 |
| | Contact | CDRH1 | SSSSYYWG | 94 |
| | | CDRH2 | WIGSIYYSGSTY | 389 |
| | | CDRH3 | ASIIAALFD | 390 |
| | | CDRL1 | KKYARWF | 391 |
| | | CDRL2 | LVIYKDSERP | 375 |
| | | CDRL3 | YSAADNNW | 392 |
| Z249-01 | North | CDRH1 | AASGFTFKHYSMH | 393 |
| | | CDRH2 | HISYDGSKTN | 394 |
| | | CDRH3 | VRDRTTGHDFGFDY | 395 |
| | | CDRL1 | SGDALSKKYAY | 396 |
| | | CDRL2 | FKDNERPA | 397 |
| | | CDRL3 | QSTDSDTSYVI | 398 |
| | Kabat | CDRH1 | HYSMH | 399 |
| | | CDRH2 | HISYDGSKTNYADSVKG | 400 |
| | | CDRH3 | DRTTGHDFGFDY | 401 |
| | | CDRL1 | SGDALSKKYAY | 396 |
| | | CDRL2 | KDNERPA | 402 |
| | | CDRL3 | QSTDSDTSYVI | 398 |
| | IMGT | CDRH1 | GFTFKHYS | 403 |
| | | CDRH2 | ISYDGSKT | 404 |
| | | CDRH3 | VRDRTTGHDFGFDY | 395 |
| | | CDRL1 | ALSKKY | 405 |
| | | CDRL2 | KD | — |
| | | CDRL3 | QSTDSDTSYVI | 398 |
| | Chothia | CDRH1 | GFTFKHY | 406 |
| | | CDRH2 | SYDGSK | 407 |
| | | CDRH3 | DRTTGHDFGFDY | 401 |
| | | CDRL1 | SGDALSKKYAY | 396 |
| | | CDRL2 | KDNERPA | 402 |
| | | CDRL3 | QSTDSDTSYVI | 398 |
| | Contact | CDRH1 | KHYSMH | 408 |
| | | CDRH2 | WVAHISYDGSKTN | 409 |
| | | CDRH3 | VRDRTTGHDFGFD | 410 |
| | | CDRL1 | KKYAYWY | 411 |
| | | CDRL2 | LVMFKDNERP | 412 |
| | | CDRL3 | QSTDSDTSYV | 413 |
| Z249-02 | North | CDRH1 | KTSGGFFSSYRIN | 414 |
| | | CDRH2 | GLVPIFETAK | 415 |
| | | CDRH3 | ARGSGSGLTYFQT | 416 |
| | | CDRL1 | RASQSITTYYLA | 417 |
| | | CDRL2 | YGASSRAT | 418 |
| | | CDRL3 | QQYDSSPT | 419 |
| | Kabat | CDRH1 | SYRIN | 420 |
| | | CDRH2 | GLVPIFETAKYAQKFQG | 421 |
| | | CDRH3 | GSGSGLTYFQT | 422 |
| | | CDRL1 | RASQSITTYYLA | 417 |
| | | CDRL2 | GASSRAT | 423 |
| | | CDRL3 | QQYDSSPT | 419 |
| | IMGT | CDRH1 | GGFFSSYR | 424 |
| | | CDRH2 | LVPIFETA | 425 |
| | | CDRH3 | ARGSGSGLTYFQT | 416 |
| | | CDRL1 | QSITTYY | 426 |
| | | CDRL2 | GA | — |
| | | CDRL3 | QQYDSSPT | 419 |
| | Chothia | CDRH1 | GGFFSSY | 427 |
| | | CDRH2 | VPIFET | 428 |
| | | CDRH3 | GSGSGLTYFQT | 422 |
| | | CDRL1 | RASQSITTYYLA | 417 |
| | | CDRL2 | GASSRAT | 423 |
| | | CDRL3 | QQYDSSPT | 419 |
| | Contact | CDRH1 | SSYRIN | 429 |
| | | CDRH2 | WMGGLVPIFETAK | 430 |
| | | CDRH3 | ARGSGSGLTYFQ | 431 |
| | | CDRL1 | TTYYLAWY | 432 |
| | | CDRL2 | LLIYGASSRA | 433 |
| | | CDRL3 | QQYDSSP | 434 |
| Z249-03 | North | CDRH1 | TVSGGSITSISHYWG | 435 |
| | | CDRH2 | SMYYSGTTY | 436 |
| | | CDRH3 | ARHDVSRFDP | 437 |
| | | CDRL1 | TGTSSDVGGYNYVS | 438 |
| | | CDRL2 | YDVTNRPS | 439 |
| | | CDRL3 | SSYTTSTWV | 440 |
| | Kabat | CDRH1 | SISHYWG | 441 |
| | | CDRH2 | SMYYSGTTYYNPSLKS | 442 |
| | | CDRH3 | HDVSRFDP | 443 |
| | | CDRL1 | TGTSSDVGGYNYVS | 438 |
| | | CDRL2 | DVTNRPS | 444 |
| | | CDRL3 | SSYTTSTWV | 440 |
| | IMGT | CDRH1 | GGSITSISHY | 445 |
| | | CDRH2 | MYYSGTT | 446 |
| | | CDRH3 | ARHDVSRFDP | 437 |
| | | CDRL1 | SSDVGGYNY | 447 |
| | | CDRL2 | DV | — |
| | | CDRL3 | SSYTTSTWV | 440 |
| | Chothia | CDRH1 | GGSITSISH | 448 |
| | | CDRH2 | YYSGT | 449 |
| | | CDRH3 | HDVSRFDP | 443 |
| | | CDRL1 | TGTSSDVGGYNYVS | 438 |
| | | CDRL2 | DVTNRPS | 444 |
| | | CDRL3 | SSYTTSTWV | 440 |
| | Contact | CDRH1 | TSISHYWG | 450 |
| | | CDRH2 | WIGSMYYSGTTY | 451 |
| | | CDRH3 | ARHDVSRFD | 452 |
| | | CDRL1 | VGGYNYVSWY | 289 |
| | | CDRL2 | LMIYDVTNRP | 453 |
| | | CDRL3 | SSYTTSTW | 454 |
| Z249-04 | North | CDRH1 | VASGFTFSNFDMH | 455 |
| | | CDRH2 | VISYDESKRQ | 456 |
| | | CDRH3 | ATESTQYFGALDT | 457 |
| | | CDRL1 | SGDGLADQYVY | 458 |
| | | CDRL2 | YKDSERPS | 361 |
| | | CDRL3 | QSTDRSGSYL | 459 |
| | Kabat | CDRH1 | NFDMH | 460 |
| | | CDRH2 | VISYDESKRQHADSVKG | 461 |
| | | CDRH3 | ESTQYFGALDT | 462 |
| | | CDRL1 | SGDGLADQYVY | 458 |
| | | CDRL2 | KDSERPS | 366 |
| | | CDRL3 | QSTDRSGSYL | 459 |
| | IMGT | CDRH1 | GFTFSNFD | 463 |
| | | CDRH2 | ISYDESKR | 464 |
| | | CDRH3 | ATESTQYFGALDT | 457 |
| | | CDRL1 | GLADQY | 465 |
| | | CDRL2 | KD | — |
| | | CDRL3 | QSTDRSGSYL | 459 |
| | Chothia | CDRH1 | GFTFSNF | 466 |
| | | CDRH2 | SYDESK | 467 |
| | | CDRH3 | ESTQYFGALDT | 462 |
| | | CDRL1 | SGDGLADQYVY | 458 |
| | | CDRL2 | KDSERPS | 366 |
| | | CDRL3 | QSTDRSGSYL | 459 |
| | Contact | CDRH1 | SNFDMH | 468 |
| | | CDRH2 | WVAVISYDESKRQ | 469 |
| | | CDRH3 | ATESTQYFGALD | 470 |
| | | CDRL1 | DQYVYWY | 471 |
| | | CDRL2 | LVMYKDSERP | 472 |
| | | CDRL3 | QSTDRSGSY | 473 |
| Z249-05 | North | CDRH1 | AASGFNFNFYTMH | 474 |
| | | CDRH2 | LVFYDGSKTY | 475 |
| | | CDRH3 | VRDRTTGHDYGMDV | 476 |
| | | CDRL1 | SGAGLANLYTY | 477 |
| | | CDRL2 | YKDTERPS | 61 |
| | | CDRL3 | QSADSSRSSVI | 478 |
| | Kabat | CDRH1 | FYTMH | 479 |
| | | CDRH2 | LVFYDGSKTYYADSVKG | 480 |
| | | CDRH3 | DRTTGHDYGMDV | 481 |
| | | CDRL1 | SGAGLANLYTY | 477 |
| | | CDRL2 | KDTERPS | 66 |
| | | CDRL3 | QSADSSRSSVI | 478 |
| | IMGT | CDRH1 | GFNFNFYT | 482 |
| | | CDRH2 | VFYDGSKT | 483 |
| | | CDRH3 | VRDRTTGHDYGMDV | 476 |
| | | CDRL1 | GLANLY | 484 |
| | | CDRL2 | KD | — |
| | | CDRL3 | QSADSSRSSVI | 478 |
| | Chothia | CDRH1 | GFNFNFY | 485 |
| | | CDRH2 | FYDGSK | 486 |
| | | CDRH3 | DRTTGHDYGMDV | 481 |
| | | CDRL1 | SGAGLANLYTY | 477 |
| | | CDRL2 | KDTERPS | 66 |
| | | CDRL3 | QSADSSRSSVI | 478 |
| | Contact | CDRH1 | NFYTMH | 487 |
| | | CDRH2 | WVALVFYDGSKTY | 488 |
| | | CDRH3 | VRDRTTGHDYGMD | 489 |
| | | CDRL1 | NLYTYWY | 490 |
| | | CDRL2 | LLIYKDTERP | 491 |
| | | CDRL3 | QSADSSRSSV | 492 |
| Z249-06 | North | CDRH1 | KTSGGFFSSYRIN | 414 |
| | | CDRH2 | GLVPIFETAK | 415 |
| | | CDRH3 | ARGSGSGLTYFQT | 416 |
| | | CDRL1 | RASQSITTYYLA | 417 |
| | | CDRL2 | YGASSRAT | 418 |
| | | CDRL3 | QQYDSSPT | 419 |
| | Kabat | CDRH1 | SYRIN | 420 |
| | | CDRH2 | GLVPIFETAKYAQKFQG | 421 |
| | | CDRH3 | GSGSGLTYFQT | 422 |
| | | CDRL1 | RASQSITTYYLA | 417 |
| | | CDRL2 | GASSRAT | 423 |
| | | CDRL3 | QQYDSSPT | 419 |
| | IMGT | CDRH1 | GGFFSSYR | 424 |
| | | CDRH2 | LVPIFETA | 425 |
| | | CDRH3 | ARGSGSGLTYFQT | 416 |
| | | CDRL1 | QSITTYY | 426 |
| | | CDRL2 | GA | — |
| | | CDRL3 | QQYDSSPT | 419 |
| | Chothia | CDRH1 | GGFFSSY | 427 |
| | | CDRH2 | VPIFET | 428 |
| | | CDRH3 | GSGSGLTYFQT | 422 |
| | | CDRL1 | RASQSITTYYLA | 417 |
| | | CDRL2 | GASSRAT | 423 |
| | | CDRL3 | QQYDSSPT | 419 |
| | Contact | CDRH1 | SSYRIN | 429 |
| | | CDRH2 | WMGGLVPIFETAK | 430 |
| | | CDRH3 | ARGSGSGLTYFQ | 431 |
| | | CDRL1 | TTYYLAWY | 432 |
| | | CDRL2 | LLIYGASSRA | 433 |
| | | CDRL3 | QQYDSSP | 434 |
| Z308-03 | North | CDRH1 | TFSGFSLSTGGVCIN | 493 |
| | | CDRH2 | RIDWDNDKY | 494 |
| | | CDRH3 | ARMTGLYSDNWKGFDI | 495 |
| | | CDRL1 | RASQSVDSTYLA | 496 |
| | | CDRL2 | YSASRRAT | 497 |
| | | CDRL3 | QQYGSAPGYT | 498 |
| | Kabat | CDRH1 | TGGVCIN | 499 |
| | | CDRH2 | RIDWDNDKYYRPSLKT | 500 |
| | | CDRH3 | MTGLYSDNWKGFDI | 501 |
| | | CDRL1 | RASQSVDSTYLA | 496 |
| | | CDRL2 | SASRRAT | 502 |
| | | CDRL3 | QQYGSAPGYT | 498 |
| | IMGT | CDRH1 | GFSLSTGGVC | 503 |
| | | CDRH2 | IDWDNDK | 504 |
| | | CDRH3 | ARMTGLYSDNWKGFDI | 495 |
| | | CDRL1 | QSVDSTY | 505 |
| | | CDRL2 | SA | — |
| | | CDRL3 | QQYGSAPGYT | 498 |
| | Chothia | CDRH1 | GFSLSTGGV | 506 |
| | | CDRH2 | DWDND | 507 |
| | | CDRH3 | MTGLYSDNWKGFDI | 501 |
| | | CDRL1 | RASQSVDSTYLA | 496 |
| | | CDRL2 | SASRRAT | 502 |
| | | CDRL3 | QQYGSAPGYT | 498 |
| | Contact | CDRH1 | STGGVCIN | 508 |
| | | CDRH2 | WLARIDWDNDKY | 509 |
| | | CDRH3 | ARMTGLYSDNWKGFD | 510 |
| | | CDRL1 | DSTYLAWY | 511 |
| | | CDRL2 | LLIYSASRRA | 512 |
| | | CDRL3 | QQYGSAPGY | 513 |
| Z308-04 | North | CDRH1 | KVSGYRFTSYWIG | 514 |
| | | CDRH2 | VIYPPDSDTA | 515 |
| | | CDRH3 | ARQGRGFDY | 516 |
| | | CDRL1 | RTSQNIYTYLN | 517 |
| | | CDRL2 | NTASSLQG | 518 |
| | | CDRL3 | QQSYDSPRT | 519 |
| | Kabat | CDRH1 | SYWIG | 520 |
| | | CDRH2 | VIYPPDSDTAYSPSFQG | 521 |
| | | CDRH3 | QGRGFDY | 522 |
| | | CDRL1 | RTSQNIYTYLN | 517 |
| | | CDRL2 | TASSLQG | 523 |
| | | CDRL3 | QQSYDSPRT | 519 |
| | IMGT | CDRH1 | GYRFTSYW | 524 |
| | | CDRH2 | IYPPDSDT | 525 |
| | | CDRH3 | ARQGRGFDY | 516 |
| | | CDRL1 | QNIYTY | 526 |
| | | CDRL2 | TA | — |
| | | CDRL3 | QQSYDSPRT | 519 |
| | Chothia | CDRH1 | GYRFTSY | 527 |
| | | CDRH2 | YPPDSD | 528 |
| | | CDRH3 | QGRGFDY | 522 |
| | | CDRL1 | RTSQNIYTYLN | 517 |
| | | CDRL2 | TASSLQG | 523 |
| | | CDRL3 | QQSYDSPRT | 519 |
| | Contact | CDRH1 | TSYWIG | 529 |
| | | CDRH2 | WMGVIYPPDSDTA | 530 |
| | | CDRH3 | ARQGRGFD | 531 |
| | | CDRL1 | YTYLNWY | 532 |
| | | CDRL2 | LLINTASSLQ | 533 |
| | | CDRL3 | QQSYDSPR | 534 |
| Z308-05 | North | CDRH1 | TVSGFSLNTAEVCWN | 535 |
| | | CDRH2 | RIDWDDDKY | 272 |
| | | CDRH3 | ARMTGLYTDNWKGFDM | 536 |
| | | CDRL1 | RASQTIDSTYFA | 537 |
| | | CDRL2 | YSASRRAT | 497 |
| | | CDRL3 | QQYGHAPGYT | 538 |
| | Kabat | CDRH1 | TAEVCWN | 539 |
| | | CDRH2 | RIDWDDDKYIRPSLKT | 540 |
| | | CDRH3 | MTGLYTDNWKGFDM | 541 |
| | | CDRL1 | RASQTIDSTYFA | 537 |
| | | CDRL2 | SASRRAT | 502 |
| | | CDRL3 | QQYGHAPGYT | 538 |
| | IMGT | CDRH1 | GFSLNTAEVC | 542 |
| | | CDRH2 | IDWDDDK | 282 |
| | | CDRH3 | ARMTGLYTDNWKGFDM | 536 |
| | | CDRL1 | QTIDSTY | 542 |
| | | CDRL2 | SA | — |
| | | CDRL3 | QQYGHAPGYT | 538 |
| | Chothia | CDRH1 | GFSLNTAEV | 543 |
| | | CDRH2 | DWDDD | 285 |
| | | CDRH3 | MTGLYTDNWKGFDM | 541 |
| | | CDRL1 | RASQTIDSTYFA | 537 |
| | | CDRL2 | SASRRAT | 502 |
| | | CDRL3 | QQYGHAPGYT | 538 |
| | Contact | CDRH1 | NTAEVCWN | 544 |
| | | CDRH2 | WLARIDWDDDKY | 287 |
| | | CDRH3 | ARMTGLYTDNWKGFD | 545 |
| | | CDRL1 | DSTYFAWY | 546 |
| | | CDRL2 | LLIYSASRRA | 512 |
| | | CDRL3 | QQYGHAPGY | 547 |
| Z504-03 | North | CDRH1 | KAPGGTFSRYAIS | 548 |
| | | CDRH2 | GIIPMFDITN | 549 |
| | | CDRH3 | ATESCSGGNCYWDHDAFDI | 550 |
| | | CDRL1 | RASQSLSSSYLA | 551 |
| | | CDRL2 | YGASTRAT | 552 |
| | | CDRL3 | QQYGRSLPRT | 1308 |
| | Kabat | CDRH1 | RYAIS | 553 |
| | | CDRH2 | GIIPMFDITNYAQKLQD | 554 |
| | | CDRH3 | ESCSGGNCYWDHDAFDI | 555 |
| | | CDRL1 | RASQSLSSSYLA | 551 |
| | | CDRL2 | GASTRAT | 556 |
| | | CDRL3 | QQYGRSLPRT | 1308 |
| | IMGT | CDRH1 | GGTFSRYA | 557 |
| | | CDRH2 | IIPMFDIT | 558 |
| | | CDRH3 | ATESCSGGNCYWDHDAFDI | 550 |
| | | CDRL1 | QSLSSSY | 559 |
| | | CDRL2 | GA | — |
| | | CDRL3 | QQYGRSLPRT | 1308 |
| | Chothia | CDRH1 | GGTFSRY | 560 |
| | | CDRH2 | IPMFDI | 561 |
| | | CDRH3 | ESCSGGNCYWDHDAFDI | 555 |
| | | CDRL1 | RASQSLSSSYLA | 551 |
| | | CDRL2 | GASTRAT | 556 |
| | | CDRL3 | QQYGRSLPRT | 1308 |
| | Contact | CDRH1 | SRYAIS | 562 |
| | | CDRH2 | WMGGIIPMFDITN | 563 |
| | | CDRH3 | ATESCSGGNCYWDHDAFD | 564 |
| | | CDRL1 | SSSYLAWY | 565 |
| | | CDRL2 | LLIYGASTRA | 566 |
| | | CDRL3 | QQYGRSLPR | 567 |
| Z504-04 | North | CDRH1 | KASGGTFSSYAIS | 568 |
| | | CDRH2 | GIIPIFDIAN | 569 |
| | | CDRH3 | ASENCGGGNCYWEHDAFDI | 570 |
| | | CDRL1 | RASQSVSGGDLA | 571 |
| | | CDRL2 | YGASIRAT | 572 |
| | | CDRL3 | QHYGNSPPRT | 573 |
| | Kabat | CDRH1 | SYAIS | 342 |
| | | CDRH2 | GIIPIFDIANYAQKFQG | 574 |
| | | CDRH3 | ENCGGGNCYWEHDAFDI | 570 |
| | | CDRL1 | RASQSVSGGDLA | 571 |
| | | CDRL2 | GASIRAT | 575 |
| | | CDRL3 | QHYGNSPPRT | 573 |
| | IMGT | CDRH1 | GGTFSSYA | 576 |
| | | CDRH2 | IIPIFDIA | 577 |
| | | CDRH3 | ASENCGGGNCYWEHDAFDI | 570 |
| | | CDRL1 | QSVSGGD | 578 |
| | | CDRL2 | GA | — |
| | | CDRL3 | QHYGNSPPRT | 573 |
| | Chothia | CDRH1 | GGTFSSY | 579 |
| | | CDRH2 | IPIFDI | 580 |
| | | CDRH3 | ENCGGGNCYWEHDAFDI | 570 |
| | | CDRL1 | RASQSVSGGDLA | 571 |
| | | CDRL2 | GASIRAT | 575 |
| | | CDRL3 | QHYGNSPPRT | 573 |
| | Contact | CDRH1 | SSYAIS | 351 |
| | | CDRH2 | WMGGIIPIFDIAN | 581 |
| | | CDRH3 | ASENCGGGNCYWEHDAFD | 582 |
| | | CDRL1 | SGGDLAWY | 583 |
| | | CDRL2 | LLMYGASIRA | 584 |
| | | CDRL3 | QHYGNSPPR | 585 |
| Z504-06 | North | CDRH1 | KAYGGTFSTYAIS | 586 |
| | | CDRH2 | GIIPIFDIAN | 569 |
| | | CDRH3 | ASENCSGGNCYWEHDAFDI | 587 |
| | | CDRL1 | RASQSVSGGDLA | 571 |
| | | CDRL2 | YGASIRAT | 572 |
| | | CDRL3 | QQYGNSPPRT | 588 |
| | Kabat | CDRH1 | TYAIS | 589 |
| | | CDRH2 | GIIPIFDIANYAQKFQG | 574 |
| | | CDRH3 | ENCSGGNCYWEHDAFDI | 590 |
| | | CDRL1 | RASQSVSGGDLA | 571 |
| | | CDRL2 | GASIRAT | 575 |
| | | CDRL3 | QQYGNSPPRT | 588 |
| | IMGT | CDRH1 | GGTFSTYA | 591 |
| | | CDRH2 | IIPIFDIA | 577 |
| | | CDRH3 | ASENCSGGNCYWEHDAFDI | 587 |
| | | CDRL1 | QSVSGGD | 578 |
| | | CDRL2 | GA | — |
| | | CDRL3 | QQYGNSPPRT | 588 |
| | Chothia | CDRH1 | GGTFSTY | 592 |
| | | CDRH2 | IPIFDI | 580 |
| | | CDRH3 | ENCSGGNCYWEHDAFDI | 590 |
| | | CDRL1 | RASQSVSGGDLA | 571 |
| | | CDRL2 | GASIRAT | 575 |
| | | CDRL3 | QQYGNSPPRT | 588 |
| | Contact | CDRH1 | STYAIS | 593 |
| | | CDRH2 | WMGGIIPIFDIAN | 581 |
| | | CDRH3 | ASENCSGGNCYWEHDAFD | 594 |
| | | CDRL1 | SGGDLAWY | 583 |
| | | CDRL2 | LLMYGASIRA | 584 |
| | | CDRL3 | QQYGNSPPR | 595 |
| Z504-07 | North | CDRH1 | QTSGYSFASFWIG | 596 |
| | | CDRH2 | IIYPSDSDTT | 597 |
| | | CDRH3 | AKHGVGAAIDH | 598 |
| | | CDRL1 | RTSQVISGYLN | 599 |
| | | CDRL2 | YTTSTLQP | 600 |
| | | CDRL3 | QQSYSSPRT | 601 |
| | Kabat | CDRH1 | SFWIG | 602 |
| | | CDRH2 | IIYPSDSDTTYSPTFQG | 603 |
| | | CDRH3 | HGVGAAIDH | 604 |
| | | CDRL1 | RTSQVISGYLN | 599 |
| | | CDRL2 | TTSTLQP | 605 |
| | | CDRL3 | QQSYSSPRT | 601 |
| | IMGT | CDRH1 | GYSFASFW | 606 |
| | | CDRH2 | IYPSDSDT | 607 |
| | | CDRH3 | HGVGAAIDH | 604 |
| | | CDRL1 | QVISGY | 608 |
| | | CDRL2 | IT | — |
| | | CDRL3 | QQSYSSPRT | 601 |
| | Chothia | CDRH1 | GYSFASF | 609 |
| | | CDRH2 | YPSDSD | 610 |
| | | CDRH3 | HGVGAAIDH | 604 |
| | | CDRL1 | RTSQVISGYLN | 599 |
| | | CDRL2 | TTSTLQP | 605 |
| | | CDRL3 | QQSYSSPRT | 601 |
| | Contact | CDRH1 | ASFWIG | 611 |
| | | CDRH2 | WMGIIYPSDSDTT | 612 |
| | | CDRH3 | HGVGAAID | 613 |
| | | CDRL1 | SGYLNWY | 614 |
| | | CDRL2 | LLIYTTSTLQ | 615 |
| | | CDRL3 | QQSYSSPR | 616 |
| Z513-01 | North | CDRH1 | TVSGGSIGGGPYHWS | 617 |
| | | CDRH2 | HVETSGDTK | 618 |
| | | CDRH3 | ARGTAMDAFDI | 619 |
| | | CDRL1 | RASQDISNWLA | 620 |
| | | CDRL2 | YVASGLQS | 621 |
| | | CDRL3 | QQGNSFPLT | 622 |
| | Kabat | CDRH1 | GGPYHWS | 623 |
| | | CDRH2 | HVETSGDTKTNPSFRS | 624 |
| | | CDRH3 | GTAMDAFDI | 625 |
| | | CDRL1 | RASQDISNWLA | 620 |
| | | CDRL2 | VASGLQS | 626 |
| | | CDRL3 | QQGNSFPLT | 622 |
| | IMGT | CDRH1 | GGSIGGGPYH | 627 |
| | | CDRH2 | VETSGDT | 628 |
| | | CDRH3 | GTAMDAFDI | 625 |
| | | CDRL1 | QDISNW | 629 |
| | | CDRL2 | VA | — |
| | | CDRL3 | QQGNSFPLT | 622 |
| | Chothia | CDRH1 | GGSIGGGPY | 630 |
| | | CDRH2 | ETSGD | 631 |
| | | CDRH3 | GTAMDAFDI | 625 |
| | | CDRL1 | RASQDISNWLA | 620 |
| | | CDRL2 | VASGLQS | 626 |
| | | CDRL3 | QQGNSFPLT | 622 |
| | Contact | CDRH1 | GGGPYHWS | 632 |
| | | CDRH2 | WIGHVETSGDTK | 633 |
| | | CDRH3 | GTAMDAFD | 634 |
| | | CDRL1 | SNWLAWY | 635 |
| | | CDRL2 | LLIYVASGLQ | 636 |
| | | CDRL3 | QQGNSFPL | 637 |
| Z513-02 | North | CDRH1 | AASGFSFSGYGMH | 638 |
| | | CDRH2 | FIWYDGSSKK | 639 |
| | | CDRH3 | ARDVLGELPD | 640 |
| | | CDRL1 | RASQGISNSLG | 641 |
| | | CDRL2 | YAASSLQS | 642 |
| | | CDRL3 | QQYNTYPLT | 643 |
| | Kabat | CDRH1 | GYGMH | 644 |
| | | CDRH2 | FIWYDGSSKKYGDSVKG | 645 |
| | | CDRH3 | DVLGELPD | 646 |
| | | CDRL1 | RASQGISNSLG | 641 |
| | | CDRL2 | AASSLQS | 647 |
| | | CDRL3 | QQYNTYPLT | 643 |
| | IMGT | CDRH1 | GFSFSGYG | 648 |
| | | CDRH2 | IWYDGSSK | 649 |
| | | CDRH3 | ARDVLGELPD | 640 |
| | | CDRL1 | QGISNS | 650 |
| | | CDRL2 | AA | — |
| | | CDRL3 | QQYNTYPLT | 643 |
| | Chothia | CDRH1 | GFSFSGY | 651 |
| | | CDRH2 | WYDGSS | 652 |
| | | CDRH3 | DVLGELPD | 646 |
| | | CDRL1 | RASQGISNSLG | 641 |
| | | CDRL2 | AASSLQS | 647 |
| | | CDRL3 | QQYNTYPLT | 643 |
| | Contact | CDRH1 | SGYGMH | 653 |
| | | CDRH2 | WVAFIWYDGSSKK | 654 |
| | | CDRH3 | ARDVLGELP | 655 |
| | | CDRL1 | SNSLGWF | 656 |
| | | CDRL2 | SLIYAASSLQ | 657 |
| | | CDRL3 | QQYNTYPL | 658 |
| Z538-03 | North | CDRH1 | ATSGFTFSNYGLH | 659 |
| | | CDRH2 | AIWFDGRTF | 660 |
| | | CDRH3 | ARETWSYAGGVFDY | 661 |
| | | CDRL1 | TLSSGHSGHAIA | 662 |
| | | CDRL2 | KVHSDGSHNKGD | 663 |
| | | CDRL3 | QTWDTGLWV | 664 |
| | Kabat | CDRH1 | NYGLH | 665 |
| | | CDRH2 | AIWFDGRTFYADSVKD | 666 |
| | | CDRH3 | ETWSYAGGVFDY | 667 |
| | | CDRL1 | TLSSGHSGHAIA | 662 |
| | | CDRL2 | VHSDGSHNKGD | 668 |
| | | CDRL3 | QTWDTGLWV | 664 |
| | IMGT | CDRH1 | GFTFSNYG | 669 |
| | | CDRH2 | IWFDGRT | 670 |
| | | CDRH3 | ARETWSYAGGVFDY | 661 |
| | | CDRL1 | SGHSGHA | 671 |
| | | CDRL2 | VHSDGS | 672 |
| | | CDRL3 | QTWDTGLWV | 664 |
| | Chothia | CDRH1 | GFTFSNY | 673 |
| | | CDRH2 | WFDGR | 674 |
| | | CDRH3 | ETWSYAGGVFDY | 667 |
| | | CDRL1 | TLSSGHSGHAIA | 662 |
| | | CDRL2 | VHSDGSHNKGD | 668 |
| | | CDRL3 | QTWDTGLWV | 664 |
| | Contact | CDRH1 | SNYGLH | 675 |
| | | CDRH2 | WLAAIWFDGRTF | 676 |
| | | CDRH3 | ARETWSYAGGVFD | 677 |
| | | CDRL1 | SGHAIAWH | 678 |
| | | CDRL2 | FLMKVHSDGSHNKG | 679 |
| | | CDRL3 | QTWDTGLW | 680 |
| Z573-03 | North | CDRH1 | KASGYTFTTYYIH | 681 |
| | | CDRH2 | VLNPSRNRTI | 682 |
| | | CDRH3 | ARDAGLGPYYYYMDV | 683 |
| | | CDRL1 | SGDGLTKQYAF | 684 |
| | | CDRL2 | YKDSERPS | 361 |
| | | CDRL3 | QSADRTDIYVV | 685 |
| | Kabat | CDRH1 | TYYIH | 686 |
| | | CDRH2 | VLNPSRNRTIYAQKFQG | 687 |
| | | CDRH3 | DAGLGPYYYYMDV | 688 |
| | | CDRL1 | SGDGLTKQYAF | 684 |
| | | CDRL2 | KDSERPS | 366 |
| | | CDRL3 | QSADRTDIYVV | 685 |
| | IMGT | CDRH1 | GYTFTTYY | 689 |
| | | CDRH2 | LNPSRNRT | 690 |
| | | CDRH3 | ARDAGLGPYYYYMDV | 683 |
| | | CDRL1 | GLTKQY | 691 |
| | | CDRL2 | KD | — |
| | | CDRL3 | QSADRTDIYVV | 685 |
| | Chothia | CDRH1 | GYTFTTY | 692 |
| | | CDRH2 | NPSRNR | 693 |
| | | CDRH3 | DAGLGPYYYYMDV | 688 |
| | | CDRL1 | SGDGLTKQYAF | 684 |
| | | CDRL2 | KDSERPS | 366 |
| | | CDRL3 | QSADRTDIYVV | 685 |
| | Contact | CDRH1 | TTYYIH | 694 |
| | | CDRH2 | WVGVLNPSRNRTI | 695 |
| | | CDRH3 | ARDAGLGPYYYYMD | 696 |
| | | CDRL1 | KQYAFWY | 697 |
| | | CDRL2 | LVIYKDSERP | 375 |
| | | CDRL3 | QSADRTDIYV | 698 |
| Z582-03 | North | CDRH1 | KTSGGTFSSATLS | 699 |
| | | CDRH2 | GITPMFTTAN | 700 |
| | | CDRH3 | ARQFGHESRDSYYLY | 701 |
| | | CDRL1 | RASQTLSSSYLA | 702 |
| | | CDRL2 | YGASSRVA | 703 |
| | | CDRL3 | QQYGSSRT | 704 |
| | Kabat | CDRH1 | SATLS | 705 |
| | | CDRH2 | GITPMFTTANYAQKFQG | 706 |
| | | CDRH3 | QFGHESRDSYYLY | 707 |
| | | CDRL1 | RASQTLSSSYLA | 702 |
| | | CDRL2 | GASSRVA | 708 |
| | | CDRL3 | QQYGSSRT | 704 |
| | IMGT | CDRH1 | GGTFSSAT | 709 |
| | | CDRH2 | ITPMFTTA | 710 |
| | | CDRH3 | ARQFGHESRDSYYLY | 701 |
| | | CDRL1 | QTLSSSY | 711 |
| | | CDRL2 | GA | — |
| | | CDRL3 | QQYGSSRT | 704 |
| | Chothia | CDRH1 | GGTFSSA | 712 |
| | | CDRH2 | TPMFTT | 713 |
| | | CDRH3 | QFGHESRDSYYLY | 707 |
| | | CDRL1 | RASQTLSSSYLA | 702 |
| | | CDRL2 | GASSRVA | 708 |
| | | CDRL3 | QQYGSSRT | 704 |
| | Contact | CDRH1 | SSATLS | 714 |
| | | CDRH2 | WMGGITPMFTTAN | 715 |
| | | CDRH3 | ARQFGHESRDSYYL | 716 |
| | | CDRL1 | SSSYLAWY | 565 |
| | | CDRL2 | LLIYGASSRV | 717 |
| | | CDRL3 | QQYGSSR | 718 |
| Z582-14 | North | CDRH1 | TVSGYSISSGYYWG | 719 |
| | | CDRH2 | SIYHSGSTY | 720 |
| | | CDRH3 | ARSARAVVMDAFHI | 721 |
| | | CDRL1 | TGSSGSIASNYVQ | 722 |
| | | CDRL2 | YEDNQRPS | 723 |
| | | CDRL3 | QSYDNIHPWV | 724 |
| | Kabat | CDRH1 | SGYYWG | 725 |
| | | CDRH2 | SIYHSGSTYYNPSLNS | 726 |
| | | CDRH3 | SARAVVMDAFHI | 727 |
| | | CDRL1 | TGSSGSIASNYVQ | 722 |
| | | CDRL2 | EDNQRPS | 728 |
| | | CDRL3 | QSYDNIHPWV | 724 |
| | IMGT | CDRH1 | GYSISSGYY | 729 |
| | | CDRH2 | IYHSGST | 730 |
| | | CDRH3 | ARSARAVVMDAFHI | 721 |
| | | CDRL1 | SGSIASNY | 731 |
| | | CDRL2 | ED | — |
| | | CDRL3 | QSYDNIHPWV | 724 |
| | Chothia | CDRH1 | GYSISSGY | 732 |
| | | CDRH2 | YHSGS | 733 |
| | | CDRH3 | SARAVVMDAFHI | 727 |
| | | CDRL1 | TGSSGSIASNYVQ | 722 |
| | | CDRL2 | EDNQRPS | 728 |
| | | CDRL3 | QSYDNIHPWV | 724 |
| | Contact | CDRH1 | SSGYYWG | 734 |
| | | CDRH2 | WIGSIYHSGSTY | 735 |
| | | CDRH3 | ARSARAVVMDAFH | 736 |
| | | CDRL1 | IASNYVQWY | 737 |
| | | CDRL2 | TVIYEDNQRP | 738 |
| | | CDRL3 | QSYDNIHPW | 739 |
| Z582-15 | North | CDRH1 | TVSGYSISSGYYWG | 719 |
| | | CDRH2 | SIYHSGSTY | 720 |
| | | CDRH3 | ARSARAVVMDAFHI | 721 |
| | | CDRL1 | GGNNIGSKSVH | 740 |
| | | CDRL2 | YYDSDRPS | 741 |
| | | CDRL3 | QVWDGSTDHFYV | 742 |
| | Kabat | CDRH1 | SGYYWG | 725 |
| | | CDRH2 | SIYHSGSTYYNPSLNS | 726 |
| | | CDRH3 | SARAVVMDAFHI | 727 |
| | | CDRL1 | GGNNIGSKSVH | 740 |
| | | CDRL2 | YDSDRPS | 743 |
| | | CDRL3 | QVWDGSTDHFYV | 742 |
| | IMGT | CDRH1 | GYSISSGYY | 729 |
| | | CDRH2 | IYHSGST | 730 |
| | | CDRH3 | ARSARAVVMDAFHI | 721 |
| | | CDRL1 | NIGSKS | 744 |
| | | CDRL2 | YD | — |
| | | CDRL3 | QVWDGSTDHFYV | 742 |
| | Chothia | CDRH1 | GYSISSGY | 732 |
| | | CDRH2 | YHSGS | 733 |
| | | CDRH3 | SARAVVMDAFHI | 727 |
| | | CDRL1 | GGNNIGSKSVH | 740 |
| | | CDRL2 | YDSDRPS | 743 |
| | | CDRL3 | QVWDGSTDHFYV | 742 |
| | Contact | CDRH1 | SSGYYWG | 734 |
| | | CDRH2 | WIGSIYHSGSTY | 735 |
| | | CDRH3 | ARSARAVVMDAFH | 736 |
| | | CDRL1 | SKSVHWY | 745 |
| | | CDRL2 | LVIYYDSDRP | 746 |
| | | CDRL3 | QVWDGSTDHFY | 747 |
| Z082-U1 | North | CDRH1 | TVSGGSISSGEYYWS | 748 |
| | | CDRH2 | YISYAGNTY | 749 |
| | | CDRH3 | ARDFVGTNWYDRYAFHI | 750 |
| | | CDRL1 | TGTSSDVGGYHYVS | 751 |
| | | CDRL2 | YDVTKRPS | 752 |
| | | CDRL3 | CSYAGSYAFWV | 753 |
| | Kabat | CDRH1 | SGEYYWS | 754 |
| | | CDRH2 | YISYAGNTYYNPSLKS | 755 |
| | | CDRH3 | DFVGTNWYDRYAFHI | 756 |
| | | CDRL1 | TGTSSDVGGYHYVS | 751 |
| | | CDRL2 | DVTKRPS | 757 |
| | | CDRL3 | CSYAGSYAFWV | 753 |
| | IMGT | CDRH1 | GGSISSGEYY | 758 |
| | | CDRH2 | ISYAGNT | 759 |
| | | CDRH3 | ARDFVGTNWYDRYAFHI | 750 |
| | | CDRL1 | SSDVGGYHY | 760 |
| | | CDRL2 | DV | — |
| | | CDRL3 | CSYAGSYAFWV | 753 |
| | Chothia | CDRH1 | GGSISSGEY | 761 |
| | | CDRH2 | SYAGN | 762 |
| | | CDRH3 | DFVGTNWYDRYAFHI | 756 |
| | | CDRL1 | TGTSSDVGGYHYVS | 751 |
| | | CDRL2 | DVTKRPS | 757 |
| | | CDRL3 | CSYAGSYAFWV | 753 |
| | Contact | CDRH1 | SSGEYYWS | 763 |
| | | CDRH2 | WIGYISYAGNTY | 764 |
| | | CDRH3 | ARDFVGTNWYDRYAFH | 765 |
| | | CDRL1 | VGGYHYVSWY | 766 |
| | | CDRL2 | LILYDVTKRP | 767 |
| | | CDRL3 | CSYAGSYAFW | 768 |
| Z082-U5 | North | CDRH1 | GASGFNFIDYAMY | 769 |
| | | CDRH2 | SITNDAKSTF | 770 |
| | | CDRH3 | ARGRSHYYDSSGFDY | 771 |
| | | CDRL1 | GGDSLGSKSVH | 772 |
| | | CDRL2 | FDDSDRPS | 773 |
| | | CDRL3 | QVWDAGALWV | 774 |
| | Kabat | CDRH1 | DYAMY | 775 |
| | | CDRH2 | SITNDAKSTFYGNSVKG | 776 |
| | | CDRH3 | GRSHYYDSSGFDY | 777 |
| | | CDRL1 | GGDSLGSKSVH | 772 |
| | | CDRL2 | DDSDRPS | 778 |
| | | CDRL3 | QVWDAGALWV | 774 |
| | IMGT | CDRH1 | GFNFIDYA | 779 |
| | | CDRH2 | ITNDAKST | 780 |
| | | CDRH3 | ARGRSHYYDSSGFDY | 771 |
| | | CDRL1 | SLGSKS | 781 |
| | | CDRL2 | DD | — |
| | | CDRL3 | QVWDAGALWV | 774 |
| | Chothia | CDRH1 | GFNFIDY | 782 |
| | | CDRH2 | TNDAKS | 783 |
| | | CDRH3 | GRSHYYDSSGFDY | 777 |
| | | CDRL1 | GGDSLGSKSVH | 772 |
| | | CDRL2 | DDSDRPS | 778 |
| | | CDRL3 | QVWDAGALWV | 774 |
| | Contact | CDRH1 | IDYAMY | 784 |
| | | CDRH2 | YVSSITNDAKSTF | 785 |
| | | CDRH3 | ARGRSHYYDSSGFD | 786 |
| | | CDRL1 | SKSVHWY | 745 |
| | | CDRL2 | LVVFDDSDRP | 787 |
| | | CDRL3 | QVWDAGALW | 788 |
| Z082-U6 | North | CDRH1 | SVSGDSISTTSYYWD | 789 |
| | | CDRH2 | SINYSGNTY | 790 |
| | | CDRH3 | ARRGVPAGMVPSWIDP | 791 |
| | | CDRL1 | QASEDIRNYLN | 792 |
| | | CDRL2 | YDASNLKT | 793 |
| | | CDRL3 | QQYDTVPLT | 794 |
| | Kabat | CDRH1 | TTSYYWD | 795 |
| | | CDRH2 | SINYSGNTYYSPLLKS | 796 |
| | | CDRH3 | RGVPAGMVPSWIDP | 797 |
| | | CDRL1 | QASEDIRNYLN | 792 |
| | | CDRL2 | DASNLKT | 798 |
| | | CDRL3 | QQYDTVPLT | 794 |
| | IMGT | CDRH1 | GDSISTTSYY | 799 |
| | | CDRH2 | INYSGNT | 800 |
| | | CDRH3 | ARRGVPAGMVPSWIDP | 791 |
| | | CDRL1 | EDIRNY | 801 |
| | | CDRL2 | DA | — |
| | | CDRL3 | QQYDTVPLT | 794 |
| | Chothia | CDRH1 | GDSISTTSY | 802 |
| | | CDRH2 | NYSGN | 803 |
| | | CDRH3 | RGVPAGMVPSWIDP | 797 |
| | | CDRL1 | QASEDIRNYLN | 792 |
| | | CDRL2 | DASNLKT | 798 |
| | | CDRL3 | QQYDTVPLT | 794 |
| | Contact | CDRH1 | STTSYYWD | 804 |
| | | CDRH2 | WIGSINYSGNTY | 805 |
| | | CDRH3 | ARRGVPAGMVPSWID | 806 |
| | | CDRL1 | RNYLNWY | 807 |
| | | CDRL2 | VLIYDASNLK | 808 |
| | | CDRL3 | QQYDTVPL | 809 |
| W056-01 | North | CDRH1 | KGSGYHFPTYWIA | 810 |
| | | CDRH2 | VIYPRDSDTK | 811 |
| | | CDRH3 | ARATGDY | 315 |
| | | CDRL1 | RAGENVSTWLT | 812 |
| | | CDRL2 | YKASTLET | 813 |
| | | CDRL3 | QQYKSYSRT | 814 |
| | Kabat | CDRH1 | TYWIA | 319 |
| | | CDRH2 | VIYPRDSDTKYSPSFQG | 815 |
| | | CDRH3 | ATGDY | 321 |
| | | CDRL1 | RAGENVSTWLT | 812 |
| | | CDRL2 | KASTLET | 816 |
| | | CDRL3 | QQYKSYSRT | 814 |
| | IMGT | CDRH1 | GYHFPTYW | 817 |
| | | CDRH2 | IYPRDSDT | 818 |
| | | CDRH3 | ARATGDY | 315 |
| | | CDRL1 | ENVSTW | 819 |
| | | CDRL2 | KA | — |
| | | CDRL3 | QQYKSYSRT | 814 |
| | Chothia | CDRH1 | GYHFPTY | 820 |
| | | CDRH2 | YPRDSD | 821 |
| | | CDRH3 | ATGDY | 321 |
| | | CDRL1 | RAGENVSTWLT | 812 |
| | | CDRL2 | KASTLET | 816 |
| | | CDRL3 | QQYKSYSRT | 814 |
| | Contact | CDRH1 | PTYWIA | 822 |
| | | CDRH2 | WMGVIYPRDSDTK | 823 |
| | | CDRH3 | ARATGD | 325 |
| | | CDRL1 | STWLTWY | 326 |
| | | CDRL2 | RLMYKASTLE | 824 |
| | | CDRL3 | QQYKSYSR | 825 |
| W056-02 | North | CDRH1 | QGFGYSFPTYWIA | 826 |
| | | CDRH2 | IIYPDDSDTT | 827 |
| | | CDRH3 | ARGMGDH | 828 |
| | | CDRL1 | KSSQSLLFGDGATYLY | 829 |
| | | CDRL2 | SKVSSRFS | 830 |
| | | CDRL3 | MQALHMPRT | 831 |
| | Kabat | CDRH1 | TYWIA | 319 |
| | | CDRH2 | IIYPDDSDTTYSPSFQG | 832 |
| | | CDRH3 | GMGDH | 833 |
| | | CDRL1 | KSSQSLLFGDGATYLY | 829 |
| | | CDRL2 | KVSSRFS | 834 |
| | | CDRL3 | MQALHMPRT | 831 |
| | IMGT | CDRH1 | GYSFPTYW | 835 |
| | | CDRH2 | IYPDDSDT | 324 |
| | | CDRH3 | ARGMGDH | 828 |
| | | CDRL1 | QSLLFGDGATY | 836 |
| | | CDRL2 | KV | — |
| | | CDRL3 | MQALHMPRT | 831 |
| | Chothia | CDRH1 | GYSFPTY | 837 |
| | | CDRH2 | YPDDSD | 327 |
| | | CDRH3 | GMGDH | 833 |
| | | CDRL1 | KSSQSLLFGDGATYLY | 829 |
| | | CDRL2 | KVSSRFS | 834 |
| | | CDRL3 | MQALHMPRT | 831 |
| | Contact | CDRH1 | PTYWIA | 822 |
| | | CDRH2 | WMGIIYPDDSDTT | 838 |
| | | CDRH3 | ARGMGD | 839 |
| | | CDRL1 | LFGDGATYLYWF | 840 |
| | | CDRL2 | LLISKVSSRF | 841 |
| | | CDRL3 | MQALHMPR | 842 |
| W917-01 | North | CDRH1 | AASGFTVSSNYMS | 843 |
| | | CDRH2 | VIYSGGSTY | 844 |
| | | CDRH3 | ARDRKIVVRRGGLDI | 845 |
| | | CDRL1 | RASQGISSYLA | 295 |
| | | CDRL2 | YAASTLQS | 296 |
| | | CDRL3 | QQYYSYPHT | 846 |
| | Kabat | CDRH1 | SNYMS | 847 |
| | | CDRH2 | VIYSGGSTYYADSVKG | 848 |
| | | CDRH3 | DRKIVVRRGGLDI | 849 |
| | | CDRL1 | RASQGISSYLA | 295 |
| | | CDRL2 | AASTLQS | 301 |
| | | CDRL3 | QQYYSYPHT | 846 |
| | IMGT | CDRH1 | GFTVSSNY | 850 |
| | | CDRH2 | IYSGGST | 851 |
| | | CDRH3 | ARDRKIVVRRGGLDI | 845 |
| | | CDRL1 | QGISSY | 304 |
| | | CDRL2 | AA | — |
| | | CDRL3 | QQYYSYPHT | 846 |
| | Chothia | CDRH1 | GFTVSSN | 852 |
| | | CDRH2 | YSGGS | 853 |
| | | CDRH3 | DRKIVVRRGGLDI | 849 |
| | | CDRL1 | RASQGISSYLA | 295 |
| | | CDRL2 | AASTLQS | 301 |
| | | CDRL3 | QQYYSYPHT | 846 |
| | Contact | CDRH1 | SSNYMS | 854 |
| | | CDRH2 | WVSVIYSGGSTY | 855 |
| | | CDRH3 | ARDRKIVVRRGGLD | 856 |
| | | CDRL1 | SSYLAWY | 310 |
| | | CDRL2 | LLIYAASTLQ | 857 |
| | | CDRL3 | QQYYSYPHT | 846 |
| W917-02 | North | CDRH1 | AASGFTFSSYSMN | 858 |
| | | CDRH2 | YISSSSSTIY | 859 |
| | | CDRH3 | ARDGAAAGVNP | 860 |
| | | CDRL1 | RASQSISSWLA | 861 |
| | | CDRL2 | YKASSLES | 862 |
| | | CDRL3 | QQYNSFPWT | 863 |
| | Kabat | CDRH1 | SYSMN | 864 |
| | | CDRH2 | YISSSSSTIYYADSVKG | 865 |
| | | CDRH3 | DGAAAGVNP | 866 |
| | | CDRL1 | RASQSISSWLA | 861 |
| | | CDRL2 | KASSLES | 867 |
| | | CDRL3 | QQYNSFPWT | 863 |
| | IMGT | CDRH1 | GFTFSSYS | 864 |
| | | CDRH2 | ISSSSSTI | 865 |
| | | CDRH3 | ARDGAAAGVNP | 860 |
| | | CDRL1 | QSISSW | 866 |
| | | CDRL2 | KA | — |
| | | CDRL3 | QQYNSFPWT | 863 |
| | Chothia | CDRH1 | GFTFSSY | 305 |
| | | CDRH2 | SSSSST | 867 |
| | | CDRH3 | DGAAAGVNP | 866 |
| | | CDRL1 | RASQSISSWLA | 861 |
| | | CDRL2 | KASSLES | 867 |
| | | CDRL3 | QQYNSFPWT | 863 |
| | Contact | CDRH1 | SSYSMN | 868 |
| | | CDRH2 | WVSYISSSSSTIY | 869 |
| | | CDRH3 | ARDGAAAGVN | 870 |
| | | CDRL1 | SSWLAWY | 871 |
| | | CDRL2 | LLIYKASSLE | 872 |
| | | CDRL3 | QQYNSFPWT | 863 |
| W917-03 | North | CDRH1 | AASGFTFSGSAMH | 873 |
| | | CDRH2 | RIRSKANSYATA | 874 |
| | | CDRH3 | TRLTDIFRLLGSNFYGMDV | 875 |
| | | CDRL1 | SGDALPKQYAY | 360 |
| | | CDRL2 | YKDSERPS | 361 |
| | | CDRL3 | QSADSSGTYVV | 876 |
| | Kabat | CDRH1 | GSAMH | 877 |
| | | CDRH2 | RIRSKANSYATAYAASVKG | 878 |
| | | CDRH3 | LTDIFRLLGSNFYGMDV | 879 |
| | | CDRL1 | SGDALPKQYAY | 360 |
| | | CDRL2 | KDSERPS | 366 |
| | | CDRL3 | QSADSSGTYVV | 876 |
| | IMGT | CDRH1 | GFTFSGSA | 880 |
| | | CDRH2 | IRSKANSYAT | 881 |
| | | CDRH3 | TRLTDIFRLLGSNFYGMDV | 875 |
| | | CDRL1 | ALPKQY | 369 |
| | | CDRL2 | KD | — |
| | | CDRL3 | QSADSSGTYVV | 876 |
| | Chothia | CDRH1 | GFTFSGS | 882 |
| | | CDRH2 | RSKANSYA | 883 |
| | | CDRH3 | LTDIFRLLGSNFYGMDV | 879 |
| | | CDRL1 | SGDALPKQYAY | 360 |
| | | CDRL2 | KDSERPS | 366 |
| | | CDRL3 | QSADSSGTYVV | 876 |
| | Contact | CDRH1 | SGSAMH | 884 |
| | | CDRH2 | WVGRIRSKANSYATA | 885 |
| | | CDRH3 | TRLTDIFRLLGSNFYGMD | 886 |
| | | CDRL1 | KQYAYWY | 74 |
| | | CDRL2 | LVIYKDSERP | 375 |
| | | CDRL3 | QSADSSGTYV | 887 |
| Z504-01 | North | CDRH1 | AASRFTFSRYAMT | 888 |
| | | CDRH2 | AISSTGDNPY | 889 |
| | | CDRH3 | AKDQGGADNNGYYYNFDY | 890 |
| | | CDRL1 | GGNDIGSKSVH | 891 |
| | | CDRL2 | YYDSDRPS | 741 |
| | | CDRL3 | QVWDSSSDHVV | 892 |
| | Kabat | CDRH1 | RYAMT | 893 |
| | | CDRH2 | AISSTGDNPYYADSVKG | 894 |
| | | CDRH3 | DQGGADNNGYYYNFDY | 895 |
| | | CDRL1 | GGNDIGSKSVH | 891 |
| | | CDRL2 | YDSDRPS | 743 |
| | | CDRL3 | QVWDSSSDHVV | 892 |
| | IMGT | CDRH1 | RFTFSRYA | 896 |
| | | CDRH2 | ISSTGDNP | 897 |
| | | CDRH3 | AKDQGGADNNGYYYNFDY | 890 |
| | | CDRL1 | DIGSKS | 898 |
| | | CDRL2 | YD | — |
| | | CDRL3 | QVWDSSSDHVV | 892 |
| | Chothia | CDRH1 | RFTFSRY | 899 |
| | | CDRH2 | SSTGDN | 900 |
| | | CDRH3 | DQGGADNNGYYYNFDY | 895 |
| | | CDRL1 | GGNDIGSKSVH | 891 |
| | | CDRL2 | YDSDRPS | 743 |
| | | CDRL3 | QVWDSSSDHVV | 892 |
| | Contact | CDRH1 | SRYAMT | 901 |
| | | CDRH2 | WVSAISSTGDNPY | 902 |
| | | CDRH3 | AKDQGGADNNGYYYNFD | 903 |
| | | CDRL1 | SKSVHWY | 745 |
| | | CDRL2 | QVIYYDSDRP | 904 |
| | | CDRL3 | QVWDSSSDHV | 905 |
| Z504-02 | North | CDRH1 | AASGFSFSTYAMN | 906 |
| | | CDRH2 | LFSGSGGDTY | 907 |
| | | CDRH3 | AKDLGVMFFGAFDI | 908 |
| | | CDRL1 | RASQSISSWLA | 861 |
| | | CDRL2 | YKASSLQS | 909 |
| | | CDRL3 | QQYNDYSVT | 910 |
| | Kabat | CDRH1 | TYAMN | 911 |
| | | CDRH2 | LFSGSGGDTYYADSVKG | 912 |
| | | CDRH3 | DLGVMFFGAFDI | 913 |
| | | CDRL1 | RASQSISSWLA | 861 |
| | | CDRL2 | KASSLQS | 914 |
| | | CDRL3 | QQYNDYSVT | 910 |
| | IMGT | CDRH1 | GFSFSTYA | 915 |
| | | CDRH2 | FSGSGGDT | 916 |
| | | CDRH3 | AKDLGVMFFGAFDI | 908 |
| | | CDRL1 | QSISSW | 866 |
| | | CDRL2 | KA | — |
| | | CDRL3 | QQYNDYSVT | 910 |
| | Chothia | CDRH1 | GFSFSTY | 917 |
| | | CDRH2 | SGSGGD | 918 |
| | | CDRH3 | DLGVMFFGAFDI | 913 |
| | | CDRL1 | RASQSISSWLA | 861 |
| | | CDRL2 | KASSLQS | 914 |
| | | CDRL3 | QQYNDYSVT | 910 |
| | Contact | CDRH1 | STYAMN | 919 |
| | | CDRH2 | WVSLFSGSGGDTY | 920 |
| | | CDRH3 | AKDLGVMFFGAFD | 921 |
| | | CDRL1 | SSWLAWY | 871 |
| | | CDRL2 | LLIYKASSLQ | 922 |
| | | CDRL3 | QQYNDYSV | 923 |
| Z538-01 | North | CDRH1 | AASGFTFTNYWMH | 924 |
| | | CDRH2 | RLNSDGSITG | 925 |
| | | CDRH3 | ARVGLRTLPAIENFDY | 926 |
| | | CDRL1 | RASQSISIYLN | 927 |
| | | CDRL2 | YAASSLQS | 642 |
| | | CDRL3 | QQSYRAVLT | 928 |
| | Kabat | CDRH1 | NYWMH | 929 |
| | | CDRH2 | RLNSDGSITGYADSVKG | 930 |
| | | CDRH3 | VGLRTLPAIENFDY | 931 |
| | | CDRL1 | RASQSISIYLN | 927 |
| | | CDRL2 | AASSLQS | 647 |
| | | CDRL3 | QQSYRAVLT | 928 |
| | IMGT | CDRH1 | GFTFTNYW | 932 |
| | | CDRH2 | LNSDGSIT | 933 |
| | | CDRH3 | ARVGLRTLPAIENFDY | 934 |
| | | CDRL1 | QSISIY | 935 |
| | | CDRL2 | AA | — |
| | | CDRL3 | QQSYRAVLT | 928 |
| | Chothia | CDRH1 | GFTFTNY | 936 |
| | | CDRH2 | NSDGSI | 937 |
| | | CDRH3 | VGLRTLPAIENFDY | 931 |
| | | CDRL1 | RASQSISIYLN | 927 |
| | | CDRL2 | AASSLQS | 647 |
| | | CDRL3 | QQSYRAVLT | 928 |
| | Contact | CDRH1 | TNYWMH | 938 |
| | | CDRH2 | WVSRLNSDGSITG | 939 |
| | | CDRH3 | ARVGLRTLPAIENFD | 940 |
| | | CDRL1 | SIYLNWY | 941 |
| | | CDRL2 | LLIYAASSLQ | 942 |
| | | CDRL3 | QQSYRAVL | 943 |
| Z538-02 | North | CDRH1 | AASGFIFSYYWMS | 944 |
| | | CDRH2 | NIKEDGSEKY | 945 |
| | | CDRH3 | ARAPLRNFDWPLDV | 946 |
| | | CDRL1 | TGTSSDVGANDYVS | 947 |
| | | CDRL2 | YDVSKRTS | 948 |
| | | CDRL3 | CSYGGRYV | 949 |
| | Kabat | CDRH1 | YYWMS | 950 |
| | | CDRH2 | NIKEDGSEKYYVDSVKG | 951 |
| | | CDRH3 | APLRNFDWPLDV | 952 |
| | | CDRL1 | TGTSSDVGANDYVS | 947 |
| | | CDRL2 | DVSKRTS | 953 |
| | | CDRL3 | CSYGGRYV | 949 |
| | IMGT | CDRH1 | GFIFSYYW | 954 |
| | | CDRH2 | IKEDGSEK | 955 |
| | | CDRH3 | ARAPLRNFDWPLDV | 946 |
| | | CDRL1 | SSDVGANDY | 956 |
| | | CDRL2 | DV | — |
| | | CDRL3 | CSYGGRYV | 949 |
| | Chothia | CDRH1 | GFIFSYY | 957 |
| | | CDRH2 | KEDGSE | 958 |
| | | CDRH3 | APLRNFDWPLDV | 952 |
| | | CDRL1 | TGTSSDVGANDYVS | 947 |
| | | CDRL2 | DVSKRTS | 953 |
| | | CDRL3 | CSYGGRYV | 949 |
| | Contact | CDRH1 | SYYWMS | 959 |
| | | CDRH2 | WVANIKEDGSEKY | 960 |
| | | CDRH3 | ARAPLRNFDWPLD | 961 |
| | | CDRL1 | VGANDYVSWY | 962 |
| | | CDRL2 | LIIYDVSKRT | 963 |
| | | CDRL3 | CSYGGRYV | 949 |
| Z573-01 | North | CDRH1 | KGSGYKFSSYWIA | 964 |
| | | CDRH2 | IIYPDDSDTT | 827 |
| | | CDRH3 | VRGGSAPH | 965 |
| | | CDRL1 | KSSQSVLYSSNNKNYLT | 966 |
| | | CDRL2 | YWASTRES | 967 |
| | | CDRL3 | QQHYTTPWT | 968 |
| | Kabat | CDRH1 | SYWIA | 969 |
| | | CDRH2 | IIYPDDSDTTYSPSFQG | 832 |
| | | CDRH3 | GGSAPH | 970 |
| | | CDRL1 | KSSQSVLYSSNNKNYLT | 966 |
| | | CDRL2 | WASTRES | 971 |
| | | CDRL3 | QQHYTTPWT | 968 |
| | IMGT | CDRH1 | GYKFSSYW | 972 |
| | | CDRH2 | IYPDDSDT | 324 |
| | | CDRH3 | VRGGSAPH | 965 |
| | | CDRL1 | QSVLYSSNNKNY | 973 |
| | | CDRL2 | WA | — |
| | | CDRL3 | QQHYTTPWT | 968 |
| | Chothia | CDRH1 | GYKFSSY | 974 |
| | | CDRH2 | YPDDSD | 327 |
| | | CDRH3 | GGSAPH | 970 |
| | | CDRL1 | KSSQSVLYSSNNKNYLT | 966 |
| | | CDRL2 | WASTRES | 971 |
| | | CDRL3 | QQHYTTPWT | 968 |
| | Contact | CDRH1 | SSYWIA | 975 |
| | | CDRH2 | WMGIIYPDDSDTT | 838 |
| | | CDRH3 | VRGGSAP | 976 |
| | | CDRL1 | LYSSNNKNYLTWY | 977 |
| | | CDRL2 | LLIYWASTRE | 978 |
| | | CDRL3 | QQHYTTPW | 979 |
| Z573-02 | North | CDRH1 | KASGYTFNTFYMH | 980 |
| | | CDRH2 | VINPSRGRTI | 981 |
| | | CDRH3 | ARDAGLGPYYYYMDV | 683 |
| | | CDRL1 | SGDGLTKQYAF | 68 |
| | | CDRL2 | YKDSERPS | 361 |
| | | CDRL3 | QSADRSDIYVV | 982 |
| | Kabat | CDRH1 | TFYMH | 983 |
| | | CDRH2 | VINPSRGRTIYAQKFQG | 984 |
| | | CDRH3 | DAGLGPYYYYMDV | 688 |
| | | CDRL1 | SGDGLTKQYAF | 684 |
| | | CDRL2 | KDSERPS | 366 |
| | | CDRL3 | QSADRSDIYVV | 982 |
| | IMGT | CDRH1 | GYTFNTFY | 985 |
| | | CDRH2 | INPSRGRT | 986 |
| | | CDRH3 | ARDAGLGPYYYYMDV | 683 |
| | | CDRL1 | GLTKQY | 691 |
| | | CDRL2 | KD | — |
| | | CDRL3 | QSADRSDIYVV | 982 |
| | Chothia | CDRH1 | GYTFNTF | 987 |
| | | CDRH2 | NPSRGR | 988 |
| | | CDRH3 | DAGLGPYYYYMDV | 688 |
| | | CDRL1 | SGDGLTKQYAF | 684 |
| | | CDRL2 | KDSERPS | 366 |
| | | CDRL3 | QSADRSDIYVV | 982 |
| | Contact | CDRH1 | NTFYMH | 989 |
| | | CDRH2 | WVGVINPSRGRTI | 990 |
| | | CDRH3 | ARDAGLGPYYYYMD | 696 |
| | | CDRL1 | KQYAFWY | 697 |
| | | CDRL2 | LVIYKDSERP | 375 |
| | | CDRL3 | QSADRSDIYVV | 982 |

TABLE 2
Antibody CDR encoding sequences

| | | | SEQ ID |
|---|---|---|---|
| Antibody | CDR | Encoding Sequence | NO |
| W056-03 | CDRH1 | GGAGTCACCTTCAGTCGTTTTGAC | 991 |
| | CDRH2 | ATTAGTAGTAGTGGTACTACCATA | 992 |
| | CDRH3 | GCGAGAGATGATTACACTGGCTGGTACTACTTTGACTC | 993 |
| | | C | |
| | CDRL1 | AGCTCCAACATCGGGGCAGGTTTTGAC | 994 |
| | CDRL2 | GGTAACAGT | — |
| | CDRL3 | CAGTCCTATGACAGCAGCCTGTATGTC | 996 |
| W056-04 | CDRH1 | GGGTTCTCACTCAGCACTAGTGGAATGTGT | 997 |
| | CDRH2 | ATTGATTGGGATGATGATAAA | 998 |
| | CDRH3 | GCACGCCAGGAACTAGTGGGAGCCTGGTTCGACCCC | 999 |
| | CDRL1 | AGCGGTGACGTTGGTGGTTATAATTAT | 1000 |
| | CDRL2 | GAGGTCAGT | — |
| | CDRL3 | ATCTCATATACAAGCAGCAGCATCTCTTGTGTC | 1002 |
| W056-05 | CDRH1 | GGATTCACCTTCAGTAGCTATGGC | 1003 |
| | CDRH2 | ATATCATATGATGGAAGTACTAAA | 1004 |
| | CDRH3 | GCGAAAGATGGCACAGCAGTGCTTGTGGACCGTCTTG | 1005 |
| | | ACTAC | |
| | CDRL1 | CAGGGCATTAGCAGTTAT | 1006 |
| | CDRL2 | GCTGCATCC | — |
| | CDRL3 | CAACAGCTTAATAGTTACCCGCTCACT | 1008 |
| W056-06 | CDRH1 | GGATACGACTTTGACACGTACTGG | 1009 |
| | CDRH2 | ATTTATCCTGATGACTCTGATACC | 1010 |
| | CDRH3 | GCGAGAGCAACTGGGGACTAT | 1111 |
| | CDRL1 | CAAAATGTTAGTACTTGG | 1112 |
| | CDRL2 | GAGGCGTCC | — |
| | CDRL3 | CAACAATATAGAAGTTATTCTCGAACC | 1114 |
| W056-08 | CDRH1 | GGGTTCTCACTCAGCACTAGTGGAATGTGT | 997 |
| | CDRH2 | ATTGATTGGGATGATGATAAA | 998 |
| | CDRH3 | GCACGCCAGGAACTAGTGGGAGCCTGGTTCGACCCC | 999 |
| | CDRL1 | AGCGGTGACGTTGGTGGTTATAATTAT | 1000 |
| | CDRL2 | GAGGTCAGT | — |
| | CDRL3 | ATCTCATATACAAGCAGCAGCATCTCTTGTGTC | 1002 |
| W056-09 | CDRH1 | GGATACGACTTTAACACCTACTGG | 1115 |
| | CDRH2 | ATCTACCCTGATGACTCTGATACC | 1116 |
| | CDRH3 | GCGAGAGCAACTGGGGACTAT | 1111 |
| | CDRL1 | CAAAATGTTAGTAGATGG | 1117 |
| | CDRL2 | GAGGCGTCC | |
| | CDRL3 | CAACAATATAGGAGTTATTCTCGAACT | 1118 |
| W056-10 | CDRH1 | GGAGCCACCTTCAGCAGCTATGCT | 1119 |
| | CDRH2 | GTCACCCCTATTATTGATACAACA | 1120 |
| | CDRH3 | GCGACGCGTGGGACGTATTACGATTTTTGGCCCAGTA | 1121 |
| | | TGGACGTC | |
| | CDRL1 | CAGGACATTAGGAATTAT | 1122 |
| | CDRL2 | GCTGCATCC | — |
| | CDRL3 | CAACAGTATAATAGTTACCCTCTCACT | 1123 |
| Z156-02 | CDRH1 | GGTGGGTCCTTCAGTGGTTACTAC | 1124 |
| | CDRH2 | ATCAATCATAGTGGAAGCACC | 1125 |
| | CDRH3 | GCGAGCATGAGTGGGAGCTACTACGGGGCCTTTGACT | 1126 |
| | | AC | |
| | CDRL1 | GCATTGCCAAAGCAATAT | 1127 |
| | CDRL2 | AAAGACAGT | |
| | CDRL3 | CAATCAGCAGACAGCAGTGGTACTTATTATGTC | 1129 |
| Z156-03 | CDRH1 | GGTGGGTCCTTCAGTGGTTACTAC | 1124 |
| | CDRH2 | ATCAATCATAGTGGAAGCACC | 1125 |
| | CDRH3 | GCGAGCATGAGTGGGAGCTACTACGGGGCCTTTGACT | 1126 |
| | | AC | |
| | CDRL1 | AGTGGCATCAATGTTGGTACCTACAGG | 1130 |
| | CDRL2 | TACAAATCAGACTCAGATAAG | 1131 |
| | CDRL3 | ATGATTTGGCACAGCAGCGCTTGGGTG | 1132 |
| Z249-01 | CDRH1 | GGATTTACCTTCAAGCACTATTCT | 1133 |
| | CDRH2 | ATATCTTATGATGGAAGCAAGACG | 1134 |
| | CDRH3 | GTGAGAGATCGGACGACTGGCCACGATTTCGGCTTTG | 1135 |
| | | ACTAC | |
| | CDRL1 | GCATTGTCGAAGAAATAT | 1136 |
| | CDRL2 | AAAGACAAT | — |
| | CDRL3 | CAATCAACAGACAGCGATACTTCTTATGTAATT | 1138 |
| Z249-02 | CDRH1 | GGAGGCTTCTTCAGCAGCTATCGT | 1139 |
| | CDRH2 | CTCGTCCCTATTTTCGAAACAGCC | 1140 |
| | CDRH3 | GCGAGAGGGTCGGGCAGTGGGCTTACATACTTCCAAA | 1141 |
| | | CC | |
| | CDRL1 | CAGAGTATTACCACCTACTAC | 1142 |
| | CDRL2 | GGTGCATCC | |
| | CDRL3 | CAGCAGTATGATAGTTCACCCACG | 1144 |
| Z249-03 | CDRH1 | GGTGGCTCCATCACCAGTATTAGTCACTAC | 1145 |
| | CDRH2 | ATGTATTACAGTGGGACCACC | 1146 |
| | CDRH3 | GCGAGACATGACGTTAGCAGGTTCGACCCC | 1147 |
| | CDRL1 | AGCAGTGACGTTGGTGGTTATAACTAT | 1148 |
| | CDRL2 | GATGTCACT | — |
| | CDRL3 | AGCTCATATACAACCAGCACTTGGGTG | 1150 |
| Z249-04 | CDRH1 | GGATTCACCTTCAGTAATTTTGAT | 1151 |
| | CDRH2 | ATTTCATATGATGAAAGTAAAAGA | 1152 |
| | CDRH3 | GCGACCGAGTCCACGCAATACTTTGGTGCTCTTGATAC | 1153 |
| | | C | |
| | CDRL1 | GGATTGGCAGACCAATAT | 1154 |
| | CDRL2 | AAGGACAGC | |
| | CDRL3 | CAATCAACAGACAGAAGTGGCTCTTATCTA | 1156 |
| Z249-05 | CDRH1 | GGATTCAACTTCAATTTTTACACT | 1157 |
| | CDRH2 | GTATTTTATGATGGGAGCAAAACC | 1158 |
| | CDRH3 | GTGAGAGATAGAACGACAGGACACGATTACGGCATGG | 1159 |
| | | ACGTC | |
| | CDRL1 | GGCTTGGCAAACCTATAT | 1160 |
| | CDRL2 | AAAGACACT | — |
| | CDRL3 | CAATCAGCAGACAGCAGTCGTTCTTCTGTGATC | 1162 |
| Z249-06 | CDRH1 | GGAGGCTTCTTCAGCAGCTATCGT | 1139 |
| | CDRH2 | CTCGTCCCTATTTTCGAAACAGCC | 1140 |
| | CDRH3 | GCGAGAGGGTCGGGCAGTGGGCTTACATACTTCCAAA | 1141 |
| | | CC | |
| | CDRL1 | CAGAGTATTACCACCTACTAC | 1142 |
| | CDRL2 | GGTGCATCC | — |
| | CDRL3 | CAGCAGTATGATAGTTCACCCACG | 1144 |
| Z308-03 | CDRH1 | GGGTTCTCACTCAGCACTGGTGGAGTGTGT | 1163 |
| | CDRH2 | ATTGATTGGGATAATGATAAA | 1164 |
| | CDRH3 | GCACGCATGACGGGCCTGTATAGTGACAACTGGAAGG | 1165 |
| | | GTTTTGATATC | |
| | CDRL1 | CAGAGTGTTGACAGCACTTAC | 1166 |
| | CDRL2 | AGTGCATCC | — |
| | CDRL3 | CAGCAGTATGGTAGCGCACCGGGGTACACT | 1168 |
| Z308-04 | CDRH1 | GGATACAGATTTACCAGTTACTGG | 1169 |
| | CDRH2 | ATCTATCCTCCTGACTCTGATACT | 1170 |
| | CDRH3 | GCGAGACAGGGAAGGGGATTTGACTAC | 1171 |
| | CDRL1 | CAAAACATTTACACTTAT | 1172 |
| | CDRL2 | ACTGCGTCC | — |
| | CDRL3 | CAGCAGAGTTACGATTCCCCTCGAACG | 1174 |
| Z308-05 | CDRH1 | GGGTTCTCACTCAACACTGCTGAAGTGTGT | 1175 |
| | CDRH2 | ATTGATTGGGATGATGATAAA | 998 |
| | CDRH3 | GCACGCATGACGGGCCTGTATACTGACAACTGGAAGG | 1176 |
| | | GTTTTGATATG | |
| | CDRL1 | CAGACTATTGACAGTACTTAC | 1177 |
| | CDRL2 | AGTGCATCC | |
| | CDRL3 | CAGCAGTATGGTCACGCACCGGGGTACACT | 1178 |
| Z504-03 | CDRH1 | GGAGGCACCTTCAGTAGATATGCT | 1179 |
| | CDRH2 | ATCATCCCTATGTTTGATATAACA | 1180 |
| | CDRH3 | GCGACTGAGAGTTGTAGTGGTGGTAACTGCTATTGGG | 1181 |
| | | ATCATGATGCTTTTGATATC | |
| | CDRL1 | CAGAGTCTTAGCAGCAGCTAC | 1182 |
| | CDRL2 | GGTGCATCC | — |
| | CDRL3 | CAGCAGTATGGCAGGTCACTTCCGAGGACG | 1183 |
| Z504-04 | CDRH1 | GGAGGCACCTTCAGCAGCTATGCT | 1184 |
| | CDRH2 | ATCATTCCTATCTTTGATATAGCA | 1185 |
| | CDRH3 | GCGAGTGAGAATTGTGGCGGTGGTAACTGTTACTGGG | 1186 |
| | | AGCATGATGCTTTTGACATC | |
| | CDRL1 | CAGAGTGTTAGCGGCGGCGAC | 1187 |
| | CDRL2 | GGTGCATCC | -- |
| | CDRL3 | CAGCACTATGGTAATTCACCTCCGAGGACG | 1188 |
| Z504-06 | CDRH1 | GGAGGCACCTTCAGCACCTATGCT | 1189 |
| | CDRH2 | ATCATTCCTATCTTTGATATAGCA | 1185 |
| | CDRH3 | GCGAGTGAGAATTGTAGCGGTGGTAACTGTTACTGGG | 1190 |
| | | AACATGATGCTTTTGACATC | |
| | CDRL1 | CAGAGTGTTAGCGGCGGCGAC | 1187 |
| | CDRL2 | GGTGCATCC | — |
| | CDRL3 | CAGCAGTATGGTAATTCACCTCCGAGGACG | 1191 |
| Z504-07 | CDRH1 | GGATACAGCTTTGCCAGTTTCTGG | 1192 |
| | CDRH2 | ATATATCCTAGTGACTCTGATACA | 1193 |
| | CDRH3 | GCGAAACATGGAGTCGGAGCAGCTATTGACCAC | 1194 |
| | CDRL1 | CAGGTCATTAGCGGCTAT | 1195 |
| | CDRL2 | ACTACATCC | — |
| | CDRL3 | CAACAGAGTTACAGTAGTCCTCGAACT | 1197 |
| Z513-01 | CDRH1 | GGTGGCTCCATTGGGGGTGGTCCTTACCAC | 1198 |
| | CDRH2 | GTGGAAACCAGTGGGGACACT | 1199 |
| | CDRH3 | GCGAGAGGAACAGCTATGGATGCTTTTGATATC | 1200 |
| | CDRL1 | CAGGATATTAGTAACTGG | 1201 |
| | CDRL2 | GTTGCATCC | |
| | CDRL3 | CAACAGGGTAACAGTTTCCCGCTCACT | 1203 |
| Z513-02 | CDRH1 | GGATTCAGCTTCAGTGGCTATGGC | 1204 |
| | CDRH2 | ATTTGGTATGATGGAAGTAGTAAA | 1205 |
| | CDRH3 | GCGAGAGACGTCCTCGGGGAGTTGCCCGAT | 1206 |
| | CDRL1 | CAGGGCATTAGCAATTCT | 1207 |
| | CDRL2 | GCTGCTTCC | — |
| | CDRL3 | CAACAGTATAATACCTACCCTCTCACT | 1209 |
| Z538-03 | CDRH1 | GGCTTTACCTTCAGTAATTATGGC | 1210 |
| | CDRH2 | ATATGGTTTGATGGACG --AACA | 1211 |
| | CDRH3 | GCGAGAGAAACATGGAGTTACGCCGGGGGGGTCTTTG | 1212 |
| | | ACTAC | |
| | CDRL1 | AGTGGGCACAGCGGTCACGCC | 1213 |
| | CDRL2 | GTTCACAGTGATGGCAGCCAC | 1214 |
| | CDRL3 | CAGACCTGGGACACTGGCCTTTGGGTG | 1215 |
| Z573-03 | CDRH1 | GGATACACCTTCACCACCTACTAT | 1216 |
| | CDRH2 | CTCAACCCTAGTCGTAATAGGACT | 1217 |
| | CDRH3 | GCGAGAGATGCGGGACTGGGTCCGTACTACTACTACA | 1218 |
| | | TGGACGTC | |
| | CDRL1 | GGATTGACAAAGCAATAT | 1219 |
| | CDRL2 | AAAGACAGT | |
| | CDRL3 | CAATCAGCAGACAGAACTGATATTTATGTGGTC | 1220 |
| Z582-03 | CDRH1 | GGAGGCACCTTCAGCAGCGCTACT | 1221 |
| | CDRH2 | ATCACCCCTATGTTTACTACAGCA | 1222 |
| | CDRH3 | GCGAGGCAATTTGGTCATGAAAGTCGTGATTCTTACTA | 1223 |
| | | CCTCTAC | |
| | CDRL1 | CAGACTCTTAGCAGCAGCTAC | 1224 |
| | CDRL2 | GGTGCATCC | — |
| | CDRL3 | CAGCAATATGGTAGTTCGCGGACG | 1225 |
| Z582-14 | CDRH1 | GGTTACTCCATCAGCAGTGGTTACTAC | 1226 |
| | CDRH2 | ATCTATCATAGTGGGAGCACC | 1227 |
| | CDRH3 | GCGAGATCTGCAAGAGCAGTGGTTATGGATGCTTTTCA | 1228 |
| | | TATC | |
| | CDRL1 | AGTGGCAGCATTGCCAGCAACTAT | 1229 |
| | CDRL2 | GAGGATAAC | |
| | CDRL3 | CAGTCTTATGATAATATCCATCCTTGGGTG | 1231 |
| Z582-15 | CDRH1 | GGTTACTCCATCAGCAGTGGTTACTAC | 1226 |
| | CDRH2 | ATCTATCATAGTGGGAGCACC | 1227 |
| | CDRH3 | GCGAGATCTGCAAGAGCAGTGGTTATGGATGCTTTTCA | 1228 |
| | | TATC | |
| | CDRL1 | AACATTGGGAGTAAAAGT | 1232 |
| | CDRL2 | TATGATAGC | — |
| | CDRL3 | CAGGTGTGGGATGGTAGTACTGATCATTTTTATGTC | 1234 |
| Z082-U1 | CDRH1 | GGTGGCTCCATCAGCAGTGGTGAATACTAC | 1235 |
| | CDRH2 | ATCTCTTACGCCGGGAATACC | 1236 |
| | CDRH3 | GCCAGAGATTTCGTTGGCACCAACTGGTACGACAGAT | 1237 |
| | | ATGCTTTTCATATC | |
| | CDRL1 | AGCAGTGATGTTGGTGGTTATCACTAT | 1238 |
| | CDRL2 | GATGTCACT | — |
| | CDRL3 | TGCTCATATGCAGGCAGCTACGCTTTTTGGGTT | 1239 |
| Z082-U5 | CDRH1 | GGATTCAATTTCATCGACTACGCT | 1240 |
| | CDRH2 | ATTACTAATGATGCGAAAAGCACA | 1241 |
| | CDRH3 | GCGAGAGGCCGCAGTCATTATTATGATAGTAGTGGATT | 1242 |
| | | TGACTAC | |
| | CDRL1 | AGCCTTGGCAGTAAAAGT | 1243 |
| | CDRL2 | GATGATAGT | — |
| | CDRL3 | CAGGTGTGGGATGCTGGTGCCCTTTGGGTG | 1245 |
| Z082-U6 | CDRH1 | GGCGACTCCATCAGCACTACTTCTTACTAC | 1246 |
| | CDRH2 | ATCAATTATAGTGGCAACACC | 1247 |
| | CDRH3 | GCGAGACGGGGGGTCCCAGCTGGTATGGTCCCGTCC | 1248 |
| | | TGGATCGACCCC | |
| | CDRL1 | GAGGACATTCGCAACTAT | 1249 |
| | CDRL2 | GATGCATCC | — |
| | CDRL3 | CAACAATATGATACTGTCCCTCTCACT | 1251 |
| W056-01 | CDRH1 | GGATACCACTTTCCCACCTACTGG | 1252 |
| | CDRH2 | ATCTATCCTCGTGACTCCGATACC | 1253 |
| | CDRH3 | GCGAGAGCAACTGGGGACTAC | 1254 |
| | CDRL1 | GAGAATGTTAGTACCTGG | 1255 |
| | CDRL2 | AAGGCGTCT | |
| | CDRL3 | CAACAATATAAGAGTTATTCTCGAACG | 1257 |
| W056-02 | CDRH1 | GGATACAGTTTCCCCACCTACTGG | 1258 |
| | CDRH2 | ATCTATCCAGATGACTCCGATACC | 1259 |
| | CDRH3 | GCGCGAGGTATGGGGGATCAC | 1260 |
| | CDRL1 | CAGAGCCTCCTCTTTGGTGATGGAGCGACCTAT | 1261 |
| | CDRL2 | AAAGTTTCC | — |
| | CDRL3 | ATGCAAGCTCTACATATGCCTCGGACG | 1263 |
| W917-01 | CDRH1 | GGATTCACCGTCAGTAGCAACTAC | 1264 |
| | CDRH2 | ATTTATAGCGGTGGTAGCACA | 1265 |
| | CDRH3 | GCGAGAGATCGGAAGATAGTAGTTAGGCGCGGGGGC | 1266 |
| | | TTAGATATC | |
| | CDRL1 | CAGGGTATTAGCAGTTAT | 1267 |
| | CDRL2 | GCTGCATCC | — |
| | CDRL3 | CAACAGTATTATAGTTACCCTCACACC | 1268 |
| W917-02 | CDRH1 | GGATTCACCTTCAGTAGCTATAGC | 1269 |
| | CDRH2 | ATTAGTAGTAGTAGTAGTACCATA | 1270 |
| | CDRH3 | GCGAGAGATGGGGCAGCAGCTGGGGTCAACCCC | 1271 |
| | CDRL1 | CAGAGTATTAGTAGCTGG | 1272 |
| | CDRL2 | AAGGCGTCT | |
| | CDRL3 | CAACAGTATAATAGTTTCCCGTGGACG | 1273 |
| W917-03 | CDRH1 | GGGTTCACCTTCAGTGGCTCTGCT | 1274 |
| | CDRH2 | ATTAGAAGCAAAGCTAACAGTTACGCGACA | 1275 |
| | CDRH3 | ACTAGACTAACCGATATTTTTCGACTGCTGGGCAGCAA | 1276 |
| | | CTTTTACGGTATGGACGTC | |
| | CDRL1 | GCATTGCCAAAGCAATAT | 1127 |
| | CDRL2 | AAAGACAGT | — |
| | CDRL3 | CAATCAGCAGACAGCAGTGGTACTTATGTGGTA | 1277 |
| Z504-01 | CDRH1 | CGATTCACCTTTAGCCGCTATGCC | 1278 |
| | CDRH2 | ATTAGCAGCACTGGTGACAACCCA | 1279 |
| | CDRH3 | GCGAAAGACCAGGGGGGGGCTGATAATAATGGTTATT | 1280 |
| | | ACTACAACTTTGACTAC | |
| | CDRL1 | GACATTGGAAGTAAAAGT | 1281 |
| | CDRL2 | TATGATAGC | |
| | CDRL3 | CAGGTGTGGGATAGTAGTAGTGATCATGTGGTA | 1282 |
| Z504-02 | CDRH1 | GGATTCAGCTTTAGCACCTATGCC | 1283 |
| | CDRH2 | TTTAGTGGGAGTGGTGGTGACACA | 1284 |
| | CDRH3 | GCGAAAGATCTAGGGGTTATGTTTTTCGGAGCTTTTGA | 1285 |
| | | TATC | |
| | CDRL1 | CAGAGTATTAGTAGCTGG | 1272 |
| | CDRL2 | AAGGCGTCT | — |
| | CDRL3 | CAACAGTATAATGATTATTCGGTCACT | 1286 |
| Z538-01 | CDRH1 | GGATTCACCTTCACTAACTACTGG | 1287 |
| | CDRH2 | CTTAATTCTGATGGGAGTATCACA | 1288 |
| | CDRH3 | GCAAGAGTAGGTCTACGGACTTTGCCCGCAATCGAAA | 1289 |
| | | ACTTTGACTAC | |
| | CDRL1 | CAGAGCATTAGCATCTAT | 1290 |
| | CDRL2 | GCTGCATCC | — |
| | CDRL3 | CAACAGAGTTACAGGGCCGTGCTCACT | 1291 |
| Z538-02 | CDRH1 | GGATTCATCTTTAGTTACTATTGG | 1292 |
| | CDRH2 | ATAAAGGAAGACGGAAGTGAGAAG | 1293 |
| | CDRH3 | GCGAGGGCGCCCTTACGGAATTTTGACTGGCCACTCG | 1294 |
| | | ACGTC | |
| | CDRL1 | AGCAGTGATGTTGGTGCTAATGACTAT | 1295 |
| | CDRL2 | GATGTCTCT | |
| | CDRL3 | TGCTCGTATGGGGGCAGGTATGTC | 1297 |
| Z573-01 | CDRH1 | GGATACAAATTTAGCAGTTATTGG | 1298 |
| | CDRH2 | ATCTATCCTGATGACTCTGATACC | 1299 |
| | CDRH3 | GTGAGAGGGGGGAGTGCCCCCCAT | 1300 |
| | CDRL1 | CAGAGTGTTTTATACAGCTCCAACAATAAGAACTAC | 1301 |
| | CDRL2 | TGGGCTTCT | — |
| | CDRL3 | CAACAACATTATACTACTCCGTGGACG | 1303 |
| Z573-02 | CDRH1 | GGATACACCTTCAACACCTTTTAT | 1304 |
| | CDRH2 | ATCAACCCTAGTCGTGGTAGGACA | 1305 |
| | CDRH3 | GCGAGAGATGCGGGACTTGGTCCGTACTACTACTACA | 1306 |
| | | TGGACGTC | |
| | CDRL1 | GGATTGACAAAGCAATAT | 1219 |
| | CDRL2 | AAAGACAGT | |
| | CDRL3 | CAATCAGCAGACAGAAGTGATATTTATGTGGTA | 1307 |

CDR predictions were generated using the program SAbPrep http://opig.stats.ox.ac.uk/webapps/newsabdab/sabpred/). ABodyBuilder within SAbPred was used.

The carboxy-terminal portion of each chain of a naturally occurring antibody defines a constant region, which can be responsible for effector function particularly in the heavy chain (the Fc). Examples of effector functions include: C1q binding and complement dependent cytotoxicity (CDC); antibody-dependent cell-mediated cytotoxicity (ADCC); phagocytosis; down regulation of cell surface receptors (e.g., B-cell receptors); and B-cell activation.

Human light chains are classified as kappa (Igκ) and lambda (Igλ) light chains. In particular embodiments, a human Igκ Fc region includes the sequence:

(SEQ ID NO: 171)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.

In particular embodiments, a human Igλ Fc region includes the sequence:

(SEQ ID NO: 172)
SVSVSPGQTARITCSGDALPKKYAYWYQQKSGQAPVLVIYEDNRRPSGI
PERFSGSSSRTLATLTISGAQVEDEADYYCYSTDSSGNHVVFGGGTKLT
VLRQPKAAPSVTSVPT.

Heavy chains are classified as mu, delta, gamma, alpha, or epsilon, and define the antibody's isotype as IgM, IgD, IgG, IgA, and IgE, respectively. IgG has several subclasses, including, IgG1, IgG2, IgG3, and IgG4. IgM has subclasses including IgM1 and IgM2. IgA is similarly subdivided into subclasses including IgA1 and IgA2.

In particular embodiments, a human IgG1 Fc region includes the sequence:

(SEQ ID NO: 173)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

In particular embodiments, a human IgG1 Fc region includes the sequence:

(SEQ ID NO: 174)
THTCPPCPAPEFFGGPSVFFFPPKPKDTFMISRTPEVTCVVVDVSHEDP
EVKFNWYVDGVEVHNAKTKPREEQYNSTYRVVSVETVFHQDWENGKEYK
CKVSNKAFPVPIEKTISKAKGQPREPQVYTLPPSREEMTKNQVSLTCLV
KGFYPSDIAVEWESNGQPENNYKTTPPVLDSDGPFFLYSKLTVDKSRWQ
QGNVFSCSVMHEALHNHYTQKSLSLSPGK.

In particular embodiments, a human IgG2 Fc region includes the amino acid sequence:

(SEQ ID NO: 175)
PAPPVAGPSVFLFPPKPKDTLMISRTPEVTCVVVDVSHEDPEVQFNWYV
DGVEVHNAKTKPREEQFNSTFRVVSVLTVVHQDWLNGKEYKCKVSNKGL
PAPIEKTISKTKGQPREPQVYTLPPSREEMTKNQVSLTCLVKGFYPSDI
AVEWESNGQPENNYKTTPPMLDSDGSFFLYSKLTVDKSRWQQGNVFSCS
VMHEALHNHYTQKSLSLSPGK

In particular embodiments, a human IgG3 Fc region includes the amino acid sequence:

(SEQ ID NO: 176)
PAPELLGGPSVFLFPPKPKDTLMISRTPEVTCVVVDVSHEDPEVQFKWY
VDGVEVHNAKTKPREEQFNSTFRVVSVLTVLHQDWLNGKEYKCKVSNKA
LPAPIEKTISKTKGQPREPQVYTLPPSREEMTKNQVSLTCLVKGFYPSD
IAVEWESSGQPENNYNTTPPMLDSDGSFFLYSKLTVDKSRWQQGNIFSC
SVMHEALHNRFTQKSLSLSPGK.

In particular embodiments, a human IgG4 Fc region includes the amino acid sequence:

(SEQ ID NO: 177)
PAPEFLGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKG
LPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSC
SVMHEALHNHYTQKSLSLSLGK.

Within full-length light and heavy chains, the variable and constant regions are joined by a “J” region of amino acids, with the heavy chain also including a “D” region of amino acids. See, e.g., Fundamental Immunology, Ch. 7 (Paul, W., ed., 2nd ed. Raven Press, N.Y. (1989)).

Unless otherwise indicated, the term “antibody” includes (in addition to antibodies having two full-length heavy chains and two full-length light chains as described above) variants, derivatives, and fragments thereof, examples of which are described below. Furthermore, unless explicitly excluded, antibodies can include monoclonal antibodies, human antibodies, bispecific antibodies, trispecific antibodies, tetraspecific antibodies, multi-specific antibodies, polyclonal antibodies, linear antibodies, minibodies, domain antibodies, synthetic antibodies, chimeric antibodies, antibody fusions, and fragments thereof, respectively. In particular embodiments, antibodies can include oligomers or multiplexed versions of antibodies.

A monoclonal antibody refers to an antibody obtained from a population of substantially homogeneous antibodies, i.e., the individual antibodies including the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which include different antibodies directed against different epitopes, each monoclonal antibody of a monoclonal antibody preparation is directed against a single epitope on an antigen. Thus, the modifier “monoclonal” indicates the character of the antibody as being obtained from a substantially homogeneous population of antibodies and is not to be construed as requiring production of the antibody by any particular method. For example, monoclonal antibodies can be made by a variety of techniques, including the hybridoma method, recombinant DNA methods, phage-display methods, and methods utilizing transgenic animals containing all or part of the human immunoglobulin loci.

A “human antibody” is one which includes an amino acid sequence which corresponds to that of an antibody produced by a human or a human cell or derived from a non-human source that utilizes human antibody repertoires or other human antibody-encoding sequences.

A “human consensus framework” is a framework that represents the most commonly occurring amino acid residues in a selection of human immunoglobulin VL or VH framework sequences. Generally, the selection of human immunoglobulin V_{L} or V_{H} sequences is from a subgroup of variable domain sequences. The subgroup of sequences can be a subgroup as in Kabat et al., Sequences of Proteins of Immunological Interest, Fifth Edition, NIH Publication 91-3242, Bethesda Md. (1991), vols. 1-3. In particular embodiments, for the V_{L}, the subgroup is subgroup kappa I as in Kabat et al., supra. In particular embodiments, for the V_{H}, the subgroup is subgroup Ill as in Kabat et al., supra.

Antibodies disclosed herein can be utilized to prepare various forms of relevant binding domain molecules. For example, particular embodiments can include binding fragments of an antibody, e.g., Fv, Fab, Fab′, F(ab′)₂, and single chain Fv fragments (scFvs) or any biologically effective fragments of an immunoglobulin that bind specifically to an epitope described herein.

In particular embodiments, an antibody fragment is used. An “antibody fragment” denotes a portion of a full-length antibody that retains the ability to bind to an epitope. Antibody fragments can be made by various techniques, including proteolytic digestion of an intact antibody as well as production by recombinant host-cells (e.g., mammalian suspension cell lines, E. coli or phage), as described herein. Antibody fragments can be screened for their binding properties in the same manner as intact antibodies. Examples of antibody fragments include Fv, scFv, Fab, Fab′, Fab′-SH, F(ab′)₂; diabodies; and linear antibodies.

A single chain variable fragment (scFv) is a fusion protein of the variable regions of the heavy and light chains of immunoglobulins connected with a short linker peptide. Fv fragments include the V_{L} and V_{H} domains of a single arm of an antibody but lack the constant regions. Although the two domains of the Fv fragment, V_{L} and V_{H}, are coded by separate genes, they can be joined, using, for example, recombinant methods, by a synthetic linker that enables them to be made as a single protein chain in which the V_{L} and V_{H} regions pair to form monovalent molecules (single chain Fv (scFv)). For additional information regarding Fv and scFv, see e.g., Bird, et al., Science 242:423-426, 1988; Huston, et al., Proc. Natl. Acad. Sci. USA 85:5879-5883, 1988; Plueckthun, in The Pharmacology of Monoclonal Antibodies, vol. 113, Rosenburg and Moore (eds), Springer-Verlag, New York), (1994) 269-315; WO 1993/16185; U.S. Pat. Nos. 5,571,894; and 5,587,458.

Linker sequences that are used to connect the VL and VH of an scFv are generally five to 35 amino acids in length. In particular embodiments, a VL-VH linker includes from five to 35, ten to 30 amino acids or from 15 to 25 amino acids. Variation in the linker length may retain or enhance activity, giving rise to superior efficacy in activity studies. Linker sequences of scFv are commonly Gly-Ser linkers, described in more detail elsewhere herein.

Additional examples of antibody-based binding domain formats include scFv-based grababodies and soluble VH domain antibodies. These antibodies form binding regions using only heavy chain variable regions. See, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

A Fab fragment is a monovalent antibody fragment including V_{L}, V_{H}, CL and CH1 domains. A F(ab′)₂ fragment is a bivalent fragment including two Fab fragments linked by a disulfide bridge at the hinge region. For discussion of Fab and F(ab′)₂ fragments having increased in vivo half-life, see U.S. Pat. No. 5,869,046. Diabodies include two epitope-binding sites that may be bivalent. See, for example, EP 0404097; WO1993/01161; and Holliger, et al., Proc. Natl. Acad. Sci. USA 90:6444-6448, 1993. Dual affinity retargeting antibodies (DART™; based on the diabody format but featuring a C-terminal disulfide bridge for additional stabilization (Moore et al., Blood 117:4542-51, 2011)) can also be used. Antibody fragments can also include isolated CDRs. For a review of antibody fragments, see Hudson, et al., Nat. Med. 9:129-134, 2003.

In particular embodiments, one or more amino acid modifications may be introduced into the Fc region of an antibody, thereby generating an Fc region variant. The Fc region variant may include a human Fc region sequence (e.g., a human IgG1, IgG2, IgG3 or IgG4 Fc region) including an amino acid modification (e.g., a substitution) at one or more amino acid positions. Numerous Fc modifications are known in the art, and a representative sampling of such possible modifications are described herein.

In particular embodiments, variants (including Fc variants) have been modified from a reference sequence to produce an administration benefit. Exemplary administration benefits can include (1) reduced susceptibility to proteolysis, (2) reduced susceptibility to oxidation, (3) altered binding affinity for forming protein complexes, (4) altered binding affinities, (5) reduced immunogenicity; and/or (6) extended half-life. While the disclosure below describes these modifications in terms of their application to antibodies, when applicable to another particular T antigen of the Merkel cell polyomavirus binding domain format (e.g., bispecific antibodies), the modifications can also be applied to these other formats.

In particular embodiments the antibodies can be mutated to increase their affinity for Fc receptors. Exemplary mutations that increase the affinity for Fc receptors include: G236A/S239D/A330L/1332E (GASDALIE). Smith et al., Proceedings of the National Academy of Sciences of the United States of America, 109(16), 6181-6186, 2012. In particular embodiments, an antibody variant includes an Fc region with one or more amino acid substitutions which improve ADCC, e.g., substitutions at positions 298, 333, and/or 334 of the Fc region (EU numbering of residues). In particular embodiments, alterations are made in the Fc region that result in altered C1q binding and/or Complement Dependent Cytotoxicity (CDC), e.g., as described in U.S. Pat. No. 6,194,551, WO 99/51642, and Idusogie et al., J. Immunol. 164: 4178-4184, 2000.

In particular embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further below. In particular embodiments, residue 5400 (EU numbering) of the heavy chain Fc region is selected. Cysteine engineered antibodies may be generated as described, e.g., in U.S. Pat. No. 7,521,541.

Antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at position 297 in the Fc region (Eu numbering of Fc region residues); however, Asn297 may also be located ±3 amino acids upstream or downstream of position 297, i.e., between positions 294 and 300, due to minor sequence variations in antibodies. Such fucosylation variants may have improved ADCC function. See, e.g., WO2000/61739; WO 2001/29246; WO2002/031140; US2002/0164328; WO2003/085119; WO2003/084570; US2003/0115614; US2003/0157108; US2004/0093621; US2004/0110704; US2004/0132140; US2004/0110282; US2004/0109865; WO2005/035586; WO2005/035778; WO2005/053742; Okazaki et al., J. Mol. Biol. 336:1239-1249 (2004); and Yamane-Ohnuki et al., Biotech. Bioeng. 87: 614 (2004). Examples of cell lines capable of producing defucosylated antibodies include Lec13 CHO cells deficient in protein fucosylation (Ripka et al., Arch. Biochem. Biophys. 249:533-545, 1986, and knockout cell lines, such as alpha-1,6-fucosyltransferase gene, FUT8, knockout CHO cells (see, e.g., Yamane-Ohnuki et al., Biotech. Bioeng. 87: 614, 2004; Kanda et al., Biotechnol. Bioeng., 94(4):680-688, 2006; and WO2003/085107).

In particular embodiments, modified antibodies include those wherein one or more amino acids have been replaced with a non-amino acid component, or where the amino acid has been conjugated to a functional group or a functional group has been otherwise associated with an amino acid. The modified amino acid may be, e.g., a glycosylated amino acid, a PEGylated amino acid, a farnesylated amino acid, an acetylated amino acid, a biotinylated amino acid, an amino acid conjugated to a lipid moiety, or an amino acid conjugated to an organic derivatizing agent. Amino acid(s) can be modified, for example, co-translationally or post-translationally during recombinant production (e.g., N-linked glycosylation at N—X-S/T motifs during expression in mammalian cells) or modified by synthetic means. The modified amino acid can be within the sequence or at the terminal end of a sequence. Modifications also include nitrited constructs.

In particular embodiments, variants include glycosylation variants wherein the number and/or type of glycosylation site has been altered compared to the amino acid sequences of a reference sequence. In particular embodiments, glycosylation variants include a greater or a lesser number of N-linked glycosylation sites than the reference sequence. An N-linked glycosylation site is characterized by the sequence: Asn-X-Ser or Asn-X-Thr, wherein the amino acid residue designated as X can be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (e.g., those that are naturally occurring) are eliminated and one or more new N-linked sites are created. Additional antibody variants include cysteine variants wherein one or more cysteine residues are deleted from or substituted for another amino acid (e.g., serine) as compared to the reference sequence. These cysteine variants can be useful when antibodies must be refolded into a biologically active conformation such as after the isolation of insoluble inclusion bodies. These cysteine variants generally have fewer cysteine residues than the reference sequence, and typically have an even number to minimize interactions resulting from unpaired cysteines.

PEGylation particularly is a process by which polyethylene glycol (PEG) polymer chains are covalently conjugated to other molecules such as proteins. Several methods of PEGylating proteins have been reported in the literature. For example, N-hydroxy succinimide (NHS)-PEG was used to PEGylate the free amine groups of lysine residues and N-terminus of proteins; PEGs bearing aldehyde groups have been used to PEGylate the amino-termini of proteins in the presence of a reducing reagent; PEGs with maleimide functional groups have been used for selectively PEGylating the free thiol groups of cysteine residues in proteins; and site-specific PEGylation of acetyl-phenylalanine residues can be performed.

Covalent attachment of proteins to PEG has proven to be a useful method to increase the half-lives of proteins in the body (Abuchowski, et al., Cancer Biochem. Biophys., 1984, 7:175-186; Hershfield, et al., N. Engl. J. Medicine, 1987, 316:589-596; and Meyers, et al., Clin. Pharmacol. Ther., 49:307-313, 1991). The attachment of PEG to proteins not only protects the molecules against enzymatic degradation, but also reduces their clearance rate from the body. The size of PEG attached to a protein has significant impact on the half-life of the protein. The ability of PEGylation to decrease clearance is generally not a function of how many PEG groups are attached to the protein, but the overall molecular weight of the altered protein. Usually the larger the PEG is, the longer the in vivo half-life of the attached protein. In addition, PEGylation can also decrease protein aggregation (Suzuki et al., Biochem. Bioph. Acta 788:248, 1984), alter protein immunogenicity (Abuchowski et al., J. Biol. Chem. 252: 3582, 1977), and increase protein solubility as described, for example, in PCT Publication No. WO 92/16221).

Several sizes of PEGs are commercially available (Nektar Advanced PEGylation Catalog 2005-2006; and NOF DDS Catalogue Ver 7.1), which are suitable for producing proteins with targeted circulating half-lives. A variety of active PEGs have been used including mPEG succinimidyl succinate, mPEG succinimidyl carbonate, and PEG aldehydes, such as mPEG-propionaldehyde.

In particular embodiments, the antibody can be fused or coupled to an Fc polypeptide that includes amino acid alterations that extend the in vivo half-life of an antibody that contains the altered Fc polypeptide as compared to the half-life of a similar antibody containing the same Fc polypeptide without the amino acid alterations. In particular embodiments, Fc polypeptide amino acid alterations can include M252Y, S254T, T256E, M428L, and/or N434S and can be used together, separately or in any combination. For example, M428L/N434S is a pair of mutations that increase the half-life of antibodies in serum, as described in Zalevsky et al., Nature Biotechnology 28, 157-159, 2010. Other alterations that can be helpful are described in U.S. Pat. Nos. 7,083,784, 7,670,600, US Publication No. 2010/0234575, PCT/US2012/070146, and Zwolak, Scientific Reports 7: 15521, 2017. In particular embodiments, any substitution at one of the following amino acid positions in an Fc polypeptide can be considered an Fc alteration that extends half-life: 250, 251, 252, 259, 307, 308, 332, 378, 380, 428, 430, 434, 436. Each of these alterations or combinations of these alterations can be used to extend the half-life of a bispecific antibody as described herein.

(ii) B Cells. In particular embodiments, B cells are genetically modified to express a binding domain that binds the T antigen of Merkel cell polyomavirus.

After leaving the bone marrow, a B cell acts as an antigen presenting cell (APC) and internalizes antigens. Antigen is taken up by the B cell through receptor-mediated endocytosis and processed. Antigen is processed into antigenic peptides, loaded onto MHC II molecules, and presented on the B cell extracellular surface to CD4⁺ T helper cells. These T cells bind to the MHC II/antigen molecule and cause activation of the B cell. Upon stimulation by a T cell, the activated B cell begins to differentiate into more specialized cells. Germinal center B cells may differentiate into long-lived memory B cells or plasma cells. Further, secondary immune stimulation may result in the memory B cells giving rise to additional plasma cells. The formation of plasma cells from either memory or non-memory B cells is preceded by the formation of precursor plasmablasts that eventually differentiate into plasma cells, which produce large volumes of antibodies (see e.g., Trends Immunol. 2009 June; 30(6): 277-285; Nature Reviews, 2005, 5:231-242). Plasmablasts secrete more antibodies than B cells, but less than plasma cells. They divide rapidly, and they continue to internalize antigens and present antigens to T cells. Plasmablasts have the capacity to migrate to sites of chemokine production (e.g., in bone marrow) whereby they may differentiate into long-lived plasma cells. Ultimately, a plasmablast may either remain as a plasmablast for several days and then die or irrevocably differentiate into a mature, fully differentiated plasma cell. Specifically, plasmablasts that are able home to tissues containing plasma cell survival niches (e.g., in bone marrow) are able to displace resident plasma cells in order to become long lived plasma cells, which may continue to secrete high levels of proteins for years.

CD19 is an example of a protein expressed by B cells but few other cells of the body. Thus, CD19 can be used to identify, isolate, and/or target B cells. B220 is a useful marker to identify, isolate, and/or target mouse B cells. Binding domains that bind CD19 can be derived from antibodies known in the art. For example, blinatumomab, tafasitamab and loncastuximab tesirine are monoclonal antibodies that target CD19.

Human naïve B cells can be identified, isolated, and/or targeted by the marker profile IgM+IgD+CD27−. Mouse naïve B cells can be identified, isolated, and/or targeted by the marker profile CD38+GL7− IgM+IgD+. Human B1 B cells can be identified, isolated, and/or targeted by the marker profile CD5+CD43+. Mouse B1 B cells can be identified, isolated, and/or targeted by the marker profile CD43+B220^{lo}. Human marginal zone B cells can be identified, isolated, and/or targeted by the marker profile CD21+++IgM++IgD-CD27+. Mouse marginal zone B cells can be identified, isolated, and/or targeted by the marker profile CD21+++IgM++IgD-. Particular embodiments may utilize the CD19⁺CD27⁺CD21^{lo} marker profile to identify, isolate, and/or target B cells. In particular embodiments, CD45 is a marker used for identifying, isolating, and/or targeting cell types. Different mouse strains express different versions of the protein called CD45, termed CD45.1 and CD45.2. In particular embodiments, B cells from a mouse that expresses CD45.2 will be taken and transferred into a mouse that expresses CD45.1. By marking CD45.1 and CD45.2 with different fluorescent molecules, one can identify the cells that came from the donor animal because they express CD45.2 but not CD45.1.

Terminally differentiated plasma cells typically do not express common pan-B cell markers, such as CD19 and CD20, and express relatively few surface antigens. Plasma cells express CD38, CD78, CD138 and interleukin-6 receptor (IL-6R) and lack expression of CD45, and these markers can be used, e.g., by flow cytometry, to identify plasma cells. CD27 is also a good marker for plasma cells as naïve B cells are CD27−, memory B cells are CD27+ and plasma cells are CD27++.

Memory B cell subsets may also express surface IgG, IgM and IgD, whereas plasma cells do not express these markers on the cell surface. CD38 and CD138 are expressed at high levels on plasma cells (See Wikipedia, The Free Encyclopedia., “Plasma cell” Page Version ID: 404969441; Date of last revision: 30 Dec. 2010 09:54 UTC, retrieved Jan. 4, 2011; See also: Jourdan et al., Blood. 2009 Dec. 10; 114(25):5173-81; Trends Immunol. 2009 June; 30(6): 277-285; Nature Reviews, 2005, 5:231-242; Nature Med. 2010, 16:123-129; Neuberger, M. S; Honjo, T; Alt, Frederick W. (2004). Molecular biology of B cells. Amsterdam: Elsevier, pp. 189-191; Bertil Glader; Greer, John G; John Foerster; Rodgers, George G; Paraskevas, Frixos (2008). Wintrobe's Clinical Hematology, 2-Vol. Set. Hagerstwon, Md.: Lippincott Williams & Wilkins. pp. 347; Walport, Mark; Murphy, Kenneth; Janeway, Charles; Travers, Paul J. (2008). Janeway's immunobiology. New York: Garland Science, pp. 387-388; Rawstron AC (May 2006). “Immunophenotyping of plasma cells”. Curr Protoc Cytom).

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

In particular embodiments, B cells can be patient-derived cells (autologous) or allogeneic. In particular embodiments, B cells can be genetically modified in vivo or ex vivo.

(iii) Other Immune Cells. In addition to B cells, other immune cells include T-cells, natural killer (NK) cells, NK-T cells, monocytes/macrophages, lymphocytes, hematopoietic stem cells (HSCs), hematopoietic progenitor cells (HPC), and/or a mixture of HSC and HPC (i.e., HSPC). In particular embodiments immune cells are genetically modified to express a chimeric antigen receptor (CAR). In particular embodiments, T cells are genetically modified to express an engineered T cell receptor (eTCR). In particular embodiments, the CAR or eTCR include a binding domain that binds the T antigen of Merkel cell polyomavirus.

Several different subsets of T-cells have been discovered, each with a distinct function. For example, a majority of T-cells have a T-cell receptor (TCR) existing as a complex of several proteins. The actual T-cell receptor is composed of two separate peptide chains, which are produced from the independent T-cell receptor alpha and beta (TCRα and TCRβ) genes and are called α- and β-TCR chains.

γδ T-cells represent a small subset of T-cells that possess a distinct T-cell receptor (TCR) on their surface. In γδ T-cells, the TCR is made up of one γ-chain and one δ-chain. This group of T-cells is much less common (2% of total T-cells) than the αβ T-cells.

CD3 is expressed on all mature T cells. Activated T-cells express 4-1BB (CD137), CD69, and CD25. CD5 and transferrin receptor are also expressed on T-cells.

T-cells can further be classified into helper cells (CD4+ T-cells) and cytotoxic T-cells (CTLs, CD8+ T-cells), which include cytolytic T-cells. T helper cells assist other white blood cells in immunologic processes, including maturation of B cells into plasma cells and activation of cytotoxic T-cells and macrophages, among other functions. These cells are also known as CD4+ T-cells because they express the CD4 protein on their surface. Helper T-cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T-cells destroy virally infected cells and tumor cells and are also implicated in transplant rejection. These cells are also known as CD8+ T-cells because they express the CD8 glycoprotein on their surface. These cells recognize their targets by binding to antigen associated with MHC class I, which is present on the surface of nearly every cell of the body.

“Central memory” T-cells (or “TCM”) refers to an antigen experienced CTL that expresses CD62L or CCR7 and CD45RO on the surface thereof and does not express or has decreased expression of CD45RA as compared to naïve cells. In particular embodiments, central memory cells are positive for expression of CD62L, CCR7, CD25, CD127, CD45RO, and CD95, and have decreased expression of CD45RA as compared to naïve cells.

“Effector memory” T-cell (or “TEM”) refers to an antigen experienced T-cell that does not express or has decreased expression of CD62L on the surface thereof as compared to central memory cells and does not express or has decreased expression of CD45RA as compared to a naïve cell. In particular embodiments, effector memory cells are negative for expression of CD62L and CCR7, compared to naïve cells or central memory cells, and have variable expression of CD28 and CD45RA. Effector T-cells are positive for granzyme B and perforin as compared to memory or naïve T-cells.

“Naïve” T-cells refers to a non-antigen experienced T cell that expresses CD62L and CD45RA and does not express CD45RO as compared to central or effector memory cells. In particular embodiments, naïve CD8+T lymphocytes are characterized by the expression of phenotypic markers of naïve T-cells including CD62L, CCR7, CD28, CD127, and CD45RA.

Natural killer cells (also known as NK cells, K cells, and killer cells) are activated in response to interferons or macrophage-derived cytokines. They serve to contain viral infections while the adaptive immune response is generating antigen-specific cytotoxic T cells that can clear the infection. NK cells express CD8, CD16 and CD56 but do not express CD3.

NK cells include NK-T cells. NK-T cells are a specialized population of T cells that express a semi invariant T cell receptor (TCR ab) and surface antigens typically associated with natural killer cells. NK-T cells contribute to antibacterial and antiviral immune responses and promote tumor-related immunosurveillance or immunosuppression. Like natural killer cells, NK-T cells can also induce perforin-, Fas-, and TNF-related cytotoxicity. Activated NK-T cells are capable of producing IFN-γ and IL-4. In particular embodiments, NK-T cells are CD3+/CD56+.

Macrophages (and their precursors, monocytes) reside in every tissue of the body (in certain instances as microglia, Kupffer cells and osteoclasts) where they engulf apoptotic cells, pathogens and other non-self-components. Monocytes/macrophages express CD11b, F4/80; CD68; CD11c; IL-4Ra; and/or CD163.

Immature dendritic cells (i.e., pre-activation) engulf antigens and other non-self-components in the periphery and subsequently, in activated form, migrate to T-cell areas of lymphoid tissues where they provide antigen presentation to T cells. Dendritic cells express CD1a, CD1b, CD1c, CD1d, CD21, CD35, CD39, CD40, CD86, CD101, CD148, CD209, and DEC-205.

Hematopoietic Stem/Progenitor Cells or HSPC refer to a combination of hematopoietic stem cells and hematopoietic progenitor cells.

Hematopoietic stem cells refer to undifferentiated hematopoietic cells that are capable of self-renewal either in vivo, essentially unlimited propagation in vitro, and capable of differentiation to all other hematopoietic cell types.

A hematopoietic progenitor cell is a cell derived from hematopoietic stem cells or fetal tissue that is capable of further differentiation into mature cell types. In certain embodiments, hematopoietic progenitor cells are CD24^{lo} Lin⁻ CD117⁺ hematopoietic progenitor cells. HPC can differentiate into (i) myeloid progenitor cells which ultimately give rise to monocytes and macrophages, neutrophils, basophils, eosinophils, erythrocytes, megakaryocytes/platelets, or dendritic cells; or (ii) lymphoid progenitor cells which ultimately give rise to T-cells, B-cells, and NK-cells.

HSPC can be positive for a specific marker expressed in increased levels on HSPC relative to other types of hematopoietic cells. For example, such markers include CD34, CD43, CD45RO, CD45RA, CD59, CD90, CD109, CD117, CD133, CD166, HLA DR, or a combination thereof. Also, the HSPC can be negative for an expressed marker relative to other types of hematopoietic cells. For example, such markers include Lin, CD38, or a combination thereof.

In particular embodiments, immune cells can be patient-derived cells (autologous) or allogeneic. In particular embodiments, immune cells can be genetically modified in vivo or ex vivo.

(iv) Recombinant Receptors. In particular embodiments, a recombinant receptor includes a CAR or an eTCR. In particular embodiments, recombinant receptors include several distinct subcomponents that allow genetically modified cells to recognize and kill unwanted cells, such as cancer cells. The subcomponents include at least an extracellular component and an intracellular component. The extracellular component includes a binding domain that specifically binds a marker that is preferentially present on the surface of unwanted cells. When the binding domain binds such markers, the intracellular component activates the cell to destroy the bound cell. Recombinant receptors additionally include a transmembrane domain that links the extracellular component to the intracellular component, and other subcomponents that can increase the recombinant receptor's function. For example, the inclusion of a spacer and/or one or more linker sequences can allow the recombinant receptor to have additional conformational flexibility, often increasing the binding domain's ability to bind the targeted cell marker. Exemplary binding domains include antibodies or binding fragments thereof as described elsewhere herein.

In particular embodiments, an eTCR includes a T antigen of the Merkel cell polyomavirus binding domain linked to the C_{α} and/or C_{β} chains of a TCR. A TCR is a heterodimeric fusion protein that typically includes an α and β chain. Each chain includes a variable region (V_{α} and V_{β}) and a constant region (C_{α} and C_{β}). In particular embodiments, an eTCR does not include the native TCR variable region but does include the native TCR constant region. In particular embodiments, the eTCR includes a T antigen of the Merkel cell polyomavirus binding domain as the variable region of the α and β chain. In particular embodiments, eTCR include a C_{α} and/or C_{β} chain sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a known or identified TCR C_{α} or C_{β}.

The intracellular component of a CAR includes an effector domain that is responsible for activation of the cell in which the CAR is expressed. The term “effector domain” is thus meant to include any portion of the intracellular domain sufficient to transduce an activation signal. An effector domain can directly or indirectly promote a biological or physiological response in a cell when receiving the appropriate signal. In certain embodiments, an effector domain is part of a protein or protein complex that receives a signal when bound, or it binds directly to a target molecule, which triggers a signal from the intracellular effector domain. An effector domain may directly promote a cellular response when it contains one or more signaling domains or motifs, such as an immunoreceptor tyrosine-based activation motif (ITAM). In other embodiments, an effector domain will indirectly promote a cellular response by associating with one or more other proteins that directly promote a cellular response, such as co-stimulatory domains.

Effector domains can provide for activation of at least one function of a modified cell upon binding to the cellular marker expressed by a T antigen-expressing cell. Activation of the modified cell can include one or more of differentiation, proliferation and/or activation or other effector functions. In particular embodiments, an effector domain can include an intracellular signaling component including a T cell receptor and a co-stimulatory domain which can include the cytoplasmic sequence from co-receptor or co-stimulatory molecule.

An effector domain can include one, two, three or more intracellular signaling domains (e.g., receptor signaling domains, cytoplasmic signaling sequences), co-stimulatory domains, or combinations thereof. Exemplary effector domains include signaling and stimulatory domains selected from: 4-1BB (CD137), CARD11, CD3γ, CD3δ, CD3ϵ, CD3ζ, CD27, CD28, CD79A, CD79B, DAP10, FcRα, FcRβ (FcϵR1b), FcRγ, Fyn, HVEM (LIGHTR), ICOS, LAG3, LAT, Lck, LRP, NKG2D, NOTCH1, pTa, PTCH2, OX40, ROR2, Ryk, SLAMF1, SIp76, TCRα, TCRβ, TRIM, Wnt, Zap70, or any combination thereof. In particular embodiments, exemplary intracellular effector domains include signaling and co-stimulatory domains selected from: CD86, FcγRIIa, DAP12, CD30, CD40, PD-1, lymphocyte function-associated antigen-1 (LFA-1), CD2, CD7, LIGHT, NKG2C, B7-H3, a ligand that specifically binds with CD83, CDS, ICAM-1, GITR, BAFFR, SLAMF7, NKp80 (KLRF1), CD127, CD160, CD19, CD4, CD8α, CD8β, IL2Rβ, IL2Rγ, IL7Rα, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, NKp46, TLR1, TLR2, TLR3, TLR4, TLR5, TLR6, TLR7, TLR8, or TLR9. In particular embodiments, the effector domain includes a CD3ζ signaling domain.

Intracellular signaling domains that act in a stimulatory manner may include iTAMs. Examples of iTAMs including primary cytoplasmic signaling sequences include those derived from CD3γ, CD3δ, CD3ϵ, CD3ζ, CD5, CD22, CD66d, CD79a, CD79b, and common FcRγ (FCER1G), FcγRIIa, FcRβ (Fcϵ Rib), DAP10, and DAP12. In particular embodiments, variants of CD3ζ retain at least one, two, three, or all ITAM regions.

In particular embodiments, an effector domain includes a cytoplasmic portion that associates with a cytoplasmic signaling protein, wherein the cytoplasmic signaling protein is a lymphocyte receptor or signaling domain thereof, a protein including a plurality of ITAMs, a co-stimulatory domain, or any combination thereof.

Additional examples of intracellular signaling domains include the cytoplasmic sequences of the CD3ζ chain, and/or co-receptors that act in concert to initiate signal transduction following binding domain engagement.

A co-stimulatory domain is a domain whose activation can be required for an efficient lymphocyte response to cellular marker binding. Some molecules are interchangeable as intracellular signaling domains or co-stimulatory domains. Examples of costimulatory domains include CD27, CD28, 4-1BB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83. For example, CD27 co-stimulation has been demonstrated to enhance expansion, effector function, and survival of human CAR T cells in vitro and augments human T cell persistence and anti-cancer activity in vivo (Song et al., Blood. 2012; 119(3):696-706). Further examples of such co-stimulatory domain molecules include CDS, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CDlld, ITGAE, CD103, ITGAL, CDlla, ITGAM, CDI Ib, ITGAX, CDlIc, ITGBI, CD29, ITGB2, CD18, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a. In particular embodiments, the co-stimulatory domain includes a 4-1BB signaling domain. In particular embodiments, the co-stimulatory domain includes a CD28 and 4-1BB signaling domain.

In particular embodiments, the nucleic acid sequences encoding the intracellular component include a CD3ζ encoding sequence (SEQ ID NOs: 6 or 7) and a variant of the 4-1BB signaling encoding sequence (SEQ ID NOs: 11, 12, or 13). In particular embodiments, the amino acid sequence of the intracellular component includes a variant of CD3ζ (SEQ ID NOs: 3, 4, or 5) and a portion of the 4-1BB (SEQ ID NO: 8, 9, or 10) intracellular component.

In particular embodiments, the intracellular component includes (i) all or a portion of the signaling domain of CD3ζ, (ii) all or a portion of the signaling domain of 4-1 BB, (iii) all or a portion of the signaling domain of CD28 signaling domain, (iv) all or a portion of the signaling domain of CD3 and 4-1 BB, (v) all or a portion of the signaling domain of CD3ζ and CD28, or (vi) or all or a portion of the signaling domain of CD3ζ, 4-1BB, and CD28.

Intracellular components may also include one or more of a protein of a Wnt signaling pathway (e.g., LRP, Ryk, or ROR2), NOTCH signaling pathway (e.g., NOTCH1, NOTCH2, NOTCH3, or NOTCH4), Hedgehog signaling pathway (e.g., PTCH or SMO), receptor tyrosine kinases (RTKs) (e.g., epidermal growth factor (EGF) receptor family, fibroblast growth factor (FGF) receptor family, hepatocyte growth factor (HGF) receptor family, insulin receptor (IR) family, platelet-derived growth factor (PDGF) receptor family, vascular endothelial growth factor (VEGF) receptor family, tropomycin receptor kinase (Trk) receptor family, ephrin (Eph) receptor family, AXL receptor family, leukocyte tyrosine kinase (LTK) receptor family, tyrosine kinase with immunoglobulin-like and EGF-like domains 1 (TIE) receptor family, receptor tyrosine kinase-like orphan (ROR) receptor family, discoidin domain (DDR) receptor family, rearranged during transfection (RET) receptor family, tyrosine-protein kinase-like (PTK7) receptor family, related to receptor tyrosine kinase (RYK) receptor family, or muscle specific kinase (MuSK) receptor family); G-protein-coupled receptors, GPCRs (Frizzled or Smoothened); serine/threonine kinase receptors (BMPR or TGFR); or cytokine receptors (IL1R, IL2R, IL7R, or IL15R).

As indicated, a transmembrane domain within a CAR serves to connect the extracellular component and intracellular component through the cell membrane. The transmembrane domain can anchor the expressed molecule in the modified cell's membrane.

The transmembrane domain can be derived either from a natural and/or a synthetic source. When the source is natural, the transmembrane domain can be derived from any membrane-bound or transmembrane protein. Transmembrane domains can include at least the transmembrane region(s) of the α, β or ζ chain of a T-cell receptor, CD28, CD27, CD3 epsilon, CD45, CD4, CD5, CD8, CD9, CD16, CD22; CD33, CD37, CD64, CD80, CD86, CD134, CD137 CD154, Toll-like receptor 1 (TLR1), TLR2, TLR3, TLR4, TLR5, TLR6, TLR7, TLR8, and TLR9. In particular embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7R a, ITGA1, VLA1, CD49a, ITGA4, IA4, CD49D, ITGA6, VLA-6, CD49f, ITGAD, CDI Id, ITGAE, CD103, ITGAL, CDI Ia, ITGAM, CDI Ib, ITGAX, CDI Ic, ITGB1, CD29, ITGB2, CD18, ITGB7, TNFR2, DNAM1(CD226), SLAMF4 (CD244, 2B4), CD84, CD96 (Tactile), CEACAM1, CRT AM, Ly9(CD229), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, or NKG2C. In particular embodiments, a variety of human hinges can be employed as well including the human Ig (immunoglobulin) hinge (e.g., an IgG4 hinge, an IgD hinge), a GS linker (e.g., a GS linker described herein), a KIR2DS2 hinge or a CD8a hinge. In particular embodiments, the CAR includes a CD28 transmembrane domain. It has been shown that a CD28 transmembrane domain reduces the antigen-threshold for second-generation 4-1 BB CAR T cell activation.

In particular embodiments, a transmembrane domain has a three-dimensional structure that is thermodynamically stable in a cell membrane, and generally ranges in length from 15 to 30 amino acids. The structure of a transmembrane domain can include an α helix, a β barrel, a β sheet, a β helix, or any combination thereof.

A transmembrane domain can include one or more additional amino acids adjacent to the transmembrane region, e.g., one or more amino acid within the extracellular region of the CAR (e.g., up to 15 amino acids of the extracellular region) and/or one or more additional amino acids within the intracellular region of the CAR (e.g., up to 15 amino acids of the intracellular components). In one aspect, the transmembrane domain is from the same protein that the signaling domain, co-stimulatory domain or the hinge domain is derived from. In another aspect, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins to minimize interactions with other unintended members of the receptor complex. In particular embodiments, the transmembrane domain is encoded by the nucleic acid sequence encoding the CD28 transmembrane domain (SEQ ID NOs: 17, 18, 19, or 20). In particular embodiments, the transmembrane domain includes the amino acid sequence of the CD28 transmembrane domain (SEQ ID NOs: 14, 15, or 16).

Spacers are used to create appropriate distances and/or flexibility from other CAR sub-components. As indicated, in particular embodiments, the length of a spacer is customized for binding targeted (T antigen-expressing) cells and mediating destruction. In particular embodiments, a spacer length can be selected based upon the location of a cellular marker epitope, affinity of a binding domain for the epitope, and/or the ability of the T antigen-binding agent to mediate cell destruction following T antigen binding.

Spacers typically include those having 10 to 250 amino acids, 10 to 200 amino acids, 10 to 150 amino acids, 10 to 100 amino acids, 10 to 50 amino acids, or 10 to 25 amino acids.

In particular embodiments, spacers include all or a portion of an immunoglobulin hinge region. An immunoglobulin hinge region may be a wild-type immunoglobulin hinge region or an altered wild-type immunoglobulin hinge region. In certain embodiments, an immunoglobulin hinge region is a human immunoglobulin hinge region. As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody.

An immunoglobulin hinge region may be an IgG, IgA, IgD, IgE, or IgM hinge region. An IgG hinge region may be an IgG1, IgG2, IgG3, or IgG4 hinge region. Sequences from IgG1, IgG2, IgG3, IgG4 or IgD can be used alone or in combination with all or a portion of a CH2 region; all or a portion of a CH3 region; or all or a portion of a CH2 region and all or a portion of a CH3 region.

In particular embodiments, the spacer includes an IgG4 hinge region; an IgG4 hinge region and an IgG4 CH3 region; or an IgG4 hinge region, an IgG4 CH2 region, and an IgG4 CH3 region.

Other examples of hinge regions that can be used in CAR described herein include the hinge region present in the extracellular regions of type 1 membrane proteins, such as CD8α, CD4, CD28 and CD7, which may be wild-type or variants thereof.

In particular embodiments, a spacer includes a hinge region that includes a type II C-lectin interdomain (stalk) region or a cluster of differentiation (CD) molecule stalk region. A “stalk region” of a type II C-lectin or CD molecule refers to the portion of the extracellular domain (ECD) of the type II C-lectin or CD molecule that is located between the C-type lectin-like domain (CTLD; e.g., similar to CTLD of natural killer cell receptors) and the hydrophobic portion (transmembrane domain). For example, the ECD of human CD94 (GenBank Accession No. AAC50291.1) corresponds to amino acid residues 34-179, but the CTLD corresponds to amino acid residues 61-176, so the stalk region of the human CD94 molecule includes amino acid residues 34-60, which are located between the hydrophobic portion (transmembrane domain) and CTLD (see Boyington et al., Immunity 10:15, 1999; for descriptions of other stalk regions, see also Beavil et al., Proc. Nat'l. Acad. Sci. USA 89:153, 1992; and Figdor et al., Nat. Rev. Immunol. 2:11, 2002). These type II C-lectin or CD molecules may also have junction amino acids (described below) between the stalk region and the transmembrane region or the CTLD. In another example, the 233 amino acid human NKG2A protein (GenBank Accession No. P26715.1) has a hydrophobic portion (transmembrane domain) ranging from amino acids 71-93 and an ECD ranging from amino acids 94-233. The CTLD includes amino acids 119-231 and the stalk region includes amino acids 99-116, which may be flanked by additional junction amino acids. Other type II C-lectin or CD molecules, as well as their extracellular ligand-binding domains, stalk regions, and CTLDs are known in the art (see, e.g., GenBank Accession Nos. NP 001993.2; AAH07037.1; NP 001773.1; AAL65234.1; CAA04925.1; for the sequences of human CD23, CD69, CD72, NKG2A, and NKG2D and their descriptions, respectively).

In particular embodiments, recombinant molecules can include a linker, a control feature, and/or multimerization domain as described below.

(v) Control Features for Recombinantly-Expressed Molecules in Cell-Based Therapies. In particular embodiments, recombinant molecules can include one or more control features A “recombinant molecule” refers to a molecule which is generated using recombinant DNA technology, such as, for example, an antibody expressed by a bacteriophage or yeast expression system. A recombinant molecule can include an antibody, multi-specific binding domain molecule, antibody conjugate, CAR, or eTCR. In particular embodiments, the recombinant molecule is secreted by a cell. In particular embodiments, the recombinant molecule is anchored within a cell membrane such that it is expressed on the surface of the cell.

Tag cassettes can be used to activate, promote proliferation of, detect, enrich for, isolate, track, deplete and/or eliminate genetically modified cells in vitro, in vivo and/or ex vivo. “Tag cassette” refers to a unique synthetic peptide sequence affixed to, fused to, or that is part of a recombinant receptor, to which a cognate binding molecule (e.g., ligand, antibody, or other binding partner) is capable of specifically binding where the binding property can be used to activate, promote proliferation of, detect, enrich for, isolate, track, deplete and/or eliminate the tagged protein and/or cells expressing the tagged protein.

Tag cassettes that bind cognate binding molecules include, for example, His tag (HHHHHH; SEQ ID NO: 178), Flag tag (DYKDDDDK; SEQ ID NO: 179), Xpress tag (DLYDDDDK; SEQ ID NO: 180), Avi tag (GLNDIFEAQKIEWHE; SEQ ID NO: 181), Calmodulin tag (KRRWKKNFIAVSAANRFKKISSSGAL; SEQ ID NO: 182), Polyglutamate tag, HA tag (YPYDVPDYA; SEQ ID NO: 183), Myc tag (EQKLISEEDL; SEQ ID NO: 184), Strep tag (which refers the original STREP® tag (WRHPQFGG; SEQ ID NO: 185), STREP® tag II (WSHPQFEK; SEQ ID NO: 186 (IBA Institut fur Bioanalytik, Germany); see, e.g., U.S. Pat. No. 7,981,632), Softag 1 (SLAELLNAGLGGS; SEQ ID NO: 187), Softag 3 (TQDPSRVG; SEQ ID NO: 188), and V5 tag (GKPIPNPLLGLDST; SEQ ID NO: 189).

Conjugate binding molecules that specifically bind tag cassette sequences disclosed herein are commercially available. For example, His tag antibodies are commercially available from suppliers including Life Technologies, Pierce Antibodies, and GenScript.Flag tag antibodies are commercially available from suppliers including Pierce Antibodies, GenScript, and Sigma-Aldrich. Xpress tag antibodies are commercially available from suppliers including Pierce Antibodies, Life Technologies and GenScript. Avi tag antibodies are commercially available from suppliers including Pierce Antibodies, IsBio, and Genecopoeia. Calmodulin tag antibodies are commercially available from suppliers including Santa Cruz Biotechnology, Abcam, and Pierce Antibodies. HA tag antibodies are commercially available from suppliers including Pierce Antibodies, Cell Signal and Abcam. Myc tag antibodies are commercially available from suppliers including Santa Cruz Biotechnology, Abcam, and Cell Signal. Strep tag antibodies are commercially available from suppliers including Abcam, Iba, and Qiagen.

In particular embodiments, recombinant molecules can include a polynucleotide that encodes a self-cleaving polypeptide, wherein the polynucleotide encoding the self-cleaving polypeptide is located between the polynucleotide encoding the recombinant receptor construct and a polynucleotide encoding a control feature.

Exemplary self-cleaving polypeptides include 2A peptide from porcine teschovirus-1 (P2A), Thosea asigna virus (T2A), equine rhinitis A virus (E2A), foot-and-mouth disease virus (F2A), or variants thereof. Further exemplary nucleic acid and amino acid sequences of 2A peptides are set forth in, for example, Kim et al., (PLOS One 6:e18556 (2011). In particular embodiments, cells are genetically modified to include a self-cleaving polypeptide. In particular embodiments, the self-cleaving polypeptide includes T2A.

Control features may be present in multiple copies in a recombinant molecule or can be expressed as distinct molecules with the use of a skipping element. For example, a recombinant molecule can have one, two, three, four or five tag cassettes and/or one, two, three, four, or five transduction markers could also be expressed. For example, embodiments can include a recombinant receptor construct having two Myc tag cassettes, or a His tag and an HA tag cassette, or a HA tag and a Softag 1 tag cassette, or a Myc tag and a SBP tag cassette. Exemplary transduction markers and cognate pairs are described in U.S. Ser. No. 13/463,247 and elsewhere herein.

One advantage of including at least one control feature in a recombinant molecule is that cells expressing the recombinant molecule administered to a subject can be increased or depleted using the cognate binding molecule to a tag cassette. In certain embodiments, the present disclosure provides a method for depleting a modified cell expressing a recombinant molecule by using an antibody specific for the control feature, using a cognate binding molecule specific for the control feature, or by using a second modified cell expressing a recombinant molecule and having specificity for the control feature. Elimination of modified cells may be accomplished using depletion agents specific for a control feature.

In particular embodiments, a polynucleotide encoding an iCaspase9 construct (iCasp9) may be inserted into a recombinant molecule construct as a suicide switch.

In certain embodiments, modified cells expressing a recombinant molecule may be detected or tracked in vivo by using antibodies that bind with specificity to a control feature (e.g., anti-Tag antibodies), or by other cognate binding molecules that specifically bind the control feature, which binding partners for the control feature are conjugated to a fluorescent dye, radio-tracer, iron-oxide nanoparticle or other imaging agent known in the art for detection by X-ray, CT-scan, MRI-scan, PET-scan, ultrasound, flow-cytometry, near infrared imaging systems, or other imaging modalities (see, e.g., Yu, et al., Theranostics 2:3, 2012).

Thus, modified cells expressing at least one control feature with a recombinant receptor can be, e.g., more readily identified, isolated, sorted, induced to proliferate, tracked, and/or eliminated as compared to a modified cell without a tag cassette.

(vi) Cell Sample Collection and Cell Enrichment. Methods of sample collection and enrichment are known by those skilled in the art. In particular embodiments, cells are derived from humans, for example a patient to be treated. Cells can be derived from cell lines. The cells in some embodiments are obtained from a xenogeneic source, for example, from mouse, rat, non-human primate, or pig.

In some embodiments, immune cells are derived or isolated from samples such as whole blood, peripheral blood mononuclear cells (PBMCs), leukocytes, bone marrow, thymus, tissue biopsy, tumor, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. In particular embodiments, cells from the circulating blood of a subject are obtained, e.g., by apheresis or leukapheresis. The samples, in particular embodiments, contain lymphocytes, including T cells, monocytes, granulocytes, B cells, other nucleated white blood cells, HSC, HPC, HSPC, red blood cells, and/or platelets, and in some aspects contains cells other than red blood cells and platelets and further processing is necessary.

In some embodiments, blood cells collected from a subject are washed, e.g., to remove the plasma fraction and to place the cells in an appropriate buffer or media for subsequent processing steps.

Isolation can include one or more of various cell preparation and separation steps, including separation based on one or more properties, such as size, density, sensitivity or resistance to particular reagents, and/or affinity, e.g., immunoaffinity, to antibodies or other binding partners. In particular embodiments, the isolation is carried out using the same apparatus or equipment sequentially in a single process stream and/or simultaneously. In particular embodiments, the isolation, culture, and/or genetically modifying the different populations is carried out from the same starting material, such as from the same sample.

In particular embodiments, a sample can be enriched for a selected immune cell type by using density-based cell separation methods and related methods. For example, white blood cells can be separated from other cell types in the peripheral blood by lysing red blood cells and centrifuging the sample through a Percoll or Ficoll gradient.

In particular embodiments, a bulk immune cell population can be used that has not been enriched fora particular immune cell type. In particular embodiments, a selected immune cell type can be enriched for and/or isolated based on cell-marker based positive and/or negative selection. In positive selection, cells having bound cellular markers are retained for further use. In negative selection, cells not bound by a capture agent, such as an antibody to a cellular marker are retained for further use. In some examples, both fractions can be retained for a further use.

The separation need not result in 100% enrichment or removal of a particular cell population or cells expressing a particular marker. For example, positive selection of or enrichment for cells of a particular type refers to increasing the number or percentage of such cells but need not result in a complete absence of cells not expressing the marker. Likewise, negative selection, removal, or depletion of cells of a particular type refers to decreasing the number or percentage of such cells but need not result in a complete removal of all such cells.

In some embodiments, an antibody or binding domain for a cellular marker is bound to a solid support or matrix, such as a magnetic bead or paramagnetic bead, to allow for separation of cells for positive and/or negative selection.

In some embodiments, affinity-based selection is via magnetic-activated cell sorting (MACS) (Miltenyi Biotec, Auburn, CA). MACS systems are capable of high-purity selection of cells having magnetized particles attached thereto.

In some embodiments, a cell population described herein is collected and enriched (or depleted) via flow cytometry, in which cells stained for multiple cell surface markers are carried in a fluidic stream. Flow cytometry is a sensitive and powerful analysis approach that uses lasers to individually analyze the fluorescent molecules marking millions of individual cells. By analyzing the combination of fluorescent molecules each cell is marked with, different B cell subtypes can be identified. Flow cytometry can be used to identify B cell subsets and analyze the expression of selected antibodies or gene products by B cells.

In particular embodiments, methods of modifying B cells can include obtaining hematopoietic stem cells (HSC), and/or delivering the genetic constructs to HSC. HSC can refer to a type of stem cell that naturally produces B cells as well as all other cells of the immune system. HSC can be obtained, for example, from cord blood.

In particular embodiments, an immortalized B cell line can be used. In particular embodiments, an immortalized B cell line includes A20 cells.

In particular embodiments, B cells may be obtained from a human subject and obtained B cells or a subset thereof may be modified ex vivo.

In particular embodiments, a selected cell type (e.g., B cells or T cells) is isolated before genetically modifying the select cell. In particular embodiments, cells are isolated after genetic modification. For example, in particular embodiments, B cells can be sorted after genetic modification based on expression of an exogenous light chain. For example, B cells that naturally express a kappa light chain can be modified to express a selected antibody that includes a lambda light chain. B cells that naturally express a lambda light chain can be modified to express a selected antibody that includes a kappa light chain. Sorting based on expression of an exogenous light chain will allow for isolation of only those B cells expressing the selected antibody. In particular embodiments, only those B cells that completely lack surface expression of their endogenous light chain are isolated for formulation and administration to a subject.

(vii) Genetically-Modifying Cell Populations. Desired genes encoding a binding domain molecule (e.g., antibody, CAR, or eTCR) disclosed herein can be introduced into cells by any method known in the art, including transfection, electroporation, microinjection, lipofection, calcium phosphate mediated transfection, infection with a viral or bacteriophage vector including the gene sequences, cell fusion, chromosome-mediated gene transfer, microcell-mediated gene transfer, spheroplast fusion, in vivo nanoparticle-mediated delivery, etc. Numerous techniques are known in the art for the introduction of foreign genes into cells (see e.g., Loeffler and Behr, 1993, Meth. Enzymol. 217:599-618; Cohen, et al., 1993, Meth. Enzymol. 217:618-644; Cline, 1985, Pharmac. Ther. 29:69-92) and may be used, provided that the necessary developmental and physiological functions of the recipient cells are not unduly disrupted. The technique can provide for the stable transfer of the gene to the cell, so that the gene is expressible by the cell and, in certain instances, preferably heritable and expressible by its cell progeny.

The term “gene” refers to a nucleic acid sequence (used interchangeably with polynucleotide or nucleotide sequence) that encodes a binding domain molecule including a T antigen of the Merkel cell polyomavirus binding domain as described herein. This definition includes various sequence polymorphisms, mutations, and/or sequence variants wherein such alterations do not substantially affect the function of the encoded binding domain molecule. The term “gene” may include not only coding sequences but also regulatory regions such as promoters, enhancers, and termination regions. Gene sequences encoding the molecule can be DNA or RNA that directs the expression of the binding domain molecule. These nucleic acid sequences may be a DNA strand sequence that is transcribed into RNA or an RNA sequence that is translated into protein. The sequences can also include degenerate codons of the native sequence or sequences that may be introduced to provide codon preference in a specific cell type. Portions of complete gene sequences are referenced throughout the disclosure as is understood by one of ordinary skill in the art.

Gene sequences encoding the binding domain molecule are provided herein and can also be readily prepared by synthetic or recombinant methods from the relevant amino acid sequences and other description provided herein. In embodiments, the gene sequence encoding any of these sequences can also have one or more restriction enzyme sites at the 5′ and/or 3′ ends of the coding sequence in order to provide for easy excision and replacement of the gene sequence encoding the sequence with another gene sequence encoding a different sequence. In embodiments, the gene sequence encoding the sequences can be codon optimized for expression in mammalian cells.

“Encoding” refers to the property of specific sequences of nucleotides in a gene, such as a cDNA, or an mRNA, to serve as templates for synthesis of other macromolecules such as a defined sequence of amino acids.

Polynucleotide gene sequences encoding more than one portion of an expressed binding domain molecule can be operably linked to each other and relevant regulatory sequences. For example, there can be a functional linkage between a regulatory sequence and an exogenous nucleic acid sequence resulting in expression of the latter. For instance, a promoter is operably linked to a coding sequence if the promoter affects the transcription or expression of the coding sequence.

In particular embodiments, a B cell is genetically modified to express an antibody disclosed herein. In brief, the B cell genome can be targeted for genetic insertion at a target location within an intronic region upstream or downstream of an Ep enhancer element. In particular embodiments, the intronic region includes SEQ ID NO: 1 or SEQ ID NO: 2. In particular embodiments, the B cell is genetically modified to express an antibody as disclosed herein. In particular embodiments, a genetic construct that expressed the antibody is inserted SEQ ID NO: 1 or SEQ ID NO: 2 and includes (i) a promoter; (ii) a signal peptide; (iii) a transgene encoding an entire light chain of the antibody disclosed herein; (iv) a flexible linker or a skipping element; (v) the variable portion of the heavy chain of the antibody disclosed herein; and (vi) a splice junction that results in expression of the B cell's endogenous heavy chain constant region. In these embodiments, expressing the antibody as a single construct overcomes challenges associated with portions of antibodies being encoded by different areas of the endogenous B cell genome. Inclusion of a flexible linker physically links the light chain portion and the heavy chain portion of the expressed selected antibody in a manner that allows them to form a functional unit and at the same time reduces the risk of the antibody portions binding with other potentially expressed antibody chains from the B cell's endogenous genome. Use of a skipping element does not physically link the light chain portion and the heavy chain portion, but their expression in close proximity also results in association to form a functional unit while at the same time reducing the risk of the antibody portions binding with other potentially expressed antibody chains from the B cell's endogenous genome. Inclusion of a splice junction results in the selected antibody including a heavy chain constant region appropriate for the B cell's current activation and/or maturation state. In other words, the selected expressed antibodies can be expressed having any of the B cell's endogenous heavy chain constant regions, and the heavy chain constant region expressed with the selected antibody can naturally change over time. Additional details and alternative embodiments for genetically modifying a B cell to express the antibody disclosed herein can be found in, for example, PCT Pub. No. WO 2019/079772, incorporated herein by reference.

In particular embodiments, targeted genetic modification systems include a targeting element for precise targeting and a cutting element for cutting the targeted genetic site. Guide RNA is one example of a targeting element while various nucleases provide examples of cutting elements. Targeting elements and cutting elements can be separate molecules or linked, for example, by a nanoparticle. In particular embodiments, the targeting elements and/or cutting elements can be associated with a nanoparticle. Herein, “associated with” means functionally linked such that targeted genetic insertion occurs at an intended genomic site. A functional linkage to a nanoparticle can be through covalent linkage, through electrostatic attraction or through an intervening molecule or layer. Alternatively, a targeting element and a cutting element can be linked together into one dual purpose molecule. When insertion of a genetic construct is intended, systems can also include homology-directed repair templates (i.e., homology regions also commonly referred to as homology arms) associated with the genetic constructs. Different gene editing systems can adopt different components and configurations while maintaining the ability to precisely target, cut, and modify selected genomic sites.

The CRISPR (Clustered Regularly Interspaced Short Palindromic Repeats)/Cas (CRISPR-associated protein) nuclease system is an engineered nuclease system used for genetic modification that is based on a bacterial system. Information regarding CRISPR-Cas systems and components thereof are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

Particular embodiments utilize zinc finger nucleases (ZFNs) as gene editing agents. For information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933,113; 6,979,539; 7,013,219; 7,030,215; 7,220,719; 7,241,573; 7,241,574; 7,585,849; 7,595,376; 6,903,185; 6,479,626; US 2003/0232410 and US 2009/0203140 as well as Gaj et al., Nat Methods, 2012, 9(8):805-7; Ramirez et al., Nucl Acids Res, 2012, 40(12):5560-8; Kim et al., Genome Res, 2012, 22(7): 1327-33; Urnov et al., Nature Reviews Genetics, 2010, 11:636-646; Miller, et al., Nature biotechnology 25, 778-785 (2007); Bibikova, et al., Science 300, 764 (2003); Bibikova, et al., Genetics 161, 1169-1175 (2002); Wolfe, et al., Annual review of biophysics and biomolecular structure 29, 183-212 (2000); Kim, et al., Proceedings of the National Academy of Sciences of the United States of America 93, 1156-1160 (1996); and Miller, et al., The EMBO journal 4, 1609-1614 (1985).

Particular embodiments can use transcription activator like effector nucleases (TALENs) as gene editing agents. For information regarding TALENs, see U.S. Pat. Nos. 8,440,431; 8,440,432; 8,450,471; 8,586,363; and 8,697,853; as well as Joung and Sander, Nat Rev Mol Cell Biol, 2013, 14(1):49-55; Beurdeley et al., Nat Commun, 2013, 4: 1762; Scharenberg et al., Curr Gene Ther, 2013, 13(4):291-303; Gaj et al., Nat Methods, 2012, 9(8):805-7; Miller, et al., Nature biotechnology 29, 143-148 (2011); Christian, et al., Genetics 186, 757-761 (2010); Boch, et al., Science 326, 1509-1512 (2009); and Moscou, & Bogdanove, Science 326, 1501 (2009).

Particular embodiments can utilize MegaTALs as gene editing agents. MegaTALs have a sc rare-cleaving nuclease structure in which a TALE is fused with the DNA cleavage domain of a meganuclease. Meganucleases, also known as homing endonucleases, are single peptide chains that have both DNA recognition and nuclease function in the same domain. In contrast to the TALEN, the megaTAL only requires the delivery of a single peptide chain for functional activity.

Particular embodiments can use transposon-based systems as gene editing agents to mediate the integration of a binding domain molecule construct into cells.

Several transposon/transposase systems have been adapted for genetic insertions of heterologous DNA sequences. Examples of such transposases include sleeping beauty (“SB”, e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Toll; Tol2 (e.g., derived from medaka fish); TcBuster (e.g., derived from the red flour beetle Tribolium castaneum), Helraiser, Himar1, Passport, Minos, Ac/Ds, PIF, Harbinger, Harbinger3-DR, HSmar1, and spinON. Transposases and transposon systems are further described in U.S. Pat. Nos. 6,489,458; 7,148,203; 8,227,432; and 9,228,180.

Nanoparticles that result in selective in vivo genetic modification of targeted cell types can be used within the teachings of the current disclosure. In particular embodiments, the nanoparticles can be those described in WO2014153114, WO2017181110, and WO201822672.

Exemplary cell-targeted nanoparticles include a cell targeting ligand on the surface of the nanoparticle wherein the cell targeting ligand results in selective uptake of the nanoparticle by a selected cell type (e.g., B cell or T cell). Exemplary cell targeting ligands for selected cell types include molecules that target cell markers expressed by the selected cell type. The nanoparticle then binds the selected cell type, delivering gene modifying components that result in expression of the selected antibody and gene product.

Exemplary nanoparticles include liposomes (microscopic vesicles including at least one concentric lipid bilayer surrounding an aqueous core), liposomal nanoparticles (a liposome structure used to encapsulate another smaller nanoparticle within its core); and lipid nanoparticles (liposome-like structures that lack the continuous lipid bilayer characteristic of liposomes). Other polymer-based nanoparticles can also be used as well as porous nanoparticles constructed from any material capable of forming a porous network. Exemplary materials include metals, transition metals and metalloids (e.g., lithium, magnesium, zinc, aluminum and silica).

For in vivo delivery and cellular uptake, nanoparticles can have a neutral or negatively-charged coating and a size of 130 nm or less. Dimensions of the nanoparticles can be determined using, e.g., conventional techniques, such as dynamic light scattering and/or electron microscopy.

Another method of genetically modifying a cell population includes vectors. A “vector” is a nucleic acid molecule that is capable of transporting another nucleic acid. Vectors may be, e.g., plasmids (DNA plasmids or RNA plasmids), transposon-based systems, cosmids, bacterial artificial chromosomes, viruses, or phage. An “expression vector” is a vector that is capable of directing the expression of a protein encoded by one or more genes carried by the vector when it is present in the appropriate environment.

“Lentivirus” refers to a genus of retroviruses that are capable of infecting dividing and non-dividing cells. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

A lentiviral vector is a vector derived from at least a portion of a lentivirus genome, including especially a self-inactivating lentiviral vector as provided in Milone et ah, Mol. Ther. 17(8): 1453-1464 (2009). Other examples of lentivirus vectors that may be used in the clinic, include: the LENTIVECTOR® gene delivery technology from Oxford BioMedica, the LENTIMAX™ vector system from Lentigen and the like.

There are a large number of available viral vectors suitable within the current disclosure, including those identified for human gene therapy applications (see Pfeifer and Verma, 2001, Ann. Rev. Genomics Hum. Genet. 2:177). Methods of using retroviral and lentiviral viral vectors and packaging cells for transducing mammalian host cells with viral particles including binding domain molecule transgenes are described in, e.g., U.S. Pat. No. 8,119,772; Walchli, et al., 2011, PLoS One 6:327930; Zhao, et al., 2005, J. Immunol. 174:4415; Engels, et al., 2003, Hum. Gene Ther. 14:1155; Frecha, et al., 2010, Mol. Ther. 18:1748; and Verhoeyen, et al., 2009, Methods Mol. Biol. 506:97. Retroviral and lentiviral vector constructs and expression systems are also commercially available.

In particular embodiments, cells can be genetically modified in vivo or ex vivo.

In particular embodiments, gene product expression can be confirmed using any method known to a person skilled in the art. For example, gene product expression can be confirmed using a transduction marker, a selection cassette, and/or a tag cassette.

Transduction markers, selection cassettes, and tag cassettes can serve to activate, promote proliferation of, detect, enrich for, isolate, track, deplete and/or eliminate the cells expressing the recombinant protein.

Transduction markers are derived from naturally occurring molecules and are often expressed using a skipping element that separates the transduction marker from the rest of the recombinant receptor. Transduction markers may be selected from at least one of a truncated CD19 (tCD19; see Budde et al., Blood 122: 1660, 2013); a truncated human EGFR (tEGFR or EGFRt; see Wang et al., Blood 118: 1255, 2011); an ECD of human CD34; and/or RQR8 which combines target epitopes from CD34 (see Fehse et al, Mol. Therapy 1(5 Pt 1); 448-456, 2000) and CD20 antigens (see Philip et al, Blood 124: 1277-1278). In particular embodiments, cells are genetically modified to express EGFRt.

Selection cassettes provide for either positive selection or negative selection of a desired cell population. Negative selection is when several cell types are removed, leaving the cell type of interest. Positive selection involves targeting the desired cell population to only retain desired cells. The selection cassette can encode proteins that (a) confer resistance to antibiotics or other toxins, (b) complement auxotrophic deficiencies, or (c) supply critical nutrients not available from complex media, e.g., the gene encoding D-alanine racemase for Bacilli. Any number of selection systems may be used to recover transformed cells. In particular embodiments, a positive selection cassette includes resistance genes to neomycin, hygromycin, ampicillin, puromycin, phleomycin, zeomycin, blasticidin, or viomycin. In particular embodiments, a selection cassette includes the DHFR (dihydrofolate reductase) gene or DHFR double mutant (DHFRdm) gene providing resistance to methotrexate (MTX), the MGMT P140K gene responsible for the resistance to 0⁶BG/BCNU, the HPRT (Hypoxanthine phosphoribosyl transferase) gene responsible for the transformation of specific bases present in the HAT selection medium (aminopterin, hypoxanthine, thymidine) or other genes for detoxification with respect to some drugs.

A tag cassette can also be used to confirm gene product expression within a cell population. In particular embodiments, the tag cassette is expressed as part of a recombinant protein expressed by the genetically modified cell. In particular embodiments, the tag cassette can be detected when the tag cassette binds its cognate binding molecule. In particular embodiments, the cognate binding molecule can be linked to a detectable label. Tag cassettes and detectable labels are described in further detail elsewhere herein.

In particular embodiments, a sample can be extracted from subject tissue or blood. Gene product expression within the sample can be measured using immunoblotting, immunoprecipitation, immunofluorescence, chemiluminescence, electro-chemiluminescence (ECL), enzyme-linked immunoassays (ELISA), chromatography, spectroscopy, capillary electrophoresis, mass spectrometry, and/or any other qualitative or quantitative assay known in the art.

In particular embodiments, the genetically modified cells can be assessed for surface expression of the binding domain molecule. In particular embodiments, at least 50%, at least 55%, at least 60%, at least 65%, at least 70%, at least 75%, at least 80%, at least 85%, at least 90%, at least 95%, or at least 99% of the genetically modified cells express a detectable level of the binding domain molecule.

(viii) Cell-Based Formulations. After genetic modification, cells can be prepared into a formulation. Genetically modified cells can be harvested from a culture medium, washed, and concentrated into a carrier in a therapeutically-effective amount, thus preparing a formulation. In particular embodiments, a modifying formulation includes a nucleic acid for genetically modifying the cell population (e.g., nucleic acids encapsulated in nanoparticles and/or targeted genetic modification systems). In particular embodiments, formulations and/or modifying formulations include genetically modified cells and/or nucleic acids to modify cells within a pharmaceutically acceptable carrier.

Exemplary carriers include saline, buffered saline, physiological saline, water, Hanks' solution, Ringer's solution, Normosol-R (Abbott Labs), PLASMA-LYTE A® (Baxter Laboratories, Inc., Morton Grove, IL), glycerol, ethanol, and combinations thereof.

In particular embodiments, carriers can be supplemented with human serum albumin (HSA) or other human serum components or fetal bovine serum. In particular embodiments, a carrier for infusion includes buffered saline with 5% hyaluronic acid sodium salt (HAS) or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

In particular embodiments, cells are in a formulation volume of a liter or less, 500 ml or less, 250 ml or less, or 100 ml or less. Hence, the density of administered cells is typically greater than 10⁴ cells/ml, 10⁵ cells/ml, 10⁶ cells/ml, 10⁷ cells/ml, or 10⁸ cells/ml.

Any formulation disclosed herein can advantageously include any other pharmaceutically acceptable carriers which include those that do not produce significantly adverse, allergic, or other untoward reactions that outweigh the benefit of administration. Exemplary pharmaceutically acceptable carriers are disclosed in Remington's Pharmaceutical Sciences, 18th Ed. Mack Printing Company, 1990. Moreover, formulations can be prepared to meet sterility, pyrogenicity, general safety, and purity standards as required by U.S. FDA Office of Biological Standards and/or other relevant foreign regulatory agencies.

(ix) Multi-Specific Binding Domain Molecules. A binding domain molecule refers to a molecule having a binding domain as disclosed herein. In particular embodiments, a binding domain molecule includes an antibody, an antibody fragment, and a recombinant receptor. In particular embodiments, antibodies and antibody fragments are described elsewhere herein.

A multi-specific binding domain molecule binds at least two epitopes wherein at least one of the epitopes is located on the T antigen of the Merkel cell polyomavirus. Multi-specific binding domain molecules include bispecific antibodies, tri-specific antibodies, and so on.

Bispecific antibodies can be prepared as full-length antibodies or antibody fragments (for example, F(ab′)₂ bispecific antibodies). For example, WO 1996/016673 describes a bispecific anti-ErbB2/anti-Fc gamma RIII antibody; U.S. Pat. No. 5,837,234 describes a bispecific anti-ErbB2/anti-Fc gamma RI antibody; WO 1998/002463 describes a bispecific anti-ErbB2/Fc alpha antibody; and U.S. Pat. No. 5,821,337 describes a bispecific anti-ErbB2/anti-CD3 antibody. In particular embodiments, a bispecific antibody can be in the form of a Bispecific T-cell Engaging (BiTE®) antibody.

Some additional exemplary bispecific antibodies have two heavy chains (each having three heavy chain CDRs, followed by (N-terminal to C-terminal) a CH1 domain, a hinge, a CH2 domain, and a CH3 domain), and two immunoglobulin light chains that confer antigen-binding specificity through association with each heavy chain. However, as indicated, additional architectures are envisioned, including bi-specific antibodies in which the light chain(s) associate with each heavy chain but do not (or minimally) contribute to antigen-binding specificity, or that can bind one or more of the epitopes bound by the heavy chain antigen-binding regions, or that can associate with each heavy chain and enable binding of one or both of the heavy chains to one or both epitopes.

scFv dimers or diabodies may be used, rather than whole antibodies. Diabodies and scFv can be constructed without an Fc region, using only variable domains (usually including the variable domain components from both light and heavy chains of the source antibody), potentially reducing the effects of anti-idiotypic reaction. Other forms of bispecific antibodies include the single chain “Janusins” described in Traunecker et al., (Embo Journal, 10, 3655-3659, 1991).

Bispecific antibodies with extended half-lives are described in, for example, U.S. Pat. No. 8,921,528 and US Patent Publication No. 2014/0308285.

Methods for making bispecific antibodies are known in the art. For example, traditional production of full-length bispecific antibodies is based on the co-expression of two immunoglobulin heavy chain-light chain pairs, where the two chains have different specificities (see, for example, Millstein et al., Nature 305:37-39, 1983). Similar procedures are disclosed in, for example, WO 1993/008829, Traunecker et al., EMBO J. 10:3655-3659, 1991 and Holliger & Winter, Current Opinion Biotechnol. 4, 446-449 (1993).

In particular embodiments, bispecific antibodies can be prepared using chemical linkage. For example, Brennan et al., (Science 229: 81, 1985) describes a procedure wherein intact antibodies are proteolytically cleaved to generate F(ab′)2 fragments. These fragments are reduced in the presence of the dithiol complexing agent, sodium arsenite, to stabilize vicinal dithiols and prevent intermolecular disulfide formation. The Fab′fragments generated then are converted to thionitrobenzoate (TNB) derivatives. One of the Fab′-TNB derivatives then is reconverted to the Fab′-thiol by reduction with mercaptoethylamine and is mixed with an equimolar amount of the other Fab′-TNB derivative to form the bispecific antibody.

In particular embodiments, binding domains disclosed herein can be used to create bi-tri, (or more) specific immune cell engaging antibody constructs. The immune cell engaging antibody constructs can engage, for example, T-cells, B cells, natural killer (NK) cells, NK-T cells, monocytes/macrophages, lymphocytes, hematopoietic stem cells (HSCs), hematopoietic progenitor cells (HPC), and/or a mixture of HSC and HPC (i.e., HSPC). In particular embodiments, B cells are targeted for activation by immune cell engaging antibody constructs. Examples of such B cell activating epitopes are on B cell antigens including CD40, ICOS, IL21R, and IL4R.

In particular embodiments, the immune cell engaging antibody constructs engage T-cells. T-cell activation can be mediated by two distinct signals: those that initiate antigen-dependent primary activation and provide a T-cell receptor like signal (primary cytoplasmic signaling sequences) and those that act in an antigen independent manner to provide a secondary or co-stimulatory signal (secondary cytoplasmic signaling sequences). I-AMS disclosed herein can target any T-cell activating epitope that upon binding induces T-cell activation. Examples of such T-cell activating epitopes are on T-cell markers including CD2, CD3, CD7, CD27, CD28, CD30, CD40, CD83, 4-1 BB (CD 137), OX40, lymphocyte function-associated antigen-1 (LFA-1), LIGHT, NKG2C, and B7-H3. Binding domains that bind T-cell markers are known in the art.

In particular embodiments macrophages are targeted for localized activation by I-AMS. Macrophages are a type of leukocyte (or white blood cell) that can engulf and digest cells, cellular debris, and/or foreign substances in a process known as phagocytosis.

The I-AMS can be designed to bind to a protein expressed on the surface of macrophages. Examples of activating proteins expressed on the surface of macrophages (and their precursors, monocytes) include CD11b, CD11c, CD64, CD68, CD119, CD163, CD206, CD209, F4/80, IFGR2 Toll-like receptors (TLRs) 1-9, IL-4Ra, and MARCO. Commercially available antibodies that bind to proteins expressed on the surface of macrophages include M1/70, which binds and activates CD11b (available from BioLegend®); KP1, which binds and activates CD68 (available from ABCAM®, Cambridge, United Kingdom); and ab87099, which binds and activates CD163 (available from ABCAM®).

In particular embodiments, I-AMS can target a pathogen recognition receptor (PRR). PRRs are proteins or protein complexes that recognize a danger signal and activate and/or enhance the innate immune response. Examples of PRRs include the TLR4/MD-2 complex, which recognizes gram negative bacteria; Dectin-1 and Dectin-2, which recognize mannose moieties on fungus and other pathogens; TLR2/TLR6 or TLR2/TLR1 heterodimers, which recognize gram positive bacteria; TLR5, which recognizes flagellin; and TLR9 (CD289), which recognizes CpG motifs in DNA. In particular embodiments, I-AMS can bind and activate TLR4/MD-2, Dectin-1, Dectin-2, TRL2/TLR6, TLR2/TLR1, TLR5, and/or TLR9.

In particular embodiments, I-AMS can target the complement system. The complement system refers to an immune pathway that is induced by antigen-bound antibodies and involves signaling of complement proteins, resulting in immune recognition and clearance of the antibody-coated antigens.

Binding domains of I-AMS and other engineered formats described herein may be joined through a linker. A linker is an amino acid sequence which can provide flexibility and room for conformational movement between the binding domains of a I-AM. Any appropriate linker may be used.

Examples of linkers can be found in Chen et al., (Adv Drug Deliv Rev. 2013 Oct. 15; 65(10): 1357-1369) and described elsewhere herein. Linkers can be flexible, rigid, or semi-rigid, depending on the desired functional domain presentation to a target.

Cytolytic properties of I-AMS molecules can be confirmed in comparative in vitro assays. Briefly, for cell line experiments, target cells can be incubated in 96-well round bottom plates at 5-10,000 cells/well containing increasing concentrations of the various I-AMS antibodies with/without healthy donor T-cells (used at an E:T cell ratio of 1:1 and 3:1). After 48 hours, cell numbers and drug-induced cytotoxicity, using 4′,6-diamidino-2-phenylindole (DAPI) to detect non-viable cells, can be determined by flow cytometry. In experiments where healthy donor T-cells are added, cells can be identified by forward/side scatter properties and negativity for CellVue Burgundy dye. Experiments can include technical duplicates.

In particular embodiments including I-AMS constructs, T-cell activating epitope binding domains include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the V_{α}, V_{β}, C_{α}, or C_{β} of a known TCR. An insertion, deletion or substitution may be anywhere in a V_{α}, V_{β}, C_{α}, or C_{β} region, including at the amino- or carboxy-terminus or both ends of these regions, provided that each CDR includes zero changes or at most one, two, or three changes and provided a binding domain including a modified V_{α}, V_{β}, C_{α}, or C_{β} region can still specifically bind its target with an affinity similar to wild type.

Tri-specific antibodies are artificial proteins that simultaneously bind to three different types of antigens, wherein at least one of the antigens is the T antigen of the Merkel cell polyomavirus. Tri-specific antibodies are described in, for example, WO2016/105450, WO 2010/028796; WO 2009/007124; WO 2002/083738; US 2002/0051780; and WO 2000/018806.

In some embodiments, multi-specific binding domain molecules can be prepared using multimerization domains as described elsewhere herein.

(x) Antibody Conjugates. Antibody conjugates include a T antigen of the Merkel cell polyomavirus binding domain conjugated to a therapeutic or diagnostic agent. In particular embodiments, antibody conjugates include immunotoxins, antibody-drug conjugates (ADCs), detectable label conjugates, and radioisotope conjugates.

In particular embodiments, the antibodies can be formed as immunotoxins. Immunotoxins include a T antigen of the Merkel cell polyomavirus binding domain disclosed herein conjugated to one or more cytotoxins (e.g., protein toxins, enzymatically active toxins of bacterial, fungal, plant, or animal origin, or fragments thereof). A toxin can be any agent that is detrimental to cells. Frequently used plant toxins are divided into two classes: (1) holotoxins (or class II ribosome inactivating proteins), such as ricin, abrin, mistletoe lectin, and modeccin, and (2) hemitoxins (class I ribosome inactivating proteins), such as pokeweed antiviral protein (PAP), saporin, Bryodin 1, bouganin, and gelonin. Commonly used bacterial toxins include diphtheria toxin (DT) and Pseudomonas exotoxin (PE). Kreitman, Current Pharmaceutical Biotechnology 2:313-325 (2001). The toxin may be obtained from essentially any source and can be a synthetic or a natural product.

Immunotoxins with multiple (e.g., four) cytotoxins per binding domain can be prepared by partial reduction of the binding domain with an excess of a reducing reagent such as dithiothreitol (DTT) or tris(2-carboxyethyl)phosphine (TCEP) at 37° C. for 30 min, then the buffer can be exchanged by elution through SEPHADEX G-25 resin with 1 mM DTPA (diethylene triamine penta-acetic acid) in Dulbecco's phosphate-buffered saline (DPBS). The eluent can be diluted with further DPBS, and the thiol concentration of the binding domain can be measured using 5,5′-dithiobis(2-nitrobenzoic acid) [Ellman's reagent]. An excess, for example 5-fold, of the linker-cytotoxin conjugate can be added at 4° C. for 1 hr, and the conjugation reaction can be quenched by addition of a substantial excess, for example 20-fold, of cysteine. The resulting immunotoxin mixture can be purified on SEPHADEX G-25 equilibrated in PBS to remove unreacted linker-cytotoxin conjugate, desalted if desired, and purified by size-exclusion chromatography. The resulting immunotoxin can then be sterile filtered, for example, through a 0.2 μm filter, and can be lyophilized if desired for storage.

Antibody-drug conjugates (ADC) allow for the targeted delivery of a drug moiety to a T antigen-expressing cell, and, in particular embodiments intracellular accumulation therein, where systemic administration of unconjugated drugs may result in unacceptable levels of toxicity to normal cells (Polakis P. (2005) Current Opinion in Pharmacology 5:382-387).

In particular embodiments, ADC refer to targeted chemotherapeutic molecules which combine properties of both antibodies and cytotoxic drugs by targeting potent cytotoxic drugs to antigen-expressing cancer cells (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169; Chari, R. V. (2008) Acc. Chem. Res. 41:98-107). See also Kamath & lyer (Pharm Res. 32(11): 3470-3479, 2015), which describes considerations for the development of ADCs.

The drug moiety (D) of the ADC may include any compound, moiety or group that has a cytotoxic or cytostatic effect. Drug moieties may impart their cytotoxic and cytostatic effects by mechanisms including tubulin binding, DNA binding or intercalation, and inhibition of RNA polymerase, protein synthesis, and/or topoisomerase. Exemplary drugs include actinomycin D, anthracycline, auristatin, calicheamicin, camptothecin, CC1065, colchicin, cytochalasin B, daunorubicin, 1-dehydrotestosterone, dihydroxy anthracinedione, dolastatin, doxorubicin, duocarmycin, elinafide, emetine, ethidium bromide, etoposide, gramicidin D, glucocorticoids, lidocaine, maytansinoid (including monomethyl auristatin E [MMAE]; vedotin), mithramycin, mitomycin, mitoxantrone, nemorubicin, PNU-159682, procaine, propranolol, puromycin, pyrrolobenzodiazepine (PBD), taxane, taxol, tenoposide, tetracaine, trichothecene, vinblastine, vinca alkaloid, vincristine, and stereoisomers, isosteres, analogs, and derivatives thereof that have cytotoxic activity.

The drug may be obtained from essentially any source; it may be synthetic or a natural product isolated from a selected source, e.g., a plant, bacterial, insect, mammalian or fungal source. The drug may also be a synthetically modified natural product or an analogue of a natural product.

ADC compounds of the disclosure include those that target T antigen-expressing cells. In particular embodiments, the ADC compounds include an antibody conjugated, i.e., covalently attached, to the drug moiety. In particular embodiments, the antibody is covalently attached to the drug moiety through a linker. A linker can include any chemical moiety that is capable of linking an antibody, antibody fragment (e.g., antigen binding fragments) or functional equivalent to another moiety, such as a drug moiety. Linkers can be susceptible to cleavage (cleavable linker), such as, acid-induced cleavage, photo-induced cleavage, peptidase-induced cleavage, esterase-induced cleavage, and disulfide bond cleavage, at conditions under which the compound or the antibody remains active. Alternatively, linkers can be substantially resistant to cleavage (e.g., stable linker or noncleavable linker). In some aspects, the linker is a procharged linker, a hydrophilic linker, or a dicarboxylic acid-based linker. The ADCs selectively deliver an effective dose of a drug to cancer cells whereby greater selectivity, i.e., a lower efficacious dose, may be achieved while increasing the therapeutic index (“therapeutic window”).

To prepare ADCs, linker-cytotoxin conjugates can be made by conventional methods analogous to those described by Doronina et al., (Bioconjugate Chem. 17: 114-124, 2006). Antibody-drug conjugates with multiple (e.g., four) drugs per antibody can be prepared by partial reduction of the antibody with an excess of a reducing reagent such as dithiothreitol (DTT) or tris(2-carboxyethyl)phosphine (TCEP) at 37° C. for 30 min, then the buffer can be exchanged by elution through SEPHADEX G-25 resin with 1 mM DTPA in Dulbecco's phosphate-buffered saline (DPBS). The eluent can be diluted with further DPBS, and the thiol concentration of the antibody can be measured using 5,5′-dithiobis(2-nitrobenzoic acid) [Ellman's reagent]. An excess, for example 5-fold, of the linker-cytotoxin conjugate can be added at 4° C. for 1 hr, and the conjugation reaction can be quenched by addition of a substantial excess, for example 20-fold, of cysteine. The resulting ADC mixture can be purified on SEPHADEX G-25 equilibrated in PBS to remove unreacted linker-cytotoxin conjugate, desalted if desired, and purified by size-exclusion chromatography. The resulting ADC can then be sterile filtered, for example, through a 0.2 μm filter, and can be lyophilized if desired for storage.

Detectable label conjugates include a T antigen of the Merkel cell polyomavirus binding domain linked to a detectable label. In particular embodiments, a detectable label includes a fluorescent label, a chemiluminescent label, a spectral colorimetric label, an enzymatic label, or an affinity tag.

Fluorescent labels can include any suitable label or detectable group detectable by, for example, optical, spectroscopic, photochemical, biochemical, immunochemical, electrical, optical or chemical means.

Fluorescent labels can be particularly useful in cell staining, identification, and isolation uses. Exemplary fluorescent labels include blue fluorescent proteins (e.g. eBFP, eBFP2, Azurite, mKalama1, GFPuv, Sapphire, T-sapphire); cyan fluorescent proteins (e.g. eCFP, Cerulean, CyPet, AmCyanl, Midoriishi-Cyan, mTurquoise); green fluorescent proteins (e.g. GFP, GFP-2, tagGFP, turboGFP, EGFP, Emerald, Azami Green, Monomeric Azami Green (mAzamigreen)), CopGFP, AceGFP, avGFP, ZsGreenl, Oregon Green™ (Thermo Fisher Scientific)); Luciferase; orange fluorescent proteins (mOrange, mKO, Kusabira-Orange, Monomeric Kusabira-Orange, mTangerine, tdTomato); red fluorescent proteins (mKate, mKate2, mPlum, DsRed monomer, mCherry, mRuby, mRFP1, DsRed-Express, DsRed2, DsRed-Monomer, HcRed-Tandem, HcRedl, AsRed2, eqFP611, mRaspberry, mStrawberry, Jred, Texas Red™ (Thermo Fisher Scientific)); far red fluorescent proteins (e.g., mPlum and mNeptune); yellow fluorescent proteins (e.g., YFP, eYFP, Citrine, SYFP2, Venus, YPet, PhiYFP, ZsYellowl); and tandem conjugates.

In particular embodiments, a chemiluminescent label includes lucigenin, luminol, luciferin, isoluminol, theromatic acridinium ester, imidazole, acridinium salt, or oxalate ester. In particular embodiments, spectral colorimetric label includes colloidal gold. In particular embodiments, the enzymatic label includes malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase, or acetylcholinesterase.

In particular embodiments, an affinity tag includes a His tag, Flag tag, Xpress tag, Avi tag, Calmodulin tag, Polyglutamate tag, HA tag, Myc tag, Strep tag (which refers the original STREP® tag, STREP® tag II ((IBA Institut fur Bioanalytik, Germany); see, e.g., U.S. Pat. No. 7,981,632), Softag 1, Softag 3, and V5 tag as described elsewhere herein.

Radioisotope conjugates include a T antigen of the Merkel cell polyomavirus binding domain linked to a radioisotope for use in nuclear medicine. Nuclear medicine refers to the diagnosis and/or treatment of conditions by administering radioactive isotopes (radioisotopes or radionuclides) to a subject. Therapeutic nuclear medicine is often referred to as radiation therapy or radioimmunotherapy (RIT).

Examples of radioactive isotopes that can be conjugated to antibodies of the present disclosure include iodine-131, arsenic-72, arsenic-74, iodine-131, indium-111, yttrium-90, and lutetium-177, as well as alpha-emitting radionuclides such as astatine-211, actinium-225, bismuth-212 or bismuth-213. Methods for preparing radioimmunoconjugates are established in the art. Examples of radioimmunoconjugates are commercially available, including Zevalin™ (DEC Pharmaceuticals), and similar methods can be used to prepare radioimmunoconjugates using the antibodies of the disclosure.

Examples of radionuclides that are useful for radiation therapy include ²²⁵Ac and ²²⁷Th. ²²⁵Ac is a radionuclide with the half-life of ten days. As ²²⁵Ac decays the daughter isotopes ²²¹Fr, ²¹³Bi, and ²⁰⁹Pb are formed. ²²⁷Th has a half-life of 19 days and forms the daughter isotope ²²³Ra.

Additional examples of useful radioisotopes include ²²⁸Ac, ¹¹¹Ag, ¹²⁴Am, ⁷⁴As, ²¹¹At, ²⁰⁹At, ¹⁹⁴Au, ¹²⁸Ba, ⁷Be, ²⁰⁶Bi, ²⁴⁵Bk, ²⁴⁶Bk, ⁷⁶Br, ¹¹C, ⁴⁷Ca, ²⁵⁴Cf, ²⁴²Cm, ⁵¹Cr, ⁶⁷Cu, ¹⁵³Dy, ¹⁵⁷Dy, ¹⁵⁹Dy, ¹⁶⁵Dy, ¹⁶⁶Dy, ¹⁷¹Er, ²⁵⁰Es, ²⁵⁴Es, ¹⁴⁷Eu, ¹⁵⁷Eu, ⁵²Fe, ⁵⁹Fe, ²⁵¹Fm, ²⁵²Fm, ²⁵³Fm, ⁶⁶Ga, ⁷²Ga, ¹⁴⁶Gd, ¹⁵³Gd. ⁶⁸Ge, ¹⁷⁰Hf, ¹⁷¹Hf, ¹⁹³Hg, ¹⁹³mHg, ¹⁶⁰mHo, ¹³⁰I, ¹³¹I, ¹³⁵I, ¹¹⁴mIn, ¹⁸⁵Ir, ⁴²K, ⁴³K, ⁷⁶Kr, ⁷⁹Kr, ⁸¹mKr, ¹³²La, ²⁶²Lr ¹⁶⁹Lu, ¹⁷⁴mLu, ¹⁷⁶mLu, ²⁵⁷Md, ²⁶⁰Md, ²⁸Mg, ⁵²Mn, ⁹⁰Mo, ²⁴Na, ⁹⁵Nb, ¹³⁸Nd, ⁵⁷Ni, ⁶⁶Ni, ²³⁴Np, ¹⁵O, ¹⁸²Os, ¹⁸⁹mOs, ¹⁹¹Os, ³²P, ²⁰¹Pb, ¹⁰¹Pd, ¹⁴³Pr, ¹⁹¹Pt, ²⁴³Pu ²²⁵Ra, ⁸¹Rb, ¹⁸⁸Re, ¹⁰⁵Rh, ²¹¹Rn, ¹⁰³Ru, ³⁵S, ⁴⁴Sc, ⁷²Se, ¹⁵³Sm, ¹²⁵Sn, ⁹¹Sr, ¹⁷³Ta, ¹⁵⁴Tb, ¹²⁷Te, ²³⁴Th, ⁴⁵Ti, ¹⁶⁶Tm, ²³⁰U, ²³⁷U, ²⁴⁰U, ⁴⁸V, ¹⁷⁸W, ¹⁸¹W, ¹⁸⁸W, ¹²⁵Xe, ¹²⁷Xe, ¹³³Xe, ¹³³mXe, ¹³⁵Xe, ⁸⁵mY, ⁸⁶Y, ⁹⁰Y, ⁹³Y ¹⁶⁹Yb, ¹⁷⁵Yb, ⁶⁵Zn, ⁷¹mZn, ⁸⁶Zr, ⁹⁵Zr, and/or ⁹⁷Zr.

In particular embodiments, radioactive isotopes can be used as a detectable label or radiolabel.

(xi) Linkers for Recombinantly-Expressed Molecules. As used herein, a linker can include a chemical moiety that serves to connect two other subcomponents of the molecule. Some linkers serve no purpose other than to link components while many linkers serve an additional purpose. Linkers can, for example, link VL and VH of antibody derived binding domains of scFvs and serve as junction amino acids between subcomponent portions of a recombinant receptor.

Linkers can be flexible, rigid, or semi-rigid, depending on the desired function of the linker. Linkers can include junction amino acids. For example, in particular embodiments, linkers provide flexibility and room for conformational movement between different components of recombinant receptor. Commonly used flexible linkers include Gly-Ser linkers. In particular embodiments, the linker sequence includes sets of glycine and serine repeats such as from one to ten repeats of (GlyₓSer_{y})ₙ, wherein x and y are independently an integer from 0 to 10 provided that x and y are not both 0 and wherein n is an integer of 1, 2, 3, 4, 5, 6, 7, 8, 9 or 10). Particular examples include (Gly₄Ser)ₙ (SEQ ID NO: 190), (Gly₃Ser)ₙ(Gly₄Ser)ₙ (SEQ ID NO: 191), (Gly₃Ser)ₙ(Gly₂Ser)ₙ (SEQ ID NO: 192), or (Gly₃Ser)ₙ(Gly₄Ser)₁ (SEQ ID NO: 193). In particular embodiments, the linker is (Gly₄Ser)₄ (SEQ ID NO: 194), (Gly₄Ser)₃ (SEQ ID NO: 195), (Gly₄Ser)₂ (SEQ ID NO: 196), (Gly₄Ser)₁ (SEQ ID NO: 197), (Gly₃Ser)₂ (SEQ ID NO: 198), (Gly₃Ser)₁ (SEQ ID NO: 199), (Gly₂Ser)₂ (SEQ ID NO: 200) or (Gly₂Ser)₁, GGSGGGSGGSG (SEQ ID NO: 201), GGSGGGSGSG (SEQ ID NO: 202), or GGSGGGSG (SEQ ID NO: 203).

In particular embodiments, a linker region is (GGGGS)ₙ (SEQ ID NO: 190) wherein n is an integer including, 1, 2, 3, 4, 5, 6, 7, 8, 9, or more. In particular embodiments, the linker is (EAAAK)ₙ (SEQ ID NO: 204) wherein n is an integer including 1, 2, 3, 4, 5, 6, 7, 8, 9, or more.

In some situations, flexible linkers may be incapable of maintaining a distance or positioning of recombinant receptors needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

Linkers can be susceptible to cleavage (cleavable linker), such as, acid-induced cleavage, photo-induced cleavage, peptidase-induced cleavage, esterase-induced cleavage, and disulfide bond cleavage. Alternatively, linkers can be substantially resistant to cleavage (e.g., stable linker or noncleavable linker). In some aspects, the linker is a procharged linker, a hydrophilic linker, or a dicarboxylic acid-based linker. Junction amino acids can be a linker which can be used to connect sequences when the distance provided by a spacer is not needed and/or wanted. For example, junction amino acids can be short amino acid sequences that can be used to connect co-stimulatory intracellular signaling components. In particular embodiments, junction amino acids are 9 amino acids or less (e.g., 2, 3, 4, 5, 6, 7, 8, or 9 amino acids). In particular embodiments, a glycine-serine doublet can be used as a suitable junction amino acid linker. In particular embodiments, a single amino acid, e.g., an alanine, a glycine, can be used as a suitable junction amino acid.

(xii) Multimerization Domains for Recombinantly-Expressed Molecules. In particular embodiments, a recombinantly-expressed molecule can optionally include a multimerization domain. A “multimerization domain” is a domain that causes two or more proteins (monomers) to interact with each other through covalent and/or non-covalent association(s). Multimerization domains present in proteins can result in protein interactions that form dimers, trimers, tetramers, pentamers, hexamers, heptamers, etc., depending on the number of units/monomers incorporated into the multimer. In particular embodiments, a binding domain molecule includes a multimerization domain.

In particular embodiments, the multimerization domain is based on a basic immunoglobulin structure such as an IgA domain or an IgM domain. Basic immunoglobulin structures in vertebrate systems are described above and are well understood. (See, e.g., Harlow et al., Antibodies: A Laboratory Manual, Cold Spring Harbor Laboratory Press, 2nd ed. 1988).

IgA, similar to IgG, contains three constant domains (CA1-CA3), with a hinge region between the CA1 and CA2 domains. Both forms of IgA, IgA1 and IgA2, have the capacity to form dimers, in which two monomer units, are arranged in an end-to-end configuration stabilized by disulfide bridges and incorporation of a J-chain.

IgAs possess an 18-amino acid extension in the C terminus called the “tail-piece” (tp). The IgA tp is highly conserved among various animal species. The conserved penultimate cysteine residue in the IgA tp has been demonstrated to be involved in multimerization by forming a disulfide bond between heavy chains to permit formation of a multimer. The tp contains an N-linked carbohydrate addition site, the presence of which is required for dimer formation in IgA.

The human IgA1 constant region typically includes the amino acid sequence.

(SEQ ID NO: 205)
ASPTSPKVFPLSLCSTQPDGNVVIACLVQGFFPQEPLSVTWSESGQGVT
ARNFPPSQDASGDLYTTSSQLTLPATQCLAGKSVTCHVKHYTNPSQDVT
VPCPVPSTPPTPSPSTPPTPSPSCCHPRLSLHRPALEDLLLGSEANLTC
TLTGLRDASGVTFTWTPSSGKSAVQGPPERDLCGCYSVSSVLPGCAEPW
NHGKTFTCTAAYPESKTPLTATLSKSGNTFRPEVHLLPPPSEELALNEL
VTLTCLARGFSPKDVLVRWLQGSQELPREKYLTWASRQEPSQGTTTFAV
TSILRVAAEDWKKGDTFSCMVGHEALPLAFTQKTIDRLAGKPTHVNVSV
VMAEVDGTCY.

Referring to this SEQ ID NO: 205, the human CA1 domain extends from amino acid 6 to amino acid 98; the human IgA1 hinge region extends from amino acid 102 to amino acid 124, the human CA2 domain extends from amino acid 125 to amino acid 219, the human CA3 domain extends from amino acid 228 to amino acid 330, and the tp extends from amino acid 331 to amino acid 352.

The human IgA2 constant region typically includes the amino acid sequences

(SEQ ID NO: 206)
ASPTSPKVFPLSLDSTPQDGNVVVACLVQGFFPQEPLSVTWSESGQNVT
ARNFPPSQDASGDLYTTSSQLTLPATQCPDGKSVTCHVKHYTNPSQDVT
VPCPVPPPPPCCHPRLSLHRPALEDLLLGSEANLTCTLTGLRDASGATF
TWTPSSGKSAVQGPPERDLCGCYSVSSVLPGCAQPWNHGETFTCTAAHP
ELKTPLTANITKSGNTFRPEVHLLPPPSEELALNELVTLTCLARGFSPK
DVLVRWLQGSQELPREKYLTWASRQEPSQGTTTFAVTSILRVAAEDWKK
GDTFSCMVGHEALPLAFTQKTIDRLAGKPTHVNVSVVMAEVDGTCY.

Referring to this SEQ ID NO: 206, the human CA1 domain extends from amino acid 6 to amino acid 98, the human IgA2 hinge region extends from amino acid 102 to amino acid 111, the human CA2 domain extends from amino acid 113 to amino acid 206, the human CA3 domain extends from amino acid 215 to amino acid 317, and the tp extends from amino acid 318 to amino acid 340.

As indicated, two IgA binding units can form a complex with two additional polypeptide chains, the J chain (e.g., SEQ ID NO: 207, the mature human J chain) and the secretory component to form a bivalent secretory IgA (sIgA)-derived binding molecule. See, e.g., Braathen, R., el al., J. Biol. Chem. 277:42755-42762 (2002).

Each of the strategies discussed above can be used to create IgA antibody-based dimers. See, e.g., U.S. Pat. Nos. 9,951,134, 10,400,038, and 9,938,347, U.S. Patent Application Publication Nos. US20190100597A1, US20180118814A1, US20180118816A1, US20190185570A1, and US20180265596A1, and PCT Publication Nos. WO 2018/017888, WO 2018/017763, WO 2018/017889, WO 2018/017761, and WO 2019/165340.

IgM domains form hexamers, or in association with a J-chain, pentamers. A “full length IgM antibody heavy chain” is a polypeptide that includes, in N-terminal to C-terminal direction, an antibody heavy chain variable domain (VH), an antibody heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) that can include a tp, as indicated above.

Embodiments with an IgM constant region typically include at least the Cμ4-tailpiece (tp) domains of the IgM constant region but can include heavy chain constant region domains from other antibody isotypes, e.g., IgG, from the same species or from a different species. In particular embodiments, one or more constant region domains can be deleted so long as the IgM antibody is capable of forming hexamers and/or pentamers. Thus, an IgM antibody can be, e.g., a hybrid IgM/IgG antibody or can be a “multimerizing fragment” of an IgM-derived binding molecule.

In particular embodiments, each binding unit of a multimeric binding molecule as provided herein includes two IgM heavy chain constant regions or multimerizing fragments or variants thereof, each including at least an IgM Cμ4 domain and an IgM tp domain. In certain embodiments the IgM heavy chain constant regions can each further include an IgM Cμ3 domain situated N-terminal to the IgM Cμ4 and IgM tp domains.

The human IgM constant region typically includes the amino acid sequence

GSASAPTLFPLVSCENSPSDTSSVAVGCLAQDFLPDSITFSWKYKNNSDISSTRGFPSVLRGGK
YAATSQVLLPSKDVMQGTDEHVVCKVQHPNGNKEKNVPLPVIAELPPKVSVFVPPRDGFFGNP
RKSKLICQATGFSPRQIQVSWLREGKQVGSGVTTDQVQAEAKESGPTTYKVTSTLTIKESDWL
SQSMFTCRVDHRGLTFQQNASSMCVPDQDTAIRVFAIPPSFASIFLTKSTKLTCLVTDLTTYDSV
TISWTRQNGEAVKTHTNISESHPNATFSAVGEASICEDDWNSGERFTCTVTHTDLPSPLKQTIS
RPKGVALHRPDVYLLPPAREQLNLRESATITCLVTGFSPADVFVQWMQRGQPLSPEKYVTSAP
MPEPQAPGRYFAHSILTVSEEEWNTGETYTCVVAHEALPNRVTERTVDKSTGKPTLYNVSLVM
SDTAGTCY (SEQ ID NO: 208; identical to, e.g., GenBank Accession
Nos. pir||S37768, CAA47708.1, and CAA47714.1).

Referring to this SEQ ID NO: 208, the human Cμ1 region ranges from amino acid 5 to amino acid 102; the human Cμ2 region ranges from amino acid 114 to amino acid 205, the human Cμ3 region ranges from amino acid 224 to amino acid 319, the Cμ4 region ranges from amino acid 329 to amino acid 430, and the tp ranges from amino acid 431 to amino acid 453.

In particular embodiments, an IgM heavy chain constant region includes the sequence:

(SEQ ID NO: 209; (UniProt ID P01871)
GSASAPTLFPLVSCENSPSDTSSVAVGCLAQDFLPDSITFSWKYKNNSD
ISSTRGFPSVLRGGKYAATSQVLLPSKDVMQGTDEHVVCKVQHPNGNKE
KNVPLPVIAELPPKVSVFVPPRDGFFGNPRKSKLICQATGFSPRQIQVS
WLREGKQVGSGVTTDQVQAEAKESGPTTYKVTSTLTIKESDWLGQSMFT
CRVDHRGLTFQQNASSMCVPDQDTAIRVFAIPPSFASIFLTKSTKLTCL
VTDLTTYDSVTISWTRQNGEAVKTHTNISESHPNATFSAVGEASICEDD
WNSGERFTCTVTHTDLPSPLKQTISRPKGVALHRPDVYLLPPAREQLNL
RESATITCLVTGFSPADVFVQWMQRGQPLSPEKYVTSAPMPEPQAPGRY
FAHSILTVSEEEWNTGETYTCVVAHEALPNRVTERTVDKSTGKPTLYNV
SLVMSDTAGTCY.

allele IGHM04). In certain aspects, a variant human IgM constant region includes an amino acid sequence with 90% sequence identity to SEQ ID NOs: 208 or 209. As indicated, both IgA and IgM binding units can form a complex with a J-chain. The mature human J-chain includes the amino acid sequence:

(SEQ ID NO: 207)
QEDERIVLVDNKCKCARITSRIIRSSEDPNEDIVERNIRIIVPLNNREN
ISDPTSPLRTRFVYHLSDLCKKCDPTEVELDNQIVTATQSNICDEDSAT
ETCYTYDRNKCYTAVVPLVYGGETKMVETALTPDACYPD.

The term “J-chain” as used herein refers to the J-chain of native sequence IgA or IgM antibodies of any animal species. When specified, it can also refer to any functional fragment thereof, derivative thereof, and/or variant thereof, including a mature human J-chain amino acid sequence provided herein as SEQ ID NO: 207. A functional fragment, derivative, and/or variant of a J-chain has at least 90% sequence identity to the reference J-chain and retains the multimerizing function of the reference J-chain.

In particular embodiments, the multimerization domain is a dimerization domain that allows binding of two complementary monomers to form a dimer. In particular embodiments, a dimerization and docking domain (DDD) can be derived from the cAMP-dependent protein kinase (PKA) regulatory subunits and can be paired with an anchoring domain (AD). The AD can be derived from a specific region found in various A-kinase anchoring proteins (AKAPs) that mediates association with the R subunits of PKA. Additional DDDs and ADs include: the 4-helix bundle type DDD (Newlon, et al., EMBO J. 2001; 20: 1651-1662; Newlon, et al., Nature Struct Biol. 1999; 3: 222-227) domains obtained from p53, DCoH (pterin 4 a carbinolamine dehydratase/dimerization cofactor of hepatocyte nuclear factor 1 α (TCF1)) and HNF-1 (hepatocyte nuclear factor 1) (Rose, et al., Nature Struct Biol. 2000; 7: 744-748). Other AD sequences of potential use may be found in US 2003/0232420A1.

In particular embodiments, complementary binding domains can dimerize. In particular embodiments, the binding domain is a transmembrane polypeptide derived from a FcϵRI chain. In particular embodiments, a recombinant receptor can include a part of a FcϵRI α chain and another recombinant receptor can include a part of an FcϵRI β chain such that said FcϵRI chains spontaneously dimerize together to form a dimeric recombinant receptor. In particular embodiments, recombinant receptor can include a part of a FcϵRI α chain and a part of a FcϵRI γ chain such that said FcϵRI chains spontaneously trimerize together to form a trimeric recombinant receptor, and in another embodiment the multi-chain recombinant receptor can include a part of FcϵRI α chain, a part of FcϵRI β chain and a part of FcϵRI γ chain such that said FcϵRI chains spontaneously tetramerize together to form a tetrameric recombinant receptor.

Leucine zippers are described in U.S. Pat. No. 5,932,448; SH2 and SH3 are described in Vidal et al., Biochemistry, 43:7336-44, 2004); PTB is described in Zhou et al., Nature, 378:584-592, 1995); WW is described in Sudol Prog Biochys MoL Bio, 65:113-132, 1996; PDZ is described in Kim et al., Nature, 378: 85-88, 1995 and Komau et al., Science, 269:1737-1740, 1995; and WD40 is described in Hu et al., J Biol Chem., 273:33489-33494, 1998.

Additional multimerization domains and systems are described in, for example, Hodneland, et al., Proc Natl Acd Sci USA. 2002; 99: 5048-5052; Arakawa et al., J Biol. Chem., 269:27833-27839, 1994; Radziejewski et al., Biochem, 32: 1350, 1993; WO2012001647A2; U.S. Pat. No. 5,821,333; GenBank Accession no. AAF73912.1 (Nishi et al., Mol Cell Biol, 25: 2607-2621, 2005), the SH3 domain of I1l from GenBank Accession no. AAD22543.1 (Kristensen el al., EMBO J., 25: 785-797, 2006), the PTB domain of human DOK-7 from GenBank Accession no. NP_005535.1 (Wagner et al., Cold Spring Harb Perspect Biol. 5: a008987, 2013), the PDZ-like domain of SATB1 from UniProt Accession No. Q01826 (Galande et al., Mol Cell Biol. Aug; 21: 5591-5604, 2001), the WD40 repeats of APAF from UniProt Accession No. 014727 (Jorgensen et al., 2009. PLOS One. 4(12):e8463), the PAS motif of the dioxin receptor from UniProt Accession No. 16L9E7 (Pongratz et al., Mol Cell Biol, 18:4079-4088, 1998) and the EF hand motif of parvalbumin from UniProt Accession No. P20472 (Jamalian et al., Int J Proteomics, 2014: 153712, 2014). C4b, dextrameric, and ferritin-based multimerization can be used.

In particular embodiments, complementary binding domains can be induced using a third molecule or chemical inducer. This method of dimerization requires that one recombinant receptor include a chemical inducer of dimerization binding domain 1 (CBD1) and the second recombinant receptor include the second chemical inducer of dimerization binding domain (CBD2), wherein CBD1 and CBD2 are capable of simultaneously binding to a chemical inducer of dimerization (CID). CBD1 may include a rapamycin binding domain of FK-binding protein 12 (FKBP12) and CBD2 may include a FKBP12-Rapamycin Binding (FRB) domain of mTOR.

(xiii) Antibody Compositions for Administration. In particular embodiments, antibody-based therapeutics described herein (e.g., T antigen of the Merkel cell polyomavirus antibodies, multi-specific binding domain molecules, antibody conjugates) in any exemplary format can be formulated alone or in combination into compositions for administration to subjects. Antibody-based therapeutics are referred to herein as “active ingredients”.

Salts and/or pro-drugs of the active ingredients can also be used.

A pharmaceutically acceptable salt includes any salt that retains the activity of the active ingredient and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage or by hydrolysis of a biologically labile group.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA (ethylene-diamine-tetra-acetic acid).

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the antibodies or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The compositions disclosed herein can be formulated for administration by, for example, injection, inhalation, infusion, perfusion, lavage, or ingestion. The formulations and/or compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral, sublingual, and/or subcutaneous administration.

For injection, compositions can be formulated as aqueous solutions, such as in buffers including Hanks' solution, Ringer's solution, or physiological saline. The aqueous solutions can include formulatory agents such as suspending, stabilizing, and/or dispersing agents.

Alternatively, the composition can be in lyophilized and/or powder form for constitution with a suitable vehicle, e.g., sterile pyrogen-free water, before use.

For oral administration, the compositions can be formulated as tablets, pills, dragées, capsules, liquids, gels, syrups, slurries, suspensions and the like. For oral solid compositions such as powders, capsules and tablets, suitable excipients include binders (gum tragacanth, acacia, cornstarch, gelatin), fillers such as sugars, e.g., lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of the composition and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Additionally, compositions can be formulated as sustained-release systems utilizing semipermeable matrices of solid polymers including at least one type of antibody. Various sustained-release materials have been established and are well known by those of ordinary skill in the art. Sustained-release systems may, depending on their chemical nature, release one or more antibodies following administration for a few weeks up to over 100 days. Depot preparations can be administered by injection; parenteral injection; instillation; or implantation into soft tissues, a body cavity, or occasionally into a blood vessel with injection through fine needles.

Depot compositions can include a variety of bioerodible polymers including poly(lactide), poly(glycolide), poly(caprolactone) and poly(lactide)-co(glycolide) (PLG) of desirable lactide:glycolide ratios, average molecular weights, polydispersities, and terminal group chemistries. Blending different polymer types in different ratios using various grades can result in characteristics that borrow from each of the contributing polymers.

The use of different solvents (for example, dichloromethane, chloroform, ethyl acetate, triacetin, N-methyl pyrrolidone, tetrahydrofuran, phenol, or combinations thereof) can alter microparticle size and structure in order to modulate release characteristics. Other useful solvents include water, ethanol, dimethyl sulfoxide (DMSO), N-methyl-2-pyrrolidone (NMP), acetone, methanol, isopropyl alcohol (IPA), ethyl benzoate, and benzyl benzoate.

Exemplary release modifiers can include surfactants, detergents, internal phase viscosity enhancers, complexing agents, surface active molecules, co-solvents, chelators, stabilizers, derivatives of cellulose, (hydroxypropyl)methyl cellulose (HPMC), HPMC acetate, cellulose acetate, pluronics (e.g., F68/F127), polysorbates, Span® (Croda Americas, Wilmington, Delaware), poly(vinyl alcohol) (PVA), Brij® (Croda Americas, Wilmington, Delaware), sucrose acetate isobutyrate (SAIB), salts, and buffers.

Excipients that partition into the external phase boundary of nanoparticles such as surfactants including polysorbates, dioctylsulfosuccinates, poloxamers, PVA, can also alter properties including particle stability and erosion rates, hydration and channel structure, interfacial transport, and kinetics in a favorable manner.

Additional processing of the disclosed sustained release depot compositions can utilize stabilizing excipients including mannitol, sucrose, trehalose, and glycine with other components such as polysorbates, PVAs, and dioctylsulfosuccinates in buffers such as Tris, citrate, or histidine. A freeze-dry cycle can also be used to produce very low moisture powders that reconstitute to similar size and performance characteristics of the original suspension.

In particular embodiments, the compositions include active ingredients of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

As described earlier for cell-based formulations, any composition disclosed herein can advantageously include any other pharmaceutically acceptable carriers which include those that do not produce significantly adverse, allergic, or other untoward reactions that outweigh the benefit of administration. Moreover, compositions can be prepared to meet sterility, pyrogenicity, general safety, and purity standards as required by U.S. FDA Office of Biological Standards and/or other relevant foreign regulatory agencies.

(xiv) Methods of Use—Treatment. In particular embodiments, formulations and/or compositions disclosed herein can be used for treating subjects (e.g., cell-based immunotherapies, antibody-based therapies). Methods disclosed herein include treating subjects (humans, non-human primates, veterinary animals (dogs, cats, reptiles, birds, etc) livestock (horses, cattle, goats, pigs, chickens, etc) and research animals (monkeys, rats, mice, fish, etc)) with formulations and/or compositions disclosed herein. Treating subjects includes delivering therapeutically effective amounts. Therapeutically effective amounts include those that provide effective amounts, prophylactic treatments and/or therapeutic treatments.

An “effective amount” is the amount of a formulation necessary to result in a desired physiological change in the subject. For example, an effective amount can provide an immunogenic anti-cancer effect. Effective amounts are often administered for research purposes. Effective amounts disclosed herein can cause a statistically significant effect in an animal model or in vitro assay relevant to the assessment of a cancer's development or progression. An immunogenic formulation can be provided in an effective amount, wherein the effective amount stimulates an immune response.

A “prophylactic treatment” includes a treatment administered to a subject who does not display signs or symptoms of a cancer or displays only early signs or symptoms of a cancer such that treatment is administered for the purpose of diminishing or decreasing the risk of developing the cancer further. Thus, a prophylactic treatment functions as a preventative treatment against a T antigen of the Merkel cell polyomavirus-expressing cancer. In particular embodiments, prophylactic treatments reduce, delay, or prevent metastasis from a primary cancer tumor site from occurring.

A “therapeutic treatment” includes a treatment administered to a subject who displays symptoms or signs of a cancer and is administered to the subject for the purpose of diminishing or eliminating those signs or symptoms of the cancer. The therapeutic treatment can reduce, control, or eliminate the presence or activity of the cancer and/or reduce control or eliminate side effects of the cancer.

Function as an effective amount, prophylactic treatment or therapeutic treatment are not mutually exclusive, and in particular embodiments, administered dosages may accomplish more than one treatment type.

In particular embodiments, therapeutically effective amounts provide anti-cancer effects. Anti-cancer effects include a decrease in the number of cancer cells, decrease in the number of metastases, a decrease in tumor volume, an increase in life expectancy, induced chemo- or radiosensitivity in cancer cells, inhibited angiogenesis near cancer cells, inhibited cancer cell proliferation, inhibited tumor growth, prevented or reduced metastases, prolonged subject life, reduced cancer-associated pain, and/or reduced relapse or re-occurrence of cancer following treatment.

A “tumor” is a swelling or lesion formed by an abnormal growth of cells (called neoplastic cells or tumor cells). A “tumor cell” is an abnormal cell that grows by a rapid, uncontrolled cellular proliferation and continues to grow after the stimuli that initiated the new growth cease. Tumors show partial or complete lack of structural organization and functional coordination with the normal tissue, and usually form a distinct mass of tissue, which may be benign, pre-malignant or malignant.

In particular embodiments, therapeutically effective amounts induce an immune response. The immune response can be against the T antigen of the Merkel cell polyomavirus-expressing cell.

“T antigen of the Merkel cell polyomavirus-positive cell” refers to a cell that presents a T antigen peptide or fragment thereof in complex with a major histocompatibility complex (MHC) (e.g., human leukocyte antigen) molecule, wherein the MHC molecule is on the cell surface. In particular embodiments, the T antigen of the Merkel cell polyomavirus-positive cell is a cancer cell. In some embodiments, expression of a T antigen peptide or fragment thereof on an MHC molecule is determined, for example, using antibodies to the T antigen of the Merkel cell polyomavirus in a method such as immunohistochemistry, FACS, etc. Alternatively, T antigen peptide mRNA expression is considered to correlate to expression of the T antigen of the Merkel cell polyomavirus and can be determined by, for example, in situ hybridization and/or RT-PCR (including quantitative RT-PCR).

In particular embodiments, a T antigen-related disorder includes the aggressive human skin cancer called Merkel cell carcinoma. The Merkel cell polyomavirus (MCPyV) large T antigen is frequently found truncated in Merkel cell carcinomas. The majority of Merkel cell carcinomas are associated with infection by MCPyV.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of cancer, stage of, previous or concurrent therapeutic interventions, idiopathy of the subject and route of administration.

Therapeutically effective amounts of formulations can include 10⁴ to 10⁹ cells/kg body weight, or 10³ to 10¹¹ cells/kg body weight. Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

Therapeutically effective amounts of compositions can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular embodiments, the treatment protocol may be dictated by a clinical trial protocol or an FDA-approved treatment protocol.

Therapeutically effective amounts can be administered by, e.g., injection, infusion, perfusion, or lavage. Routes of administration can include bolus intravenous, intradermal, intraarterial, intraparenteral, intranodal, intralymphatic, intraperitoneal, intralesional, intraprostatic, intravaginal, intrarectal, topical, intrathecal, intratumoral, intramuscular, intravesical, and/or subcutaneous administration. In particular embodiments, cell formulations and/or modifying formulations are administered by intravesically.

In particular embodiments, formulations and/or compositions are administered to a patient in conjunction with (e.g., before, simultaneously or following) any number of relevant treatment modalities. In particular embodiments, cells may be used in combination with chemotherapy, radiation, immunosuppressive agents, anti-inflammatory agents, cytokines, immunoablative agents, or other antibody therapies.

Methods of use described herein also assess for the presence of T-Ag-specific B cells in tumor-infiltrated lymph nodes. When T-Ag-specific germinal center B cells are detected in lymph node samples, the patient usually remains MCC-free for a significant period of time (years). If T-Ag-specific germinal B cells are not detected in lymph node samples, then the patient should be administered an additional therapy for MCC because it is likely to progress. These embodiments can include obtaining a sample derived from a tumor-infiltrated lymph node derived from a patient and assessing the sample for the presence of T-Ag-specific germinal center B cells. If T-Ag-specific germinal center B cells are detected or are detected above a relevant threshold, a patient can be scheduled for monitoring. If T-Ag-specific germinal center B cells are not detected or are detected below a relevant threshold, a patient can be scheduled for treatment. Methods to detect and quantify the presence of T-Ag-specific germinal center B cells are known.

(xv) Methods of Use—In Vivo Detection. Antibodies described herein can also be used for in vivo detection or imaging of T antigen of the Merkel cell polyomavirus-expressing cells (e.g., Merkel cell carcinoma cells). In particular embodiments, detection is for research, diagnostic, and/or prognostic uses. In particular embodiments, methods of in vivo detection include administering an effective amount of a composition disclosed herein to a subject in need thereof.

In particular embodiments, the composition includes an antibody-based therapeutic such as antibodies or fragments thereof, multi-specific binding domain molecules, and/or antibody conjugates. In particular embodiments, a composition is used for in vivo detection of a T antigen (e.g., a Merkel cell carcinoma cell expressing the T antigen of the Merkel cell polyomavirus).

In particular embodiments, an antibody-based therapeutic (e.g., antibody conjugate) includes a binding domain as disclosed herein linked to a detectable label as described elsewhere herein. The detectable label can be any label capable of producing, either directly or indirectly, a detectable signal from within the body. In other embodiments, antibody-based therapeutics (e.g., antibodies or fragments thereof, multi-specific binding domain molecules, and/or antibody conjugates) include a domain that binds to a molecule including the detectable label.

In particular embodiments, in vivo detection is useful in diagnosis and/or prognosis of a subject's condition. The term “diagnosis”, as used herein, refers to evaluation of the presence or properties of pathological states or lack thereof. With respect to objects of the present disclosure, in particular embodiments, the diagnosis is to determine the presence of a T antigen-expressing cell, such as a Merkel cell carcinoma cell.

As used herein, prognosis of the subject's condition can refer to evaluating indicia of a cancer disease at a given time point and comparing it to the same indicia of the cancer disease taken at an earlier time point, wherein the comparison is indicative of a progression of the cancer in the subject. In some embodiments, progression of the cancer includes metastasis of the cancer in the subject.

In general, a method of in vivo detection includes administering a composition as disclosed herein to a subject. The composition includes an antibody-based therapeutic (e.g., antibody, antibody fragment, multi-specific binding domain molecule, and/or antibody conjugate), wherein the antibody-based therapeutic can be conjugated to a detectable label (e.g., antibody conjugate) or the detectable label can otherwise become associated with the antibody during the methods. Once the composition has had sufficient time for binding within the subject, the detectable label is detected and/or quantified. In particular embodiments, the detectable label can be compared to a reference level. The term “time sufficient for binding” refers to a temporal duration that permits binding of the labeled agent to a target molecule.

In particular embodiments, a method of in vivo detection includes administering to a subject, an antibody-based therapeutic, wherein the antibody-based therapeutic includes a detectable label; and detecting and/or quantifying the detectable label within the subject.

In particular embodiments, a method of in vivo detection includes administering to a subject, an antibody-based therapeutic, wherein the antibody-based therapeutic includes a domain that binds a molecule including a detectable label; administering the molecule including the detectable label; and detecting and/or quantifying the detectable label within the subject.

Detection and imaging of the antibody is tunable, such that imaging can be performed in under 1, 2, 4, 6, 12, or 18, 24, 36, or 48 hours, or any amount below, above, or between this amount. It has been demonstrated that PEGs/larger molecules increase serum half-life by 2, 3, 4, 5, 6, 7, 8, 9, 10, 15, 20, 25, 30, 35, 40, 45, 50, 55, 60, 65, 70, 75, 80, 85, 90, 95, or 100%, or 2, 3, 4, 5, 6, 7, 8, 9, or 10 or more times compared to a smaller molecule. This allows for imaging at different time points.

In particular embodiments, a composition of the presently disclosed subject matter includes a label that can be detected in vivo. The term “in vivo” as used herein to describe imaging or detection methods, refers to generally non-invasive methods such as fluorescence, scintigraphic methods, magnetic resonance imaging, autoradiographic detection, or radioimmunoguided systems, each described briefly herein below. The term “non-invasive methods” includes methods employing administration of a contrast agent to facilitate in vivo imaging. In vivo imaging can be useful in the staging and treatment of malignancies.

Scintigraphic imaging methods include SPECT (Single Photon Emission Computed Tomography). PET (Positron Emission Tomography), gamma camera imaging, and rectilinear scanning. A gamma camera and a rectilinear scanner each represent instruments that detect radioactivity in a single plane. Most SPECT systems are based on the use of one or more gamma cameras that are rotated about the subject of analysis, and thus integrate radioactivity in more than one dimension. PET systems include an array of detectors in a ring that also detect radioactivity in multiple dimensions.

Imaging instruments suitable for practicing the detection and/or imaging methods of the presently disclosed subject matter, and instruction for using the same, are readily available from commercial sources. For example, a SPECT scanner can be used with a CT scanner, with coregistration of images. As in PET/CT, this allows location of tumors or tissues which may be seen on SPECT scintigraphy but are difficult to precisely locate with regard to other anatomical structures. Both PET and SPECT systems are offered by ADAC of Milpitas, Calif., United States of America, and Siemens of Hoffman Estates, Ill., United States of America. Related devices for scintigraphic imaging can also be used, such as a radio-imaging device that includes a plurality of sensors with collimating structures having a common source focus.

When scintigraphic imaging is employed, the detectable label can include a radiolabel as described elsewhere herein. When the labeling moiety is a radionuclide, stabilizers to prevent or minimize radiolytic damage, such as ascorbic acid, gentisic acid, or other appropriate antioxidants, can be added to the composition including the labeled targeting molecule.

Magnetic resonance image-based techniques create images based on the relative relaxation rates of water protons in unique chemical environments. As used herein, the term “magnetic resonance imaging” refers to magnetic source techniques including conventional magnetic resonance imaging, magnetization transfer imaging (MTI), proton magnetic resonance spectroscopy (MRS), diffusion-weighted imaging (DWI) and functional MR imaging.

Those skilled in the art of diagnostic labeling recognize that metal ions can be bound by chelating moieties, which in turn can be conjugated to a therapeutic agent in accordance with the methods of the presently disclosed subject matter. For example, gadolinium ions are chelated by diethylenetriaminepentaacetic acid (DTPA). Lanthanide ions are chelated by tetraazacyclododocane compounds. See U.S. Pat. Nos. 5,738,837 and 5,707,605. Alternatively, a contrast agent can be carried in a liposome.

Images derived used a magnetic source can be acquired using, for example, a superconducting quantum interference device magnetometer (SQUID, available with instruction from Quantum Design of San Diego, Calif., United States of America; see also U.S. Pat. No. 5,738,837).

In the case of a radioisotope (also referred to herein as radiolabel) detection can be accomplished by conventional autoradiography or by using a phosphorimager as is known to one of skill in the art. In particular embodiments, an autoradiographic method employs photostimulable luminescence imaging plates (Fuji Medical Systems of Stamford, Conn., United States of America). Briefly, photostimulable luminescence is the quantity of light emitted from irradiated phosphorous plates following stimulation with a laser during scanning. The luminescent response of the plates is linearly proportional to the activity.

Another application of the antibodies disclosed herein is in the radioimmunoguided surgery (RIGS) system. This technique involves the intravenous administration of a radiolabeled antibody prior to surgery. After allowing for tumor uptake and blood clearance of radioactivity, the patient is taken to the operating room where surgical exploration is affected with the aid of a hand-held gamma activity probe, e.g., Neoprobe®1000 (Neoprobe Corporation, Dublin, Ohio). This helps the surgeon identify the tumor metastases and improve the complications of excision. The RIGS system is advantageous because it allows for the detection of tumors not otherwise detectable by visual inspection and/or palpation. See, O'Dwyer et al, Arch. Surg., 121:1 391-1394 (1986). This technique is described in detail in Hinkle et al, Antibody, Immunoconjugates and Radiopharmacouticals, 4:(3)339-358 (1991).

Fluorescence imaging can be performed in vivo to detect a detectable label within a subject. For in vivo detection of a fluorescent label, an image is created using emission and absorbance spectra that are appropriate for the particular label used. The image can be visualized, for example, by diffuse optical spectroscopy. Additional methods and imaging systems are described in U.S. Pat. Nos. 5,865,754; 6,083,486; and 6,246,901, among other places.

(xvi) Ex Vivo Detection Assays and Methods. A detection assay can be prepared using capture molecules including the antibody-based therapeutics (e.g., antibodies, antibody fragments, multi-specific binding domain molecules, and/or antibody conjugates) described herein. In particular embodiments, the detection assay includes immunobeads, immunoassays, and lateral flow assays.

In particular embodiments, capture molecules can either be free or immobilized to a solid support. In particular embodiments, a solid support includes slides, wells, chips, pins, filters, beads, or membrane. In particular embodiments, the solid substrate can include or be coated with any material known in the art including polystyrene, glass, gold, nylon, nitrocellulose, polyvinylidene fluoride (PVDF), paper, or latex.

In particular embodiments, capture molecules are immobilized to the solid support using known techniques. In particular embodiments, the capture molecules are immobilized on the solid support via covalent or noncovalent interactions or by passive binding. In particular embodiments, covalent interactions include crosslinking of solvent exposed amino acids such as in octyne-azide chemistry, alkyne-azide chemistry, BCN-azide chemistry, trans-cyclotene-tetrazine chemistry, copper-catalyzed azide-alkyne chemistry, carbodiimide crosslinker chemistry, or maleimide chemistry. In particular embodiments, noncovalent interactions include ionic bonds, hydrophobic interactions, hydrogen bonds, Van der Waals forces, and dipole-dipole bonds.

In certain instances, the capture molecules include capture tags which interact with capture agents bound to the solid support. The detection assays described herein can include a plurality of different capture molecules and/or capture molecule concentrations that are coupled to the surface of a solid support in different known/addressable locations.

The term “capture molecule” is intended to include an immobilized antibody which is specific for (i.e., binds, is bound by, or forms a complex with) one or more analytes of interest in a sample such as a cellular extract of circulating cells of a solid tumor. In particular embodiments, the capture molecule is restrained on a solid support in an array.

In general, the detection assay is based on the detection of the antibody binding to its antigen which is well known in the art and may be achieved through the application of numerous approaches. These methods are based upon the detection of a label or marker, such as any of those fluorescent, chemiluminescent, spectral colorimetric, enzymatic, affinity tag, or radioactive labels described elsewhere herein. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection (or detection molecule) may itself be linked to a detectable label, wherein one would then simply detect this label, thereby allowing the amount of the primary immune complexes to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected. In particular embodiments, the detection assay includes a detection molecule. The term “detection molecule” as used herein includes an antibody, antibody fragment, or antibody conjugate as described herein that includes a detectable label. The term also encompasses an antibody or antibody fragment as described herein, wherein the antibody or antibody fragment can be bound by another species that includes a detectable label.

In particular embodiments, detection assays are used for ex vivo or in vitro detection of the T antigen of Merkel cell polyomavirus. In particular embodiments, detection is for research, diagnostic, and/or prognostic uses. In particular embodiments, methods of detection include contacting an effective amount of the composition disclosed herein with a biological sample. The antibody-based therapeutics (e.g., antibodies, antibody fragments, multi-specific binding domain molecules, and antibody conjugates) can be, directly or indirectly, associated with or linked to a detectable label, and the composition can be suitable for detection of the T antigen of the Merkel cell polyomavirus or a T antigen-expressing cell (e.g., Merkel cell carcinoma cell).

In general, a method of ex vivo detection includes contacting a biological sample with a composition as disclosed herein. The composition includes an antibody-based therapeutic (e.g., antibody, antibody fragment, multi-specific binding domain molecule, and/or antibody conjugate), wherein the antibody-based therapeutic can be conjugated to a detectable label (e.g., antibody conjugate) or the detectable label can otherwise become associated with the antibody during the methods. Once the biological sample and composition have had sufficient time for binding, the detectable label is detected and/or quantified. In particular embodiments, the detectable label can be compared to a reference level.

In particular embodiments, a method of ex vivo detection includes contacting an antibody-based therapeutic and a biological sample, wherein the antibody-based therapeutic includes a detectable label; and detecting and/or quantifying the detectable label within the biological sample.

In particular embodiments, a method of ex vivo detection includes contacting an antibody-based therapeutic and a biological sample, wherein the antibody-based therapeutic includes a domain that binds a molecule including a detectable label; administering the molecule including the detectable label to the biological sample and composition; and detecting and/or quantifying the detectable label within the subject.

In particular embodiments, the contacting includes applying the antibody-based therapeutic to the biological sample. In particular embodiments, the contacting includes applying the biological sample to a detection assay including the antibody-based therapeutic.

Methods may be used to monitor disease progression, for example, using biopsy samples at different times. In such aspects, instead of comparing the expression of the T antigen of the Merkel cell polyomavirus against a control sample from, e.g., a different tissue source or subject known not to have enhanced T antigenexpression, the expression of the T antigen is compared against a biological sample obtained from the same tissue or the same subject at an earlier time point, for example, from days, weeks or months earlier.

Any suitable biological sample may be used; the nature of the disease or condition may determine the nature of the sample which is to be used in the methods. The sample may be, for example, a sample from a tissue biopsy, tumor tissue biopsy, bone marrow biopsy, or circulating cells in, e.g., blood. Alternatively, e.g., where, for example, the methods are being used to diagnose or monitor a gastrointestinal tumor, tumor cells may be isolated from feces (stool) samples. Other sources of biological sample may include plasma, serum, cerebrospinal fluid, urine, interstitial fluid, ascites fluid or the like.

For example, solid tumor samples may be collected in complete tissue culture medium with antibiotics. Cells may be manually teased from the tumor specimen or, where necessary, are enzymatically disaggregated by incubation with collagenase/DNAse and suspended in appropriate media containing, for example, human or animal sera.

In other aspects, biopsy samples may be isolated and frozen or fixed in fixatives such as formalin. The samples may then be tested for expression levels of genes at a later stage.

The antibodies of the presently disclosed subject matter can be employed in various ex vivo or in vitro assay methods, such as ELISA, Immunohistochemistry, Electron Microscopy, Latex agglutination, lateral flow immunoassays, Immuno Blotting, and Dip Stick Immuno testing, competitive binding assays, direct and indirect sandwich assays, immunoprecipitation assays (see e.g., Zola, 1987; Harlow & Lane, 1988), and as affinity purification agents.

In some embodiments, the detection assay can use affinity tags. If an affinity tag has been used, a protein or compound that binds the affinity tag can be used to detect the affinity tag. Representative affinity tags are described elsewhere herein. In particular embodiments, a protein or compound that binds the affinity tag can be conjugated to a detectable label. In particular embodiments, a protein or compound that binds the affinity tag is conjugated to an enzymatic label. In particular embodiments, a protein or compound that binds the affinity tag is conjugated to an enzymatic label and is detected by the production of a colorimetric or luminescent product that is measurable using a spectrophotometer or luminometer, respectively.

In particular embodiments, the detection assay includes immunobeads. In particular embodiments, the immunobeads include antibody conjugated glass beads or antibody conjugated magnetic beads. In particular embodiments, beads are conjugated with antibodies or fragments thereof using covalent crosslinking methods. In particular embodiments, immunobeads or other immune-conjugated materials can be added to a column. The immunobeads can be added to biological sample, wherein the immunobead will bind the target antigen. In particular embodiments, the immunobeads can be separated from the biological sample and binding can be detected.

In an immunoassay, the antibodies or fragments thereof of the disclosure are immobilized onto a selected surface exhibiting protein affinity, such as a well in a polystyrene microtiter plate. Then, a test composition is added to the wells. After binding and washing to remove non-specifically bound immune complexes, the bound antigen may be detected. Detection may be achieved by the addition of a detection molecule.

In coating a plate for the immunoassay with the capture molecule, one will generally incubate the wells of the plate with a solution of the capture molecule, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface. In particular embodiments, the immunoassay includes an enzyme-linked immunosorbent assay (ELISA).

In particular embodiments, the detection assay includes a lateral flow assay. In particular embodiments, the lateral flow assay is based on a series of capillary beds, such as pieces of porous paper or sintered polymer. Each of these elements has the capacity to transport fluid spontaneously. The first element (the sample pad) acts as a sponge and holds an excess of sample fluid. Once soaked, the fluid migrates to the series of capillary beds including a matrix to facilitate a chemical reaction between the target molecule (e.g., an antigen) and its capture molecule that has been immobilized on the capture bed. In particular embodiments, the capture molecule is immobilized at specific locations within the series of capture beds. In particular embodiments, the analyte binds to the capture molecule while migrating further through the capillary bed. Lateral Flow Tests can operate as either competitive or sandwich assays. Lateral flow assays are disclosed in U.S. Pat. No. 6,485,982.

Immunohistochemistry (IHC) can use the antibody disclosed herein to detect T antigen of the Merkel cell polyomavirus-expressing cells. IHC detects target molecules through antigen-antibody complexes in a pathological specimen using enzyme-linked antigens or antibodies. The presence of the target molecule can then be detected via an enzyme immunoassay.

A multitude of benefits are realized with IHC versus traditional immunofluorescence. For example, unlike immunofluorescence, IHC can be used with commonly used formalin-fixed paraffin-embedded tissue specimens. In particular embodiments, IHC is used on fresh-frozen samples or formalin-fixed paraffin-embedded samples. Pathological specimens, including histological tissue sections and/or other biological preparations such as tissue culture cells, are commonly used in diagnostic pathology and can be easily screened via IHC. Further, IHC staining is permanent and preserves cell morphology. A comparison of the cell morphology and antigen proliferation on two different slides can be useful in monitoring the progression of a disease.

Once an antibody detectable label conjugate has been attached, either directly or indirectly, to the specimen, a substrate, specific for the enzyme, is added to the specimen. When the substrate is added, the enzyme label converts the substrate causing a color change that can be seen with light microscopy. The presence of a color change indicates the presence of the target molecule and allows an observer to determine, assess, and diagnose the disease level and severity.

A protein immunoblot, or a western blot, is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Fluorescence imaging can be used to detect a fluorescent label. Similar to the in vivo fluorescent imaging described earlier, fluorescent imaging can be performed ex vivo.

(xvii) Reference Levels and Experimental Controls. Obtained values for parameters associated with a therapy described herein can be compared to a reference level derived from a control population, and this comparison can indicate whether a therapy described herein is effective for a subject in need thereof. Reference levels can be obtained from one or more relevant datasets from a control population. A “dataset” as used herein is a set of numerical values resulting from evaluation of a sample (or population of samples) under a desired condition. The values of the dataset can be obtained, for example, by experimentally obtaining measures from a sample and constructing a dataset from these measurements. As is understood by one of ordinary skill in the art, the reference level can be based on e.g., any mathematical or statistical formula useful and known in the art for arriving at a meaningful aggregate reference level from a collection of individual data points; e.g., mean, median, median of the mean, etc. Alternatively, a reference level or dataset to create a reference level can be obtained from a service provider such as a laboratory, or from a database or a server on which the dataset has been stored.

A reference level from a dataset can be derived from previous measures derived from a control population. A “control population” is any grouping of subjects or samples of like specified characteristics. The grouping could be according to, for example, clinical parameters, clinical assessments, therapeutic regimens, disease status, severity of condition, etc. In particular embodiments, the grouping is based on age range (e.g., 60-65 years) and cancer status. In particular embodiments, a normal control population includes individuals that are age-matched to a test subject and do not have cancer. In particular embodiments, age-matched includes, e.g., 0-10 years old; 30-40 years old, 60-65 years old, 70-85 years old, etc., as is clinically relevant under the circumstances. In particular embodiments, a control population can include those that have a Merkel cell carcinoma and have not been administered a therapeutically effective amount of formulations and/or compositions.

In particular embodiments, the relevant reference level for values of a particular parameter associated with a therapy described herein is obtained based on the value of a particular corresponding parameter associated with a therapy in a control population to determine whether a therapy disclosed herein has been therapeutically effective for a subject in need thereof.

In particular embodiments, conclusions are drawn based on whether a sample value is statistically significantly different or not statistically significantly different from a reference level. A measure is not statistically significantly different if the difference is within a level that would be expected to occur based on chance alone. In contrast, a statistically significant difference or increase is one that is greater than what would be expected to occur by chance alone. Statistical significance or lack thereof can be determined by any of various methods well-known in the art. An example of a commonly used measure of statistical significance is the p-value. The p-value represents the probability of obtaining a given result equivalent to a particular data point, where the data point is the result of random chance alone. A result is often considered significant (not random chance) at a p-value less than or equal to 0.05. In particular embodiments, a sample value is “comparable to” a reference level derived from a normal control population if the sample value and the reference level are not statistically significantly different.

In particular embodiments, binding domain molecules disclosed herein can be used as standard controls. A “standard control” refers to a sample, measurement, or value that serves as a reference, usually a known reference, for comparison to a test sample, measurement, or value. For example, a known quantity of T antigens within a sample or a known quantity of antibodies that bind the T antigen can be used as a standard control. In particular embodiments, an established sample serving as a standard control provides an average amount of T antigen of the Merkel polyomavirus within the sample for an average healthy person.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (xviii) Exemplary Embodiments

1. An antibody or fragment thereof that binds a T antigen of the Merkel cell polyomavirus including a binding domain of antibody 1C10, 3B09, 1B09, 1G04, 2H04, W056-03, W056-04, W056-05, W056-06, W056-08, W056-09, W056-10, Z156-02, Z156-03, Z249-01, Z249-02, Z249-03, Z249-04, Z249-05, Z249-06, Z308-03, Z308-04, Z308-05, Z504-03, Z504-04, Z504-06, Z504-07, Z513-01, Z513-02, Z538-03, Z573-03, Z582-03, Z582-14, Z582-15, Z082-U1, Z082-U5, Z082-U6, W056-01, W056-02, W917-01, W917-02, W917-03, Z504-01, Z504-02, Z538-01, Z538-02, Z573-01, or Z573-2 according to North, Kabat, IMGT, Chothia, or Contact.
2. The antibody or fragment thereof of embodiment 1, wherein 1C10 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 57, the CDRH2 includes the sequence as set forth in SEQ ID NO: 58, the CDRH3 includes the sequence as set forth in SEQ ID NO: 59, the CDRL1 includes the sequence as set forth in SEQ ID NO: 60, the CDRL2 includes the sequence as set forth in SEQ ID NO: 61, and the CDRL3 includes the sequence as set forth in SEQ ID NO: 62, according to North;
   wherein the CDRH1 includes the sequence as set forth in SEQ ID NO: 63, the CDRH2 includes the sequence as set forth in SEQ ID NO: 64, the CDRH3 includes the sequence as set forth in SEQ ID NO: 65, the CDRL1 includes the sequence as set forth in SEQ ID NO: 60, the CDRL2 includes the sequence as set forth in SEQ ID NO: 66, and the CDRL3 includes the sequence as set forth in SEQ ID NO: 62, according to Kabat;
   wherein the CDRH1 includes the sequence as set forth in SEQ ID NO: 67, the CDRH2 includes the sequence as set forth in SEQ ID NO: 68, the CDRH3 includes the sequence as set forth in SEQ ID NO: 59, the CDRL1 includes the sequence as set forth in SEQ ID NO: 69, the CDRL2 includes the sequence KDT, and the CDRL3 includes the sequence as set forth in SEQ ID NO: 62, according to IMGT;
   wherein the CDRH1 includes the sequence as set forth in SEQ ID NO: 70, the CDRH2 includes the sequence as set forth in SEQ ID NO: 71, the CDRH3 includes the sequence as set forth in SEQ ID NO: 65, the CDRL1 includes the sequence as set forth in SEQ ID NO: 60, the CDRL2 includes the sequence as set forth in SEQ ID NO: 66, and the CDRL3 includes the sequence as set forth in SEQ ID NO: 62, according to Chothia; or
   wherein the CDRH1 includes the sequence as set forth in SEQ ID NO: 72, the CDRH2 includes the sequence as set forth in SEQ ID NO: 71, the CDRH3 includes the sequence as set forth in SEQ ID NO: 73, the CDRL1 includes the sequence as set forth in SEQ ID NO: 74, the CDRL2 includes the sequence as set forth in SEQ ID NO: 75, and the CDRL3 includes the sequence as set forth in SEQ ID NO: 76, according to Contact.
3. The antibody or fragment thereof of embodiment 1, wherein 3B09 includes a heavy chain variable region including a CDRH1, CDRH2, and CDRH3 and a light chain variable region including a CDRL1, CDRL2, and CDRL3;
   wherein the CDRH1 includes the sequence as set forth in SEQ ID NO: 79, the CDRH2 includes the sequence as set forth in SEQ ID NO: 80, the CDRH3 includes the sequence as set forth in SEQ ID NO: 81, the CDRL1 includes the sequence as set forth in SEQ ID NO: 82, the CDRL2 includes the sequence as set forth in SEQ ID NO: 83, the CDRL3 includes the sequence as set forth in SEQ ID NO: 84, according to North;
   wherein the CDRH1 includes the sequence as set forth in SEQ ID NO: 85, the CDRH2 includes the sequence as set forth in SEQ ID NO: 86, the CDRH3 includes the sequence as set forth in SEQ ID NO: 87, the CDRL1 includes the sequence as set forth in SEQ ID NO: 82, the CDRL2 includes the sequence as set forth in SEQ ID NO: 88, the CDRL3 includes the sequence as set forth in SEQ ID NO: 84, according to Kabat;
   wherein the CDRH1 includes the sequence as set forth in SEQ ID NO: 89, the CDRH2 includes the sequence as set forth in SEQ ID NO: 90, the CDRH3 includes the sequence as set forth in SEQ ID NO: 81, the CDRL1 includes the sequence as set forth in SEQ ID NO: 91, the CDRL2 includes the sequence DVH, the CDRL3 includes the sequence as set forth in SEQ ID NO: 84, according to IMGT;
   wherein the CDRH1 includes the sequence as set forth in SEQ ID NO: 92, the CDRH2 includes the sequence as set forth in SEQ ID NO: 93, the CDRH3 includes the sequence as set forth in SEQ ID NO: 87, the CDRL1 includes the sequence as set forth in SEQ ID NO: 82, the CDRL2 includes the sequence as set forth in SEQ ID NO: 88, the CDRL3 includes the sequence as set forth in SEQ ID NO: 84, according Chothia; or
   wherein the CDRH1 includes the sequence as set forth in SEQ ID NO: 94, the CDRH2 includes the sequence as set forth in SEQ ID NO: 95, the CDRH3 includes the sequence as set forth in SEQ ID NO: 96, the CDRL1 includes the sequence as set forth in SEQ ID NO: 97, the CDRL2 includes the sequence as set forth in SEQ ID NO: 98, the CDRL3 includes the sequence as set forth in SEQ ID NO: 99, according to Contact.
4. The antibody or fragment thereof of embodiment 1, wherein 1B09 includes a heavy chain variable region including a CDRH1, CDRH2, and CDRH3 and a light chain variable region including a CDRL1, CDRL2, and CDRL3;
   wherein the CDRH1 includes the sequence as set forth in SEQ ID NO: 102, the CDRH2 includes the sequence as set forth in SEQ ID NO: 103, the CDRH3 includes the sequence as set forth in SEQ ID NO: 104, the CDRL1 includes the sequence as set forth in SEQ ID NO: 105, the CDRL2 includes the sequence as set forth in SEQ ID NO: 106, the CDRL3 includes the sequence as set forth in SEQ ID NO: 107, according to North;
   wherein the CDRH1 includes the sequence as set forth in SEQ ID NO: 108, the CDRH2 includes the sequence as set forth in SEQ ID NO: 109, the CDRH3 includes the sequence as set forth in SEQ ID NO: 110, the CDRL1 includes the sequence as set forth in SEQ ID NO: 105, the CDRL2 includes the sequence as set forth in SEQ ID NO: 111, the CDRL3 includes the sequence as set forth in SEQ ID NO: 107, according to Kabat;
   wherein the CDRH1 includes the sequence as set forth in SEQ ID NO: 112, the CDRH2 includes the sequence as set forth in SEQ ID NO: 113, the CDRH3 includes the sequence as set forth in SEQ ID NO: 104, the CDRL1 includes the sequence as set forth in SEQ ID NO: 114, the CDRL2 includes the sequence DVS, the CDRL3 includes the sequence as set forth in SEQ ID NO: 107, according to IMGT;
   wherein the CDRH1 includes the sequence as set forth in SEQ ID NO: 115, the CDRH2 includes the sequence as set forth in SEQ ID NO: 116, the CDRH3 includes the sequence as set forth in SEQ ID NO: 110, the CDRL1 includes the sequence as set forth in SEQ ID NO: 105, the CDRL2 includes the sequence as set forth in SEQ ID NO: 111, the CDRL3 includes the sequence as set forth in SEQ ID NO: 107, according to Chothia; or
   wherein the CDRH1 includes the sequence as set forth in SEQ ID NO: 117, the CDRH2 includes the sequence as set forth in SEQ ID NO: 118, the CDRH3 includes the sequence as set forth in SEQ ID NO: 119, the CDRL1 includes the sequence as set forth in SEQ ID NO: 120, the CDRL2 includes the sequence as set forth in SEQ ID NO: 121, the CDRL3 includes the sequence as set forth in SEQ ID NO: 122, according to Contact.
5. The antibody or fragment thereof of embodiment 1, wherein 1G04 includes a heavy chain variable region including a CDRH1, CDRH2, and CDRH3 and a light chain variable region including a CDRL1, CDRL2, and CDRL3;
   wherein the CDRH1 includes the sequence as set forth in SEQ ID NO: 125, the CDRH2 includes the sequence as set forth in SEQ ID NO: 126, the CDRH3 includes the sequence as set forth in SEQ ID NO: 127, the CDRL1 includes the sequence as set forth in SEQ ID NO: 128, according to North;
   wherein the CDRL2 includes the sequence as set forth in SEQ ID NO: 129, the CDRL3 includes the sequence as set forth in SEQ ID NO: 130, the CDRH1 includes the sequence as set forth in SEQ ID NO: 131, the CDRH2 includes the sequence as set forth in SEQ ID NO: 132, the CDRH3 includes the sequence as set forth in SEQ ID NO: 133, the CDRL1 includes the sequence as set forth in SEQ ID NO: 128, the CDRL2 includes the sequence as set forth in SEQ ID NO: 134, the CDRL3 includes the sequence as set forth in SEQ ID NO: 130, according to Kabat;
   wherein the CDRH1 includes the sequence as set forth in SEQ ID NO: 135, the CDRH2 includes the sequence as set forth in SEQ ID NO: 136, the CDRH3 includes the sequence as set forth in SEQ ID NO: 127, the CDRL1 includes the sequence as set forth in SEQ ID NO: 137, the CDRL2 includes the sequence LAS, the CDRL3 includes the sequence as set forth in SEQ ID NO: 130, according to IMGT;
   wherein the CDRH1 includes the sequence as set forth in SEQ ID NO: 138, the CDRH2 includes the sequence as set forth in SEQ ID NO: 139, the CDRH3 includes the sequence as set forth in SEQ ID NO: 133, the CDRL1 includes the sequence as set forth in SEQ ID NO: 128, the CDRL2 includes the sequence as set forth in SEQ ID NO: 134, the CDRL3 includes the sequence as set forth in SEQ ID NO: 130, according to Chothia; or
   wherein the CDRH1 includes the sequence as set forth in SEQ ID NO: 140, the CDRH2 includes the sequence as set forth in SEQ ID NO: 141, the CDRH3 includes the sequence as set forth in SEQ ID NO: 142, the CDRL1 includes the sequence as set forth in SEQ ID NO: 143, the CDRL2 includes the sequence as set forth in SEQ ID NO: 144, the CDRL3 includes the sequence as set forth in SEQ ID NO: 145, according to Contact.
6. The antibody or fragment thereof of embodiment 1, wherein 2H04 includes a heavy chain variable region including a CDRH1, CDRH2, and CDRH3 and a light chain variable region including a CDRL1, CDRL2, and CDRL3;
   wherein the CDRH1 includes the sequence as set forth in SEQ ID NO: 148, the CDRH2 includes the sequence as set forth in SEQ ID NO: 149, the CDRH3 includes the sequence as set forth in SEQ ID NO: 150, the CDRL1 includes the sequence as set forth in SEQ ID NO: 151, the CDRL2 includes the sequence as set forth in SEQ ID NO: 152, the CDRL3 includes the sequence as set forth in SEQ ID NO: 153, according to North;
   wherein the CDRH1 includes the sequence as set forth in SEQ ID NO: 154, the CDRH2 includes the sequence as set forth in SEQ ID NO: 155, the CDRH3 includes the sequence as set forth in SEQ ID NO: 156, the CDRL1 includes the sequence as set forth in SEQ ID NO: 151, the CDRL2 includes the sequence as set forth in SEQ ID NO: 157, the CDRL3 includes the sequence as set forth in SEQ ID NO: 153, according to Kabat;
   wherein the CDRH1 includes the sequence as set forth in SEQ ID NO: 158, the CDRH2 includes the sequence as set forth in SEQ ID NO: 159, the CDRH3 includes the sequence as set forth in SEQ ID NO: 150, the CDRL1 includes the sequence as set forth in SEQ ID NO: 160, the CDRL2 includes the sequence DDR, the CDRL3 includes the sequence as set forth in SEQ ID NO: 153, according to IMGT;
   wherein the CDRH1 includes the sequence as set forth in SEQ ID NO: 161, the CDRH2 includes the sequence as set forth in SEQ ID NO: 162, the CDRH3 includes the sequence as set forth in SEQ ID NO: 156, the CDRL1 includes the sequence as set forth in SEQ ID NO: 151, the CDRL2 includes the sequence as set forth in SEQ ID NO: 157, the CDRL3 includes the sequence as set forth in SEQ ID NO: 153, according to Chothia; or
   wherein the CDRH1 includes the sequence as set forth in SEQ ID NO: 163, the CDRH2 includes the sequence as set forth in SEQ ID NO: 164, the CDRH3 includes the sequence as set forth in SEQ ID NO: 165, the CDRL1 includes the sequence as set forth in SEQ ID NO: 166, the CDRL2 includes the sequence as set forth in SEQ ID NO: 167, the CDRL3 includes the sequence as set forth in SEQ ID NO: 168, according to Contact.
7. The antibody or fragment thereof of embodiment 1, wherein W056-03 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 250, the CDRH2 includes the sequence as set forth in SEQ ID NO: 251, the CDRH3 includes the sequence as set forth in SEQ ID NO: 252, the CDRL1 includes the sequence as set forth in SEQ ID NO: 253, the CDRL2 includes the sequence as set forth in SEQ ID NO: 254, and the CDRL3 includes the sequence as set forth in SEQ ID NO: 255, according to North;
   wherein the CDRH1 includes the sequence as set forth in SEQ ID NO: 256, the CDRH2 includes the sequence as set forth in SEQ ID NO: 257, the CDRH3 includes the sequence as set forth in SEQ ID NO: 258, the CDRL1 includes the sequence as set forth in SEQ ID NO: 253, the CDRL2 includes the sequence as set forth in SEQ ID NO: 259, and the CDRL3 includes the sequence as set forth in SEQ ID NO: 255, according to Kabat;
   wherein the CDRH1 includes the sequence as set forth in SEQ ID NO: 260, the CDRH2 includes the sequence as set forth in SEQ ID NO: 261, the CDRH3 includes the sequence as set forth in SEQ ID NO: 252, the CDRL1 includes the sequence as set forth in SEQ ID NO: 262, the CDRL2 includes the sequence GN, and the CDRL3 includes the sequence as set forth in SEQ ID NO: 255, according to IMGT;
   wherein the CDRH1 includes the sequence as set forth in SEQ ID NO: 263, the CDRH2 includes the sequence as set forth in SEQ ID NO: 264, the CDRH3 includes the sequence as set forth in SEQ ID NO: 258, the CDRL1 includes the sequence as set forth in SEQ ID NO: 253, the CDRL2 includes the sequence as set forth in SEQ ID NO: 259, and the CDRL3 includes the sequence as set forth in SEQ ID NO: 255, according to Chothia; or
   wherein the CDRH1 includes the sequence as set forth in SEQ ID NO: 265, the CDRH2 includes the sequence as set forth in SEQ ID NO: 266, the CDRH3 includes the sequence as set forth in SEQ ID NO: 267, the CDRL1 includes the sequence as set forth in SEQ ID NO: 268, the CDRL2 includes the sequence as set forth in SEQ ID NO: 269, and the CDRL3 includes the sequence as set forth in SEQ ID NO: 270, according to Contact.
8. The antibody or fragment thereof of embodiment 7, wherein the CDRH1 is encoded by the sequence as set forth in SEQ ID NO: 991, the CDRH2 is encoded by the sequence as set forth in SEQ ID NO: 992, the CDRH3 is encoded by the sequence as set forth in SEQ ID NO: 993, the CDRL1 is encoded by the sequence as set forth in SEQ ID NO: 994, the CDRL2 is encoded by the sequence GGTAACAGT, the CDRL3 is encoded by the sequence as set forth in SEQ ID NO: 996.
9. The antibody or fragment thereof of embodiment 1, wherein W056-04 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 271, the CDRH2 includes the sequence as set forth in SEQ ID NO: 272, the CDRH3 includes the sequence as set forth in SEQ ID NO: 273, the CDRL1 includes the sequence as set forth in SEQ ID NO: 274, the CDRL2 includes the sequence as set forth in SEQ ID NO: 275, and the CDRL3 includes the sequence as set forth in SEQ ID NO: 276, according to North;
   wherein the CDRH1 includes the sequence as set forth in SEQ ID NO: 277, the CDRH2 includes the sequence as set forth in SEQ ID NO: 278, the CDRH3 includes the sequence as set forth in SEQ ID NO: 279, the CDRL1 includes the sequence as set forth in SEQ ID NO: 274, the CDRL2 includes the sequence as set forth in SEQ ID NO: 280, and the CDRL3 includes the sequence as set forth in SEQ ID NO: 276, according to Kabat;
   wherein the CDRH1 includes the sequence as set forth in SEQ ID NO: 281, the CDRH2 includes the sequence as set forth in SEQ ID NO: 282, the CDRH3 includes the sequence as set forth in SEQ ID NO: 273, the CDRL1 includes the sequence as set forth in SEQ ID NO: 283, the CDRL2 includes the sequence EV, and the CDRL3 includes the sequence as set forth in SEQ ID NO: 276, according to IMGT;
   wherein the CDRH1 includes the sequence as set forth in SEQ ID NO: 284 the CDRH2 includes the sequence as set forth in SEQ ID NO: 285, the CDRH3 includes the sequence as set forth in SEQ ID NO: 279, the CDRL1 includes the sequence as set forth in SEQ ID NO: 274, the CDRL2 includes the sequence as set forth in SEQ ID NO: 280, and the CDRL3 includes the sequence as set forth in SEQ ID NO: 276, according to Chothia; or
   wherein the CDRH1 includes the sequence as set forth in SEQ ID NO: 286, the CDRH2 includes the sequence as set forth in SEQ ID NO: 287, the CDRH3 includes the sequence as set forth in SEQ ID NO: 288, the CDRL1 includes the sequence as set forth in SEQ ID NO: 289, the CDRL2 includes the sequence as set forth in SEQ ID NO: 290, and the CDRL3 includes the sequence as set forth in SEQ ID NO: 291, according to Contact.
10. The antibody or fragment thereof of embodiment 9, wherein the CDRH1 is encoded by the sequence as set forth in SEQ ID NO: 997, the CDRH2 is encoded by the sequence as set forth in SEQ ID NO: 998, the CDRH3 is encoded by the sequence as set forth in SEQ ID NO: 999, the CDRL1 is encoded by the sequence as set forth in SEQ ID NO: 1000, the CDRL2 is encoded by the sequence GAGGTCAGT, the CDRL3 is encoded by the sequence as set forth in SEQ ID NO: 1002.
11. The antibody or fragment thereof of embodiment 1, wherein W056-05 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 292, the CDRH2 includes the sequence as set forth in SEQ ID NO: 293, the CDRH3 includes the sequence as set forth in SEQ ID NO: 294, the CDRL1 includes the sequence as set forth in SEQ ID NO: 295, the CDRL2 includes the sequence as set forth in SEQ ID NO: 296, and the CDRL3 includes the sequence as set forth in SEQ ID NO: 297, according to North;
   wherein the CDRH1 includes the sequence as set forth in SEQ ID NO: 298, the CDRH2 includes the sequence as set forth in SEQ ID NO: 299, the CDRH3 includes the sequence as set forth in SEQ ID NO: 300, the CDRL1 includes the sequence as set forth in SEQ ID NO: 295, the CDRL2 includes the sequence as set forth in SEQ ID NO: 301, and the CDRL3 includes the sequence as set forth in SEQ ID NO: 297, according to Kabat;
   wherein the CDRH1 includes the sequence as set forth in SEQ ID NO: 302, the CDRH2 includes the sequence as set forth in SEQ ID NO: 303, the CDRH3 includes the sequence as set forth in SEQ ID NO: 294, the CDRL1 includes the sequence as set forth in SEQ ID NO: 304, the CDRL2 includes the sequence AA, and the CDRL3 includes the sequence as set forth in SEQ ID NO: 297, according to IMGT;
   wherein the CDRH1 includes the sequence as set forth in SEQ ID NO: 305 the CDRH2 includes the sequence as set forth in SEQ ID NO: 306, the CDRH3 includes the sequence as set forth in SEQ ID NO: 300, the CDRL1 includes the sequence as set forth in SEQ ID NO: 295, the CDRL2 includes the sequence as set forth in SEQ ID NO: 301, and the CDRL3 includes the sequence as set forth in SEQ ID NO: 297, according to Chothia; or
   wherein the CDRH1 includes the sequence as set forth in SEQ ID NO: 307, the CDRH2 includes the sequence as set forth in SEQ ID NO: 308, the CDRH3 includes the sequence as set forth in SEQ ID NO: 309, the CDRL1 includes the sequence as set forth in SEQ ID NO: 310, the CDRL2 includes the sequence as set forth in SEQ ID NO: 311, and the CDRL3 includes the sequence as set forth in SEQ ID NO: 312, according to Contact.
12. The antibody or fragment thereof of embodiment 11, wherein the CDRH1 is encoded by the sequence as set forth in SEQ ID NO: 1003, the CDRH2 is encoded by the sequence as set forth in SEQ ID NO: 1004, the CDRH3 is encoded by the sequence as set forth in SEQ ID NO: 1005, the CDRL1 is encoded by the sequence as set forth in SEQ ID NO: 1006, the CDRL2 is encoded by the sequence GCTGCATCC, the CDRL3 is encoded by the sequence as set forth in SEQ ID NO: 1008.
13. The antibody or fragment thereof of embodiment 1, wherein W056-06 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 313, the CDRH2 includes the sequence as set forth in SEQ ID NO: 314, the CDRH3 includes the sequence as set forth in SEQ ID NO: 315, the CDRL1 includes the sequence as set forth in SEQ ID NO: 316, the CDRL2 includes the sequence as set forth in SEQ ID NO: 317, and the CDRL3 includes the sequence as set forth in SEQ ID NO: 318, according to North;
   wherein the CDRH1 includes the sequence as set forth in SEQ ID NO: 319, the CDRH2 includes the sequence as set forth in SEQ ID NO: 320, the CDRH3 includes the sequence as set forth in SEQ ID NO: 321, the CDRL1 includes the sequence as set forth in SEQ ID NO: 316, the CDRL2 includes the sequence as set forth in SEQ ID NO: 322, and the CDRL3 includes the sequence as set forth in SEQ ID NO: 318, according to Kabat;
   wherein the CDRH1 includes the sequence as set forth in SEQ ID NO: 323, the CDRH2 includes the sequence as set forth in SEQ ID NO: 324, the CDRH3 includes the sequence as set forth in SEQ ID NO: 315, the CDRL1 includes the sequence as set forth in SEQ ID NO: 325, the CDRL2 includes the sequence EA, and the CDRL3 includes the sequence as set forth in SEQ ID NO: 318, according to IMGT;
   wherein the CDRH1 includes the sequence as set forth in SEQ ID NO: 326, the CDRH2 includes the sequence as set forth in SEQ ID NO: 327, the CDRH3 includes the sequence as set forth in SEQ ID NO: 321, the CDRL1 includes the sequence as set forth in SEQ ID NO: 316, the CDRL2 includes the sequence as set forth in SEQ ID NO: 322, and the CDRL3 includes the sequence as set forth in SEQ ID NO: 318, according to Chothia; or
   wherein the CDRH1 includes the sequence as set forth in SEQ ID NO: 323, the CDRH2 includes the sequence as set forth in SEQ ID NO: 324, the CDRH3 includes the sequence as set forth in SEQ ID NO: 325, the CDRL1 includes the sequence as set forth in SEQ ID NO: 326, the CDRL2 includes the sequence as set forth in SEQ ID NO: 327, and the CDRL3 includes the sequence as set forth in SEQ ID NO: 335, according to Contact.
14. The antibody or fragment thereof of embodiment 13, wherein the CDRH1 is encoded by the sequence as set forth in SEQ ID NO: 1009, the CDRH2 is encoded by the sequence as set forth in SEQ ID NO: 1010, the CDRH3 is encoded by the sequence as set forth in SEQ ID NO: 1111, the CDRL1 is encoded by the sequence as set forth in SEQ ID NO: 1112, the CDRL2 is encoded by the sequence GAGGCGTCC, the CDRL3 is encoded by the sequence as set forth in SEQ ID NO: 1114.
15. The antibody or fragment thereof of embodiment 1, wherein W056-08 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 271, the CDRH2 includes the sequence as set forth in SEQ ID NO: 272, the CDRH3 includes the sequence as set forth in SEQ ID NO: 273, the CDRL1 includes the sequence as set forth in SEQ ID NO: 274, the CDRL2 includes the sequence as set forth in SEQ ID NO: 275, and the CDRL3 includes the sequence as set forth in SEQ ID NO: 276, according to North;
   wherein the CDRH1 includes the sequence as set forth in SEQ ID NO: 277, the CDRH2 includes the sequence as set forth in SEQ ID NO: 278, the CDRH3 includes the sequence as set forth in SEQ ID NO: 279, the CDRL1 includes the sequence as set forth in SEQ ID NO: 274, the CDRL2 includes the sequence as set forth in SEQ ID NO: 280, and the CDRL3 includes the sequence as set forth in SEQ ID NO: 276, according to Kabat;
   wherein the CDRH1 includes the sequence as set forth in SEQ ID NO: 281, the CDRH2 includes the sequence as set forth in SEQ ID NO: 282, the CDRH3 includes the sequence as set forth in SEQ ID NO: 273, the CDRL1 includes the sequence as set forth in SEQ ID NO: 283, the CDRL2 includes the sequence EV, and the CDRL3 includes the sequence as set forth in SEQ ID NO: 318 according to IMGT;
   wherein the CDRH1 includes the sequence as set forth in SEQ ID NO: 284, the CDRH2 includes the sequence as set forth in SEQ ID NO: 285, the CDRH3 includes the sequence as set forth in SEQ ID NO: 279, the CDRL1 includes the sequence as set forth in SEQ ID NO: 274, the CDRL2 includes the sequence as set forth in SEQ ID NO: 280, and the CDRL3 includes the sequence as set forth in SEQ ID NO: 276, according to Chothia; or
   wherein the CDRH1 includes the sequence as set forth in SEQ ID NO: 286, the CDRH2 includes the sequence as set forth in SEQ ID NO: 287, the CDRH3 includes the sequence as set forth in SEQ ID NO: 288, the CDRL1 includes the sequence as set forth in SEQ ID NO: 289, the CDRL2 includes the sequence as set forth in SEQ ID NO: 290, and the CDRL3 includes the sequence as set forth in SEQ ID NO: 291, according to Contact.
16. The antibody or fragment thereof of embodiment 13, wherein the CDRH1 is encoded by the sequence as set forth in SEQ ID NO: 997, the CDRH2 is encoded by the sequence as set forth in SEQ ID NO: 998, the CDRH3 is encoded by the sequence as set forth in SEQ ID NO: 999, the CDRL1 is encoded by the sequence as set forth in SEQ ID NO: 1000, the CDRL2 is encoded by the sequence GAGGTCAGT, the CDRL3 is encoded by the sequence as set forth in SEQ ID NO: 1002.
17. The antibody or fragment thereof of embodiment 1, wherein W056-09 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 328, the CDRH2 includes the sequence as set forth in SEQ ID NO: 314, the CDRH3 includes the sequence as set forth in SEQ ID NO: 315, the CDRL1 includes the sequence as set forth in SEQ ID NO: 329, the CDRL2 includes the sequence as set forth in SEQ ID NO: 317, and the CDRL3 includes the sequence as set forth in SEQ ID NO: 318, according to North;
   wherein the CDRH1 includes the sequence as set forth in SEQ ID NO: 319, the CDRH2 includes the sequence as set forth in SEQ ID NO: 320, the CDRH3 includes the sequence as set forth in SEQ ID NO: 321, the CDRL1 includes the sequence as set forth in SEQ ID NO: 329, the CDRL2 includes the sequence as set forth in SEQ ID NO: 322, and the CDRL3 includes the sequence as set forth in SEQ ID NO: 318, according to Kabat;
   wherein the CDRH1 includes the sequence as set forth in SEQ ID NO: 330, the CDRH2 includes the sequence as set forth in SEQ ID NO: 324, the CDRH3 includes the sequence as set forth in SEQ ID NO: 315, the CDRL1 includes the sequence as set forth in SEQ ID NO: 331, the CDRL2 includes the sequence EV, and the CDRL3 includes the sequence as set forth in SEQ ID NO: 318 according to IMGT;
   wherein the CDRH1 includes the sequence as set forth in SEQ ID NO: 332, the CDRH2 includes the sequence as set forth in SEQ ID NO: 327, the CDRH3 includes the sequence as set forth in SEQ ID NO: 321, the CDRL1 includes the sequence as set forth in SEQ ID NO: 329, the CDRL2 includes the sequence as set forth in SEQ ID NO: 322, and the CDRL3 includes the sequence as set forth in SEQ ID NO: 318, according to Chothia; or
   wherein the CDRH1 includes the sequence as set forth in SEQ ID NO: 333, the CDRH2 includes the sequence as set forth in SEQ ID NO: 324, the CDRH3 includes the sequence as set forth in SEQ ID NO: 325, the CDRL1 includes the sequence as set forth in SEQ ID NO: 334, the CDRL2 includes the sequence as set forth in SEQ ID NO: 327, and the CDRL3 includes the sequence as set forth in SEQ ID NO: 335, according to Contact.
18. The antibody or fragment thereof of embodiment 17, wherein the CDRH1 is encoded by the sequence as set forth in SEQ ID NO: 1115, the CDRH2 is encoded by the sequence as set forth in SEQ ID NO: 1116, the CDRH3 is encoded by the sequence as set forth in SEQ ID NO: 1111, the CDRL1 is encoded by the sequence as set forth in SEQ ID NO: 1117, the CDRL2 is encoded by the sequence GAGGCGTCC, the CDRL3 is encoded by the sequence as set forth in SEQ ID NO: 1118.
19. The antibody or fragment thereof of embodiment 1, wherein W056-10 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 336, the CDRH2 includes the sequence as set forth in SEQ ID NO: 337, the CDRH3 includes the sequence as set forth in SEQ ID NO: 338, the CDRL1 includes the sequence as set forth in SEQ ID NO: 339, the CDRL2 includes the sequence as set forth in SEQ ID NO: 340, and the CDRL3 includes the sequence as set forth in SEQ ID NO: 341, according to North;
   wherein the CDRH1 includes the sequence as set forth in SEQ ID NO: 342, the CDRH2 includes the sequence as set forth in SEQ ID NO: 343, the CDRH3 includes the sequence as set forth in SEQ ID NO: 344, the CDRL1 includes the sequence as set forth in SEQ ID NO: 339, the CDRL2 includes the sequence as set forth in SEQ ID NO: 345, and the CDRL3 includes the sequence as set forth in SEQ ID NO: 341, according to Kabat;
   wherein the CDRH1 includes the sequence as set forth in SEQ ID NO: 346, the CDRH2 includes the sequence as set forth in SEQ ID NO: 347, the CDRH3 includes the sequence as set forth in SEQ ID NO: 338, the CDRL1 includes the sequence as set forth in SEQ ID NO: 348, the CDRL2 includes the sequence AA, and the CDRL3 includes the sequence as set forth in SEQ ID NO: 341 according to IMGT;
   wherein the CDRH1 includes the sequence as set forth in SEQ ID NO: 349, the CDRH2 includes the sequence as set forth in SEQ ID NO: 350, the CDRH3 includes the sequence as set forth in SEQ ID NO: 344, the CDRL1 includes the sequence as set forth in SEQ ID NO: 339, the CDRL2 includes the sequence as set forth in SEQ ID NO: 345, and the CDRL3 includes the sequence as set forth in SEQ ID NO: 341, according to Chothia; or
   wherein the CDRH1 includes the sequence as set forth in SEQ ID NO: 351, the CDRH2 includes the sequence as set forth in SEQ ID NO: 352, the CDRH3 includes the sequence as set forth in SEQ ID NO: 353, the CDRL1 includes the sequence as set forth in SEQ ID NO: 354, the CDRL2 includes the sequence as set forth in SEQ ID NO: 355, and the CDRL3 includes the sequence as set forth in SEQ ID NO: 356, according to Contact.
20. The antibody or fragment thereof of embodiment 19, wherein the CDRH1 is encoded by the sequence as set forth in SEQ ID NO: 1119, the CDRH2 is encoded by the sequence as set forth in SEQ ID NO: 1120, the CDRH3 is encoded by the sequence as set forth in SEQ ID NO: 1121, the CDRL1 is encoded by the sequence as set forth in SEQ ID NO: 1122, the CDRL2 is encoded by the sequence GCTGCATCC, the CDRL3 is encoded by the sequence as set forth in SEQ ID NO: 1123.
21. The antibody or fragment thereof of embodiment 1, wherein Z156-02 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 357, the CDRH2 includes the sequence as set forth in SEQ ID NO: 358, the CDRH3 includes the sequence as set forth in SEQ ID NO: 359, the CDRL1 includes the sequence as set forth in SEQ ID NO: 360, the CDRL2 includes the sequence as set forth in SEQ ID NO: 361, and the CDRL3 includes the sequence as set forth in SEQ ID NO: 362, according to North;
   wherein the CDRH1 includes the sequence as set forth in SEQ ID NO: 363, the CDRH2 includes the sequence as set forth in SEQ ID NO: 364, the CDRH3 includes the sequence as set forth in SEQ ID NO: 365, the CDRL1 includes the sequence as set forth in SEQ ID NO: 360, the CDRL2 includes the sequence as set forth in SEQ ID NO: 366, and the CDRL3 includes the sequence as set forth in SEQ ID NO: 362, according to Kabat;
   wherein the CDRH1 includes the sequence as set forth in SEQ ID NO: 367, the CDRH2 includes the sequence as set forth in SEQ ID NO: 368, the CDRH3 includes the sequence as set forth in SEQ ID NO: 359, the CDRL1 includes the sequence as set forth in SEQ ID NO: 369, the CDRL2 includes the sequence KD, and the CDRL3 includes the sequence as set forth in SEQ ID NO: 362 according to IMGT;
   wherein the CDRH1 includes the sequence as set forth in SEQ ID NO: 370, the CDRH2 includes the sequence as set forth in SEQ ID NO: 371, the CDRH3 includes the sequence as set forth in SEQ ID NO: 365, the CDRL1 includes the sequence as set forth in SEQ ID NO: 360, the CDRL2 includes the sequence as set forth in SEQ ID NO: 366, and the CDRL3 includes the sequence as set forth in SEQ ID NO: 362, according to Chothia; or
   wherein the CDRH1 includes the sequence as set forth in SEQ ID NO: 372 the CDRH2 includes the sequence as set forth in SEQ ID NO: 373, the CDRH3 includes the sequence as set forth in SEQ ID NO: 374 the CDRL1 includes the sequence as set forth in SEQ ID NO: 74, the CDRL2 includes the sequence as set forth in SEQ ID NO: 375, and the CDRL3 includes the sequence as set forth in SEQ ID NO: 376, according to Contact.
22. The antibody or fragment thereof of embodiment 21, wherein the CDRH1 is encoded by the sequence as set forth in SEQ ID NO: 1124, the CDRH2 is encoded by the sequence as set forth in SEQ ID NO: 1125, the CDRH3 is encoded by the sequence as set forth in SEQ ID NO: 1126, the CDRL1 is encoded by the sequence as set forth in SEQ ID NO: 1127, the CDRL2 is encoded by the sequence AAAGACAGT, the CDRL3 is encoded by the sequence as set forth in SEQ ID NO: 1129.
23. The antibody or fragment thereof of embodiment 1, wherein Z156-03 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 377, the CDRH2 includes the sequence as set forth in SEQ ID NO: 378, the CDRH3 includes the sequence as set forth in SEQ ID NO: 379, the CDRL1 includes the sequence as set forth in SEQ ID NO: 380, the CDRL2 includes the sequence as set forth in SEQ ID NO: 361, and the CDRL3 includes the sequence as set forth in SEQ ID NO: 381, according to North;
   wherein the CDRH1 includes the sequence as set forth in SEQ ID NO: 85, the CDRH2 includes the sequence as set forth in SEQ ID NO: 382, the CDRH3 includes the sequence as set forth in SEQ ID NO: 383, the CDRL1 includes the sequence as set forth in SEQ ID NO: 380, the CDRL2 includes the sequence as set forth in SEQ ID NO: 366, and the CDRL3 includes the sequence as set forth in SEQ ID NO: 381, according to Kabat;
   wherein the CDRH1 includes the sequence as set forth in SEQ ID NO: 384, the CDRH2 includes the sequence as set forth in SEQ ID NO: 385, the CDRH3 includes the sequence as set forth in SEQ ID NO: 379, the CDRL1 includes the sequence as set forth in SEQ ID NO: 386, the CDRL2 includes the sequence KD, and the CDRL3 includes the sequence as set forth in SEQ ID NO: 381 according to IMGT;
   wherein the CDRH1 includes the sequence as set forth in SEQ ID NO: 387, the CDRH2 includes the sequence as set forth in SEQ ID NO: 388, the CDRH3 includes the sequence as set forth in SEQ ID NO: 383, the CDRL1 includes the sequence as set forth in SEQ ID NO: 380, the CDRL2 includes the sequence as set forth in SEQ ID NO: 366, and the CDRL3 includes the sequence as set forth in SEQ ID NO: 381, according to Chothia; or
   wherein the CDRH1 includes the sequence as set forth in SEQ ID NO: 94 the CDRH2 includes the sequence as set forth in SEQ ID NO: 389, the CDRH3 includes the sequence as set forth in SEQ ID NO: 390, the CDRL1 includes the sequence as set forth in SEQ ID NO: 391, the CDRL2 includes the sequence as set forth in SEQ ID NO: 375, and the CDRL3 includes the sequence as set forth in SEQ ID NO: 392, according to Contact.
24. The antibody or fragment thereof of embodiment 23, wherein the CDRH1 is encoded by the sequence as set forth in SEQ ID NO: 1124, the CDRH2 is encoded by the sequence as set forth in SEQ ID NO: 1125, the CDRH3 is encoded by the sequence as set forth in SEQ ID NO: 1126, the CDRL1 is encoded by the sequence as set forth in SEQ ID NO: 1130, the CDRL2 is encoded by the sequence as set forth in SEQ ID NO: 1131, the CDRL3 is encoded by the sequence as set forth in SEQ ID NO: 1132.
25. The antibody or fragment thereof of embodiment 1, wherein Z249-01 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 393, the CDRH2 includes the sequence as set forth in SEQ ID NO: 394, the CDRH3 includes the sequence as set forth in SEQ ID NO: 395, the CDRL1 includes the sequence as set forth in SEQ ID NO: 396, the CDRL2 includes the sequence as set forth in SEQ ID NO: 397, and the CDRL3 includes the sequence as set forth in SEQ ID NO: 398, according to North;
   wherein the CDRH1 includes the sequence as set forth in SEQ ID NO: 399, the CDRH2 includes the sequence as set forth in SEQ ID NO: 400, the CDRH3 includes the sequence as set forth in SEQ ID NO: 401, the CDRL1 includes the sequence as set forth in SEQ ID NO: 396, the CDRL2 includes the sequence as set forth in SEQ ID NO: 402, and the CDRL3 includes the sequence as set forth in SEQ ID NO: 398, according to Kabat;
   wherein the CDRH1 includes the sequence as set forth in SEQ ID NO: 403, the CDRH2 includes the sequence as set forth in SEQ ID NO: 404, the CDRH3 includes the sequence as set forth in SEQ ID NO: 395, the CDRL1 includes the sequence as set forth in SEQ ID NO: 405, the CDRL2 includes the sequence KD, and the CDRL3 includes the sequence as set forth in SEQ ID NO: 398 according to IMGT;
   wherein the CDRH1 includes the sequence as set forth in SEQ ID NO: 406, the CDRH2 includes the sequence as set forth in SEQ ID NO: 407, the CDRH3 includes the sequence as set forth in SEQ ID NO: 401, the CDRL1 includes the sequence as set forth in SEQ ID NO: 396, the CDRL2 includes the sequence as set forth in SEQ ID NO: 402, and the CDRL3 includes the sequence as set forth in SEQ ID NO: 398, according to Chothia; or
   wherein the CDRH1 includes the sequence as set forth in SEQ ID NO: 408, the CDRH2 includes the sequence as set forth in SEQ ID NO: 409, the CDRH3 includes the sequence as set forth in SEQ ID NO: 410, the CDRL1 includes the sequence as set forth in SEQ ID NO: 411, the CDRL2 includes the sequence as set forth in SEQ ID NO: 412, and the CDRL3 includes the sequence as set forth in SEQ ID NO: 413, according to Contact.
26. The antibody or fragment thereof of embodiment 25, wherein the CDRH1 is encoded by the sequence as set forth in SEQ ID NO: 1133, the CDRH2 is encoded by the sequence as set forth in SEQ ID NO: 1134, the CDRH3 is encoded by the sequence as set forth in SEQ ID NO: 1135, the CDRL1 is encoded by the sequence as set forth in SEQ ID NO: 1136, the CDRL2 is encoded by the sequence AAAGACAAT, the CDRL3 is encoded by the sequence as set forth in SEQ ID NO: 1138.
27. The antibody or fragment thereof of embodiment 1, wherein Z249-02 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 414, the CDRH2 includes the sequence as set forth in SEQ ID NO: 415, the CDRH3 includes the sequence as set forth in SEQ ID NO: 416, the CDRL1 includes the sequence as set forth in SEQ ID NO: 417, the CDRL2 includes the sequence as set forth in SEQ ID NO: 418, and the CDRL3 includes the sequence as set forth in SEQ ID NO: 419, according to North;
   wherein the CDRH1 includes the sequence as set forth in SEQ ID NO: 420, the CDRH2 includes the sequence as set forth in SEQ ID NO: 421, the CDRH3 includes the sequence as set forth in SEQ ID NO: 422, the CDRL1 includes the sequence as set forth in SEQ ID NO: 417, the CDRL2 includes the sequence as set forth in SEQ ID NO: 423, and the CDRL3 includes the sequence as set forth in SEQ ID NO: 419, according to Kabat;
   wherein the CDRH1 includes the sequence as set forth in SEQ ID NO: 424, the CDRH2 includes the sequence as set forth in SEQ ID NO: 425, the CDRH3 includes the sequence as set forth in SEQ ID NO: 416, the CDRL1 includes the sequence as set forth in SEQ ID NO: 426, the CDRL2 includes the sequence GA, and the CDRL3 includes the sequence as set forth in SEQ ID NO: 419 according to IMGT;
   wherein the CDRH1 includes the sequence as set forth in SEQ ID NO: 427, the CDRH2 includes the sequence as set forth in SEQ ID NO: 428, the CDRH3 includes the sequence as set forth in SEQ ID NO: 422, the CDRL1 includes the sequence as set forth in SEQ ID NO: 417, the CDRL2 includes the sequence as set forth in SEQ ID NO: 423, and the CDRL3 includes the sequence as set forth in SEQ ID NO: 419, according to Chothia; or
   wherein the CDRH1 includes the sequence as set forth in SEQ ID NO: 429, the CDRH2 includes the sequence as set forth in SEQ ID NO: 430, the CDRH3 includes the sequence as set forth in SEQ ID NO: 431, the CDRL1 includes the sequence as set forth in SEQ ID NO: 432, the CDRL2 includes the sequence as set forth in SEQ ID NO: 433, and the CDRL3 includes the sequence as set forth in SEQ ID NO: 434, according to Contact.
28. The antibody or fragment thereof of embodiment 27, wherein the CDRH1 is encoded by the sequence as set forth in SEQ ID NO: 1139, the CDRH2 is encoded by the sequence as set forth in SEQ ID NO: 1140, the CDRH3 is encoded by the sequence as set forth in SEQ ID NO: 1141, the CDRL1 is encoded by the sequence as set forth in SEQ ID NO: 1142, the CDRL2 is encoded by the sequence GGTGCATCC, the CDRL3 is encoded by the sequence as set forth in SEQ ID NO: 1144.
29. The antibody or fragment thereof of embodiment 1, wherein Z249-03 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 435, the CDRH2 includes the sequence as set forth in SEQ ID NO: 436, the CDRH3 includes the sequence as set forth in SEQ ID NO: 437, the CDRL1 includes the sequence as set forth in SEQ ID NO: 438, the CDRL2 includes the sequence as set forth in SEQ ID NO: 439, and the CDRL3 includes the sequence as set forth in SEQ ID NO: 440, according to North;
   wherein the CDRH1 includes the sequence as set forth in SEQ ID NO: 441, the CDRH2 includes the sequence as set forth in SEQ ID NO: 442, the CDRH3 includes the sequence as set forth in SEQ ID NO: 443, the CDRL1 includes the sequence as set forth in SEQ ID NO: 438, the CDRL2 includes the sequence as set forth in SEQ ID NO: 444, and the CDRL3 includes the sequence as set forth in SEQ ID NO: 440, according to Kabat;
   wherein the CDRH1 includes the sequence as set forth in SEQ ID NO: 445, the CDRH2 includes the sequence as set forth in SEQ ID NO: 446, the CDRH3 includes the sequence as set forth in SEQ ID NO: 437, the CDRL1 includes the sequence as set forth in SEQ ID NO: 447, the CDRL2 includes the sequence DV, and the CDRL3 includes the sequence as set forth in SEQ ID NO: 440 according to IMGT;
   wherein the CDRH1 includes the sequence as set forth in SEQ ID NO: 448, the CDRH2 includes the sequence as set forth in SEQ ID NO: 449, the CDRH3 includes the sequence as set forth in SEQ ID NO: 443, the CDRL1 includes the sequence as set forth in SEQ ID NO: 438, the CDRL2 includes the sequence as set forth in SEQ ID NO: 444, and the CDRL3 includes the sequence as set forth in SEQ ID NO: 440, according to Chothia; or
   wherein the CDRH1 includes the sequence as set forth in SEQ ID NO: 450, the CDRH2 includes the sequence as set forth in SEQ ID NO: 451, the CDRH3 includes the sequence as set forth in SEQ ID NO: 452, the CDRL1 includes the sequence as set forth in SEQ ID NO: 289, the CDRL2 includes the sequence as set forth in SEQ ID NO: 453, and the CDRL3 includes the sequence as set forth in SEQ ID NO: 454, according to Contact.
30. The antibody or fragment thereof of embodiment 29, wherein the CDRH1 is encoded by the sequence as set forth in SEQ ID NO: 1145, the CDRH2 is encoded by the sequence as set forth in SEQ ID NO: 1146, the CDRH3 is encoded by the sequence as set forth in SEQ ID NO: 1147, the CDRL1 is encoded by the sequence as set forth in SEQ ID NO: 1148, the CDRL2 is encoded by the sequence GATGTCACT, the CDRL3 is encoded by the sequence as set forth in SEQ ID NO: 1150.
31. The antibody or fragment thereof of embodiment 1, wherein Z249-04 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 455, the CDRH2 includes the sequence as set forth in SEQ ID NO: 456, the CDRH3 includes the sequence as set forth in SEQ ID NO: 457, the CDRL1 includes the sequence as set forth in SEQ ID NO: 458, the CDRL2 includes the sequence as set forth in SEQ ID NO: 361, and the CDRL3 includes the sequence as set forth in SEQ ID NO: 459, according to North;
   wherein the CDRH1 includes the sequence as set forth in SEQ ID NO: 460, the CDRH2 includes the sequence as set forth in SEQ ID NO: 461, the CDRH3 includes the sequence as set forth in SEQ ID NO: 462, the CDRL1 includes the sequence as set forth in SEQ ID NO: 458, the CDRL2 includes the sequence as set forth in SEQ ID NO: 366, and the CDRL3 includes the sequence as set forth in SEQ ID NO: 459, according to Kabat;
   wherein the CDRH1 includes the sequence as set forth in SEQ ID NO: 463, the CDRH2 includes the sequence as set forth in SEQ ID NO: 464, the CDRH3 includes the sequence as set forth in SEQ ID NO: 457, the CDRL1 includes the sequence as set forth in SEQ ID NO: 465, the CDRL2 includes the sequence KD, and the CDRL3 includes the sequence as set forth in SEQ ID NO: 459 according to IMGT;
   wherein the CDRH1 includes the sequence as set forth in SEQ ID NO: 466, the CDRH2 includes the sequence as set forth in SEQ ID NO: 467, the CDRH3 includes the sequence as set forth in SEQ ID NO: 462, the CDRL1 includes the sequence as set forth in SEQ ID NO: 458, the CDRL2 includes the sequence as set forth in SEQ ID NO: 366, and the CDRL3 includes the sequence as set forth in SEQ ID NO: 459, according to Chothia; or
   wherein the CDRH1 includes the sequence as set forth in SEQ ID NO: 468, the CDRH2 includes the sequence as set forth in SEQ ID NO: 469, the CDRH3 includes the sequence as set forth in SEQ ID NO: 470, the CDRL1 includes the sequence as set forth in SEQ ID NO: 471, the CDRL2 includes the sequence as set forth in SEQ ID NO: 472, and the CDRL3 includes the sequence as set forth in SEQ ID NO: 473, according to Contact.
32. The antibody or fragment thereof of embodiment 31, wherein the CDRH1 is encoded by the sequence as set forth in SEQ ID NO: 1151, the CDRH2 is encoded by the sequence as set forth in SEQ ID NO: 1152, the CDRH3 is encoded by the sequence as set forth in SEQ ID NO: 1153, the CDRL1 is encoded by the sequence as set forth in SEQ ID NO: 1154, the CDRL2 is encoded by the sequence AAGGACAGC, the CDRL3 is encoded by the sequence as set forth in SEQ ID NO: 1156.
33. The antibody or fragment thereof of embodiment 1, wherein Z249-05 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 474, the CDRH2 includes the sequence as set forth in SEQ ID NO: 475, the CDRH3 includes the sequence as set forth in SEQ ID NO: 476, the CDRL1 includes the sequence as set forth in SEQ ID NO: 477, the CDRL2 includes the sequence as set forth in SEQ ID NO: 61, and the CDRL3 includes the sequence as set forth in SEQ ID NO: 478, according to North;
   wherein the CDRH1 includes the sequence as set forth in SEQ ID NO: 479, the CDRH2 includes the sequence as set forth in SEQ ID NO: 480, the CDRH3 includes the sequence as set forth in SEQ ID NO: 481, the CDRL1 includes the sequence as set forth in SEQ ID NO: 477, the CDRL2 includes the sequence as set forth in SEQ ID NO: 41, and the CDRL3 includes the sequence as set forth in SEQ ID NO: 478, according to Kabat;
   wherein the CDRH1 includes the sequence as set forth in SEQ ID NO: 482, the CDRH2 includes the sequence as set forth in SEQ ID NO: 483, the CDRH3 includes the sequence as set forth in SEQ ID NO: 476, the CDRL1 includes the sequence as set forth in SEQ ID NO: 484, the CDRL2 includes the sequence KD, and the CDRL3 includes the sequence as set forth in SEQ ID NO: 478 according to IMGT;
   wherein the CDRH1 includes the sequence as set forth in SEQ ID NO: 485, the CDRH2 includes the sequence as set forth in SEQ ID NO: 486, the CDRH3 includes the sequence as set forth in SEQ ID NO: 481, the CDRL1 includes the sequence as set forth in SEQ ID NO: 477, the CDRL2 includes the sequence as set forth in SEQ ID NO: 66, and the CDRL3 includes the sequence as set forth in SEQ ID NO: 478, according to Chothia; or
   wherein the CDRH1 includes the sequence as set forth in SEQ ID NO: 487, the CDRH2 includes the sequence as set forth in SEQ ID NO: 488, the CDRH3 includes the sequence as set forth in SEQ ID NO: 489, the CDRL1 includes the sequence as set forth in SEQ ID NO: 490, the CDRL2 includes the sequence as set forth in SEQ ID NO: 491, and the CDRL3 includes the sequence as set forth in SEQ ID NO: 491, according to Contact.
34. The antibody or fragment thereof of embodiment 33, wherein the CDRH1 is encoded by the sequence as set forth in SEQ ID NO: 1157, the CDRH2 is encoded by the sequence as set forth in SEQ ID NO: 1158, the CDRH3 is encoded by the sequence as set forth in SEQ ID NO: 1159, the CDRL1 is encoded by the sequence as set forth in SEQ ID NO: 1160, the CDRL2 is encoded by the sequence AAAGACACT, the CDRL3 is encoded by the sequence as set forth in SEQ ID NO: 1162.
35. The antibody or fragment thereof of embodiment 1, wherein Z249-06 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 414, the CDRH2 includes the sequence as set forth in SEQ ID NO: 415, the CDRH3 includes the sequence as set forth in SEQ ID NO: 416, the CDRL1 includes the sequence as set forth in SEQ ID NO: 417, the CDRL2 includes the sequence as set forth in SEQ ID NO: 418, and the CDRL3 includes the sequence as set forth in SEQ ID NO: 419, according to North;
   wherein the CDRH1 includes the sequence as set forth in SEQ ID NO: 420, the CDRH2 includes the sequence as set forth in SEQ ID NO: 421, the CDRH3 includes the sequence as set forth in SEQ ID NO: 422, the CDRL1 includes the sequence as set forth in SEQ ID NO: 417, the CDRL2 includes the sequence as set forth in SEQ ID NO: 423, and the CDRL3 includes the sequence as set forth in SEQ ID NO: 419, according to Kabat;
   wherein the CDRH1 includes the sequence as set forth in SEQ ID NO: 424, the CDRH2 includes the sequence as set forth in SEQ ID NO: 425, the CDRH3 includes the sequence as set forth in SEQ ID NO: 416, the CDRL1 includes the sequence as set forth in SEQ ID NO: 426, the CDRL2 includes the sequence GA, and the CDRL3 includes the sequence as set forth in SEQ ID NO: 419 according to IMGT;
   wherein the CDRH1 includes the sequence as set forth in SEQ ID NO: 427, the CDRH2 includes the sequence as set forth in SEQ ID NO: 428, the CDRH3 includes the sequence as set forth in SEQ ID NO: 422, the CDRL1 includes the sequence as set forth in SEQ ID NO: 417, the CDRL2 includes the sequence as set forth in SEQ ID NO: 423, and the CDRL3 includes the sequence as set forth in SEQ ID NO: 419, according to Chothia; or
   wherein the CDRH1 includes the sequence as set forth in SEQ ID NO: 429, the CDRH2 includes the sequence as set forth in SEQ ID NO: 430, the CDRH3 includes the sequence as set forth in SEQ ID NO: 431, the CDRL1 includes the sequence as set forth in SEQ ID NO: 432, the CDRL2 includes the sequence as set forth in SEQ ID NO: 433, and the CDRL3 includes the sequence as set forth in SEQ ID NO: 434, according to Contact.
36. The antibody or fragment thereof of embodiment 35, wherein the CDRH1 is encoded by the sequence as set forth in SEQ ID NO: 1139, the CDRH2 is encoded by the sequence as set forth in SEQ ID NO: 1140, the CDRH3 is encoded by the sequence as set forth in SEQ ID NO: 1141, the CDRL1 is encoded by the sequence as set forth in SEQ ID NO: 1142, the CDRL2 is encoded by the sequence GGTGCATCC, the CDRL3 is encoded by the sequence as set forth in SEQ ID NO: 1144.
37. The antibody or fragment thereof of embodiment 1, wherein Z308-03 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 493, the CDRH2 includes the sequence as set forth in SEQ ID NO: 494, the CDRH3 includes the sequence as set forth in SEQ ID NO: 495, the CDRL1 includes the sequence as set forth in SEQ ID NO: 496, the CDRL2 includes the sequence as set forth in SEQ ID NO: 497, and the CDRL3 includes the sequence as set forth in SEQ ID NO: 498, according to North;
   wherein the CDRH1 includes the sequence as set forth in SEQ ID NO: 499, the CDRH2 includes the sequence as set forth in SEQ ID NO: 500, the CDRH3 includes the sequence as set forth in SEQ ID NO: 501, the CDRL1 includes the sequence as set forth in SEQ ID NO: 496, the CDRL2 includes the sequence as set forth in SEQ ID NO: 502, and the CDRL3 includes the sequence as set forth in SEQ ID NO: 498, according to Kabat;
   wherein the CDRH1 includes the sequence as set forth in SEQ ID NO: 503, the CDRH2 includes the sequence as set forth in SEQ ID NO: 504, the CDRH3 includes the sequence as set forth in SEQ ID NO: 495, the CDRL1 includes the sequence as set forth in SEQ ID NO: 505, the CDRL2 includes the sequence SA, and the CDRL3 includes the sequence as set forth in SEQ ID NO: 498 according to IMGT;
   wherein the CDRH1 includes the sequence as set forth in SEQ ID NO: 506, the CDRH2 includes the sequence as set forth in SEQ ID NO: 507, the CDRH3 includes the sequence as set forth in SEQ ID NO: 501, the CDRL1 includes the sequence as set forth in SEQ ID NO: 496, the CDRL2 includes the sequence as set forth in SEQ ID NO: 502, and the CDRL3 includes the sequence as set forth in SEQ ID NO: 498, according to Chothia; or
   wherein the CDRH1 includes the sequence as set forth in SEQ ID NO: 508, the CDRH2 includes the sequence as set forth in SEQ ID NO: 509, the CDRH3 includes the sequence as set forth in SEQ ID NO: 510, the CDRL1 includes the sequence as set forth in SEQ ID NO: 511, the CDRL2 includes the sequence as set forth in SEQ ID NO: 512, and the CDRL3 includes the sequence as set forth in SEQ ID NO: 513, according to Contact.
38. The antibody or fragment thereof of embodiment 37, wherein the CDRH1 is encoded by the sequence as set forth in SEQ ID NO: 1163, the CDRH2 is encoded by the sequence as set forth in SEQ ID NO: 1164, the CDRH3 is encoded by the sequence as set forth in SEQ ID NO: 1165, the CDRL1 is encoded by the sequence as set forth in SEQ ID NO: 1166, the CDRL2 is encoded by the sequence AGTGCATCC, the CDRL3 is encoded by the sequence as set forth in SEQ ID NO: 1168.
39. The antibody or fragment thereof of embodiment 1, wherein Z308-04 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 514, the CDRH2 includes the sequence as set forth in SEQ ID NO: 515, the CDRH3 includes the sequence as set forth in SEQ ID NO: 516, the CDRL1 includes the sequence as set forth in SEQ ID NO: 517, the CDRL2 includes the sequence as set forth in SEQ ID NO: 518, and the CDRL3 includes the sequence as set forth in SEQ ID NO: 519, according to North;
   wherein the CDRH1 includes the sequence as set forth in SEQ ID NO: 520, the CDRH2 includes the sequence as set forth in SEQ ID NO: 521, the CDRH3 includes the sequence as set forth in SEQ ID NO: 522, the CDRL1 includes the sequence as set forth in SEQ ID NO: 517, the CDRL2 includes the sequence as set forth in SEQ ID NO: 523, and the CDRL3 includes the sequence as set forth in SEQ ID NO: 519, according to Kabat;
   wherein the CDRH1 includes the sequence as set forth in SEQ ID NO: 524, the CDRH2 includes the sequence as set forth in SEQ ID NO: 525, the CDRH3 includes the sequence as set forth in SEQ ID NO: 516, the CDRL1 includes the sequence as set forth in SEQ ID NO: 526, the CDRL2 includes the sequence TA, and the CDRL3 includes the sequence as set forth in SEQ ID NO: 519 according to IMGT;
   wherein the CDRH1 includes the sequence as set forth in SEQ ID NO: 527, the CDRH2 includes the sequence as set forth in SEQ ID NO: 528, the CDRH3 includes the sequence as set forth in SEQ ID NO: 522, the CDRL1 includes the sequence as set forth in SEQ ID NO: 517, the CDRL2 includes the sequence as set forth in SEQ ID NO: 523, and the CDRL3 includes the sequence as set forth in SEQ ID NO: 519, according to Chothia; or
   wherein the CDRH1 includes the sequence as set forth in SEQ ID NO: 529, the CDRH2 includes the sequence as set forth in SEQ ID NO: 530, the CDRH3 includes the sequence as set forth in SEQ ID NO: 531, the CDRL1 includes the sequence as set forth in SEQ ID NO: 532, the CDRL2 includes the sequence as set forth in SEQ ID NO: 533, and the CDRL3 includes the sequence as set forth in SEQ ID NO: 534, according to Contact.
40. The antibody or fragment thereof of embodiment 39, wherein the CDRH1 is encoded by the sequence as set forth in SEQ ID NO: 1169, the CDRH2 is encoded by the sequence as set forth in SEQ ID NO: 1170, the CDRH3 is encoded by the sequence as set forth in SEQ ID NO: 1171, the CDRL1 is encoded by the sequence as set forth in SEQ ID NO: 1172, the CDRL2 is encoded by the sequence ACTGCGTCC, the CDRL3 is encoded by the sequence as set forth in SEQ ID NO: 1174.
41. The antibody or fragment thereof of embodiment 1, wherein Z308-05 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 535, the CDRH2 includes the sequence as set forth in SEQ ID NO: 272, the CDRH3 includes the sequence as set forth in SEQ ID NO: 536, the CDRL1 includes the sequence as set forth in SEQ ID NO: 537, the CDRL2 includes the sequence as set forth in SEQ ID NO: 497, and the CDRL3 includes the sequence as set forth in SEQ ID NO: 538, according to North;
   wherein the CDRH1 includes the sequence as set forth in SEQ ID NO: 539, the CDRH2 includes the sequence as set forth in SEQ ID NO: 540, the CDRH3 includes the sequence as set forth in SEQ ID NO: 541, the CDRL1 includes the sequence as set forth in SEQ ID NO: 537, the CDRL2 includes the sequence as set forth in SEQ ID NO: 502, and the CDRL3 includes the sequence as set forth in SEQ ID NO: 538, according to Kabat;
   wherein the CDRH1 includes the sequence as set forth in SEQ ID NO: 542, the CDRH2 includes the sequence as set forth in SEQ ID NO: 282, the CDRH3 includes the sequence as set forth in SEQ ID NO: 536, the CDRL1 includes the sequence as set forth in SEQ ID NO: 542, the CDRL2 includes the sequence SA, and the CDRL3 includes the sequence as set forth in SEQ ID NO: 538 according to IMGT;
   wherein the CDRH1 includes the sequence as set forth in SEQ ID NO: 543, the CDRH2 includes the sequence as set forth in SEQ ID NO: 285, the CDRH3 includes the sequence as set forth in SEQ ID NO: 541, the CDRL1 includes the sequence as set forth in SEQ ID NO: 537, the CDRL2 includes the sequence as set forth in SEQ ID NO: 502, and the CDRL3 includes the sequence as set forth in SEQ ID NO: 538, according to Chothia; or
   wherein the CDRH1 includes the sequence as set forth in SEQ ID NO: 544, the CDRH2 includes the sequence as set forth in SEQ ID NO: 287, the CDRH3 includes the sequence as set forth in SEQ ID NO: 545, the CDRL1 includes the sequence as set forth in SEQ ID NO: 546, the CDRL2 includes the sequence as set forth in SEQ ID NO: 512, and the CDRL3 includes the sequence as set forth in SEQ ID NO: 547, according to Contact.
42. The antibody or fragment thereof of embodiment 41, wherein the CDRH1 is encoded by the sequence as set forth in SEQ ID NO: 1175, the CDRH2 is encoded by the sequence as set forth in SEQ ID NO: 998, the CDRH3 is encoded by the sequence as set forth in SEQ ID NO: 1176, the CDRL1 is encoded by the sequence as set forth in SEQ ID NO: 1177, the CDRL2 is encoded by the sequence AGTGCATCC, the CDRL3 is encoded by the sequence as set forth in SEQ ID NO: 1178.
43. The antibody or fragment thereof of embodiment 1, wherein Z504-03 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 548, the CDRH2 includes the sequence as set forth in SEQ ID NO: 549, the CDRH3 includes the sequence as set forth in SEQ ID NO: 550, the CDRL1 includes the sequence as set forth in SEQ ID NO: 551, the CDRL2 includes the sequence as set forth in SEQ ID NO: 552, and the CDRL3 includes the sequence as set forth in SEQ ID NO: 1308, according to North;
   wherein the CDRH1 includes the sequence as set forth in SEQ ID NO: 553, the CDRH2 includes the sequence as set forth in SEQ ID NO: 554, the CDRH3 includes the sequence as set forth in SEQ ID NO: 555, the CDRL1 includes the sequence as set forth in SEQ ID NO: 551, the CDRL2 includes the sequence as set forth in SEQ ID NO: 556, and the CDRL3 includes the sequence as set forth in SEQ ID NO: 1308, according to Kabat;
   wherein the CDRH1 includes the sequence as set forth in SEQ ID NO: 557, the CDRH2 includes the sequence as set forth in SEQ ID NO: 558, the CDRH3 includes the sequence as set forth in SEQ ID NO: 550, the CDRL1 includes the sequence as set forth in SEQ ID NO: 559, the CDRL2 includes the sequence GA, and the CDRL3 includes the sequence as set forth in SEQ ID NO: 1308 according to IMGT;
   wherein the CDRH1 includes the sequence as set forth in SEQ ID NO: 560 the CDRH2 includes the sequence as set forth in SEQ ID NO: 561, the CDRH3 includes the sequence as set forth in SEQ ID NO: 555, the CDRL1 includes the sequence as set forth in SEQ ID NO: 551, the CDRL2 includes the sequence as set forth in SEQ ID NO: 556, and the CDRL3 includes the sequence as set forth in SEQ ID NO: 1308, according to Chothia; or
   wherein the CDRH1 includes the sequence as set forth in SEQ ID NO: 562, the CDRH2 includes the sequence as set forth in SEQ ID NO: 563, the CDRH3 includes the sequence as set forth in SEQ ID NO: 564, the CDRL1 includes the sequence as set forth in SEQ ID NO: 565, the CDRL2 includes the sequence as set forth in SEQ ID NO: 566, and the CDRL3 includes the sequence as set forth in SEQ ID NO: 567, according to Contact.
44. The antibody or fragment thereof of embodiment 43, wherein the CDRH1 is encoded by the sequence as set forth in SEQ ID NO: 1179, the CDRH2 is encoded by the sequence as set forth in SEQ ID NO: 1180, the CDRH3 is encoded by the sequence as set forth in SEQ ID NO: 1181, the CDRL1 is encoded by the sequence as set forth in SEQ ID NO: 1182, the CDRL2 is encoded by the sequence GGTGCATCC, the CDRL3 is encoded by the sequence as set forth in SEQ ID NO: 1183.
45. The antibody or fragment thereof of embodiment 1, wherein Z504-03 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 568, the CDRH2 includes the sequence as set forth in SEQ ID NO: 569, the CDRH3 includes the sequence as set forth in SEQ ID NO: 570, the CDRL1 includes the sequence as set forth in SEQ ID NO: 571, the CDRL2 includes the sequence as set forth in SEQ ID NO: 572, and the CDRL3 includes the sequence as set forth in SEQ ID NO: 573, according to North;
   wherein the CDRH1 includes the sequence as set forth in SEQ ID NO: 342, the CDRH2 includes the sequence as set forth in SEQ ID NO: 574, the CDRH3 includes the sequence as set forth in SEQ ID NO: 570, the CDRL1 includes the sequence as set forth in SEQ ID NO: 571, the CDRL2 includes the sequence as set forth in SEQ ID NO: 575, and the CDRL3 includes the sequence as set forth in SEQ ID NO: 573, according to Kabat;
   wherein the CDRH1 includes the sequence as set forth in SEQ ID NO: 576, the CDRH2 includes the sequence as set forth in SEQ ID NO: 577, the CDRH3 includes the sequence as set forth in SEQ ID NO: 570, the CDRL1 includes the sequence as set forth in SEQ ID NO: 578, the CDRL2 includes the sequence GA, and the CDRL3 includes the sequence as set forth in SEQ ID NO: 573 according to IMGT;
   wherein the CDRH1 includes the sequence as set forth in SEQ ID NO: 579, the CDRH2 includes the sequence as set forth in SEQ ID NO: 580, the CDRH3 includes the sequence as set forth in SEQ ID NO: 570, the CDRL1 includes the sequence as set forth in SEQ ID NO: 571, the CDRL2 includes the sequence as set forth in SEQ ID NO: 575, and the CDRL3 includes the sequence as set forth in SEQ ID NO: 573, according to Chothia; or
   wherein the CDRH1 includes the sequence as set forth in SEQ ID NO: 351, the CDRH2 includes the sequence as set forth in SEQ ID NO: 581, the CDRH3 includes the sequence as set forth in SEQ ID NO: 582, the CDRL1 includes the sequence as set forth in SEQ ID NO: 583, the CDRL2 includes the sequence as set forth in SEQ ID NO: 584, and the CDRL3 includes the sequence as set forth in SEQ ID NO: 585, according to Contact.
46. The antibody or fragment thereof of embodiment 45, wherein the CDRH1 is encoded by the sequence as set forth in SEQ ID NO: 1184, the CDRH2 is encoded by the sequence as set forth in SEQ ID NO: 1185, the CDRH3 is encoded by the sequence as set forth in SEQ ID NO: 1186, the CDRL1 is encoded by the sequence as set forth in SEQ ID NO: 1187, the CDRL2 is encoded by the sequence GGTGCATCC, the CDRL3 is encoded by the sequence as set forth in SEQ ID NO: 1188.
47. The antibody or fragment thereof of embodiment 1, wherein Z504-06 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 586, the CDRH2 includes the sequence as set forth in SEQ ID NO: 569, the CDRH3 includes the sequence as set forth in SEQ ID NO: 587, the CDRL1 includes the sequence as set forth in SEQ ID NO: 571, the CDRL2 includes the sequence as set forth in SEQ ID NO: 572, and the CDRL3 includes the sequence as set forth in SEQ ID NO: 588, according to North;
   wherein the CDRH1 includes the sequence as set forth in SEQ ID NO: 589, the CDRH2 includes the sequence as set forth in SEQ ID NO: 574, the CDRH3 includes the sequence as set forth in SEQ ID NO: 590, the CDRL1 includes the sequence as set forth in SEQ ID NO: 571, the CDRL2 includes the sequence as set forth in SEQ ID NO: 575, and the CDRL3 includes the sequence as set forth in SEQ ID NO: 588, according to Kabat;
   wherein the CDRH1 includes the sequence as set forth in SEQ ID NO: 591, the CDRH2 includes the sequence as set forth in SEQ ID NO: 577, the CDRH3 includes the sequence as set forth in SEQ ID NO: 587, the CDRL1 includes the sequence as set forth in SEQ ID NO: 578, the CDRL2 includes the sequence GA, and the CDRL3 includes the sequence as set forth in SEQ ID NO: 588 according to IMGT;
   wherein the CDRH1 includes the sequence as set forth in SEQ ID NO: 592, the CDRH2 includes the sequence as set forth in SEQ ID NO: 580, the CDRH3 includes the sequence as set forth in SEQ ID NO: 590, the CDRL1 includes the sequence as set forth in SEQ ID NO: 571, the CDRL2 includes the sequence as set forth in SEQ ID NO: 575, and the CDRL3 includes the sequence as set forth in SEQ ID NO: 588, according to Chothia; or
   wherein the CDRH1 includes the sequence as set forth in SEQ ID NO: 593, the CDRH2 includes the sequence as set forth in SEQ ID NO: 581, the CDRH3 includes the sequence as set forth in SEQ ID NO: 594, the CDRL1 includes the sequence as set forth in SEQ ID NO: 583, the CDRL2 includes the sequence as set forth in SEQ ID NO: 584, and the CDRL3 includes the sequence as set forth in SEQ ID NO: 595, according to Contact.
48. The antibody or fragment thereof of embodiment 47, wherein the CDRH1 is encoded by the sequence as set forth in SEQ ID NO: 1189, the CDRH2 is encoded by the sequence as set forth in SEQ ID NO: 1185, the CDRH3 is encoded by the sequence as set forth in SEQ ID NO: 1190, the CDRL1 is encoded by the sequence as set forth in SEQ ID NO: 1187, the CDRL2 is encoded by the sequence GGTGCATCC, the CDRL3 is encoded by the sequence as set forth in SEQ ID NO: 1191.
49. The antibody or fragment thereof of embodiment 1, wherein Z504-07 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 596, the CDRH2 includes the sequence as set forth in SEQ ID NO: 597, the CDRH3 includes the sequence as set forth in SEQ ID NO: 598, the CDRL1 includes the sequence as set forth in SEQ ID NO: 599, the CDRL2 includes the sequence as set forth in SEQ ID NO: 600, and the CDRL3 includes the sequence as set forth in SEQ ID NO: 601, according to North;
   wherein the CDRH1 includes the sequence as set forth in SEQ ID NO: 602, the CDRH2 includes the sequence as set forth in SEQ ID NO: 603, the CDRH3 includes the sequence as set forth in SEQ ID NO: 604, the CDRL1 includes the sequence as set forth in SEQ ID NO: 599, the CDRL2 includes the sequence as set forth in SEQ ID NO: 605, and the CDRL3 includes the sequence as set forth in SEQ ID NO: 601, according to Kabat;
   wherein the CDRH1 includes the sequence as set forth in SEQ ID NO: 606, the CDRH2 includes the sequence as set forth in SEQ ID NO: 607, the CDRH3 includes the sequence as set forth in SEQ ID NO: 604, the CDRL1 includes the sequence as set forth in SEQ ID NO: 608, the CDRL2 includes the sequence IT, and the CDRL3 includes the sequence as set forth in SEQ ID NO: 601 according to IMGT;
   wherein the CDRH1 includes the sequence as set forth in SEQ ID NO: 609, the CDRH2 includes the sequence as set forth in SEQ ID NO: 610, the CDRH3 includes the sequence as set forth in SEQ ID NO: 604, the CDRL1 includes the sequence as set forth in SEQ ID NO: 599, the CDRL2 includes the sequence as set forth in SEQ ID NO: 605, and the CDRL3 includes the sequence as set forth in SEQ ID NO: 601, according to Chothia; or
   wherein the CDRH1 includes the sequence as set forth in SEQ ID NO: 611, the CDRH2 includes the sequence as set forth in SEQ ID NO: 612, the CDRH3 includes the sequence as set forth in SEQ ID NO: 613, the CDRL1 includes the sequence as set forth in SEQ ID NO: 614, the CDRL2 includes the sequence as set forth in SEQ ID NO: 615, and the CDRL3 includes the sequence as set forth in SEQ ID NO: 616, according to Contact.
50. The antibody or fragment thereof of embodiment 49, wherein the CDRH1 is encoded by the sequence as set forth in SEQ ID NO: 1192, the CDRH2 is encoded by the sequence as set forth in SEQ ID NO: 1193, the CDRH3 is encoded by the sequence as set forth in SEQ ID NO: 1194, the CDRL1 is encoded by the sequence as set forth in SEQ ID NO: 1195, the CDRL2 is encoded by the sequence ACTACATCC, the CDRL3 is encoded by the sequence as set forth in SEQ ID NO: 1197.
51. The antibody or fragment thereof of embodiment 1, wherein Z513-01 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 617, the CDRH2 includes the sequence as set forth in SEQ ID NO: 618, the CDRH3 includes the sequence as set forth in SEQ ID NO: 619, the CDRL1 includes the sequence as set forth in SEQ ID NO: 620, the CDRL2 includes the sequence as set forth in SEQ ID NO: 621, and the CDRL3 includes the sequence as set forth in SEQ ID NO: 622, according to North;
   wherein the CDRH1 includes the sequence as set forth in SEQ ID NO: 623, the CDRH2 includes the sequence as set forth in SEQ ID NO: 624, the CDRH3 includes the sequence as set forth in SEQ ID NO: 625, the CDRL1 includes the sequence as set forth in SEQ ID NO: 620, the CDRL2 includes the sequence as set forth in SEQ ID NO: 626, and the CDRL3 includes the sequence as set forth in SEQ ID NO: 622, according to Kabat;
   wherein the CDRH1 includes the sequence as set forth in SEQ ID NO: 627, the CDRH2 includes the sequence as set forth in SEQ ID NO: 628, the CDRH3 includes the sequence as set forth in SEQ ID NO: 625, the CDRL1 includes the sequence as set forth in SEQ ID NO: 629, the CDRL2 includes the sequence VA, and the CDRL3 includes the sequence as set forth in SEQ ID NO: 622 according to IMGT;
   wherein the CDRH1 includes the sequence as set forth in SEQ ID NO: 630, the CDRH2 includes the sequence as set forth in SEQ ID NO: 631, the CDRH3 includes the sequence as set forth in SEQ ID NO: 625, the CDRL1 includes the sequence as set forth in SEQ ID NO: 620, the CDRL2 includes the sequence as set forth in SEQ ID NO: 626, and the CDRL3 includes the sequence as set forth in SEQ ID NO: 622, according to Chothia; or
   wherein the CDRH1 includes the sequence as set forth in SEQ ID NO: 632, the CDRH2 includes the sequence as set forth in SEQ ID NO: 633, the CDRH3 includes the sequence as set forth in SEQ ID NO: 634, the CDRL1 includes the sequence as set forth in SEQ ID NO: 635, the CDRL2 includes the sequence as set forth in SEQ ID NO: 636, and the CDRL3 includes the sequence as set forth in SEQ ID NO: 637, according to Contact.
52. The antibody or fragment thereof of embodiment 51, wherein the CDRH1 is encoded by the sequence as set forth in SEQ ID NO: 1198, the CDRH2 is encoded by the sequence as set forth in SEQ ID NO: 1199, the CDRH3 is encoded by the sequence as set forth in SEQ ID NO: 1200, the CDRL1 is encoded by the sequence as set forth in SEQ ID NO: 1201, the CDRL2 is encoded by the sequence as set forth in SEQ ID NO: 1201, the CDRL3 is encoded by the sequence GTTGCATCC.
53. The antibody or fragment thereof of embodiment 1, wherein Z513-01 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 638, the CDRH2 includes the sequence as set forth in SEQ ID NO: 639, the CDRH3 includes the sequence as set forth in SEQ ID NO: 640, the CDRL1 includes the sequence as set forth in SEQ ID NO: 641, the CDRL2 includes the sequence as set forth in SEQ ID NO: 642, and the CDRL3 includes the sequence as set forth in SEQ ID NO: 643, according to North;
   wherein the CDRH1 includes the sequence as set forth in SEQ ID NO: 644, the CDRH2 includes the sequence as set forth in SEQ ID NO: 645, the CDRH3 includes the sequence as set forth in SEQ ID NO: 646, the CDRL1 includes the sequence as set forth in SEQ ID NO: 641, the CDRL2 includes the sequence as set forth in SEQ ID NO: 647, and the CDRL3 includes the sequence as set forth in SEQ ID NO: 643, according to Kabat;
   wherein the CDRH1 includes the sequence as set forth in SEQ ID NO: 648, the CDRH2 includes the sequence as set forth in SEQ ID NO: 649, the CDRH3 includes the sequence as set forth in SEQ ID NO: 640, the CDRL1 includes the sequence as set forth in SEQ ID NO: 650, the CDRL2 includes the sequence AA, and the CDRL3 includes the sequence as set forth in SEQ ID NO: 643 according to IMGT;
   wherein the CDRH1 includes the sequence as set forth in SEQ ID NO: 651, the CDRH2 includes the sequence as set forth in SEQ ID NO: 652, the CDRH3 includes the sequence as set forth in SEQ ID NO: 646, the CDRL1 includes the sequence as set forth in SEQ ID NO: 641, the CDRL2 includes the sequence as set forth in SEQ ID NO: 647, and the CDRL3 includes the sequence as set forth in SEQ ID NO: 643, according to Chothia; or
   wherein the CDRH1 includes the sequence as set forth in SEQ ID NO: 653, the CDRH2 includes the sequence as set forth in SEQ ID NO: 654, the CDRH3 includes the sequence as set forth in SEQ ID NO: 655, the CDRL1 includes the sequence as set forth in SEQ ID NO: 656, the CDRL2 includes the sequence as set forth in SEQ ID NO: 657, and the CDRL3 includes the sequence as set forth in SEQ ID NO: 658, according to Contact.
54. The antibody or fragment thereof of embodiment 53, wherein the CDRH1 is encoded by the sequence as set forth in SEQ ID NO: 1204, the CDRH2 is encoded by the sequence as set forth in SEQ ID NO: 1205, the CDRH3 is encoded by the sequence as set forth in SEQ ID NO: 1206, the CDRL1 is encoded by the sequence as set forth in SEQ ID NO: 1207, the CDRL2 is encoded by the sequence GCTGCTTCC, the CDRL3 is encoded by the sequence as set forth in SEQ ID NO: 1209.
55. The antibody or fragment thereof of embodiment 1, wherein Z538-03 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 659, the CDRH2 includes the sequence as set forth in SEQ ID NO: 660, the CDRH3 includes the sequence as set forth in SEQ ID NO: 661, the CDRL1 includes the sequence as set forth in SEQ ID NO: 641, the CDRL2 includes the sequence as set forth in SEQ ID NO: 662, and the CDRL3 includes the sequence as set forth in SEQ ID NO: 663, according to North;
   wherein the CDRH1 includes the sequence as set forth in SEQ ID NO: 665, the CDRH2 includes the sequence as set forth in SEQ ID NO: 666, the CDRH3 includes the sequence as set forth in SEQ ID NO: 667, the CDRL1 includes the sequence as set forth in SEQ ID NO: 662, the CDRL2 includes the sequence as set forth in SEQ ID NO: 668, and the CDRL3 includes the sequence as set forth in SEQ ID NO: 664, according to Kabat;
   wherein the CDRH1 includes the sequence as set forth in SEQ ID NO: 669, the CDRH2 includes the sequence as set forth in SEQ ID NO: 670, the CDRH3 includes the sequence as set forth in SEQ ID NO: 661, the CDRL1 includes the sequence as set forth in SEQ ID NO: 671, the CDRL2 includes the sequence as set forth in SEQ ID NO: 672, and the CDRL3 includes the sequence as set forth in SEQ ID NO: 664 according to IMGT;
   wherein the CDRH1 includes the sequence as set forth in SEQ ID NO: 673, the CDRH2 includes the sequence as set forth in SEQ ID NO: 674, the CDRH3 includes the sequence as set forth in SEQ ID NO: 667, the CDRL1 includes the sequence as set forth in SEQ ID NO: 662, the CDRL2 includes the sequence as set forth in SEQ ID NO: 668, and the CDRL3 includes the sequence as set forth in SEQ ID NO: 664, according to Chothia; or
   wherein the CDRH1 includes the sequence as set forth in SEQ ID NO: 675, the CDRH2 includes the sequence as set forth in SEQ ID NO: 676, the CDRH3 includes the sequence as set forth in SEQ ID NO: 677, the CDRL1 includes the sequence as set forth in SEQ ID NO: 678, the CDRL2 includes the sequence as set forth in SEQ ID NO: 679, and the CDRL3 includes the sequence as set forth in SEQ ID NO: 680, according to Contact.
56. The antibody or fragment thereof of embodiment 55, wherein the CDRH1 is encoded by the sequence as set forth in SEQ ID NO: 1210, the CDRH2 is encoded by the sequence as set forth in SEQ ID NO: 1211, the CDRH3 is encoded by the sequence as set forth in SEQ ID NO: 1212, the CDRL1 is encoded by the sequence as set forth in SEQ ID NO: 1213, the CDRL2 is encoded by the sequence as set forth in SEQ ID NO: 1214, the CDRL3 is encoded by the sequence as set forth in SEQ ID NO: 1215.
57. The antibody or fragment thereof of embodiment 1, wherein Z573-03 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 681, the CDRH2 includes the sequence as set forth in SEQ ID NO: 682, the CDRH3 includes the sequence as set forth in SEQ ID NO: 683, the CDRL1 includes the sequence as set forth in SEQ ID NO: 684, the CDRL2 includes the sequence as set forth in SEQ ID NO: 361, and the CDRL3 includes the sequence as set forth in SEQ ID NO: 685, according to North;
   wherein the CDRH1 includes the sequence as set forth in SEQ ID NO: 686, the CDRH2 includes the sequence as set forth in SEQ ID NO: 687, the CDRH3 includes the sequence as set forth in SEQ ID NO: 688, the CDRL1 includes the sequence as set forth in SEQ ID NO: 684, the CDRL2 includes the sequence as set forth in SEQ ID NO: 366, and the CDRL3 includes the sequence as set forth in SEQ ID NO: 685, according to Kabat;
   wherein the CDRH1 includes the sequence as set forth in SEQ ID NO: 689, the CDRH2 includes the sequence as set forth in SEQ ID NO: 690, the CDRH3 includes the sequence as set forth in SEQ ID NO: 683, the CDRL1 includes the sequence as set forth in SEQ ID NO: 691, the CDRL2 includes the sequence KD, and the CDRL3 includes the sequence as set forth in SEQ ID NO: 685 according to IMGT;
   wherein the CDRH1 includes the sequence as set forth in SEQ ID NO: 692, the CDRH2 includes the sequence as set forth in SEQ ID NO: 693, the CDRH3 includes the sequence as set forth in SEQ ID NO: 688, the CDRL1 includes the sequence as set forth in SEQ ID NO: 684, the CDRL2 includes the sequence as set forth in SEQ ID NO: 366, and the CDRL3 includes the sequence as set forth in SEQ ID NO: 685, according to Chothia; or
   wherein the CDRH1 includes the sequence as set forth in SEQ ID NO: 694, the CDRH2 includes the sequence as set forth in SEQ ID NO: 695, the CDRH3 includes the sequence as set forth in SEQ ID NO: 696, the CDRL1 includes the sequence as set forth in SEQ ID NO: 697, the CDRL2 includes the sequence as set forth in SEQ ID NO: 375, and the CDRL3 includes the sequence as set forth in SEQ ID NO: 698, according to Contact.
58. The antibody or fragment thereof of embodiment 57, wherein the CDRH1 is encoded by the sequence as set forth in SEQ ID NO: 1216, the CDRH2 is encoded by the sequence as set forth in SEQ ID NO: 1217, the CDRH3 is encoded by the sequence as set forth in SEQ ID NO: 1218, the CDRL1 is encoded by the sequence as set forth in SEQ ID NO: 1219, the CDRL2 is encoded by the sequence as set forth in SEQ ID NO: 1220, the CDRL3 is encoded by the sequence as set forth in SEQ ID NO: 1221.
59. The antibody or fragment thereof of embodiment 1, wherein Z582-03 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 699, the CDRH2 includes the sequence as set forth in SEQ ID NO: 700, the CDRH3 includes the sequence as set forth in SEQ ID NO: 701, the CDRL1 includes the sequence as set forth in SEQ ID NO: 702, the CDRL2 includes the sequence as set forth in SEQ ID NO: 703, and the CDRL3 includes the sequence as set forth in SEQ ID NO: 704, according to North;
   wherein the CDRH1 includes the sequence as set forth in SEQ ID NO: 705, the CDRH2 includes the sequence as set forth in SEQ ID NO: 706, the CDRH3 includes the sequence as set forth in SEQ ID NO: 707, the CDRL1 includes the sequence as set forth in SEQ ID NO: 702, the CDRL2 includes the sequence as set forth in SEQ ID NO: 708, and the CDRL3 includes the sequence as set forth in SEQ ID NO: 704, according to Kabat;
   wherein the CDRH1 includes the sequence as set forth in SEQ ID NO: 709, the CDRH2 includes the sequence as set forth in SEQ ID NO: 710, the CDRH3 includes the sequence as set forth in SEQ ID NO: 701, the CDRL1 includes the sequence as set forth in SEQ ID NO: 711, the CDRL2 includes the sequence GA, and the CDRL3 includes the sequence as set forth in SEQ ID NO: 704 according to IMGT;
   wherein the CDRH1 includes the sequence as set forth in SEQ ID NO: 712, the CDRH2 includes the sequence as set forth in SEQ ID NO: 713, the CDRH3 includes the sequence as set forth in SEQ ID NO: 707, the CDRL1 includes the sequence as set forth in SEQ ID NO: 702, the CDRL2 includes the sequence as set forth in SEQ ID NO: 708, and the CDRL3 includes the sequence as set forth in SEQ ID NO: 704, according to Chothia; or
   wherein the CDRH1 includes the sequence as set forth in SEQ ID NO: 714, the CDRH2 includes the sequence as set forth in SEQ ID NO: 715, the CDRH3 includes the sequence as set forth in SEQ ID NO: 716, the CDRL1 includes the sequence as set forth in SEQ ID NO: 565, the CDRL2 includes the sequence as set forth in SEQ ID NO: 717, and the CDRL3 includes the sequence as set forth in SEQ ID NO: 718, according to Contact.
60. The antibody or fragment thereof of embodiment 59, wherein the CDRH1 is encoded by the sequence as set forth in SEQ ID NO: 1221, the CDRH2 is encoded by the sequence as set forth in SEQ ID NO: 1222, the CDRH3 is encoded by the sequence as set forth in SEQ ID NO: 1223, the CDRL1 is encoded by the sequence as set forth in SEQ ID NO: 1224, the CDRL2 is encoded by the sequence GGTGCATCC, the CDRL3 is encoded by the sequence as set forth in SEQ ID NO: 1225.
61. The antibody or fragment thereof of embodiment 1, wherein Z582-14 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 719, the CDRH2 includes the sequence as set forth in SEQ ID NO: 720, the CDRH3 includes the sequence as set forth in SEQ ID NO: 721, the CDRL1 includes the sequence as set forth in SEQ ID NO: 722, the CDRL2 includes the sequence as set forth in SEQ ID NO: 723, and the CDRL3 includes the sequence as set forth in SEQ ID NO: 724, according to North;
   wherein the CDRH1 includes the sequence as set forth in SEQ ID NO: 725, the CDRH2 includes the sequence as set forth in SEQ ID NO: 726, the CDRH3 includes the sequence as set forth in SEQ ID NO: 727, the CDRL1 includes the sequence as set forth in SEQ ID NO: 722, the CDRL2 includes the sequence as set forth in SEQ ID NO: 728, and the CDRL3 includes the sequence as set forth in SEQ ID NO: 724, according to Kabat;
   wherein the CDRH1 includes the sequence as set forth in SEQ ID NO: 729, the CDRH2 includes the sequence as set forth in SEQ ID NO: 730, the CDRH3 includes the sequence as set forth in SEQ ID NO: 721, the CDRL1 includes the sequence as set forth in SEQ ID NO: 731, the CDRL2 includes the sequence ED, and the CDRL3 includes the sequence as set forth in SEQ ID NO: 724 according to IMGT;
   wherein the CDRH1 includes the sequence as set forth in SEQ ID NO: 732, the CDRH2 includes the sequence as set forth in SEQ ID NO: 733, the CDRH3 includes the sequence as set forth in SEQ ID NO: 727, the CDRL1 includes the sequence as set forth in SEQ ID NO: 722, the CDRL2 includes the sequence as set forth in SEQ ID NO: 728, and the CDRL3 includes the sequence as set forth in SEQ ID NO: 724, according to Chothia; or
   wherein the CDRH1 includes the sequence as set forth in SEQ ID NO: 734, the CDRH2 includes the sequence as set forth in SEQ ID NO: 735, the CDRH3 includes the sequence as set forth in SEQ ID NO: 736, the CDRL1 includes the sequence as set forth in SEQ ID NO: 737, the CDRL2 includes the sequence as set forth in SEQ ID NO: 738, and the CDRL3 includes the sequence as set forth in SEQ ID NO: 739, according to Contact.
62. The antibody or fragment thereof of embodiment 61, wherein the CDRH1 is encoded by the sequence as set forth in SEQ ID NO: 1226, the CDRH2 is encoded by the sequence as set forth in SEQ ID NO: 1227, the CDRH3 is encoded by the sequence as set forth in SEQ ID NO: 1228, the CDRL1 is encoded by the sequence as set forth in SEQ ID NO: 1229, the CDRL2 is encoded by the sequence GAGGATAAC, the CDRL3 is encoded by the sequence as set forth in SEQ ID NO: 1231.
63. The antibody or fragment thereof of embodiment 1, wherein Z582-15 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 719, the CDRH2 includes the sequence as set forth in SEQ ID NO: 720, the CDRH3 includes the sequence as set forth in SEQ ID NO: 721, the CDRL1 includes the sequence as set forth in SEQ ID NO: 740, the CDRL2 includes the sequence as set forth in SEQ ID NO: 741, and the CDRL3 includes the sequence as set forth in SEQ ID NO: 742, according to North;
   wherein the CDRH1 includes the sequence as set forth in SEQ ID NO: 725, the CDRH2 includes the sequence as set forth in SEQ ID NO: 726, the CDRH3 includes the sequence as set forth in SEQ ID NO: 727, the CDRL1 includes the sequence as set forth in SEQ ID NO: 740, the CDRL2 includes the sequence as set forth in SEQ ID NO: 743, and the CDRL3 includes the sequence as set forth in SEQ ID NO: 742, according to Kabat;
   wherein the CDRH1 includes the sequence as set forth in SEQ ID NO: 729, the CDRH2 includes the sequence as set forth in SEQ ID NO: 730, the CDRH3 includes the sequence as set forth in SEQ ID NO: 721, the CDRL1 includes the sequence as set forth in SEQ ID NO: 744, the CDRL2 includes the sequence YD, and the CDRL3 includes the sequence as set forth in SEQ ID NO: 742 according to IMGT;
   wherein the CDRH1 includes the sequence as set forth in SEQ ID NO: 732, the CDRH2 includes the sequence as set forth in SEQ ID NO: 733, the CDRH3 includes the sequence as set forth in SEQ ID NO: 727, the CDRL1 includes the sequence as set forth in SEQ ID NO: 740, the CDRL2 includes the sequence as set forth in SEQ ID NO: 743, and the CDRL3 includes the sequence as set forth in SEQ ID NO: 742, according to Chothia; or
   wherein the CDRH1 includes the sequence as set forth in SEQ ID NO: 734, the CDRH2 includes the sequence as set forth in SEQ ID NO: 735, the CDRH3 includes the sequence as set forth in SEQ ID NO: 736, the CDRL1 includes the sequence as set forth in SEQ ID NO: 745, the CDRL2 includes the sequence as set forth in SEQ ID NO: 746, and the CDRL3 includes the sequence as set forth in SEQ ID NO: 747, according to Contact.
64. The antibody or fragment thereof of embodiment 63, wherein the CDRH1 is encoded by the sequence as set forth in SEQ ID NO: 1226, the CDRH2 is encoded by the sequence as set forth in SEQ ID NO: 1227, the CDRH3 is encoded by the sequence as set forth in SEQ ID NO: 1228, the CDRL1 is encoded by the sequence as set forth in SEQ ID NO: 1232, the CDRL2 is encoded by the sequence TATGATAGC, the CDRL3 is encoded by the sequence as set forth in SEQ ID NO: 1234.
65. The antibody or fragment thereof of embodiment 1, wherein Z082-U1 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 748, the CDRH2 includes the sequence as set forth in SEQ ID NO: 749, the CDRH3 includes the sequence as set forth in SEQ ID NO: 750, the CDRL1 includes the sequence as set forth in SEQ ID NO: 751, the CDRL2 includes the sequence as set forth in SEQ ID NO: 752, and the CDRL3 includes the sequence as set forth in SEQ ID NO: 753, according to North;
   wherein the CDRH1 includes the sequence as set forth in SEQ ID NO: 754, the CDRH2 includes the sequence as set forth in SEQ ID NO: 755, the CDRH3 includes the sequence as set forth in SEQ ID NO: 756, the CDRL1 includes the sequence as set forth in SEQ ID NO: 751, the CDRL2 includes the sequence as set forth in SEQ ID NO: 757, and the CDRL3 includes the sequence as set forth in SEQ ID NO: 753, according to Kabat;
   wherein the CDRH1 includes the sequence as set forth in SEQ ID NO: 758, the CDRH2 includes the sequence as set forth in SEQ ID NO: 759, the CDRH3 includes the sequence as set forth in SEQ ID NO: 750, the CDRL1 includes the sequence as set forth in SEQ ID NO: 760, the CDRL2 includes the sequence DV, and the CDRL3 includes the sequence as set forth in SEQ ID NO: 753 according to IMGT;
   wherein the CDRH1 includes the sequence as set forth in SEQ ID NO: 761, the CDRH2 includes the sequence as set forth in SEQ ID NO: 762, the CDRH3 includes the sequence as set forth in SEQ ID NO: 756, the CDRL1 includes the sequence as set forth in SEQ ID NO: 751, the CDRL2 includes the sequence as set forth in SEQ ID NO: 757, and the CDRL3 includes the sequence as set forth in SEQ ID NO: 753, according to Chothia; or
   wherein the CDRH1 includes the sequence as set forth in SEQ ID NO: 763, the CDRH2 includes the sequence as set forth in SEQ ID NO: 764, the CDRH3 includes the sequence as set forth in SEQ ID NO: 765, the CDRL1 includes the sequence as set forth in SEQ ID NO: 766, the CDRL2 includes the sequence as set forth in SEQ ID NO: 767, and the CDRL3 includes the sequence as set forth in SEQ ID NO: 768, according to Contact.
66. The antibody or fragment thereof of embodiment 65, wherein the CDRH1 is encoded by the sequence as set forth in SEQ ID NO: 1235, the CDRH2 is encoded by the sequence as set forth in SEQ ID NO: 1236, the CDRH3 is encoded by the sequence as set forth in SEQ ID NO: 1237, the CDRL1 is encoded by the sequence as set forth in SEQ ID NO: 1238, the CDRL2 is encoded by the sequence GATGTCACT, the CDRL3 is encoded by the sequence as set forth in SEQ ID NO: 1239.
67. The antibody or fragment thereof of embodiment 1, wherein Z082-U5 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 769, the CDRH2 includes the sequence as set forth in SEQ ID NO: 770, the CDRH3 includes the sequence as set forth in SEQ ID NO: 771, the CDRL1 includes the sequence as set forth in SEQ ID NO: 772, the CDRL2 includes the sequence as set forth in SEQ ID NO: 773, and the CDRL3 includes the sequence as set forth in SEQ ID NO: 774, according to North;
   wherein the CDRH1 includes the sequence as set forth in SEQ ID NO: 775, the CDRH2 includes the sequence as set forth in SEQ ID NO: 776, the CDRH3 includes the sequence as set forth in SEQ ID NO: 777, the CDRL1 includes the sequence as set forth in SEQ ID NO: 772, the CDRL2 includes the sequence as set forth in SEQ ID NO: 778, and the CDRL3 includes the sequence as set forth in SEQ ID NO: 774, according to Kabat;
   wherein the CDRH1 includes the sequence as set forth in SEQ ID NO: 779, the CDRH2 includes the sequence as set forth in SEQ ID NO: 780, the CDRH3 includes the sequence as set forth in SEQ ID NO: 771, the CDRL1 includes the sequence as set forth in SEQ ID NO: 781, the CDRL2 includes the sequence DD, and the CDRL3 includes the sequence as set forth in SEQ ID NO: 774 according to IMGT;
   wherein the CDRH1 includes the sequence as set forth in SEQ ID NO: 782, the CDRH2 includes the sequence as set forth in SEQ ID NO: 783, the CDRH3 includes the sequence as set forth in SEQ ID NO: 777, the CDRL1 includes the sequence as set forth in SEQ ID NO: 772, the CDRL2 includes the sequence as set forth in SEQ ID NO: 778, and the CDRL3 includes the sequence as set forth in SEQ ID NO: 774, according to Chothia; or
   wherein the CDRH1 includes the sequence as set forth in SEQ ID NO: 784, the CDRH2 includes the sequence as set forth in SEQ ID NO: 785, the CDRH3 includes the sequence as set forth in SEQ ID NO: 786, the CDRL1 includes the sequence as set forth in SEQ ID NO: 745, the CDRL2 includes the sequence as set forth in SEQ ID NO: 787, and the CDRL3 includes the sequence as set forth in SEQ ID NO: 788, according to Contact.
68. The antibody or fragment thereof of embodiment 67, wherein the CDRH1 is encoded by the sequence as set forth in SEQ ID NO: 1240, the CDRH2 is encoded by the sequence as set forth in SEQ ID NO: 1241, the CDRH3 is encoded by the sequence as set forth in SEQ ID NO: 1242, the CDRL1 is encoded by the sequence as set forth in SEQ ID NO: 1243, the CDRL2 is encoded by the sequence GATGATAGT, the CDRL3 is encoded by the sequence as set forth in SEQ ID NO: 1245.
69. The antibody or fragment thereof of embodiment 1, wherein Z082-U6 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 789, the CDRH2 includes the sequence as set forth in SEQ ID NO: 790, the CDRH3 includes the sequence as set forth in SEQ ID NO: 791, the CDRL1 includes the sequence as set forth in SEQ ID NO: 792, the CDRL2 includes the sequence as set forth in SEQ ID NO: 793, and the CDRL3 includes the sequence as set forth in SEQ ID NO: 794, according to North;
   wherein the CDRH1 includes the sequence as set forth in SEQ ID NO: 795, the CDRH2 includes the sequence as set forth in SEQ ID NO: 796, the CDRH3 includes the sequence as set forth in SEQ ID NO: 797, the CDRL1 includes the sequence as set forth in SEQ ID NO: 792, the CDRL2 includes the sequence as set forth in SEQ ID NO: 798, and the CDRL3 includes the sequence as set forth in SEQ ID NO: 794, according to Kabat;
   wherein the CDRH1 includes the sequence as set forth in SEQ ID NO: 799, the CDRH2 includes the sequence as set forth in SEQ ID NO: 800, the CDRH3 includes the sequence as set forth in SEQ ID NO: 791, the CDRL1 includes the sequence as set forth in SEQ ID NO: 801, the CDRL2 includes the sequence DA, and the CDRL3 includes the sequence as set forth in SEQ ID NO: 794 according to IMGT;
   wherein the CDRH1 includes the sequence as set forth in SEQ ID NO: 802, the CDRH2 includes the sequence as set forth in SEQ ID NO: 803, the CDRH3 includes the sequence as set forth in SEQ ID NO: 797, the CDRL1 includes the sequence as set forth in SEQ ID NO: 792, the CDRL2 includes the sequence as set forth in SEQ ID NO: 798, and the CDRL3 includes the sequence as set forth in SEQ ID NO: 794, according to Chothia; or
   wherein the CDRH1 includes the sequence as set forth in SEQ ID NO: 804, the CDRH2 includes the sequence as set forth in SEQ ID NO: 805, the CDRH3 includes the sequence as set forth in SEQ ID NO: 806, the CDRL1 includes the sequence as set forth in SEQ ID NO: 807, the CDRL2 includes the sequence as set forth in SEQ ID NO: 808, and the CDRL3 includes the sequence as set forth in SEQ ID NO: 809, according to Contact.
70. The antibody or fragment thereof of embodiment 69, wherein the CDRH1 is encoded by the sequence as set forth in SEQ ID NO: 1246, the CDRH2 is encoded by the sequence as set forth in SEQ ID NO: 1247, the CDRH3 is encoded by the sequence as set forth in SEQ ID NO: 1248, the CDRL1 is encoded by the sequence as set forth in SEQ ID NO: 1249, the CDRL2 is encoded by the sequence GATGCATCC, the CDRL3 is encoded by the sequence as set forth in SEQ ID NO: 1251.
71. The antibody or fragment thereof of embodiment 1, wherein W056-01 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 810, the CDRH2 includes the sequence as set forth in SEQ ID NO: 811, the CDRH3 includes the sequence as set forth in SEQ ID NO: 315, the CDRL1 includes the sequence as set forth in SEQ ID NO: 812, the CDRL2 includes the sequence as set forth in SEQ ID NO: 813, and the CDRL3 includes the sequence as set forth in SEQ ID NO: 814, according to North;
   wherein the CDRH1 includes the sequence as set forth in SEQ ID NO: 319, the CDRH2 includes the sequence as set forth in SEQ ID NO: 815, the CDRH3 includes the sequence as set forth in SEQ ID NO: 321, the CDRL1 includes the sequence as set forth in SEQ ID NO: 812, the CDRL2 includes the sequence as set forth in SEQ ID NO: 816, and the CDRL3 includes the sequence as set forth in SEQ ID NO: 814, according to Kabat;
   wherein the CDRH1 includes the sequence as set forth in SEQ ID NO: 817, the CDRH2 includes the sequence as set forth in SEQ ID NO: 818, the CDRH3 includes the sequence as set forth in SEQ ID NO: 315, the CDRL1 includes the sequence as set forth in SEQ ID NO: 819, the CDRL2 includes the sequence KA, and the CDRL3 includes the sequence as set forth in SEQ ID NO: 814 according to IMGT;
   wherein the CDRH1 includes the sequence as set forth in SEQ ID NO: 820, the CDRH2 includes the sequence as set forth in SEQ ID NO: 821, the CDRH3 includes the sequence as set forth in SEQ ID NO: 321, the CDRL1 includes the sequence as set forth in SEQ ID NO: 812, the CDRL2 includes the sequence as set forth in SEQ ID NO: 816, and the CDRL3 includes the sequence as set forth in SEQ ID NO: 814, according to Chothia; or
   wherein the CDRH1 includes the sequence as set forth in SEQ ID NO: 822, the CDRH2 includes the sequence as set forth in SEQ ID NO: 823, the CDRH3 includes the sequence as set forth in SEQ ID NO: 325, the CDRL1 includes the sequence as set forth in SEQ ID NO: 326, the CDRL2 includes the sequence as set forth in SEQ ID NO: 824, and the CDRL3 includes the sequence as set forth in SEQ ID NO: 825, according to Contact.
72. The antibody or fragment thereof of embodiment 71, wherein the CDRH1 is encoded by the sequence as set forth in SEQ ID NO: 1252, the CDRH2 is encoded by the sequence as set forth in SEQ ID NO: 1253, the CDRH3 is encoded by the sequence as set forth in SEQ ID NO: 1254, the CDRL1 is encoded by the sequence as set forth in SEQ ID NO: 1255, the CDRL2 is encoded by the sequence AAGGCGTCT, the CDRL3 is encoded by the sequence as set forth in SEQ ID NO: 1257.
73. The antibody or fragment thereof of embodiment 1, wherein W056-02 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 826, the CDRH2 includes the sequence as set forth in SEQ ID NO: 827, the CDRH3 includes the sequence as set forth in SEQ ID NO: 828, the CDRL1 includes the sequence as set forth in SEQ ID NO: 829, the CDRL2 includes the sequence as set forth in SEQ ID NO: 830, and the CDRL3 includes the sequence as set forth in SEQ ID NO: 831, according to North;
   wherein the CDRH1 includes the sequence as set forth in SEQ ID NO: 319, the CDRH2 includes the sequence as set forth in SEQ ID NO: 832, the CDRH3 includes the sequence as set forth in SEQ ID NO: 833, the CDRL1 includes the sequence as set forth in SEQ ID NO: 829, the CDRL2 includes the sequence as set forth in SEQ ID NO: 834, and the CDRL3 includes the sequence as set forth in SEQ ID NO: 831, according to Kabat;
   wherein the CDRH1 includes the sequence as set forth in SEQ ID NO: 835, the CDRH2 includes the sequence as set forth in SEQ ID NO: 324, the CDRH3 includes the sequence as set forth in SEQ ID NO: 828, the CDRL1 includes the sequence as set forth in SEQ ID NO: 836, the CDRL2 includes the sequence KV, and the CDRL3 includes the sequence as set forth in SEQ ID NO: 831 according to IMGT;
   wherein the CDRH1 includes the sequence as set forth in SEQ ID NO: 837, the CDRH2 includes the sequence as set forth in SEQ ID NO: 327, the CDRH3 includes the sequence as set forth in SEQ ID NO: 833, the CDRL1 includes the sequence as set forth in SEQ ID NO: 829, the CDRL2 includes the sequence as set forth in SEQ ID NO: 834, and the CDRL3 includes the sequence as set forth in SEQ ID NO: 831, according to Chothia; or
   wherein the CDRH1 includes the sequence as set forth in SEQ ID NO: 822, the CDRH2 includes the sequence as set forth in SEQ ID NO: 838, the CDRH3 includes the sequence as set forth in SEQ ID NO: 839, the CDRL1 includes the sequence as set forth in SEQ ID NO: 840, the CDRL2 includes the sequence as set forth in SEQ ID NO: 841, and the CDRL3 includes the sequence as set forth in SEQ ID NO: 842, according to Contact.
74. The antibody or fragment thereof of embodiment 73, wherein the CDRH1 is encoded by the sequence as set forth in SEQ ID NO: 1258, the CDRH2 is encoded by the sequence as set forth in SEQ ID NO: 1259, the CDRH3 is encoded by the sequence as set forth in SEQ ID NO: 1260, the CDRL1 is encoded by the sequence as set forth in SEQ ID NO: 1261, the CDRL2 is encoded by the sequence AAAGTTTCC, the CDRL3 is encoded by the sequence as set forth in SEQ ID NO: 1263.
75. The antibody or fragment thereof of embodiment 1, wherein W917-01 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 843, the CDRH2 includes the sequence as set forth in SEQ ID NO: 844, the CDRH3 includes the sequence as set forth in SEQ ID NO: 845, the CDRL1 includes the sequence as set forth in SEQ ID NO: 295, the CDRL2 includes the sequence as set forth in SEQ ID NO: 296, and the CDRL3 includes the sequence as set forth in SEQ ID NO: 846, according to North;
   wherein the CDRH1 includes the sequence as set forth in SEQ ID NO: 847, the CDRH2 includes the sequence as set forth in SEQ ID NO: 848, the CDRH3 includes the sequence as set forth in SEQ ID NO: 849, the CDRL1 includes the sequence as set forth in SEQ ID NO: 295, the CDRL2 includes the sequence as set forth in SEQ ID NO: 301, and the CDRL3 includes the sequence as set forth in SEQ ID NO: 846, according to Kabat;
   wherein the CDRH1 includes the sequence as set forth in SEQ ID NO: 850, the CDRH2 includes the sequence as set forth in SEQ ID NO: 851, the CDRH3 includes the sequence as set forth in SEQ ID NO: 845, the CDRL1 includes the sequence as set forth in SEQ ID NO: 304, the CDRL2 includes the sequence AA, and the CDRL3 includes the sequence as set forth in SEQ ID NO: 846 according to IMGT;
   wherein the CDRH1 includes the sequence as set forth in SEQ ID NO: 852, the CDRH2 includes the sequence as set forth in SEQ ID NO: 853, the CDRH3 includes the sequence as set forth in SEQ ID NO: 849, the CDRL1 includes the sequence as set forth in SEQ ID NO: 295, the CDRL2 includes the sequence as set forth in SEQ ID NO: 301, and the CDRL3 includes the sequence as set forth in SEQ ID NO: 846, according to Chothia; or
   wherein the CDRH1 includes the sequence as set forth in SEQ ID NO: 854, the CDRH2 includes the sequence as set forth in SEQ ID NO: 855, the CDRH3 includes the sequence as set forth in SEQ ID NO: 856, the CDRL1 includes the sequence as set forth in SEQ ID NO: 310, the CDRL2 includes the sequence as set forth in SEQ ID NO: 857, and the CDRL3 includes the sequence as set forth in SEQ ID NO: 846, according to Contact.
76. The antibody or fragment thereof of embodiment 75, wherein the CDRH1 is encoded by the sequence as set forth in SEQ ID NO: 1264, the CDRH2 is encoded by the sequence as set forth in SEQ ID NO: 1265, the CDRH3 is encoded by the sequence as set forth in SEQ ID NO: 1266, the CDRL1 is encoded by the sequence as set forth in SEQ ID NO: 1267, the CDRL2 is encoded by the sequence GCTGCATCC, the CDRL3 is encoded by the sequence as set forth in SEQ ID NO: 1268.
77. The antibody or fragment thereof of embodiment 1, wherein W917-02 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 858, the CDRH2 includes the sequence as set forth in SEQ ID NO: 859, the CDRH3 includes the sequence as set forth in SEQ ID NO: 860, the CDRL1 includes the sequence as set forth in SEQ ID NO: 861, the CDRL2 includes the sequence as set forth in SEQ ID NO: 862, and the CDRL3 includes the sequence as set forth in SEQ ID NO: 863, according to North;
   wherein the CDRH1 includes the sequence as set forth in SEQ ID NO: 864, the CDRH2 includes the sequence as set forth in SEQ ID NO: 865, the CDRH3 includes the sequence as set forth in SEQ ID NO: 866, the CDRL1 includes the sequence as set forth in SEQ ID NO: 861, the CDRL2 includes the sequence as set forth in SEQ ID NO: 867, and the CDRL3 includes the sequence as set forth in SEQ ID NO: 863, according to Kabat;
   wherein the CDRH1 includes the sequence as set forth in SEQ ID NO: 864, the CDRH2 includes the sequence as set forth in SEQ ID NO: 865, the CDRH3 includes the sequence as set forth in SEQ ID NO: 860, the CDRL1 includes the sequence as set forth in SEQ ID NO: 866, the CDRL2 includes the sequence KA, and the CDRL3 includes the sequence as set forth in SEQ ID NO: 863 according to IMGT;
   wherein the CDRH1 includes the sequence as set forth in SEQ ID NO: 305, the CDRH2 includes the sequence as set forth in SEQ ID NO: 867, the CDRH3 includes the sequence as set forth in SEQ ID NO: 866, the CDRL1 includes the sequence as set forth in SEQ ID NO: 861, the CDRL2 includes the sequence as set forth in SEQ ID NO: 867, and the CDRL3 includes the sequence as set forth in SEQ ID NO: 863, according to Chothia; or
   wherein the CDRH1 includes the sequence as set forth in SEQ ID NO: 868, the CDRH2 includes the sequence as set forth in SEQ ID NO: 869, the CDRH3 includes the sequence as set forth in SEQ ID NO: 870, the CDRL1 includes the sequence as set forth in SEQ ID NO: 871, the CDRL2 includes the sequence as set forth in SEQ ID NO: 872, and the CDRL3 includes the sequence as set forth in SEQ ID NO: 863, according to Contact.
78. The antibody or fragment thereof of embodiment 77, wherein the CDRH1 is encoded by the sequence as set forth in SEQ ID NO: 1269, the CDRH2 is encoded by the sequence as set forth in SEQ ID NO: 1270, the CDRH3 is encoded by the sequence as set forth in SEQ ID NO: 1271, the CDRL1 is encoded by the sequence as set forth in SEQ ID NO: 1272, the CDRL2 is encoded by the sequence AAGGCGTCT, the CDRL3 is encoded by the sequence as set forth in SEQ ID NO: 1273.
79. The antibody or fragment thereof of embodiment 1, wherein W917-03 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 873, the CDRH2 includes the sequence as set forth in SEQ ID NO: 874, the CDRH3 includes the sequence as set forth in SEQ ID NO: 875, the CDRL1 includes the sequence as set forth in SEQ ID NO: 360, the CDRL2 includes the sequence as set forth in SEQ ID NO: 361, and the CDRL3 includes the sequence as set forth in SEQ ID NO: 876, according to North;
   wherein the CDRH1 includes the sequence as set forth in SEQ ID NO: 877, the CDRH2 includes the sequence as set forth in SEQ ID NO: 878, the CDRH3 includes the sequence as set forth in SEQ ID NO: 879, the CDRL1 includes the sequence as set forth in SEQ ID NO: 360, the CDRL2 includes the sequence as set forth in SEQ ID NO: 366, and the CDRL3 includes the sequence as set forth in SEQ ID NO: 876, according to Kabat;
   wherein the CDRH1 includes the sequence as set forth in SEQ ID NO: 880, the CDRH2 includes the sequence as set forth in SEQ ID NO: 881, the CDRH3 includes the sequence as set forth in SEQ ID NO: 875, the CDRL1 includes the sequence as set forth in SEQ ID NO: 369, the CDRL2 includes the sequence KD, and the CDRL3 includes the sequence as set forth in SEQ ID NO: 876 according to IMGT;
   wherein the CDRH1 includes the sequence as set forth in SEQ ID NO: 882, the CDRH2 includes the sequence as set forth in SEQ ID NO: 883, the CDRH3 includes the sequence as set forth in SEQ ID NO: 879, the CDRL1 includes the sequence as set forth in SEQ ID NO: 360, the CDRL2 includes the sequence as set forth in SEQ ID NO: 366, and the CDRL3 includes the sequence as set forth in SEQ ID NO: 876, according to Chothia; or
   wherein the CDRH1 includes the sequence as set forth in SEQ ID NO: 884, the CDRH2 includes the sequence as set forth in SEQ ID NO: 885, the CDRH3 includes the sequence as set forth in SEQ ID NO: 886, the CDRL1 includes the sequence as set forth in SEQ ID NO: 74, the CDRL2 includes the sequence as set forth in SEQ ID NO: 375, and the CDRL3 includes the sequence as set forth in SEQ ID NO: 887, according to Contact.
80. The antibody or fragment thereof of embodiment 79, wherein the CDRH1 is encoded by the sequence as set forth in SEQ ID NO: 1274, the CDRH2 is encoded by the sequence as set forth in SEQ ID NO: 1275, the CDRH3 is encoded by the sequence as set forth in SEQ ID NO: 1276, the CDRL1 is encoded by the sequence as set forth in SEQ ID NO: 1127, the CDRL2 is encoded by the sequence AAAGACAGT, the CDRL3 is encoded by the sequence as set forth in SEQ ID NO: 1277.
81. The antibody or fragment thereof of embodiment 1, wherein Z504-01 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 888, the CDRH2 includes the sequence as set forth in SEQ ID NO: 889, the CDRH3 includes the sequence as set forth in SEQ ID NO: 890, the CDRL1 includes the sequence as set forth in SEQ ID NO: 891, the CDRL2 includes the sequence as set forth in SEQ ID NO: 741, and the CDRL3 includes the sequence as set forth in SEQ ID NO: 892, according to North;
   wherein the CDRH1 includes the sequence as set forth in SEQ ID NO: 893, the CDRH2 includes the sequence as set forth in SEQ ID NO: 894, the CDRH3 includes the sequence as set forth in SEQ ID NO: 895, the CDRL1 includes the sequence as set forth in SEQ ID NO: 891, the CDRL2 includes the sequence as set forth in SEQ ID NO: 743, and the CDRL3 includes the sequence as set forth in SEQ ID NO: 892, according to Kabat;
   wherein the CDRH1 includes the sequence as set forth in SEQ ID NO: 896, the CDRH2 includes the sequence as set forth in SEQ ID NO: 897, the CDRH3 includes the sequence as set forth in SEQ ID NO: 890, the CDRL1 includes the sequence as set forth in SEQ ID NO: 898, the CDRL2 includes the sequence YD, and the CDRL3 includes the sequence as set forth in SEQ ID NO: 892 according to IMGT;
   wherein the CDRH1 includes the sequence as set forth in SEQ ID NO: 899, the CDRH2 includes the sequence as set forth in SEQ ID NO: 900, the CDRH3 includes the sequence as set forth in SEQ ID NO: 895, the CDRL1 includes the sequence as set forth in SEQ ID NO: 891, the CDRL2 includes the sequence as set forth in SEQ ID NO: 743, and the CDRL3 includes the sequence as set forth in SEQ ID NO: 892, according to Chothia; or
   wherein the CDRH1 includes the sequence as set forth in SEQ ID NO: 901, the CDRH2 includes the sequence as set forth in SEQ ID NO: 902, the CDRH3 includes the sequence as set forth in SEQ ID NO: 903, the CDRL1 includes the sequence as set forth in SEQ ID NO: 745, the CDRL2 includes the sequence as set forth in SEQ ID NO: 904, and the CDRL3 includes the sequence as set forth in SEQ ID NO: 905, according to Contact.
82. The antibody or fragment thereof of embodiment 81, wherein the CDRH1 is encoded by the sequence as set forth in SEQ ID NO: 1278, the CDRH2 is encoded by the sequence as set forth in SEQ ID NO: 1279, the CDRH3 is encoded by the sequence as set forth in SEQ ID NO: 1280, the CDRL1 is encoded by the sequence as set forth in SEQ ID NO: 1281, the CDRL2 is encoded by the sequence TATGATAGC, the CDRL3 is encoded by the sequence as set forth in SEQ ID NO: 1282.
83. The antibody or fragment thereof of embodiment 1, wherein Z504-02 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 906, the CDRH2 includes the sequence as set forth in SEQ ID NO: 907, the CDRH3 includes the sequence as set forth in SEQ ID NO: 908, the CDRL1 includes the sequence as set forth in SEQ ID NO: 861, the CDRL2 includes the sequence as set forth in SEQ ID NO: 909, and the CDRL3 includes the sequence as set forth in SEQ ID NO: 910, according to North;
   wherein the CDRH1 includes the sequence as set forth in SEQ ID NO: 911, the CDRH2 includes the sequence as set forth in SEQ ID NO: 912, the CDRH3 includes the sequence as set forth in SEQ ID NO: 913, the CDRL1 includes the sequence as set forth in SEQ ID NO: 861, the CDRL2 includes the sequence as set forth in SEQ ID NO: 914, and the CDRL3 includes the sequence as set forth in SEQ ID NO: 910, according to Kabat;
   wherein the CDRH1 includes the sequence as set forth in SEQ ID NO: 915, the CDRH2 includes the sequence as set forth in SEQ ID NO: 916, the CDRH3 includes the sequence as set forth in SEQ ID NO: 908, the CDRL1 includes the sequence as set forth in SEQ ID NO: 866, the CDRL2 includes the sequence KA, and the CDRL3 includes the sequence as set forth in SEQ ID NO: 910 according to IMGT;
   wherein the CDRH1 includes the sequence as set forth in SEQ ID NO: 917, the CDRH2 includes the sequence as set forth in SEQ ID NO: 918, the CDRH3 includes the sequence as set forth in SEQ ID NO: 913, the CDRL1 includes the sequence as set forth in SEQ ID NO: 861, the CDRL2 includes the sequence as set forth in SEQ ID NO: 914, and the CDRL3 includes the sequence as set forth in SEQ ID NO: 910, according to Chothia; or
   wherein the CDRH1 includes the sequence as set forth in SEQ ID NO: 919, the CDRH2 includes the sequence as set forth in SEQ ID NO: 920, the CDRH3 includes the sequence as set forth in SEQ ID NO: 921, the CDRL1 includes the sequence as set forth in SEQ ID NO: 871, the CDRL2 includes the sequence as set forth in SEQ ID NO: 922, and the CDRL3 includes the sequence as set forth in SEQ ID NO: 923, according to Contact.
84. The antibody or fragment thereof of embodiment 83, wherein the CDRH1 is encoded by the sequence as set forth in SEQ ID NO: 1283, the CDRH2 is encoded by the sequence as set forth in SEQ ID NO: 1284, the CDRH3 is encoded by the sequence as set forth in SEQ ID NO: 1285, the CDRL1 is encoded by the sequence as set forth in SEQ ID NO: 1272, the CDRL2 is encoded by the sequence AAGGCGTCT, the CDRL3 is encoded by the sequence as set forth in SEQ ID NO: 1286.
85. The antibody or fragment thereof of embodiment 1, wherein Z538-01 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 924, the CDRH2 includes the sequence as set forth in SEQ ID NO: 925, the CDRH3 includes the sequence as set forth in SEQ ID NO: 926, the CDRL1 includes the sequence as set forth in SEQ ID NO: 927, the CDRL2 includes the sequence as set forth in SEQ ID NO: 642, and the CDRL3 includes the sequence as set forth in SEQ ID NO: 928, according to North;
   wherein the CDRH1 includes the sequence as set forth in SEQ ID NO: 929, the CDRH2 includes the sequence as set forth in SEQ ID NO: 930, the CDRH3 includes the sequence as set forth in SEQ ID NO: 931, the CDRL1 includes the sequence as set forth in SEQ ID NO: 927, the CDRL2 includes the sequence as set forth in SEQ ID NO: 647, and the CDRL3 includes the sequence as set forth in SEQ ID NO: 928, according to Kabat;
   wherein the CDRH1 includes the sequence as set forth in SEQ ID NO: 932, the CDRH2 includes the sequence as set forth in SEQ ID NO: 933, the CDRH3 includes the sequence as set forth in SEQ ID NO: 934, the CDRL1 includes the sequence as set forth in SEQ ID NO: 935, the CDRL2 includes the sequence AA, and the CDRL3 includes the sequence as set forth in SEQ ID NO: 928 according to IMGT;
   wherein the CDRH1 includes the sequence as set forth in SEQ ID NO: 936, the CDRH2 includes the sequence as set forth in SEQ ID NO: 937, the CDRH3 includes the sequence as set forth in SEQ ID NO: 931, the CDRL1 includes the sequence as set forth in SEQ ID NO: 927, the CDRL2 includes the sequence as set forth in SEQ ID NO: 647, and the CDRL3 includes the sequence as set forth in SEQ ID NO: 928, according to Chothia; or
   wherein the CDRH1 includes the sequence as set forth in SEQ ID NO: 938, the CDRH2 includes the sequence as set forth in SEQ ID NO: 939, the CDRH3 includes the sequence as set forth in SEQ ID NO: 940, the CDRL1 includes the sequence as set forth in SEQ ID NO: 941, the CDRL2 includes the sequence as set forth in SEQ ID NO: 942, and the CDRL3 includes the sequence as set forth in SEQ ID NO: 943, according to Contact.
86. The antibody or fragment thereof of embodiment 85, wherein the CDRH1 is encoded by the sequence as set forth in SEQ ID NO: 1287, the CDRH2 is encoded by the sequence as set forth in SEQ ID NO: 1288, the CDRH3 is encoded by the sequence as set forth in SEQ ID NO: 1289, the CDRL1 is encoded by the sequence as set forth in SEQ ID NO: 1290, the CDRL2 is encoded by the sequence GCTGCATCC, the CDRL3 is encoded by the sequence as set forth in SEQ ID NO: 1291.
87. The antibody or fragment thereof of embodiment 1, wherein Z538-02 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 944, the CDRH2 includes the sequence as set forth in SEQ ID NO: 945, the CDRH3 includes the sequence as set forth in SEQ ID NO: 946, the CDRL1 includes the sequence as set forth in SEQ ID NO: 947, the CDRL2 includes the sequence as set forth in SEQ ID NO: 948, and the CDRL3 includes the sequence as set forth in SEQ ID NO: 949, according to North;
   wherein the CDRH1 includes the sequence as set forth in SEQ ID NO: 950, the CDRH2 includes the sequence as set forth in SEQ ID NO: 951, the CDRH3 includes the sequence as set forth in SEQ ID NO: 952, the CDRL1 includes the sequence as set forth in SEQ ID NO: 947, the CDRL2 includes the sequence as set forth in SEQ ID NO: 953, and the CDRL3 includes the sequence as set forth in SEQ ID NO: 949, according to Kabat;
   wherein the CDRH1 includes the sequence as set forth in SEQ ID NO: 954, the CDRH2 includes the sequence as set forth in SEQ ID NO: 955, the CDRH3 includes the sequence as set forth in SEQ ID NO: 946, the CDRL1 includes the sequence as set forth in SEQ ID NO: 956, the CDRL2 includes the sequence DV, and the CDRL3 includes the sequence as set forth in SEQ ID NO: 949 according to IMGT;
   wherein the CDRH1 includes the sequence as set forth in SEQ ID NO: 957, the CDRH2 includes the sequence as set forth in SEQ ID NO: 958, the CDRH3 includes the sequence as set forth in SEQ ID NO: 952, the CDRL1 includes the sequence as set forth in SEQ ID NO: 947, the CDRL2 includes the sequence as set forth in SEQ ID NO: 953, and the CDRL3 includes the sequence as set forth in SEQ ID NO: 949, according to Chothia; or
   wherein the CDRH1 includes the sequence as set forth in SEQ ID NO: 959, the CDRH2 includes the sequence as set forth in SEQ ID NO: 960, the CDRH3 includes the sequence as set forth in SEQ ID NO: 961, the CDRL1 includes the sequence as set forth in SEQ ID NO: 962, the CDRL2 includes the sequence as set forth in SEQ ID NO: 963, and the CDRL3 includes the sequence as set forth in SEQ ID NO: 949, according to Contact.
88. The antibody or fragment thereof of embodiment 87, wherein the CDRH1 is encoded by the sequence as set forth in SEQ ID NO: 1292, the CDRH2 is encoded by the sequence as set forth in SEQ ID NO: 1293, the CDRH3 is encoded by the sequence as set forth in SEQ ID NO: 1294, the CDRL1 is encoded by the sequence as set forth in SEQ ID NO: 1295, the CDRL2 is encoded by the sequence GATGTCTCT, the CDRL3 is encoded by the sequence as set forth in SEQ ID NO: 1297.
89. The antibody or fragment thereof of embodiment 1, wherein Z573-01 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 964, the CDRH2 includes the sequence as set forth in SEQ ID NO: 827, the CDRH3 includes the sequence as set forth in SEQ ID NO: 965, the CDRL1 includes the sequence as set forth in SEQ ID NO: 966, the CDRL2 includes the sequence as set forth in SEQ ID NO: 967, and the CDRL3 includes the sequence as set forth in SEQ ID NO: 968, according to North;
   wherein the CDRH1 includes the sequence as set forth in SEQ ID NO: 969, the CDRH2 includes the sequence as set forth in SEQ ID NO: 832, the CDRH3 includes the sequence as set forth in SEQ ID NO: 970, the CDRL1 includes the sequence as set forth in SEQ ID NO: 966, the CDRL2 includes the sequence as set forth in SEQ ID NO: 971, and the CDRL3 includes the sequence as set forth in SEQ ID NO: 968, according to Kabat;
   wherein the CDRH1 includes the sequence as set forth in SEQ ID NO: 972, the CDRH2 includes the sequence as set forth in SEQ ID NO: 324, the CDRH3 includes the sequence as set forth in SEQ ID NO: 965, the CDRL1 includes the sequence as set forth in SEQ ID NO: 973, the CDRL2 includes the sequence WA, and the CDRL3 includes the sequence as set forth in SEQ ID NO: 968 according to IMGT;
   wherein the CDRH1 includes the sequence as set forth in SEQ ID NO: 974, the CDRH2 includes the sequence as set forth in SEQ ID NO: 327, the CDRH3 includes the sequence as set forth in SEQ ID NO: 970, the CDRL1 includes the sequence as set forth in SEQ ID NO: 966, the CDRL2 includes the sequence as set forth in SEQ ID NO: 971, and the CDRL3 includes the sequence as set forth in SEQ ID NO: 968, according to Chothia; or
   wherein the CDRH1 includes the sequence as set forth in SEQ ID NO: 975, the CDRH2 includes the sequence as set forth in SEQ ID NO: 838, the CDRH3 includes the sequence as set forth in SEQ ID NO: 976, the CDRL1 includes the sequence as set forth in SEQ ID NO: 977, the CDRL2 includes the sequence as set forth in SEQ ID NO: 978, and the CDRL3 includes the sequence as set forth in SEQ ID NO: 979, according to Contact.
90. The antibody or fragment thereof of embodiment 89, wherein the CDRH1 is encoded by the sequence as set forth in SEQ ID NO: 1298, the CDRH2 is encoded by the sequence as set forth in SEQ ID NO: 1299, the CDRH3 is encoded by the sequence as set forth in SEQ ID NO: 1300, the CDRL1 is encoded by the sequence as set forth in SEQ ID NO: 1301, the CDRL2 is encoded by the sequence TGGGCTTCT, the CDRL3 is encoded by the sequence as set forth in SEQ ID NO: 1303.
91. The antibody or fragment thereof of embodiment 1, wherein Z573-02 includes a heavy chain variable region including a complementarity determining region (CDR) heavy (H)1, CDRH2, and CDRH3 and a light chain variable region including a CDR light (L)1, CDRL2, and CDRL3;
   wherein the CDRH1 includes the sequence as set forth in SEQ ID NO: 980, the CDRH2 includes the sequence as set forth in SEQ ID NO: 981, the CDRH3 includes the sequence as set forth in SEQ ID NO: 683, the CDRL1 includes the sequence as set forth in SEQ ID NO: 68, the CDRL2 includes the sequence as set forth in SEQ ID NO: 361, and the CDRL3 includes the sequence as set forth in SEQ ID NO: 982, according to North;
   wherein the CDRH1 includes the sequence as set forth in SEQ ID NO: 983, the CDRH2 includes the sequence as set forth in SEQ ID NO: 984, the CDRH3 includes the sequence as set forth in SEQ ID NO: 688, the CDRL1 includes the sequence as set forth in SEQ ID NO: 684, the CDRL2 includes the sequence as set forth in SEQ ID NO: 366, and the CDRL3 includes the sequence as set forth in SEQ ID NO: 982, according to Kabat;
   wherein the CDRH1 includes the sequence as set forth in SEQ ID NO: 985, the CDRH2 includes the sequence as set forth in SEQ ID NO: 986, the CDRH3 includes the sequence as set forth in SEQ ID NO: 683, the CDRL1 includes the sequence as set forth in SEQ ID NO: 691, the CDRL2 includes the sequence KD, and the CDRL3 includes the sequence as set forth in SEQ ID NO: 982 according to IMGT;
   wherein the CDRH1 includes the sequence as set forth in SEQ ID NO: 987, the CDRH2 includes the sequence as set forth in SEQ ID NO: 988, the CDRH3 includes the sequence as set forth in SEQ ID NO: 688, the CDRL1 includes the sequence as set forth in SEQ ID NO: 684, the CDRL2 includes the sequence as set forth in SEQ ID NO: 366, and the CDRL3 includes the sequence as set forth in SEQ ID NO: 982, according to Chothia; or
   wherein the CDRH1 includes the sequence as set forth in SEQ ID NO: 989, the CDRH2 includes the sequence as set forth in SEQ ID NO: 990, the CDRH3 includes the sequence as set forth in SEQ ID NO: 696, the CDRL1 includes the sequence as set forth in SEQ ID NO: 697, the CDRL2 includes the sequence as set forth in SEQ ID NO: 375, and the CDRL3 includes the sequence as set forth in SEQ ID NO: 982, according to Contact.
92. The antibody or fragment thereof of embodiment 91, wherein the CDRH1 is encoded by the sequence as set forth in SEQ ID NO: 1304, the CDRH2 is encoded by the sequence as set forth in SEQ ID NO: 1305, the CDRH3 is encoded by the sequence as set forth in SEQ ID NO: 1306, the CDRL1 is encoded by the sequence as set forth in SEQ ID NO: 1219, the CDRL2 is encoded by the sequence AAAGACAGT, the CDRL3 is encoded by the sequence as set forth in SEQ ID NO: 1307.
93. The antibody or fragment thereof of embodiment 2, wherein the heavy chain variable region includes the sequence as set forth in SEQ ID NO: 47 and the light chain variable region includes the sequence as set forth in SEQ ID NO: 48; or the heavy chain variable region includes a sequence having at least 90% sequence identity to the sequence as set forth in SEQ ID NO: 47 and the light chain variable region includes a sequence having at least 90% sequence identity to the sequence as set forth in SEQ ID NO: 48.
94. The antibody or fragment thereof of embodiment 3, wherein the heavy chain variable region includes a sequence as set forth in SEQ ID NO: 49 and the light chain variable region includes the sequence as set forth in SEQ ID NO: 50; or the heavy chain variable region includes a sequence having at least 90% sequence identity to the sequence as set forth in SEQ ID NO: 49 and the light chain variable region includes a sequence having at least 90% sequence identity to the sequence as set forth in SEQ ID NO: 50.
95. The antibody or fragment thereof of embodiment 4, wherein the heavy chain variable region includes the sequence as set forth in SEQ ID NO: 51 and the light chain variable region includes the sequence as set forth in SEQ ID NO: 52; or the heavy chain variable region includes a sequence having at least 90% sequence identity to the sequence as set forth in SEQ ID NO: 51 and the light chain variable region includes a sequence having at least 90% sequence identity to the sequence as set forth in SEQ ID NO: 52.
96. The antibody or fragment thereof of embodiment 5, wherein the heavy chain variable region includes a sequence as set forth in SEQ ID NO: 53 and the light chain variable region includes the sequence as set forth in SEQ ID NO: 54; or the heavy chain variable region includes a sequence having at least 90% sequence identity to the sequence as set forth in SEQ ID NO: 53 and the light chain variable region includes a sequence having at least 90% sequence identity to the sequence as set forth in SEQ ID NO: 54.
97. The antibody or fragment thereof of embodiment 6, wherein the heavy chain variable region includes a sequence as set forth in SEQ ID NO: 55 and the light chain variable region includes the sequence as set forth in SEQ ID NO: 56; or the heavy chain variable region includes a sequence having at least 90% sequence identity to the sequence as set forth in SEQ ID NO: 55 and the light chain variable region includes a sequence having at least 90% sequence identity to the sequence as set forth in SEQ ID NO: 56.
98. The antibody or fragment thereof of embodiment 7, wherein the heavy chain variable region includes a sequence as set forth in SEQ ID NO: 169 and the light chain variable region includes the sequence as set forth in SEQ ID NO: 170; or the heavy chain variable region includes a sequence having at least 90% sequence identity to the sequence as set forth in SEQ ID NO: 169 and the light chain variable region includes a sequence having at least 90% sequence identity to the sequence as set forth in SEQ ID NO: 170.
99. The antibody or fragment thereof of embodiment 9, wherein the heavy chain variable region includes a sequence as set forth in SEQ ID NO: 171 and the light chain variable region includes the sequence as set forth in SEQ ID NO: 172; or the heavy chain variable region includes a sequence having at least 90% sequence identity to the sequence as set forth in SEQ ID NO: 171 and the light chain variable region includes a sequence having at least 90% sequence identity to the sequence as set forth in SEQ ID NO: 172.
100. The antibody or fragment thereof of embodiment 11, wherein the heavy chain variable region includes a sequence as set forth in SEQ ID NO: 173 and the light chain variable region includes the sequence as set forth in SEQ ID NO: 174; or the heavy chain variable region includes a sequence having at least 90% sequence identity to the sequence as set forth in SEQ ID NO: 173 and the light chain variable region includes a sequence having at least 90% sequence identity to the sequence as set forth in SEQ ID NO: 174.
101. The antibody or fragment thereof of embodiment 13, wherein the heavy chain variable region includes a sequence as set forth in SEQ ID NO: 175 and the light chain variable region includes the sequence as set forth in SEQ ID NO: 176; or the heavy chain variable region includes a sequence having at least 90% sequence identity to the sequence as set forth in SEQ ID NO: 175 and the light chain variable region includes a sequence having at least 90% sequence identity to the sequence as set forth in SEQ ID NO: 176.
102. The antibody or fragment thereof of embodiment 15, wherein the heavy chain variable region includes a sequence as set forth in SEQ ID NO: 177 and the light chain variable region includes the sequence as set forth in SEQ ID NO: 178; or the heavy chain variable region includes a sequence having at least 90% sequence identity to the sequence as set forth in SEQ ID NO: 177 and the light chain variable region includes a sequence having at least 90% sequence identity to the sequence as set forth in SEQ ID NO: 178.
103. The antibody or fragment thereof of embodiment 17, wherein the heavy chain variable region includes a sequence as set forth in SEQ ID NO: 179 and the light chain variable region includes the sequence as set forth in SEQ ID NO: 180; or the heavy chain variable region includes a sequence having at least 90% sequence identity to the sequence as set forth in SEQ ID NO: 179 and the light chain variable region includes a sequence having at least 90% sequence identity to the sequence as set forth in SEQ ID NO: 180.
104. The antibody or fragment thereof of embodiment 19, wherein the heavy chain variable region includes a sequence as set forth in SEQ ID NO: 181 and the light chain variable region includes the sequence as set forth in SEQ ID NO: 182; or the heavy chain variable region includes a sequence having at least 90% sequence identity to the sequence as set forth in SEQ ID NO: 181 and the light chain variable region includes a sequence having at least 90% sequence identity to the sequence as set forth in SEQ ID NO: 182.
105. The antibody or fragment thereof of embodiment 21, wherein the heavy chain variable region includes a sequence as set forth in SEQ ID NO: 183 and the light chain variable region includes the sequence as set forth in SEQ ID NO: 184; or the heavy chain variable region includes a sequence having at least 90% sequence identity to the sequence as set forth in SEQ ID NO: 183 and the light chain variable region includes a sequence having at least 90% sequence identity to the sequence as set forth in SEQ ID NO: 184.
106. The antibody or fragment thereof of embodiment 23, wherein the heavy chain variable region includes a sequence as set forth in SEQ ID NO: 185 and the light chain variable region includes the sequence as set forth in SEQ ID NO: 186; or the heavy chain variable region includes a sequence having at least 90% sequence identity to the sequence as set forth in SEQ ID NO: 185 and the light chain variable region includes a sequence having at least 90% sequence identity to the sequence as set forth in SEQ ID NO: 186.
107. The antibody or fragment thereof of embodiment 25, wherein the heavy chain variable region includes a sequence as set forth in SEQ ID NO: 187 and the light chain variable region includes the sequence as set forth in SEQ ID NO: 188; or the heavy chain variable region includes a sequence having at least 90% sequence identity to the sequence as set forth in SEQ ID NO: 187 and the light chain variable region includes a sequence having at least 90% sequence identity to the sequence as set forth in SEQ ID NO: 188.
108. The antibody or fragment thereof of embodiment 27, wherein the heavy chain variable region includes a sequence as set forth in SEQ ID NO: 189 and the light chain variable region includes the sequence as set forth in SEQ ID NO: 190; or the heavy chain variable region includes a sequence having at least 90% sequence identity to the sequence as set forth in SEQ ID NO: 189 and the light chain variable region includes a sequence having at least 90% sequence identity to the sequence as set forth in SEQ ID NO: 190.
109. The antibody or fragment thereof of embodiment 29, wherein the heavy chain variable region includes a sequence as set forth in SEQ ID NO: 191 and the light chain variable region includes the sequence as set forth in SEQ ID NO: 192; or the heavy chain variable region includes a sequence having at least 90% sequence identity to the sequence as set forth in SEQ ID NO: 191 and the light chain variable region includes a sequence having at least 90% sequence identity to the sequence as set forth in SEQ ID NO: 192.
110. The antibody or fragment thereof of embodiment 31, wherein the heavy chain variable region includes a sequence as set forth in SEQ ID NO: 193 and the light chain variable region includes the sequence as set forth in SEQ ID NO: 194; or the heavy chain variable region includes a sequence having at least 90% sequence identity to the sequence as set forth in SEQ ID NO: 193 and the light chain variable region includes a sequence having at least 90% sequence identity to the sequence as set forth in SEQ ID NO: 194.
111. The antibody or fragment thereof of embodiment 33, wherein the heavy chain variable region includes a sequence as set forth in SEQ ID NO: 195 and the light chain variable region includes the sequence as set forth in SEQ ID NO: 196; or the heavy chain variable region includes a sequence having at least 90% sequence identity to the sequence as set forth in SEQ ID NO: 195 and the light chain variable region includes a sequence having at least 90% sequence identity to the sequence as set forth in SEQ ID NO: 196.
112. The antibody or fragment thereof of embodiment 35, wherein the heavy chain variable region includes a sequence as set forth in SEQ ID NO: 197 and the light chain variable region includes the sequence as set forth in SEQ ID NO: 198; or the heavy chain variable region includes a sequence having at least 90% sequence identity to the sequence as set forth in SEQ ID NO: 197 and the light chain variable region includes a sequence having at least 90% sequence identity to the sequence as set forth in SEQ ID NO: 198.
113. The antibody or fragment thereof of embodiment 37, wherein the heavy chain variable region includes a sequence as set forth in SEQ ID NO: 199 and the light chain variable region includes the sequence as set forth in SEQ ID NO: 200; or the heavy chain variable region includes a sequence having at least 90% sequence identity to the sequence as set forth in SEQ ID NO: 199 and the light chain variable region includes a sequence having at least 90% sequence identity to the sequence as set forth in SEQ ID NO: 200.
114. The antibody or fragment thereof of embodiment 39, wherein the heavy chain variable region includes a sequence as set forth in SEQ ID NO: 1309 and the light chain variable region includes the sequence as set forth in SEQ ID NO: 1310; or the heavy chain variable region includes a sequence having at least 90% sequence identity to the sequence as set forth in SEQ ID NO: 1309 and the light chain variable region includes a sequence having at least 90% sequence identity to the sequence as set forth in SEQ ID NO: 1310.
115. The antibody or fragment thereof of embodiment 41, wherein the heavy chain variable region includes a sequence as set forth in SEQ ID NO: 201 and the light chain variable region includes the sequence as set forth in SEQ ID NO: 202; or the heavy chain variable region includes a sequence having at least 90% sequence identity to the sequence as set forth in SEQ ID NO: 201 and the light chain variable region includes a sequence having at least 90% sequence identity to the sequence as set forth in SEQ ID NO: 202.
116. The antibody or fragment thereof of embodiment 43, wherein the heavy chain variable region includes a sequence as set forth in SEQ ID NO: 203 and the light chain variable region includes the sequence as set forth in SEQ ID NO: 204; or the heavy chain variable region includes a sequence having at least 90% sequence identity to the sequence as set forth in SEQ ID NO: 203 and the light chain variable region includes a sequence having at least 90% sequence identity to the sequence as set forth in SEQ ID NO: 204.
117. The antibody or fragment thereof of embodiment 45, wherein the heavy chain variable region includes a sequence as set forth in SEQ ID NO: 205 and the light chain variable region includes the sequence as set forth in SEQ ID NO: 206; or the heavy chain variable region includes a sequence having at least 90% sequence identity to the sequence as set forth in SEQ ID NO: 205 and the light chain variable region includes a sequence having at least 90% sequence identity to the sequence as set forth in SEQ ID NO: 206.
118. The antibody or fragment thereof of embodiment 47, wherein the heavy chain variable region includes a sequence as set forth in SEQ ID NO: 1311 and the light chain variable region includes the sequence as set forth in SEQ ID NO: 1312; or the heavy chain variable region includes a sequence having at least 90% sequence identity to the sequence as set forth in SEQ ID NO: 1311 and the light chain variable region includes a sequence having at least 90% sequence identity to the sequence as set forth in SEQ ID NO: 1312.
119. The antibody or fragment thereof of embodiment 49, wherein the heavy chain variable region includes a sequence as set forth in SEQ ID NO: 207 and the light chain variable region includes the sequence as set forth in SEQ ID NO: 208; or the heavy chain variable region includes a sequence having at least 90% sequence identity to the sequence as set forth in SEQ ID NO: 207 and the light chain variable region includes a sequence having at least 90% sequence identity to the sequence as set forth in SEQ ID NO: 208.
120. The antibody or fragment thereof of embodiment 51, wherein the heavy chain variable region includes a sequence as set forth in SEQ ID NO: 209 and the light chain variable region includes the sequence as set forth in SEQ ID NO: 210; or the heavy chain variable region includes a sequence having at least 90% sequence identity to the sequence as set forth in SEQ ID NO: 209 and the light chain variable region includes a sequence having at least 90% sequence identity to the sequence as set forth in SEQ ID NO: 210.
121. The antibody or fragment thereof of embodiment 53, wherein the heavy chain variable region includes a sequence as set forth in SEQ ID NO: 211 and the light chain variable region includes the sequence as set forth in SEQ ID NO: 212; or the heavy chain variable region includes a sequence having at least 90% sequence identity to the sequence as set forth in SEQ ID NO: 211 and the light chain variable region includes a sequence having at least 90% sequence identity to the sequence as set forth in SEQ ID NO: 212.
122. The antibody or fragment thereof of embodiment 55, wherein the heavy chain variable region includes a sequence as set forth in SEQ ID NO: 213 and the light chain variable region includes the sequence as set forth in SEQ ID NO: 214; or the heavy chain variable region includes a sequence having at least 90% sequence identity to the sequence as set forth in SEQ ID NO: 213 and the light chain variable region includes a sequence having at least 90% sequence identity to the sequence as set forth in SEQ ID NO: 214.
123. The antibody or fragment thereof of embodiment 57, wherein the heavy chain variable region includes a sequence as set forth in SEQ ID NO: 1313 and the light chain variable region includes the sequence as set forth in SEQ ID NO: 1314; or the heavy chain variable region includes a sequence having at least 90% sequence identity to the sequence as set forth in SEQ ID NO: 1313 and the light chain variable region includes a sequence having at least 90% sequence identity to the sequence as set forth in SEQ ID NO: 1314.
124. The antibody or fragment thereof of embodiment 59, wherein the heavy chain variable region includes a sequence as set forth in SEQ ID NO: 215 and the light chain variable region includes the sequence as set forth in SEQ ID NO: 216; or the heavy chain variable region includes a sequence having at least 90% sequence identity to the sequence as set forth in SEQ ID NO: 215 and the light chain variable region includes a sequence having at least 90% sequence identity to the sequence as set forth in SEQ ID NO: 216.
125. The antibody or fragment thereof of embodiment 61, wherein the heavy chain variable region includes a sequence as set forth in SEQ ID NO: 217 and the light chain variable region includes the sequence as set forth in SEQ ID NO: 218; or the heavy chain variable region includes a sequence having at least 90% sequence identity to the sequence as set forth in SEQ ID NO: 217 and the light chain variable region includes a sequence having at least 90% sequence identity to the sequence as set forth in SEQ ID NO: 218.
126. The antibody or fragment thereof of embodiment 63, wherein the heavy chain variable region includes a sequence as set forth in SEQ ID NO: 219 and the light chain variable region includes the sequence as set forth in SEQ ID NO: 220; or the heavy chain variable region includes a sequence having at least 90% sequence identity to the sequence as set forth in SEQ ID NO: 219 and the light chain variable region includes a sequence having at least 90% sequence identity to the sequence as set forth in SEQ ID NO: 220.
127. The antibody or fragment thereof of embodiment 65, wherein the heavy chain variable region includes a sequence as set forth in SEQ ID NO: 221 and the light chain variable region includes the sequence as set forth in SEQ ID NO: 222; or the heavy chain variable region includes a sequence having at least 90% sequence identity to the sequence as set forth in SEQ ID NO: 221 and the light chain variable region includes a sequence having at least 90% sequence identity to the sequence as set forth in SEQ ID NO: 222.
128. The antibody or fragment thereof of embodiment 67, wherein the heavy chain variable region includes a sequence as set forth in SEQ ID NO: 223 and the light chain variable region includes the sequence as set forth in SEQ ID NO: 224; or the heavy chain variable region includes a sequence having at least 90% sequence identity to the sequence as set forth in SEQ ID NO: 223 and the light chain variable region includes a sequence having at least 90% sequence identity to the sequence as set forth in SEQ ID NO: 224.
129. The antibody or fragment thereof of embodiment 69, wherein the heavy chain variable region includes a sequence as set forth in SEQ ID NO: 225 and the light chain variable region includes the sequence as set forth in SEQ ID NO: 226; or the heavy chain variable region includes a sequence having at least 90% sequence identity to the sequence as set forth in SEQ ID NO: 225 and the light chain variable region includes a sequence having at least 90% sequence identity to the sequence as set forth in SEQ ID NO: 226.
130. The antibody or fragment thereof of embodiment 71, wherein the heavy chain variable region includes a sequence as set forth in SEQ ID NO: 27 and the light chain variable region includes the sequence as set forth in SEQ ID NO: 228; or the heavy chain variable region includes a sequence having at least 90% sequence identity to the sequence as set forth in SEQ ID NO: 227 and the light chain variable region includes a sequence having at least 90% sequence identity to the sequence as set forth in SEQ ID NO: 228.
131. The antibody or fragment thereof of embodiment 73, wherein the heavy chain variable region includes a sequence as set forth in SEQ ID NO: 229 and the light chain variable region includes the sequence as set forth in SEQ ID NO: 230; or the heavy chain variable region includes a sequence having at least 90% sequence identity to the sequence as set forth in SEQ ID NO: 229 and the light chain variable region includes a sequence having at least 90% sequence identity to the sequence as set forth in SEQ ID NO: 230.
132. The antibody or fragment thereof of embodiment 75, wherein the heavy chain variable region includes a sequence as set forth in SEQ ID NO: 231 and the light chain variable region includes the sequence as set forth in SEQ ID NO: 232; or the heavy chain variable region includes a sequence having at least 90% sequence identity to the sequence as set forth in SEQ ID NO: 231 and the light chain variable region includes a sequence having at least 90% sequence identity to the sequence as set forth in SEQ ID NO: 232.
133. The antibody or fragment thereof of embodiment 77, wherein the heavy chain variable region includes a sequence as set forth in SEQ ID NO: 233 and the light chain variable region includes the sequence as set forth in SEQ ID NO: 234; or the heavy chain variable region includes a sequence having at least 90% sequence identity to the sequence as set forth in SEQ ID NO: 233 and the light chain variable region includes a sequence having at least 90% sequence identity to the sequence as set forth in SEQ ID NO: 234.
134. The antibody or fragment thereof of embodiment 79, wherein the heavy chain variable region includes a sequence as set forth in SEQ ID NO: 235 and the light chain variable region includes the sequence as set forth in SEQ ID NO: 236; or the heavy chain variable region includes a sequence having at least 90% sequence identity to the sequence as set forth in SEQ ID NO: 235 and the light chain variable region includes a sequence having at least 90% sequence identity to the sequence as set forth in SEQ ID NO: 236.
135. The antibody or fragment thereof of embodiment 81, wherein the heavy chain variable region includes a sequence as set forth in SEQ ID NO: 237 and the light chain variable region includes the sequence as set forth in SEQ ID NO: 238; or the heavy chain variable region includes a sequence having at least 90% sequence identity to the sequence as set forth in SEQ ID NO: 237 and the light chain variable region includes a sequence having at least 90% sequence identity to the sequence as set forth in SEQ ID NO: 238.
136. The antibody or fragment thereof of embodiment 83, wherein the heavy chain variable region includes a sequence as set forth in SEQ ID NO: 239 and the light chain variable region includes the sequence as set forth in SEQ ID NO: 240; or the heavy chain variable region includes a sequence having at least 90% sequence identity to the sequence as set forth in SEQ ID NO: 239 and the light chain variable region includes a sequence having at least 90% sequence identity to the sequence as set forth in SEQ ID NO: 240.
137. The antibody or fragment thereof of embodiment 85, wherein the heavy chain variable region includes a sequence as set forth in SEQ ID NO: 241 and the light chain variable region includes the sequence as set forth in SEQ ID NO: 242; or the heavy chain variable region includes a sequence having at least 90% sequence identity to the sequence as set forth in SEQ ID NO: 241 and the light chain variable region includes a sequence having at least 90% sequence identity to the sequence as set forth in SEQ ID NO: 242.
138. The antibody or fragment thereof of embodiment 87, wherein the heavy chain variable region includes a sequence as set forth in SEQ ID NO: 243 and the light chain variable region includes the sequence as set forth in SEQ ID NO: 244; or the heavy chain variable region includes a sequence having at least 90% sequence identity to the sequence as set forth in SEQ ID NO: 243 and the light chain variable region includes a sequence having at least 90% sequence identity to the sequence as set forth in SEQ ID NO: 244.
139. The antibody or fragment thereof of embodiment 89, wherein the heavy chain variable region includes a sequence as set forth in SEQ ID NO: 245 and the light chain variable region includes the sequence as set forth in SEQ ID NO: 246; or the heavy chain variable region includes a sequence having at least 90% sequence identity to the sequence as set forth in SEQ ID NO: 245 and the light chain variable region includes a sequence having at least 90% sequence identity to the sequence as set forth in SEQ ID NO: 246.
140. The antibody or fragment thereof of embodiment 91, wherein the heavy chain variable region includes a sequence as set forth in SEQ ID NO: 247 and the light chain variable region includes the sequence as set forth in SEQ ID NO: 248; or the heavy chain variable region includes a sequence having at least 90% sequence identity to the sequence as set forth in SEQ ID NO: 247 and the light chain variable region includes a sequence having at least 90% sequence identity to the sequence as set forth in SEQ ID NO: 248.
141. The antibody or fragment thereof of any of embodiments 1-140, wherein the antibody or fragment thereof further includes the human constant region.
142. The antibody or fragment thereof of embodiment 141, wherein the human constant region includes a human heavy chain constant region and/or a human light chain constant region.
143. The antibody or fragment thereof of embodiment 142, wherein the human heavy chain constant region includes a human IgG1 heavy chain constant region, a human IgG4 heavy chain constant region, a human IgG2 heavy chain constant region, or a human IgG3 heavy chain constant region.
144. The antibody or fragment thereof of embodiment 142, wherein the human light chain constant region includes a human Igκ light chain constant region or a human Igλ light chain constant region.
145. A chimeric antigen receptor (CAR) that, when expressed by a cell, includes an extracellular component linked to an intracellular component by a transmembrane domain, wherein the extracellular component includes the antibody or fragment thereof of any of embodiments 1-144.
146. The CAR of embodiment 145, wherein the intracellular component includes an effector domain including: 4-1BB (CD137), CD3γ, CD3δ, CD3ϵ, CD3ζ, CD27, CD28, DAP10, ICOS, LAG3, NKG2D, NOTCH1, OX40, ROR2, SLAMF1, TCRα, TCRβ, TRIM, Wnt, Zap70, or a combination thereof.
147. The CAR of embodiment 145 or 146, wherein the transmembrane domain includes a transmembrane region of: the α, β or ζ chain of a T-cell receptor; CD28; CD27; CD3; CD45; CD4; CD5; CD8; CD9; CD16; CD22; CD33; CD37; CD64; CD80; CD86; CD134; CD137; CD154; or a combination thereof.
148. The CAR of any of embodiments 145-147, wherein the CAR further includes a spacer region.
149. An engineered T cell receptor (eTCR) including a constant alpha domain (C_{α}), a constant beta domain (C_{β}), and the antibody or fragment thereof of any of embodiments 1-144 linked to the C_{α} domain and/or the C_{β} domain.
150. The eTCR of embodiment 149, wherein the antibody or fragment thereof of embodiment 1 is an scFv.
151. The eTCR of embodiment 149 or 150, wherein the antibody or fragment thereof of embodiment 1 is linked to the C_{α} domain.
152. The eTCR of any of embodiments 149-151, wherein the antibody or fragment thereof of embodiment 1 is linked to the C_{β} domain.
153. The eTCR of embodiment any of embodiments 149-152, wherein one antibody or fragment thereof of any of embodiments 1-144 is linked to the C_{α} domain and one antibody or fragment thereof of any of embodiments 1-144 is linked to the C_{β} domain.
154. A cell genetically modified to express the antibody or fragment thereof of any of embodiments 1-144, the CAR of embodiment 145-148, or the eTCR of embodiment 149-153.
155. The cell of embodiment 154, wherein the cell is an immune cell.
156. The cell of embodiment 155, wherein the immune cell is a B cell, T cell, natural killer cell, or macrophage.
157. The cell of any of embodiments 154-156, wherein the cell is a B cell genetically modified to express the antibody or fragment thereof of any of embodiments 1-144.
158. A single-chain variable fragment (scFv) including the light chain variable region and the heavy chain variable region of an antibody or fragment thereof of any of embodiments 1-144, linked by a flexible linker.
159. A multi-specific binding domain molecule including at least two binding domains wherein at least one binding domain includes the antibody or fragment thereof of any of embodiments 1-144.
160. The multi-specific binding domain molecule of embodiment 159, wherein the multi-specific binding domain molecule includes an immune cell engaging molecule.
161. The multi-specific binding domain molecule of embodiment 160, wherein the immune cell engaging molecule activates a B cell, T cell, natural killer (NK) cell, or macrophage.
162. The multi-specific binding domain molecule of embodiment 161, wherein the T cell is a CD3 T cell, a CD4 T cell, a CD8 T cell, a central memory T cell, an effector memory T cell, and/or a naïve T cell.
163. The multi-specific binding domain molecule of any of embodiments 160-162, wherein a binding domain of the immune cell engaging molecule binds CD40, ICOS, IL21R, IL4R, CD3, CD28, CD8, NKG2D, CD8, CD16, KIR2DL4, KIR2DS1, KIR2DS2, KIR3DS1, NKG2C, NKG2E, NKG2D, NKp30, NKp44, NKp46, NKp80, DNAM-1, CD11b, CD11c, CD64, CD68, CD119, CD163, CD206, CD209, F4/80, IFGR2, Toll-like receptors 1-9, IL-4Rα, or MARCO.
164. The multi-specific binding domain molecule of any of embodiments 159-163, wherein the at least two binding domains include at least two copies of the antibody or fragment thereof of any of embodiments 1-144.
165. The multi-specific binding domain molecule of any of embodiments 159-164, wherein the at least two copies are joined by a protein linker.
166. The multi-specific binding domain molecule of embodiment 165, wherein the protein linker is a Gly-Ser linker.
167. The multi-specific binding domain molecule of embodiment 166, wherein the Gly-Ser linker is (GlyₓSer_{y})ₙ wherein x and y are independently an integer from 0 to 10 provided that x and y are not both 0 and wherein n is an integer of 1, 2, 3, 4, 5, 6, 7, 8, 9 or 10.
168. The multi-specific binding domain molecule of any of embodiments 159-167, including 2, 3, 4, 5, 6, 7, 8, 9, or 10 copies of the antibody or fragment thereof of any of embodiments 1-144.
169. The multi-specific binding domain molecule of any of embodiments 159-168, wherein the multi-specific binding domain molecule is a dimer, trimer, tetramer, pentamer, hexamer, or heptamer.
170. A conjugate including the antibody or fragment of any of embodiments 1-144 linked to an immunotoxin, a drug, a detectable label, or a radioisotope.
171. The conjugate of embodiment 170, wherein the immunotoxin includes a plant toxin or bacterial toxin.
172. The conjugate of embodiment 171, wherein the plant toxin includes ricin, abrin, mistletoe lectin, modeccin, pokeweed antiviral protein, saporin, Bryodin 1, bouganin, or gelonin.
173. The conjugate of embodiment 171 or 172, wherein the bacterial toxin includes diphtheria toxin or Pseudomonas exotoxin.
174. The conjugate of any of embodiments 170-173, wherein the drug includes a cytotoxic drug.
175. The conjugate of embodiment 174, wherein the cytotoxic drug includes actinomycin D, anthracycline, auristatin, calicheamicin, camptothecin, CC1065, colchicin, cytochalasin B, daunorubicin, 1-dehydrotestosterone, dihydroxy anthracinedione, dolastatin, doxorubicin, duocarmycin, elinafide, emetine, ethidium bromide, etoposide, gramicidin D, glucocorticoids, lidocaine, maytansinoid, mithramycin, mitomycin, mitoxantrone, nemorubicin, PNU-159682, procaine, propranolol, puromycin, pyrrolobenzodiazepine, taxane, taxol, tenoposide, tetracaine, trichothecene, vinblastine, vinca alkaloid, or vincristine.
176. The conjugate of any of embodiments 170-175, wherein the detectable label includes a fluorescent label, a chemiluminescent label, a spectral colorimetric label, an enzymatic label, or an affinity tag.
177. The conjugate of embodiment 176, wherein the fluorescent label includes blue fluorescent protein, cyan fluorescent protein, green fluorescent protein, luciferase, orange fluorescent protein, red fluorescent protein, far red fluorescent protein, or yellow fluorescent protein.
178. The conjugate of embodiment 176 or 177, wherein the chemiluminescent label includes lucigenin, luminol, luciferin, isoluminol, theromatic acridinium ester, imidazole, acridinium salt, or oxalate ester.
179. The conjugate of any of embodiments 176-178, wherein the spectral colorimetric label includes colloidal gold.
180. The conjugate of any of embodiments 176-179, wherein the enzymatic label includes malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase, or acetylcholinesterase.
181. The conjugate of any of embodiments 176-180, wherein the affinity tag includes a tag with a sequence as set forth in SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, SEQ ID NO: 187, SEQ ID NO: 188, or SEQ ID NO: 189.
182. The conjugate of any of embodiments 170-181, wherein the radioisotope includes ²²⁸Ac, ¹¹¹Ag, ¹²⁴Am, ⁷⁴As, ²¹¹At, ²⁰⁹At, ¹⁹⁴Au, ¹²⁸³Ba, ⁷Be, ²⁰⁶Bi, ²⁴⁵Bk, ²⁴⁶Bk, ⁷⁶Br, ¹¹C, ¹⁴C, ⁴⁷Ca, ²⁵⁴Cf, ²⁴²Cm, ⁵¹Cr, ⁶⁷Cu, ¹⁵³Dy, ¹⁵⁷Dy, ¹⁵⁹Dy, ¹⁶⁵Dy, ¹⁶⁶Dy, ¹⁷¹Er, ²⁵⁰Es, ²⁵⁴Es, ¹⁴⁷Eu, ¹⁵⁷Eu, ⁵²Fe, ⁵⁹Fe, ²⁵¹Fm, ²⁵²Fm, ²⁵³Fm, ⁶⁶Ga, ⁷²Ga, ¹⁴⁶Gd, ¹⁵³Gd, ⁶⁸Ge, ³H, ¹⁷⁰Hf, ¹⁷¹Hf, ¹⁹³Hg, ¹⁹³mHg, ¹⁶⁰mHo, ¹³⁰I, ¹³¹I, ¹³⁵I, ¹¹⁴mIn, ¹⁸⁵Ir, ⁴²K, ⁴³K, ⁷⁶Kr, ⁷⁹Kr, ⁸¹mKr ¹³²La, ²⁶²Lr, ¹⁶⁹Lu, ¹⁷⁴mLu, ¹⁷⁶mLu, ²⁵⁷Md, ²⁶⁰Md, ²⁸Mg, ⁵²Mn, ⁹⁰Mo, ²⁴Na, ⁹⁵Nb, ¹³⁸Nd, ⁵⁷Ni, ⁶⁶Ni, ²³⁴Np, ¹⁵O, ¹⁸²Os, ¹⁸⁹mOs, ¹⁹¹Os, ³²P, ²⁰¹Pb, ¹⁰¹Pd, ¹⁴³Pr, ¹⁹¹Pt, ²⁴³Pu, ²²⁵Ra, ⁸¹Rb, ¹⁸⁸Re, ¹⁰⁵Rh, ²¹¹Rn, ¹⁰³Ru, ³⁵S, ⁴⁴Sc, ⁷²Se, ¹⁵³Sm, ¹²⁵Sn, ⁹¹Sr, ¹⁷³Ta, ¹⁵⁴Tb, ¹²⁷Te, ²³⁴Th, ⁴⁵Ti, ¹⁶⁶Tm, ²³⁰U, ²³⁷U, ²⁴⁰U, ⁴⁸V, ¹⁷⁸W, ¹⁸¹W, ¹⁸⁵W, ¹²⁵Xe, ¹²⁷Xe, ¹³³Xe, ¹³³mXe, ¹³⁵Xe, ⁸⁵mY, ⁸⁶Y, ⁹⁰Y, ⁹³Y, ¹⁶⁹Yb, ¹⁷⁵Yb, ⁶⁵Zn, ⁷¹mZn, ⁸⁶Zr, ⁹⁵Zr, or ⁹⁷Zr.
183. A detection assay including an antibody or fragment thereof of any of embodiments 1-144, an scFv of embodiment 158, or a conjugate of any of embodiments 170-182 linked to a solid support.
184. The detection assay of embodiment 183, wherein the solid support includes slides, wells, chips, pins, filters, beads, or membrane.
185. The detection assay of embodiment 183, wherein the solid substrate includes polystyrene, glass, gold, nylon, nitrocellulose, polyvinylidene fluoride (PVDF), paper, or latex.
186. The detection assay of any of embodiments 183-185, wherein the antibody or fragment thereof, scFv, or conjugate is linked to the solid support via a covalent interaction, a noncovalent interaction, or passive binding.
187. A nucleic acid encoding an antibody or fragment thereof of any of embodiments 1-144, the scFv of embodiment 158, the multi-specific binding domain molecule of any of embodiments 159-169, or the conjugate of any of embodiments 170-182.
188. A composition including the antibody or fragment thereof of any of embodiments 1-144, the scFv of embodiment 158, the multi-specific binding domain molecule of any of embodiments 159-169, the conjugate of any of embodiments 170-182, or a nucleic acid of embodiment 187, and a pharmaceutically acceptable carrier.
189. A nucleic acid encoding the antibody or fragment thereof of any of embodiments 1-144, CAR of any of embodiments 145-148, the eTCR of any of embodiments 149-153, the scFv of embodiment 158, or the multi-specific binding domain molecule of any of embodiments 159-169.
190. A formulation including the cell of any of embodiments 154-157 or the nucleic acid of embodiment 189, and a pharmaceutically acceptable carrier.
191. A kit including the antibody or fragment thereof of any of embodiments 1-144, CAR of any of embodiments 145-148, the eTCR of any of embodiments 149-153, the scFv of embodiment 158, the multi-specific binding domain molecule of any of embodiments 159-169, the conjugate of any of embodiments 170-182, or the nucleic acid of embodiment 187 or 189.
192. A method of treating a subject in need thereof including administering a therapeutically effective amount of the composition of embodiment 188 and/or the formulation of embodiment 190 thereby treating the subject in need thereof.
193. The method of embodiment 192, wherein the therapeutically effective amount provides a prophylactic or a therapeutic treatment against a Merkel cell carcinoma.
194. The method of embodiment 192 or 193, wherein the administering is through intravenous, intradermal, intraarterial, intranodal, intravesicular, intrathecal, intraperitoneal, intraparenteral, intranasal, intralesional, intramuscular, oral, intrapulmonary, subcutaneous, or sublingual administering.
195. A method of detecting a T antigen of the Merkel cell polyomavirus (T antigen) including administering to a subject or a biological sample derived from the subject a detection molecule including the composition of embodiment 188 and detecting the detection molecule.
196. The method of embodiment 195, wherein the detecting includes imaging.
197. The method of embodiment 195 or 196, further including diagnosing the subject based on the detecting.
198. The method of embodiment 197, wherein the diagnosing includes determining the level of expression of the T antigen based on a signal from the detectable molecule and comparing the level of expression of the detectable molecule to a reference level.
199. The method of embodiment 198, wherein the reference level is the level of the signal from a tissue without a T antigen-expressing cell.
200. The method of embodiment 198, wherein the reference level is the level of the signal from the subject or the biological sample derived from the subject at a different time point.
201. The method of any of embodiments 195-200, wherein the biological sample derived from the subject includes a tissue biopsy, a tumor tissue biopsy, or a skin biopsy.
202. A method, including:
   obtaining a sample derived from a tumor-infiltrated lymph node derived from a subject;
   assessing the sample for the presence of T-Ag specific germinal center B cells; and
   administering an MCC treatment to the subject if T-Ag specific germinal center B cells are not present in the sample.

(xix) Experimental Example. Abstract. 80% of Merkel cell carcinoma (MCC) cases in the US arise from expression of Merkel cell polyomavirus (MCPyV)-derived oncoproteins called T-antigens. Tumorigenesis occurs after a truncated form of MCPyV T-antigen DNA is inserted into the cell's genome and expression drives MCC. Despite these oncoproteins being expressed intracellularly, most T-antigen-driven MCC patients produce T-antigen-specific serum antibodies near the time of diagnosis, which are rarely found in UV-driven MCC cases or individuals without MCC despite the high prevalence of MCPyV infection in the general population. While rising T-antigen-specific serum antibody levels are used clinically to detect MCC recurrence after treatment, it is unknown how disease progression or control is impacted by T-antigen-specific antibodies or the B cells that produce them. Given that MCC progression occurs within 18 months in 37% of patients following the standard of care therapy of tumor excision plus local radiation, improved therapies would benefit thousands of people per year. While PD-1 blockade immunotherapies have had remarkable success treating advanced and later stage MCC, additional options are needed for the 50% of patients who do not durably respond to these therapies. Additional biomarkers are also needed to better identify high risk patients so that more aggressive approaches can be used.

In this experimental example, analysis of the humoral immune response shows that the presence of T-antigen-specific germinal center B cells in tumor-infiltrated lymph node samples predicts a lack of MCC progression. Based on these results, B cells expressing somatically hypermutated antibodies with high affinity for T-antigen enhance MCC tumor-killing by T cells to limit disease progression.

Introduction. MCC is a rare and deadly skin cancer resulting in an estimated 33-46% mortality amongst the 3,000 MCC cases diagnosed in the US each year (Nghiem et al., Future oncology 2017; 13(14):1263-1279; Chan et al., J Immunother Cancer 2018; 6(1):23). One reason for this high mortality is that MCC is an aggressive cancer that has often infiltrated the lymph node or spread to a distant site at the time of diagnosis (Harms et al., Ann Surg Oncol 2016; 23(11):3564-71). A second reason for high mortality is that after initial treatment, 40% of patients experience progressive MCC within five years, with tumors often detected at sites distinct from initial presentation (Nghiem et al., Future oncology 2017; 13(14):1263-1279; Chan et al., J Immunother Cancer 2018; 6(1):23; McEvoy et al., JAMA Dermatol 2022; 158(4):382-89). A final reason is that MCC typically occurs in people over the age of 65 and often associates with immunocompromising conditions (Harms et al., Ann Surg Oncol 2016; 23(11):3564-71; McEvoy et al., JAMA Dermatol 2022; 158(4):382-89), suggesting a role for the immune system in control of MCC.

Modulating immune responses has already made an impact for MCC patients. The first impact is success of PD-1 immune checkpoint blockade therapy to treat metastatic and advanced MCC (Truong et al., Australas J Dermatol 2022; 63(1):e6-e12). Overall, around half of MCC patients have a long-term benefit from PD-1 blockade immunotherapy, one of the highest response rates of all solid tumors (Nghiem et al., J Immunother Cancer 2021; 9(4)). Nevertheless, predictive biomarkers and additional treatment options are needed for the half of advanced MCC cases that progress despite PD-1 immunotherapy. This experimental example explores the factors mediating MCC progression despite PD-1 blockade immunotherapy, which could be used as predictive biomarkers and/or lead to the development of curative therapies.

Studying immune responses resulted in the discovery that rising levels of MCPyV T-antigen (T-Ag)-specific antibodies in the blood of patients revealed MCC recurrence prior to the detection of tumor (Paulson et al., Cancer research 2010; 70(21):8388-97; Paulson et al., Cancer 2017; 123(8):1464-74). This discovery allows for earlier utilization of therapies like PD-1 blockade to combat MCC recurrences in patients producing these antibodies. This experimental example explores the role of T-Ag-specific antibodies and B cells in MCC both as predictive biomarkers, and as potential targets for next generation immunotherapy.

This experimental example describes the analysis of B and T cell responses specific for the same oncoprotein across the entire patient cohort. Studying oncoprotein-specific B and T cell responses is often challenging for tumors where oncoproteins and tumor antigens vary from case-to-case due to patient-specific mutations, or immune responses are poor due to immune tolerance resulting from the oncoprotein being a self-antigen. Around 80% of MCC cases in the US are driven by the expression of MCPyV T-Ags (Feng et al., Science 2008; 319(5866):1096-100; Paik et al., Human pathology 2011; 42(10):1385-90). Most people are exposed to MCPyV during childhood and the virus often remains detectable on the skin throughout their lives (Martel-Jantin C, et al., Journal of clinical virology 2013; 58(1):288-91; Signorini et al., Journal of clinical virology 2014; 61(4):565-70; Harms et al., Nat Rev Clin Oncol 2018; 15(12):763-76). MCPyV T-Ag is required for viral replication but appears harmless during natural infection. In rare occasions, a truncated portion of T-Ag DNA is inserted into the genome and the expression results in T-Ag oncoproteins driving proliferation of MCC (FIG. 1) (Kwun et al., J Virol 2009; 83(23):12118-28; Shuda et al., J Clin Invest 2011; 121(9):3623-34; Houben et al., International journal of cancer 2012; 130(4):847-56; Chang & Moore, Annual review of pathology 2012; 7:123-44; Wu et al., Intervirology 2015; 58(6):382-5; Hesbacher et al., Oncotarget 2016; 7(22):32956-68; Schrama et al., International journal of cancer 2016; 138(5):1153-62; Wu et al., Virus genes 2016; 52(3):397-9; Goh et al., Oncotarget 2016; 7(3):3403-15). This form of MCC is called virus positive-MCC (VP-MCC), to indicate that these cancers are driven by expression of the virus-derived truncated T-Ag. Additionally, T-Ag-driven MCC tumors have very low mutation rates (Goh et al., Oncotarget 2016; 7(3):3403-15; Harms et al., Cancer research 2015; 75(18):3720-27; Wong et al., Cancer research 2015; 75(24):5228-34) and appear reliant on expression of T-Ag to maintain proliferation (Houben et al., International journal of cancer 2012; 130(4):847-56; Houben et al., J Virol 2010; 84(14):7064-72; Shuda et al., The Journal of investigative dermatology 2014; 134(5):1479-81). Based on these results, VP-MCC tumors are unlikely to escape T-Ag-directed therapies through T-Ag inactivation, which is often found for therapies targeting other tumor antigens.

Recent evidence from other cancer types has suggested a strong link between the presence of tertiary lymphoid structures containing B cells in tumors and responsiveness to PD-1 blockade therapies (Griss et al., Nat Commun 2019; 10(1):4186; Helmink et al., Nature 2020; 577(7791):549-55; Cabrita et al., Nature 2020; 577(7791):561-65; Petitprez et al., Nature 2020; 577(7791):556-60; Ng et al., Nature 2023; 616:563-573). A link between tertiary lymphoid structures and VP-MCC progression has not been detected (Nakamura M, et al., Front Oncol 2022; 12:811586), however PD-1 blockade therapy was not assessed. This experimental example explores whether T-Ag-specific B cells are predictive of PD-1 blockade responsiveness in VP-MCC. In this experimental example, tumor samples are interrogated for evidence of antigen-specific B and T cell interactions, which is difficult to address in tumors with variable tumor antigens.

This experimental example utilizes the newly developed “SLAP” model of MCC described in Verhaegen et al., J Clin Invest 2022; 132(7) to probe the mechanisms used by immune cells to control MCC. SLAP mice generate MCC tumors in response to tamoxifen and doxycycline by inducing the expression of the small and truncated large T-Ag, the Merkel cell-driving transcription factor Atoh1, and the inactivation of Trp53 in K5-expressing epidermal cells (Verhaegen et al., supra). SLAP mice develop skin tumors 11-22 weeks after induction that phenotypically and transcriptionally resemble human MCC (Verhaegen et al., supra). With this model, the role of T-Ag-specific germinal center B cell responses is explored in this experimental example.

Results. Detection of T-Ag-specific serum antibodies. Most patients with VP-MCC have detectable T-Ag-specific antibodies in blood around the time of diagnosis (Paulson et al., Cancer research 2010; 70(21):8388-97; Paulson et al., Cancer 2017; 123(8):1464-74). T-Ag-specific antibody titers vary widely from patient-to-patient and usually decline rapidly after treatment with surgery+/−radiation (Paulson et al., supra; Paulson et al., supra). After this initial decline in T-Ag-specific titers, patients exhibiting rising titers were experiencing an MCC recurrence that was not yet detectable by other methods (Paulson et al., supra; Paulson et al., supra). Rises in T-Ag serum titers are now used clinically per National Comprehensive Center Network guidelines to detect VP-MCC recurrences (Paulson et al., Cancer 2017; 123(8):1464-74), allowing for earlier initiation of treatments such as PD-1 blockade immunotherapy. There are no other known examples of tumor-specific antibody levels playing such an essential role in care of patients (Lang Kuhs et al., Cancer 2017; 123(22):4382-90).

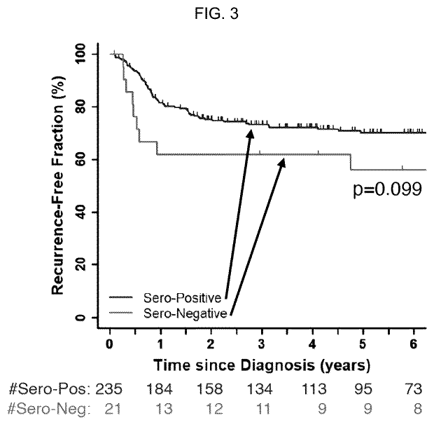

The level of T-Ag-specific serum antibodies near the time of diagnosis is not used as a predictive biomarker of MCC progression. However, results from the Bioinformatics & Biostatistics Core from the 256 VP-MCC patient cohort suggests that individuals with undetectable serum T-Ag-specific antibodies trended towards higher risk of MCC progression compared to their counterparts with detectable levels (FIG. 3). Undetectable levels of T-Ag-specific serum antibodies did not appear to be the result of a defective immune system, since individuals with known immunocompromising conditions such as HIV/AIDS or hematologic malignancies were excluded from this data set. These data may suggest a link between T-Ag-specific antibody responses and non-progressive MCC.

Antigen tetramers sensitively identify T-Ag-specific B cells. Most T-Ag-specific serum antibodies target a small 78 amino acid “common” region that is shared between the large and small T-Ag oncoproteins expressed in MCC (Paulson et al., Cancer research 2010; 70(21):8388-97). Given the dominance of the antibody response towards this common region, this experimental example focuses on this portion of the T-Ag to focus on the most relevant B cells.

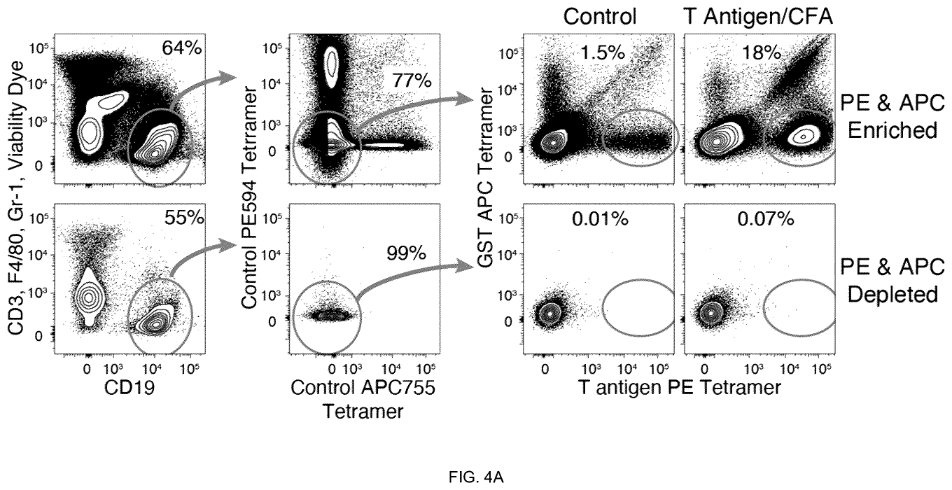

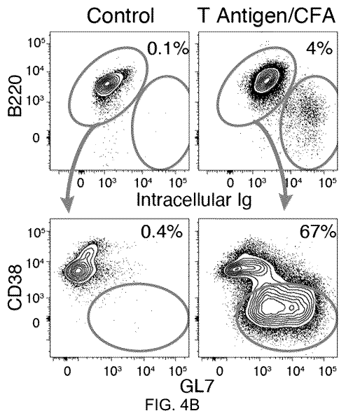

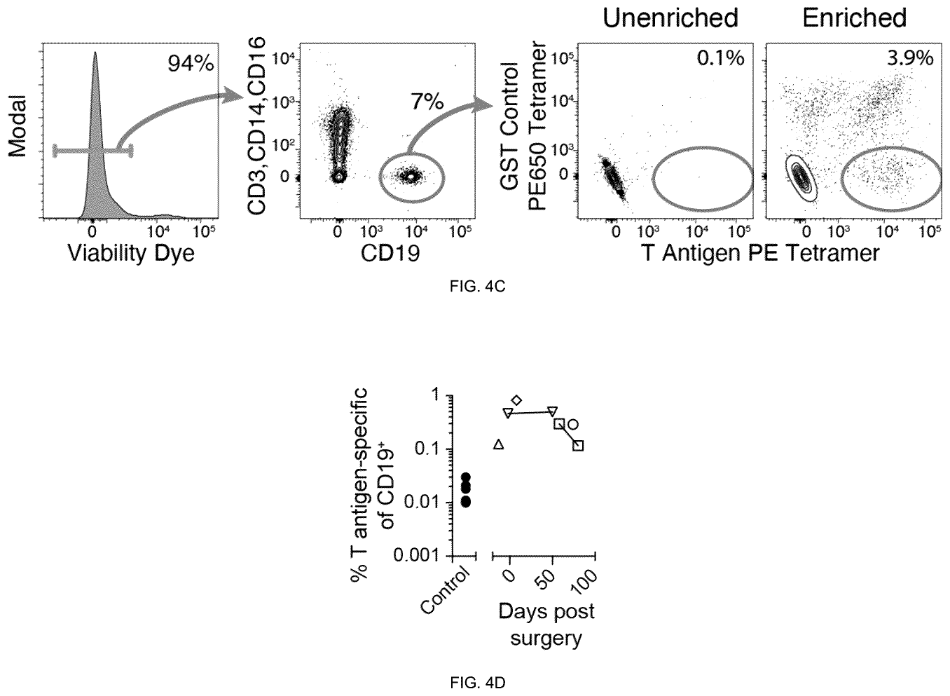

To further understand antibody response to MCC, a fluorescent common T-Ag tetramer was created to identify T-Ag-specific B cells using flow cytometry based upon their expertise producing and validating tetramers (Taylor et al., The Journal of experimental medicine 2012; 209(11):2065-77; Spanier et al., Nat Commun 2016; 7:11804; Bancroft et al., The Journal of experimental medicine 2019; 216(10):2331-2347; Steach et al., J Immunol 2020; 204(3):498-509; Boonyaratanakornkit et al., MAbs 2021; 13(1):1912884; Caban et al., Nat Commun 2023; 14(1):798; Fitzpatrick et al., J Immunol 2023; 210(8):1156-1165). The T-Ag-tetramer was initially validated by confirming that the population of T-Ag tetramer-binding B cells expanded robustly and included both germinal center B cells and antibody secreting cells in animals injected with T-Ag in complete Freund's adjuvant (CFA) (FIGS. 4A, 4B). In these experiments, tetramer-binding B cells were enriched prior to analysis using anti-PE and anti-APC microbeads which allow for detection of tetramer-binding cells in unimmunized controls (Taylor et al., The Journal of experimental medicine 2012; 209(11):2065-77; Bancroft et al., The Journal of experimental medicine 2019; 216(10):2331-2347; Steach et al., J Immunol 2020; 204(3):498-509; Fitzpatrick et al., J Immunol 2023; 210(8):1156-1165) (FIG. 4A). Control APC-DyLight 755 (APC755), PE-DyLight 594 (PE594), and GST tag tetramers were also included to exclude B cells specific for the streptavidin-fluorochrome (Taylor et al., The Journal of experimental medicine 2012; 209(11):2065-77) and the GST tag added to aid T-Ag purification.

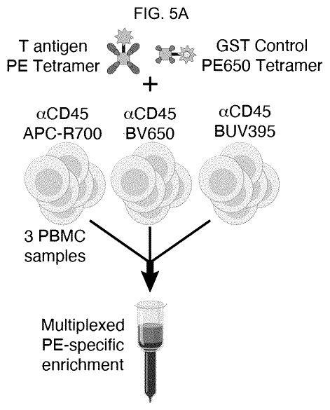

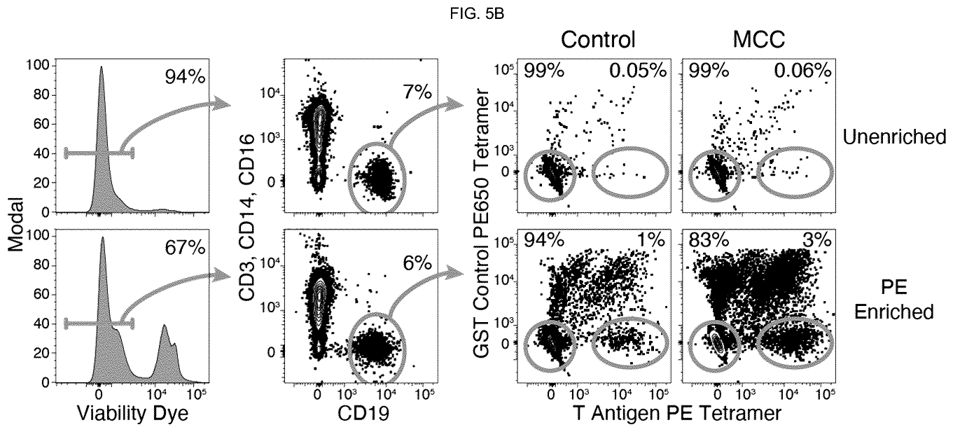

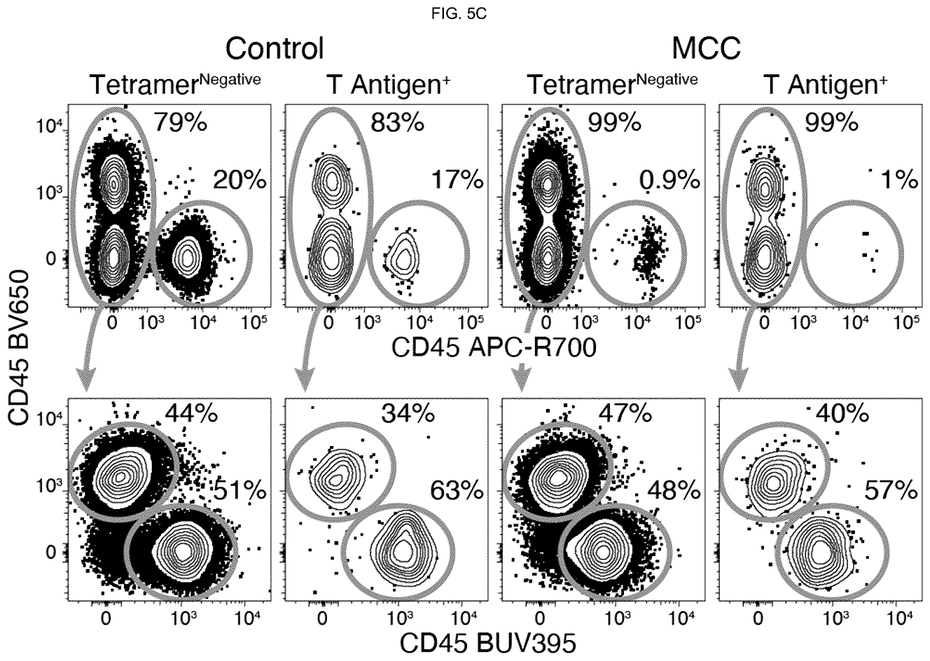

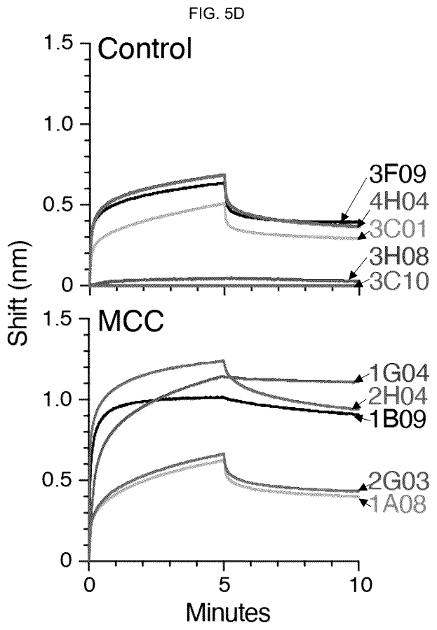

T-Ag tetramers were also bound by human B cells from VP-MCC PBMC samples (FIG. 4C). In these experiments individual T-Ag tetramer-binding human B cells were sorted and paired and heavy and light chain antibody sequences determined using RT-PCR followed by sequencing, as described in Bancroft et al., The Journal of experimental medicine 2019; 216(10):2331-2347; Steach et al., J Immunol 2020; 204(3):498-509; Boonyaratanakornkit et al., MAbs 2021; 13(1):1912884; Caban et al., Nat Commun 2023; 14(1):798; and Fitzpatrick et al., J Immunol 2023; 210(8):1156-1165. Five paired heavy and light chain sequences were expressed as secreted antibody and all five derived from VP-MCC PBMCs bound to T-Ag when assessed using BioLayer Interferometry (FIG. 5D). The antibodies 1G04, 2H04 and 1B09, displaying higher levels of binding to T-Ag by BioLayer Interferometry, contained 27-29 heavy chain nucleotide mutations, suggesting affinity maturation in a germinal center. In contrast, 2G03 and 1A08 had no mutated antibody heavy chain nucleotides and bound T-Ag more moderately by BioLayer Interferometry (FIG. 5D). Analysis of the frequency of T-Ag-specific B cells in PBMC samples from 5 VP-MCC patients at various time points before or after surgery revealed a 10-fold expansion compared to PBMCs from healthy controls (FIG. 4D). Together, these results indicate the ability to detect rare T-Ag-specific B cells with high specificity and suggested the presence of a T-Ag-specific germinal center response during MCC.

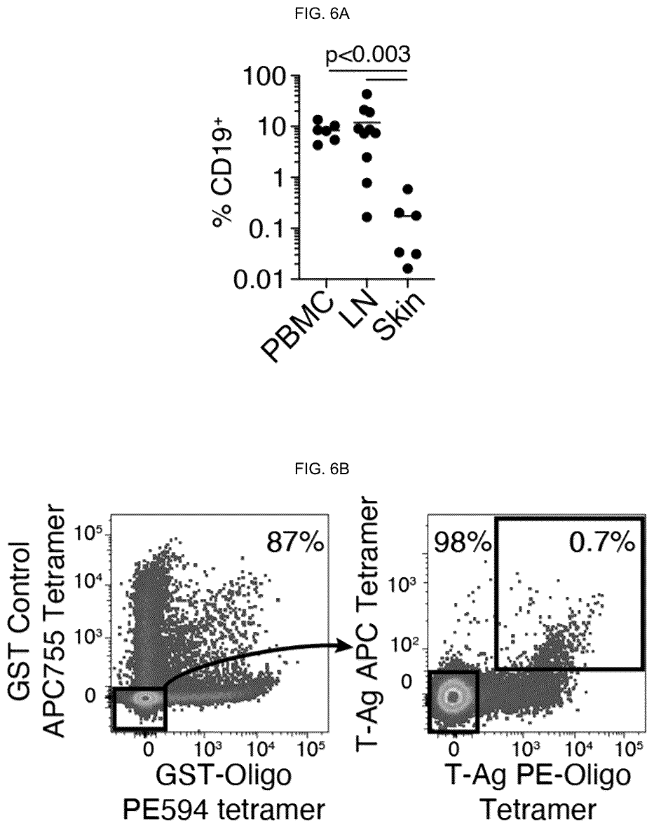

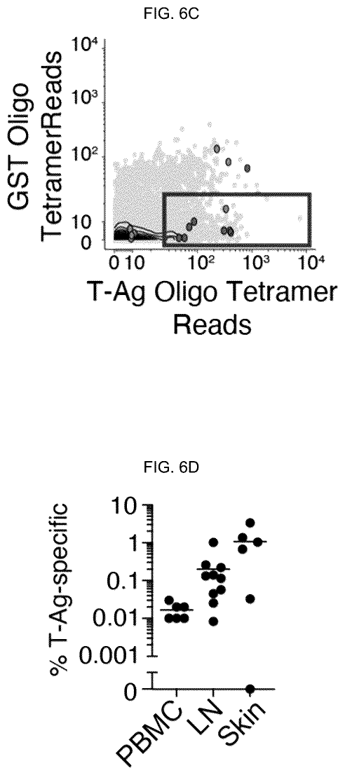

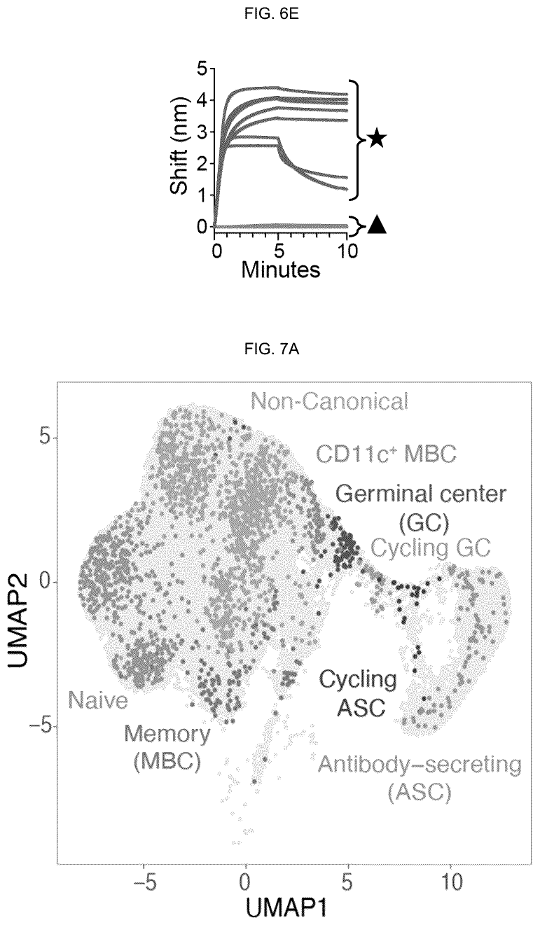

Assessment of T-Ag-specific B cells using 10× single cell sequencing. To accommodate higher dimensional analyses of RNA expression, cell surface phenotype, and paired antibody heavy and light chain sequences for single cells, co-labeling was performed with two fluorescently labeled T-Ag tetramers, one which also included an oligo barcode compatible with RNA sequencing using 10× technologies. Using this approach, B cells accounted for 0.2-43% of cells in tumor-infiltrated LNs and 0.02-0.6% in skin tumors (FIG. 6A). Importantly, the frequency of B cells binding T-Ag tetramer in these samples was only 0.008-3%, with a trend towards a higher frequency in skin tumors (FIGS. 5B-5D), suggesting that most B cells in these samples are not specific for T-Ag. It should be noted that some B cells in these samples could be specific for regions of the large and small T-Ags not included in the tetramer. However, these populations are expected to be very small given that less than 10% of T-Ag-specific serum antibody reacts with the regions unique to the large and small T-Ags (Paulson et al., Cancer research 2010; 70(21):8388-97). Like FIGS. 4A-4D, antibodies were cloned from T-Ag tetramer-binding B cells from tumor samples and 8/9 bound well to T-Ag when assessed via BioLayer Interferometry (FIG. 6E). In this experimental example, the conservative gate was used in FIG. 6B. Together, these data highlight the ability to assess T-Ag-specific B cells in MCC tumor samples.

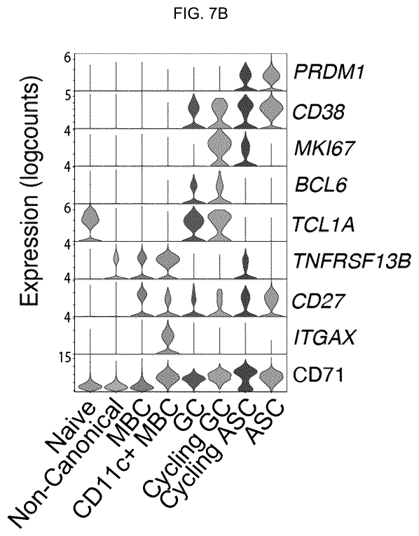

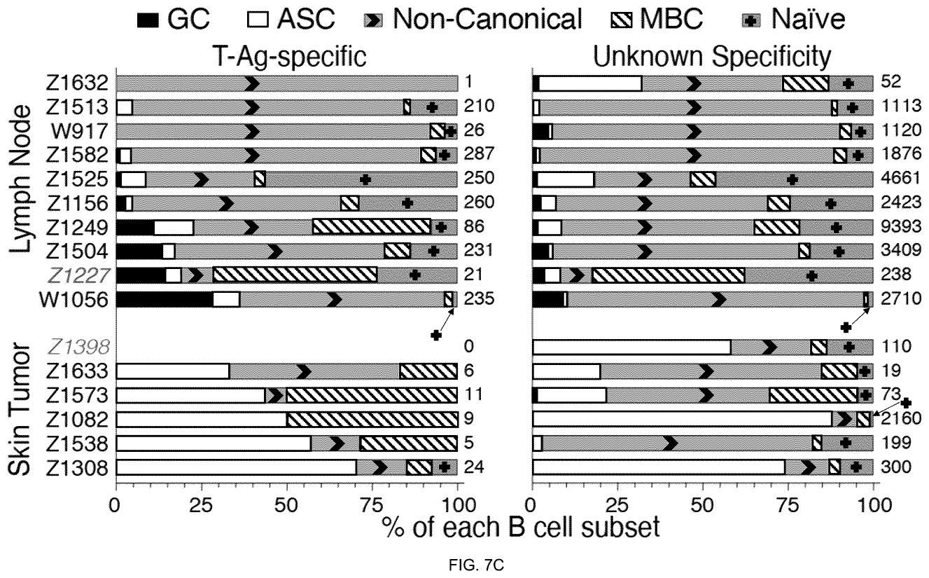

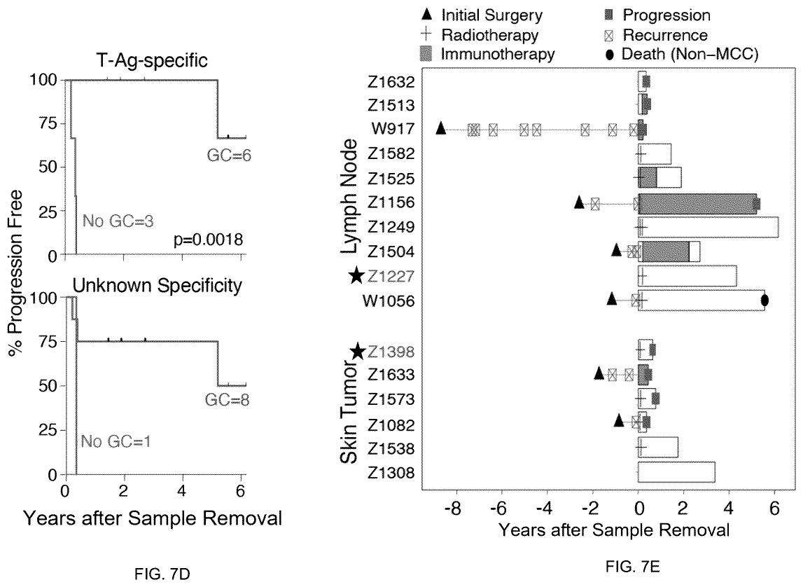

The presence of T-Ag-specific germinal center B cells in tumor samples predicts non-progressive MCC. Having validated the RNA sequencing-based approach, the phenotype of T-Ag-specific B cells and their intratumoral counterparts with unknown specificity were next analyzed. Using UMAP clustering, clusters corresponding to naïve, memory, germinal center, and antibody-secreting cells were detected, along with cells that clustered outside of these canonical subsets that need further characterization since they lacked expression of many hallmark genes associated with the labeled subsets (FIGS. 7A, 7B). This non-canonical population also lacked expression of CD71, which has previously associated with “activated” B cells (Ellebedy et al., Nature immunology 2016; 17(10):1226-34; Wieland et al., Nature 2021; 597(7875):274-78).

Having detected somatic hypermutation in T-Ag-specific antibodies assessed in FIGS. 4A-4D, the analyses in this experimental example have focused on T-Ag-specific germinal center B cells, which were detected in 6/9 of the tumor-infiltrated lymph node samples analyzed and comprised 1-30% of the T-Ag-specific population when detected (FIG. 7C). T-Ag-specific germinal center B cells were not detected in any of the six skin tumors analyzed, though these analyses are limited due to the low numbers of T-Ag-specific B cells in these samples (FIG. 7C). Amongst B cells of unknown specificities, germinal center B cells were detected in 8/9 tumor infiltrated lymph node samples and comprised 0.2-10% of the cells when detected (FIG. 7C). Remarkably, all six patients in which T-Ag-specific germinal center B cells were detected did not experience MCC progression for over 4 years after the analyzed tumors were removed, with one patient experiencing a recurrence 5 years after tumor removal and treatment (FIG. 7D). Strikingly, all three patients in which T-Ag-specific germinal center B cells were not detected in tumor-infiltrated lymph nodes exhibited MCC progression within a year of analysis (FIG. 7D). In contrast, analyzing B cells of unknown specificities did not reveal an association between germinal center B cells and MCC progression (FIG. 7D). This latter result highlights the importance of assessing B cells specific for a known tumor oncoprotein, which only represented 0.008-3% of B cells in these samples. These data indicate that oncoprotein-specific B cell:T cell interactions help protect against MCC progression.

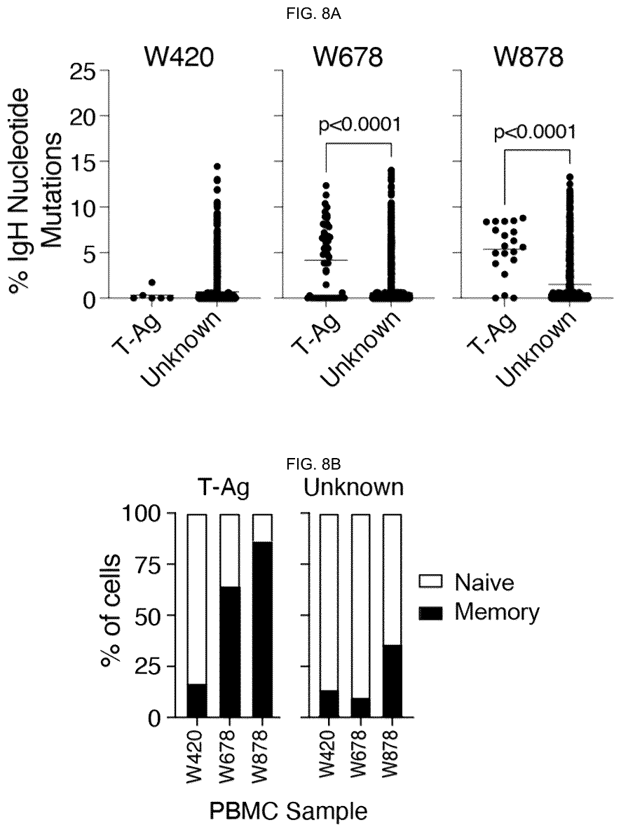

In this experimental example, higher frequencies of antibody heavy chain nucleotide mutations were found in T-Ag-specific B cells in two out of three patient PBMC samples compared to their counterparts of unknown specificity (FIG. 8A). Based on these results, this increase was the result of the high frequency of memory B cells in the T-Ag-specific population from these samples (FIG. 8B). This data indicates the feasibility of assessing somatic hypermutation in T-Ag-specific B cells from MCC PBMC samples.

As described, 50% of patients do not exhibit a durable response to PD-1 blockade immunotherapies and instead exhibit progressive MCC. Analysis of these tumors revealed that T-Ag-specific germinal center B cells could be detected in two out of four samples analyzed. Like analysis of primary tumor samples, two patients with detectable T-Ag-specific germinal center B cells responded to PD-1 blockade therapies and did not exhibit MCC progression, while the other progressed 5 years later. In contrast, the three patients with undetectable T-Ag-specific germinal center B cells exhibited progressive MCC within 1.5 years (FIG. 7E).

MCPyV T-Ag-specific T cell responses association with MCC outcome. Strong B cell responses often require ongoing interactions with CD4+ follicular helper T cells (T_{FH}). T_{FH} cells are often associated with germinal center B cells, but only a subset of T_{FH} cells are found within germinal centers, with other T_{FH} cells interacting with other subtypes of B cells outside of B cells. While most cancer immunology studies have focused on CD8+ T cells, CD4+ helper T cells have also shown therapeutic efficacy in several cancer types (Tran et al., Science 2014; 344(6184):641-5; Hunder et al., The New England journal of medicine 2008; 358(25):2698-703; Veatch et al., J Clin Invest 2018; 128(4):1563-68; Veatch et al., Cancer Cell 2022; 40(4):393-409 e9). While MCC cells are typically HLA class II negative, MCC are infiltrated by monocyte-lineage cells and B cells that are high in class II and could serve as antigen-presenting cells. Indeed, IFN can induce class II expression on MCC cells in vitro (Efficient generation of MCC oncoprotein-specific CD4+ T cells for potential adoptive immunotherapy. American Association of Immunology; 2018). Further, intralesional IL-12 and TLR and STING agonists as IFN-inducers boosted CD4+ infiltration and clinical responses (Bhatia et al., American Society for Clinical Oncology. Chicago, IL, 2016; Bhatia et al., Clin Cancer Res 2018; Submitted; Bhatia et al., European Journal of Cancer 2015; 51 Supplement 3:S104).

The mechanisms by which B cell and T_{FH} cell responses could enhance MCC control are not well understood. High affinity secreted antibodies could bind to T-Ag released from MCC as well as Fc receptors on the surface of antigen presenting cells, which can efficiently internalize T-Ag and present peptides bound to HLA class I to enhance MCC killing T-Ag-specific CD8+ T cells. Recent work in a mouse model of lung adenocarcinoma has also demonstrated that tumor antigen-specific B cell/T_{FH} responses can promote tumor killing responses of CD8+ T cells through the production of IL-21. Cui et al., Cell 2021; 184(25):6101-18 e13. This experimental example describes associations between CD4⁺ T cell phenotype and MCC progression and evidence of interactions between T cells and T-Ag-specific B cells.

Building upon previous work (lyer et al., Clin Cancer Res 2011; 17(21):6671-80), a strong tumor-infiltrating T-Ag-specific CD4+ T cell response in VP-MCC was identified (Longino et al., Cancer immunology research 2019; 7(10):1727-39). T-Ag-specific CD4⁺ T cells were identified using peptide stimulation assays and confirmed using peptide:HLA class II tetramers (lyer et al., supra; Longino et al., supra). These studies identified amino acids 209-228 of Large T-Ag as an immunodominant region containing shorter peptides presented by the HLA class II alleles, DRB1*04:01, DRB1*03:01, and DQB1*03:01 (Longino et al., supra). Other CD4⁺ T cell epitopes include large T-Ag₁₈₅₋₂₁₂, small T-Ag₁₂₁₋₁₄₀, and common T-Ag_{9-21, 25-44, & 49-51}.

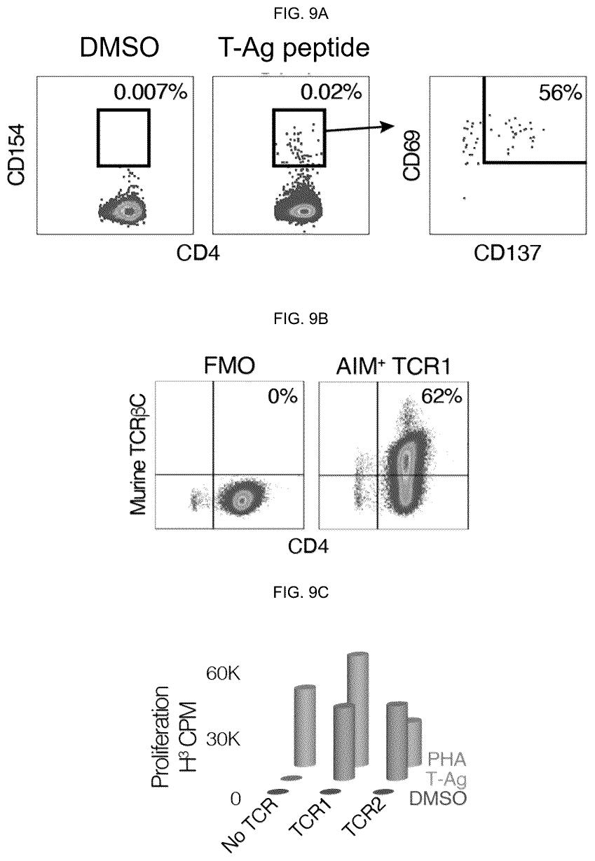

TABLE 1
Samples assessed in FIGS. 9A-9C

| Sample Type | Post-analysis outcome | Immunotherapy post-analysis? |
|---|---|---|
| Skin tumor | Non-progression | No |
| Infiltrated LN | Non-progression | No |
| Skin tumor | Progression | No |
| Infiltrated LN | Progression | No |
| PBMC | Non-progression | No |
| PBMC | Progression | No |
| Skin tumor | Non-progression | Yes |
| Infiltrated LN | Non-progression | Yes |
| Skin tumor | Progression | Yes |
| Infiltrated LN | Progression | Yes |
| PBMC | Non-progression | Yes |
| PBMC | Progression | Yes |

Assessing samples for the presence of T-Ag-specific T_{FH} cells is complicated by the requirement for T-Ag peptides to be presented to C04⁺ T cells bound to HLA class II molecules, which are remarkably heterogeneous in the human population. In fact, of the sixteen samples assessed in FIGS. 7A-7E, only four have DRB1*04:01, DRB1*03:01, or DQB1*03:01. Given this, the activation induced marker (AIM) assay (Dan et al., J Immunol 2016; 197(3):983-93; Havenar-Daughton et al., J Immunol 2016; 197(3):994-1002; Poloni et al., Immunol Cell Biol 2023; 101(6):491-503) was used to identify T-Ag-specific CD45RA-CXCR5+PD-1+T_{FH} and CD45RA-CXCR5-non-T_{FH} as CD4⁺ T cells responding to T-Ag peptide pools for the same sample set (Table 1). The expression of CXCL13 and ICOS will also be assessed in these experiments as these markers are expressed on non-canonical tumor-infiltrating CXCR5-TFH cells in other cancers (Gu-Trantien et al., JCI insight 2017; 2(11); Li et al., J Immunother Cancer 2021; 9(7)). In this experimental example, PBMCs from a DRB1*0801+VP-MCC patient were stimulated with common T-Ag₁₋₁₃ (MDLVLNRKEREAL) peptide for 18 hours prior to analysis of CD154, CD69 and CD137 expression by CD4⁺ T cells. In the presence of peptide, CD4⁺ T cells co-expressing CD154, CD69, and CD137 are present (FIG. 9A). CD154⁺CD69⁺CD137⁺CD4⁺ T cells were subsequently FACS-purified and TCRs from single cells were sequenced and expressed in autologous CD4⁺ reporter T cells using a lentivirus (FIG. 9B). Incubation of TCR-transduced CD4⁺ T cells with irradiated autologous PBMCs used as a source of antigen-presenting cells, pulsed with common T-Ag₁₁₃ peptide resulted in robust proliferation (FIG. 9C), confirming T-Ag-specificity of TCRs identified by the AIM assay. These approaches will be used to identify the T-Ag peptide:HLA Class II specificity of each cloned TCR.

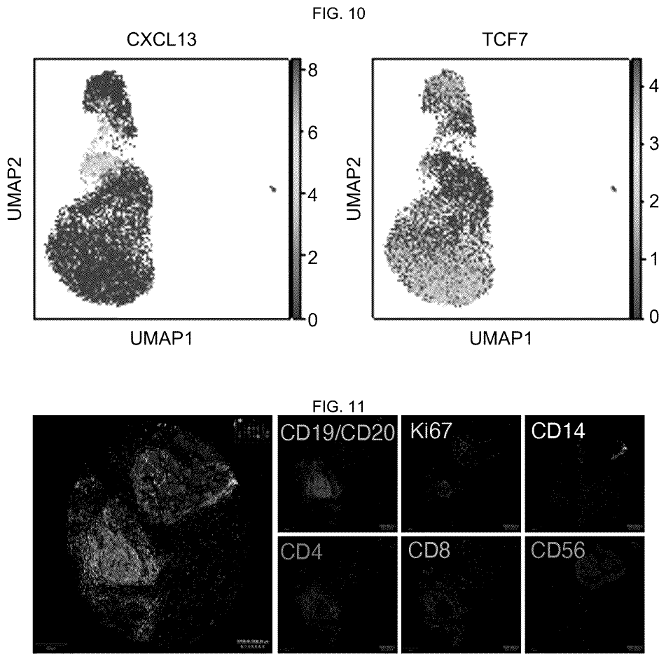

TCRs, mRNA expression, and surface phenotype from CD4⁺ T cells was evaluated alongside of B cells using 10×RNA sequencing. TFH cells can be identified in melanoma tumor samples as CD4+ T cells co-expressing CXCL13 and TCF7 (Veatch et al., Cancer Cell 2022; 40(4):393-409 e9). In this experimental example, a low frequency of CXCL13+TCF7+CD4+ is found in MCC tumor samples (FIG. 10), which is not surprising considering the low frequency of B cells in these samples (FIG. 6C). T-Ag-specific B cells comprise under 10% of B cells in these tumor samples,

Previous assessment of VP-MCC tumor sections did not reveal an association between tertiary lymphoid structures and disease progression (Nakamura et al., Front Oncol 2022; 12:811586). This experimental example describes profiling of cell localization in tumor sections using Akoya's PhenoCycler platform for highly multiplexed marker immunohistochemistry (Phillips et al., Nat Commun 2021; 12(1):6726) to identify tumor cells, and various subsets of T cells, B cells, dendritic cells, and macrophages. A 41-marker panel has already been validated by the Immunopathology Core (FIG. 11), and additional markers such as CD38, CD71 are being explored for deeper B cell analyses.

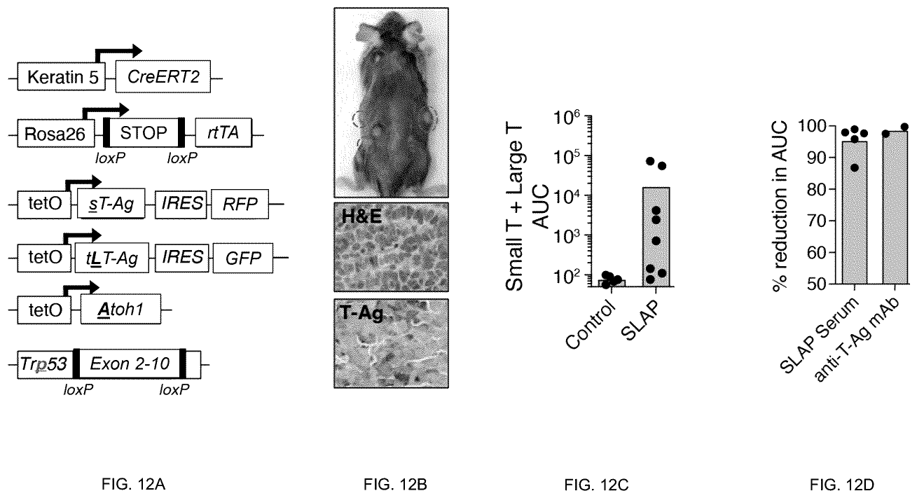

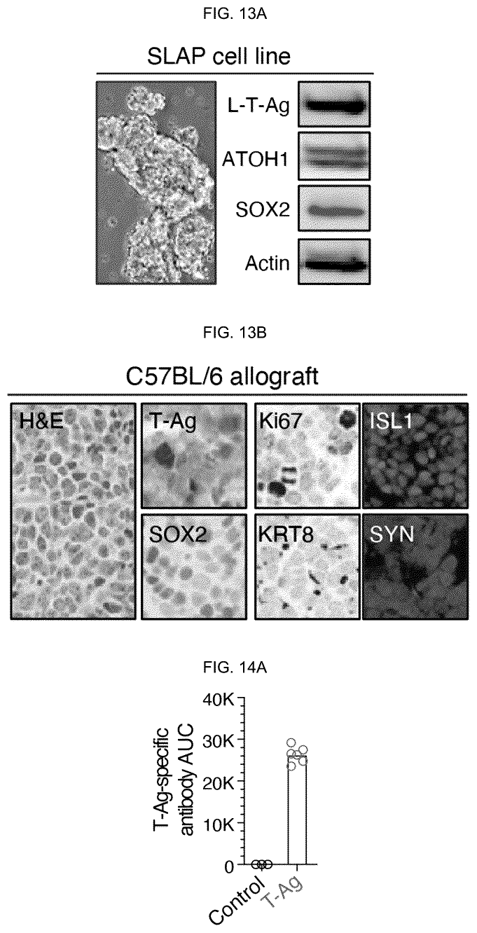

Interrogating immune mechanisms of tumor control using MCC mouse models. To explore B cell:T cell interactions, the newly developed SLAP mouse MCC model was used (Verhaegen et al., J Clin Invest 2022; 132(7)). This model can be induced to produce a T-Ag-driven VP-MCC-like disease. SLAP mice produce MCC tumors in response to tamoxifen and doxycycline by inducing the expression of the small and truncated large T-Ag, the Merkel cell-driving transcription factor Atoh1, and the inactivation of Trp53 in K5-expressing epidermal cells (FIGS. 12A, 12B) (Verhaegen et al., supra). Highly aggressive skin tumors with high similarity to human MCC are detectable in SLAP mice 11-22 weeks after transgene induction (Verhaegen et al., supra). Importantly, 5/8 animals with detectable tumors also produced T-Ag-specific serum antibodies with highly variable levels mirroring human MCC (FIG. 12C). Also mirroring human MCC, 87-99% of T-Ag-specific antibodies were specific for the region of T-Ag common between the large and small T-Ags (FIG. 12D). Together, these results indicate that the SLAP model can be used to study T-Ag-specific immune responses.

Assess T-Ag-specific T and B cell responses in murine SLAP MCC models. The SLAP model has been refined. This model was initially generated on a mixed C57BL/6×SJL background, which complicates studies of T cell responses. Therefore, the model has just been fully backcrossed to C57BL/6 so that the full complement of immunological techniques can be used such as peptide:MHC tetramers. With fully C57BL/6 SLAP mice, SLAP MCC cell lines can be produced and transferred into syngeneic recipient animals. A major advantage to this approach is that recipient animals should develop MCC tumors within 2-4 weeks, compared to 11-22 weeks after induction in the intact SLAP mouse. A second advantage to a transfer approach is that tumor cells could be transplanted into any knockout or transgenic animal on a C57BL/6 background to fully explore immune mechanisms. A third advantage to the generation of MCC lines is that they can be screened for different levels of aggressiveness and cell infiltration, like other cancer models (Meeth et al., Pigment Cell Melanoma Res 2016; 29(5):590-7). Preliminary results indicate that SLAP MCC lines can be easily isolated and grow robustly in vitro as suspension clusters (FIG. 13A) like human MCC lines (Meeth et al., Pigment Cell Melanoma Res 2016; 29(5):590-7; Velasquez et al., Front Microbiol 2018; 9:713; Verhaegen et al., The Journal of investigative dermatology 2014; 134(8):2241-50; Rosen et al., Lab Invest 1987; 56(3):302-12; Shuda et al., Proceedings of the National Academy of Sciences of the United States of America 2008; 105(42):16272-7; Houben et al., The Journal of investigative dermatology 2012; 132(1):254-6; Houben et al., The Journal of investigative dermatology 2013; 133(10):2453-60; Fischer et al., International journal of cancer 2010; 126(9):2133-42; Guastafierro et al., J Virol Methods 2013; 187(1):6-14). Both SLAP MCC cell lines derived from mice backcrossed to C57BL/6 for 7 generations produced detectable tumors following transfer into all four C57BL/6 recipients for each SLAP MCC line despite not being fully congenic and therefore potentially susceptible enhanced rejection. Allograft tumors histologically resembled parental tumors and expressed VP-MCC markers ISL1, SOX2, Keratin 8 (KRT8), synaptophysin (SYN) express T-Ag, the proliferation marker Ki67, and T-Ag (FIG. 13B).

Once the SLAP model is validated as fully congenic based on single nucleotide polymorphism genotyping, 20 cell lines from SLAP MCC tumors will be established and characterized. Immune responses to up to 4 lines will be characterized 7-60 days after tumor cell injection. T-Ag antibody levels will be assessed in serum as described above, and T-Ag-specific B cells assessed in tumors, tumor-draining lymph nodes and spleen with fluorescent T-Ag tetramers using flow cytometry. B cell subtypes will be further assessed by examining the expression of intracellular Ig, CD38, and GL7 as shown in FIGS. 4A-4D, with the addition of IgM and IgD to assess class switching within each subpopulation. CD4+ and CD8+ cell responses in these samples will be assessed using ELISpot, AIM assays, or tetramers produced by the Fred Hutch Immune Monitoring Core using previously characterized peptides such as large T-Ag₁₉₋₂₇ (IAPNCYGNI (SEQ ID NO: 1315)) (Gomez et al., Cell Biosci 2012; 2(1):36; Gomez et al., Cell Biosci 2013; 3(1):29) bound to H-2k^{b} to assess CD8⁺ T cells, or large T-Ag₁₄₁₋₁₅₅ (SSSGYGSFSASQASD ((SEQ ID NO: 1316)) (Zeng et al., Vaccine 2012; 30(7):1322-9) bound to I-A^{b} to assess CD4⁺ T cells. The latter is predicted by NetMHCllpan4.0 (Reynisson et al., Nucleic acids research 2020; 48(W1):W449-W54) to bind well to I-A^{b} when the first amino acid is excluded, making it an excellent candidate for tetramer production.

A T-Ag self-amplifying mRNA (samRNA) vaccine was recently advanced to clinical trials (NCT05422781) to determine whether vaccination after MCC treatment can prevent MCC progression. In this experimental example, it was assessed whether T-Ag vaccination can prevent or slow the progression of SLAP MCC. C57BL/6 mice will be subcutaneously injected with 10 μg of T-Ag samRNA or control samRNA vaccine at days 0, 14 and 28, followed by intradermal SLAP MCC cell line injection at day 35. A second cohort of mice will receive four weekly intradermal injections of 40 μg of T-Ag DNA or control DNA vaccine followed by electroporation, followed by intradermal SLAP MCC cell line injection at day 35. The T-Ag DNA vaccine is based on large T-Ag with a S220A mutation to decrease Rb binding (Schrama et al., International journal of cancer 2016; 138(5):1153-62).

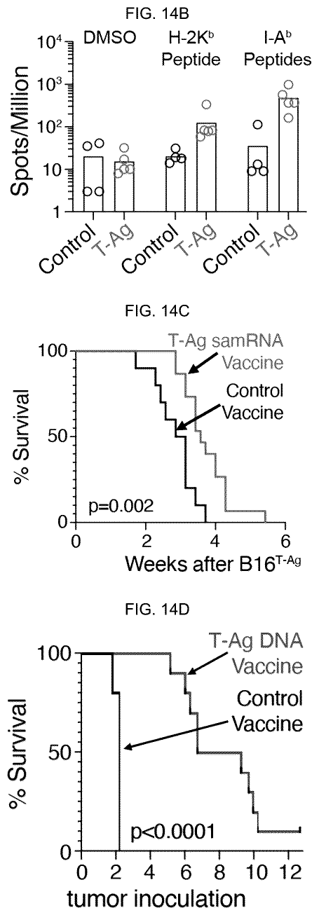

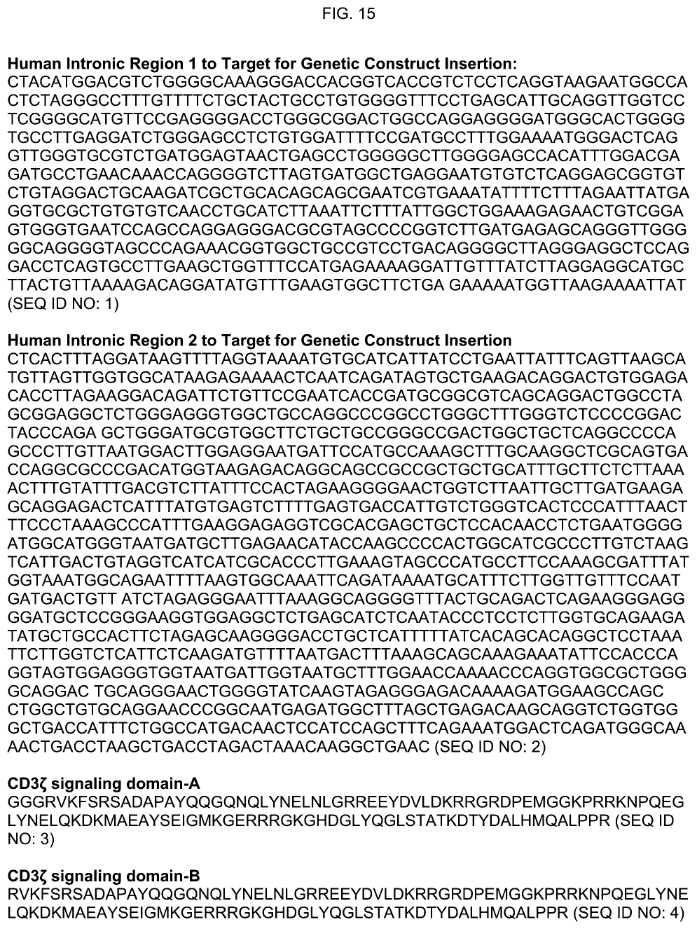

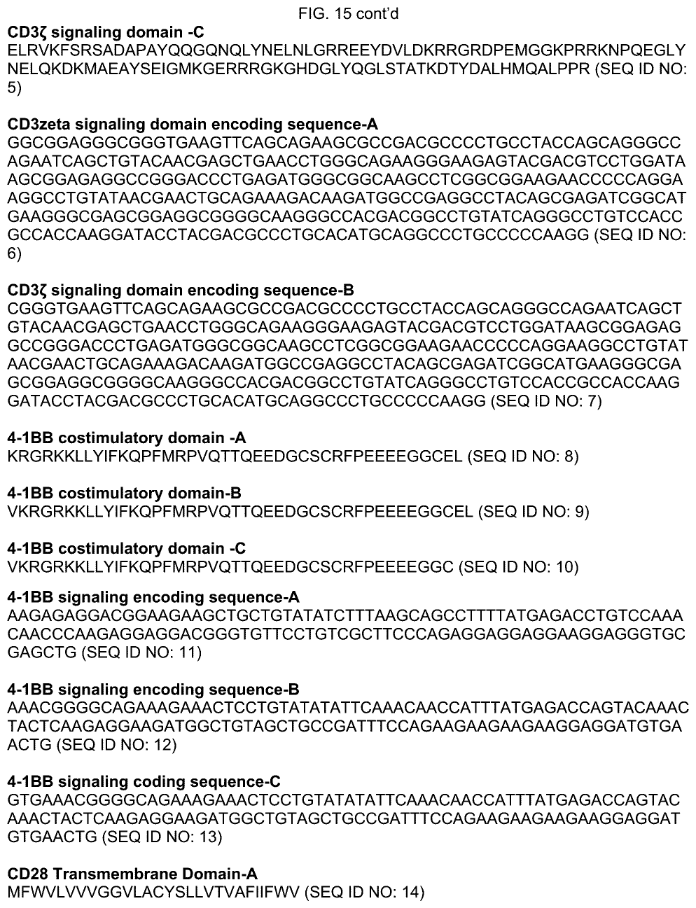

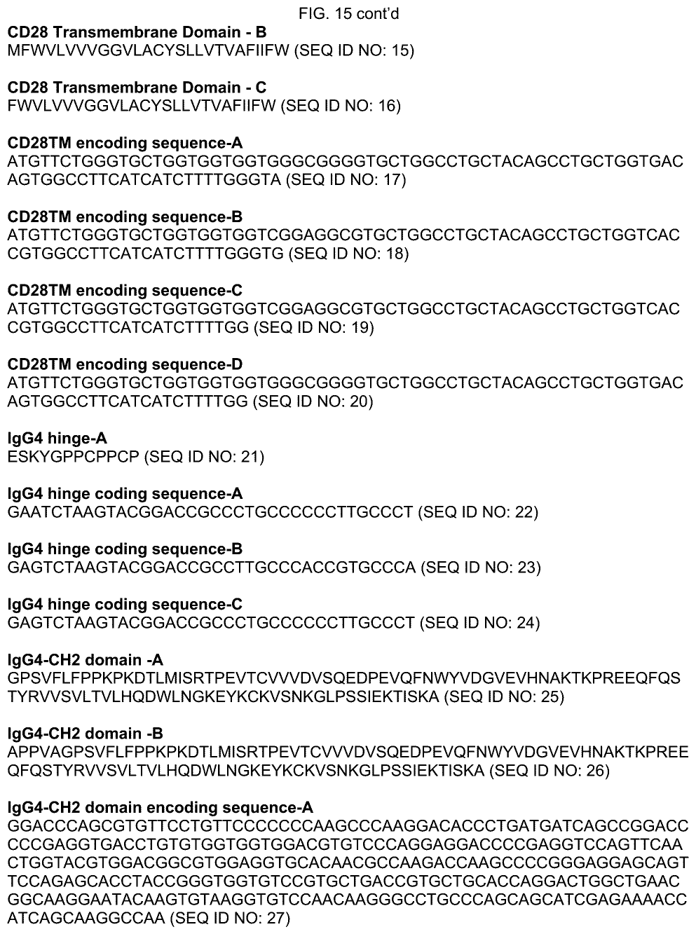

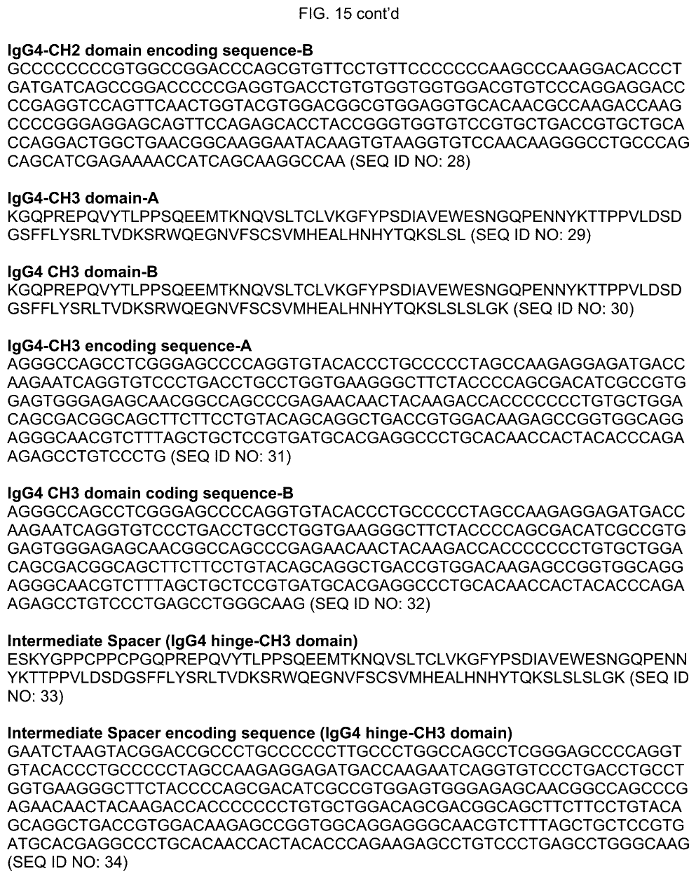

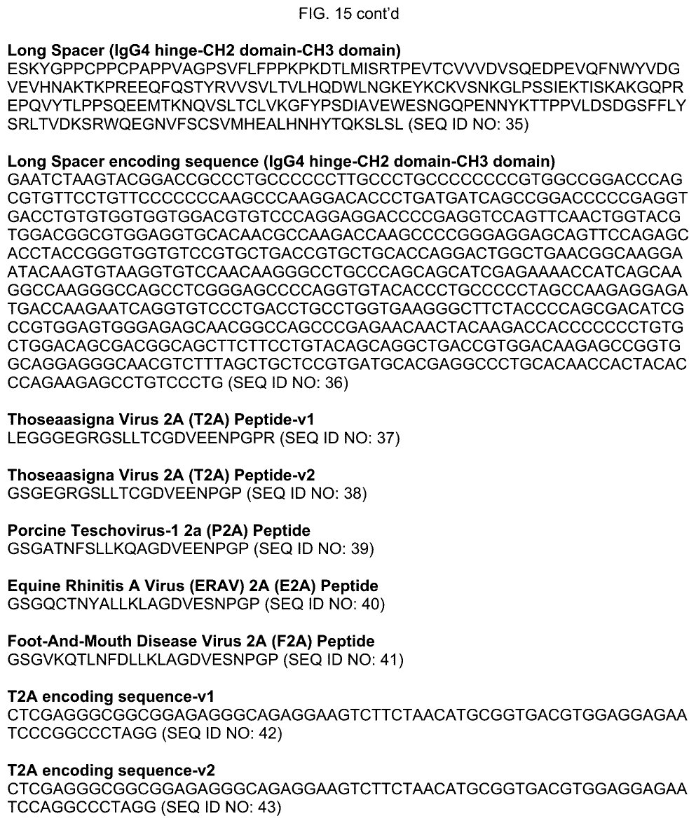

To assess therapeutic vaccination, SLAP MCC cell lines will be injected 14 days prior to 10 μg of T-Ag samRNA or control samRNA or DNA vaccine. In mice, this vaccine stimulates a potent T and B cell response measured by T-Ag-specific serum antibodies and T cell activation (FIGS. 14A, 14B). Tumor size will be monitored along with serum antibody levels and mice sacrificed if tumor grows to maximum allowable volume, or after 5 months if no evidence of tumor is detected. If tumors are present, tumor and tumor-draining lymph nodes will be assessed for the presence of T-Ag-specific B and T cells as described above.

In a proof-of-principle experiment, C57BL/6 mice were injected with T-Ag samRNA or control samRNA vaccine and challenged with a B16F10 melanoma cell line expressing large T-Ag. The B16^{T-Ag} melanoma line grows aggressively, and control mice need to be euthanized 2-4 weeks after tumor challenge (FIG. 14C). In contrast, T-Ag mRNA vaccinated mice exhibited delayed B16^{T-Ag} tumor cell growth, extending mouse survival to 3-5 weeks (FIG. 14C). As an alternative approach, it was found that a T-Ag DNA vaccine extended survival beyond 6 weeks for most mice (FIG. 14D) (Dy et al., Cancer research 2022; 82(12_Supplement):CT008-CT08).

Determine the contributions of antibodies, B cells, and T cells in control of optimized SLAP MCC. To probe the role that antibodies play in MCC, 15 mg/kg of T-Ag-specific antibody 1G04 with murine IgG1 and kappa constant regions or an irrelevant control murine antibody will be injected intraperitoneally 1 day prior to the SLAP MCC cell line injection. 1G04 was characterized in initial studies and binds T-Ag strongly (FIG. 5D).

(xx) Closing Paragraphs. The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al., Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (lie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed) Oxford University Press, N Y (1988); Biocomputing: Informatics and Genome Projects (Smith, D. W., ed) Academic Press, N Y (1994); Computer Analysis of Sequence Data, Part I (Griffin, A. M., and Griffin, H. G., eds) Humana Press, N J (1994); Sequence Analysis in Molecular Biology (Von Heijne, G., ed) Academic Press (1987); and Sequence Analysis Primer (Gribskov, M. and Devereux, J., eds) Oxford University Press, NY (1992). Methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridize under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g., 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding domain (of, for example, an antibody binding domain to its antigen) to its cognate binding molecule with an affinity or Kₐ (i.e., an equilibrium association constant of a particular binding interaction with units of 1/M) equal to or greater than 10⁵ M⁻¹, while not significantly associating with any other molecules or components in a relevant environment sample. Binding domains may be classified as “high affinity” or “low affinity”. In particular embodiments, “high affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Kₐ (equilibrium association constant) for the cognate binding molecule that is higher than the reference binding domain or due to a K_{d} (dissociation constant) for the cognate binding molecule that is less than that of the reference binding domain, or due to an off-rate (K_{off}) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al., Molecular Cloning: A Laboratory Manual, 2nd Edition (1989); F. M. Ausubel, et al., eds., Current Protocols in Molecular Biology, (1987); the series Methods IN Enzymology (Academic Press, Inc); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant increase in T antigen-expressing cells, as described herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; 19% of the stated value; ±18% of the stated value; 17% of the stated value; 16% of the stated value; ±15% of the stated value; 14% of the stated value; ±13% of the stated value; 12% of the stated value; 11% of the stated value; 10% of the stated value; 9% of the stated value; 8% of the stated value; 7% of the stated value; ±6% of the stated value; 5% of the stated value; 4% of the stated value; ±3% of the stated value; 2% of the stated value; or +1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

1-202. (canceled)

203. An antibody or fragment thereof that binds a T antigen of the Merkel cell polyomavirus comprising a binding domain of antibody 1C10, 3B09, 1B09, 1G04, 2H04, W056-03, W056-04, W056-05, W056-06, W056-08, W056-09, W056-10, Z156-02, Z156-03, Z249-01, Z249-02, Z249-03, Z249-04, Z249-05, Z249-06, Z308-03, Z308-04, Z308-05, Z504-03, Z504-04, Z504-06, Z504-07, Z513-01, Z513-02, Z538-03, Z573-03, Z582-03, Z582-14, Z582-15, Z082-U1, Z082-U5, Z082-U6, W056-01, W056-02, W917-01, W917-02, W917-03, Z504-01, Z504-02, Z538-01, Z538-02, Z573-01, or Z573-02 according to North, Kabat, IMGT, Chothia, or Contact;
wherein 1C10 comprises a heavy chain variable region comprising a complementarity determining region (CDR) heavy (H)1, CDRH2, and CDRH3 and a light chain variable region comprising a CDR light (L)1, CDRL2, and CDRL3;
wherein the CDRH1 comprises the sequence as set forth in SEQ ID NO: 57, the CDRH2 comprises the sequence as set forth in SEQ ID NO: 58, the CDRH3 comprises the sequence as set forth in SEQ ID NO: 59, the CDRL1 comprises the sequence as set forth in SEQ ID NO: 60, the CDRL2 comprises the sequence as set forth in SEQ ID NO: 61, and the CDRL3 comprises the sequence as set forth in SEQ ID NO: 62, according to North;
wherein the CDRH1 comprises the sequence as set forth in SEQ ID NO: 63, the CDRH2 comprises the sequence as set forth in SEQ ID NO: 64, the CDRH3 comprises the sequence as set forth in SEQ ID NO: 65, the CDRL1 comprises the sequence as set forth in SEQ ID NO: 60, the CDRL2 comprises the sequence as set forth in SEQ ID NO: 66, and the CDRL3 comprises the sequence as set forth in SEQ ID NO: 62, according to Kabat;
wherein the CDRH1 comprises the sequence as set forth in SEQ ID NO: 67, the CDRH2 comprises the sequence as set forth in SEQ ID NO: 68, the CDRH3 comprises the sequence as set forth in SEQ ID NO: 59, the CDRL1 comprises the sequence as set forth in SEQ ID NO: 69, the CDRL2 comprises the sequence KDT, and the CDRL3 comprises the sequence as set forth in SEQ ID NO: 62, according to IMGT;
wherein the CDRH1 comprises the sequence as set forth in SEQ ID NO: 70, the CDRH2 comprises the sequence as set forth in SEQ ID NO: 71, the CDRH3 comprises the sequence as set forth in SEQ ID NO: 65, the CDRL1 comprises the sequence as set forth in SEQ ID NO: 60, the CDRL2 comprises the sequence as set forth in SEQ ID NO: 66, and the CDRL3 comprises the sequence as set forth in SEQ ID NO: 62, according to Chothia; or
wherein the CDRH1 comprises the sequence as set forth in SEQ ID NO: 72, the CDRH2 comprises the sequence as set forth in SEQ ID NO: 71, the CDRH3 comprises the sequence as set forth in SEQ ID NO: 73, the CDRL1 comprises the sequence as set forth in SEQ ID NO: 74, the CDRL2 comprises the sequence as set forth in SEQ ID NO: 75, and the CDRL3 comprises the sequence as set forth in SEQ ID NO: 76, according to Contact;
wherein 3B09 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 79, the CDRH2 comprises the sequence as set forth in SEQ ID NO: 80, the CDRH3 comprises the sequence as set forth in SEQ ID NO: 81, the CDRL1 comprises the sequence as set forth in SEQ ID NO: 82, the CDRL2 comprises the sequence as set forth in SEQ ID NO: 83, the CDRL3 comprises the sequence as set forth in SEQ ID NO: 84, according to North;
wherein the CDRH1 comprises the sequence as set forth in SEQ ID NO: 85, the CDRH2 comprises the sequence as set forth in SEQ ID NO: 86, the CDRH3 comprises the sequence as set forth in SEQ ID NO: 87, the CDRL1 comprises the sequence as set forth in SEQ ID NO: 82, the CDRL2 comprises the sequence as set forth in SEQ ID NO: 88, the CDRL3 comprises the sequence as set forth in SEQ ID NO: 84, according to Kabat;
wherein the CDRH1 comprises the sequence as set forth in SEQ ID NO: 89, the CDRH2 comprises the sequence as set forth in SEQ ID NO: 90, the CDRH3 comprises the sequence as set forth in SEQ ID NO: 81, the CDRL1 comprises the sequence as set forth in SEQ ID NO: 91, the CDRL2 comprises the sequence DVH, the CDRL3 comprises the sequence as set forth in SEQ ID NO: 84, according to IMGT;
wherein the CDRH1 comprises the sequence as set forth in SEQ ID NO: 92, the CDRH2 comprises the sequence as set forth in SEQ ID NO: 93, the CDRH3 comprises the sequence as set forth in SEQ ID NO: 87, the CDRL1 comprises the sequence as set forth in SEQ ID NO: 82, the CDRL2 comprises the sequence as set forth in SEQ ID NO: 88, the CDRL3 comprises the sequence as set forth in SEQ ID NO: 84, according Chothia; or
wherein the CDRH1 comprises the sequence as set forth in SEQ ID NO: 94, the CDRH2 comprises the sequence as set forth in SEQ ID NO: 95, the CDRH3 comprises the sequence as set forth in SEQ ID NO: 96, the CDRL1 comprises the sequence as set forth in SEQ ID NO: 97, the CDRL2 comprises the sequence as set forth in SEQ ID NO: 98, the CDRL3 comprises the sequence as set forth in SEQ ID NO: 99, according to Contact;
wherein 1B09 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 102, the CDRH2 comprises the sequence as set forth in SEQ ID NO: 103, the CDRH3 comprises the sequence as set forth in SEQ ID NO: 104, the CDRL1 comprises the sequence as set forth in SEQ ID NO: 105, the CDRL2 comprises the sequence as set forth in SEQ ID NO: 106, the CDRL3 comprises the sequence as set forth in SEQ ID NO: 107, according to North;
wherein the CDRH1 comprises the sequence as set forth in SEQ ID NO: 108, the CDRH2 comprises the sequence as set forth in SEQ ID NO: 109, the CDRH3 comprises the sequence as set forth in SEQ ID NO: 110, the CDRL1 comprises the sequence as set forth in SEQ ID NO: 105, the CDRL2 comprises the sequence as set forth in SEQ ID NO: 111, the CDRL3 comprises the sequence as set forth in SEQ ID NO: 107, according to Kabat;
wherein the CDRH1 comprises the sequence as set forth in SEQ ID NO: 112, the CDRH2 comprises the sequence as set forth in SEQ ID NO: 113, the CDRH3 comprises the sequence as set forth in SEQ ID NO: 104, the CDRL1 comprises the sequence as set forth in SEQ ID NO: 114, the CDRL2 comprises the sequence DVS, the CDRL3 comprises the sequence as set forth in SEQ ID NO: 107, according to IMGT;
wherein the CDRH1 comprises the sequence as set forth in SEQ ID NO: 115, the CDRH2 comprises the sequence as set forth in SEQ ID NO: 116, the CDRH3 comprises the sequence as set forth in SEQ ID NO: 110, the CDRL1 comprises the sequence as set forth in SEQ ID NO: 105, the CDRL2 comprises the sequence as set forth in SEQ ID NO: 111, the CDRL3 comprises the sequence as set forth in SEQ ID NO: 107, according to Chothia; or
wherein the CDRH1 comprises the sequence as set forth in SEQ ID NO: 117, the CDRH2 comprises the sequence as set forth in SEQ ID NO: 118, the CDRH3 comprises the sequence as set forth in SEQ ID NO: 119, the CDRL1 comprises the sequence as set forth in SEQ ID NO: 120, the CDRL2 comprises the sequence as set forth in SEQ ID NO: 121, the CDRL3 comprises the sequence as set forth in SEQ ID NO: 122, according to Contact;
wherein 1G04 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 125, the CDRH2 comprises the sequence as set forth in SEQ ID NO: 126, the CDRH3 comprises the sequence as set forth in SEQ ID NO: 127, the CDRL1 comprises the sequence as set forth in SEQ ID NO: 128, according to North;
wherein the CDRL2 comprises the sequence as set forth in SEQ ID NO: 129, the CDRL3 comprises the sequence as set forth in SEQ ID NO: 130, the CDRH1 comprises the sequence as set forth in SEQ ID NO: 131, the CDRH2 comprises the sequence as set forth in SEQ ID NO: 132, the CDRH3 comprises the sequence as set forth in SEQ ID NO: 133, the CDRL1 comprises the sequence as set forth in SEQ ID NO: 128, the CDRL2 comprises the sequence as set forth in SEQ ID NO: 134, the CDRL3 comprises the sequence as set forth in SEQ ID NO: 130, according to Kabat;
wherein the CDRH1 comprises the sequence as set forth in SEQ ID NO: 135, the CDRH2 comprises the sequence as set forth in SEQ ID NO: 136, the CDRH3 comprises the sequence as set forth in SEQ ID NO: 127, the CDRL1 comprises the sequence as set forth in SEQ ID NO: 137, the CDRL2 comprises the sequence LAS, the CDRL3 comprises the sequence as set forth in SEQ ID NO: 130, according to IMGT;
wherein the CDRH1 comprises the sequence as set forth in SEQ ID NO: 138, the CDRH2 comprises the sequence as set forth in SEQ ID NO: 139, the CDRH3 comprises the sequence as set forth in SEQ ID NO: 133, the CDRL1 comprises the sequence as set forth in SEQ ID NO: 128, the CDRL2 comprises the sequence as set forth in SEQ ID NO: 134, the CDRL3 comprises the sequence as set forth in SEQ ID NO: 130, according to Chothia; or
wherein the CDRH1 comprises the sequence as set forth in SEQ ID NO: 140, the CDRH2 comprises the sequence as set forth in SEQ ID NO: 141, the CDRH3 comprises the sequence as set forth in SEQ ID NO: 142, the CDRL1 comprises the sequence as set forth in SEQ ID NO: 143, the CDRL2 comprises the sequence as set forth in SEQ ID NO: 144, the CDRL3 comprises the sequence as set forth in SEQ ID NO: 145, according to Contact;
wherein 2H04 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 148, the CDRH2 comprises the sequence as set forth in SEQ ID NO: 149, the CDRH3 comprises the sequence as set forth in SEQ ID NO: 150, the CDRL1 comprises the sequence as set forth in SEQ ID NO: 151, the CDRL2 comprises the sequence as set forth in SEQ ID NO: 152, the CDRL3 comprises the sequence as set forth in SEQ ID NO: 153, according to North;
wherein the CDRH1 comprises the sequence as set forth in SEQ ID NO: 154, the CDRH2 comprises the sequence as set forth in SEQ ID NO: 155, the CDRH3 comprises the sequence as set forth in SEQ ID NO: 156, the CDRL1 comprises the sequence as set forth in SEQ ID NO: 151, the CDRL2 comprises the sequence as set forth in SEQ ID NO: 157, the CDRL3 comprises the sequence as set forth in SEQ ID NO: 153, according to Kabat;
wherein the CDRH1 comprises the sequence as set forth in SEQ ID NO: 158, the CDRH2 comprises the sequence as set forth in SEQ ID NO: 159, the CDRH3 comprises the sequence as set forth in SEQ ID NO: 150, the CDRL1 comprises the sequence as set forth in SEQ ID NO: 160, the CDRL2 comprises the sequence DDR, the CDRL3 comprises the sequence as set forth in SEQ ID NO: 153, according to IMGT;
wherein the CDRH1 comprises the sequence as set forth in SEQ ID NO: 161, the CDRH2 comprises the sequence as set forth in SEQ ID NO: 162, the CDRH3 comprises the sequence as set forth in SEQ ID NO: 156, the CDRL1 comprises the sequence as set forth in SEQ ID NO: 151, the CDRL2 comprises the sequence as set forth in SEQ ID NO: 157, the CDRL3 comprises the sequence as set forth in SEQ ID NO: 153, according to Chothia; or
wherein the CDRH1 comprises the sequence as set forth in SEQ ID NO: 163, the CDRH2 comprises the sequence as set forth in SEQ ID NO: 164, the CDRH3 comprises the sequence as set forth in SEQ ID NO: 165, the CDRL1 comprises the sequence as set forth in SEQ ID NO: 166, the CDRL2 comprises the sequence as set forth in SEQ ID NO: 167, the CDRL3 comprises the sequence as set forth in SEQ ID NO: 168, according to Contact;
wherein W056-03 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 250, the CDRH2 comprises the sequence as set forth in SEQ ID NO: 251, the CDRH3 comprises the sequence as set forth in SEQ ID NO: 252, the CDRL1 comprises the sequence as set forth in SEQ ID NO: 253, the CDRL2 comprises the sequence as set forth in SEQ ID NO: 254, and the CDRL3 comprises the sequence as set forth in SEQ ID NO: 255, according to North;
wherein the CDRH1 comprises the sequence as set forth in SEQ ID NO: 256, the CDRH2 comprises the sequence as set forth in SEQ ID NO: 257, the CDRH3 comprises the sequence as set forth in SEQ ID NO: 258, the CDRL1 comprises the sequence as set forth in SEQ ID NO: 253, the CDRL2 comprises the sequence as set forth in SEQ ID NO: 259, and the CDRL3 comprises the sequence as set forth in SEQ ID NO: 255, according to Kabat;
wherein the CDRH1 comprises the sequence as set forth in SEQ ID NO: 260, the CDRH2 comprises the sequence as set forth in SEQ ID NO: 261, the CDRH3 comprises the sequence as set forth in SEQ ID NO: 252, the CDRL1 comprises the sequence as set forth in SEQ ID NO: 262, the CDRL2 comprises the sequence GN, and the CDRL3 comprises the sequence as set forth in SEQ ID NO: 255, according to IMGT;
wherein the CDRH1 comprises the sequence as set forth in SEQ ID NO: 263, the CDRH2 comprises the sequence as set forth in SEQ ID NO: 264, the CDRH3 comprises the sequence as set forth in SEQ ID NO: 258, the CDRL1 comprises the sequence as set forth in SEQ ID NO: 253, the CDRL2 comprises the sequence as set forth in SEQ ID NO: 259, and the CDRL3 comprises the sequence as set forth in SEQ ID NO: 255, according to Chothia; or
wherein the CDRH1 comprises the sequence as set forth in SEQ ID NO: 265, the CDRH2 comprises the sequence as set forth in SEQ ID NO: 266, the CDRH3 comprises the sequence as set forth in SEQ ID NO: 267, the CDRL1 comprises the sequence as set forth in SEQ ID NO: 268, the CDRL2 comprises the sequence as set forth in SEQ ID NO: 269, and the CDRL3 comprises the sequence as set forth in SEQ ID NO: 270, according to Contact;
wherein W056-04 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 271, the CDRH2 comprises the sequence as set forth in SEQ ID NO: 272, the CDRH3 comprises the sequence as set forth in SEQ ID NO: 273, the CDRL1 comprises the sequence as set forth in SEQ ID NO: 274, the CDRL2 comprises the sequence as set forth in SEQ ID NO: 275, and the CDRL3 comprises the sequence as set forth in SEQ ID NO: 276, according to North;
wherein the CDRH1 comprises the sequence as set forth in SEQ ID NO: 277, the CDRH2 comprises the sequence as set forth in SEQ ID NO: 278, the CDRH3 comprises the sequence as set forth in SEQ ID NO: 279, the CDRL1 comprises the sequence as set forth in SEQ ID NO: 274, the CDRL2 comprises the sequence as set forth in SEQ ID NO: 280, and the CDRL3 comprises the sequence as set forth in SEQ ID NO: 276, according to Kabat;
wherein the CDRH1 comprises the sequence as set forth in SEQ ID NO: 281, the CDRH2 comprises the sequence as set forth in SEQ ID NO: 282, the CDRH3 comprises the sequence as set forth in SEQ ID NO: 273, the CDRL1 comprises the sequence as set forth in SEQ ID NO: 283, the CDRL2 comprises the sequence EV, and the CDRL3 comprises the sequence as set forth in SEQ ID NO: 276, according to IMGT;
wherein the CDRH1 comprises the sequence as set forth in SEQ ID NO: 284 the CDRH2 comprises the sequence as set forth in SEQ ID NO: 285, the CDRH3 comprises the sequence as set forth in SEQ ID NO: 279, the CDRL1 comprises the sequence as set forth in SEQ ID NO: 274, the CDRL2 comprises the sequence as set forth in SEQ ID NO: 280, and the CDRL3 comprises the sequence as set forth in SEQ ID NO: 276, according to Chothia; or
wherein the CDRH1 comprises the sequence as set forth in SEQ ID NO: 286, the CDRH2 comprises the sequence as set forth in SEQ ID NO: 287, the CDRH3 comprises the sequence as set forth in SEQ ID NO: 288, the CDRL1 comprises the sequence as set forth in SEQ ID NO: 289, the CDRL2 comprises the sequence as set forth in SEQ ID NO: 290, and the CDRL3 comprises the sequence as set forth in SEQ ID NO: 291, according to Contact;
wherein W056-05 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 292, the CDRH2 comprises the sequence as set forth in SEQ ID NO: 293, the CDRH3 comprises the sequence as set forth in SEQ ID NO: 294, the CDRL1 comprises the sequence as set forth in SEQ ID NO: 295, the CDRL2 comprises the sequence as set forth in SEQ ID NO: 296, and the CDRL3 comprises the sequence as set forth in SEQ ID NO: 297, according to North;
wherein the CDRH1 comprises the sequence as set forth in SEQ ID NO: 298, the CDRH2 comprises the sequence as set forth in SEQ ID NO: 299, the CDRH3 comprises the sequence as set forth in SEQ ID NO: 300, the CDRL1 comprises the sequence as set forth in SEQ ID NO: 295, the CDRL2 comprises the sequence as set forth in SEQ ID NO: 301, and the CDRL3 comprises the sequence as set forth in SEQ ID NO: 297, according to Kabat;
wherein the CDRH1 comprises the sequence as set forth in SEQ ID NO: 302, the CDRH2 comprises the sequence as set forth in SEQ ID NO: 303, the CDRH3 comprises the sequence as set forth in SEQ ID NO: 294, the CDRL1 comprises the sequence as set forth in SEQ ID NO: 304, the CDRL2 comprises the sequence AA, and the CDRL3 comprises the sequence as set forth in SEQ ID NO: 297, according to IMGT;
wherein the CDRH1 comprises the sequence as set forth in SEQ ID NO: 305 the CDRH2 comprises the sequence as set forth in SEQ ID NO: 306, the CDRH3 comprises the sequence as set forth in SEQ ID NO: 300, the CDRL1 comprises the sequence as set forth in SEQ ID NO: 295, the CDRL2 comprises the sequence as set forth in SEQ ID NO: 301, and the CDRL3 comprises the sequence as set forth in SEQ ID NO: 297, according to Chothia; or
wherein the CDRH1 comprises the sequence as set forth in SEQ ID NO: 307, the CDRH2 comprises the sequence as set forth in SEQ ID NO: 308, the CDRH3 comprises the sequence as set forth in SEQ ID NO: 309, the CDRL1 comprises the sequence as set forth in SEQ ID NO: 310, the CDRL2 comprises the sequence as set forth in SEQ ID NO: 311, and the CDRL3 comprises the sequence as set forth in SEQ ID NO: 312, according to Contact;
wherein W056-06 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 313, the CDRH2 comprises the sequence as set forth in SEQ ID NO: 314, the CDRH3 comprises the sequence as set forth in SEQ ID NO: 315, the CDRL1 comprises the sequence as set forth in SEQ ID NO: 316, the CDRL2 comprises the sequence as set forth in SEQ ID NO: 317, and the CDRL3 comprises the sequence as set forth in SEQ ID NO: 318, according to North;
wherein the CDRH1 comprises the sequence as set forth in SEQ ID NO: 319, the CDRH2 comprises the sequence as set forth in SEQ ID NO: 320, the CDRH3 comprises the sequence as set forth in SEQ ID NO: 321, the CDRL1 comprises the sequence as set forth in SEQ ID NO: 316, the CDRL2 comprises the sequence as set forth in SEQ ID NO: 322, and the CDRL3 comprises the sequence as set forth in SEQ ID NO: 318, according to Kabat;
wherein the CDRH1 comprises the sequence as set forth in SEQ ID NO: 323, the CDRH2 comprises the sequence as set forth in SEQ ID NO: 324, the CDRH3 comprises the sequence as set forth in SEQ ID NO: 315, the CDRL1 comprises the sequence as set forth in SEQ ID NO: 325, the CDRL2 comprises the sequence EA, and the CDRL3 comprises the sequence as set forth in SEQ ID NO: 318, according to IMGT;
wherein the CDRH1 comprises the sequence as set forth in SEQ ID NO: 326, the CDRH2 comprises the sequence as set forth in SEQ ID NO: 327, the CDRH3 comprises the sequence as set forth in SEQ ID NO: 321, the CDRL1 comprises the sequence as set forth in SEQ ID NO: 316, the CDRL2 comprises the sequence as set forth in SEQ ID NO: 322, and the CDRL3 comprises the sequence as set forth in SEQ ID NO: 318, according to Chothia; or
wherein the CDRH1 comprises the sequence as set forth in SEQ ID NO: 323, the CDRH2 comprises the sequence as set forth in SEQ ID NO: 324, the CDRH3 comprises the sequence as set forth in SEQ ID NO: 325, the CDRL1 comprises the sequence as set forth in SEQ ID NO: 326, the CDRL2 comprises the sequence as set forth in SEQ ID NO: 327, and the CDRL3 comprises the sequence as set forth in SEQ ID NO: 335, according to Contact;
wherein W056-08 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 271, the CDRH2 comprises the sequence as set forth in SEQ ID NO: 272, the CDRH3 comprises the sequence as set forth in SEQ ID NO: 273, the CDRL1 comprises the sequence as set forth in SEQ ID NO: 274, the CDRL2 comprises the sequence as set forth in SEQ ID NO: 275, and the CDRL3 comprises the sequence as set forth in SEQ ID NO: 276, according to North;
wherein the CDRH1 comprises the sequence as set forth in SEQ ID NO: 277, the CDRH2 comprises the sequence as set forth in SEQ ID NO: 278, the CDRH3 comprises the sequence as set forth in SEQ ID NO: 279, the CDRL1 comprises the sequence as set forth in SEQ ID NO: 274, the CDRL2 comprises the sequence as set forth in SEQ ID NO: 280, and the CDRL3 comprises the sequence as set forth in SEQ ID NO: 276, according to Kabat;
wherein the CDRH1 comprises the sequence as set forth in SEQ ID NO: 281, the CDRH2 comprises the sequence as set forth in SEQ ID NO: 282, the CDRH3 comprises the sequence as set forth in SEQ ID NO: 273, the CDRL1 comprises the sequence as set forth in SEQ ID NO: 283, the CDRL2 comprises the sequence EV, and the CDRL3 comprises the sequence as set forth in SEQ ID NO: 318 according to IMGT;
wherein the CDRH1 comprises the sequence as set forth in SEQ ID NO: 284, the CDRH2 comprises the sequence as set forth in SEQ ID NO: 285, the CDRH3 comprises the sequence as set forth in SEQ ID NO: 279, the CDRL1 comprises the sequence as set forth in SEQ ID NO: 274, the CDRL2 comprises the sequence as set forth in SEQ ID NO: 280, and the CDRL3 comprises the sequence as set forth in SEQ ID NO: 276, according to Chothia; or
wherein the CDRH1 comprises the sequence as set forth in SEQ ID NO: 286, the CDRH2 comprises the sequence as set forth in SEQ ID NO: 287, the CDRH3 comprises the sequence as set forth in SEQ ID NO: 288, the CDRL1 comprises the sequence as set forth in SEQ ID NO: 289, the CDRL2 comprises the sequence as set forth in SEQ ID NO: 290, and the CDRL3 comprises the sequence as set forth in SEQ ID NO: 291, according to Contact;
wherein W056-09 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 328, the CDRH2 comprises the sequence as set forth in SEQ ID NO: 314, the CDRH3 comprises the sequence as set forth in SEQ ID NO: 315, the CDRL1 comprises the sequence as set forth in SEQ ID NO: 329, the CDRL2 comprises the sequence as set forth in SEQ ID NO: 317, and the CDRL3 comprises the sequence as set forth in SEQ ID NO: 318, according to North;
wherein the CDRH1 comprises the sequence as set forth in SEQ ID NO: 319, the CDRH2 comprises the sequence as set forth in SEQ ID NO: 320, the CDRH3 comprises the sequence as set forth in SEQ ID NO: 321, the CDRL1 comprises the sequence as set forth in SEQ ID NO: 329, the CDRL2 comprises the sequence as set forth in SEQ ID NO: 322, and the CDRL3 comprises the sequence as set forth in SEQ ID NO: 318, according to Kabat;
wherein the CDRH1 comprises the sequence as set forth in SEQ ID NO: 330, the CDRH2 comprises the sequence as set forth in SEQ ID NO: 324, the CDRH3 comprises the sequence as set forth in SEQ ID NO: 315, the CDRL1 comprises the sequence as set forth in SEQ ID NO: 331, the CDRL2 comprises the sequence EV, and the CDRL3 comprises the sequence as set forth in SEQ ID NO: 318 according to IMGT;
wherein the CDRH1 comprises the sequence as set forth in SEQ ID NO: 332, the CDRH2 comprises the sequence as set forth in SEQ ID NO: 327, the CDRH3 comprises the sequence as set forth in SEQ ID NO: 321, the CDRL1 comprises the sequence as set forth in SEQ ID NO: 329, the CDRL2 comprises the sequence as set forth in SEQ ID NO: 322, and the CDRL3 comprises the sequence as set forth in SEQ ID NO: 318, according to Chothia; or
wherein the CDRH1 comprises the sequence as set forth in SEQ ID NO: 333, the CDRH2 comprises the sequence as set forth in SEQ ID NO: 324, the CDRH3 comprises the sequence as set forth in SEQ ID NO: 325, the CDRL1 comprises the sequence as set forth in SEQ ID NO: 334, the CDRL2 comprises the sequence as set forth in SEQ ID NO: 327, and the CDRL3 comprises the sequence as set forth in SEQ ID NO: 335, according to Contact;
wherein W056-10 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 336, the CDRH2 comprises the sequence as set forth in SEQ ID NO: 337, the CDRH3 comprises the sequence as set forth in SEQ ID NO: 338, the CDRL1 comprises the sequence as set forth in SEQ ID NO: 339, the CDRL2 comprises the sequence as set forth in SEQ ID NO: 340, and the CDRL3 comprises the sequence as set forth in SEQ ID NO: 341, according to North;
wherein the CDRH1 comprises the sequence as set forth in SEQ ID NO: 342, the CDRH2 comprises the sequence as set forth in SEQ ID NO: 343, the CDRH3 comprises the sequence as set forth in SEQ ID NO: 344, the CDRL1 comprises the sequence as set forth in SEQ ID NO: 339, the CDRL2 comprises the sequence as set forth in SEQ ID NO: 345, and the CDRL3 comprises the sequence as set forth in SEQ ID NO: 341, according to Kabat;
wherein the CDRH1 comprises the sequence as set forth in SEQ ID NO: 346, the CDRH2 comprises the sequence as set forth in SEQ ID NO: 347, the CDRH3 comprises the sequence as set forth in SEQ ID NO: 338, the CDRL1 comprises the sequence as set forth in SEQ ID NO: 348, the CDRL2 comprises the sequence AA, and the CDRL3 comprises the sequence as set forth in SEQ ID NO: 341 according to IMGT;
wherein the CDRH1 comprises the sequence as set forth in SEQ ID NO: 349, the CDRH2 comprises the sequence as set forth in SEQ ID NO: 350, the CDRH3 comprises the sequence as set forth in SEQ ID NO: 344, the CDRL1 comprises the sequence as set forth in SEQ ID NO: 339, the CDRL2 comprises the sequence as set forth in SEQ ID NO: 345, and the CDRL3 comprises the sequence as set forth in SEQ ID NO: 341, according to Chothia; or
wherein the CDRH1 comprises the sequence as set forth in SEQ ID NO: 351, the CDRH2 comprises the sequence as set forth in SEQ ID NO: 352, the CDRH3 comprises the sequence as set forth in SEQ ID NO: 353, the CDRL1 comprises the sequence as set forth in SEQ ID NO: 354, the CDRL2 comprises the sequence as set forth in SEQ ID NO: 355, and the CDRL3 comprises the sequence as set forth in SEQ ID NO: 356, according to Contact;
wherein Z156-02 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 357, the CDRH2 comprises the sequence as set forth in SEQ ID NO: 358, the CDRH3 comprises the sequence as set forth in SEQ ID NO: 359, the CDRL1 comprises the sequence as set forth in SEQ ID NO: 360, the CDRL2 comprises the sequence as set forth in SEQ ID NO: 361, and the CDRL3 comprises the sequence as set forth in SEQ ID NO: 362, according to North;
wherein the CDRH1 comprises the sequence as set forth in SEQ ID NO: 363, the CDRH2 comprises the sequence as set forth in SEQ ID NO: 364, the CDRH3 comprises the sequence as set forth in SEQ ID NO: 365, the CDRL1 comprises the sequence as set forth in SEQ ID NO: 360, the CDRL2 comprises the sequence as set forth in SEQ ID NO: 366, and the CDRL3 comprises the sequence as set forth in SEQ ID NO: 362, according to Kabat;
wherein the CDRH1 comprises the sequence as set forth in SEQ ID NO: 367, the CDRH2 comprises the sequence as set forth in SEQ ID NO: 368, the CDRH3 comprises the sequence as set forth in SEQ ID NO: 359, the CDRL1 comprises the sequence as set forth in SEQ ID NO: 369, the CDRL2 comprises the sequence KD, and the CDRL3 comprises the sequence as set forth in SEQ ID NO: 362 according to IMGT;
wherein the CDRH1 comprises the sequence as set forth in SEQ ID NO: 370, the CDRH2 comprises the sequence as set forth in SEQ ID NO: 371, the CDRH3 comprises the sequence as set forth in SEQ ID NO: 365, the CDRL1 comprises the sequence as set forth in SEQ ID NO: 360, the CDRL2 comprises the sequence as set forth in SEQ ID NO: 366, and the CDRL3 comprises the sequence as set forth in SEQ ID NO: 362, according to Chothia; or
wherein the CDRH1 comprises the sequence as set forth in SEQ ID NO: 372 the CDRH2 comprises the sequence as set forth in SEQ ID NO: 373, the CDRH3 comprises the sequence as set forth in SEQ ID NO: 374 the CDRL1 comprises the sequence as set forth in SEQ ID NO: 74, the CDRL2 comprises the sequence as set forth in SEQ ID NO: 375, and the CDRL3 comprises the sequence as set forth in SEQ ID NO: 376, according to Contact;
wherein Z156-03 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 377, the CDRH2 comprises the sequence as set forth in SEQ ID NO: 378, the CDRH3 comprises the sequence as set forth in SEQ ID NO: 379, the CDRL1 comprises the sequence as set forth in SEQ ID NO: 380, the CDRL2 comprises the sequence as set forth in SEQ ID NO: 361, and the CDRL3 comprises the sequence as set forth in SEQ ID NO: 381, according to North;
wherein the CDRH1 comprises the sequence as set forth in SEQ ID NO: 85, the CDRH2 comprises the sequence as set forth in SEQ ID NO: 382, the CDRH3 comprises the sequence as set forth in SEQ ID NO: 383, the CDRL1 comprises the sequence as set forth in SEQ ID NO: 380, the CDRL2 comprises the sequence as set forth in SEQ ID NO: 366, and the CDRL3 comprises the sequence as set forth in SEQ ID NO: 381, according to Kabat;
wherein the CDRH1 comprises the sequence as set forth in SEQ ID NO: 384, the CDRH2 comprises the sequence as set forth in SEQ ID NO: 385, the CDRH3 comprises the sequence as set forth in SEQ ID NO: 379, the CDRL1 comprises the sequence as set forth in SEQ ID NO: 386, the CDRL2 comprises the sequence KD, and the CDRL3 comprises the sequence as set forth in SEQ ID NO: 381 according to IMGT;
wherein the CDRH1 comprises the sequence as set forth in SEQ ID NO: 387, the CDRH2 comprises the sequence as set forth in SEQ ID NO: 388, the CDRH3 comprises the sequence as set forth in SEQ ID NO: 383, the CDRL1 comprises the sequence as set forth in SEQ ID NO: 380, the CDRL2 comprises the sequence as set forth in SEQ ID NO: 366, and the CDRL3 comprises the sequence as set forth in SEQ ID NO: 381, according to Chothia; or
wherein the CDRH1 comprises the sequence as set forth in SEQ ID NO: 94 the CDRH2 comprises the sequence as set forth in SEQ ID NO: 389, the CDRH3 comprises the sequence as set forth in SEQ ID NO: 390, the CDRL1 comprises the sequence as set forth in SEQ ID NO: 391, the CDRL2 comprises the sequence as set forth in SEQ ID NO: 375, and the CDRL3 comprises the sequence as set forth in SEQ ID NO: 392, according to Contact;
wherein Z249-01 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 393, the CDRH2 comprises the sequence as set forth in SEQ ID NO: 394, the CDRH3 comprises the sequence as set forth in SEQ ID NO: 395, the CDRL1 comprises the sequence as set forth in SEQ ID NO: 396, the CDRL2 comprises the sequence as set forth in SEQ ID NO: 397, and the CDRL3 comprises the sequence as set forth in SEQ ID NO: 398, according to North;
wherein the CDRH1 comprises the sequence as set forth in SEQ ID NO: 399, the CDRH2 comprises the sequence as set forth in SEQ ID NO: 400, the CDRH3 comprises the sequence as set forth in SEQ ID NO: 401, the CDRL1 comprises the sequence as set forth in SEQ ID NO: 396, the CDRL2 comprises the sequence as set forth in SEQ ID NO: 402, and the CDRL3 comprises the sequence as set forth in SEQ ID NO: 398, according to Kabat;
wherein the CDRH1 comprises the sequence as set forth in SEQ ID NO: 403, the CDRH2 comprises the sequence as set forth in SEQ ID NO: 404, the CDRH3 comprises the sequence as set forth in SEQ ID NO: 395, the CDRL1 comprises the sequence as set forth in SEQ ID NO: 405, the CDRL2 comprises the sequence KD, and the CDRL3 comprises the sequence as set forth in SEQ ID NO: 398 according to IMGT;
wherein the CDRH1 comprises the sequence as set forth in SEQ ID NO: 406, the CDRH2 comprises the sequence as set forth in SEQ ID NO: 407, the CDRH3 comprises the sequence as set forth in SEQ ID NO: 401, the CDRL1 comprises the sequence as set forth in SEQ ID NO: 396, the CDRL2 comprises the sequence as set forth in SEQ ID NO: 402, and the CDRL3 comprises the sequence as set forth in SEQ ID NO: 398, according to Chothia; or
wherein the CDRH1 comprises the sequence as set forth in SEQ ID NO: 408, the CDRH2 comprises the sequence as set forth in SEQ ID NO: 409, the CDRH3 comprises the sequence as set forth in SEQ ID NO: 410, the CDRL1 comprises the sequence as set forth in SEQ ID NO: 411, the CDRL2 comprises the sequence as set forth in SEQ ID NO: 412, and the CDRL3 comprises the sequence as set forth in SEQ ID NO: 413, according to Contact;
wherein Z249-02 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 414, the CDRH2 comprises the sequence as set forth in SEQ ID NO: 415, the CDRH3 comprises the sequence as set forth in SEQ ID NO: 416, the CDRL1 comprises the sequence as set forth in SEQ ID NO: 417, the CDRL2 comprises the sequence as set forth in SEQ ID NO: 418, and the CDRL3 comprises the sequence as set forth in SEQ ID NO: 419, according to North;
wherein the CDRH1 comprises the sequence as set forth in SEQ ID NO: 420, the CDRH2 comprises the sequence as set forth in SEQ ID NO: 421, the CDRH3 comprises the sequence as set forth in SEQ ID NO: 422, the CDRL1 comprises the sequence as set forth in SEQ ID NO: 417, the CDRL2 comprises the sequence as set forth in SEQ ID NO: 423, and the CDRL3 comprises the sequence as set forth in SEQ ID NO: 419, according to Kabat;
wherein the CDRH1 comprises the sequence as set forth in SEQ ID NO: 424, the CDRH2 comprises the sequence as set forth in SEQ ID NO: 425, the CDRH3 comprises the sequence as set forth in SEQ ID NO: 416, the CDRL1 comprises the sequence as set forth in SEQ ID NO: 426, the CDRL2 comprises the sequence GA, and the CDRL3 comprises the sequence as set forth in SEQ ID NO: 419 according to IMGT;
wherein the CDRH1 comprises the sequence as set forth in SEQ ID NO: 427, the CDRH2 comprises the sequence as set forth in SEQ ID NO: 428, the CDRH3 comprises the sequence as set forth in SEQ ID NO: 422, the CDRL1 comprises the sequence as set forth in SEQ ID NO: 417, the CDRL2 comprises the sequence as set forth in SEQ ID NO: 423, and the CDRL3 comprises the sequence as set forth in SEQ ID NO: 419, according to Chothia; or
wherein the CDRH1 comprises the sequence as set forth in SEQ ID NO: 429, the CDRH2 comprises the sequence as set forth in SEQ ID NO: 430, the CDRH3 comprises the sequence as set forth in SEQ ID NO: 431, the CDRL1 comprises the sequence as set forth in SEQ ID NO: 432, the CDRL2 comprises the sequence as set forth in SEQ ID NO: 433, and the CDRL3 comprises the sequence as set forth in SEQ ID NO: 434, according to Contact;
wherein Z249-03 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 435, the CDRH2 comprises the sequence as set forth in SEQ ID NO: 436, the CDRH3 comprises the sequence as set forth in SEQ ID NO: 437, the CDRL1 comprises the sequence as set forth in SEQ ID NO: 438, the CDRL2 comprises the sequence as set forth in SEQ ID NO: 439, and the CDRL3 comprises the sequence as set forth in SEQ ID NO: 440, according to North;
wherein the CDRH1 comprises the sequence as set forth in SEQ ID NO: 441, the CDRH2 comprises the sequence as set forth in SEQ ID NO: 442, the CDRH3 comprises the sequence as set forth in SEQ ID NO: 443, the CDRL1 comprises the sequence as set forth in SEQ ID NO: 438, the CDRL2 comprises the sequence as set forth in SEQ ID NO: 444, and the CDRL3 comprises the sequence as set forth in SEQ ID NO: 440, according to Kabat;
wherein the CDRH1 comprises the sequence as set forth in SEQ ID NO: 445, the CDRH2 comprises the sequence as set forth in SEQ ID NO: 446, the CDRH3 comprises the sequence as set forth in SEQ ID NO: 437, the CDRL1 comprises the sequence as set forth in SEQ ID NO: 447, the CDRL2 comprises the sequence DV, and the CDRL3 comprises the sequence as set forth in SEQ ID NO: 440 according to IMGT;
wherein the CDRH1 comprises the sequence as set forth in SEQ ID NO: 448, the CDRH2 comprises the sequence as set forth in SEQ ID NO: 449, the CDRH3 comprises the sequence as set forth in SEQ ID NO: 443, the CDRL1 comprises the sequence as set forth in SEQ ID NO: 438, the CDRL2 comprises the sequence as set forth in SEQ ID NO: 444, and the CDRL3 comprises the sequence as set forth in SEQ ID NO: 440, according to Chothia; or
wherein the CDRH1 comprises the sequence as set forth in SEQ ID NO: 450, the CDRH2 comprises the sequence as set forth in SEQ ID NO: 451, the CDRH3 comprises the sequence as set forth in SEQ ID NO: 452, the CDRL1 comprises the sequence as set forth in SEQ ID NO: 289, the CDRL2 comprises the sequence as set forth in SEQ ID NO: 453, and the CDRL3 comprises the sequence as set forth in SEQ ID NO: 454, according to Contact;
wherein Z249-04 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 455, the CDRH2 comprises the sequence as set forth in SEQ ID NO: 456, the CDRH3 comprises the sequence as set forth in SEQ ID NO: 457, the CDRL1 comprises the sequence as set forth in SEQ ID NO: 458, the CDRL2 comprises the sequence as set forth in SEQ ID NO: 361, and the CDRL3 comprises the sequence as set forth in SEQ ID NO: 459, according to North;
wherein the CDRH1 comprises the sequence as set forth in SEQ ID NO: 460, the CDRH2 comprises the sequence as set forth in SEQ ID NO: 461, the CDRH3 comprises the sequence as set forth in SEQ ID NO: 462, the CDRL1 comprises the sequence as set forth in SEQ ID NO: 458, the CDRL2 comprises the sequence as set forth in SEQ ID NO: 366, and the CDRL3 comprises the sequence as set forth in SEQ ID NO: 459, according to Kabat;
wherein the CDRH1 comprises the sequence as set forth in SEQ ID NO: 463, the CDRH2 comprises the sequence as set forth in SEQ ID NO: 464, the CDRH3 comprises the sequence as set forth in SEQ ID NO: 457, the CDRL1 comprises the sequence as set forth in SEQ ID NO: 465, the CDRL2 comprises the sequence KD, and the CDRL3 comprises the sequence as set forth in SEQ ID NO: 459 according to IMGT;
wherein the CDRH1 comprises the sequence as set forth in SEQ ID NO: 466, the CDRH2 comprises the sequence as set forth in SEQ ID NO: 467, the CDRH3 comprises the sequence as set forth in SEQ ID NO: 462, the CDRL1 comprises the sequence as set forth in SEQ ID NO: 458, the CDRL2 comprises the sequence as set forth in SEQ ID NO: 366, and the CDRL3 comprises the sequence as set forth in SEQ ID NO: 459, according to Chothia; or
wherein the CDRH1 comprises the sequence as set forth in SEQ ID NO: 468, the CDRH2 comprises the sequence as set forth in SEQ ID NO: 469, the CDRH3 comprises the sequence as set forth in SEQ ID NO: 470, the CDRL1 comprises the sequence as set forth in SEQ ID NO: 471, the CDRL2 comprises the sequence as set forth in SEQ ID NO: 472, and the CDRL3 comprises the sequence as set forth in SEQ ID NO: 473, according to Contact;
wherein Z249-05 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 474, the CDRH2 comprises the sequence as set forth in SEQ ID NO: 475, the CDRH3 comprises the sequence as set forth in SEQ ID NO: 476, the CDRL1 comprises the sequence as set forth in SEQ ID NO: 477, the CDRL2 comprises the sequence as set forth in SEQ ID NO: 61, and the CDRL3 comprises the sequence as set forth in SEQ ID NO: 478, according to North;
wherein the CDRH1 comprises the sequence as set forth in SEQ ID NO: 479, the CDRH2 comprises the sequence as set forth in SEQ ID NO: 480, the CDRH3 comprises the sequence as set forth in SEQ ID NO: 481, the CDRL1 comprises the sequence as set forth in SEQ ID NO: 477, the CDRL2 comprises the sequence as set forth in SEQ ID NO: 41, and the CDRL3 comprises the sequence as set forth in SEQ ID NO: 478, according to Kabat;
wherein the CDRH1 comprises the sequence as set forth in SEQ ID NO: 482, the CDRH2 comprises the sequence as set forth in SEQ ID NO: 483, the CDRH3 comprises the sequence as set forth in SEQ ID NO: 476, the CDRL1 comprises the sequence as set forth in SEQ ID NO: 484, the CDRL2 comprises the sequence KD, and the CDRL3 comprises the sequence as set forth in SEQ ID NO: 478 according to IMGT;
wherein the CDRH1 comprises the sequence as set forth in SEQ ID NO: 485, the CDRH2 comprises the sequence as set forth in SEQ ID NO: 486, the CDRH3 comprises the sequence as set forth in SEQ ID NO: 481, the CDRL1 comprises the sequence as set forth in SEQ ID NO: 477, the CDRL2 comprises the sequence as set forth in SEQ ID NO: 66, and the CDRL3 comprises the sequence as set forth in SEQ ID NO: 478, according to Chothia; or
wherein the CDRH1 comprises the sequence as set forth in SEQ ID NO: 487, the CDRH2 comprises the sequence as set forth in SEQ ID NO: 488, the CDRH3 comprises the sequence as set forth in SEQ ID NO: 489, the CDRL1 comprises the sequence as set forth in SEQ ID NO: 490, the CDRL2 comprises the sequence as set forth in SEQ ID NO: 491, and the CDRL3 comprises the sequence as set forth in SEQ ID NO: 491, according to Contact;
wherein Z249-06 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 414, the CDRH2 comprises the sequence as set forth in SEQ ID NO: 415, the CDRH3 comprises the sequence as set forth in SEQ ID NO: 416, the CDRL1 comprises the sequence as set forth in SEQ ID NO: 417, the CDRL2 comprises the sequence as set forth in SEQ ID NO: 418, and the CDRL3 comprises the sequence as set forth in SEQ ID NO: 419, according to North;
wherein the CDRH1 comprises the sequence as set forth in SEQ ID NO: 420, the CDRH2 comprises the sequence as set forth in SEQ ID NO: 421, the CDRH3 comprises the sequence as set forth in SEQ ID NO: 422, the CDRL1 comprises the sequence as set forth in SEQ ID NO: 417, the CDRL2 comprises the sequence as set forth in SEQ ID NO: 423, and the CDRL3 comprises the sequence as set forth in SEQ ID NO: 419, according to Kabat;
wherein the CDRH1 comprises the sequence as set forth in SEQ ID NO: 424, the CDRH2 comprises the sequence as set forth in SEQ ID NO: 425, the CDRH3 comprises the sequence as set forth in SEQ ID NO: 416, the CDRL1 comprises the sequence as set forth in SEQ ID NO: 426, the CDRL2 comprises the sequence GA, and the CDRL3 comprises the sequence as set forth in SEQ ID NO: 419 according to IMGT;
wherein the CDRH1 comprises the sequence as set forth in SEQ ID NO: 427, the CDRH2 comprises the sequence as set forth in SEQ ID NO: 428, the CDRH3 comprises the sequence as set forth in SEQ ID NO: 422, the CDRL1 comprises the sequence as set forth in SEQ ID NO: 417, the CDRL2 comprises the sequence as set forth in SEQ ID NO: 423, and the CDRL3 comprises the sequence as set forth in SEQ ID NO: 419, according to Chothia; or
wherein the CDRH1 comprises the sequence as set forth in SEQ ID NO: 429, the CDRH2 comprises the sequence as set forth in SEQ ID NO: 430, the CDRH3 comprises the sequence as set forth in SEQ ID NO: 431, the CDRL1 comprises the sequence as set forth in SEQ ID NO: 432, the CDRL2 comprises the sequence as set forth in SEQ ID NO: 433, and the CDRL3 comprises the sequence as set forth in SEQ ID NO: 434, according to Contact;
wherein Z308-03 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 493, the CDRH2 comprises the sequence as set forth in SEQ ID NO: 494, the CDRH3 comprises the sequence as set forth in SEQ ID NO: 495, the CDRL1 comprises the sequence as set forth in SEQ ID NO: 496, the CDRL2 comprises the sequence as set forth in SEQ ID NO: 497, and the CDRL3 comprises the sequence as set forth in SEQ ID NO: 498, according to North;
wherein the CDRH1 comprises the sequence as set forth in SEQ ID NO: 499, the CDRH2 comprises the sequence as set forth in SEQ ID NO: 500, the CDRH3 comprises the sequence as set forth in SEQ ID NO: 501, the CDRL1 comprises the sequence as set forth in SEQ ID NO: 496, the CDRL2 comprises the sequence as set forth in SEQ ID NO: 502, and the CDRL3 comprises the sequence as set forth in SEQ ID NO: 498, according to Kabat;
wherein the CDRH1 comprises the sequence as set forth in SEQ ID NO: 503, the CDRH2 comprises the sequence as set forth in SEQ ID NO: 504, the CDRH3 comprises the sequence as set forth in SEQ ID NO: 495, the CDRL1 comprises the sequence as set forth in SEQ ID NO: 505, the CDRL2 comprises the sequence SA, and the CDRL3 comprises the sequence as set forth in SEQ ID NO: 498 according to IMGT;
wherein the CDRH1 comprises the sequence as set forth in SEQ ID NO: 506, the CDRH2 comprises the sequence as set forth in SEQ ID NO: 507, the CDRH3 comprises the sequence as set forth in SEQ ID NO: 501, the CDRL1 comprises the sequence as set forth in SEQ ID NO: 496, the CDRL2 comprises the sequence as set forth in SEQ ID NO: 502, and the CDRL3 comprises the sequence as set forth in SEQ ID NO: 498, according to Chothia; or
wherein the CDRH1 comprises the sequence as set forth in SEQ ID NO: 508, the CDRH2 comprises the sequence as set forth in SEQ ID NO: 509, the CDRH3 comprises the sequence as set forth in SEQ ID NO: 510, the CDRL1 comprises the sequence as set forth in SEQ ID NO: 511, the CDRL2 comprises the sequence as set forth in SEQ ID NO: 512, and the CDRL3 comprises the sequence as set forth in SEQ ID NO: 513, according to Contact;
wherein Z308-04 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 514, the CDRH2 comprises the sequence as set forth in SEQ ID NO: 515, the CDRH3 comprises the sequence as set forth in SEQ ID NO: 516, the CDRL1 comprises the sequence as set forth in SEQ ID NO: 517, the CDRL2 comprises the sequence as set forth in SEQ ID NO: 518, and the CDRL3 comprises the sequence as set forth in SEQ ID NO: 519, according to North;
wherein the CDRH1 comprises the sequence as set forth in SEQ ID NO: 520, the CDRH2 comprises the sequence as set forth in SEQ ID NO: 521, the CDRH3 comprises the sequence as set forth in SEQ ID NO: 522, the CDRL1 comprises the sequence as set forth in SEQ ID NO: 517, the CDRL2 comprises the sequence as set forth in SEQ ID NO: 523, and the CDRL3 comprises the sequence as set forth in SEQ ID NO: 519, according to Kabat;
wherein the CDRH1 comprises the sequence as set forth in SEQ ID NO: 524, the CDRH2 comprises the sequence as set forth in SEQ ID NO: 525, the CDRH3 comprises the sequence as set forth in SEQ ID NO: 516, the CDRL1 comprises the sequence as set forth in SEQ ID NO: 526, the CDRL2 comprises the sequence TA, and the CDRL3 comprises the sequence as set forth in SEQ ID NO: 519 according to IMGT;
wherein the CDRH1 comprises the sequence as set forth in SEQ ID NO: 527, the CDRH2 comprises the sequence as set forth in SEQ ID NO: 528, the CDRH3 comprises the sequence as set forth in SEQ ID NO: 522, the CDRL1 comprises the sequence as set forth in SEQ ID NO: 517, the CDRL2 comprises the sequence as set forth in SEQ ID NO: 523, and the CDRL3 comprises the sequence as set forth in SEQ ID NO: 519, according to Chothia; or
wherein the CDRH1 comprises the sequence as set forth in SEQ ID NO: 529, the CDRH2 comprises the sequence as set forth in SEQ ID NO: 530, the CDRH3 comprises the sequence as set forth in SEQ ID NO: 531, the CDRL1 comprises the sequence as set forth in SEQ ID NO: 532, the CDRL2 comprises the sequence as set forth in SEQ ID NO: 533, and the CDRL3 comprises the sequence as set forth in SEQ ID NO: 534, according to Contact;
wherein Z308-05 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 535, the CDRH2 comprises the sequence as set forth in SEQ ID NO: 272, the CDRH3 comprises the sequence as set forth in SEQ ID NO: 536, the CDRL1 comprises the sequence as set forth in SEQ ID NO: 537, the CDRL2 comprises the sequence as set forth in SEQ ID NO: 497, and the CDRL3 comprises the sequence as set forth in SEQ ID NO: 538, according to North;
wherein the CDRH1 comprises the sequence as set forth in SEQ ID NO: 539, the CDRH2 comprises the sequence as set forth in SEQ ID NO: 540, the CDRH3 comprises the sequence as set forth in SEQ ID NO: 541, the CDRL1 comprises the sequence as set forth in SEQ ID NO: 537, the CDRL2 comprises the sequence as set forth in SEQ ID NO: 502, and the CDRL3 comprises the sequence as set forth in SEQ ID NO: 538, according to Kabat;
wherein the CDRH1 comprises the sequence as set forth in SEQ ID NO: 542, the CDRH2 comprises the sequence as set forth in SEQ ID NO: 282, the CDRH3 comprises the sequence as set forth in SEQ ID NO: 536, the CDRL1 comprises the sequence as set forth in SEQ ID NO: 542, the CDRL2 comprises the sequence SA, and the CDRL3 comprises the sequence as set forth in SEQ ID NO: 538 according to IMGT;
wherein the CDRH1 comprises the sequence as set forth in SEQ ID NO: 543, the CDRH2 comprises the sequence as set forth in SEQ ID NO: 285, the CDRH3 comprises the sequence as set forth in SEQ ID NO: 541, the CDRL1 comprises the sequence as set forth in SEQ ID NO: 537, the CDRL2 comprises the sequence as set forth in SEQ ID NO: 502, and the CDRL3 comprises the sequence as set forth in SEQ ID NO: 538, according to Chothia; or
wherein the CDRH1 comprises the sequence as set forth in SEQ ID NO: 544, the CDRH2 comprises the sequence as set forth in SEQ ID NO: 287, the CDRH3 comprises the sequence as set forth in SEQ ID NO: 545, the CDRL1 comprises the sequence as set forth in SEQ ID NO: 546, the CDRL2 comprises the sequence as set forth in SEQ ID NO: 512, and the CDRL3 comprises the sequence as set forth in SEQ ID NO: 547, according to Contact;
wherein Z504-03 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 548, the CDRH2 comprises the sequence as set forth in SEQ ID NO: 549, the CDRH3 comprises the sequence as set forth in SEQ ID NO: 550, the CDRL1 comprises the sequence as set forth in SEQ ID NO: 551, the CDRL2 comprises the sequence as set forth in SEQ ID NO: 552, and the CDRL3 comprises the sequence as set forth in SEQ ID NO: 1308, according to North;
wherein the CDRH1 comprises the sequence as set forth in SEQ ID NO: 553, the CDRH2 comprises the sequence as set forth in SEQ ID NO: 554, the CDRH3 comprises the sequence as set forth in SEQ ID NO: 555, the CDRL1 comprises the sequence as set forth in SEQ ID NO: 551, the CDRL2 comprises the sequence as set forth in SEQ ID NO: 556, and the CDRL3 comprises the sequence as set forth in SEQ ID NO: 1308, according to Kabat;
wherein the CDRH1 comprises the sequence as set forth in SEQ ID NO: 557, the CDRH2 comprises the sequence as set forth in SEQ ID NO: 558, the CDRH3 comprises the sequence as set forth in SEQ ID NO: 550, the CDRL1 comprises the sequence as set forth in SEQ ID NO: 559, the CDRL2 comprises the sequence GA, and the CDRL3 comprises the sequence as set forth in SEQ ID NO: 1308 according to IMGT;
wherein the CDRH1 comprises the sequence as set forth in SEQ ID NO: 560 the CDRH2 comprises the sequence as set forth in SEQ ID NO: 561, the CDRH3 comprises the sequence as set forth in SEQ ID NO: 555, the CDRL1 comprises the sequence as set forth in SEQ ID NO: 551, the CDRL2 comprises the sequence as set forth in SEQ ID NO: 556, and the CDRL3 comprises the sequence as set forth in SEQ ID NO: 1308, according to Chothia; or
wherein the CDRH1 comprises the sequence as set forth in SEQ ID NO: 562, the CDRH2 comprises the sequence as set forth in SEQ ID NO: 563, the CDRH3 comprises the sequence as set forth in SEQ ID NO: 564, the CDRL1 comprises the sequence as set forth in SEQ ID NO: 565, the CDRL2 comprises the sequence as set forth in SEQ ID NO: 566, and the CDRL3 comprises the sequence as set forth in SEQ ID NO: 567, according to Contact;
wherein Z504-04 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 568, the CDRH2 comprises the sequence as set forth in SEQ ID NO: 569, the CDRH3 comprises the sequence as set forth in SEQ ID NO: 570, the CDRL1 comprises the sequence as set forth in SEQ ID NO: 571, the CDRL2 comprises the sequence as set forth in SEQ ID NO: 572, and the CDRL3 comprises the sequence as set forth in SEQ ID NO: 573, according to North;
wherein the CDRH1 comprises the sequence as set forth in SEQ ID NO: 342, the CDRH2 comprises the sequence as set forth in SEQ ID NO: 574, the CDRH3 comprises the sequence as set forth in SEQ ID NO: 570, the CDRL1 comprises the sequence as set forth in SEQ ID NO: 571, the CDRL2 comprises the sequence as set forth in SEQ ID NO: 575, and the CDRL3 comprises the sequence as set forth in SEQ ID NO: 573, according to Kabat;
wherein the CDRH1 comprises the sequence as set forth in SEQ ID NO: 576, the CDRH2 comprises the sequence as set forth in SEQ ID NO: 577, the CDRH3 comprises the sequence as set forth in SEQ ID NO: 570, the CDRL1 comprises the sequence as set forth in SEQ ID NO: 578, the CDRL2 comprises the sequence GA, and the CDRL3 comprises the sequence as set forth in SEQ ID NO: 573 according to IMGT;
wherein the CDRH1 comprises the sequence as set forth in SEQ ID NO: 579, the CDRH2 comprises the sequence as set forth in SEQ ID NO: 580, the CDRH3 comprises the sequence as set forth in SEQ ID NO: 570, the CDRL1 comprises the sequence as set forth in SEQ ID NO: 571, the CDRL2 comprises the sequence as set forth in SEQ ID NO: 575, and the CDRL3 comprises the sequence as set forth in SEQ ID NO: 573, according to Chothia; or
wherein the CDRH1 comprises the sequence as set forth in SEQ ID NO: 351, the CDRH2 comprises the sequence as set forth in SEQ ID NO: 581, the CDRH3 comprises the sequence as set forth in SEQ ID NO: 582, the CDRL1 comprises the sequence as set forth in SEQ ID NO: 583, the CDRL2 comprises the sequence as set forth in SEQ ID NO: 584, and the CDRL3 comprises the sequence as set forth in SEQ ID NO: 585, according to Contact;
wherein Z504-06 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 586, the CDRH2 comprises the sequence as set forth in SEQ ID NO: 569, the CDRH3 comprises the sequence as set forth in SEQ ID NO: 587, the CDRL1 comprises the sequence as set forth in SEQ ID NO: 571, the CDRL2 comprises the sequence as set forth in SEQ ID NO: 572, and the CDRL3 comprises the sequence as set forth in SEQ ID NO: 588, according to North;
wherein the CDRH1 comprises the sequence as set forth in SEQ ID NO: 589, the CDRH2 comprises the sequence as set forth in SEQ ID NO: 574, the CDRH3 comprises the sequence as set forth in SEQ ID NO: 590, the CDRL1 comprises the sequence as set forth in SEQ ID NO: 571, the CDRL2 comprises the sequence as set forth in SEQ ID NO: 575, and the CDRL3 comprises the sequence as set forth in SEQ ID NO: 588, according to Kabat;
wherein the CDRH1 comprises the sequence as set forth in SEQ ID NO: 591, the CDRH2 comprises the sequence as set forth in SEQ ID NO: 577, the CDRH3 comprises the sequence as set forth in SEQ ID NO: 587, the CDRL1 comprises the sequence as set forth in SEQ ID NO: 578, the CDRL2 comprises the sequence GA, and the CDRL3 comprises the sequence as set forth in SEQ ID NO: 588 according to IMGT;
wherein the CDRH1 comprises the sequence as set forth in SEQ ID NO: 592, the CDRH2 comprises the sequence as set forth in SEQ ID NO: 580, the CDRH3 comprises the sequence as set forth in SEQ ID NO: 590, the CDRL1 comprises the sequence as set forth in SEQ ID NO: 571, the CDRL2 comprises the sequence as set forth in SEQ ID NO: 575, and the CDRL3 comprises the sequence as set forth in SEQ ID NO: 588, according to Chothia; or
wherein the CDRH1 comprises the sequence as set forth in SEQ ID NO: 593, the CDRH2 comprises the sequence as set forth in SEQ ID NO: 581, the CDRH3 comprises the sequence as set forth in SEQ ID NO: 594, the CDRL1 comprises the sequence as set forth in SEQ ID NO: 583, the CDRL2 comprises the sequence as set forth in SEQ ID NO: 584, and the CDRL3 comprises the sequence as set forth in SEQ ID NO: 595, according to Contact;
wherein Z504-07 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 596, the CDRH2 comprises the sequence as set forth in SEQ ID NO: 597, the CDRH3 comprises the sequence as set forth in SEQ ID NO: 598, the CDRL1 comprises the sequence as set forth in SEQ ID NO: 599, the CDRL2 comprises the sequence as set forth in SEQ ID NO: 600, and the CDRL3 comprises the sequence as set forth in SEQ ID NO: 601, according to North;
wherein the CDRH1 comprises the sequence as set forth in SEQ ID NO: 602, the CDRH2 comprises the sequence as set forth in SEQ ID NO: 603, the CDRH3 comprises the sequence as set forth in SEQ ID NO: 604, the CDRL1 comprises the sequence as set forth in SEQ ID NO: 599, the CDRL2 comprises the sequence as set forth in SEQ ID NO: 605, and the CDRL3 comprises the sequence as set forth in SEQ ID NO: 601, according to Kabat;
wherein the CDRH1 comprises the sequence as set forth in SEQ ID NO: 606, the CDRH2 comprises the sequence as set forth in SEQ ID NO: 607, the CDRH3 comprises the sequence as set forth in SEQ ID NO: 604, the CDRL1 comprises the sequence as set forth in SEQ ID NO: 608, the CDRL2 comprises the sequence IT, and the CDRL3 comprises the sequence as set forth in SEQ ID NO: 601 according to IMGT;
wherein the CDRH1 comprises the sequence as set forth in SEQ ID NO: 609, the CDRH2 comprises the sequence as set forth in SEQ ID NO: 610, the CDRH3 comprises the sequence as set forth in SEQ ID NO: 604, the CDRL1 comprises the sequence as set forth in SEQ ID NO: 599, the CDRL2 comprises the sequence as set forth in SEQ ID NO: 605, and the CDRL3 comprises the sequence as set forth in SEQ ID NO: 601, according to Chothia; or
wherein the CDRH1 comprises the sequence as set forth in SEQ ID NO: 611, the CDRH2 comprises the sequence as set forth in SEQ ID NO: 612, the CDRH3 comprises the sequence as set forth in SEQ ID NO: 613, the CDRL1 comprises the sequence as set forth in SEQ ID NO: 614, the CDRL2 comprises the sequence as set forth in SEQ ID NO: 615, and the CDRL3 comprises the sequence as set forth in SEQ ID NO: 616, according to Contact;
wherein Z513-01 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 617, the CDRH2 comprises the sequence as set forth in SEQ ID NO: 618, the CDRH3 comprises the sequence as set forth in SEQ ID NO: 619, the CDRL1 comprises the sequence as set forth in SEQ ID NO: 620, the CDRL2 comprises the sequence as set forth in SEQ ID NO: 621, and the CDRL3 comprises the sequence as set forth in SEQ ID NO: 622, according to North;
wherein the CDRH1 comprises the sequence as set forth in SEQ ID NO: 623, the CDRH2 comprises the sequence as set forth in SEQ ID NO: 624, the CDRH3 comprises the sequence as set forth in SEQ ID NO: 625, the CDRL1 comprises the sequence as set forth in SEQ ID NO: 620, the CDRL2 comprises the sequence as set forth in SEQ ID NO: 626, and the CDRL3 comprises the sequence as set forth in SEQ ID NO: 622, according to Kabat;
wherein the CDRH1 comprises the sequence as set forth in SEQ ID NO: 627, the CDRH2 comprises the sequence as set forth in SEQ ID NO: 628, the CDRH3 comprises the sequence as set forth in SEQ ID NO: 625, the CDRL1 comprises the sequence as set forth in SEQ ID NO: 629, the CDRL2 comprises the sequence VA, and the CDRL3 comprises the sequence as set forth in SEQ ID NO: 622 according to IMGT;
wherein the CDRH1 comprises the sequence as set forth in SEQ ID NO: 630, the CDRH2 comprises the sequence as set forth in SEQ ID NO: 631, the CDRH3 comprises the sequence as set forth in SEQ ID NO: 625, the CDRL1 comprises the sequence as set forth in SEQ ID NO: 620, the CDRL2 comprises the sequence as set forth in SEQ ID NO: 626, and the CDRL3 comprises the sequence as set forth in SEQ ID NO: 622, according to Chothia; or
wherein the CDRH1 comprises the sequence as set forth in SEQ ID NO: 632, the CDRH2 comprises the sequence as set forth in SEQ ID NO: 633, the CDRH3 comprises the sequence as set forth in SEQ ID NO: 634, the CDRL1 comprises the sequence as set forth in SEQ ID NO: 635, the CDRL2 comprises the sequence as set forth in SEQ ID NO: 636, and the CDRL3 comprises the sequence as set forth in SEQ ID NO: 637, according to Contact;
wherein Z513-02 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 638, the CDRH2 comprises the sequence as set forth in SEQ ID NO: 639, the CDRH3 comprises the sequence as set forth in SEQ ID NO: 640, the CDRL1 comprises the sequence as set forth in SEQ ID NO: 641, the CDRL2 comprises the sequence as set forth in SEQ ID NO: 642, and the CDRL3 comprises the sequence as set forth in SEQ ID NO: 643, according to North;
wherein the CDRH1 comprises the sequence as set forth in SEQ ID NO: 644, the CDRH2 comprises the sequence as set forth in SEQ ID NO: 645, the CDRH3 comprises the sequence as set forth in SEQ ID NO: 646, the CDRL1 comprises the sequence as set forth in SEQ ID NO: 641, the CDRL2 comprises the sequence as set forth in SEQ ID NO: 647, and the CDRL3 comprises the sequence as set forth in SEQ ID NO: 643, according to Kabat;
wherein the CDRH1 comprises the sequence as set forth in SEQ ID NO: 648, the CDRH2 comprises the sequence as set forth in SEQ ID NO: 649, the CDRH3 comprises the sequence as set forth in SEQ ID NO: 640, the CDRL1 comprises the sequence as set forth in SEQ ID NO: 650, the CDRL2 comprises the sequence AA, and the CDRL3 comprises the sequence as set forth in SEQ ID NO: 643 according to IMGT;
wherein the CDRH1 comprises the sequence as set forth in SEQ ID NO: 651, the CDRH2 comprises the sequence as set forth in SEQ ID NO: 652, the CDRH3 comprises the sequence as set forth in SEQ ID NO: 646, the CDRL1 comprises the sequence as set forth in SEQ ID NO: 641, the CDRL2 comprises the sequence as set forth in SEQ ID NO: 647, and the CDRL3 comprises the sequence as set forth in SEQ ID NO: 643, according to Chothia; or
wherein the CDRH1 comprises the sequence as set forth in SEQ ID NO: 653, the CDRH2 comprises the sequence as set forth in SEQ ID NO: 654, the CDRH3 comprises the sequence as set forth in SEQ ID NO: 655, the CDRL1 comprises the sequence as set forth in SEQ ID NO: 656, the CDRL2 comprises the sequence as set forth in SEQ ID NO: 657, and the CDRL3 comprises the sequence as set forth in SEQ ID NO: 658, according to Contact;
wherein Z538-03 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 659, the CDRH2 comprises the sequence as set forth in SEQ ID NO: 660, the CDRH3 comprises the sequence as set forth in SEQ ID NO: 661, the CDRL1 comprises the sequence as set forth in SEQ ID NO: 641, the CDRL2 comprises the sequence as set forth in SEQ ID NO: 662, and the CDRL3 comprises the sequence as set forth in SEQ ID NO: 663, according to North;
wherein the CDRH1 comprises the sequence as set forth in SEQ ID NO: 665, the CDRH2 comprises the sequence as set forth in SEQ ID NO: 666, the CDRH3 comprises the sequence as set forth in SEQ ID NO: 667, the CDRL1 comprises the sequence as set forth in SEQ ID NO: 662, the CDRL2 comprises the sequence as set forth in SEQ ID NO: 668, and the CDRL3 comprises the sequence as set forth in SEQ ID NO: 664, according to Kabat;
wherein the CDRH1 comprises the sequence as set forth in SEQ ID NO: 669, the CDRH2 comprises the sequence as set forth in SEQ ID NO: 670, the CDRH3 comprises the sequence as set forth in SEQ ID NO: 661, the CDRL1 comprises the sequence as set forth in SEQ ID NO: 671, the CDRL2 comprises the sequence as set forth in SEQ ID NO: 672, and the CDRL3 comprises the sequence as set forth in SEQ ID NO: 664 according to IMGT;
wherein the CDRH1 comprises the sequence as set forth in SEQ ID NO: 673, the CDRH2 comprises the sequence as set forth in SEQ ID NO: 674, the CDRH3 comprises the sequence as set forth in SEQ ID NO: 667, the CDRL1 comprises the sequence as set forth in SEQ ID NO: 662, the CDRL2 comprises the sequence as set forth in SEQ ID NO: 668, and the CDRL3 comprises the sequence as set forth in SEQ ID NO: 664, according to Chothia; or
wherein the CDRH1 comprises the sequence as set forth in SEQ ID NO: 675, the CDRH2 comprises the sequence as set forth in SEQ ID NO: 676, the CDRH3 comprises the sequence as set forth in SEQ ID NO: 677, the CDRL1 comprises the sequence as set forth in SEQ ID NO: 678, the CDRL2 comprises the sequence as set forth in SEQ ID NO: 679, and the CDRL3 comprises the sequence as set forth in SEQ ID NO: 680, according to Contact;
wherein Z573-03 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 681, the CDRH2 comprises the sequence as set forth in SEQ ID NO: 682, the CDRH3 comprises the sequence as set forth in SEQ ID NO: 683, the CDRL1 comprises the sequence as set forth in SEQ ID NO: 684, the CDRL2 comprises the sequence as set forth in SEQ ID NO: 361, and the CDRL3 comprises the sequence as set forth in SEQ ID NO: 685, according to North;
wherein the CDRH1 comprises the sequence as set forth in SEQ ID NO: 686, the CDRH2 comprises the sequence as set forth in SEQ ID NO: 687, the CDRH3 comprises the sequence as set forth in SEQ ID NO: 688, the CDRL1 comprises the sequence as set forth in SEQ ID NO: 684, the CDRL2 comprises the sequence as set forth in SEQ ID NO: 366, and the CDRL3 comprises the sequence as set forth in SEQ ID NO: 685, according to Kabat;
wherein the CDRH1 comprises the sequence as set forth in SEQ ID NO: 689, the CDRH2 comprises the sequence as set forth in SEQ ID NO: 690, the CDRH3 comprises the sequence as set forth in SEQ ID NO: 683, the CDRL1 comprises the sequence as set forth in SEQ ID NO: 691, the CDRL2 comprises the sequence KD, and the CDRL3 comprises the sequence as set forth in SEQ ID NO: 685 according to IMGT;
wherein the CDRH1 comprises the sequence as set forth in SEQ ID NO: 692, the CDRH2 comprises the sequence as set forth in SEQ ID NO: 693, the CDRH3 comprises the sequence as set forth in SEQ ID NO: 688, the CDRL1 comprises the sequence as set forth in SEQ ID NO: 684, the CDRL2 comprises the sequence as set forth in SEQ ID NO: 366, and the CDRL3 comprises the sequence as set forth in SEQ ID NO: 685, according to Chothia; or
wherein the CDRH1 comprises the sequence as set forth in SEQ ID NO: 694, the CDRH2 comprises the sequence as set forth in SEQ ID NO: 695, the CDRH3 comprises the sequence as set forth in SEQ ID NO: 696, the CDRL1 comprises the sequence as set forth in SEQ ID NO: 697, the CDRL2 comprises the sequence as set forth in SEQ ID NO: 375, and the CDRL3 comprises the sequence as set forth in SEQ ID NO: 698, according to Contact;
wherein Z582-03 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 699, the CDRH2 comprises the sequence as set forth in SEQ ID NO: 700, the CDRH3 comprises the sequence as set forth in SEQ ID NO: 701, the CDRL1 comprises the sequence as set forth in SEQ ID NO: 702, the CDRL2 comprises the sequence as set forth in SEQ ID NO: 703, and the CDRL3 comprises the sequence as set forth in SEQ ID NO: 704, according to North;
wherein the CDRH1 comprises the sequence as set forth in SEQ ID NO: 705, the CDRH2 comprises the sequence as set forth in SEQ ID NO: 706, the CDRH3 comprises the sequence as set forth in SEQ ID NO: 707, the CDRL1 comprises the sequence as set forth in SEQ ID NO: 702, the CDRL2 comprises the sequence as set forth in SEQ ID NO: 708, and the CDRL3 comprises the sequence as set forth in SEQ ID NO: 704, according to Kabat;
wherein the CDRH1 comprises the sequence as set forth in SEQ ID NO: 709, the CDRH2 comprises the sequence as set forth in SEQ ID NO: 710, the CDRH3 comprises the sequence as set forth in SEQ ID NO: 701, the CDRL1 comprises the sequence as set forth in SEQ ID NO: 711, the CDRL2 comprises the sequence GA, and the CDRL3 comprises the sequence as set forth in SEQ ID NO: 704 according to IMGT;
wherein the CDRH1 comprises the sequence as set forth in SEQ ID NO: 712, the CDRH2 comprises the sequence as set forth in SEQ ID NO: 713, the CDRH3 comprises the sequence as set forth in SEQ ID NO: 707, the CDRL1 comprises the sequence as set forth in SEQ ID NO: 702, the CDRL2 comprises the sequence as set forth in SEQ ID NO: 708, and the CDRL3 comprises the sequence as set forth in SEQ ID NO: 704, according to Chothia; or
wherein the CDRH1 comprises the sequence as set forth in SEQ ID NO: 714, the CDRH2 comprises the sequence as set forth in SEQ ID NO: 715, the CDRH3 comprises the sequence as set forth in SEQ ID NO: 716, the CDRL1 comprises the sequence as set forth in SEQ ID NO: 565, the CDRL2 comprises the sequence as set forth in SEQ ID NO: 717, and the CDRL3 comprises the sequence as set forth in SEQ ID NO: 718, according to Contact;
wherein Z582-14 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 719, the CDRH2 comprises the sequence as set forth in SEQ ID NO: 720, the CDRH3 comprises the sequence as set forth in SEQ ID NO: 721, the CDRL1 comprises the sequence as set forth in SEQ ID NO: 722, the CDRL2 comprises the sequence as set forth in SEQ ID NO: 723, and the CDRL3 comprises the sequence as set forth in SEQ ID NO: 724, according to North;
wherein the CDRH1 comprises the sequence as set forth in SEQ ID NO: 725, the CDRH2 comprises the sequence as set forth in SEQ ID NO: 726, the CDRH3 comprises the sequence as set forth in SEQ ID NO: 727, the CDRL1 comprises the sequence as set forth in SEQ ID NO: 722, the CDRL2 comprises the sequence as set forth in SEQ ID NO: 728, and the CDRL3 comprises the sequence as set forth in SEQ ID NO: 724, according to Kabat;
wherein the CDRH1 comprises the sequence as set forth in SEQ ID NO: 729, the CDRH2 comprises the sequence as set forth in SEQ ID NO: 730, the CDRH3 comprises the sequence as set forth in SEQ ID NO: 721, the CDRL1 comprises the sequence as set forth in SEQ ID NO: 731, the CDRL2 comprises the sequence ED, and the CDRL3 comprises the sequence as set forth in SEQ ID NO: 724 according to IMGT;
wherein the CDRH1 comprises the sequence as set forth in SEQ ID NO: 732, the CDRH2 comprises the sequence as set forth in SEQ ID NO: 733, the CDRH3 comprises the sequence as set forth in SEQ ID NO: 727, the CDRL1 comprises the sequence as set forth in SEQ ID NO: 722, the CDRL2 comprises the sequence as set forth in SEQ ID NO: 728, and the CDRL3 comprises the sequence as set forth in SEQ ID NO: 724, according to Chothia; or
wherein the CDRH1 comprises the sequence as set forth in SEQ ID NO: 734, the CDRH2 comprises the sequence as set forth in SEQ ID NO: 735, the CDRH3 comprises the sequence as set forth in SEQ ID NO: 736, the CDRL1 comprises the sequence as set forth in SEQ ID NO: 737, the CDRL2 comprises the sequence as set forth in SEQ ID NO: 738, and the CDRL3 comprises the sequence as set forth in SEQ ID NO: 739, according to Contact;
wherein Z582-15 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 719, the CDRH2 comprises the sequence as set forth in SEQ ID NO: 720, the CDRH3 comprises the sequence as set forth in SEQ ID NO: 721, the CDRL1 comprises the sequence as set forth in SEQ ID NO: 740, the CDRL2 comprises the sequence as set forth in SEQ ID NO: 741, and the CDRL3 comprises the sequence as set forth in SEQ ID NO: 742, according to North;
wherein the CDRH1 comprises the sequence as set forth in SEQ ID NO: 725, the CDRH2 comprises the sequence as set forth in SEQ ID NO: 726, the CDRH3 comprises the sequence as set forth in SEQ ID NO: 727, the CDRL1 comprises the sequence as set forth in SEQ ID NO: 740, the CDRL2 comprises the sequence as set forth in SEQ ID NO: 743, and the CDRL3 comprises the sequence as set forth in SEQ ID NO: 742, according to Kabat;
wherein the CDRH1 comprises the sequence as set forth in SEQ ID NO: 729, the CDRH2 comprises the sequence as set forth in SEQ ID NO: 730, the CDRH3 comprises the sequence as set forth in SEQ ID NO: 721, the CDRL1 comprises the sequence as set forth in SEQ ID NO: 744, the CDRL2 comprises the sequence YD, and the CDRL3 comprises the sequence as set forth in SEQ ID NO: 742 according to IMGT;
wherein the CDRH1 comprises the sequence as set forth in SEQ ID NO: 732, the CDRH2 comprises the sequence as set forth in SEQ ID NO: 733, the CDRH3 comprises the sequence as set forth in SEQ ID NO: 727, the CDRL1 comprises the sequence as set forth in SEQ ID NO: 740, the CDRL2 comprises the sequence as set forth in SEQ ID NO: 743, and the CDRL3 comprises the sequence as set forth in SEQ ID NO: 742, according to Chothia; or
wherein the CDRH1 comprises the sequence as set forth in SEQ ID NO: 734, the CDRH2 comprises the sequence as set forth in SEQ ID NO: 735, the CDRH3 comprises the sequence as set forth in SEQ ID NO: 736, the CDRL1 comprises the sequence as set forth in SEQ ID NO: 745, the CDRL2 comprises the sequence as set forth in SEQ ID NO: 746, and the CDRL3 comprises the sequence as set forth in SEQ ID NO: 747, according to Contact;
wherein Z082-U1 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 748, the CDRH2 comprises the sequence as set forth in SEQ ID NO: 749, the CDRH3 comprises the sequence as set forth in SEQ ID NO: 750, the CDRL1 comprises the sequence as set forth in SEQ ID NO: 751, the CDRL2 comprises the sequence as set forth in SEQ ID NO: 752, and the CDRL3 comprises the sequence as set forth in SEQ ID NO: 753, according to North;
wherein the CDRH1 comprises the sequence as set forth in SEQ ID NO: 754, the CDRH2 comprises the sequence as set forth in SEQ ID NO: 755, the CDRH3 comprises the sequence as set forth in SEQ ID NO: 756, the CDRL1 comprises the sequence as set forth in SEQ ID NO: 751, the CDRL2 comprises the sequence as set forth in SEQ ID NO: 757, and the CDRL3 comprises the sequence as set forth in SEQ ID NO: 753, according to Kabat;
wherein the CDRH1 comprises the sequence as set forth in SEQ ID NO: 758, the CDRH2 comprises the sequence as set forth in SEQ ID NO: 759, the CDRH3 comprises the sequence as set forth in SEQ ID NO: 750, the CDRL1 comprises the sequence as set forth in SEQ ID NO: 760, the CDRL2 comprises the sequence DV, and the CDRL3 comprises the sequence as set forth in SEQ ID NO: 753 according to IMGT;
wherein the CDRH1 comprises the sequence as set forth in SEQ ID NO: 761, the CDRH2 comprises the sequence as set forth in SEQ ID NO: 762, the CDRH3 comprises the sequence as set forth in SEQ ID NO: 756, the CDRL1 comprises the sequence as set forth in SEQ ID NO: 751, the CDRL2 comprises the sequence as set forth in SEQ ID NO: 757, and the CDRL3 comprises the sequence as set forth in SEQ ID NO: 753, according to Chothia; or
wherein the CDRH1 comprises the sequence as set forth in SEQ ID NO: 763, the CDRH2 comprises the sequence as set forth in SEQ ID NO: 764, the CDRH3 comprises the sequence as set forth in SEQ ID NO: 765, the CDRL1 comprises the sequence as set forth in SEQ ID NO: 766, the CDRL2 comprises the sequence as set forth in SEQ ID NO: 767, and the CDRL3 comprises the sequence as set forth in SEQ ID NO: 768, according to Contact;
wherein Z082-U5 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 769, the CDRH2 comprises the sequence as set forth in SEQ ID NO: 770, the CDRH3 comprises the sequence as set forth in SEQ ID NO: 771, the CDRL1 comprises the sequence as set forth in SEQ ID NO: 772, the CDRL2 comprises the sequence as set forth in SEQ ID NO: 773, and the CDRL3 comprises the sequence as set forth in SEQ ID NO: 774, according to North;
wherein the CDRH1 comprises the sequence as set forth in SEQ ID NO: 775, the CDRH2 comprises the sequence as set forth in SEQ ID NO: 776, the CDRH3 comprises the sequence as set forth in SEQ ID NO: 777, the CDRL1 comprises the sequence as set forth in SEQ ID NO: 772, the CDRL2 comprises the sequence as set forth in SEQ ID NO: 778, and the CDRL3 comprises the sequence as set forth in SEQ ID NO: 774, according to Kabat;
wherein the CDRH1 comprises the sequence as set forth in SEQ ID NO: 779, the CDRH2 comprises the sequence as set forth in SEQ ID NO: 780, the CDRH3 comprises the sequence as set forth in SEQ ID NO: 771, the CDRL1 comprises the sequence as set forth in SEQ ID NO: 781, the CDRL2 comprises the sequence DD, and the CDRL3 comprises the sequence as set forth in SEQ ID NO: 774 according to IMGT;
wherein the CDRH1 comprises the sequence as set forth in SEQ ID NO: 782, the CDRH2 comprises the sequence as set forth in SEQ ID NO: 783, the CDRH3 comprises the sequence as set forth in SEQ ID NO: 777, the CDRL1 comprises the sequence as set forth in SEQ ID NO: 772, the CDRL2 comprises the sequence as set forth in SEQ ID NO: 778, and the CDRL3 comprises the sequence as set forth in SEQ ID NO: 774, according to Chothia; or
wherein the CDRH1 comprises the sequence as set forth in SEQ ID NO: 784, the CDRH2 comprises the sequence as set forth in SEQ ID NO: 785, the CDRH3 comprises the sequence as set forth in SEQ ID NO: 786, the CDRL1 comprises the sequence as set forth in SEQ ID NO: 745, the CDRL2 comprises the sequence as set forth in SEQ ID NO: 787, and the CDRL3 comprises the sequence as set forth in SEQ ID NO: 788, according to Contact;
wherein Z082-U6 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 789, the CDRH2 comprises the sequence as set forth in SEQ ID NO: 790, the CDRH3 comprises the sequence as set forth in SEQ ID NO: 791, the CDRL1 comprises the sequence as set forth in SEQ ID NO: 792, the CDRL2 comprises the sequence as set forth in SEQ ID NO: 793, and the CDRL3 comprises the sequence as set forth in SEQ ID NO: 794, according to North;
wherein the CDRH1 comprises the sequence as set forth in SEQ ID NO: 795, the CDRH2 comprises the sequence as set forth in SEQ ID NO: 796, the CDRH3 comprises the sequence as set forth in SEQ ID NO: 797, the CDRL1 comprises the sequence as set forth in SEQ ID NO: 792, the CDRL2 comprises the sequence as set forth in SEQ ID NO: 798, and the CDRL3 comprises the sequence as set forth in SEQ ID NO: 794, according to Kabat;
wherein the CDRH1 comprises the sequence as set forth in SEQ ID NO: 799, the CDRH2 comprises the sequence as set forth in SEQ ID NO: 800, the CDRH3 comprises the sequence as set forth in SEQ ID NO: 791, the CDRL1 comprises the sequence as set forth in SEQ ID NO: 801, the CDRL2 comprises the sequence DA, and the CDRL3 comprises the sequence as set forth in SEQ ID NO: 794 according to IMGT;
wherein the CDRH1 comprises the sequence as set forth in SEQ ID NO: 802, the CDRH2 comprises the sequence as set forth in SEQ ID NO: 803, the CDRH3 comprises the sequence as set forth in SEQ ID NO: 797, the CDRL1 comprises the sequence as set forth in SEQ ID NO: 792, the CDRL2 comprises the sequence as set forth in SEQ ID NO: 798, and the CDRL3 comprises the sequence as set forth in SEQ ID NO: 794, according to Chothia; or
wherein the CDRH1 comprises the sequence as set forth in SEQ ID NO: 804, the CDRH2 comprises the sequence as set forth in SEQ ID NO: 805, the CDRH3 comprises the sequence as set forth in SEQ ID NO: 806, the CDRL1 comprises the sequence as set forth in SEQ ID NO: 807, the CDRL2 comprises the sequence as set forth in SEQ ID NO: 808, and the CDRL3 comprises the sequence as set forth in SEQ ID NO: 809, according to Contact;
wherein W056-01 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 810, the CDRH2 comprises the sequence as set forth in SEQ ID NO: 811, the CDRH3 comprises the sequence as set forth in SEQ ID NO: 315, the CDRL1 comprises the sequence as set forth in SEQ ID NO: 812, the CDRL2 comprises the sequence as set forth in SEQ ID NO: 813, and the CDRL3 comprises the sequence as set forth in SEQ ID NO: 814, according to North;
wherein the CDRH1 comprises the sequence as set forth in SEQ ID NO: 319, the CDRH2 comprises the sequence as set forth in SEQ ID NO: 815, the CDRH3 comprises the sequence as set forth in SEQ ID NO: 321, the CDRL1 comprises the sequence as set forth in SEQ ID NO: 812, the CDRL2 comprises the sequence as set forth in SEQ ID NO: 816, and the CDRL3 comprises the sequence as set forth in SEQ ID NO: 814, according to Kabat;
wherein the CDRH1 comprises the sequence as set forth in SEQ ID NO: 817, the CDRH2 comprises the sequence as set forth in SEQ ID NO: 818, the CDRH3 comprises the sequence as set forth in SEQ ID NO: 315, the CDRL1 comprises the sequence as set forth in SEQ ID NO: 819, the CDRL2 comprises the sequence KA, and the CDRL3 comprises the sequence as set forth in SEQ ID NO: 814 according to IMGT;
wherein the CDRH1 comprises the sequence as set forth in SEQ ID NO: 820, the CDRH2 comprises the sequence as set forth in SEQ ID NO: 821, the CDRH3 comprises the sequence as set forth in SEQ ID NO: 321, the CDRL1 comprises the sequence as set forth in SEQ ID NO: 812, the CDRL2 comprises the sequence as set forth in SEQ ID NO: 816, and the CDRL3 comprises the sequence as set forth in SEQ ID NO: 814, according to Chothia; or
wherein the CDRH1 comprises the sequence as set forth in SEQ ID NO: 822, the CDRH2 comprises the sequence as set forth in SEQ ID NO: 823, the CDRH3 comprises the sequence as set forth in SEQ ID NO: 325, the CDRL1 comprises the sequence as set forth in SEQ ID NO: 326, the CDRL2 comprises the sequence as set forth in SEQ ID NO: 824, and the CDRL3 comprises the sequence as set forth in SEQ ID NO: 825, according to Contact;
wherein W056-02 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 826, the CDRH2 comprises the sequence as set forth in SEQ ID NO: 827, the CDRH3 comprises the sequence as set forth in SEQ ID NO: 828, the CDRL1 comprises the sequence as set forth in SEQ ID NO: 829, the CDRL2 comprises the sequence as set forth in SEQ ID NO: 830, and the CDRL3 comprises the sequence as set forth in SEQ ID NO: 831, according to North;
wherein the CDRH1 comprises the sequence as set forth in SEQ ID NO: 319, the CDRH2 comprises the sequence as set forth in SEQ ID NO: 832, the CDRH3 comprises the sequence as set forth in SEQ ID NO: 833, the CDRL1 comprises the sequence as set forth in SEQ ID NO: 829, the CDRL2 comprises the sequence as set forth in SEQ ID NO: 834, and the CDRL3 comprises the sequence as set forth in SEQ ID NO: 831, according to Kabat;
wherein the CDRH1 comprises the sequence as set forth in SEQ ID NO: 835, the CDRH2 comprises the sequence as set forth in SEQ ID NO: 324, the CDRH3 comprises the sequence as set forth in SEQ ID NO: 828, the CDRL1 comprises the sequence as set forth in SEQ ID NO: 836, the CDRL2 comprises the sequence KV, and the CDRL3 comprises the sequence as set forth in SEQ ID NO: 831 according to IMGT;
wherein the CDRH1 comprises the sequence as set forth in SEQ ID NO: 837, the CDRH2 comprises the sequence as set forth in SEQ ID NO: 327, the CDRH3 comprises the sequence as set forth in SEQ ID NO: 833, the CDRL1 comprises the sequence as set forth in SEQ ID NO: 829, the CDRL2 comprises the sequence as set forth in SEQ ID NO: 834, and the CDRL3 comprises the sequence as set forth in SEQ ID NO: 831, according to Chothia; or
wherein the CDRH1 comprises the sequence as set forth in SEQ ID NO: 822, the CDRH2 comprises the sequence as set forth in SEQ ID NO: 838, the CDRH3 comprises the sequence as set forth in SEQ ID NO: 839, the CDRL1 comprises the sequence as set forth in SEQ ID NO: 840, the CDRL2 comprises the sequence as set forth in SEQ ID NO: 841, and the CDRL3 comprises the sequence as set forth in SEQ ID NO: 842, according to Contact;
wherein W917-01 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 843, the CDRH2 comprises the sequence as set forth in SEQ ID NO: 844, the CDRH3 comprises the sequence as set forth in SEQ ID NO: 845, the CDRL1 comprises the sequence as set forth in SEQ ID NO: 295, the CDRL2 comprises the sequence as set forth in SEQ ID NO: 296, and the CDRL3 comprises the sequence as set forth in SEQ ID NO: 846, according to North;
wherein the CDRH1 comprises the sequence as set forth in SEQ ID NO: 847, the CDRH2 comprises the sequence as set forth in SEQ ID NO: 848, the CDRH3 comprises the sequence as set forth in SEQ ID NO: 849, the CDRL1 comprises the sequence as set forth in SEQ ID NO: 295, the CDRL2 comprises the sequence as set forth in SEQ ID NO: 301, and the CDRL3 comprises the sequence as set forth in SEQ ID NO: 846, according to Kabat;
wherein the CDRH1 comprises the sequence as set forth in SEQ ID NO: 850, the CDRH2 comprises the sequence as set forth in SEQ ID NO: 851, the CDRH3 comprises the sequence as set forth in SEQ ID NO: 845, the CDRL1 comprises the sequence as set forth in SEQ ID NO: 304, the CDRL2 comprises the sequence AA, and the CDRL3 comprises the sequence as set forth in SEQ ID NO: 846 according to IMGT;
wherein the CDRH1 comprises the sequence as set forth in SEQ ID NO: 852, the CDRH2 comprises the sequence as set forth in SEQ ID NO: 853, the CDRH3 comprises the sequence as set forth in SEQ ID NO: 849, the CDRL1 comprises the sequence as set forth in SEQ ID NO: 295, the CDRL2 comprises the sequence as set forth in SEQ ID NO: 301, and the CDRL3 comprises the sequence as set forth in SEQ ID NO: 846, according to Chothia; or
wherein the CDRH1 comprises the sequence as set forth in SEQ ID NO: 854, the CDRH2 comprises the sequence as set forth in SEQ ID NO: 855, the CDRH3 comprises the sequence as set forth in SEQ ID NO: 856, the CDRL1 comprises the sequence as set forth in SEQ ID NO: 310, the CDRL2 comprises the sequence as set forth in SEQ ID NO: 857, and the CDRL3 comprises the sequence as set forth in SEQ ID NO: 846, according to Contact;
wherein W917-02 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 858, the CDRH2 comprises the sequence as set forth in SEQ ID NO: 859, the CDRH3 comprises the sequence as set forth in SEQ ID NO: 860, the CDRL1 comprises the sequence as set forth in SEQ ID NO: 861, the CDRL2 comprises the sequence as set forth in SEQ ID NO: 862, and the CDRL3 comprises the sequence as set forth in SEQ ID NO: 863, according to North;
wherein the CDRH1 comprises the sequence as set forth in SEQ ID NO: 864, the CDRH2 comprises the sequence as set forth in SEQ ID NO: 865, the CDRH3 comprises the sequence as set forth in SEQ ID NO: 866, the CDRL1 comprises the sequence as set forth in SEQ ID NO: 861, the CDRL2 comprises the sequence as set forth in SEQ ID NO: 867, and the CDRL3 comprises the sequence as set forth in SEQ ID NO: 863, according to Kabat;
wherein the CDRH1 comprises the sequence as set forth in SEQ ID NO: 864, the CDRH2 comprises the sequence as set forth in SEQ ID NO: 865, the CDRH3 comprises the sequence as set forth in SEQ ID NO: 860, the CDRL1 comprises the sequence as set forth in SEQ ID NO: 866, the CDRL2 comprises the sequence KA, and the CDRL3 comprises the sequence as set forth in SEQ ID NO: 863 according to IMGT;
wherein the CDRH1 comprises the sequence as set forth in SEQ ID NO: 305, the CDRH2 comprises the sequence as set forth in SEQ ID NO: 867, the CDRH3 comprises the sequence as set forth in SEQ ID NO: 866, the CDRL1 comprises the sequence as set forth in SEQ ID NO: 861, the CDRL2 comprises the sequence as set forth in SEQ ID NO: 867, and the CDRL3 comprises the sequence as set forth in SEQ ID NO: 863, according to Chothia; or
wherein the CDRH1 comprises the sequence as set forth in SEQ ID NO: 868, the CDRH2 comprises the sequence as set forth in SEQ ID NO: 869, the CDRH3 comprises the sequence as set forth in SEQ ID NO: 870, the CDRL1 comprises the sequence as set forth in SEQ ID NO: 871, the CDRL2 comprises the sequence as set forth in SEQ ID NO: 872, and the CDRL3 comprises the sequence as set forth in SEQ ID NO: 863, according to Contact;
wherein W917-03 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 873, the CDRH2 comprises the sequence as set forth in SEQ ID NO: 874, the CDRH3 comprises the sequence as set forth in SEQ ID NO: 875, the CDRL1 comprises the sequence as set forth in SEQ ID NO: 360, the CDRL2 comprises the sequence as set forth in SEQ ID NO: 361, and the CDRL3 comprises the sequence as set forth in SEQ ID NO: 876, according to North;
wherein the CDRH1 comprises the sequence as set forth in SEQ ID NO: 877, the CDRH2 comprises the sequence as set forth in SEQ ID NO: 878, the CDRH3 comprises the sequence as set forth in SEQ ID NO: 879, the CDRL1 comprises the sequence as set forth in SEQ ID NO: 360, the CDRL2 comprises the sequence as set forth in SEQ ID NO: 366, and the CDRL3 comprises the sequence as set forth in SEQ ID NO: 876, according to Kabat;
wherein the CDRH1 comprises the sequence as set forth in SEQ ID NO: 880, the CDRH2 comprises the sequence as set forth in SEQ ID NO: 881, the CDRH3 comprises the sequence as set forth in SEQ ID NO: 875, the CDRL1 comprises the sequence as set forth in SEQ ID NO: 369, the CDRL2 comprises the sequence KD, and the CDRL3 comprises the sequence as set forth in SEQ ID NO: 876 according to IMGT;
wherein the CDRH1 comprises the sequence as set forth in SEQ ID NO: 882, the CDRH2 comprises the sequence as set forth in SEQ ID NO: 883, the CDRH3 comprises the sequence as set forth in SEQ ID NO: 879, the CDRL1 comprises the sequence as set forth in SEQ ID NO: 360, the CDRL2 comprises the sequence as set forth in SEQ ID NO: 366, and the CDRL3 comprises the sequence as set forth in SEQ ID NO: 876, according to Chothia; or
wherein the CDRH1 comprises the sequence as set forth in SEQ ID NO: 884, the CDRH2 comprises the sequence as set forth in SEQ ID NO: 885, the CDRH3 comprises the sequence as set forth in SEQ ID NO: 886, the CDRL1 comprises the sequence as set forth in SEQ ID NO: 74, the CDRL2 comprises the sequence as set forth in SEQ ID NO: 375, and the CDRL3 comprises the sequence as set forth in SEQ ID NO: 887, according to Contact;
wherein Z504-01 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 888, the CDRH2 comprises the sequence as set forth in SEQ ID NO: 889, the CDRH3 comprises the sequence as set forth in SEQ ID NO: 890, the CDRL1 comprises the sequence as set forth in SEQ ID NO: 891, the CDRL2 comprises the sequence as set forth in SEQ ID NO: 741, and the CDRL3 comprises the sequence as set forth in SEQ ID NO: 892, according to North;
wherein the CDRH1 comprises the sequence as set forth in SEQ ID NO: 893, the CDRH2 comprises the sequence as set forth in SEQ ID NO: 894, the CDRH3 comprises the sequence as set forth in SEQ ID NO: 895, the CDRL1 comprises the sequence as set forth in SEQ ID NO: 891, the CDRL2 comprises the sequence as set forth in SEQ ID NO: 743, and the CDRL3 comprises the sequence as set forth in SEQ ID NO: 892, according to Kabat;
wherein the CDRH1 comprises the sequence as set forth in SEQ ID NO: 896, the CDRH2 comprises the sequence as set forth in SEQ ID NO: 897, the CDRH3 comprises the sequence as set forth in SEQ ID NO: 890, the CDRL1 comprises the sequence as set forth in SEQ ID NO: 898, the CDRL2 comprises the sequence YD, and the CDRL3 comprises the sequence as set forth in SEQ ID NO: 892 according to IMGT;
wherein the CDRH1 comprises the sequence as set forth in SEQ ID NO: 899, the CDRH2 comprises the sequence as set forth in SEQ ID NO: 900, the CDRH3 comprises the sequence as set forth in SEQ ID NO: 895, the CDRL1 comprises the sequence as set forth in SEQ ID NO: 891, the CDRL2 comprises the sequence as set forth in SEQ ID NO: 743, and the CDRL3 comprises the sequence as set forth in SEQ ID NO: 892, according to Chothia; or
wherein the CDRH1 comprises the sequence as set forth in SEQ ID NO: 901, the CDRH2 comprises the sequence as set forth in SEQ ID NO: 902, the CDRH3 comprises the sequence as set forth in SEQ ID NO: 903, the CDRL1 comprises the sequence as set forth in SEQ ID NO: 745, the CDRL2 comprises the sequence as set forth in SEQ ID NO: 904, and the CDRL3 comprises the sequence as set forth in SEQ ID NO: 905, according to Contact;
wherein Z504-02 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 906, the CDRH2 comprises the sequence as set forth in SEQ ID NO: 907, the CDRH3 comprises the sequence as set forth in SEQ ID NO: 908, the CDRL1 comprises the sequence as set forth in SEQ ID NO: 861, the CDRL2 comprises the sequence as set forth in SEQ ID NO: 909, and the CDRL3 comprises the sequence as set forth in SEQ ID NO: 910, according to North;
wherein the CDRH1 comprises the sequence as set forth in SEQ ID NO: 911, the CDRH2 comprises the sequence as set forth in SEQ ID NO: 912, the CDRH3 comprises the sequence as set forth in SEQ ID NO: 913, the CDRL1 comprises the sequence as set forth in SEQ ID NO: 861, the CDRL2 comprises the sequence as set forth in SEQ ID NO: 914, and the CDRL3 comprises the sequence as set forth in SEQ ID NO: 910, according to Kabat;
wherein the CDRH1 comprises the sequence as set forth in SEQ ID NO: 915, the CDRH2 comprises the sequence as set forth in SEQ ID NO: 916, the CDRH3 comprises the sequence as set forth in SEQ ID NO: 908, the CDRL1 comprises the sequence as set forth in SEQ ID NO: 866, the CDRL2 comprises the sequence KA, and the CDRL3 comprises the sequence as set forth in SEQ ID NO: 910 according to IMGT;
wherein the CDRH1 comprises the sequence as set forth in SEQ ID NO: 917, the CDRH2 comprises the sequence as set forth in SEQ ID NO: 918, the CDRH3 comprises the sequence as set forth in SEQ ID NO: 913, the CDRL1 comprises the sequence as set forth in SEQ ID NO: 861, the CDRL2 comprises the sequence as set forth in SEQ ID NO: 914, and the CDRL3 comprises the sequence as set forth in SEQ ID NO: 910, according to Chothia; or
wherein the CDRH1 comprises the sequence as set forth in SEQ ID NO: 919, the CDRH2 comprises the sequence as set forth in SEQ ID NO: 920, the CDRH3 comprises the sequence as set forth in SEQ ID NO: 921, the CDRL1 comprises the sequence as set forth in SEQ ID NO: 871, the CDRL2 comprises the sequence as set forth in SEQ ID NO: 922, and the CDRL3 comprises the sequence as set forth in SEQ ID NO: 923, according to Contact;
wherein Z538-01 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 924, the CDRH2 comprises the sequence as set forth in SEQ ID NO: 925, the CDRH3 comprises the sequence as set forth in SEQ ID NO: 926, the CDRL1 comprises the sequence as set forth in SEQ ID NO: 927, the CDRL2 comprises the sequence as set forth in SEQ ID NO: 642, and the CDRL3 comprises the sequence as set forth in SEQ ID NO: 928, according to North;
wherein the CDRH1 comprises the sequence as set forth in SEQ ID NO: 929, the CDRH2 comprises the sequence as set forth in SEQ ID NO: 930, the CDRH3 comprises the sequence as set forth in SEQ ID NO: 931, the CDRL1 comprises the sequence as set forth in SEQ ID NO: 927, the CDRL2 comprises the sequence as set forth in SEQ ID NO: 647, and the CDRL3 comprises the sequence as set forth in SEQ ID NO: 928, according to Kabat;
wherein the CDRH1 comprises the sequence as set forth in SEQ ID NO: 932, the CDRH2 comprises the sequence as set forth in SEQ ID NO: 933, the CDRH3 comprises the sequence as set forth in SEQ ID NO: 934, the CDRL1 comprises the sequence as set forth in SEQ ID NO: 935, the CDRL2 comprises the sequence AA, and the CDRL3 comprises the sequence as set forth in SEQ ID NO: 928 according to IMGT;
wherein the CDRH1 comprises the sequence as set forth in SEQ ID NO: 936, the CDRH2 comprises the sequence as set forth in SEQ ID NO: 937, the CDRH3 comprises the sequence as set forth in SEQ ID NO: 931, the CDRL1 comprises the sequence as set forth in SEQ ID NO: 927, the CDRL2 comprises the sequence as set forth in SEQ ID NO: 647, and the CDRL3 comprises the sequence as set forth in SEQ ID NO: 928, according to Chothia; or
wherein the CDRH1 comprises the sequence as set forth in SEQ ID NO: 938, the CDRH2 comprises the sequence as set forth in SEQ ID NO: 939, the CDRH3 comprises the sequence as set forth in SEQ ID NO: 940, the CDRL1 comprises the sequence as set forth in SEQ ID NO: 941, the CDRL2 comprises the sequence as set forth in SEQ ID NO: 942, and the CDRL3 comprises the sequence as set forth in SEQ ID NO: 943, according to Contact;
wherein Z538-02 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 944, the CDRH2 comprises the sequence as set forth in SEQ ID NO: 945, the CDRH3 comprises the sequence as set forth in SEQ ID NO: 946, the CDRL1 comprises the sequence as set forth in SEQ ID NO: 947, the CDRL2 comprises the sequence as set forth in SEQ ID NO: 948, and the CDRL3 comprises the sequence as set forth in SEQ ID NO: 949, according to North;
wherein the CDRH1 comprises the sequence as set forth in SEQ ID NO: 950, the CDRH2 comprises the sequence as set forth in SEQ ID NO: 951, the CDRH3 comprises the sequence as set forth in SEQ ID NO: 952, the CDRL1 comprises the sequence as set forth in SEQ ID NO: 947, the CDRL2 comprises the sequence as set forth in SEQ ID NO: 953, and the CDRL3 comprises the sequence as set forth in SEQ ID NO: 949, according to Kabat;
wherein the CDRH1 comprises the sequence as set forth in SEQ ID NO: 954, the CDRH2 comprises the sequence as set forth in SEQ ID NO: 955, the CDRH3 comprises the sequence as set forth in SEQ ID NO: 946, the CDRL1 comprises the sequence as set forth in SEQ ID NO: 956, the CDRL2 comprises the sequence DV, and the CDRL3 comprises the sequence as set forth in SEQ ID NO: 949 according to IMGT;
wherein the CDRH1 comprises the sequence as set forth in SEQ ID NO: 957, the CDRH2 comprises the sequence as set forth in SEQ ID NO: 958, the CDRH3 comprises the sequence as set forth in SEQ ID NO: 952, the CDRL1 comprises the sequence as set forth in SEQ ID NO: 947, the CDRL2 comprises the sequence as set forth in SEQ ID NO: 953, and the CDRL3 comprises the sequence as set forth in SEQ ID NO: 949, according to Chothia; or
wherein the CDRH1 comprises the sequence as set forth in SEQ ID NO: 959, the CDRH2 comprises the sequence as set forth in SEQ ID NO: 960, the CDRH3 comprises the sequence as set forth in SEQ ID NO: 961, the CDRL1 comprises the sequence as set forth in SEQ ID NO: 962, the CDRL2 comprises the sequence as set forth in SEQ ID NO: 963, and the CDRL3 comprises the sequence as set forth in SEQ ID NO: 949, according to Contact;
wherein Z573-01 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 964, the CDRH2 comprises the sequence as set forth in SEQ ID NO: 827, the CDRH3 comprises the sequence as set forth in SEQ ID NO: 965, the CDRL1 comprises the sequence as set forth in SEQ ID NO: 966, the CDRL2 comprises the sequence as set forth in SEQ ID NO: 967, and the CDRL3 comprises the sequence as set forth in SEQ ID NO: 968, according to North;
wherein the CDRH1 comprises the sequence as set forth in SEQ ID NO: 969, the CDRH2 comprises the sequence as set forth in SEQ ID NO: 832, the CDRH3 comprises the sequence as set forth in SEQ ID NO: 970, the CDRL1 comprises the sequence as set forth in SEQ ID NO: 966, the CDRL2 comprises the sequence as set forth in SEQ ID NO: 971, and the CDRL3 comprises the sequence as set forth in SEQ ID NO: 968, according to Kabat;
wherein the CDRH1 comprises the sequence as set forth in SEQ ID NO: 972, the CDRH2 comprises the sequence as set forth in SEQ ID NO: 324, the CDRH3 comprises the sequence as set forth in SEQ ID NO: 965, the CDRL1 comprises the sequence as set forth in SEQ ID NO: 973, the CDRL2 comprises the sequence WA, and the CDRL3 comprises the sequence as set forth in SEQ ID NO: 968 according to IMGT;
wherein the CDRH1 comprises the sequence as set forth in SEQ ID NO: 974, the CDRH2 comprises the sequence as set forth in SEQ ID NO: 327, the CDRH3 comprises the sequence as set forth in SEQ ID NO: 970, the CDRL1 comprises the sequence as set forth in SEQ ID NO: 966, the CDRL2 comprises the sequence as set forth in SEQ ID NO: 971, and the CDRL3 comprises the sequence as set forth in SEQ ID NO: 968, according to Chothia; or
wherein the CDRH1 comprises the sequence as set forth in SEQ ID NO: 975, the CDRH2 comprises the sequence as set forth in SEQ ID NO: 838, the CDRH3 comprises the sequence as set forth in SEQ ID NO: 976, the CDRL1 comprises the sequence as set forth in SEQ ID NO: 977, the CDRL2 comprises the sequence as set forth in SEQ ID NO: 978, and the CDRL3 comprises the sequence as set forth in SEQ ID NO: 979, according to Contact; or
wherein Z573-02 comprises a heavy chain variable region comprising a CDRH1, CDRH2, and CDRH3 and a light chain variable region comprising a CDRL1, CDRL2, and CDRL3;
wherein the CDRH1 comprises the sequence as set forth in SEQ ID NO: 980, the CDRH2 comprises the sequence as set forth in SEQ ID NO: 981, the CDRH3 comprises the sequence as set forth in SEQ ID NO: 683, the CDRL1 comprises the sequence as set forth in SEQ ID NO: 68, the CDRL2 comprises the sequence as set forth in SEQ ID NO: 361, and the CDRL3 comprises the sequence as set forth in SEQ ID NO: 982, according to North;
wherein the CDRH1 comprises the sequence as set forth in SEQ ID NO: 983, the CDRH2 comprises the sequence as set forth in SEQ ID NO: 984, the CDRH3 comprises the sequence as set forth in SEQ ID NO: 688, the CDRL1 comprises the sequence as set forth in SEQ ID NO: 684, the CDRL2 comprises the sequence as set forth in SEQ ID NO: 366, and the CDRL3 comprises the sequence as set forth in SEQ ID NO: 982, according to Kabat;
wherein the CDRH1 comprises the sequence as set forth in SEQ ID NO: 985, the CDRH2 comprises the sequence as set forth in SEQ ID NO: 986, the CDRH3 comprises the sequence as set forth in SEQ ID NO: 683, the CDRL1 comprises the sequence as set forth in SEQ ID NO: 691, the CDRL2 comprises the sequence KD, and the CDRL3 comprises the sequence as set forth in SEQ ID NO: 982 according to IMGT;
wherein the CDRH1 comprises the sequence as set forth in SEQ ID NO: 987, the CDRH2 comprises the sequence as set forth in SEQ ID NO: 988, the CDRH3 comprises the sequence as set forth in SEQ ID NO: 688, the CDRL1 comprises the sequence as set forth in SEQ ID NO: 684, the CDRL2 comprises the sequence as set forth in SEQ ID NO: 366, and the CDRL3 comprises the sequence as set forth in SEQ ID NO: 982, according to Chothia; or
wherein the CDRH1 comprises the sequence as set forth in SEQ ID NO: 989, the CDRH2 comprises the sequence as set forth in SEQ ID NO: 990, the CDRH3 comprises the sequence as set forth in SEQ ID NO: 696, the CDRL1 comprises the sequence as set forth in SEQ ID NO: 697, the CDRL2 comprises the sequence as set forth in SEQ ID NO: 375, and the CDRL3 comprises the sequence as set forth in SEQ ID NO: 982, according to Contact.

204. The antibody or fragment thereof of claim 203,
wherein the CDRH1 is encoded by the sequence as set forth in SEQ ID NO: 991, the CDRH2 is encoded by the sequence as set forth in SEQ ID NO: 992, the CDRH3 is encoded by the sequence as set forth in SEQ ID NO: 993, the CDRL1 is encoded by the sequence as set forth in SEQ ID NO: 994, the CDRL2 is encoded by the sequence GGTAACAGT, the CDRL3 is encoded by the sequence as set forth in SEQ ID NO: 996;
wherein the CDRH1 is encoded by the sequence as set forth in SEQ ID NO: 997, the CDRH2 is encoded by the sequence as set forth in SEQ ID NO: 998, the CDRH3 is encoded by the sequence as set forth in SEQ ID NO: 999, the CDRL1 is encoded by the sequence as set forth in SEQ ID NO: 1000, the CDRL2 is encoded by the sequence GAGGTCAGT, the CDRL3 is encoded by the sequence as set forth in SEQ ID NO: 1002;
wherein the CDRH1 is encoded by the sequence as set forth in SEQ ID NO: 1003, the CDRH2 is encoded by the sequence as set forth in SEQ ID NO: 1004, the CDRH3 is encoded by the sequence as set forth in SEQ ID NO: 1005, the CDRL1 is encoded by the sequence as set forth in SEQ ID NO: 1006, the CDRL2 is encoded by the sequence GCTGCATCC, the CDRL3 is encoded by the sequence as set forth in SEQ ID NO: 1008;
wherein the CDRH1 is encoded by the sequence as set forth in SEQ ID NO: 1009, the CDRH2 is encoded by the sequence as set forth in SEQ ID NO: 1010, the CDRH3 is encoded by the sequence as set forth in SEQ ID NO: 1111, the CDRL1 is encoded by the sequence as set forth in SEQ ID NO: 1112, the CDRL2 is encoded by the sequence GAGGCGTCC, the CDRL3 is encoded by the sequence as set forth in SEQ ID NO: 1114;
wherein the CDRH1 is encoded by the sequence as set forth in SEQ ID NO: 997, the CDRH2 is encoded by the sequence as set forth in SEQ ID NO: 998, the CDRH3 is encoded by the sequence as set forth in SEQ ID NO: 999, the CDRL1 is encoded by the sequence as set forth in SEQ ID NO: 1000, the CDRL2 is encoded by the sequence GAGGTCAGT, the CDRL3 is encoded by the sequence as set forth in SEQ ID NO: 1002;
wherein the CDRH1 is encoded by the sequence as set forth in SEQ ID NO: 1115, the CDRH2 is encoded by the sequence as set forth in SEQ ID NO: 1116, the CDRH3 is encoded by the sequence as set forth in SEQ ID NO: 1111, the CDRL1 is encoded by the sequence as set forth in SEQ ID NO: 1117, the CDRL2 is encoded by the sequence GAGGCGTCC, the CDRL3 is encoded by the sequence as set forth in SEQ ID NO: 1118;
wherein the CDRH1 is encoded by the sequence as set forth in SEQ ID NO: 1119, the CDRH2 is encoded by the sequence as set forth in SEQ ID NO: 1120, the CDRH3 is encoded by the sequence as set forth in SEQ ID NO: 1121, the CDRL1 is encoded by the sequence as set forth in SEQ ID NO: 1122, the CDRL2 is encoded by the sequence GCTGCATCC, the CDRL3 is encoded by the sequence as set forth in SEQ ID NO: 1123;
wherein the CDRH1 is encoded by the sequence as set forth in SEQ ID NO: 1124, the CDRH2 is encoded by the sequence as set forth in SEQ ID NO: 1125, the CDRH3 is encoded by the sequence as set forth in SEQ ID NO: 1126, the CDRL1 is encoded by the sequence as set forth in SEQ ID NO: 1127, the CDRL2 is encoded by the sequence AAAGACAGT, the CDRL3 is encoded by the sequence as set forth in SEQ ID NO: 1129;
wherein the CDRH1 is encoded by the sequence as set forth in SEQ ID NO: 1124, the CDRH2 is encoded by the sequence as set forth in SEQ ID NO: 1125, the CDRH3 is encoded by the sequence as set forth in SEQ ID NO: 1126, the CDRL1 is encoded by the sequence as set forth in SEQ ID NO: 1130, the CDRL2 is encoded by the sequence as set forth in SEQ ID NO: 1131, the CDRL3 is encoded by the sequence as set forth in SEQ ID NO: 1132;
wherein the CDRH1 is encoded by the sequence as set forth in SEQ ID NO: 1133, the CDRH2 is encoded by the sequence as set forth in SEQ ID NO: 1134, the CDRH3 is encoded by the sequence as set forth in SEQ ID NO: 1135, the CDRL1 is encoded by the sequence as set forth in SEQ ID NO: 1136, the CDRL2 is encoded by the sequence AAAGACAAT, the CDRL3 is encoded by the sequence as set forth in SEQ ID NO: 1138;
wherein the CDRH1 is encoded by the sequence as set forth in SEQ ID NO: 1139, the CDRH2 is encoded by the sequence as set forth in SEQ ID NO: 1140, the CDRH3 is encoded by the sequence as set forth in SEQ ID NO: 1141, the CDRL1 is encoded by the sequence as set forth in SEQ ID NO: 1142, the CDRL2 is encoded by the sequence GGTGCATCC, the CDRL3 is encoded by the sequence as set forth in SEQ ID NO: 1144;
wherein the CDRH1 is encoded by the sequence as set forth in SEQ ID NO: 1145, the CDRH2 is encoded by the sequence as set forth in SEQ ID NO: 1146, the CDRH3 is encoded by the sequence as set forth in SEQ ID NO: 1147, the CDRL1 is encoded by the sequence as set forth in SEQ ID NO: 1148, the CDRL2 is encoded by the sequence GATGTCACT, the CDRL3 is encoded by the sequence as set forth in SEQ ID NO: 1150;
wherein the CDRH1 is encoded by the sequence as set forth in SEQ ID NO: 1151, the CDRH2 is encoded by the sequence as set forth in SEQ ID NO: 1152, the CDRH3 is encoded by the sequence as set forth in SEQ ID NO: 1153, the CDRL1 is encoded by the sequence as set forth in SEQ ID NO: 1154, the CDRL2 is encoded by the sequence AAGGACAGC, the CDRL3 is encoded by the sequence as set forth in SEQ ID NO: 1156;
wherein the CDRH1 is encoded by the sequence as set forth in SEQ ID NO: 1157, the CDRH2 is encoded by the sequence as set forth in SEQ ID NO: 1158, the CDRH3 is encoded by the sequence as set forth in SEQ ID NO: 1159, the CDRL1 is encoded by the sequence as set forth in SEQ ID NO: 1160, the CDRL2 is encoded by the sequence AAAGACACT, the CDRL3 is encoded by the sequence as set forth in SEQ ID NO: 1162;
wherein the CDRH1 is encoded by the sequence as set forth in SEQ ID NO: 1139, the CDRH2 is encoded by the sequence as set forth in SEQ ID NO: 1140, the CDRH3 is encoded by the sequence as set forth in SEQ ID NO: 1141, the CDRL1 is encoded by the sequence as set forth in SEQ ID NO: 1142, the CDRL2 is encoded by the sequence GGTGCATCC, the CDRL3 is encoded by the sequence as set forth in SEQ ID NO: 1144;
wherein the CDRH1 is encoded by the sequence as set forth in SEQ ID NO: 1163, the CDRH2 is encoded by the sequence as set forth in SEQ ID NO: 1164, the CDRH3 is encoded by the sequence as set forth in SEQ ID NO: 1165, the CDRL1 is encoded by the sequence as set forth in SEQ ID NO: 1166, the CDRL2 is encoded by the sequence AGTGCATCC, the CDRL3 is encoded by the sequence as set forth in SEQ ID NO: 1168;
wherein the CDRH1 is encoded by the sequence as set forth in SEQ ID NO: 1169, the CDRH2 is encoded by the sequence as set forth in SEQ ID NO: 1170, the CDRH3 is encoded by the sequence as set forth in SEQ ID NO: 1171, the CDRL1 is encoded by the sequence as set forth in SEQ ID NO: 1172, the CDRL2 is encoded by the sequence ACTGCGTCC, the CDRL3 is encoded by the sequence as set forth in SEQ ID NO: 1174;
wherein the CDRH1 is encoded by the sequence as set forth in SEQ ID NO: 1175, the CDRH2 is encoded by the sequence as set forth in SEQ ID NO: 998, the CDRH3 is encoded by the sequence as set forth in SEQ ID NO: 1176, the CDRL1 is encoded by the sequence as set forth in SEQ ID NO: 1177, the CDRL2 is encoded by the sequence AGTGCATCC, the CDRL3 is encoded by the sequence as set forth in SEQ ID NO: 1178;
wherein the CDRH1 is encoded by the sequence as set forth in SEQ ID NO: 1179, the CDRH2 is encoded by the sequence as set forth in SEQ ID NO: 1180, the CDRH3 is encoded by the sequence as set forth in SEQ ID NO: 1181, the CDRL1 is encoded by the sequence as set forth in SEQ ID NO: 1182, the CDRL2 is encoded by the sequence GGTGCATCC, the CDRL3 is encoded by the sequence as set forth in SEQ ID NO: 1183;
wherein the CDRH1 is encoded by the sequence as set forth in SEQ ID NO: 1184, the CDRH2 is encoded by the sequence as set forth in SEQ ID NO: 1185, the CDRH3 is encoded by the sequence as set forth in SEQ ID NO: 1186, the CDRL1 is encoded by the sequence as set forth in SEQ ID NO: 1187, the CDRL2 is encoded by the sequence GGTGCATCC, the CDRL3 is encoded by the sequence as set forth in SEQ ID NO: 1188;
wherein the CDRH1 is encoded by the sequence as set forth in SEQ ID NO: 1189, the CDRH2 is encoded by the sequence as set forth in SEQ ID NO: 1185, the CDRH3 is encoded by the sequence as set forth in SEQ ID NO: 1190, the CDRL1 is encoded by the sequence as set forth in SEQ ID NO: 1187, the CDRL2 is encoded by the sequence GGTGCATCC, the CDRL3 is encoded by the sequence as set forth in SEQ ID NO: 1191;
wherein the CDRH1 is encoded by the sequence as set forth in SEQ ID NO: 1192, the CDRH2 is encoded by the sequence as set forth in SEQ ID NO: 1193, the CDRH3 is encoded by the sequence as set forth in SEQ ID NO: 1194, the CDRL1 is encoded by the sequence as set forth in SEQ ID NO: 1195, the CDRL2 is encoded by the sequence ACTACATCC, the CDRL3 is encoded by the sequence as set forth in SEQ ID NO: 1197;
wherein the CDRH1 is encoded by the sequence as set forth in SEQ ID NO: 1198, the CDRH2 is encoded by the sequence as set forth in SEQ ID NO: 1199, the CDRH3 is encoded by the sequence as set forth in SEQ ID NO: 1200, the CDRL1 is encoded by the sequence as set forth in SEQ ID NO: 1201, the CDRL2 is encoded by the sequence as set forth in SEQ ID NO: 1201, the CDRL3 is encoded by the sequence GTTGCATCC;
wherein the CDRH1 is encoded by the sequence as set forth in SEQ ID NO: 1204, the CDRH2 is encoded by the sequence as set forth in SEQ ID NO: 1205, the CDRH3 is encoded by the sequence as set forth in SEQ ID NO: 1206, the CDRL1 is encoded by the sequence as set forth in SEQ ID NO: 1207, the CDRL2 is encoded by the sequence GCTGCTTCC, the CDRL3 is encoded by the sequence as set forth in SEQ ID NO: 1209;
wherein the CDRH1 is encoded by the sequence as set forth in SEQ ID NO: 1210, the CDRH2 is encoded by the sequence as set forth in SEQ ID NO: 1211, the CDRH3 is encoded by the sequence as set forth in SEQ ID NO: 1212, the CDRL1 is encoded by the sequence as set forth in SEQ ID NO: 1213, the CDRL2 is encoded by the sequence as set forth in SEQ ID NO: 1214, the CDRL3 is encoded by the sequence as set forth in SEQ ID NO: 1215;
wherein the CDRH1 is encoded by the sequence as set forth in SEQ ID NO: 1216, the CDRH2 is encoded by the sequence as set forth in SEQ ID NO: 1217, the CDRH3 is encoded by the sequence as set forth in SEQ ID NO: 1218, the CDRL1 is encoded by the sequence as set forth in SEQ ID NO: 1219, the CDRL2 is encoded by the sequence as set forth in SEQ ID NO: 1220, the CDRL3 is encoded by the sequence as set forth in SEQ ID NO: 1221;
wherein the CDRH1 is encoded by the sequence as set forth in SEQ ID NO: 1221, the CDRH2 is encoded by the sequence as set forth in SEQ ID NO: 1222, the CDRH3 is encoded by the sequence as set forth in SEQ ID NO: 1223, the CDRL1 is encoded by the sequence as set forth in SEQ ID NO: 1224, the CDRL2 is encoded by the sequence GGTGCATCC, the CDRL3 is encoded by the sequence as set forth in SEQ ID NO: 1225;
wherein the CDRH1 is encoded by the sequence as set forth in SEQ ID NO: 1226, the CDRH2 is encoded by the sequence as set forth in SEQ ID NO: 1227, the CDRH3 is encoded by the sequence as set forth in SEQ ID NO: 1228, the CDRL1 is encoded by the sequence as set forth in SEQ ID NO: 1229, the CDRL2 is encoded by the sequence GAGGATAAC, the CDRL3 is encoded by the sequence as set forth in SEQ ID NO: 1231;
wherein the CDRH1 is encoded by the sequence as set forth in SEQ ID NO: 1226, the CDRH2 is encoded by the sequence as set forth in SEQ ID NO: 1227, the CDRH3 is encoded by the sequence as set forth in SEQ ID NO: 1228, the CDRL1 is encoded by the sequence as set forth in SEQ ID NO: 1232, the CDRL2 is encoded by the sequence TATGATAGC, the CDRL3 is encoded by the sequence as set forth in SEQ ID NO: 1234;
wherein the CDRH1 is encoded by the sequence as set forth in SEQ ID NO: 1235, the CDRH2 is encoded by the sequence as set forth in SEQ ID NO: 1236, the CDRH3 is encoded by the sequence as set forth in SEQ ID NO: 1237, the CDRL1 is encoded by the sequence as set forth in SEQ ID NO: 1238, the CDRL2 is encoded by the sequence GATGTCACT, the CDRL3 is encoded by the sequence as set forth in SEQ ID NO: 1239;
wherein the CDRH1 is encoded by the sequence as set forth in SEQ ID NO: 1240, the CDRH2 is encoded by the sequence as set forth in SEQ ID NO: 1241, the CDRH3 is encoded by the sequence as set forth in SEQ ID NO: 1242, the CDRL1 is encoded by the sequence as set forth in SEQ ID NO: 1243, the CDRL2 is encoded by the sequence GATGATAGT, the CDRL3 is encoded by the sequence as set forth in SEQ ID NO: 1245;
wherein the CDRH1 is encoded by the sequence as set forth in SEQ ID NO: 1246, the CDRH2 is encoded by the sequence as set forth in SEQ ID NO: 1247, the CDRH3 is encoded by the sequence as set forth in SEQ ID NO: 1248, the CDRL1 is encoded by the sequence as set forth in SEQ ID NO: 1249, the CDRL2 is encoded by the sequence GATGCATCC, the CDRL3 is encoded by the sequence as set forth in SEQ ID NO: 1251;
wherein the CDRH1 is encoded by the sequence as set forth in SEQ ID NO: 1252, the CDRH2 is encoded by the sequence as set forth in SEQ ID NO: 1253, the CDRH3 is encoded by the sequence as set forth in SEQ ID NO: 1254, the CDRL1 is encoded by the sequence as set forth in SEQ ID NO: 1255, the CDRL2 is encoded by the sequence AAGGCGTCT, the CDRL3 is encoded by the sequence as set forth in SEQ ID NO: 1257;
wherein the CDRH1 is encoded by the sequence as set forth in SEQ ID NO: 1258, the CDRH2 is encoded by the sequence as set forth in SEQ ID NO: 1259, the CDRH3 is encoded by the sequence as set forth in SEQ ID NO: 1260, the CDRL1 is encoded by the sequence as set forth in SEQ ID NO: 1261, the CDRL2 is encoded by the sequence AAAGTTTCC, the CDRL3 is encoded by the sequence as set forth in SEQ ID NO: 1263;
wherein the CDRH1 is encoded by the sequence as set forth in SEQ ID NO: 1264, the CDRH2 is encoded by the sequence as set forth in SEQ ID NO: 1265, the CDRH3 is encoded by the sequence as set forth in SEQ ID NO: 1266, the CDRL1 is encoded by the sequence as set forth in SEQ ID NO: 1267, the CDRL2 is encoded by the sequence GCTGCATCC, the CDRL3 is encoded by the sequence as set forth in SEQ ID NO: 1268;
wherein the CDRH1 is encoded by the sequence as set forth in SEQ ID NO: 1269, the CDRH2 is encoded by the sequence as set forth in SEQ ID NO: 1270, the CDRH3 is encoded by the sequence as set forth in SEQ ID NO: 1271, the CDRL1 is encoded by the sequence as set forth in SEQ ID NO: 1272, the CDRL2 is encoded by the sequence AAGGCGTCT, the CDRL3 is encoded by the sequence as set forth in SEQ ID NO: 1273;
wherein the CDRH1 is encoded by the sequence as set forth in SEQ ID NO: 1274, the CDRH2 is encoded by the sequence as set forth in SEQ ID NO: 1275, the CDRH3 is encoded by the sequence as set forth in SEQ ID NO: 1276, the CDRL1 is encoded by the sequence as set forth in SEQ ID NO: 1127, the CDRL2 is encoded by the sequence AAAGACAGT, the CDRL3 is encoded by the sequence as set forth in SEQ ID NO: 1277;
wherein the CDRH1 is encoded by the sequence as set forth in SEQ ID NO: 1278, the CDRH2 is encoded by the sequence as set forth in SEQ ID NO: 1279, the CDRH3 is encoded by the sequence as set forth in SEQ ID NO: 1280, the CDRL1 is encoded by the sequence as set forth in SEQ ID NO: 1281, the CDRL2 is encoded by the sequence TATGATAGC, the CDRL3 is encoded by the sequence as set forth in SEQ ID NO: 1282;
wherein the CDRH1 is encoded by the sequence as set forth in SEQ ID NO: 1283, the CDRH2 is encoded by the sequence as set forth in SEQ ID NO: 1284, the CDRH3 is encoded by the sequence as set forth in SEQ ID NO: 1285, the CDRL1 is encoded by the sequence as set forth in SEQ ID NO: 1272, the CDRL2 is encoded by the sequence AAGGCGTCT, the CDRL3 is encoded by the sequence as set forth in SEQ ID NO: 1286;
wherein the CDRH1 is encoded by the sequence as set forth in SEQ ID NO: 1287, the CDRH2 is encoded by the sequence as set forth in SEQ ID NO: 1288, the CDRH3 is encoded by the sequence as set forth in SEQ ID NO: 1289, the CDRL1 is encoded by the sequence as set forth in SEQ ID NO: 1290, the CDRL2 is encoded by the sequence GCTGCATCC, the CDRL3 is encoded by the sequence as set forth in SEQ ID NO: 1291;
wherein the CDRH1 is encoded by the sequence as set forth in SEQ ID NO: 1292, the CDRH2 is encoded by the sequence as set forth in SEQ ID NO: 1293, the CDRH3 is encoded by the sequence as set forth in SEQ ID NO: 1294, the CDRL1 is encoded by the sequence as set forth in SEQ ID NO: 1295, the CDRL2 is encoded by the sequence GATGTCTCT, the CDRL3 is encoded by the sequence as set forth in SEQ ID NO: 1297;
wherein the CDRH1 is encoded by the sequence as set forth in SEQ ID NO: 1298, the CDRH2 is encoded by the sequence as set forth in SEQ ID NO: 1299, the CDRH3 is encoded by the sequence as set forth in SEQ ID NO: 1300, the CDRL1 is encoded by the sequence as set forth in SEQ ID NO: 1301, the CDRL2 is encoded by the sequence TGGGCTTCT, the CDRL3 is encoded by the sequence as set forth in SEQ ID NO: 1303; or
wherein the CDRH1 is encoded by the sequence as set forth in SEQ ID NO: 1304, the CDRH2 is encoded by the sequence as set forth in SEQ ID NO: 1305, the CDRH3 is encoded by the sequence as set forth in SEQ ID NO: 1306, the CDRL1 is encoded by the sequence as set forth in SEQ ID NO: 1219, the CDRL2 is encoded by the sequence AAAGACAGT, the CDRL3 is encoded by the sequence as set forth in SEQ ID NO: 1307.

205. The antibody or fragment thereof of claim 203,
wherein the heavy chain variable region comprises the sequence as set forth in SEQ ID NO: 47 and the light chain variable region comprises the sequence as set forth in SEQ ID NO: 48; or the heavy chain variable region comprises a sequence having at least 90% sequence identity to the sequence as set forth in SEQ ID NO: 47 and the light chain variable region comprises a sequence having at least 90% sequence identity to the sequence as set forth in SEQ ID NO: 48;
wherein the heavy chain variable region comprises a sequence as set forth in SEQ ID NO: 49 and the light chain variable region comprises the sequence as set forth in SEQ ID NO: 50; or the heavy chain variable region comprises a sequence having at least 90% sequence identity to the sequence as set forth in SEQ ID NO: 49 and the light chain variable region comprises a sequence having at least 90% sequence identity to the sequence as set forth in SEQ ID NO: 50;
wherein the heavy chain variable region comprises the sequence as set forth in SEQ ID NO: 51 and the light chain variable region comprises the sequence as set forth in SEQ ID NO: 52; or the heavy chain variable region comprises a sequence having at least 90% sequence identity to the sequence as set forth in SEQ ID NO: 51 and the light chain variable region comprises a sequence having at least 90% sequence identity to the sequence as set forth in SEQ ID NO: 52;
wherein the heavy chain variable region comprises a sequence as set forth in SEQ ID NO: 53 and the light chain variable region comprises the sequence as set forth in SEQ ID NO: 54; or the heavy chain variable region comprises a sequence having at least 90% sequence identity to the sequence as set forth in SEQ ID NO: 53 and the light chain variable region comprises a sequence having at least 90% sequence identity to the sequence as set forth in SEQ ID NO: 54;
wherein the heavy chain variable region comprises a sequence as set forth in SEQ ID NO: 55 and the light chain variable region comprises the sequence as set forth in SEQ ID NO: 56; or the heavy chain variable region comprises a sequence having at least 90% sequence identity to the sequence as set forth in SEQ ID NO: 55 and the light chain variable region comprises a sequence having at least 90% sequence identity to the sequence as set forth in SEQ ID NO: 56;
wherein the heavy chain variable region comprises a sequence as set forth in SEQ ID NO: 169 and the light chain variable region comprises the sequence as set forth in SEQ ID NO: 170; or the heavy chain variable region comprises a sequence having at least 90% sequence identity to the sequence as set forth in SEQ ID NO: 169 and the light chain variable region comprises a sequence having at least 90% sequence identity to the sequence as set forth in SEQ ID NO: 170;
wherein the heavy chain variable region comprises a sequence as set forth in SEQ ID NO: 171 and the light chain variable region comprises the sequence as set forth in SEQ ID NO: 172; or the heavy chain variable region comprises a sequence having at least 90% sequence identity to the sequence as set forth in SEQ ID NO: 171 and the light chain variable region comprises a sequence having at least 90% sequence identity to the sequence as set forth in SEQ ID NO: 172;
wherein the heavy chain variable region comprises a sequence as set forth in SEQ ID NO: 173 and the light chain variable region comprises the sequence as set forth in SEQ ID NO: 174; or the heavy chain variable region comprises a sequence having at least 90% sequence identity to the sequence as set forth in SEQ ID NO: 173 and the light chain variable region comprises a sequence having at least 90% sequence identity to the sequence as set forth in SEQ ID NO: 174;
wherein the heavy chain variable region comprises a sequence as set forth in SEQ ID NO: 175 and the light chain variable region comprises the sequence as set forth in SEQ ID NO: 176; or the heavy chain variable region comprises a sequence having at least 90% sequence identity to the sequence as set forth in SEQ ID NO: 175 and the light chain variable region comprises a sequence having at least 90% sequence identity to the sequence as set forth in SEQ ID NO: 176;
wherein the heavy chain variable region comprises a sequence as set forth in SEQ ID NO: 177 and the light chain variable region comprises the sequence as set forth in SEQ ID NO: 178; or the heavy chain variable region comprises a sequence having at least 90% sequence identity to the sequence as set forth in SEQ ID NO: 177 and the light chain variable region comprises a sequence having at least 90% sequence identity to the sequence as set forth in SEQ ID NO: 178;
wherein the heavy chain variable region comprises a sequence as set forth in SEQ ID NO: 179 and the light chain variable region comprises the sequence as set forth in SEQ ID NO: 180; or the heavy chain variable region comprises a sequence having at least 90% sequence identity to the sequence as set forth in SEQ ID NO: 179 and the light chain variable region comprises a sequence having at least 90% sequence identity to the sequence as set forth in SEQ ID NO: 180;
wherein the heavy chain variable region comprises a sequence as set forth in SEQ ID NO: 181 and the light chain variable region comprises the sequence as set forth in SEQ ID NO: 182; or the heavy chain variable region comprises a sequence having at least 90% sequence identity to the sequence as set forth in SEQ ID NO: 181 and the light chain variable region comprises a sequence having at least 90% sequence identity to the sequence as set forth in SEQ ID NO: 182;
wherein the heavy chain variable region comprises a sequence as set forth in SEQ ID NO: 183 and the light chain variable region comprises the sequence as set forth in SEQ ID NO: 184; or the heavy chain variable region comprises a sequence having at least 90% sequence identity to the sequence as set forth in SEQ ID NO: 183 and the light chain variable region comprises a sequence having at least 90% sequence identity to the sequence as set forth in SEQ ID NO: 184;
wherein the heavy chain variable region comprises a sequence as set forth in SEQ ID NO: 185 and the light chain variable region comprises the sequence as set forth in SEQ ID NO: 186; or the heavy chain variable region comprises a sequence having at least 90% sequence identity to the sequence as set forth in SEQ ID NO: 185 and the light chain variable region comprises a sequence having at least 90% sequence identity to the sequence as set forth in SEQ ID NO: 186;
wherein the heavy chain variable region comprises a sequence as set forth in SEQ ID NO: 187 and the light chain variable region comprises the sequence as set forth in SEQ ID NO: 188; or the heavy chain variable region comprises a sequence having at least 90% sequence identity to the sequence as set forth in SEQ ID NO: 187 and the light chain variable region comprises a sequence having at least 90% sequence identity to the sequence as set forth in SEQ ID NO: 188;
wherein the heavy chain variable region comprises a sequence as set forth in SEQ ID NO: 189 and the light chain variable region comprises the sequence as set forth in SEQ ID NO: 190; or the heavy chain variable region comprises a sequence having at least 90% sequence identity to the sequence as set forth in SEQ ID NO: 189 and the light chain variable region comprises a sequence having at least 90% sequence identity to the sequence as set forth in SEQ ID NO: 190;
wherein the heavy chain variable region comprises a sequence as set forth in SEQ ID NO: 191 and the light chain variable region comprises the sequence as set forth in SEQ ID NO: 192; or the heavy chain variable region comprises a sequence having at least 90% sequence identity to the sequence as set forth in SEQ ID NO: 191 and the light chain variable region comprises a sequence having at least 90% sequence identity to the sequence as set forth in SEQ ID NO: 192;
wherein the heavy chain variable region comprises a sequence as set forth in SEQ ID NO: 193 and the light chain variable region comprises the sequence as set forth in SEQ ID NO: 194; or the heavy chain variable region comprises a sequence having at least 90% sequence identity to the sequence as set forth in SEQ ID NO: 193 and the light chain variable region comprises a sequence having at least 90% sequence identity to the sequence as set forth in SEQ ID NO: 194;
wherein the heavy chain variable region comprises a sequence as set forth in SEQ ID NO: 195 and the light chain variable region comprises the sequence as set forth in SEQ ID NO: 196; or the heavy chain variable region comprises a sequence having at least 90% sequence identity to the sequence as set forth in SEQ ID NO: 195 and the light chain variable region comprises a sequence having at least 90% sequence identity to the sequence as set forth in SEQ ID NO: 196;
wherein the heavy chain variable region comprises a sequence as set forth in SEQ ID NO: 197 and the light chain variable region comprises the sequence as set forth in SEQ ID NO: 198; or the heavy chain variable region comprises a sequence having at least 90% sequence identity to the sequence as set forth in SEQ ID NO: 197 and the light chain variable region comprises a sequence having at least 90% sequence identity to the sequence as set forth in SEQ ID NO: 198;
wherein the heavy chain variable region comprises a sequence as set forth in SEQ ID NO: 199 and the light chain variable region comprises the sequence as set forth in SEQ ID NO: 200; or the heavy chain variable region comprises a sequence having at least 90% sequence identity to the sequence as set forth in SEQ ID NO: 199 and the light chain variable region comprises a sequence having at least 90% sequence identity to the sequence as set forth in SEQ ID NO: 200;
wherein the heavy chain variable region comprises a sequence as set forth in SEQ ID NO: 1309 and the light chain variable region comprises the sequence as set forth in SEQ ID NO: 1310; or the heavy chain variable region comprises a sequence having at least 90% sequence identity to the sequence as set forth in SEQ ID NO: 1309 and the light chain variable region comprises a sequence having at least 90% sequence identity to the sequence as set forth in SEQ ID NO: 1310;
wherein the heavy chain variable region comprises a sequence as set forth in SEQ ID NO: 201 and the light chain variable region comprises the sequence as set forth in SEQ ID NO: 202; or the heavy chain variable region comprises a sequence having at least 90% sequence identity to the sequence as set forth in SEQ ID NO: 201 and the light chain variable region comprises a sequence having at least 90% sequence identity to the sequence as set forth in SEQ ID NO: 202;
wherein the heavy chain variable region comprises a sequence as set forth in SEQ ID NO: 203 and the light chain variable region comprises the sequence as set forth in SEQ ID NO: 204; or the heavy chain variable region comprises a sequence having at least 90% sequence identity to the sequence as set forth in SEQ ID NO: 203 and the light chain variable region comprises a sequence having at least 90% sequence identity to the sequence as set forth in SEQ ID NO: 204;
wherein the heavy chain variable region comprises a sequence as set forth in SEQ ID NO: 205 and the light chain variable region comprises the sequence as set forth in SEQ ID NO: 206; or the heavy chain variable region comprises a sequence having at least 90% sequence identity to the sequence as set forth in SEQ ID NO: 205 and the light chain variable region comprises a sequence having at least 90% sequence identity to the sequence as set forth in SEQ ID NO: 206;
wherein the heavy chain variable region comprises a sequence as set forth in SEQ ID NO: 1311 and the light chain variable region comprises the sequence as set forth in SEQ ID NO: 1312; or the heavy chain variable region comprises a sequence having at least 90% sequence identity to the sequence as set forth in SEQ ID NO: 1311 and the light chain variable region comprises a sequence having at least 90% sequence identity to the sequence as set forth in SEQ ID NO: 1312;
wherein the heavy chain variable region comprises a sequence as set forth in SEQ ID NO: 207 and the light chain variable region comprises the sequence as set forth in SEQ ID NO: 208; or the heavy chain variable region comprises a sequence having at least 90% sequence identity to the sequence as set forth in SEQ ID NO: 207 and the light chain variable region comprises a sequence having at least 90% sequence identity to the sequence as set forth in SEQ ID NO: 208;
wherein the heavy chain variable region comprises a sequence as set forth in SEQ ID NO: 209 and the light chain variable region comprises the sequence as set forth in SEQ ID NO: 210; or the heavy chain variable region comprises a sequence having at least 90% sequence identity to the sequence as set forth in SEQ ID NO: 209 and the light chain variable region comprises a sequence having at least 90% sequence identity to the sequence as set forth in SEQ ID NO: 210;
wherein the heavy chain variable region comprises a sequence as set forth in SEQ ID NO: 211 and the light chain variable region comprises the sequence as set forth in SEQ ID NO: 212; or the heavy chain variable region comprises a sequence having at least 90% sequence identity to the sequence as set forth in SEQ ID NO: 211 and the light chain variable region comprises a sequence having at least 90% sequence identity to the sequence as set forth in SEQ ID NO: 212;
wherein the heavy chain variable region comprises a sequence as set forth in SEQ ID NO: 213 and the light chain variable region comprises the sequence as set forth in SEQ ID NO: 214; or the heavy chain variable region comprises a sequence having at least 90% sequence identity to the sequence as set forth in SEQ ID NO: 213 and the light chain variable region comprises a sequence having at least 90% sequence identity to the sequence as set forth in SEQ ID NO: 214;
wherein the heavy chain variable region comprises a sequence as set forth in SEQ ID NO: 1313 and the light chain variable region comprises the sequence as set forth in SEQ ID NO: 1314; or the heavy chain variable region comprises a sequence having at least 90% sequence identity to the sequence as set forth in SEQ ID NO: 1313 and the light chain variable region comprises a sequence having at least 90% sequence identity to the sequence as set forth in SEQ ID NO: 1314;
wherein the heavy chain variable region comprises a sequence as set forth in SEQ ID NO: 215 and the light chain variable region comprises the sequence as set forth in SEQ ID NO: 216; or the heavy chain variable region comprises a sequence having at least 90% sequence identity to the sequence as set forth in SEQ ID NO: 215 and the light chain variable region comprises a sequence having at least 90% sequence identity to the sequence as set forth in SEQ ID NO: 216;
wherein the heavy chain variable region comprises a sequence as set forth in SEQ ID NO: 217 and the light chain variable region comprises the sequence as set forth in SEQ ID NO: 218; or the heavy chain variable region comprises a sequence having at least 90% sequence identity to the sequence as set forth in SEQ ID NO: 217 and the light chain variable region comprises a sequence having at least 90% sequence identity to the sequence as set forth in SEQ ID NO: 218;
wherein the heavy chain variable region comprises a sequence as set forth in SEQ ID NO: 219 and the light chain variable region comprises the sequence as set forth in SEQ ID NO: 220; or the heavy chain variable region comprises a sequence having at least 90% sequence identity to the sequence as set forth in SEQ ID NO: 219 and the light chain variable region comprises a sequence having at least 90% sequence identity to the sequence as set forth in SEQ ID NO: 220;
wherein the heavy chain variable region comprises a sequence as set forth in SEQ ID NO: 221 and the light chain variable region comprises the sequence as set forth in SEQ ID NO: 222; or the heavy chain variable region comprises a sequence having at least 90% sequence identity to the sequence as set forth in SEQ ID NO: 221 and the light chain variable region comprises a sequence having at least 90% sequence identity to the sequence as set forth in SEQ ID NO: 222;
wherein the heavy chain variable region comprises a sequence as set forth in SEQ ID NO: 223 and the light chain variable region comprises the sequence as set forth in SEQ ID NO: 224; or the heavy chain variable region comprises a sequence having at least 90% sequence identity to the sequence as set forth in SEQ ID NO: 223 and the light chain variable region comprises a sequence having at least 90% sequence identity to the sequence as set forth in SEQ ID NO: 224;
wherein the heavy chain variable region comprises a sequence as set forth in SEQ ID NO: 225 and the light chain variable region comprises the sequence as set forth in SEQ ID NO: 226; or the heavy chain variable region comprises a sequence having at least 90% sequence identity to the sequence as set forth in SEQ ID NO: 225 and the light chain variable region comprises a sequence having at least 90% sequence identity to the sequence as set forth in SEQ ID NO: 226;
wherein the heavy chain variable region comprises a sequence as set forth in SEQ ID NO: 27 and the light chain variable region comprises the sequence as set forth in SEQ ID NO: 228; or the heavy chain variable region comprises a sequence having at least 90% sequence identity to the sequence as set forth in SEQ ID NO: 227 and the light chain variable region comprises a sequence having at least 90% sequence identity to the sequence as set forth in SEQ ID NO: 228;
wherein the heavy chain variable region comprises a sequence as set forth in SEQ ID NO: 229 and the light chain variable region comprises the sequence as set forth in SEQ ID NO: 230; or the heavy chain variable region comprises a sequence having at least 90% sequence identity to the sequence as set forth in SEQ ID NO: 229 and the light chain variable region comprises a sequence having at least 90% sequence identity to the sequence as set forth in SEQ ID NO: 230;
wherein the heavy chain variable region comprises a sequence as set forth in SEQ ID NO: 231 and the light chain variable region comprises the sequence as set forth in SEQ ID NO: 232; or the heavy chain variable region comprises a sequence having at least 90% sequence identity to the sequence as set forth in SEQ ID NO: 231 and the light chain variable region comprises a sequence having at least 90% sequence identity to the sequence as set forth in SEQ ID NO: 232;
wherein the heavy chain variable region comprises a sequence as set forth in SEQ ID NO: 233 and the light chain variable region comprises the sequence as set forth in SEQ ID NO: 234; or the heavy chain variable region comprises a sequence having at least 90% sequence identity to the sequence as set forth in SEQ ID NO: 233 and the light chain variable region comprises a sequence having at least 90% sequence identity to the sequence as set forth in SEQ ID NO: 234;
wherein the heavy chain variable region comprises a sequence as set forth in SEQ ID NO: 235 and the light chain variable region comprises the sequence as set forth in SEQ ID NO: 236; or the heavy chain variable region comprises a sequence having at least 90% sequence identity to the sequence as set forth in SEQ ID NO: 235 and the light chain variable region comprises a sequence having at least 90% sequence identity to the sequence as set forth in SEQ ID NO: 236;
wherein the heavy chain variable region comprises a sequence as set forth in SEQ ID NO: 237 and the light chain variable region comprises the sequence as set forth in SEQ ID NO: 238; or the heavy chain variable region comprises a sequence having at least 90% sequence identity to the sequence as set forth in SEQ ID NO: 237 and the light chain variable region comprises a sequence having at least 90% sequence identity to the sequence as set forth in SEQ ID NO: 238;
wherein the heavy chain variable region comprises a sequence as set forth in SEQ ID NO: 239 and the light chain variable region comprises the sequence as set forth in SEQ ID NO: 240; or the heavy chain variable region comprises a sequence having at least 90% sequence identity to the sequence as set forth in SEQ ID NO: 239 and the light chain variable region comprises a sequence having at least 90% sequence identity to the sequence as set forth in SEQ ID NO: 240;
wherein the heavy chain variable region comprises a sequence as set forth in SEQ ID NO: 241 and the light chain variable region comprises the sequence as set forth in SEQ ID NO: 242; or the heavy chain variable region comprises a sequence having at least 90% sequence identity to the sequence as set forth in SEQ ID NO: 241 and the light chain variable region comprises a sequence having at least 90% sequence identity to the sequence as set forth in SEQ ID NO: 242;
wherein the heavy chain variable region comprises a sequence as set forth in SEQ ID NO: 243 and the light chain variable region comprises the sequence as set forth in SEQ ID NO: 244; or the heavy chain variable region comprises a sequence having at least 90% sequence identity to the sequence as set forth in SEQ ID NO: 243 and the light chain variable region comprises a sequence having at least 90% sequence identity to the sequence as set forth in SEQ ID NO: 244;
wherein the heavy chain variable region comprises a sequence as set forth in SEQ ID NO: 245 and the light chain variable region comprises the sequence as set forth in SEQ ID NO: 246; or the heavy chain variable region comprises a sequence having at least 90% sequence identity to the sequence as set forth in SEQ ID NO: 245 and the light chain variable region comprises a sequence having at least 90% sequence identity to the sequence as set forth in SEQ ID NO: 246; or
wherein the heavy chain variable region comprises a sequence as set forth in SEQ ID NO: 247 and the light chain variable region comprises the sequence as set forth in SEQ ID NO: 248; or the heavy chain variable region comprises a sequence having at least 90% sequence identity to the sequence as set forth in SEQ ID NO: 247 and the light chain variable region comprises a sequence having at least 90% sequence identity to the sequence as set forth in SEQ ID NO: 248.

206. The antibody or fragment thereof of claim 203, wherein the antibody or fragment thereof further comprises a human heavy chain constant region and/or a human light chain constant.

207. The antibody or fragment thereof of claim 206, wherein the human heavy chain constant region comprises a human IgG1 heavy chain constant region, a human IgG4 heavy chain constant region, a human IgG2 heavy chain constant region, or a human IgG3 heavy chain constant region; and
wherein the human light chain constant region comprises a human Igκ light chain constant region or a human Igλ light chain constant region.

208. An engineered T cell receptor (eTCR) comprising a constant alpha domain (Cα), a constant beta domain (Cβ), and the antibody or fragment thereof of claim 203 linked to the Ca domain or the Cβ domain.

209. The eTCR of claim 208, wherein the antibody or fragment thereof of claim 203 is an scFv.

210. The eTCR of claim 208, wherein one antibody or fragment thereof of claim 203 is linked to the Cα domain and one antibody or fragment thereof of claim 203 is linked to the Cβ domain.

211. A detection assay comprising an antibody or fragment thereof of claim 203 linked to a solid support via a covalent interaction, a noncovalent interaction, or passive binding.

212. The detection assay of claim 211, wherein the solid support comprises slides, wells, chips, pins, filters, beads, or membrane.

213. The detection assay of claim 211, wherein the solid substrate comprises polystyrene, glass, gold, nylon, nitrocellulose, polyvinylidene fluoride (PVDF), paper, or latex.

214. A nucleic acid encoding an antibody or fragment thereof of claim 203.

215. A method of treating a subject in need thereof comprising administering a therapeutically effective amount of the antibody or fragment thereof of claim 203 to the subject, thereby treating the subject in need thereof.

216. The method of claim 215, wherein the therapeutically effective amount provides a prophylactic or a therapeutic treatment against a Merkel cell carcinoma.

217. A method of detecting a T antigen of the Merkel cell polyomavirus (T antigen) comprising administering to a subject or a biological sample derived from the subject a detection molecule comprising the antibody or fragment thereof of claim 203 and detecting the detection molecule.

218. The method of claim 217, wherein the detecting comprises imaging.

219. The method of claim 217, further comprising diagnosing the subject based on the detecting by determining the level of expression of the T antigen based on a signal from the detectable molecule and comparing the level of expression of the detectable molecule to a reference level.

220. The method of claim 219, wherein the reference level is
the level of the signal from a tissue without a T antigen-expressing cell or
the level of the signal from the subject or the biological sample derived from the subject at a different time point.

221. The method of claim 217, wherein the biological sample derived from the subject comprises a tissue biopsy, a tumor tissue biopsy, or a skin biopsy.

222. A method, comprising:
obtaining a sample derived from a tumor-infiltrated lymph node derived from a subject;
assessing the sample for the presence of T-Ag specific germinal center B cells; and
administering an MCC treatment to the subject if T-Ag specific germinal center B cells are not present in the sample.
